SEQUENCE LISTING (179 sequences)

SEQ ID NO: 1 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
cgugugcacuucgcuucaccu

SEQ ID NO: 2 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
aggugaagcgaagugcacacgguuu

SEQ ID NO: 3 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
ugcacuucgcuucaccu

SEQ ID NO: 4 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
aggugaagcgaagugcacacgu

SEQ ID NO: 5 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
ugcacuucgcuucaccu

SEQ ID NO: 6 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
aggugaagcgaagugcacacgu

SEQ ID NO: 7 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-Fluoro nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
ugcacuucgcuucaccu

SEQ ID NO: 8 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
aggugaagcgaagugcacacgu

SEQ ID NO: 9 - RNA, 21 nt, Artificial Sequence
  Description of Artificial Sequence Synthetic oligonucleotide
  modified_base (1)..(1): 2′-Fluoro nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucacc

SEQ ID NO: 10 - RNA, 23 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-Fluoro nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ggugaagcgaagugcacacgguc

SEQ ID NO: 11 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucacc

SEQ ID NO: 12 - RNA, 23 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ggugaagcgaagugcacacgguc

SEQ ID NO: 13 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-O-Methyl nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucacc

SEQ ID NO: 14 - RNA, 23 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ggugaagcgaagugcacacgguc

SEQ ID NO: 15 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucacc

SEQ ID NO: 16 - RNA, 23 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-Fluoro nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ggugaagcgaagugcacacgguc

SEQ ID NO: 17 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucacc

SEQ ID NO: 18 - RNA, 23 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ggugaagcgaagugcacacgguc

SEQ ID NO: 19 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucacc

SEQ ID NO: 20 - RNA, 23 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ggugaagcgaagugcacacgguc

SEQ ID NO: 21 - RNA, 21 nt, Artificial Sequence
  Description of Artificial Sequence Synthetic oligonucleotide
  modified_base (1)..(1): 2′-Fluoro nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucacc

SEQ ID NO: 22 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-Fluoro nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ggugaagcgaagugcacacggucuu

SEQ ID NO: 23 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucacc

SEQ ID NO: 24 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ggugaagcgaagugcacacggucuu

SEQ ID NO: 25 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-O-Methyl nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucacc

SEQ ID NO: 26 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ggugaagcgaagugcacacggucuu

SEQ ID NO: 27 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucacc

SEQ ID NO: 28 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-Fluoro nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ggugaagcgaagugcacacggucuu

SEQ ID NO: 29 - RNA, 17 nt, Artificial Sequence
  Description of Artificial Sequence Synthetic oligonucleotide
  modified_base (1)..(1): 2′-Fluoro nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
gugcacuucgcuucacc

SEQ ID NO: 30 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  misc_feature (19)..(20): Phosphorothioate linker
  modified_base (20)..(20): 2′-Fluoro nucleotide
  misc_feature (20)..(21): Phosphorothioate linker
  modified_base (21)..(21): 2′-O-Methyl nucleotide
ggugaagcgaagugcacacggu

SEQ ID NO: 31 - RNA, 17 nt, Artificial Sequence
  Description of Artificial Sequence Synthetic oligonucleotide
  modified_base (1)..(1): 2′-Fluoro nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
gugcacuucgcuucacc

SEQ ID NO: 32 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  misc_feature (19)..(20): Phosphorothioate linker
  modified_base (20)..(20): 2′-Fluoro nucleotide
  misc_feature (20)..(21): Phosphorothioate linker
  modified_base (21)..(21): 2′-O-Methyl nucleotide
ggugaagcgaagugcacacgg

SEQ ID NO: 33 - RNA, 17 nt, Artificial Sequence
  Description of Artificial Sequence Synthetic oligonucleotide
  modified_base (1)..(1): 2′-Fluoro nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
gugcacuucgcuucacc

SEQ ID NO: 34 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  misc_feature (17)..(18): Phosphorothioate linker
  modified_base (18)..(18): 2′-Fluoro nucleotide
  misc_feature (18)..(19): Phosphorothioate linker
  modified_base (19)..(19): 2′-O-Methyl nucleotide
  misc_feature (19)..(20): Phosphorothioate linker
  modified_base (20)..(20): 2′-Fluoro nucleotide
  misc_feature (20)..(21): Phosphorothioate linker
  modified_base (21)..(21): 2′-O-Methyl nucleotide
ggugaagcgaagugcacacgg

SEQ ID NO: 35 - RNA, 21 nt, Artificial Sequence
  Description of Artificial Sequence Synthetic oligonucleotide
  modified_base (1)..(1): 2′-Fluoro nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucaca

SEQ ID NO: 36 - RNA, 23 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-Fluoro nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ugugaagcgaagugcacacgguc

SEQ ID NO: 37 - RNA, 21 nt, Artificial Sequence
  Description of Artificial Sequence Synthetic oligonucleotide
  modified_base (1)..(1): 2′-Fluoro nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucaca

SEQ ID NO: 38 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-Fluoro nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ugugaagcgaagugcacacggucuu

SEQ ID NO: 39 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucaca

SEQ ID NO: 40 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ugugaagcgaagugcacacggucuu

SEQ ID NO: 41 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-O-Methyl nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucaca

SEQ ID NO: 42 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ugugaagcgaagugcacacggucuu

SEQ ID NO: 43 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucaca

SEQ ID NO: 44 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-Fluoro nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ugugaagcgaagugcacacggucuu

SEQ ID NO: 45 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ccgugugcacuucgcuucaca

SEQ ID NO: 46 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ugugaagcgaagugcacacggucuu

SEQ ID NO: 47 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
gugcacuucgcuucaca

SEQ ID NO: 48 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
ugugaagcgaagugcacacggu

SEQ ID NO: 49 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
gugcacuucgcuucaca

SEQ ID NO: 50 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
ugugaagcgaagugcacacggu

SEQ ID NO: 51 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
gugcacuucgcuucaca

SEQ ID NO: 52 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
ugugaagcgaagugcacacggu

SEQ ID NO: 53 - RNA, 17 nt, Artificial Sequence
  Description of Artificial Sequence Synthetic oligonucleotide
  modified_base (1)..(1): 2′-Fluoro nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
gugcacuucgcuucaca

SEQ ID NO: 54 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  misc_feature (19)..(20): Phosphorothioate linker
  modified_base (20)..(20): 2′-Fluoro nucleotide
  misc_feature (20)..(21): Phosphorothioate linker
  modified_base (21)..(21): 2′-O-Methyl nucleotide
ugugaagcgaagugcacacgg

SEQ ID NO: 55 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ucgcuucaccucugcacgucg

SEQ ID NO: 56 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
cgacgugcagaggugaagcgaaguu

SEQ ID NO: 57 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ucgcuucaccucugcacguca

SEQ ID NO: 58 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ugacgugcagaggugaagcgaaguu

SEQ ID NO: 59 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-O-Methyl nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
ucgcuucaccucugcacguca

SEQ ID NO: 60 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ugacgugcagaggugaagcgaaguu

SEQ ID NO: 61 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
uucaccucugcacguca

SEQ ID NO: 62 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
ugacgugcagaggugaagcgau

SEQ ID NO: 63 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
uucaccucugcacguca

SEQ ID NO: 64 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
ugacgugcagaggugaagcgau

SEQ ID NO: 65 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
uucaccucugcacguca

SEQ ID NO: 66 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
ugacgugcagaggugaagcgau

SEQ ID NO: 67 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
uuuacuagugccauuuguuca

SEQ ID NO: 68 - RNA, 23 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-Fluoro nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ugaacaaauggcacuaguaaacu

SEQ ID NO: 69 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
uuuacuagugccauuuguuca

SEQ ID NO: 70 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-Fluoro nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ugaacaaauggcacuaguaaacuuu

SEQ ID NO: 71 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-Fluoro nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
uuuacuagugccauuuguuca

SEQ ID NO: 72 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ugaacaaauggcacuaguaaacuuu

SEQ ID NO: 73 - RNA, 21 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-O-Methyl nucleotide
  modified_base (11)..(11): 2′-Fluoro nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
uuuacuagugccauuuguuca

SEQ ID NO: 74 - RNA, 25 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-Fluoro nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (20)..(20): 2′-O-Methyl nucleotide
  modified_base (21)..(21): 2′-O-Methyl nucleotide
  misc_feature (21)..(22): Phosphorothioate linker
  modified_base (22)..(22): 2′-O-Methyl nucleotide
  misc_feature (22)..(23): Phosphorothioate linker
  modified_base (23)..(23): 2′-O-Methyl nucleotide
ugaacaaauggcacuaguaaacuuu

SEQ ID NO: 75 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
ugcacuucgcuucaccu

SEQ ID NO: 76 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
gugcacuucgcuucaca

SEQ ID NO: 77 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
aggugaagcgaagugcacacgu

SEQ ID NO: 78 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
ugugaagcgaagugcacacggu

SEQ ID NO: 79 - RNA, 21 nt, Artificial Sequence
  Description of Artificial Sequence Synthetic oligonucleotide
  modified_base (1)..(1): Unlocked nucleobase analogue
  modified_base (2)..(2): 2′-O-Methyl nucleotide
  modified_base (4)..(4): 2′-O-Methyl nucleotide
  modified_base (7)..(7): 2′-O-Methyl nucleotide
  modified_base (8)..(8): 2′-O-Methyl nucleotide
  modified_base (14)..(14): 2′-O-Methyl nucleotide
  modified_base (16)..(16): 2′-O-Methyl nucleotide
  modified_base (20)..(20): Unlocked nucleobase analogue
  modified_base (21)..(21): Unlocked nucleobase analogue
agguauguugcccguuuguuu

SEQ ID NO: 80 - RNA, 21 nt, Artificial Sequence
  Description of Artificial Sequence Synthetic oligonucleotide
  modified_base (7)..(7): 2′-O-Methyl nucleotide
  modified_base (15)..(15): 2′-O-Methyl nucleotide
  modified_base (20)..(20): Unlocked nucleobase analogue
  modified_base (21)..(21): Unlocked nucleobase analogue
acaaacgggcaacauaccuuu

SEQ ID NO: 81 - RNA, 21 nt, Artificial Sequence
  Description of Artificial Sequence Synthetic oligonucleotide
  modified_base (1)..(1): Unlocked nucleobase analogue
  modified_base (3)..(3): 2′-O-Methyl nucleotide
  modified_base (4)..(4): 2′-O-Methyl nucleotide
  modified_base (6)..(6): 2′-O-Methyl nucleotide
  modified_base (18)..(18): 2′-O-Methyl nucleotide
  modified_base (21)..(21): Unlocked nucleobase analogue
gcucaguuuacuagugccauu

SEQ ID NO: 82 - RNA, 21 nt, Artificial Sequence
  Description of Artificial Sequence Synthetic oligonucleotide
  modified_base (10)..(10): 2′-O-Methyl nucleotide
  modified_base (18)..(18): 2′-O-Methyl nucleotide
uggcacuaguaaacugagcuu

SEQ ID NO: 83 - RNA, 21 nt, Artificial Sequence
  Description of Artificial Sequence Synthetic oligonucleotide
  modified_base (1)..(1): Unlocked nucleobase analogue
  modified_base (5)..(5): 2′-O-Methyl nucleotide
  modified_base (6)..(6): 2′-O-Methyl nucleotide
  modified_base (12)..(12): 2′-O-Methyl nucleotide
  modified_base (16)..(16): 2′-O-Methyl nucleotide
  modified_base (17)..(17): 2′-O-Methyl nucleotide
  modified_base (20)..(20): Unlocked nucleobase analogue
  modified_base (21)..(21): Unlocked nucleobase analogue
ccgugugcacuucgcuucauu

SEQ ID NO: 84 - RNA, 21 nt, Artificial Sequence
  Description of Artificial Sequence Synthetic oligonucleotide
  modified_base (12)..(12): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
ugaagcgaagugcacacgguu

SEQ ID NO: 85 - RNA, 21 nt, Artificial Sequence
  Description of Artificial Sequence Synthetic oligonucleotide
  modified_base (1)..(1): Unlocked nucleobase analogue
  modified_base (3)..(3): 2′-O-Methyl nucleotide
  modified_base (4)..(4): 2′-O-Methyl nucleotide
  modified_base (6)..(6): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
gcucaguuuacuagugccauu

SEQ ID NO: 86 - RNA, 21 nt, Artificial Sequence
  Description of Artificial Sequence Synthetic oligonucleotide
  modified_base (10)..(10): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
uggcacuaguaaacugagcuu

SEQ ID NO: 87 - RNA, 21 nt, Artificial Sequence
  Description of Artificial Sequence Synthetic oligonucleotide
  modified_base (1)..(1): Unlocked nucleobase analogue
  modified_base (5)..(5): 2′-O-Methyl nucleotide
  modified_base (6)..(6): 2′-O-Methyl nucleotide
  modified_base (12)..(12): 2′-O-Methyl nucleotide
  modified_base (16)..(16): 2′-O-Methyl nucleotide
  modified_base (21)..(21): Unlocked nucleobase analogue
ccgugugcacuucgcuucauu

SEQ ID NO: 88 - RNA, 21 nt, Artificial Sequence
  Description of Artificial Sequence Synthetic oligonucleotide
  modified_base (12)..(12): 2′-O-Methyl nucleotide
  modified_base (18)..(18): 2′-O-Methyl nucleotide
ugaagcgaagugcacacgguu

SEQ ID NO: 89 - RNA, 21 nt, Artificial Sequence
  Description of Artificial Sequence Synthetic oligonucleotide
  modified_base (1)..(1): Unlocked nucleobase analogue
  modified_base (2)..(2): 2′-O-Methyl nucleotide
  modified_base (3)..(3): 2′-O-Methyl nucleotide
  modified_base (4)..(4): 2′-O-Methyl nucleotide
  modified_base (15)..(15): 2′-O-Methyl nucleotide
  modified_base (17)..(17): 2′-O-Methyl nucleotide
  modified_base (21)..(21): Unlocked nucleobase analogue
cuggcucaguuuacuaguguu

SEQ ID NO: 90 - RNA, 21 nt, Artificial Sequence
  Description of Artificial Sequence Synthetic oligonucleotide
  modified_base (6)..(6): 2′-O-Methyl nucleotide
  modified_base (13)..(13): 2′-O-Methyl nucleotide
  modified_base (15)..(15): 2′-O-Methyl nucleotide
cacuaguaaacugagccaguu

SEQ ID NO: 91 - RNA, 21 nt, Artificial Sequence
  Description of Artificial Sequence Synthetic oligonucleotide
  modified_base (1)..(1): Unlocked nucleobase analogue
  modified_base (5)..(5): 2′-O-Methyl nucleotide
  modified_base (6)..(6): 2′-O-Methyl nucleotide
  modified_base (12)..(12): 2′-O-Methyl nucleotide
  modified_base (16)..(16): 2′-O-Methyl nucleotide
  modified_base (20)..(20): Unlocked nucleobase analogue
  modified_base (21)..(21): Unlocked nucleobase analogue
ccgugugcacuucgcuucauu

SEQ ID NO: 92 - RNA, 21 nt, Artificial Sequence
  Description of Artificial Sequence Synthetic oligonucleotide
  modified_base (12)..(12): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
ugaagcgaagugcacacgguu

SEQ ID NO: 93 - RNA, 21 nt, Artificial Sequence
  Description of Artificial Sequence Synthetic oligonucleotide
  modified_base (1)..(1): Unlocked nucleobase analogue
  modified_base (3)..(3): 2′-O-Methyl nucleotide
  modified_base (6)..(6): 2′-O-Methyl nucleotide
  modified_base (12)..(12): 2′-O-Methyl nucleotide
  modified_base (14)..(14): 2′-O-Methyl nucleotide
  modified_base (20)..(20): Unlocked nucleobase analogue
  modified_base (21)..(21): Unlocked nucleobase analogue
gcucaguuuacuagugccauu

SEQ ID NO: 94 - RNA, 21 nt, Artificial Sequence
  Description of Artificial Sequence Synthetic oligonucleotide
  modified_base (15)..(15): 2′-O-Methyl nucleotide
  modified_base (20)..(20): Unlocked nucleobase analogue
  modified_base (21)..(21): Unlocked nucleobase analogue
uggcacuaguaaacugagcuu

SEQ ID NO: 95 - RNA, 21 nt, Artificial Sequence
  Description of Artificial Sequence Synthetic oligonucleotide
  modified_base (1)..(1): Unlocked nucleobase analogue
  modified_base (2)..(2): 2′-O-Methyl nucleotide
  modified_base (4)..(4): 2′-O-Methyl nucleotide
  modified_base (8)..(8): 2′-O-Methyl nucleotide
  modified_base (14)..(14): 2′-O-Methyl nucleotide
  modified_base (16)..(16): 2′-O-Methyl nucleotide
  modified_base (20)..(20): Unlocked nucleobase analogue
  modified_base (21)..(21): Unlocked nucleobase analogue
agguauguugcccguuuguuu

SEQ ID NO: 96 - RNA, 21 nt, Artificial Sequence
  Description of Artificial Sequence Synthetic oligonucleotide
  modified_base (7)..(7): 2′-O-Methyl nucleotide
  modified_base (15)..(15): 2′-O-Methyl nucleotide
  modified_base (19)..(19): 2′-O-Methyl nucleotide
  modified_base (20)..(20): Unlocked nucleobase analogue
  modified_base (21)..(21): Unlocked nucleobase analogue
acaaacgggcaacauaccuuu

SEQ ID NO: 97 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-Methyl nucleotide
  modified_base (6)..(6): 2′-O-Methyl nucleotide
  modified_base (13)..(13): 2′-O-Methyl nucleotide
  modified_base (14)..(14): 2′-O-Methyl nucleotide
  modified_base (16)..(16): 2′-O-Methyl nucleotide
  modified_base (17)..(17): 2′-O-Methyl nucleotide
  modified_base (20)..(20): Unlocked nucleobase analogue
  modified_base (21)..(21): Unlocked nucleobase analogue
gccgauccauacugcggaauu

SEQ ID NO: 98 - RNA, 21 nt, Artificial Sequence
  Description of Artificial Sequence Synthetic oligonucleotide
  modified_base (8)..(8): 2′-O-Methyl nucleotide
  modified_base (13)..(13): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
uuccgcaguauggaucggcuu

SEQ ID NO: 99 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-Methyl nucleotide
  modified_base (6)..(6): 2′-O-Methyl nucleotide
  modified_base (13)..(13): 2′-O-Methyl nucleotide
  modified_base (14)..(14): 2′-O-Methyl nucleotide
  modified_base (16)..(16): 2′-O-Methyl nucleotide
  modified_base (17)..(17): 2′-O-Methyl nucleotide
  modified_base (21)..(21): Unlocked nucleobase analogue
gccgauccauacugcggaauu

SEQ ID NO: 100 - RNA, 21 nt, Artificial Sequence
  Description of Artificial Sequence Synthetic oligonucleotide
  modified_base (8)..(8): 2′-O-Methyl nucleotide
  modified_base (13)..(13): 2′-O-Methyl nucleotide
  modified_base (18)..(18): 2′-O-Methyl nucleotide
uuccgcaguauggaucggcuu

SEQ ID NO: 101 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-Methyl nucleotide
  modified_base (6)..(6): 2′-O-Methyl nucleotide
  modified_base (13)..(13): 2′-O-Methyl nucleotide
  modified_base (14)..(14): 2′-O-Methyl nucleotide
  modified_base (17)..(17): 2′-O-Methyl nucleotide
  modified_base (21)..(21): Unlocked nucleobase analogue
gccgauccauacugcggaauu

SEQ ID NO: 102 - RNA, 21 nt, Artificial Sequence
  Description of Artificial Sequence Synthetic oligonucleotide
  modified_base (8)..(8): 2′-O-Methyl nucleotide
  modified_base (13)..(13): 2′-O-Methyl nucleotide
  modified_base (18)..(18): 2′-O-Methyl nucleotide
uuccgcaguauggaucggcuu

SEQ ID NO: 103 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-Methyl nucleotide
  modified_base (6)..(6): 2′-O-Methyl nucleotide
  modified_base (13)..(13): 2′-O-Methyl nucleotide
  modified_base (14)..(14): 2′-O-Methyl nucleotide
  modified_base (17)..(17): 2′-O-Methyl nucleotide
  modified_base (20)..(20): Unlocked nucleobase analogue
  modified_base (21)..(21): Unlocked nucleobase analogue
gccgauccauacugcggaauu

SEQ ID NO: 104 - RNA, 21 nt, Artificial Sequence
  Description of Artificial Sequence Synthetic oligonucleotide
  modified_base (8)..(8): 2′-O-Methyl nucleotide
  modified_base (13)..(13): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
uuccgcaguauggaucggcuu

SEQ ID NO: 105 - RNA, 21 nt, Artificial Sequence
  Description of Artificial Sequence Synthetic oligonucleotide
  modified_base (1)..(1): Unlocked nucleobase analogue
  modified_base (3)..(3): 2′-O-Methyl nucleotide
  modified_base (6)..(6): 2′-O-Methyl nucleotide
  modified_base (12)..(12): 2′-O-Methyl nucleotide
  modified_base (14)..(14): 2′-O-Methyl nucleotide
  modified_base (21)..(21): Unlocked nucleobase analogue
gcucaguuuacuagugccauu

SEQ ID NO: 106 - RNA, 21 nt, Artificial Sequence
  Description of Artificial Sequence Synthetic oligonucleotide
  modified_base (15)..(15): 2′-O-Methyl nucleotide
uggcacuaguaaacugagcuu

SEQ ID NO: 107 - RNA, 21 nt, Artificial Sequence
  Description of Artificial Sequence Synthetic oligonucleotide
  modified_base (1)..(1): Unlocked nucleobase analogue
  modified_base (2)..(2): 2′-O-Methyl nucleotide
  modified_base (3)..(3): 2′-O-Methyl nucleotide
  modified_base (6)..(6): 2′-O-Methyl nucleotide
  modified_base (12)..(12): 2′-O-Methyl nucleotide
  modified_base (20)..(20): Unlocked nucleobase analogue
  modified_base (21)..(21): Unlocked nucleobase analogue
cuggcucaguuuacuaguguu

SEQ ID NO: 108 - RNA, 21 nt, Artificial Sequence
  Description of Artificial Sequence Synthetic oligonucleotide
  modified_base (15)..(15): 2′-O-Methyl nucleotide
  modified_base (20)..(20): Unlocked nucleobase analogue
  modified_base (21)..(21): Unlocked nucleobase analogue
cacuaguaaacugagccaguu

SEQ ID NO: 109 - RNA, 21 nt, Artificial Sequence
  Description of Artificial Sequence Synthetic oligonucleotide
  modified_base (1)..(1): Unlocked nucleobase analogue
  modified_base (2)..(2): 2′-O-Methyl nucleotide
  modified_base (4)..(4): 2′-O-Methyl nucleotide
  modified_base (7)..(7): 2′-O-Methyl nucleotide
  modified_base (8)..(8): 2′-O-Methyl nucleotide
  modified_base (14)..(14): 2′-O-Methyl nucleotide
  modified_base (16)..(16): 2′-O-Methyl nucleotide
  modified_base (20)..(20): Unlocked nucleobase analogue
  modified_base (21)..(21): Unlocked nucleobase analogue
agguauguugcccguuuguuu

SEQ ID NO: 110 - RNA, 21 nt, Artificial Sequence
  Description of Artificial Sequence Synthetic oligonucleotide
  modified_base (1)..(1): Unlocked nucleobase analogue
  modified_base (3)..(3): 2′-O-Methyl nucleotide
  modified_base (4)..(4): 2′-O-Methyl nucleotide
  modified_base (6)..(6): 2′-O-Methyl nucleotide
  modified_base (18)..(18): 2′-O-Methyl nucleotide
  modified_base (21)..(21): Unlocked nucleobase analogue
gcucaguuuacuagugccauu

SEQ ID NO: 111 - RNA, 21 nt, Artificial Sequence
  Description of Artificial Sequence Synthetic oligonucleotide
  modified_base (1)..(1): Unlocked nucleobase analogue
  modified_base (5)..(5): 2′-O-Methyl nucleotide
  modified_base (6)..(6): 2′-O-Methyl nucleotide
  modified_base (12)..(12): 2′-O-Methyl nucleotide
  modified_base (16)..(16): 2′-O-Methyl nucleotide
  modified_base (17)..(17): 2′-O-Methyl nucleotide
  modified_base (20)..(20): Unlocked nucleobase analogue
  modified_base (21)..(21): Unlocked nucleobase analogue
ccgugugcacuucgcuucauu

SEQ ID NO: 112 - RNA, 21 nt, Artificial Sequence
  Description of Artificial Sequence Synthetic oligonucleotide
  modified_base (1)..(1): Unlocked nucleobase analogue
  modified_base (3)..(3): 2′-O-Methyl nucleotide
  modified_base (4)..(4): 2′-O-Methyl nucleotide
  modified_base (6)..(6): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
gcucaguuuacuagugccauu

SEQ ID NO: 113 - RNA, 21 nt, Artificial Sequence
  Description of Artificial Sequence Synthetic oligonucleotide
  modified_base (1)..(1): Unlocked nucleobase analogue
  modified_base (5)..(5): 2′-O-Methyl nucleotide
  modified_base (6)..(6): 2′-O-Methyl nucleotide
  modified_base (12)..(12): 2′-O-Methyl nucleotide
  modified_base (16)..(16): 2′-O-Methyl nucleotide
  modified_base (21)..(21): Unlocked nucleobase analogue
ccgugugcacuucgcuucauu

SEQ ID NO: 114 - RNA, 21 nt, Artificial Sequence
  Description of Artificial Sequence Synthetic oligonucleotide
  modified_base (1)..(1): Unlocked nucleobase analogue
  modified_base (2)..(2): 2′-O-Methyl nucleotide
  modified_base (3)..(3): 2′-O-Methyl nucleotide
  modified_base (4)..(4): 2′-O-Methyl nucleotide
  modified_base (15)..(15): 2′-O-Methyl nucleotide
  modified_base (17)..(17): 2′-O-Methyl nucleotide
  modified_base (21)..(21): Unlocked nucleobase analogue
cuggcucaguuuacuaguguu

SEQ ID NO: 115 - RNA, 21 nt, Artificial Sequence
  Description of Artificial Sequence Synthetic oligonucleotide
  modified_base (1)..(1): Unlocked nucleobase analogue
  modified_base (5)..(5): 2′-O-Methyl nucleotide
  modified_base (6)..(6): 2′-O-Methyl nucleotide
  modified_base (12)..(12): 2′-O-Methyl nucleotide
  modified_base (16)..(16): 2′-O-Methyl nucleotide
  modified_base (20)..(20): Unlocked nucleobase analogue
  modified_base (21)..(21): Unlocked nucleobase analogue
ccgugugcacuucgcuucauu

SEQ ID NO: 116 - RNA, 21 nt, Artificial Sequence
  Description of Artificial Sequence Synthetic oligonucleotide
  modified_base (1)..(1): Unlocked nucleobase analogue
  modified_base (3)..(3): 2′-O-Methyl nucleotide
  modified_base (6)..(6): 2′-O-Methyl nucleotide
  modified_base (12)..(12): 2′-O-Methyl nucleotide
  modified_base (14)..(14): 2′-O-Methyl nucleotide
  modified_base (20)..(20): Unlocked nucleobase analogue
  modified_base (21)..(21): Unlocked nucleobase analogue
gcucaguuuacuagugccauu

SEQ ID NO: 117 - RNA, 21 nt, Artificial Sequence
  Description of Artificial Sequence Synthetic oligonucleotide
  modified_base (1)..(1): Unlocked nucleobase analogue
  modified_base (2)..(2): 2′-O-Methyl nucleotide
  modified_base (4)..(4): 2′-O-Methyl nucleotide
  modified_base (8)..(8): 2′-O-Methyl nucleotide
  modified_base (14)..(14): 2′-O-Methyl nucleotide
  modified_base (16)..(16): 2′-O-Methyl nucleotide
  modified_base (20)..(20): Unlocked nucleobase analogue
  modified_base (21)..(21): Unlocked nucleobase analogue
agguauguugcccguuuguuu

SEQ ID NO: 118 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-Methyl nucleotide
  modified_base (6)..(6): 2′-O-Methyl nucleotide
  modified_base (13)..(13): 2′-O-Methyl nucleotide
  modified_base (14)..(14): 2′-O-Methyl nucleotide
  modified_base (16)..(16): 2′-O-Methyl nucleotide
  modified_base (17)..(17): 2′-O-Methyl nucleotide
  modified_base (20)..(20): Unlocked nucleobase analogue
  modified_base (21)..(21): Unlocked nucleobase analogue
gccgauccauacugcggaauu

SEQ ID NO: 119 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-Methyl nucleotide
  modified_base (6)..(6): 2′-O-Methyl nucleotide
  modified_base (13)..(13): 2′-O-Methyl nucleotide
  modified_base (14)..(14): 2′-O-Methyl nucleotide
  modified_base (16)..(16): 2′-O-Methyl nucleotide
  modified_base (17)..(17): 2′-O-Methyl nucleotide
  modified_base (21)..(21): Unlocked nucleobase analogue
gccgauccauacugcggaauu

SEQ ID NO: 120 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-Methyl nucleotide
  modified_base (6)..(6): 2′-O-Methyl nucleotide
  modified_base (13)..(13): 2′-O-Methyl nucleotide
  modified_base (14)..(14): 2′-O-Methyl nucleotide
  modified_base (17)..(17): 2′-O-Methyl nucleotide
  modified_base (21)..(21): Unlocked nucleobase analogue
gccgauccauacugcggaauu

SEQ ID NO: 121 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-Methyl nucleotide
  modified_base (6)..(6): 2′-O-Methyl nucleotide
  modified_base (13)..(13): 2′-O-Methyl nucleotide
  modified_base (14)..(14): 2′-O-Methyl nucleotide
  modified_base (17)..(17): 2′-O-Methyl nucleotide
  modified_base (20)..(20): Unlocked nucleobase analogue
  modified_base (21)..(21): Unlocked nucleobase analogue
gccgauccauacugcggaauu

SEQ ID NO: 122 - RNA, 21 nt, Artificial Sequence
  Description of Artificial Sequence Synthetic oligonucleotide
  modified_base (1)..(1): Unlocked nucleobase analogue
  modified_base (3)..(3): 2′-O-Methyl nucleotide
  modified_base (6)..(6): 2′-O-Methyl nucleotide
  modified_base (12)..(12): 2′-O-Methyl nucleotide
  modified_base (14)..(14): 2′-O-Methyl nucleotide
  modified_base (21)..(21): Unlocked nucleobase analogue
gcucaguuuacuagugccauu

SEQ ID NO: 123 - RNA, 21 nt, Artificial Sequence
  Description of Artificial Sequence Synthetic oligonucleotide
  modified_base (1)..(1): Unlocked nucleobase analogue
  modified_base (2)..(2): 2′-O-Methyl nucleotide
  modified_base (3)..(3): 2′-O-Methyl nucleotide
  modified_base (6)..(6): 2′-O-Methyl nucleotide
  modified_base (12)..(12): 2′-O-Methyl nucleotide
  modified_base (20)..(20): Unlocked nucleobase analogue
  modified_base (21)..(21): Unlocked nucleobase analogue
cuggcucaguuuacuaguguu

SEQ ID NO: 124 - RNA, 21 nt, Artificial Sequence
  Description of Artificial Sequence Synthetic oligonucleotide
  modified_base (7)..(7): 2′-O-Methyl nucleotide
  modified_base (15)..(15): 2′-O-Methyl nucleotide
  modified_base (20)..(20): Unlocked nucleobase analogue
  modified_base (21)..(21): Unlocked nucleobase analogue
acaaacgggcaacauaccuuu

SEQ ID NO: 125 - RNA, 21 nt, Artificial Sequence
  Description of Artificial Sequence Synthetic oligonucleotide
  modified_base (10)..(10): 2′-O-Methyl nucleotide
  modified_base (18)..(18): 2′-O-Methyl nucleotide
uggcacuaguaaacugagcuu

SEQ ID NO: 126 - RNA, 21 nt, Artificial Sequence
  Description of Artificial Sequence Synthetic oligonucleotide
  modified_base (12)..(12): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
ugaagcgaagugcacacgguu

SEQ ID NO: 127 - RNA, 21 nt, Artificial Sequence
  Description of Artificial Sequence Synthetic oligonucleotide
  modified_base (10)..(10): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
uggcacuaguaaacugagcuu

SEQ ID NO: 128 - RNA, 21 nt, Artificial Sequence
  Description of Artificial Sequence Synthetic oligonucleotide
  modified_base (12)..(12): 2′-O-Methyl nucleotide
  modified_base (18)..(18): 2′-O-Methyl nucleotide
ugaagcgaagugcacacgguu

SEQ ID NO: 129 - RNA, 21 nt, Artificial Sequence
  Description of Artificial Sequence Synthetic oligonucleotide
  modified_base (6)..(6): 2′-O-Methyl nucleotide
  modified_base (13)..(13): 2′-O-Methyl nucleotide
  modified_base (15)..(15): 2′-O-Methyl nucleotide
cacuaguaaacugagccaguu

SEQ ID NO: 130 - RNA, 21 nt, Artificial Sequence
  Description of Artificial Sequence Synthetic oligonucleotide
  modified_base (12)..(12): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
ugaagcgaagugcacacgguu

SEQ ID NO: 131 - RNA, 21 nt, Artificial Sequence
  Description of Artificial Sequence Synthetic oligonucleotide
  modified_base (15)..(15): 2′-O-Methyl nucleotide
  modified_base (20)..(20): Unlocked nucleobase analogue
  modified_base (21)..(21): Unlocked nucleobase analogue
uggcacuaguaaacugagcuu

SEQ ID NO: 132 - RNA, 21 nt, Artificial Sequence
  Description of Artificial Sequence Synthetic oligonucleotide
  modified_base (7)..(7): 2′-O-Methyl nucleotide
  modified_base (15)..(15): 2′-O-Methyl nucleotide
  modified_base (19)..(19): 2′-O-Methyl nucleotide
  modified_base (20)..(20): Unlocked nucleobase analogue
  modified_base (21)..(21): Unlocked nucleobase analogue
acaaacgggcaacauaccuuu

SEQ ID NO: 133 - RNA, 21 nt, Artificial Sequence
  Description of Artificial Sequence Synthetic oligonucleotide
  modified_base (8)..(8): 2′-O-Methyl nucleotide
  modified_base (13)..(13): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
uuccgcaguauggaucggcuu

SEQ ID NO: 134 - RNA, 21 nt, Artificial Sequence
  Description of Artificial Sequence Synthetic oligonucleotide
  modified_base (8)..(8): 2′-O-Methyl nucleotide
  modified_base (13)..(13): 2′-O-Methyl nucleotide
  modified_base (18)..(18): 2′-O-Methyl nucleotide
uuccgcaguauggaucggcuu

SEQ ID NO: 135 - RNA, 21 nt, Artificial Sequence
  Description of Artificial Sequence Synthetic oligonucleotide
  modified_base (8)..(8): 2′-O-Methyl nucleotide
  modified_base (13)..(13): 2′-O-Methyl nucleotide
  modified_base (18)..(18): 2′-O-Methyl nucleotide
uuccgcaguauggaucggcuu

SEQ ID NO: 136 - RNA, 21 nt, Artificial Sequence
  Description of Artificial Sequence Synthetic oligonucleotide
  modified_base (8)..(8): 2′-O-Methyl nucleotide
  modified_base (13)..(13): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
uuccgcaguauggaucggcuu

SEQ ID NO: 137 - RNA, 21 nt, Artificial Sequence
  Description of Artificial Sequence Synthetic oligonucleotide
  modified_base (15)..(15): 2′-O-Methyl nucleotide
uggcacuaguaaacugagcuu

SEQ ID NO: 138 - RNA, 21 nt, Artificial Sequence
  Description of Artificial Sequence Synthetic oligonucleotide
  modified_base (15)..(15): 2′-O-Methyl nucleotide
  modified_base (20)..(20): Unlocked nucleobase analogue
  modified_base (21)..(21): Unlocked nucleobase analogue
cacuaguaaacugagccaguu

SEQ ID NO: 139 - RNA, 21 nt, Artificial Sequence
  Description of Artificial Sequence Synthetic oligonucleotide
  modified_base (1)..(1): Unlocked nucleoside analog
  modified_base (5)..(5): 2′-O-Methyl nucleotide
  modified_base (6)..(6): 2′-O-Methyl nucleotide
  modified_base (12)..(12): 2′-O-Methyl nucleotide
  modified_base (16)..(16): 2′-O-Methyl nucleotide
  modified_base (17)..(17): 2′-O-Methyl nucleotide
  modified_base (20)..(20): Unlocked nucleoside analog
  modified_base (21)..(21): Unlocked nucleoside analog
ccgugugcacuucgcuucauu

SEQ ID NO: 140 - RNA, 21 nt, Artificial Sequence
  Description of Artificial Sequence Synthetic oligonucleotide
  modified_base (3)..(3): 2′-O-Methyl nucleotide
  modified_base (5)..(5): 2′-O-Methyl nucleotide
  modified_base (6)..(6): 2′-O-Methyl nucleotide
  modified_base (12)..(12): 2′-O-Methyl nucleotide
  modified_base (14)..(14): 2′-O-Methyl nucleotide
  modified_base (16)..(16): 2′-O-Methyl nucleotide
  modified_base (17)..(17): 2′-O-Methyl nucleotide
ccgugugcacuucgcuucauu

SEQ ID NO: 141 - RNA, 21 nt, Artificial Sequence
  Description of Artificial Sequence Synthetic oligonucleotide
  modified_base (1)..(1): Unlocked nucleoside analog
  modified_base (3)..(3): 2′-O-Methyl nucleotide
  modified_base (5)..(5): 2′-O-Methyl nucleotide
  modified_base (6)..(6): 2′-O-Methyl nucleotide
  modified_base (12)..(12): 2′-O-Methyl nucleotide
  modified_base (14)..(14): 2′-O-Methyl nucleotide
  modified_base (16)..(16): 2′-O-Methyl nucleotide
  modified_base (17)..(17): 2′-O-Methyl nucleotide
  modified_base (20)..(20): Unlocked nucleoside analog
  modified_base (21)..(21): Unlocked nucleoside analog
ccgugugcacuucgcuucauu

SEQ ID NO: 142 - RNA, 21 nt, Artificial Sequence
  Description of Artificial Sequence Synthetic oligonucleotide
  modified_base (3)..(3): 2′-O-Methyl nucleotide
  modified_base (4)..(4): 2′-O-Methyl nucleotide
  modified_base (6)..(6): 2′-O-Methyl nucleotide
  modified_base (10)..(10): 2′-O-Methyl nucleotide
  modified_base (13)..(13): 2′-O-Methyl nucleotide
  modified_base (14)..(14): 2′-O-Methyl nucleotide
  modified_base (17)..(17): 2′-O-Methyl nucleotide
gccgauccauacugcggaauu

SEQ ID NO: 143 - RNA, 21 nt, Artificial Sequence
  Description of Artificial Sequence Synthetic oligonucleotide
  modified_base (1)..(1): Unlocked nucleoside analog
  modified_base (3)..(3): 2′-O-Methyl nucleotide
  modified_base (4)..(4): 2′-O-Methyl nucleotide
  modified_base (6)..(6): 2′-O-Methyl nucleotide
  modified_base (10)..(10): 2′-O-Methyl nucleotide
  modified_base (13)..(13): 2′-O-Methyl nucleotide
  modified_base (14)..(14): 2′-O-Methyl nucleotide
  modified_base (17)..(17): 2′-O-Methyl nucleotide
  modified_base (21)..(21): Unlocked nucleoside analog
gccgauccauacugcggaauu

SEQ ID NO: 144 - RNA, 21 nt, Artificial Sequence
  Description of Artificial Sequence Synthetic oligonucleotide
  modified_base (1)..(1): Unlocked nucleoside analog
  modified_base (2)..(2): 2′-O-Methyl nucleotide
  modified_base (3)..(3): 2′-O-Methyl nucleotide
  modified_base (4)..(4): 2′-O-Methyl nucleotide
  modified_base (15)..(15): 2′-O-Methyl nucleotide
  modified_base (17)..(17): 2′-O-Methyl nucleotide
  modified_base (21)..(21): Unlocked nucleoside analog
cuggcucaguuuacuaguguu

SEQ ID NO: 145 - RNA, 21 nt, Artificial Sequence
  Description of Artificial Sequence Synthetic oligonucleotide
  modified_base (2)..(2): 2′-O-Methyl nucleotide
  modified_base (3)..(3): 2′-O-Methyl nucleotide
  modified_base (4)..(4): 2′-O-Methyl nucleotide
  modified_base (6)..(6): 2′-O-Methyl nucleotide
  modified_base (10)..(10): 2′-O-Methyl nucleotide
  modified_base (12)..(12): 2′-O-Methyl nucleotide
  modified_base (14)..(14): 2′-O-Methyl nucleotide
  modified_base (15)..(15): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
cuggcucaguuuacuaguguu

SEQ ID NO: 146 - RNA, 21 nt, Artificial Sequence
  Description of Artificial Sequence Synthetic oligonucleotide
  modified_base (1)..(1): Unlocked nucleoside analog
  modified_base (2)..(2): 2′-O-Methyl nucleotide
  modified_base (3)..(3): 2′-O-Methyl nucleotide
  modified_base (4)..(4): 2′-O-Methyl nucleotide
  modified_base (6)..(6): 2′-O-Methyl nucleotide
  modified_base (10)..(10): 2′-O-Methyl nucleotide
  modified_base (12)..(12): 2′-O-Methyl nucleotide
  modified_base (14)..(14): 2′-O-Methyl nucleotide
  modified_base (15)..(15): 2′-O-Methyl nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  modified_base (21)..(21): Unlocked nucleoside analog
cuggcucaguuuacuaguguu

SEQ ID NO: 147 - RNA, 21 nt, Artificial Sequence
  Description of Artificial Sequence Synthetic oligonucleotide
  modified_base (2)..(2): 2′-O-Methyl nucleotide
  modified_base (4)..(4): 2′-O-Methyl nucleotide
  modified_base (6)..(6): 2′-O-Methyl nucleotide
  modified_base (8)..(8): 2′-O-Methyl nucleotide
  modified_base (10)..(10): 2′-O-Methyl nucleotide
  modified_base (12)..(12): 2′-O-Methyl nucleotide
  modified_base (14)..(14): 2′-O-Methyl nucleotide
accucugccuaaucaucucuu

SEQ ID NO: 148 - RNA, 21 nt, Artificial Sequence
  Description of Artificial Sequence Synthetic oligonucleotide
  modified_base (1)..(1): Unlocked nucleoside analog
  modified_base (2)..(2): 2′-O-Methyl nucleotide
  modified_base (4)..(4): 2′-O-Methyl nucleotide
  modified_base (6)..(6): 2′-O-Methyl nucleotide
  modified_base (8)..(8): 2′-O-Methyl nucleotide
  modified_base (10)..(10): 2′-O-Methyl nucleotide
  modified_base (12)..(12): 2′-O-Methyl nucleotide
  modified_base (14)..(14): 2′-O-Methyl nucleotide
  modified_base (20)..(20): Unlocked nucleoside analog
accucugccuaaucaucucuu

SEQ ID NO: 149 - RNA, 21 nt, Artificial Sequence
  Description of Artificial Sequence Synthetic oligonucleotide
  modified_base (12)..(12): 2′-O-Methyl nucleotide
  modified_base (18)..(18): 2′-O-Methyl nucleotide
  modified_base (20)..(20): Unlocked nucleoside analog
  modified_base (21)..(21): Unlocked nucleoside analog
ugaagcgaagugcacacgguu

SEQ ID NO: 150 - RNA, 21 nt, Artificial Sequence
  Description of Artificial Sequence Synthetic oligonucleotide
  modified_base (5)..(5): 2′-O-Methyl nucleotide
  modified_base (7)..(7): 2′-O-Methyl nucleotide
  modified_base (11)..(11): 2′-O-Methyl nucleotide
  modified_base (12)..(12): 2′-O-Methyl nucleotide
  modified_base (15)..(15): 2′-O-Methyl nucleotide
  modified_base (17)..(17): 2′-O-Methyl nucleotide
  modified_base (18)..(18): 2′-O-Methyl nucleotide
ugaagcgaagugcacacgguu

SEQ ID NO: 151 - RNA, 21 nt, Artificial Sequence
  Description of Artificial Sequence Synthetic oligonucleotide
  modified_base (5)..(5): 2′-O-Methyl nucleotide
  modified_base (7)..(7): 2′-O-Methyl nucleotide
  modified_base (11)..(11): 2′-O-Methyl nucleotide
  modified_base (12)..(12): 2′-O-Methyl nucleotide
  modified_base (15)..(15): 2′-O-Methyl nucleotide
  modified_base (17)..(17): 2′-O-Methyl nucleotide
  modified_base (18)..(18): 2′-O-Methyl nucleotide
  modified_base (20)..(20): Unlocked nucleoside analog
  modified_base (21)..(21): Unlocked nucleoside analog
ugaagcgaagugcacacgguu

SEQ ID NO: 152 - RNA, 21 nt, Artificial Sequence
  Description of Artificial Sequence Synthetic oligonucleotide
  modified_base (8)..(8): 2′-O-Methyl nucleotide
  modified_base (13)..(13): 2′-O-Methyl nucleotide
  modified_base (18)..(18): 2′-O-Methyl nucleotide
uuccgcaguauggaucggcuu

SEQ ID NO: 153 - RNA, 21 nt, Artificial Sequence
  Description of Artificial Sequence Synthetic oligonucleotide
  modified_base (8)..(8): 2′-O-Methyl nucleotide
  modified_base (13)..(13): 2′-O-Methyl nucleotide
  modified_base (18)..(18): 2′-O-Methyl nucleotide
uuccgcaguauggaucggcuu

SEQ ID NO: 154 - RNA, 21 nt, Artificial Sequence
  Description of Artificial Sequence Synthetic oligonucleotide
  modified_base (6)..(6): 2′-O-Methyl nucleotide
  modified_base (13)..(13): 2′-O-Methyl nucleotide
  modified_base (15)..(15): 2′-O-Methyl nucleotide
cacuaguaaacugagccaguu

SEQ ID NO: 155 - RNA, 21 nt, Artificial Sequence
  Description of Artificial Sequence Synthetic oligonucleotide
  modified_base (6)..(6): 2′-O-Methyl nucleotide
  modified_base (7)..(7): 2′-O-Methyl nucleotide
  modified_base (11)..(11): 2′-O-Methyl nucleotide
  modified_base (13)..(13): 2′-O-Methyl nucleotide
  modified_base (15)..(15): 2′-O-Methyl nucleotide
  modified_base (17)..(17): 2′-O-Methyl nucleotide
cacuaguaaacugagccaguu

SEQ ID NO: 156 - RNA, 21 nt, Artificial Sequence
  Description of Artificial Sequence Synthetic oligonucleotide
  modified_base (6)..(6): 2′-O-Methyl nucleotide
  modified_base (7)..(7): 2′-O-Methyl nucleotide
  modified_base (11)..(11): 2′-O-Methyl nucleotide
  modified_base (13)..(13): 2′-O-Methyl nucleotide
  modified_base (15)..(15): 2′-O-Methyl nucleotide
  modified_base (17)..(17): 2′-O-Methyl nucleotide
cacuaguaaacugagccaguu

SEQ ID NO: 157 - RNA, 21 nt, Artificial Sequence
  Description of Artificial Sequence Synthetic oligonucleotide
  modified_base (7)..(7): 2′-O-Methyl nucleotide
  modified_base (9)..(9): 2′-O-Methyl nucleotide
  modified_base (13)..(13): 2′-O-Methyl nucleotide
  modified_base (15)..(15): 2′-O-Methyl nucleotide
  modified_base (17)..(17): 2′-O-Methyl nucleotide
gagaugauuaggcagagguuu

SEQ ID NO: 158 - RNA, 21 nt, Artificial Sequence
  Description of Artificial Sequence Synthetic oligonucleotide
  modified_base (7)..(7): 2′-O-Methyl nucleotide
  modified_base (9)..(9): 2′-O-Methyl nucleotide
  modified_base (13)..(13): 2′-O-Methyl nucleotide
  modified_base (15)..(15): 2′-O-Methyl nucleotide
  modified_base (17)..(17): 2′-O-Methyl nucleotide
  modified_base (20)..(20): Unlocked nucleoside analog
gagaugauuaggcagagguuu

SEQ ID NO: 159 - DNA, 21 bp, Artificial Sequence
  Description of Artificial Sequence Synthetic primer
gacaaacgggcaacatacctt

SEQ ID NO: 160 - DNA, 20 bp, Artificial Sequence
  Description of Artificial Sequence Synthetic primer
gtgtctgcggcgttttatca

SEQ ID NO: 161 - DNA, 28 bp, Artificial Sequence
  Description of Artificial Sequence Synthetic probe
cctctkcatcctgctgctatgcctcatc

SEQ ID NO: 162 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
ugcacuucgcuucaccu

SEQ ID NO: 163 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
gugcacuucgcuucaca

SEQ ID NO: 164 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
aggugaagcgaagugcacacgu

SEQ ID NO: 165 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
ugugaagcgaagugcacacggu

SEQ ID NO: 166 - RNA, 21 nt, Artificial Sequence
  Description of Artificial Sequence Synthetic oligonucleotide
  modified_base (1)..(1): Unlocked nucleobase analogue
  modified_base (5)..(5): 2′-O-Methyl nucleotide
  modified_base (6)..(6): 2′-O-Methyl nucleotide
  modified_base (12)..(12): 2′-O-Methyl nucleotide
  modified_base (16)..(16): 2′-O-Methyl nucleotide
  modified_base (17)..(17): 2′-O-Methyl nucleotide
  modified_base (20)..(20): Unlocked nucleobase analogue
  modified_base (21)..(21): Unlocked nucleobase analogue
ccgugugcacuucgcuucauu

SEQ ID NO: 167 - RNA, 21 nt, Artificial Sequence
  Description of Artificial Sequence Synthetic oligonucleotide
  modified_base (1)..(1): Unlocked nucleobase analogue
  modified_base (2)..(2): 2′-O-Methyl nucleotide
  modified_base (3)..(3): 2′-O-Methyl nucleotide
  modified_base (4)..(4): 2′-O-Methyl nucleotide
  modified_base (15)..(15): 2′-O-Methyl nucleotide
  modified_base (17)..(17): 2′-O-Methyl nucleotide
  modified_base (21)..(21): Unlocked nucleobase analogue
cuggcucaguuuacuaguguu

SEQ ID NO: 168 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-Methyl nucleotide
  modified_base (6)..(6): 2′-O-Methyl nucleotide
  modified_base (13)..(13): 2′-O-Methyl nucleotide
  modified_base (14)..(14): 2′-O-Methyl nucleotide
  modified_base (17)..(17): 2′-O-Methyl nucleotide
  modified_base (21)..(21): Unlocked nucleobase analogue
gccgauccauacugcggaauu

SEQ ID NO: 169 - RNA, 21 nt, Artificial Sequence
  Description of Artificial Sequence Synthetic oligonucleotide
  modified_base (12)..(12): 2′-O-Methyl nucleotide
  modified_base (18)..(18): 2′-O-Methyl nucleotide
  modified_base (20)..(20): Unlocked nucleobase analogue
  modified_base (21)..(21): Unlocked nucleobase analogue
ugaagcgaagugcacacgguu

SEQ ID NO: 170 - RNA, 21 nt, Artificial Sequence
  Description of Artificial Sequence Synthetic oligonucleotide
  modified_base (6)..(6): 2′-O-Methyl nucleotide
  modified_base (13)..(13): 2′-O-Methyl nucleotide
  modified_base (15)..(15): 2′-O-Methyl nucleotide
cacuaguaaacugagccaguu

SEQ ID NO: 171 - RNA, 21 nt, Artificial Sequence
  Description of Artificial Sequence Synthetic oligonucleotide
  modified_base (8)..(8): 2′-O-Methyl nucleotide
  modified_base (13)..(13): 2′-O-Methyl nucleotide
  modified_base (18)..(18): 2′-O-Methyl nucleotide
uuccgcaguauggaucggcuu

SEQ ID NO: 172 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
ugcacuucgcuucaccu

SEQ ID NO: 173 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
gugcacuucgcuucaca

SEQ ID NO: 174 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
aggugaagcgaagugcacacgu

SEQ ID NO: 175 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
ugugaagcgaagugcacacggu

SEQ ID NO: 176 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
ugcacuucgcuucaccu

SEQ ID NO: 177 - RNA, 17 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-O-Methyl nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-Fluoro nucleotide
  modified_base (6)..(6): 2′-Fluoro nucleotide
  modified_base (7)..(7): 2′-Fluoro nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-O-Methyl nucleotide
  modified_base (15)..(15): 2′-O-Methyl nucleotide
  modified_base (16)..(16): 2′-O-Methyl nucleotide
  modified_base (17)..(17): 2′-O-Methyl nucleotide
gugcacuucgcuucaca

SEQ ID NO: 178 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
aggugaagcgaagugcacacgu

SEQ ID NO: 179 - RNA, 22 nt, Artificial Sequence
  Description of Artificial Sequence Synthetic oligonucleotide
  modified_base (1)..(1): 2′-O-Methyl nucleotide
  misc_feature (1)..(2): Phosphorothioate linker
  modified_base (2)..(2): 2′-Fluoro nucleotide
  misc_feature (2)..(3): Phosphorothioate linker
  modified_base (3)..(3): 2′-O-Methyl nucleotide
  modified_base (4)..(4): 2′-O-Methyl nucleotide
  modified_base (5)..(5): 2′-O-Methyl nucleotide
  modified_base (6)..(6): 2′-O-Methyl nucleotide
  modified_base (7)..(7): 2′-O-Methyl nucleotide
  modified_base (8)..(8): 2′-O-Methyl nucleotide
  modified_base (9)..(9): 2′-O-Methyl nucleotide
  modified_base (10)..(10): 2′-O-Methyl nucleotide
  modified_base (11)..(11): 2′-O-Methyl nucleotide
  modified_base (12)..(12): 2′-O-Methyl nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-O-Methyl nucleotide
  modified_base (19)..(19): 2′-O-Methyl nucleotide
  misc_feature (19)..(20): Phosphorothioate linker
  modified_base (20)..(20): 2′-O-Methyl nucleotide
  misc_feature (20)..(21): Phosphorothioate linker
  modified_base (21)..(21): 2′-O-Methyl nucleotide
ugugaagcgaagugcacacggu

(19) United States
(12) Patent Application Publication
(10) Pub. No.: US 2022/0168430 A1
(43) Pub. Date: Jun. 2, 2022

(54) THERAPEUTIC METHODS FOR TREATING HEPATITIS B

(71) Applicant: ARBUTUS BIOPHARMA CORPORATION, Warminster, PA (US)

(72) Inventors: Andrzej ARDZINSKI, Burnaby (CA); Andrea CUCONATI, Burnaby (CA); Amy C. H. LEE, Burnaby (CA); Nagraj MANI, Burnaby (CA); Cornelis A. RIJNBRAND, Burnaby (CA); Michael J. SOFIA, Burnaby (CA); Emily P. THI, Burnaby (CA)

(73) Assignee: ARBUTUS BIOPHARMA CORPORATION, Warminster, PA (US)

(21) Appl. No.: 17/440,480

(22) PCT Filed: Mar. 19, 2020
(86) PCT No.: PCT/US2020/023657

§ 371 Date: Sep. 17, 2021

Related U.S. Application Data
(60) Provisional application No. 62/900,185, filed on Sep. 13, 2019.
(60) Provisional application No. 62/825,517, filed on Mar. 28, 2019.
(60) Provisional application No. 62/821,099, filed on Mar. 20, 2019.

(51) Int. Cl. A61K 47/55; A61K 47/60; A61K 38/21; A61K 31/675; A61K 31/522; A61K 31/4196; A61K 31/4375; A61K 31/7088; A61K 47/54; A61P 31/20

(52) U.S. Cl. CPC A61K 47/55; A61K 47/60; A61K 38/212; A61K 31/675; A61P 31/20; A61K 31/4196; A61K 31/4375; A61K 31/7088; A61K 47/549; A61K 31/522

(57) ABSTRACT

The invention provides therapeutic combinations and therapeutic methods that are useful for treating Hepatitis B and Hepatitis D.

### CROSS-REFERENCE TO RELATED APPLICATIONS

This patent application claims the benefit of priority of U.S. application Ser. No. 62/821,099, filed Mar. 20, 2019, U.S. application Ser. No. 62/825,517, filed Mar. 28, 2019, and U.S. application Ser. No. 62/900,185, filed Sep. 13, 2019, which applications are herein incorporated by reference.

### BACKGROUND

Hepatitis B virus (abbreviated as “HBV”) is a member of the Hepadnavirus family. The virus particle (sometimes referred to as a virion) includes an outer lipid envelope and an icosahedral nucleocapsid core composed of protein. The nucleocapsid encloses the viral DNA and a DNA polymerase that has reverse transcriptase activity. The outer envelope contains embedded proteins that are involved in viral binding of, and entry into, susceptible cells, typically liver hepatocytes. In addition to the infectious viral particles, filamentous and spherical bodies lacking a core can be found in the serum of infected individuals. These particles are not infectious and are composed of the lipid and protein that forms part of the surface of the virion, which is called the surface antigen (HBsAg), and is produced in excess during the life cycle of the virus.

The genome of HBV is made of circular DNA, but it is unusual because the DNA is not fully double-stranded. One end of the full-length strand is linked to the viral DNA polymerase.

The genome is 3020-3320 nucleotides long (for the full-length strand) and 1700-2800 nucleotides long (for the shorter strand). The negative-sense (non-coding) is complementary to the viral mRNA. The viral DNA is found in the nucleus soon after infection of the cell. There are four known genes encoded by the genome, called C, X, P, and S. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The HBsAg gene is one long open reading frame but contains three in frame “start” (ATG) codons that divide the gene into three sections, pre-S1, pre-S2, and S. Because of the multiple start codons, polypeptides of three different sizes called large, middle, and small are produced. The function of the protein coded for by gene X is not fully understood but it is associated with the development of liver cancer. Replication of HBV is a complex process. Although replication takes place in the liver, the virus spreads to the blood where viral proteins and antibodies against them are found in infected people. The structure, replication and biology of HBV is reviewed in D. Glebe and C. M. Bremer, Seminars in Liver Disease, Vol. 33, No. 2, pages 103-112 (2013).

Infection of humans with HBV can cause an infectious inflammatory illness of the liver. Infected individuals may not exhibit symptoms for many years. It is estimated that about a third of the world population has been infected at one point in their lives, including 350 million who are chronic carriers.

The virus is transmitted by exposure to infectious blood or body fluids. Perinatal infection can also be a major route of infection. The acute illness causes liver inflammation, vomiting, jaundice, and possibly death. Chronic hepatitis B may eventually cause cirrhosis and liver cancer.

Although most people who are infected with HBV clear the infection through the action of their immune system, some infected people suffer an aggressive course of infection (fulminant hepatitis); while others are chronically infected thereby increasing their chance of liver disease. Several medications are currently approved for treatment of HBV infection, but infected individuals respond with various degrees of success to these medications, and none of these medications clear the virus from the infected person.

Hepatitis D virus (HDV) is a small circular enveloped RNA virus that can propagate only in the presence of the hepatitis B virus (HBV). Specifically, HDV requires the HBV surface antigen protein to propagate itself. Infection with both HBV and HDV results in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B virus, hepatitis D has the highest mortality rate of all the hepatitis infections. The routes of transmission of HDV are similar to those for HBV. Infection is largely restricted to persons at high risk of HBV infection, particularly injecting drug users and persons receiving clotting factor concentrates.

Thus, there is a continuing need for compositions and methods for the treatment of HBV infection in animals (e.g. humans), as well as for the treatment of HBV/HDV infection in animals (e.g. humans).

### SUMMARY

The present invention provides therapeutic combinations and therapeutic methods that are useful for treating viral infections such as HBV and HDV. The Examples presented herein disclose the results of combination studies using agents having differing mechanisms of action against HBV. Accordingly, certain embodiments of the invention provide a combination described herein.

### DETAILED DESCRIPTION

Described herein are therapeutic combinations and therapeutic methods that are useful for treating viral infections such as HBV and HDV. One embodiment provides methods of ameliorating at least one symptom of HBV infection in a human subject infected with HBV, the method comprising the steps of:
(a) administering to the human subject a GalNAc-siRNA conjugate, wherein the siRNA portion of the conjugate targets a portion of the HBV genome; and
(b) administering to the subject at least one anti-HBV agent selected from the group consisting of: an RNA destabilizer; a capsid inhibitor; a reverse transcriptase inhibitor; an immunostimulator; a cccDNA formation inhibitor; and an oligomeric nucleotide targeted to the Hepatitis B genome.

In certain embodiments, the method comprises administering to the subject an RNA destabilizer.

In certain embodiments, the method comprises administering to the subject a capsid inhibitor.

In certain embodiments, the method comprises administering to the subject a reverse transcriptase inhibitor.

In certain embodiments, the method comprises administering to the subject an immunostimulator.

In certain embodiments, the method comprises administering to the subject a cccDNA formation inhibitor.

In certain embodiments, the method comprises administering to the subject an oligomeric nucleotide targeted to the Hepatitis B genome.

In certain embodiments, the GalNAc-siRNA conjugate is administered subcutaneously.

In certain embodiments, the anti-HBV agent of step (b) is administered orally.

In certain embodiments, the anti-HBV agent of step (b) is administered orally in pill form.

In certain embodiments, the reverse transcriptase inhibitor is a nucleoside analogue HBV reverse transcriptase inhibitor.

In certain embodiments, the GalNAc-siRNA conjugate is a compound of formula (V), as described in Examples 1-4, or a salt thereof.

In certain embodiments, the RNA destabilizer is a compound of formula (VI), as described in Examples 1-4, or a salt thereof.

In certain embodiments, the capsid inhibitor is a compound of formula (VII), as described in Examples 1-4, or a salt thereof.

In certain embodiments, the immunostimulator is a pegylated interferon (PEG-IFN).

In certain embodiments, the immunostimulator is pegylated interferon alpha 2a (PEG-IFNα2a).

In certain embodiments, the reverse transcriptase inhibitor is tenofovir alafenamide fumarate (TAF).

In certain embodiments, the GalNAc-siRNA conjugate is administered simultaneously with the anti-HBV agent of step (b).

In certain embodiments, the GalNAc-siRNA conjugate and the anti-HBV agent of step (b) are administered sequentially.

In certain embodiments, the GalNAc-siRNA conjugate is administered prior to the administration of the anti-HBV agent of step (b).

In certain embodiments, the GalNAc-siRNA conjugate is administered after the administration of the anti-HBV agent of step (b).

In certain embodiments, the method further comprises administering at least one additional therapeutic agent to the subject.

One embodiment provides methods of ameliorating at least one symptom of HDV infection in a human subject infected with HDV, the method comprising the steps of:

(a) administering to the human subject a GalNAc-siRNA conjugate, wherein the siRNA portion of the conjugate targets a portion of the HBV genome; and

(b) administering to the subject at least one anti-HBV agent selected from the group consisting of: an RNA destabilizer; a capsid inhibitor; a reverse transcriptase inhibitor; an immunostimulator; a cccDNA formation inhibitor; and an oligomeric nucleotide targeted to the Hepatitis B genome.

The use of a combination of a GalNAc-siRNA conjugate, wherein the siRNA portion of the conjugate targets a portion of the HBV genome, and at least one anti-HBV agent selected from the group consisting of: an RNA destabilizer; a capsid inhibitor; a reverse transcriptase inhibitor; an immunostimulator; a cccDNA formation inhibitor; and an oligomeric nucleotide targeted to the Hepatitis B genome, to ameliorate at least one symptom of HBV infection in a human subject, is also provided.

The use of a combination of a GalNAc-siRNA conjugate, wherein the siRNA portion of the conjugate targets a portion of the HBV genome, and at least one anti-HBV agent selected from the group consisting of: an RNA destabilizer; a capsid inhibitor; a reverse transcriptase inhibitor; an immunostimulator; a cccDNA formation inhibitor; and an oligomeric nucleotide targeted to the Hepatitis B genome, to treat HBV infection in a human subject, is also provided.

The use of a combination of a GalNAc-siRNA conjugate, wherein the siRNA portion of the conjugate targets a portion of the HBV genome, and at least one anti-HBV agent selected from the group consisting of: an RNA destabilizer; a capsid inhibitor; a reverse transcriptase inhibitor; an immunostimulator; a cccDNA formation inhibitor; and an oligomeric nucleotide targeted to the Hepatitis B genome, to treat HDV infection in a human subject, is also provided.

In one embodiment the invention provides a pharmaceutical composition that comprises a pharmaceutically acceptable carrier and at least two agents selected from the group consisting of:

a) a capsid inhibitor, wherein the capsid inhibitor is:

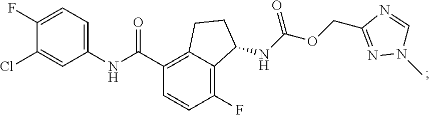 [SMILES: Cn1cnc(COC(=O)N[C@H]2CCc3c(C(=O)Nc4ccc(F)c(Cl)c4)ccc(F)c32)n1 ; ";"]

b) an RNA destabilizer, wherein the RNA destabilizer is:

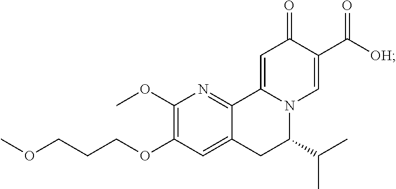 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and

d) oligomeric nucleotides targeted to the Hepatitis B genome.

In one embodiment the invention provides a pharmaceutical composition that comprises a pharmaceutically acceptable carrier and at least three agents selected from the group consisting of:
a) a capsid inhibitor, wherein the capsid inhibitor is:

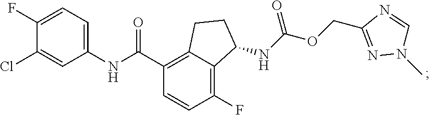 [SMILES: Cn1cnc(COC(=O)N[C@H]2CCc3c(C(=O)Nc4ccc(F)c(Cl)c4)ccc(F)c32)n1 ; ";"]

b) an RNA destabilizer, wherein the RNA destabilizer is:

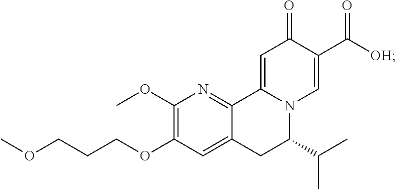 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and
d) oligomeric nucleotides targeted to the Hepatitis B genome.

In another embodiment the invention provides a kit comprising at least two agents selected from the group consisting of:
a) a capsid inhibitor, wherein the capsid inhibitor is:

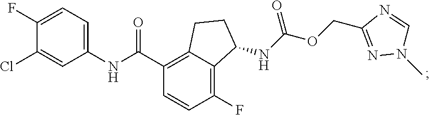 [SMILES: Cn1cnc(COC(=O)N[C@H]2CCc3c(C(=O)Nc4ccc(F)c(Cl)c4)ccc(F)c32)n1 ; ";"]

b) an RNA destabilizer, wherein the RNA destabilizer is:

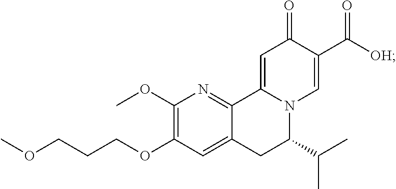 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and
d) oligomeric nucleotides targeted to the Hepatitis B genome;
   for use in combination to treat or prevent a viral infection, such as Hepatitis B.

In another embodiment the invention provides a kit comprising at least three agents selected from the group consisting of:
a) a capsid inhibitor, wherein the capsid inhibitor is:

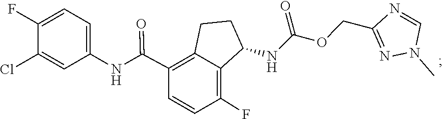 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

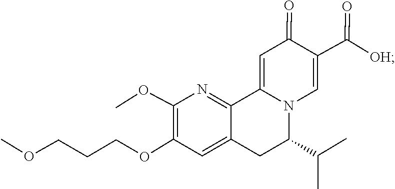 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and
d) oligomeric nucleotides targeted to the Hepatitis B genome;
   for use in combination to treat or prevent a viral infection, such as Hepatitis B.

In another embodiment the invention provides a kit comprising at least two agents selected from the group consisting of:
a) a capsid inhibitor, wherein the capsid inhibitor is:

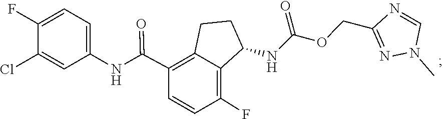 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

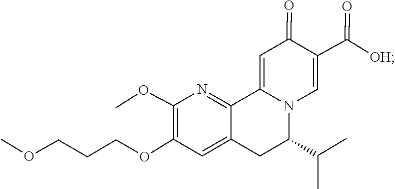 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and
d) oligomeric nucleotides targeted to the Hepatitis B genome;
   for use in combination to treat or prevent a viral infection, such as Hepatitis D.

In another embodiment the invention provides a kit comprising at least three agents selected from the group consisting of:
a) a capsid inhibitor, wherein the capsid inhibitor is:

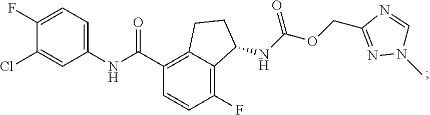 [SMILES: Cn1cnc(COC(=O)N[C@H]2CCc3c(C(=O)Nc4ccc(F)c(Cl)c4)ccc(F)c32)n1 ; ";"]

b) an RNA destabilizer, wherein the RNA destabilizer is:

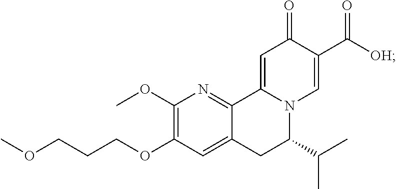 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and
d) oligomeric nucleotides targeted to the Hepatitis B genome;
   for use in combination to treat or prevent a viral infection, such as Hepatitis D.

In another embodiment the invention provides a method for treating Hepatitis B in an animal comprising administering to the animal, at least two agents selected from the group consisting of:

a) a capsid inhibitor, wherein the capsid inhibitor is:

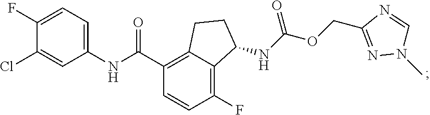 [SMILES: Cn1cnc(COC(=O)N[C@H]2CCc3c(C(=O)Nc4ccc(F)c(Cl)c4)ccc(F)c32)n1 ; ";"]

b) an RNA destabilizer, wherein the RNA destabilizer is:

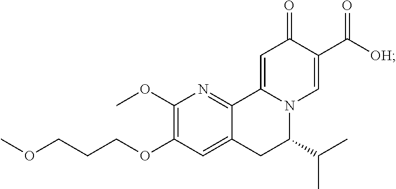 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and

d) oligomeric nucleotides targeted to the Hepatitis B genome.

In another embodiment the invention provides a method for treating Hepatitis B in an animal comprising administering to the animal, at least three agents selected from the group consisting of:

a) a capsid inhibitor, wherein the capsid inhibitor is:

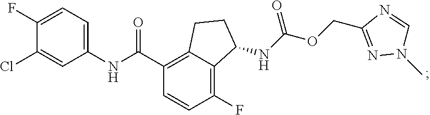 [SMILES: Cn1cnc(COC(=O)N[C@H]2CCc3c(C(=O)Nc4ccc(F)c(Cl)c4)ccc(F)c32)n1 ; ";"]

b) an RNA destabilizer, wherein the RNA destabilizer is:

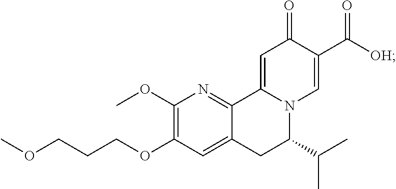 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and

d) oligomeric nucleotides targeted to the Hepatitis B genome.

In another embodiment the invention provides a method for treating Hepatitis D in an animal comprising administering to the animal, at least two agents selected from the group consisting of:

a) a capsid inhibitor, wherein the capsid inhibitor is:

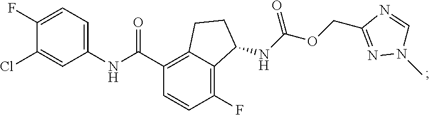 [SMILES: Cn1cnc(COC(=O)N[C@H]2CCc3c(C(=O)Nc4ccc(F)c(Cl)c4)ccc(F)c32)n1 ; ";"]

b) an RNA destabilizer, wherein the RNA destabilizer is:

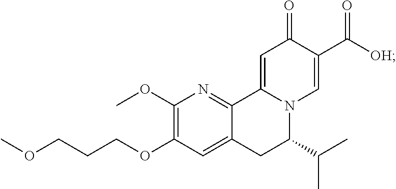 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and

d) oligomeric nucleotides targeted to the Hepatitis B genome.

In another embodiment the invention provides a method for treating Hepatitis D in an animal comprising administering to the animal, at least three agents selected from the group consisting of:

a) a capsid inhibitor, wherein the capsid inhibitor is:

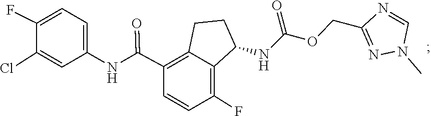 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

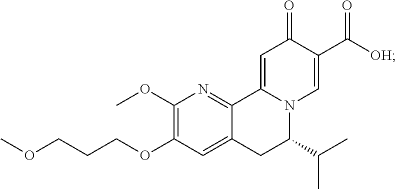 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and

d) oligomeric nucleotides targeted to the Hepatitis B genome.

Certain embodiments also provide a combination of at least two agents selected from the group consisting of:

a) a capsid inhibitor, wherein the capsid inhibitor is:

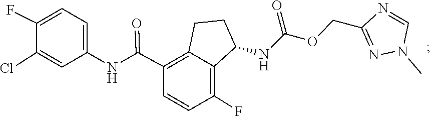 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

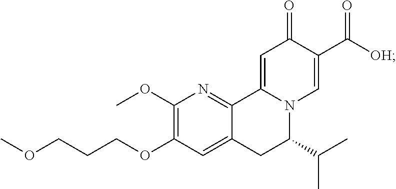 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and

d) oligomeric nucleotides targeted to the Hepatitis B genome, for use in treating Hepatitis B or Hepatitis D in an animal.

Certain embodiments also provide the use of a combination of at least two agents selected from the group consisting of:

a) a capsid inhibitor, wherein the capsid inhibitor is:

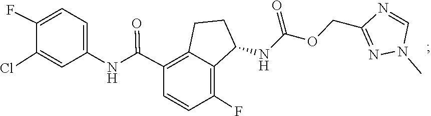 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

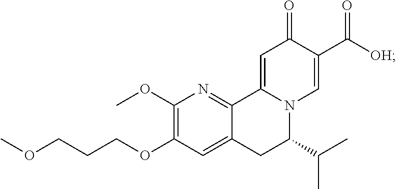 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and

d) oligomeric nucleotides targeted to the Hepatitis B genome, in the manufacture of a medicament for the treatment of Hepatitis B or Hepatitis D in an animal.

Administration of a compound as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Reverse Transcriptase Inhibitors

In certain embodiments, the reverse transcriptase inhibitor is a nucleoside analog.

In certain embodiments, the reverse transcriptase inhibitor is a nucleoside analog reverse-transcriptase inhibitor (NARTI or NRTI).

In certain embodiments, the reverse transcriptase inhibitor is a nucleoside analog inhibitor of HBV polymerase.

In certain embodiments, the reverse transcriptase inhibitor is a nucleotide analog reverse-transcriptase inhibitor (NtARTI or NtRTI).

In certain embodiments, the reverse transcriptase inhibitor is a nucleotide analog inhibitor of HBV polymerase.

The term reverse transcriptase inhibitor includes, but is not limited to: entecavir (ETV), clevudine, telbivudine, lamivudine, adefovir, tenofovir, tenofovir disoproxil, tenofovir alafenamide (TAF), tenofovir disoproxil fumarate (TDF), adefovir dipovoxil, (1R,2R,3R,5R)-3-(6-amino-9H-9-purinyl)-2-fluoro-5-(hydroxymethyl)-4-methylenecyclopentan-1-ol (described in U.S. Pat. No. 8,816,074), emtricitabine, abacavir, elvucitabine, ganciclovir, lobucavir, famciclovir, penciclovir, and amdoxovir.

The term reverse transcriptase inhibitor includes, but is not limited to: the reverse transcriptase inhibitor is entecavir (ETV), tenofovir disoproxil fumarate (TDF) or tenofovir alafenamide (TAF).

The term reverse transcriptase inhibitor includes, but is not limited to, entecavir, lamivudine, and (1R,2R,3R,5R)-3-(6-amino-9H-9-purinyl)-2-fluoro-5-(hydroxymethyl)-4-methylenecyclopentan-1-ol.

The term reverse transcriptase inhibitor includes, but is not limited to a covalently bound phosphoramidate or phosphonamidate moiety of the above-mentioned reverse transcriptase inhibitors, or as described in, for example, U.S. Pat. No. 8,816,074, US 2011/0245484 A1, and US 2008/0286230A1.

The term reverse transcriptase inhibitor includes, but is not limited to, nucleotide analogs that comprise a phosphoramidate moiety, such as, methyl ((((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((((1R,2R,3R,4R)-3-fluoro-2-hydroxy-5-methylene-4-(6-oxo-1,6-dihydro-9H-purin-9-yl)cyclopentyl)methoxy)(phenoxy)phosphoryl)-(D or L)-alaninate. Also included are the individual diastereomers thereof, which includes, for example, methyl ((R)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((S)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate.

The term reverse transcriptase inhibitor includes, but is not limited to a phosphonamidate moiety, such as, tenofovir alafenamide, as well as those described in US 2008/0286230 A1. Methods for preparing stereoselective phosphoramidate or phosphonamidate containing actives are described in, for example, U.S. Pat. No. 8,816,074, as well as US 2011/0245484 A1 and US 2008/0286230 A1.

### Capsid Inhibitors

As described herein the term “capsid inhibitor” includes compounds that are capable of inhibiting the expression and/or function of a capsid protein either directly or indirectly. For example, a capsid inhibitor may include, but is not limited to, any compound that inhibits capsid assembly, induces formation of non-capsid polymers, promotes excess capsid assembly or misdirected capsid assembly, affects capsid stabilization, and/or inhibits encapsidation of RNA. Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In certain embodiments, the inhibitor inhibits the level of rcDNA and downstream products of viral life cycle by at least 5%, at least 10%, at least 20%, at least 50%, at least 75%, or at least 90%. The term capsid inhibitor includes compounds described in WO 2018/172852, which patent document is specifically incorporated by reference in its entirety.

The term capsid inhibitor also includes compounds described in International Patent Applications Publication Numbers WO2013006394, WO2014106019, and WO2014089296, including the following compounds:

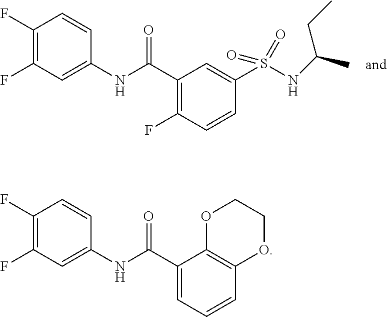 [SMILES: CC[C@@H](C)NS(=O)(=O)c1ccc(F)c(C(=O)Nc2ccc(F)c(F)c2)c1 ; O=C(Nc1ccc(F)c(F)c1)c1cccc2c1OCCO2]

The term capsid inhibitor also includes the compounds Bay-41-4109 (see International Patent Application Publication Number WO/2013/144129), AT-61 (see International Patent Application Publication Number WO/1998/33501; and King, R W, et al., Antimicrob Agents Chemother., 1998, 42, 12, 3179-3186), DVR-01 and DVR-23 (see International Patent Application Publication Number WO 2013/006394; and Campagna, M R, et al., J. of Virology, 2013, 87, 12, 6931, and pharmaceutically acceptable salts thereof:

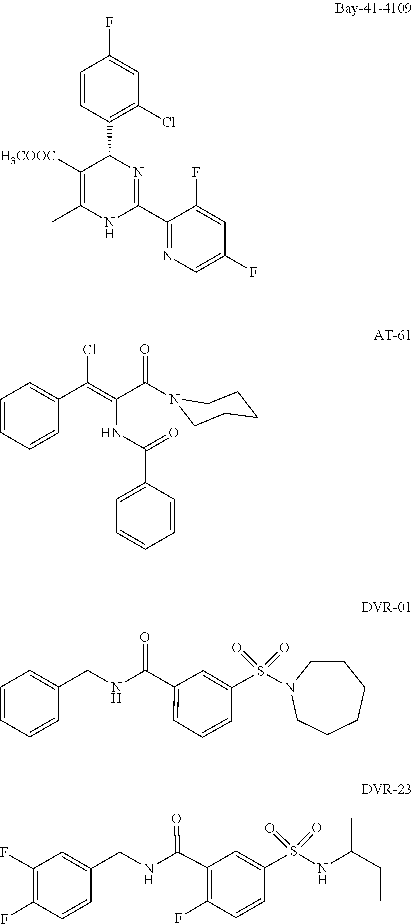

The term capsid inhibitor also includes the compound:

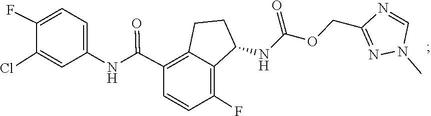 [SMILES: Cn1cnc(COC(=O)N[C@H]2CCc3c(C(=O)Nc4ccc(F)c(Cl)c4)ccc(F)c32)n1]

and pharmaceutically acceptable salts thereof (see WO 2018/172852).

In certain embodiments, a capsid inhibitor is a compound of the following formula, or a salt thereof:

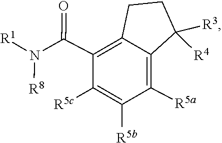

wherein the following definitions apply:

R¹ is selected from the group consisting of optionally substituted phenyl, optionally substituted benzyl, optionally substituted heteroaryl, and —(CH₂)(optionally substituted heteroaryl);

each occurrence of R² is independently selected from the group consisting of H and C₁-C₆ alkyl;

R³ is selected from the group consisting of —N(R²)C(═O)OR⁶, H, —OH, —OR⁶, —NH₂, —NHR⁶, —NR⁶R⁶, —OC(═O)OR⁶, —OC(═O)N(R²)R⁶, —NR⁷C(═O)N(R⁶)(R⁷), —N(R²)C(═O)R⁶, —NR²S(═O)₁₋₂R⁶, optionally substituted aryl, optionally substituted heteroaryl, —CH₂C(═O)OH, —CH₂C(═O)NR⁶R⁶, —N(R²)C(═O)(CH₂)₁₋₂R⁶, NR²S(═O)₂N(R⁶)(R⁷), and —NR²C(═O)C(═O)N(R⁶)(R⁷);

R⁴ is H or C₁-C₆ alkyl, or
R³ and R⁴ combine to form ═O or —C(═O)NR^{6a}—C(═O)—NR^{6a}—;

R^{5a} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl;

R^{5b} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl;

R^{5c} is independently selected from the group consisting of H, halo, C₁-C₆ alkyl, C₁-C₆ alkoxy, C₁-C₆ aminoalkyl, C₁-C₆ haloalkoxy, and C₁-C₆ haloalkyl;

each occurrence of R⁶ is independently selected from the group consisting of optionally substituted C₁-C₆ alkyl, optionally substituted C₃-C₈ cycloalkyl, optionally substituted phenyl, and optionally substituted hetereoaryl;

each occurrence of R^{6a} is independently selected from the group consisting of H, optionally substituted C₁-C₆ alkyl, optionally substituted C₃-C₈ cycloalkyl, optionally substituted phenyl, and optionally substituted hetereoaryl;

each occurrence of R⁷ is independently selected from the group consisting of H and optionally substituted C₁-C₆ alkyl;
or, if R⁶ and R⁷ are bound to the same N atom, R⁶ and R⁷ optionally combine with the N atom to which both are bound to form optionally substituted 3-7 membered heterocyclyl; and

R⁸ is selected from the group consisting of H and C₁-C₆ alkyl.

In certain embodiments, each occurrence of R⁶ or R^{6a} is independently selected from the group consisting of —(CH₂)₁₋₃-(optionally substituted heteroaryl), —(CH₂)₁₋₃-(optionally substituted heterocyclyl), and —(CH₂)₁₋₃-(optionally substituted aryl).

In certain embodiments, each occurrence of optionally substituted alkyl, optionally substituted heterocyclyl, or optionally substituted cycloalkyl is independently optionally substituted with at least one substituent selected from the group consisting of C₁-C₆ alkyl, halo, —OR^{a}, optionally substituted phenyl, optionally substituted heteroaryl, optionally substituted heterocyclyl, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

In certain embodiments, each occurrence of optionally substituted aryl or optionally substituted heteroaryl is independently optionally substituted with at least one substituent selected from the group consisting of C₁-C₆ alkyl, C₁-C₆ haloalkyl, C₁-C₆ haloalkoxy, halo, —CN, —OR^{b}, —N(R^{b})(R^{b}), —NO₂, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl.

In certain embodiments, each occurrence of optionally substituted aryl or optionally substituted heteroaryl is independently optionally substituted with at least one substituent selected from the group consisting of C₁-C₆ alkyl, C₁-C₆ haloalkyl, C₁-C₆ haloalkoxy, halo, —CN, —OR^{c}, —N(R^{c})(R^{c}), and C₁-C₆ alkoxycarbonyl, wherein each occurrence of R^{c} is independently H, C₁-C₆ alkyl, or C₃-C₈ cycloalkyl.

In certain embodiments, R¹ is selected from the group consisting of optionally substituted phenyl, optionally substituted benzyl, and —(CH₂)(optionally substituted heteroaryl), wherein the phenyl, benzyl, or heteroaryl is optionally substituted with at least one selected from the group consisting of C₁-C₆ alkyl, halo, C₁-C₃ haloalkyl, and —CN.

In certain embodiments, R¹ is selected from the group consisting of 3,4-difluorophenyl, 3,5-difluorophenyl, 2,4,5-trifluorophenyl, 3,4,5-trifluorophenyl, 3,4-dichlorophenyl, 3-chloro-4-fluorophenyl, 4-chloro-3-fluorophenyl, 4-chloro-3-methylphenyl, 3-chloro-4-methylphenyl, 4-fluoro-3-methylphenyl, 3-fluoro-4-methylphenyl, 4-chloro-3-methoxyphenyl, 3-chloro-4-methoxyphenyl, 4-fluoro-3-methoxyphenyl, 3-fluoro-4-methoxyphenyl, phenyl, 3-chlorophenyl, 4-chlorophenyl, 3-fluorophenyl, 4-fluorophenyl, 3-trifluoromethylphenyl, 4-trifluoromethylphenyl, 3-trifluoromethyl-4-fluorophenyl, 4-trifluoromethyl-3-fluorophenyl, 3-cyanophenyl, 4-cyanophenyl, 3-cyano-4-fluorophenyl, 4-cyano-3-fluorophenyl, 3-difluoromethyl-4-fluorophenyl, 4-difluoromethyl-3-fluorophenyl, benzo[d][1,3]dioxol-5-yl, 2,3-dihydrobenzo[b][1,4]dioxin-6-yl, benzyl, 3-fluorobenzyl, 4-fluorobenzyl, 3-chlorobenzyl, 4-chlorobenzyl, 2-pyridyl, 4-methyl-2-pyridyl, 5-methyl-2-pyridyl, 6-methyl-2-pyridyl, 3-pyridyl, 2-methyl-3-pyridyl, 3-methyl-3-pyridyl, 4-pyridyl, 2-methyl-4-pyridyl, and 6-methyl-4-pyridyl.

In certain embodiments, each occurrence of R² is independently selected from the group consisting of H and methyl.

In certain embodiments, R³ is selected from the group consisting of: —NH₂; —OH; —NH(pyridinyl); —NH(pyrimidinyl); —NH(pyridinyl-pyrimidinyl); —NH(pyrrolo[2,3-d]pyrimidinyl); —NHS(═O)₂(C₁-C₆ alkyl); —NHS(═O)₂(C₃-C₆ cycloalkyl); —NHS(═O)₂(CH₂)₀₋₃pyridinyl; —NHS(═O)₂(benzyl); —NHS(═O)₂(pyrazolyl); —NHS(═O)₂(morpholinyl); —NHS(═O)₂NH(C₁-C₆ alkyl); —NHS(═O)₂NH(C₃-C₆ cycloalkyl); —NHS(═O)₂NH(CH₂)₀₋₃pyridinyl; —NHS(═O)₂NH(benzyl); —NHS(═O)₂NH(pyrazolyl); —NHS(═O)₂NH(morpholinyl); —NHC(═O)(C₁-C₆ alkyl); —NHC(═O)(C₃-C₈ cycloalkyl); —NHC(═O)(C₁-C₆ haloalkyl); —NHC(═O)(pyrazolyl); —NHC(═O)(thiazolyl); —NHC(═O)(oxazolyl); —NHC(═O)(pyridinyl); —NHC(═O)(CH₂)₁₋₃(pyridinyl); —NHC(═O)(CH₂)₁₋₃(pyrazinyl); —NHC(═O)(CH₂)₁₋₃(pyrimidinyl); —NHC(═O)(CH₂)₁₋₃(quinolinyl); —NHC(═O)(CH₂)₁₋₃(isoxazolyl); —NHC(═O)(CH₂)₁₋₃(oxazolyl); —NHC(═O)(CH₂)₁₋₃(oxadiazolyl); —NHC(═O)(CH₂)₁₋₃(triazolyl); —NHC(═O)(CH₂)₁₋₃(thiazolyl); —NHC(═O)(CH₂)₁₋₃(imidazolyl); —NHC(═O)(CH₂)₁₋₃(pyrazolyl); —NHC(═O)(CH₂)₁₋₃(piperidinyl); —NHC(═O)(CH₂)₁₋₃(oxopiperidinyl); —NHC(═O)(CH₂)₁₋₃(pyrrolidinyl); —NHC(═O)(CH₂)₁₋₃(oxopyrrolidinyl); —NHC(═O)(CH₂)₁₋₃(tetrahydrofuryl); —NHC(═O)(CH₂)₁₋₃(tetrahydropyranyl); —NHC(═O)(CH₂)₁₋₃(2-oxooxazolidinyl); —NHC(═O)(CH₂)₁₋₃(morpholinyl); —NHC(═O)(CH₂)₁₋₃(thiomorpholinyl); —NHC(═O)(CH₂)₁₋₃(1-oxido-thiomorpholinyl); —NHC(═O)(CH₂)₁₋₃(1,1-dioxido-thiomorpholinyl); —NHC(═O)(CH₂)₁₋₃(oxoazetidinyl); —NHC(═O)(CH₂)₁₋₃(imidazo[1,2-a]pyridin-2-yl); —NHC(═O)(CH₂)₁₋₃C(═O)-(pyrrolidin-1-yl); —NHC(═O)O(C₁-C₆ alkyl); —NHC(═O)O(C₃-C₈ cycloalkyl); —NHC(═O)O(C₁-C₆ haloalkyl); —NHC(═O)O(CH₂)₁₋₃(pyridinyl); —NHC(═O)O(CH₂)₁₋₃(pyrazinyl); —NHC(═O)O(CH₂)₁₋₃(pyrimidinyl); —NHC(═O)O(CH₂)₁₋₃(quinolinyl); —NHC(═O)O(CH₂)₁₋₃(isoxazolyl); —NHC(═O)O(CH₂)₁₋₃(oxazolyl); —NHC(═O)O(CH₂)₁₋₃(oxadiazolyl); —NHC(═O)O(CH₂)₁₋₃(triazolyl); —NHC(═O)O(CH₂)₁₋₃(thiazolyl); —NHC(═O)O(CH₂)₁₋₃(imidazolyl); —NHC(═O)O(CH₂)₁₋₃(pyrazolyl); —NHC(═O)O(CH₂)₁₋₃(piperidinyl); —NHC(═O)O(CH₂)₁₋₃(oxopiperidinyl); —NHC(═O)O(CH₂)₁₋₃(pyrrolidinyl); —NHC(═O)O(CH₂)₁₋₃(oxopyrrolidinyl); —NHC(═O)O(CH₂)₁₋₃(tetrahydrofuryl); —NHC(═O)O(CH₂)₁₋₃(tetrahydropyranyl); —NHC(═O)O(CH₂)₁₋₃(2-oxooxazolidinyl); —NHC(═O)O(CH₂)₁₋₃(morpholinyl); —NHC(═O)O(CH₂)₁₋₃(thiomorpholinyl); —NHC(═O)O(CH₂)₁₋₃(1-oxido-thiomorpholinyl); —NHC(═O)O(CH₂)₁₋₃(1,1-dioxido-thiomorpholinyl); —NHC(═O)O(CH₂)₁₋₃(oxoazetidinyl); —NHC(═O)O(CH₂)₁₋₃(imidazo[1,2-a]pyridin-2-yl); —NHC(═O)O(CH₂)₁₋₃C(═O)-(pyrrolidin-1-yl); —NHC(═O)NH(C₁-C₆ alkyl); —NHC(═O)NH(C₃-C₈ cycloalkyl); —NHC(═O)NH(C₁-C₆ haloalkyl); —NHC(═O)NH(CH₂)₁₋₃(pyridinyl); —NHC(═O)NH(CH₂)₁₋₃(pyrazinyl); —NHC(═O)NH(CH₂)₁₋₃(pyrimidinyl); —NHC(═O)NH(CH₂)₁₋₃(quinolinyl); —NHC(═O)NH(CH₂)₁₋₃(isoxazolyl); —NHC(═O)NH(CH₂)₁₋₃(oxazolyl); —NHC(═O)NH(CH₂)₁₋₃(oxadiazolyl); —NHC(═O)NH(CH₂)₁₋₃(triazolyl); —NHC(═O)NH(CH₂)₁₋₃(thiazolyl); —NHC(═O)NH(CH₂)₁₋₃(imidazolyl); —NHC(═O)NH(CH₂)₁₋₃(pyrazolyl); —NHC(═O)NH(CH₂)₁₋₃(piperidinyl); —NHC(═O)NH(CH₂)₁₋₃(oxopiperidinyl); —NHC(═O)NH(CH₂)₁₋₃(pyrrolidinyl); —NHC(═O)NH(CH₂)₁₋₃(oxopyrrolidinyl); —NHC(═O)NH(CH₂)₁₋₃(tetrahydrofuryl); —NHC(═O)NH(CH₂)₁₋₃(tetrahydropyranyl); —NHC(═O)NH(CH₂)₁₋₃(2-oxooxazolidinyl); —NHC(═O)NH(CH₂)₁₋₃(morpholinyl); —NHC(═O)NH(CH₂)₁₋₃(thiomorpholinyl); —NHC(═O)NH(CH₂)₁₋₃(1-oxido-thiomorpholinyl); —NHC(═O)NH(CH₂)₁₋₃(1,1-dioxido-thiomorpholinyl); —NHC(═O)NH(CH₂)₁₋₃(oxoazetidinyl); —NHC(═O)NH(CH₂)₁₋₃(imidazo[1,2-a]pyridin-2-yl); —NHC(═O)NH(CH₂)₁₋₃C(═O)-(pyrrolidin-1-yl); —C(═O)NHC(═O)NH—; —C(═O)N(C₁-C₆ alkyll)C(═O)NH—; —C(═O)N((CH₂)₁₋₃pyridinyl)CONH—; wherein the alkyl, cycloalkyl, heteroaryl, heterocyclyl, aryl, or benzyl group is optionally independently substituted with at least one group selected from the group consisting of C₁-C₆ alkyl; C₁-C₆ alkoxy; C₁-C₆ haloalkyl; C₁-C₆ haloalkoxy; —NH₂, —NH(C₁-C₆ alkyl), —N(C₁-C₆ alkyl)(C₁-C₆ alkyl), halogen, —OH; —CN; phenoxy, —NHC(═O)H, —NHC(═O)C₁-C₆ alkyl, —C(═O)NH₂, —C(═O)NHC₁-C₆ alkyl, —C(═O)N(C₁-C₆ alkyl)(C₁-C₆ alkyl), tetrahydropyranyl, morpholinyl, —C(═O)CH₃, —C(═O)CH₂OH, —C(═O)NHCH₃, —C(═O)CH₂OMe, or an N-oxide thereof.

In certain embodiments, R₄ is H or CH₃.

In certain embodiments, R^{5a}, R^{5b}, and R⁵ are independently selected from the group consisting of H, F, and Cl.

In certain embodiments, one of R^{5a}, R^{5b}, and R^{5c} is F, and the two remaining are H.

In certain embodiments, the compound is selected from the group consisting of:

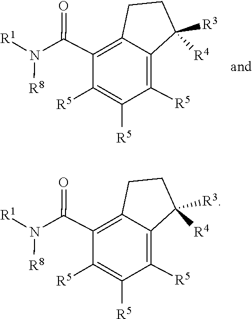

In certain embodiments, the compound is selected from the group consisting of:

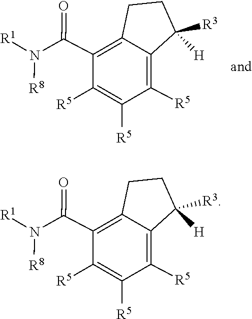

In certain embodiments, the compound is selected from the group consisting of:
O-methyl, N—(S)-(4-((3,4-difluorophenyl)carbamoyl)-2,3-dihydro-1H-inden-1-yl) carbamate;
(S)—N-(3,4-difluorophenyl)-1-(3-methylureido)-2,3-dihydro-1H-indene-4-carboxamide;
O-pyridin-2-ylmethyl, N—(S)-(4-((3,4-difluorophenyl)carbamoyl)-2,3-dihydro-1H-inden-1-yl) carbamate;
O—((R)-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-tert-butyl, N—(S)-(4-((3-chloro-4-fluorophenyl)carbamoyl)-7-fluoro-2,3-dihydro-1H-inden-1-yl) carbamate;
O-methyl, N—(S)-(7-fluoro-4-((4-fluoro-3-methylphenyl)carbamoyl)-2,3-dihydro-1H-inden-1-yl) carbamate;
(S)-7-fluoro-N-(4-fluoro-3-methylphenyl)-1-(3-methylureido)-2,3-dihydro-1H-indene-4-carboxamide;
(S)-1-amino-N-(3-chloro-4-fluorophenyl)-7-fluoro-2,3-dihydro-1H-indene-4-carboxamide;
O-2-(2-oxopyrrolidin-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N—(S)-(4-((3-chloro-4-fluorophenyl)carbamoyl)-2,3-dihydro-1H-inden-1-yl) carbamate;
O—((S)-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(3-methylureido)-2,3-dihydro-1H-indene-4-carboxamide;
O—((R)-5-oxopyrrolidin-2-yl)methyl, N—((S)-4-((3-chloro-4-fluorophenyl)carbamoyl)-2,3-dihydro-1H-inden-1-yl) carbamate;
O—((S)-5-oxopyrrolidin-2-yl)methyl, N—((S)-4-((3-chloro-4-fluorophenyl)carbamoyl)-2,3-dihydro-1H-inden-1-yl) carbamate;
O-pyridin-2-ylmethyl, N—(S)-(4-((4-fluoro-3-methylphenyl)carbamoyl)-2,3-dihydro-1H-inden-1-yl) carbamate;
O—((R)-5-oxopyrrolidin-2-yl)methyl, N—((S)-4-((4-fluoro-3-methylphenyl)carbamoyl)-2,3-dihydro-1H-inden-1-yl)carbamate;
O—((S)-5-oxopyrrolidin-2-yl)methyl, N—((S)-4-((4-fluoro-3-methylphenyl)carbamoyl)-2,3-dihydro-1H-inden-1-yl) carbamate;
O-2-oxo-2-(pyrrolidin-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N—(S)-(4-((3-chloro-4-fluorophenyl)carbamoyl)-7-fluoro-2,3-dihydro-1H-inden-1-yl) carbamate;
O—((S)-1-methyl-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-pyridin-2-ylmethyl, N—(S)-(7-fluoro-4-((4-fluoro-3-methylphenyl)carbamoyl)-2,3-dihydro-1H-inden-1-yl) carbamate;
O-imidazo[1,2-a]pyridin-2-ylmethyl, N—(S)-(4-((3-chloro-4-fluorophenyl)carbamoyl)-7-fluoro-2,3-dihydro-1H-inden-1-yl) carbamate;
O-(6-morpholinopyridin-2-yl)methyl, N—(S)-(4-((3-chloro-4-fluorophenyl)carbamoyl)-7-fluoro-2,3-dihydro-1H-inden-1-yl) carbamate;
O—((R)-1-methyl-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-(6-methoxypyridin-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(pyrimidin-2-ylamino)-2,3-dihydro-1H-indene-4-carboxamide;
O-(6-(dimethylamino) pyridin-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((5-methoxypyrimidin-2-yl)amino)-2,3-dihydro-1H-indene-4-carboxamide;
(S)—N-(3-chloro-4-fluorophenyl)-7-fluoro-1-((4-(pyridin-2-yl)pyrimidin-2-yl)amino)-2,3-dihydro-1H-indene-4-carboxamide;
tert-butyl 2-(((((S)-4-((3-chloro-4-fluorophenyl)carbamoyl)-7-fluoro-2,3-dihydro-1H-inden-1-yl)carbamoyl)oxy)methyl)-4,4-difluoropyrrolidine-1-carboxylate;
O-(4,4-difluoropyrrolidin-2-yl)methyl, N—((S)-4-((3-chloro-4-fluorophenyl)carbamoyl)-7-fluoro-2,3-dihydro-1H-inden-1-yl) carbamate;
O-(1-acetyl-4,4-difluoropyrrolidin-2-yl)methyl, N—((S)-4-((3-chloro-4-fluorophenyl)carbamoyl)-7-fluoro-2,3-dihydro-1H-inden-1-yl) carbamate;
O-(1-(2,2,2-trifluoroethyl)piperidin-4-yl)methyl, N—(S)-(4-((3-chloro-4-fluorophenyl)carbamoyl)-7-fluoro-2,3-dihydro-1H-inden-1-yl) carbamate;
(S)-2-((((4-((3-chloro-4-fluorophenyl)carbamoyl)-7-fluoro-2,3-dihydro-1H-inden-1-yl)carbamoyl)oxy)methyl)pyridine 1-oxide;
O—(S)-1-(pyridin-2-yl)ethyl, N—((S)-4-((3-chloro-4-fluorophenyl)carbamoyl)-7-fluoro-2,3-dihydro-1H-inden-1-yl) carbamate;
O—(S)-pyrrolidin-2-ylmethyl, N—((S)-4-((3-chloro-4-fluorophenyl)carbamoyl)-7-fluoro-2,3-dihydro-1H-inden-1-yl) carbamate;
O-3,3,3-trifluoropropyl, N—(S)-(4-((3-chloro-4-fluorophenyl)carbamoyl)-7-fluoro-2,3-dihydro-1H-inden-1-yl) carbamate;
O-(1-methyl-1H-pyrazol-3-yl)methyl, N—(S)-(4-((3-chloro-4-fluorophenyl)carbamoyl)-7-fluoro-2,3-dihydro-1H-inden-1-yl) carbamate;
O—(R)-5-oxopyrrolidin-3-yl, N—((S)-4-((3-chloro-4-fluorophenyl)carbamoyl)-7-fluoro-2,3-dihydro-1H-inden-1-yl) carbamate;
O-(6-methylpyridin-2-yl)methyl, N—(S)-(4-((3-chloro-4-fluorophenyl)carbamoyl)-7-fluoro-2,3-dihydro-1H-inden-1-yl) carbamate;
N—(S)-4-((3-chloro-4-fluorophenyl)carbamoyl)-7-fluoro-2,3-dihydro-1H-inden-1-yl, 0-(pyridin-2-ylmethyl) carbamate;
(S)—N-(3-chloro-4-fluorophenyl)-7-fluoro-1-(2-methoxyacetamido)-2,3-dihydro-1H-indene-4-carboxamide;
(S)—N-(3-chloro-4-fluorophenyl)-7-fluoro-1-(3-fluoropropanamido)-2,3-dihydro-1H-indene-4-carboxamide;
(S)-1-acetamido-N-(3-chloro-4-fluorophenyl)-7-fluoro-2,3-dihydro-1H-indene-4-carboxamide;
O-pyrazin-2-ylmethyl, N—(S)-(4-((3-chloro-4-fluorophenyl)carbamoyl)-7-fluoro-2,3-dihydro-1H-inden-1-yl) carbamate;
O-pyrimidin-2-ylmethyl, N—(S)-(4-((3-chloro-4-fluorophenyl)carbamoyl)-7-fluoro-2,3-dihydro-1H-inden-1-yl) carbamate;
O-(4-chloropyridin-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-hydroxy-2,3-dihydro-1H-indene-4-carboxamide;
O-isoxazol-3-ylmethyl, N—(S)-(4-((3-chloro-4-fluorophenyl)carbamoyl)-7-fluoro-2,3-dihydro-1H-inden-1-yl) carbamate;
O-2-(pyridin-2-yl)ethyl, N—(S)-(4-((3-chloro-4-fluorophenyl)carbamoyl)-7-fluoro-2,3-dihydro-1H-inden-1-yl) carbamate;
O-2,2-difluoroethyl, N—(S)-(4-((3-chloro-4-fluorophenyl)carbamoyl)-7-fluoro-2,3-dihydro-1H-inden-1-yl) carbamate;
O-pyrimidin-4-ylmethyl, N—(S)-(4-((3-chloro-4-fluorophenyl)carbamoyl)-7-fluoro-2,3-dihydro-1H-inden-1-yl) carbamate;
O-3-(2-oxopyrrolidin-1-yl)propyl, N—(S)-(4-((3-chloro-4-fluorophenyl)carbamoyl)-7-fluoro-2,3-dihydro-1H-inden-1-yl) carbamate;
O-(8-methylimidazo[1,2-a]pyridin-2-yl)methyl, N—(S)-(4-((3-chloro-4-fluorophenyl)carbamoyl)-7-fluoro-2,3-dihydro-1H-inden-1-yl) carbamate;
O-2,2,2-trifluoroethyl, N—(S)-(4-((3-chloro-4-fluorophenyl)carbamoyl)-7-fluoro-2,3-dihydro-1H-inden-1-yl) carbamate;
O—(S)-4-((3-chloro-4-fluorophenyl)carbamoyl)-7-fluoro-2,3-dihydro-1H-inden-1-yl, N-methylcarbamate;
N—(S)-4-((3-chloro-4-fluorophenyl)carbamoyl)-7-fluoro-2,3-dihydro-1H-inden-1-yl, O-(pyridin-2-ylmethyl) carbonate;
O-thiazol-5-ylmethyl, N—(S)-(4-((3-chloro-4-fluorophenyl)carbamoyl)-7-fluoro-2,3-dihydro-1H-inden-1-yl) carbamate;
O-thiazol-2-ylmethyl, N—(S)-(4-((3-chloro-4-fluorophenyl)carbamoyl)-7-fluoro-2,3-dihydro-1H-inden-1-yl) carbamate;
O-oxazol-4-ylmethyl, N—(S)-(4-((3-chloro-4-fluorophenyl)carbamoyl)-7-fluoro-2,3-dihydro-1H-inden-1-yl) carbamate;
O-oxazol-2-ylmethyl, N—(S)-(4-((3-chloro-4-fluorophenyl)carbamoyl)-7-fluoro-2,3-dihydro-1H-inden-1-yl) carbamate;
O-oxazol-5-ylmethyl, N—(S)-(4-((3-chloro-4-fluorophenyl)carbamoyl)-7-fluoro-2,3-dihydro-1H-inden-1-yl) carbamate;
O-2-(1H-imidazol-1-yl)ethyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(pyridin-2-ylamino)-2,3-dihydro-1H-indene-4-carboxamide;
(S)—N-(4-((3-chloro-4-fluorophenyl)carbamoyl)-7-fluoro-2,3-dihydro-1H-inden-1-yl)-1-methyl-1H-pyrazole-3-carboxamide;
O-2-phenoxyethyl, N—(S)-(4-((3-chloro-4-fluorophenyl)carbamoyl)-7-fluoro-2,3-dihydro-1H-inden-1-yl) carbamate;
(S)—N-(4-((3-chloro-4-fluorophenyl)carbamoyl)-7-fluoro-2,3-dihydro-1H-inden-1-yl)-1-methyl-1H-pyrazole-5-carboxamide;
(S)—N-(3-chloro-4-fluorophenyl)-7-fluoro-1-((1-methyl-1H-pyrazole)-3-sulfonamido)-2,3-dihydro-1H-indene-4-carboxamide;
O-(1-methyl-1H-1,2,4-triazol-3-yl)methyl, N—(S)-(4-((3-chloro-4-fluorophenyl)carbamoyl)-7-fluoro-2,3-dihydro-1H-inden-1-yl) carbamate;
O-(1-methyl-1H-pyrazol-5-yl)methyl, N—(S)-(4-((3-chloro-4-fluorophenyl)carbamoyl)-7-fluoro-2,3-dihydro-1H-inden-1-yl) carbamate;
(S)-2-((4-((3-chloro-4-fluorophenyl)carbamoyl)-7-fluoro-2,3-dihydro-1H-inden-1-yl)amino)pyrimidine-4-carboxamide;
O-2-(4-methylthiazol-5-yl)ethyl, N—(S)-(4-((3-chloro-4-fluorophenyl)carbamoyl)-7-fluoro-2,3-dihydro-1H-inden-1-yl) carbamate;
O-(1-isopropyl-1H-pyrazol-3-yl)methyl, N—(S)-(4-((3-chloro-4-fluorophenyl)carbamoyl)-7-fluoro-2,3-dihydro-1H-inden-1-yl) carbamate;
O-(5-methoxypyridin-2-yl)methyl, N—(S)-(4-((3-chloro-4-fluorophenyl)carbamoyl)-7-fluoro-2,3-dihydro-1H-inden-1-yl) carbamate;
O—((S)-1-(2,2,2-trifluoroethyl)pyrrolidin-2-yl)methyl, N—((S)-4-((3-chloro-4-fluorophenyl)carbamoyl)-7-fluoro-2,3-dihydro-1H-inden-1-yl) carbamate;
O-(5-fluoropyridin-2-yl)methyl, N—(S)-(4-((3-chloro-4-fluorophenyl)carbamoyl)-7-fluoro-2,3-dihydro-1H-inden-1-yl) carbamate;
O-2-(1H-pyrazol-4-yl)ethyl, N—(S)-(4-((3-chloro-4-fluorophenyl)carbamoyl)-7-fluoro-2,3-dihydro-1H-inden-1-yl) carbamate;
O-2-methoxyethyl, N—(S)-(4-((3-chloro-4-fluorophenyl)carbamoyl)-7-fluoro-2,3-dihydro-1H-inden-1-yl) carbamate;
O—((R)-tetrahydrofuran-2-yl)methyl, N—((S)-4-((3-chloro-4-fluorophenyl)carbamoyl)-7-fluoro-2,3-dihydro-1H-inden-1-yl) carbamate;
O-tetrahydro-2H-pyran-4-yl, N—(S)-(4-((3-chloro-4-fluorophenyl)carbamoyl)-7-fluoro-2,3-dihydro-1H-inden-1-yl) carbamate;
O-3-methoxypropyl, N—(S)-(4-((3-chloro-4-fluorophenyl)carbamoyl)-7-fluoro-2,3-dihydro-1H-inden-1-yl) carbamate;
(S)—N-(4-((3-chloro-4-fluorophenyl)carbamoyl)-7-fluoro-2,3-dihydro-1H-inden-1-yl)picolinamide;
(S)—N-(4-((3-chloro-4-fluorophenyl)carbamoyl)-7-fluoro-2,3-dihydro-1H-inden-1-yl)thiazole-5-carboxamide;
(S)—N-(3-chloro-4-fluorophenyl)-7-fluoro-1-(methylsulfonamido)-2,3-dihydro-1H-indene-4-carboxamide;
(S)—N-(3-chloro-4-fluorophenyl)-7-fluoro-1-(2-morpholinoacetamido)-2,3-dihydro-1H-indene-4-carboxamide;
(S)—N-(4-((3-chloro-4-fluorophenyl)carbamoyl)-7-fluoro-2,3-dihydro-1H-inden-1-yl)nicotinamide;
(S)—N-(4-((3-chloro-4-fluorophenyl)carbamoyl)-7-fluoro-2,3-dihydro-1H-inden-1-yl)isonicotinamide;
(S)-4-((3-chloro-4-fluorophenyl)carbamoyl)-7-fluoro-2,3-dihydro-1H-inden-1-yl methyl carbonate;
O-thiazol-4-ylmethyl, N—(S)-(4-((3-chloro-4-fluorophenyl)carbamoyl)-7-fluoro-2,3-dihydro-1H-inden-1-yl) carbamate;
O-3-(1H-imidazol-1-yl)propyl, N—(S)-(4-((3-chloro-4-fluorophenyl)carbamoyl)-7-fluoro-2,3-dihydro-1H-inden-1-yl) carbamate;
O-pyridin-2-ylmethyl, N—(S)-(4-((3-cyano-4-fluorophenyl)carbamoyl)-7-fluoro-2,3-dihydro-1H-inden-1-yl) carbamate;
(S)—N-(4-((3-chloro-4-fluorophenyl)carbamoyl)-7-fluoro-2,3-dihydro-1H-inden-1-yl)thiazole-2-carboxamide;
(S)—N-(3-chloro-4-fluorophenyl)-1-(cyclopropanesulfonamido)-7-fluoro-2,3-dihydro-1H-indene-4-carboxamide;
(S)—N-(4-((3-chloro-4-fluorophenyl)carbamoyl)-7-fluoro-2,3-dihydro-1H-inden-1-yl)oxazole-5-carboxamide;
O-cyclopentyl, N—(S)-(4-((3-chloro-4-fluorophenyl)carbamoyl)-7-fluoro-2,3-dihydro-1H-inden-1-yl)carbamate;
O-(2-oxo-oxazolidin-5-yl)methyl, N—((S)-4-((3-chloro-4-fluorophenyl)carbamoyl)-7-fluoro-2,3-dihydro-1H-inden-1-yl) carbamate;
O-2-(1H-pyrazol-1-yl)ethyl, N—(S)-(4-((3-chloro-4-fluorophenyl)carbamoyl)-7-fluoro-2,3-dihydro-1H-inden-1-yl) carbamate;
O-(1-methyl-1H-imidazol-2-yl)methyl, N—(S)-(4-((3-chloro-4-fluorophenyl)carbamoyl)-7-fluoro-2,3-dihydro-1H-inden-1-yl) carbamate;
O-(3-fluoropyridin-2-yl)methyl, N—(S)-(4-((3-chloro-4-fluorophenyl)carbamoyl)-7-fluoro-2,3-dihydro-1H-inden-1-yl) carbamate;
O—((R)-morpholin-3-yl)methyl, N—((S)-4-((3-chloro-4-fluorophenyl)carbamoyl)-7-fluoro-2,3-dihydro-1H-inden-1-yl) carbamate;
O-(4-methoxypyridin-2-yl)methyl, N—(S)-(4-((3-chloro-4-fluorophenyl)carbamoyl)-7-fluoro-2,3-dihydro-1H-inden-1-yl) carbamate;
O-2-hydroxyethyl, N—(S)-(4-((3-chloro-4-fluorophenyl)carbamoyl)-7-fluoro-2,3-dihydro-1H-inden-1-yl) carbamate;
O—((S)-tetrahydrofuran-2-yl)methyl, N—((S)-4-((3-chloro-4-fluorophenyl)carbamoyl)-7-fluoro-2,3-dihydro-1H-inden-1-yl)carbamate;
(S)—N-(3-chloro-4-fluorophenyl)-7-fluoro-1-(2-hydroxyacetamido)-2,3-dihydro-1H-indene-4-carboxamide;
(S)—N-(3-chloro-4-fluorophenyl)-7-fluoro-1-(3-(pyridin-3-yl)ureido)-2,3-dihydro-1H-indene-4-carboxamide;
(S)—N-(3-chloro-4-fluorophenyl)-7-fluoro-1-(3-(pyridin-4-yl)ureido)-2,3-dihydro-1H-indene-4-carboxamide;
(S)—N-(3-chloro-4-fluorophenyl)-7-fluoro-1-(thiazol-2-ylamino)-2,3-dihydro-1H-indene-4-carboxamide;
O-2-(piperidin-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N—(S)-(4-((3-(difluoromethyl)-4-fluorophenyl)carbamoyl)-7-fluoro-2,3-dihydro-1H-inden-1-yl) carbamate;
(S)—N-(3-chloro-4-fluorophenyl)-7-fluoro-1-(3-(pyridin-2-ylmethyl)ureido)-2,3-dihydro-1H-indene-4-carboxamide;
O-(6-cyanopyridin-2-yl)methyl, N—(S)-(4-((3-chloro-4-fluorophenyl)carbamoyl)-7-fluoro-2,3-dihydro-1H-inden-1-yl) carbamate;
O-quinolin-2-ylmethyl, N—(S)-(4-((3-chloro-4-fluorophenyl)carbamoyl)-7-fluoro-2,3-dihydro-1H-inden-1-yl) carbamate;
O-(5-methylpyrazin-2-yl)methyl, N—(S)-(4-((3-chloro-4-fluorophenyl)carbamoyl)-7-fluoro-2,3-dihydro-1H-inden-1-yl) carbamate;
O-2-morpholinoethyl-N—(S)-(4-((3-chloro-4-fluorophenyl)carbamoyl)-7-fluoro-2,3-dihydro-1H-inden-1-yl) carbamate;
O-[cis-4-hydroxycyclohexyl]-N—((S)-4-((3-chloro-4-fluorophenyl)carbamoyl)-7-fluoro-2,3-dihydro-1H-inden-1-yl)carbamate;
O-3-hydroxypropyl, N—(S)-(4-((3-chloro-4-fluorophenyl)carbamoyl)-7-fluoro-2,3-dihydro-1H-inden-1-yl) carbamate;
O-[trans-4-hydroxycyclohexyl]-N—((S)-4-((3-chloro-4-fluorophenyl)carbamoyl)-7-fluoro-2,3-dihydro-1H-inden-1-yl) carbamate;
O-2-acetamidoethyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-propionamido-2,3-dihydro-1H-indene-4-carboxamide;
(S)—N-(3-chloro-4-fluorophenyl)-7-fluoro-1-((4-methoxypyrimidin-2-yl)amino)-2,3-dihydro-1H-indene-4-carboxamide;
(S)—N-(3-chloro-4-fluorophenyl)-7-fluoro-1-((4-methylpyrimidin-2-yl)amino)-2,3-dihydro-1H-indene-4-carboxamide;
(S)—N-(3-chloro-4-fluorophenyl)-7-fluoro-1-((2-methoxypyrimidin-4-yl)amino)-2,3-dihydro-1H-indene-4-carboxamide;
(S)—N-(3-chloro-4-fluorophenyl)-7-fluoro-1-((5-methylpyrimidin-2-yl)amino)-2,3-dihydro-1H-indene-4-carboxamide;
(S)—N-(3-chloro-4-fluorophenyl)-7-fluoro-1-((6-methoxypyrimidin-4-yl)amino)-2,3-dihydro-1H-indene-4-carboxamide;
(S)—N-(3-chloro-4-fluorophenyl)-1-((4,6-dimethylpyrimidin-2-yl)amino)-7-fluoro-2,3-dihydro-1H-indene-4-carboxamide;
O—(S)-5-oxopyrrolidin-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((2-(pyridin-2-yl)ethyl)sulfonamido)-2,3-dihydro-1H-indene-4-carboxamide;
O-(6-(trifluoromethyl)pyridin-2-yl)methyl, N—(S)-(4-((3-chloro-4-fluorophenyl)carbamoyl)-7-fluoro-2,3-dihydro-1H-inden-1-yl) carbamate;
O-(5-(trifluoromethyl) pyridin-2-yl)methyl, N—(S)-(4-((3-chloro-4-fluorophenyl)carbamoyl)-7-fluoro-2,3-dihydro-1H-inden-1-yl) carbamate;
O—(R)-tetrahydrofuran-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(3-(1-methyl-1H-pyrazol-3-yl)propanamido)-2,3-dihydro-1H-indene-4-carboxamide;
O-(5-cyanopyridin-2-yl)methyl, N—(S)-(4-((3-chloro-4-fluorophenyl)carbamoyl)-7-fluoro-2,3-dihydro-1H-inden-1-yl) carbamate;
O-(3-methylpyrazin-2-yl)methyl, N—(S)-(4-((3-chloro-4-fluorophenyl)carbamoyl)-7-fluoro-2,3-dihydro-1H-inden-1-yl) carbamate;
O-(1-acetylpiperidin-4-yl)methyl, N—(S)-(4-((3-chloro-4-fluorophenyl)carbamoyl)-7-fluoro-2,3-dihydro-1H-inden-1-yl) carbamate;
O-(1-(2-hydroxyacetyl)piperidin-4-yl)methyl, N—(S)-(4-((3-chloro-4-fluorophenyl)carbamoyl)-7-fluoro-2,3-dihydro-1H-inden-1-yl) carbamate;
O-(1-(methylcarbamoyl)piperidin-4-yl)methyl, N—(S)-(4-((3-chloro-4-fluorophenyl)carbamoyl)-7-fluoro-2,3-dihydro-1H-inden-1-yl) carbamate;
O-(1,1-dioxidothiomorpholin-3-yl)methyl-N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-1-(cyclopropanecarboxamido)-7-fluoro-2,3-dihydro-1H-indene-4-carboxamide;
O—((S)-morpholin-3-yl)methyl, N—((S)-4-((3-chloro-4-fluorophenyl)carbamoyl)-7-fluoro-2,3-dihydro-1H-inden-1-yl) carbamate;
O—(S)-tetrahydrofuran-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((2-methoxyethyl) sulfonamido)-2,3-dihydro-1H-indene-4-carboxamide;
(S)—N-(3-chloro-4-fluorophenyl)-7-fluoro-1-(phenylsulfonamido)-2,3-dihydro-1H-indene-4-carboxamide;
(S)—N-(3-chloro-4-fluorophenyl)-7-fluoro-1-(pyridine-2-sulfonamido)-2,3-dihydro-1H-indene-4-carboxamide;
O-(1-(2-methoxyacetyl) piperidin-4-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((5-hydroxypyrimidin-2-yl)amino)-2,3-dihydro-1H-indene-4-carboxamide
O-(1H-pyrazol-3-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(3-((1-methyl-1H-pyrazol-3-yl)methyl)ureido)-2,3-dihydro-1H-indene-4-carboxamide;
O-(1H-1,2,4-triazol-3-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(pyrimidin-4-ylamino)-2,3-dihydro-1H-indene-4-carboxamide;
(S)—N-(3-chloro-4-fluorophenyl)-7-fluoro-1-((7-(4-methoxybenzyl)-7H-pyrrolo[2,3-d]pyrimidin-2-yl)amino)-2,3-dihydro-1H-indene-4-carboxamide;
O—((R)-6-oxopiperidin-2-yl)methyl, N—((S)-4-((3-chloro-4-fluorophenyl)carbamoyl)-7-fluoro-2,3-dihydro-1H-inden-1-yl) carbamate;
O—(R)-6-oxopiperidin-3-yl, N—((S)-4-((3-chloro-4-fluorophenyl)carbamoyl)-7-fluoro-2,3-dihydro-1H-inden-1-yl) carbamate;
O—(S)-6-oxopiperidin-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-1-(3-cyclopropylureido)-7-fluoro-2,3-dihydro-1H-indene-4-carboxamide;
O—((S)-6-oxopiperidin-2-yl)methyl, N—((S)-4-((3-chloro-4-fluorophenyl)carbamoyl)-7-fluoro-2,3-dihydro-1H-inden-1-yl) carbamate;
O-(4-oxoazetidin-2-yl)methyl, N—((S)-4-((3-chloro-4-fluorophenyl)carbamoyl)-7-fluoro-2,3-dihydro-1H-inden-1-yl) carbamate;
O-methyl, N-(4-((3-chloro-4-fluorophenyl)carbamoyl)-7-fluoro-1-methyl-2,3-dihydro-1H-inden-1-yl) carbamate;
N-(3-chloro-4-fluorophenyl)-7-fluoro-1-methyl-1-(3-methylureido)-2,3-dihydro-1H-indene-4-carboxamide;
(S)—N-(3-chloro-4-fluorophenyl)-1-(cyclopropanesulfonamido)-2,3-dihydro-1H-indene-4-carboxamide;
O-pyridin-2-ylmethyl, N-(4-((3-chloro-4-fluorophenyl)carbamoyl)-7-fluoro-1-methyl-2,3-dihydro-1H-inden-1-yl) carbamate;
(S)—N-(3-chloro-4-fluorophenyl)-1-((cyclopropylmethyl)sulfonamido)-7-fluoro-2,3-dihydro-1H-indene-4-carboxamide;
(S)—N-(3-chloro-4-fluorophenyl)-7-fluoro-1-((phenylmethyl)sulfonamido)-2,3-dihydro-1H-indene-4-carboxamide;
O-cyclopropyl, N—(S)-(4-((3-chloro-4-fluorophenyl)carbamoyl)-7-fluoro-2,3-dihydro-1H-inden-1-yl)carbamate;
(S)—N-(3-chloro-4-fluorophenyl)-7-fluoro-1-((N-methylsulfamoyl)amino)-2,3-dihydro-1H-indene-4-carboxamide;
(S)—N-(3-chloro-4-fluorophenyl)-7-fluoro-1-(morpholine-4-sulfonamido)-2,3-dihydro-1H-indene-4-carboxamide;
O-cyclopropyl, N—(S)-(4-((3-chloro-4-fluorophenyl)carbamoyl)-2,3-dihydro-1H-inden-1-yl) carbamate;
(S)—N-(3-chloro-4-fluorophenyl)-1-((N-methylsulfamoyl)amino)-2,3-dihydro-1H-indene-4-carboxamide;
O-(1,3,4-oxadiazol-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-1-(ethylsulfonamido)-7-fluoro-2,3-dihydro-1H-indene-4-carboxamide;
(S)—N-(3-chloro-4-fluorophenyl)-7-fluoro-1-(propylsulfonamido)-2,3-dihydro-1H-indene-4-carboxamide;
(S)—N-(4-chloro-3-fluorophenyl)-7-fluoro-1-((2-methylpropyl)sulfonamido)-2,3-dihydro-1H-indene-4-carboxamide;
(S)—N-(3-chloro-4-fluorophenyl)-7-fluoro-1-((N-isopropylsulfamoyl)amino)-2,3-dihydro-1H-indene-4-carboxamide;
(S)—N-(3-chloro-4-fluorophenyl)-7-fluoro-1-((1-methylethyl)sulfonamido)-2,3-dihydro-1H-indene-4-carboxamide;
(S)—N-(3-chloro-4-fluorophenyl)-1-(cyclopentanesulfonamido)-7-fluoro-2,3-dihydro-1H-indene-4-carboxamide;
(S)—N-(3-chloro-4-fluorophenyl)-1-(cyclohexanesulfonamido)-7-fluoro-2,3-dihydro-1H-indene-4-carboxamide;
(S)—N-(3-chloro-4-fluorophenyl)-1-((N-cyclopropylsulfamoyl)amino)-2,3-dihydro-1H-indene-4-carboxamide;
(S)—N-(3-chloro-4-fluorophenyl)-1-((N-cyclopropylsulfamoyl)amino)-7-fluoro-2,3-dihydro-1H-indene-4-carboxamide;
O-(1-(tetrahydro-2H-pyran-2-yl)-1H-1,2,4-triazol-3-yl)methyl, N—((S)-4-((3-chloro-4-fluorophenyl)carbamoyl)-7-fluoro-2,3-dihydro-1H-inden-1-yl) carbamate;
N-(3-Chloro-4-fluorophenyl)-7-fluoro-1-oxo-2,3-dihydro-1H-indene-4-carboxamide;
((1-(methyl-d₃)-1H-1,2,4-triazol-3-yl)methyl-d₂ (S)-(4-((3-chloro-4-fluorophenyl)carbamoyl)-7-fluoro-2,3-dihydro-1H-inden-1-yl)carbamate;
(S)-(3-((((4-((3-chloro-4-fluorophenyl)carbamoyl)-7-fluoro-2,3-dihydro-1H-inden-1-yl)carbamoyl)oxy)methyl)-1H-1,2,4-triazol-1-yl)methyl phosphoric acid;
(S)-(3-((((4-((3-chloro-4-fluorophenyl)carbamoyl)-7-fluoro-2,3-dihydro-1H-inden-1-yl)carbamoyl)oxy)methyl)-1H-pyrazol-1-yl)methyl phosphoric acid;
O—(S)-2-cyanoethyl, N-4-(3-chloro-4-fluorophenylcarbamoyl)-7-fluoro-2,3-dihydro-1H-inden-1-yl carbamate;
O—(S)-3-cyanopropyl, N-4-(3-chloro-4-fluorophenylcarbamoyl)-7-fluoro-2,3-dihydro-1H-inden-1-yl carbamate;
N-(3-chloro-4-fluorophenyl)-7′-fluoro-2,5-dioxo-2′,3′-dihydrospiro[imidazolidine-4,1′-indene]-4′-carboxamide;
N-(3-chloro-4-fluorophenyl)-7′-fluoro-2,5-dioxo-1-(pyridin-2-ylmethyl)-2′,3′-dihydrospiro[imidazolidine-4,1′-indene]-4′-carboxamide;
N-(3-chloro-4-fluoro-phenyl)-7′-fluoro-1-methyl-2,5-dioxo-spiro[imidazolidine-4,1′-indane]-4′-carboxamide;
(S)-1-(((S)-tert-butylsulfinyl)amino)-N-(3-chloro-4-fluorophenyl)-7-fluoro-2,3-dihydro-1H-indene-4-carboxamide;
(S)-1-(((R)-tert-butylsulfinyl)amino)-N-(3-chloro-4-fluorophenyl)-7-fluoro-2,3-dihydro-1H-indene-4-carboxamide;
   or a salt thereof.

In certain embodiments, a capsid inhibitor is a compound of the following formula, or a salt thereof:

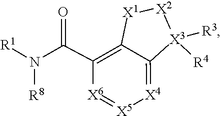

wherein the following definitions apply:

—X¹—X²— is selected from the group consisting of —CH₂CH₂—*, —CH₂CH(CH₃)—*, —CH₂C(CH₃)₂—*, —CH(CH₃)CH₂—*, —C(CH₃)₂CH₂—*, —CH₂CHF—*, —CH₂CF₂—*, —OCH₂—*, —SCH₂—*, —CH₂NR^{6a}—*, and —CH₂CH(OR^{6a})—*, wherein the single bond marked as “*” is between —X¹—X²— and X³;

X³ is C, or X³ combines with R³ and R⁴ to form —S(═O)₂—;

X⁴ is N or C(R^{5a}),

X⁵ is N or C(R^{5b}),

X⁶ is N or C(R^{5c}),
wherein 0-1 of X⁴, X⁵, and X⁶ is N;

R¹ is selected from the group consisting of optionally substituted phenyl, optionally substituted benzyl, optionally substituted heteroaryl, and —(CH₂)(optionally substituted heteroaryl);

each occurrence of R² is independently selected from the group consisting of H and C₁-C₆ alkyl;

R³ is selected from the group consisting of —N(R²)C(═O)OR⁶, H, —OH, —OR⁶, —NH₂, —NHR⁶, —NR⁶R⁶, —OC(═O)OR⁶, —OC(═O)N(R²)R⁶, —NR⁷C(═O)N(R⁶)(R⁷), —N(R²)C(═O)R⁶, —NR²S(═O)₁₋₂R⁶, optionally substituted aryl, optionally substituted heteroaryl, —CH₂C(═O)OH, —CH₂C(═O)NR⁶R⁶, —N(R²)C(═O)(CH₂)₁₋₂R⁶, NR²S(═O)₂N(R⁶)(R⁷), and —NR²C(═O)C(═O)N(R⁶)(R⁷);

R⁴ is H or C₁-C₆ alkyl,
or R³ and R⁴ combine to form ═O or —C(═O)NR^{6a}—C(═O)—NR^{6a}—;

R^{5a} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl;

R^{5b} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl;

R^{5c} is independently selected from the group consisting of H, halo, C₁-C₆ alkyl, C₁-C₆ alkoxy, C₁-C₆ aminoalkyl, C₁-C₆ haloalkoxy, and C₁-C₆ haloalkyl;

each occurrence of R⁶ is independently selected from the group consisting of optionally substituted C₁-C₆ alkyl, optionally substituted C₃-C₈ cycloalkyl, optionally substituted phenyl, and optionally substituted hetereoaryl;

each occurrence of R^{6a} is independently selected from the group consisting of H, optionally substituted C₁-C₆ alkyl, optionally substituted C₃-C₈ cycloalkyl, optionally substituted phenyl, and optionally substituted hetereoaryl;

each occurrence of R⁷ is independently selected from the group consisting of H and optionally substituted C₁-C₆ alkyl;
or, if R⁶ and R⁷ are bound to the same N atom, R⁶ and R⁷ optionally combine with the N atom to which both are bound to form an optionally substituted 3-7 membered heterocycle;

R⁸ is selected from the group consisting of H and C₁-C₆ alkyl.

In certain embodiments, a capsid inhibitor is a compound of the following formula, or a salt thereof:

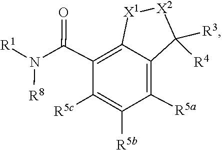

wherein the following definitions apply:

—X¹—X²— is selected from the group consisting of —CH₂CH₂—*, —CH₂CH(CH₃)—*, —CH₂C(CH₃)₂—*, —CH(CH₃)CH₂—*, —C(CH₃)₂CH₂—*, —CH₂CHF—*, —CH₂CF₂—*, —OCH₂—*, —SCH₂—*, and —CH₂CH(OR²)—*, wherein the single bond marked as “*” is between —X¹—X²— and —CR³R⁴—;

R¹ is selected from the group consisting of optionally substituted phenyl, optionally substituted benzyl, optionally substituted heteroaryl, and —(CH₂)(optionally substituted heteroaryl);

each occurrence of R² is independently selected from the group consisting of H and C₁-C₆ alkyl;

R³ is selected from the group consisting of H, —OH, —OR⁶, —NH₂, —NHR⁶, —NR⁶R⁶, —OC(═O)OR⁶, —OC(═O)N(R²)R⁶, —N(R²)C(═O)OR⁶, —NR⁷C(═O)N(R⁶)(R⁷), —N(R²)C(═O)R⁶, —NR²S(═O)₂R⁶, optionally substituted aryl, optionally substituted heteroaryl, —CH₂C(═O)OH, —CH₂C(═O)NR⁶R⁶, —N(R²)C(═O)(CH₂)₀₋₂R⁶, NR²S(═O)₂N(R⁶)(R⁷), and —NR²C(═O)C(═O)N(R⁶)(R⁷);

R⁴ is H or C₁-C₆ alkyl, or R³ and R⁴ combine to form ═O;

R^{5a} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl;

R^{5b} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl;

R^{5c} is selected from the group consisting of H, halo, C₁-C₆ alkyl, C₁-C₆ alkoxy, C₁-C₆ aminoalkyl, C₁-C₆ haloalkoxy, and C₁-C₆ haloalkyl;

each occurrence of R⁶ is independently selected from the group consisting of optionally substituted C₁-C₆ alkyl, optionally substituted C₃-C₈ cycloalkyl, optionally substituted phenyl, and optionally substituted hetereoaryl;

each occurrence of R⁷ is independently selected from the group consisting of H and optionally substituted C₁-C₆ alkyl;
or, if R⁶ and R⁷ are bound to the same N atom, R⁶ and R⁷ optionally combine with the N atom to which both are bound to form an optionally substituted 3-7 membered heterocycle;

R⁸ is selected from the group consisting of H and C₁-C₆ alkyl.

In certain embodiments, at least one of R^{5a}, R^{5b}, and R^{5c} is H.

In certain embodiments, is a compound is:

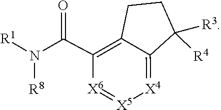

In certain embodiments, is a compound is selected from the group consisting of:

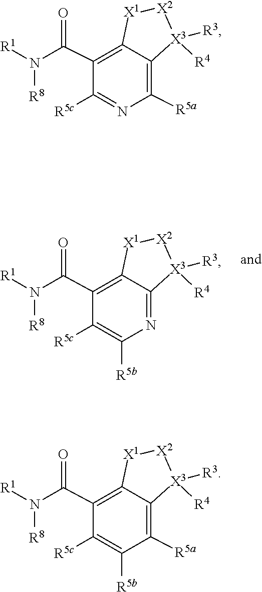

In certain embodiments, the compound is at least partially deuterated.

In certain embodiments, the compound is a prodrug.

In certain embodiments, the compound comprises a —(CRR)—O—P(═O)(OR)₂ group, or a salt thereof, which is attached to a heteroatom, wherein each occurrence of R is independently H and C₁-C₆ alkyl.

In certain embodiments, the compound is selected from the group consisting of:
O-methyl, N—(S)-(4-((3,4-difluorophenyl)carbamoyl)-2,3-dihydro-1H-inden-1-yl) carbamate;
(S)—N-(3,4-difluorophenyl)-1-(3-methylureido)-2,3-dihydro-1H-indene-4-carboxamide;
O-pyridin-2-ylmethyl, N—(S)-(4-((3,4-difluorophenyl)carbamoyl)-2,3-dihydro-1H-inden-1-yl) carbamate;
O-methyl, N-(7-((3,4-difluorophenyl)carbamoyl)-2,3-dihydrobenzofuran-3-yl) carbamate;
N-(3,4-difluorophenyl)-3-(3-methylureido)-2,3-dihydrobenzofuran-7-carboxamide;
O—((R)-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-tert-butyl, N—(S)-(4-((3-chloro-4-fluorophenyl)carbamoyl)-7-fluoro-2,3-dihydro-1H-inden-1-yl) carbamate;
O-methyl, N—(S)-(7-fluoro-4-((4-fluoro-3-methylphenyl)carbamoyl)-2,3-dihydro-1H-inden-1-yl) carbamate;
(S)-7-fluoro-N-(4-fluoro-3-methylphenyl)-1-(3-methylureido)-2,3-dihydro-1H-indene-4-carboxamide;
(S)-1-amino-N-(3-chloro-4-fluorophenyl)-7-fluoro-2,3-dihydro-1H-indene-4-carboxamide;
O-2-(2-oxopyrrolidin-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N-(7-((3,4-difluorophenyl)carbamoyl)-2,3-dihydrobenzofuran-3-yl) carbamate;
O-pyridin-2-ylmethyl, N—(S)-(4-((3-chloro-4-fluorophenyl)carbamoyl)-2,3-dihydro-1H-inden-1-yl) carbamate;
O—((S)-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(3-methylureido)-2,3-dihydro-1H-indene-4-carboxamide;
O—((R)-5-oxopyrrolidin-2-yl)methyl, N—((S)-4-((3-chloro-4-fluorophenyl)carbamoyl)-2,3-dihydro-1H-inden-1-yl) carbamate;
O—((S)-5-oxopyrrolidin-2-yl)methyl, N—((S)-4-((3-chloro-4-fluorophenyl)carbamoyl)-2,3-dihydro-1H-inden-1-yl) carbamate;
O-pyridin-2-ylmethyl, N—(S)-(4-((4-fluoro-3-methylphenyl)carbamoyl)-2,3-dihydro-1H-inden-1-yl) carbamate;
O—((R)-5-oxopyrrolidin-2-yl)methyl, N—((S)-4-((4-fluoro-3-methylphenyl)carbamoyl)-2,3-dihydro-1H-inden-1-yl)carbamate;
O—((S)-5-oxopyrrolidin-2-yl)methyl, N—((S)-4-((4-fluoro-3-methylphenyl)carbamoyl)-2,3-dihydro-1H-inden-1-yl) carbamate;
O-2-oxo-2-(pyrrolidin-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N-(7-((3,4-difluorophenyl)carbamoyl)-2,3-dihydrobenzo[b]thiophen-3-yl) carbamate;
O-pyridin-2-ylmethyl, N-(7-((3-chloro-4-fluorophenyl)carbamoyl)-2,3-dihydrobenzo[b]thiophen-3-yl) carbamate;
O-methyl, N-(7-((3-chloro-4-fluorophenyl)carbamoyl)-2,3-dihydrobenzo[b]thiophen-3-yl) carbamate;
O-methyl, N-(7-((3,4-difluorophenyl)carbamoyl)-2,3-dihydrobenzo[b]thiophen-3-yl) carbamate;
O-pyridin-2-ylmethyl, N—(S)-(4-((3-chloro-4-fluorophenyl)carbamoyl)-7-fluoro-2,3-dihydro-1H-inden-1-yl) carbamate;
O-methyl, N-(7-((3-chloro-4-fluorophenyl)carbamoyl)-2,3-dihydrobenzo[b]thiophen-3-yl) carbamate;
O—((S)-1-methyl-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-pyridin-2-ylmethyl, N—(S)-(7-fluoro-4-((4-fluoro-3-methylphenyl)carbamoyl)-2,3-dihydro-1H-inden-1-yl) carbamate;
O-imidazo[1,2-a]pyridin-2-ylmethyl, N—(S)-(4-((3-chloro-4-fluorophenyl)carbamoyl)-7-fluoro-2,3-dihydro-1H-inden-1-yl) carbamate;
O-(6-morpholinopyridin-2-yl)methyl, N—(S)-(4-((3-chloro-4-fluorophenyl)carbamoyl)-7-fluoro-2,3-dihydro-1H-inden-1-yl) carbamate;
O—((R)-1-methyl-5-oxopyrrolidin-2-yl)methyl, N—((S)-4-((3-chloro-4-fluorophenyl)carbamoyl)-7-fluoro-2,3-dihydro-1H-inden-1-yl) carbamate;
O-(6-methoxypyridin-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(pyrimidin-2-ylamino)-2,3-dihydro-1H-indene-4-carboxamide;
O-methyl, N-((1R,2R)-4-((3-chloro-4-fluorophenyl)carbamoyl)-2-hydroxy-2,3-dihydro-1H-inden-1-yl) carbamate;
N-(3-chloro-4-fluorophenyl)-2-hydroxy-1-(3-methylureido)-2,3-dihydro-1H-indene-4-carboxamide;
O-(6-(dimethylamino) pyridin-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((5-methoxypyrimidin-2-yl)amino)-2,3-dihydro-1H-indene-4-carboxamide;
(S)—N-(3-chloro-4-fluorophenyl)-7-fluoro-1-((4-(pyridin-2-yl)pyrimidin-2-yl)amino)-2,3-dihydro-1H-indene-4-carboxamide;
O-pyridin-2-ylmethyl, N-((1R,2R)-4-((3-chloro-4-fluorophenyl)carbamoyl)-2-hydroxy-2,3-dihydro-1H-inden-1-yl) carbamate;
O-methyl, N-(4-((3,4-difluorophenyl)carbamoyl)-2-hydroxy-2,3-dihydro-1H-inden-1-yl) carbamate;
N-(3,4-difluorophenyl)-2-hydroxy-1-(3-methylureido)-2,3-dihydro-1H-indene-4-carboxamide;
tert-butyl 2-(((((S)-4-((3-chloro-4-fluorophenyl)carbamoyl)-7-fluoro-2,3-dihydro-1H-inden-1-yl)carbamoyl)oxy)methyl)-4,4-difluoropyrrolidine-1-carboxylate;
O-methyl, N-(7-((3,4-difluorophenyl)carbamoyl)-4-fluoro-2,3-dihydrobenzo[b]thiophen-3-yl) carbamate;
O-(4,4-difluoropyrrolidin-2-yl)methyl, N—((S)-4-((3-chloro-4-fluorophenyl)carbamoyl)-7-fluoro-2,3-dihydro-1H-inden-1-yl) carbamate;
O-methyl, N-(7-((3-chloro-4-fluorophenyl)carbamoyl)-4-fluoro-2,3-dihydrobenzo[b]thiophen-3-yl) carbamate;
O-pyridin-2-ylmethyl, N-((1R,2R)-4-((3,4-difluorophenyl)carbamoyl)-2-hydroxy-2,3-dihydro-1H-inden-1-yl) carbamate;
O-(1-acetyl-4,4-difluoropyrrolidin-2-yl)methyl, N—((S)-4-((3-chloro-4-fluorophenyl)carbamoyl)-7-fluoro-2,3-dihydro-1H-inden-1-yl) carbamate;
O-(1-(2,2,2-trifluoroethyl)piperidin-4-yl)methyl, N—(S)-(4-((3-chloro-4-fluorophenyl)carbamoyl)-7-fluoro-2,3-dihydro-1H-inden-1-yl) carbamate;
O-pyridin-2-ylmethyl, N-(7-((3,4-difluorophenyl)carbamoyl)-4-fluoro-2,3-dihydrobenzo[b]thiophen-3-yl) carbamate;
O-pyridin-2-ylmethyl, N-(7-((3-chloro-4-fluorophenyl)carbamoyl)-4-fluoro-2,3-dihydrobenzo[b]thiophen-3-yl) carbamate;
(S)-2-((((4-((3-chloro-4-fluorophenyl)carbamoyl)-7-fluoro-2,3-dihydro-1H-inden-1-yl)carbamoyl)oxy)methyl)pyridine 1-oxide;
O—(S)-1-(pyridin-2-yl)ethyl, N—((S)-4-((3-chloro-4-fluorophenyl)carbamoyl)-7-fluoro-2,3-dihydro-1H-inden-1-yl) carbamate;
O—(S)-pyrrolidin-2-ylmethyl, N—((S)-4-((3-chloro-4-fluorophenyl)carbamoyl)-7-fluoro-2,3-dihydro-1H-inden-1-yl) carbamate;
O-3,3,3-trifluoropropyl, N—(S)-(4-((3-chloro-4-fluorophenyl)carbamoyl)-7-fluoro-2,3-dihydro-1H-inden-1-yl) carbamate;
O-(1-methyl-1H-pyrazol-3-yl)methyl, N—(S)-(4-((3-chloro-4-fluorophenyl)carbamoyl)-7-fluoro-2,3-dihydro-1H-inden-1-yl) carbamate;
O—(R)-5-oxopyrrolidin-3-yl, N—((S)-4-((3-chloro-4-fluorophenyl)carbamoyl)-7-fluoro-2,3-dihydro-1H-inden-1-yl) carbamate;
O-(6-methylpyridin-2-yl)methyl, N—(S)-(4-((3-chloro-4-fluorophenyl)carbamoyl)-7-fluoro-2,3-dihydro-1H-inden-1-yl) carbamate;
N—(S)-4-((3-chloro-4-fluorophenyl)carbamoyl)-7-fluoro-2,3-dihydro-1H-inden-1-yl, 0-(pyridin-2-ylmethyl) carbamate;
(S)—N-(3-chloro-4-fluorophenyl)-7-fluoro-1-(2-methoxyacetamido)-2,3-dihydro-1H-indene-4-carboxamide;
(S)—N-(3-chloro-4-fluorophenyl)-7-fluoro-1-(3-fluoropropanamido)-2,3-dihydro-1H-indene-4-carboxamide;
(S)-1-acetamido-N-(3-chloro-4-fluorophenyl)-7-fluoro-2,3-dihydro-1H-indene-4-carboxamide;
O-pyrazin-2-ylmethyl, N—(S)-(4-((3-chloro-4-fluorophenyl)carbamoyl)-7-fluoro-2,3-dihydro-1H-inden-1-yl) carbamate;
O-pyrimidin-2-ylmethyl, N—(S)-(4-((3-chloro-4-fluorophenyl)carbamoyl)-7-fluoro-2,3-dihydro-1H-inden-1-yl) carbamate;
O-(4-chloropyridin-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-hydroxy-2,3-dihydro-1H-indene-4-carboxamide;
O-isoxazol-3-ylmethyl, N—(S)-(4-((3-chloro-4-fluorophenyl)carbamoyl)-7-fluoro-2,3-dihydro-1H-inden-1-yl) carbamate;
O-2-(pyridin-2-yl)ethyl, N—(S)-(4-((3-chloro-4-fluorophenyl)carbamoyl)-7-fluoro-2,3-dihydro-1H-inden-1-yl) carbamate;
O-2,2-difluoroethyl, N—(S)-(4-((3-chloro-4-fluorophenyl)carbamoyl)-7-fluoro-2,3-dihydro-1H-inden-1-yl) carbamate;
O-pyrimidin-4-ylmethyl, N—(S)-(4-((3-chloro-4-fluorophenyl)carbamoyl)-7-fluoro-2,3-dihydro-1H-inden-1-yl) carbamate;
O-3-(2-oxopyrrolidin-1-yl)propyl, N—(S)-(4-((3-chloro-4-fluorophenyl)carbamoyl)-7-fluoro-2,3-dihydro-1H-inden-1-yl) carbamate;
O-(8-methylimidazo[1,2-a]pyridin-2-yl)methyl, N—(S)-(4-((3-chloro-4-fluorophenyl)carbamoyl)-7-fluoro-2,3-dihydro-1H-inden-1-yl) carbamate;
O-2,2,2-trifluoroethyl, N—(S)-(4-((3-chloro-4-fluorophenyl)carbamoyl)-7-fluoro-2,3-dihydro-1H-inden-1-yl) carbamate;
O—(S)-4-((3-chloro-4-fluorophenyl)carbamoyl)-7-fluoro-2,3-dihydro-1H-inden-1-yl, N-methylcarbamate;
N—(S)-4-((3-chloro-4-fluorophenyl)carbamoyl)-7-fluoro-2,3-dihydro-1H-inden-1-yl, 0-(pyridin-2-ylmethyl) carbonate;
O-thiazol-5-ylmethyl, N—(S)-(4-((3-chloro-4-fluorophenyl)carbamoyl)-7-fluoro-2,3-dihydro-1H-inden-1-yl) carbamate;
O-thiazol-2-ylmethyl, N—(S)-(4-((3-chloro-4-fluorophenyl)carbamoyl)-7-fluoro-2,3-dihydro-1H-inden-1-yl) carbamate;
O-oxazol-4-ylmethyl, N—(S)-(4-((3-chloro-4-fluorophenyl)carbamoyl)-7-fluoro-2,3-dihydro-1H-inden-1-yl) carbamate;
O-oxazol-2-ylmethyl, N—(S)-(4-((3-chloro-4-fluorophenyl)carbamoyl)-7-fluoro-2,3-dihydro-1H-inden-1-yl) carbamate;
O-oxazol-5-ylmethyl, N—(S)-(4-((3-chloro-4-fluorophenyl)carbamoyl)-7-fluoro-2,3-dihydro-1H-inden-1-yl) carbamate;
O-2-(1H-imidazol-1-yl)ethyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(pyridin-2-ylamino)-2,3-dihydro-1H-indene-4-carboxamide;
(S)—N-(4-((3-chloro-4-fluorophenyl)carbamoyl)-7-fluoro-2,3-dihydro-1H-inden-1-yl)-1-methyl-1H-pyrazole-3-carboxamide;
O-2-phenoxyethyl, N—(S)-(4-((3-chloro-4-fluorophenyl)carbamoyl)-7-fluoro-2,3-dihydro-1H-inden-1-yl) carbamate;
(S)—N-(4-((3-chloro-4-fluorophenyl)carbamoyl)-7-fluoro-2,3-dihydro-1H-inden-1-yl)-1-methyl-1H-pyrazole-5-carboxamide;
(S)—N-(3-chloro-4-fluorophenyl)-7-fluoro-1-((1-methyl-1H-pyrazole)-3-sulfonamido)-2,3-dihydro-1H-indene-4-carboxamide;
O-(1-methyl-1H-1,2,4-triazol-3-yl)methyl, N—(S)-(4-((3-chloro-4-fluorophenyl)carbamoyl)-7-fluoro-2,3-dihydro-1H-inden-1-yl) carbamate;
O-(1-methyl-1H-pyrazol-5-yl)methyl, N—(S)-(4-((3-chloro-4-fluorophenyl)carbamoyl)-7-fluoro-2,3-dihydro-1H-inden-1-yl) carbamate;
(S)-2-((4-((3-chloro-4-fluorophenyl)carbamoyl)-7-fluoro-2,3-dihydro-1H-inden-1-yl)amino)pyrimidine-4-carboxamide;
O-2-(4-methylthiazol-5-yl)ethyl, N—(S)-(4-((3-chloro-4-fluorophenyl)carbamoyl)-7-fluoro-2,3-dihydro-1H-inden-1-yl) carbamate;
O-(1-isopropyl-1H-pyrazol-3-yl)methyl, N—(S)-(4-((3-chloro-4-fluorophenyl)carbamoyl)-7-fluoro-2,3-dihydro-1H-inden-1-yl) carbamate;
O-(5-methoxypyridin-2-yl)methyl, N—(S)-(4-((3-chloro-4-fluorophenyl)carbamoyl)-7-fluoro-2,3-dihydro-1H-inden-1-yl) carbamate;
O—((S)-1-(2,2,2-trifluoroethyl)pyrrolidin-2-yl)methyl, N—((S)-4-((3-chloro-4-fluorophenyl)carbamoyl)-7-fluoro-2,3-dihydro-1H-inden-1-yl) carbamate;
O-(5-fluoropyridin-2-yl)methyl, N—(S)-(4-((3-chloro-4-fluorophenyl)carbamoyl)-7-fluoro-2,3-dihydro-1H-inden-1-yl) carbamate;
O-2-(1H-pyrazol-4-yl)ethyl, N—(S)-(4-((3-chloro-4-fluorophenyl)carbamoyl)-7-fluoro-2,3-dihydro-1H-inden-1-yl) carbamate;
O-2-methoxyethyl, N—(S)-(4-((3-chloro-4-fluorophenyl)carbamoyl)-7-fluoro-2,3-dihydro-1H-inden-1-yl) carbamate;
O—((R)-tetrahydrofuran-2-yl)methyl, N—((S)-4-((3-chloro-4-fluorophenyl)carbamoyl)-7-fluoro-2,3-dihydro-1H-inden-1-yl) carbamate;
O-tetrahydro-2H-pyran-4-yl, N—(S)-(4-((3-chloro-4-fluorophenyl)carbamoyl)-7-fluoro-2,3-dihydro-1H-inden-1-yl) carbamate;
O-3-methoxypropyl, N—(S)-(4-((3-chloro-4-fluorophenyl)carbamoyl)-7-fluoro-2,3-dihydro-1H-inden-1-yl) carbamate;
(S)—N-(4-((3-chloro-4-fluorophenyl)carbamoyl)-7-fluoro-2,3-dihydro-1H-inden-1-yl)picolinamide;
(S)—N-(4-((3-chloro-4-fluorophenyl)carbamoyl)-7-fluoro-2,3-dihydro-1H-inden-1-yl)thiazole-5-carboxamide;
(S)—N-(3-chloro-4-fluorophenyl)-7-fluoro-1-(methylsulfonamido)-2,3-dihydro-1H-indene-4-carboxamide;
(S)—N-(3-chloro-4-fluorophenyl)-7-fluoro-1-(2-morpholinoacetamido)-2,3-dihydro-1H-indene-4-carboxamide;
(S)—N-(4-((3-chloro-4-fluorophenyl)carbamoyl)-7-fluoro-2,3-dihydro-1H-inden-1-yl)nicotinamide;
(S)—N-(4-((3-chloro-4-fluorophenyl)carbamoyl)-7-fluoro-2,3-dihydro-1H-inden-1-yl)isonicotinamide;
O-methyl, N-(4-((3-chloro-4-fluorophenyl)carbamoyl)-7-fluoro-2,2-dimethyl-2,3-dihydro-1H-inden-1-yl)carbamate;
(S)-4-((3-chloro-4-fluorophenyl)carbamoyl)-7-fluoro-2,3-dihydro-1H-inden-1-yl methyl carbonate;
O-thiazol-4-ylmethyl, N—(S)-(4-((3-chloro-4-fluorophenyl)carbamoyl)-7-fluoro-2,3-dihydro-1H-inden-1-yl) carbamate;
O-3-(1H-imidazol-1-yl)propyl, N—(S)-(4-((3-chloro-4-fluorophenyl)carbamoyl)-7-fluoro-2,3-dihydro-1H-inden-1-yl) carbamate;
O-pyridin-2-ylmethyl, N—(S)-(4-((3-cyano-4-fluorophenyl)carbamoyl)-7-fluoro-2,3-dihydro-1H-inden-1-yl) carbamate;
(S)—N-(4-((3-chloro-4-fluorophenyl)carbamoyl)-7-fluoro-2,3-dihydro-1H-inden-1-yl)thiazole-2-carboxamide;
(S)—N-(3-chloro-4-fluorophenyl)-1-(cyclopropanesulfonamido)-7-fluoro-2,3-dihydro-1H-indene-4-carboxamide;
(S)—N-(4-((3-chloro-4-fluorophenyl)carbamoyl)-7-fluoro-2,3-dihydro-1H-inden-1-yl)oxazole-5-carboxamide;
O-methyl, N-((1R,2R)-4-((3-chloro-4-fluorophenyl)carbamoyl)-7-fluoro-2-methoxy-2,3-dihydro-1H-inden-1-yl) carbamate;
O-cyclopentyl, N—(S)-(4-((3-chloro-4-fluorophenyl)carbamoyl)-7-fluoro-2,3-dihydro-1H-inden-1-yl)carbamate;
O-(2-oxo-oxazolidin-5-yl)methyl, N—((S)-4-((3-chloro-4-fluorophenyl)carbamoyl)-7-fluoro-2,3-dihydro-1H-inden-1-yl) carbamate;
O-2-(1H-pyrazol-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N-(4-((3-chloro-4-fluorophenyl)carbamoyl)-7-fluoro-2,2-dimethyl-2,3-dihydro-1H-inden-1-yl) carbamate;
O-(1-methyl-1H-imidazol-2-yl)methyl, N—(S)-(4-((3-chloro-4-fluorophenyl)carbamoyl)-7-fluoro-2,3-dihydro-1H-inden-1-yl) carbamate;
O-(3-fluoropyridin-2-yl)methyl, N—(S)-(4-((3-chloro-4-fluorophenyl)carbamoyl)-7-fluoro-2,3-dihydro-1H-inden-1-yl) carbamate;
O—((R)-morpholin-3-yl)methyl, N—((S)-4-((3-chloro-4-fluorophenyl)carbamoyl)-7-fluoro-2,3-dihydro-1H-inden-1-yl) carbamate;
O-(4-methoxypyridin-2-yl)methyl, N—(S)-(4-((3-chloro-4-fluorophenyl)carbamoyl)-7-fluoro-2,3-dihydro-1H-inden-1-yl) carbamate;
O-2-hydroxyethyl, N—(S)-(4-((3-chloro-4-fluorophenyl)carbamoyl)-7-fluoro-2,3-dihydro-1H-inden-1-yl) carbamate;
O—((S)-tetrahydrofuran-2-yl)methyl, N—((S)-4-((3-chloro-4-fluorophenyl)carbamoyl)-7-fluoro-2,3-dihydro-1H-inden-1-yl)carbamate;
(S)—N-(3-chloro-4-fluorophenyl)-7-fluoro-1-(2-hydroxyacetamido)-2,3-dihydro-1H-indene-4-carboxamide;
(S)—N-(3-chloro-4-fluorophenyl)-7-fluoro-1-(3-(pyridin-3-yl)ureido)-2,3-dihydro-1H-indene-4-carboxamide;
(S)—N-(3-chloro-4-fluorophenyl)-7-fluoro-1-(3-(pyridin-4-yl)ureido)-2,3-dihydro-1H-indene-4-carboxamide;
(S)—N-(3-chloro-4-fluorophenyl)-7-fluoro-1-(thiazol-2-ylamino)-2,3-dihydro-1H-indene-4-carboxamide;
O-2-(piperidin-1-yl)ethyl, N—(S)-(4-((3-chloro-4-fluorophenyl)carbamoyl)-7-fluoro-2,3-dihydro-1H-inden-1-yl) carbamate;
O-pyridin-2-ylmethyl, N—(S)-(4-((3-(difluoromethyl)-4-fluorophenyl)carbamoyl)-7-fluoro-2,3-dihydro-1H-inden-1-yl) carbamate;
(S)—N-(3-chloro-4-fluorophenyl)-7-fluoro-1-(3-(pyridin-2-ylmethyl)ureido)-2,3-dihydro-1H-indene-4-carboxamide;
O-(6-cyanopyridin-2-yl)methyl, N—(S)-(4-((3-chloro-4-fluorophenyl)carbamoyl)-7-fluoro-2,3-dihydro-1H-inden-1-yl) carbamate;
O-quinolin-2-ylmethyl, N—(S)-(4-((3-chloro-4-fluorophenyl)carbamoyl)-7-fluoro-2,3-dihydro-1H-inden-1-yl) carbamate;
O-(5-methylpyrazin-2-yl)methyl, N—(S)-(4-((3-chloro-4-fluorophenyl)carbamoyl)-7-fluoro-2,3-dihydro-1H-inden-1-yl) carbamate;
O-2-morpholinoethyl-N—(S)-(4-((3-chloro-4-fluorophenyl)carbamoyl)-7-fluoro-2,3-dihydro-1H-inden-1-yl) carbamate;
O-[cis-4-hydroxycyclohexyl]-N—((S)-4-((3-chloro-4-fluorophenyl)carbamoyl)-7-fluoro-2,3-dihydro-1H-inden-1-yl)carbamate;
O-3-hydroxypropyl, N—(S)-(4-((3-chloro-4-fluorophenyl)carbamoyl)-7-fluoro-2,3-dihydro-1H-inden-1-yl) carbamate;
O-[trans-4-hydroxycyclohexyl]-N—((S)-4-((3-chloro-4-fluorophenyl)carbamoyl)-7-fluoro-2,3-dihydro-1H-inden-1-yl) carbamate;
O-2-acetamidoethyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-propionamido-2,3-dihydro-1H-indene-4-carboxamide;
(S)—N-(3-chloro-4-fluorophenyl)-7-fluoro-1-((4-methoxypyrimidin-2-yl)amino)-2,3-dihydro-1H-indene-4-carboxamide;
(S)—N-(3-chloro-4-fluorophenyl)-7-fluoro-1-((4-methylpyrimidin-2-yl)amino)-2,3-dihydro-1H-indene-4-carboxamide;
(S)—N-(3-chloro-4-fluorophenyl)-7-fluoro-1-((2-methoxypyrimidin-4-yl)amino)-2,3-dihydro-1H-indene-4-carboxamide;
(S)—N-(3-chloro-4-fluorophenyl)-7-fluoro-1-((5-methylpyrimidin-2-yl)amino)-2,3-dihydro-1H-indene-4-carboxamide;
(S)—N-(3-chloro-4-fluorophenyl)-7-fluoro-1-((6-methoxypyrimidin-4-yl)amino)-2,3-dihydro-1H-indene-4-carboxamide;
(S)—N-(3-chloro-4-fluorophenyl)-1-((4,6-dimethylpyrimidin-2-yl)amino)-7-fluoro-2,3-dihydro-1H-indene-4-carboxamide;
(1R,2R)—N-(3-chloro-4-fluorophenyl)-2-methoxy-1-(3-methylureido)-2,3-dihydro-1H-indene-4-carboxamide;
O—(S)-5-oxopyrrolidin-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((2-(pyridin-2-yl)ethyl)sulfonamido)-2,3-dihydro-1H-indene-4-carboxamide;
O-(6-(trifluoromethyl)pyridin-2-yl)methyl, N—(S)-(4-((3-chloro-4-fluorophenyl)carbamoyl)-7-fluoro-2,3-dihydro-1H-inden-1-yl) carbamate;
O-(5-(trifluoromethyl) pyridin-2-yl)methyl, N—(S)-(4-((3-chloro-4-fluorophenyl)carbamoyl)-7-fluoro-2,3-dihydro-1H-inden-1-yl) carbamate;
O—(R)-tetrahydrofuran-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(3-(1-methyl-1H-pyrazol-3-yl)propanamido)-2,3-dihydro-1H-indene-4-carboxamide;
O-(5-cyanopyridin-2-yl)methyl, N—(S)-(4-((3-chloro-4-fluorophenyl)carbamoyl)-7-fluoro-2,3-dihydro-1H-inden-1-yl) carbamate;
O-(3-methylpyrazin-2-yl)methyl, N—(S)-(4-((3-chloro-4-fluorophenyl)carbamoyl)-7-fluoro-2,3-dihydro-1H-inden-1-yl) carbamate;
O-(1-acetylpiperidin-4-yl)methyl, N—(S)-(4-((3-chloro-4-fluorophenyl)carbamoyl)-7-fluoro-2,3-dihydro-1H-inden-1-yl) carbamate;
O-(1-(2-hydroxyacetyl)piperidin-4-yl)methyl, N—(S)-(4-((3-chloro-4-fluorophenyl)carbamoyl)-7-fluoro-2,3-dihydro-1H-inden-1-yl) carbamate;
O-(1-(methylcarbamoyl)piperidin-4-yl)methyl, N—(S)-(4-((3-chloro-4-fluorophenyl)carbamoyl)-7-fluoro-2,3-dihydro-1H-inden-1-yl) carbamate;
O-(1,1-dioxidothiomorpholin-3-yl)methyl-N—((S)-4-((3-chloro-4-fluorophenyl)carbamoyl)-7-fluoro-2,3-dihydro-1H-inden-1-yl) carbamate;
O-pyridin-2-ylmethyl, N-((1R,2R)-4-((3-chloro-4-fluorophenyl)carbamoyl)-2-methoxy-2,3-dihydro-1H-inden-1-yl) carbamate;
(S)—N-(3-chloro-4-fluorophenyl)-1-(cyclopropanecarboxamido)-7-fluoro-2,3-dihydro-1H-indene-4-carboxamide;
O—((S)-morpholin-3-yl)methyl, N—((S)-4-((3-chloro-4-fluorophenyl)carbamoyl)-7-fluoro-2,3-dihydro-1H-inden-1-yl) carbamate;
O—(S)-tetrahydrofuran-3-yl, N—((S)-4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((2-methoxyethyl) sulfonamido)-2,3-dihydro-1H-indene-4-carboxamide;
(S)—N-(3-chloro-4-fluorophenyl)-7-fluoro-1-(phenylsulfonamido)-2,3-dihydro-1H-indene-4-carboxamide;
(S)—N-(3-chloro-4-fluorophenyl)-7-fluoro-1-(pyridine-2-sulfonamido)-2,3-dihydro-1H-indene-4-carboxamide;
O-(1-(2-methoxyacetyl) piperidin-4-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-((5-hydroxypyrimidin-2-yl)amino)-2,3-dihydro-1H-indene-4-carboxamide
O-methyl, N-(7-((3-chloro-4-fluorophenyl)carbamoyl)-4-fluoro-2,3-dihydrobenzofuran-3-yl) carbamate;
N-(3-chloro-4-fluorophenyl)-4-fluoro-3-(3-methylureido)-2,3-dihydrobenzofuran-7-carboxamide;
O-pyridin-2-ylmethyl, N-(7-((3-chloro-4-fluorophenyl)carbamoyl)-4-fluoro-2,3-dihydrobenzofuran-3-yl) carbamate;
O-(1H-pyrazol-3-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(3-((1-methyl-1H-pyrazol-3-yl)methyl)ureido)-2,3-dihydro-1H-indene-4-carboxamide;
O-(1H-1,2,4-triazol-3-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-7-fluoro-1-(pyrimidin-4-ylamino)-2,3-dihydro-1H-indene-4-carboxamide;
(S)—N-(3-chloro-4-fluorophenyl)-7-fluoro-1-((7-(4-methoxybenzyl)-7H-pyrrolo[2,3-d]pyrimidin-2-yl)amino)-2,3-dihydro-1H-indene-4-carboxamide;
O—((R)-6-oxopiperidin-2-yl)methyl, N—((S)-4-((3-chloro-4-fluorophenyl)carbamoyl)-7-fluoro-2,3-dihydro-1H-inden-1-yl) carbamate;
O—(R)-6-oxopiperidin-3-yl, N—((S)-4-((3-chloro-4-fluorophenyl)carbamoyl)-7-fluoro-2,3-dihydro-1H-inden-1-yl) carbamate;
O—(S)-6-oxopiperidin-3-yl, N—((S)-4-((3-chloro-4-fluorophenyl)carbamoyl)-7-fluoro-2,3-dihydro-1H-inden-1-yl) carbamate;
O-methyl, N-(4-fluoro-7-((4-fluoro-3-methylphenyl)carbamoyl)-2,3-dihydrobenzofuran-3-yl) carbamate;
4-fluoro-N-(4-fluoro-3-methylphenyl)-3-(3-methylureido)-2,3-dihydrobenzofuran-7-carboxamide;
O-pyridin-2-ylmethyl, N-(4-fluoro-7-((4-fluoro-3-methylphenyl)carbamoyl)-2,3-dihydrobenzofuran-3-yl) carbamate;
N-(3-chloro-4-fluorophenyl)-3-(cyclopropanesulfonamido)-4-fluoro-2,3-dihydrobenzofuran-7-carboxamide;
(S)—N-(3-chloro-4-fluorophenyl)-1-(3-cyclopropylureido)-7-fluoro-2,3-dihydro-1H-indene-4-carboxamide;
O-methyl, N-(4-((3-chloro-4-fluorophenyl)carbamoyl)-2,2,7-trifluoro-2,3-dihydro-1H-inden-1-yl) carbamate;
N-(3-chloro-4-fluorophenyl)-2,2,7-trifluoro-1-(3-methylureido)-2,3-dihydro-1H-indene-4-carboxamide;
O—((S)-6-oxopiperidin-2-yl)methyl, N—((S)-4-((3-chloro-4-fluorophenyl)carbamoyl)-7-fluoro-2,3-dihydro-1H-inden-1-yl) carbamate;
O-(4-oxoazetidin-2-yl)methyl, N—((S)-4-((3-chloro-4-fluorophenyl)carbamoyl)-7-fluoro-2,3-dihydro-1H-inden-1-yl) carbamate;
O-methyl, N-(4-((3-chloro-4-fluorophenyl)carbamoyl)-7-fluoro-1-methyl-2,3-dihydro-1H-inden-1-yl) carbamate;
N-(3-chloro-4-fluorophenyl)-7-fluoro-1-methyl-1-(3-methylureido)-2,3-dihydro-1H-indene-4-carboxamide;
(S)—N-(3-chloro-4-fluorophenyl)-1-(cyclopropanesulfonamido)-2,3-dihydro-1H-indene-4-carboxamide;
O-pyridin-2-ylmethyl, N-(4-((3-chloro-4-fluorophenyl)carbamoyl)-7-fluoro-1-methyl-2,3-dihydro-1H-inden-1-yl) carbamate;
O-pyridin-2-ylmethyl, N-(4-((3-chloro-4-fluorophenyl)carbamoyl)-2,2,7-trifluoro-2,3-dihydro-1H-inden-1-yl) carbamate;
(S)—N-(3-chloro-4-fluorophenyl)-1-((cyclopropylmethyl)sulfonamido)-7-fluoro-2,3-dihydro-1H-indene-4-carboxamide;
(S)—N-(3-chloro-4-fluorophenyl)-7-fluoro-1-((phenylmethyl)sulfonamido)-2,3-dihydro-1H-indene-4-carboxamide;
O-cyclopropyl, N—(S)-(4-((3-chloro-4-fluorophenyl)carbamoyl)-7-fluoro-2,3-dihydro-1H-inden-1-yl)carbamate;
(S)—N-(3-chloro-4-fluorophenyl)-7-fluoro-1-((N-methylsulfamoyl)amino)-2,3-dihydro-1H-indene-4-carboxamide;
(S)—N-(3-chloro-4-fluorophenyl)-7-fluoro-1-(morpholine-4-sulfonamido)-2,3-dihydro-1H-indene-4-carboxamide;
O-cyclopropyl, N—(S)-(4-((3-chloro-4-fluorophenyl)carbamoyl)-2,3-dihydro-1H-inden-1-yl) carbamate;
(S)—N-(3-chloro-4-fluorophenyl)-1-((N-methylsulfamoyl)amino)-2,3-dihydro-1H-indene-4-carboxamide;
O-(1,3,4-oxadiazol-2-yl)methyl, N—(S)-(4-((3-chloro-4-fluorophenyl)carbamoyl)-7-fluoro-2,3-dihydro-1H-inden-1-yl) carbamate;
(S)—N-(3-chloro-4-fluorophenyl)-1-(ethylsulfonamido)-7-fluoro-2,3-dihydro-1H-indene-4-carboxamide;
(S)—N-(3-chloro-4-fluorophenyl)-7-fluoro-1-(propylsulfonamido)-2,3-dihydro-1H-indene-4-carboxamide;
(S)—N-(4-chloro-3-fluorophenyl)-7-fluoro-1-((2-methylpropyl)sulfonamido)-2,3-dihydro-1H-indene-4-carboxamide;
N-(3-chloro-4-fluorophenyl)-7-fluoro-2-methoxy-1-(3-methylureido)-2,3-dihydro-1H-indene-4-carboxamide;
O-methyl, N-(4-((3-chloro-4-fluorophenyl)carbamoyl)-7-fluoro-2-methoxy-2,3-dihydro-1H-inden-1-yl)carbamate;
(S)—N-(3-chloro-4-fluorophenyl)-7-fluoro-1-((N-isopropylsulfamoyl)amino)-2,3-dihydro-1H-indene-4-carboxamide;
(S)—N-(3-chloro-4-fluorophenyl)-7-fluoro-1-((1-methylethyl)sulfonamido)-2,3-dihydro-1H-indene-4-carboxamide;
(S)—N-(3-chloro-4-fluorophenyl)-1-(cyclopentanesulfonamido)-7-fluoro-2,3-dihydro-1H-indene-4-carboxamide;
(S)—N-(3-chloro-4-fluorophenyl)-1-(cyclohexanesulfonamido)-7-fluoro-2,3-dihydro-1H-indene-4-carboxamide;
N-(3-chloro-4-fluorophenyl)-7-fluoro-3,3-dimethyl-1-(3-methylureido)-2,3-dihydro-1H-indene-4-carboxamide;
(S)—N-(3-chloro-4-fluorophenyl)-1-((N-cyclopropylsulfamoyl)amino)-2,3-dihydro-1H-indene-4-carboxamide;
(S)—N-(3-chloro-4-fluorophenyl)-1-((N-cyclopropylsulfamoyl)amino)-7-fluoro-2,3-dihydro-1H-indene-4-carboxamide;
O-methyl, N-(4-((3,4-difluorophenyl)carbamoyl)-7-fluoro-2-methoxy-2,3-dihydro-1H-inden-1-yl) carbamate;
N-(3,4-difluorophenyl)-7-fluoro-2-methoxy-1-(3-methylureido)-2,3-dihydro-1H-indene-4-carboxamide;
O-pyridin-2-ylmethyl, N-(4-((3,4-difluorophenyl)carbamoyl)-7-fluoro-2-methoxy-2,3-dihydro-1H-inden-1-yl)carbamate
O-pyridin-2-ylmethyl, N-(4-((3-chloro-4-fluorophenyl)carbamoyl)-7-fluoro-2-methoxy-2,3-dihydro-1H-inden-1-yl)carbamate;
O-(1-(tetrahydro-2H-pyran-2-yl)-1H-1,2,4-triazol-3-yl)methyl, N—((S)-4-((3-chloro-4-fluorophenyl)carbamoyl)-7-fluoro-2,3-dihydro-1H-inden-1-yl) carbamate;
N-(3-chloro-4-fluorophenyl)-7-fluoro-2,2-dimethyl-1-(3-methylureido)-2,3-dihydro-1H-indene-4-carboxamide;
N-(3-Chloro-4-fluorophenyl)-7-fluoro-1-oxo-2,3-dihydro-1H-indene-4-carboxamide; ((1-(methyl-d₃)-1H-1,2,4-triazol-3-yl)methyl-d₂ (S)-(4-((3-chloro-4-fluorophenyl)carbamoyl)-7-fluoro-2,3-dihydro-1H-inden-1-yl)carbamate;
(S)-(3-((((4-((3-chloro-4-fluorophenyl)carbamoyl)-7-fluoro-2,3-dihydro-1H-inden-1-yl)carbamoyl)oxy)methyl)-1H-1,2,4-triazol-1-yl)methyl phosphoric acid;
(S)-(3-((((4-((3-chloro-4-fluorophenyl)carbamoyl)-7-fluoro-2,3-dihydro-1H-inden-1-yl)carbamoyl)oxy)methyl)-1H-pyrazol-1-yl)methyl phosphoric acid;
O—(S)-2-cyanoethyl, N-4-(3-chloro-4-fluorophenylcarbamoyl)-7-fluoro-2,3-dihydro-1H-inden-1-yl carbamate;
O—(S)-3-cyanopropyl, N-4-(3-chloro-4-fluorophenylcarbamoyl)-7-fluoro-2,3-dihydro-1H-inden-1-yl carbamate;
N-(3-chloro-4-fluorophenyl)-7′-fluoro-2,5-dioxo-2′,3′-dihydrospiro[imidazolidine-4,1′-indene]-4′-carboxamide;
N-(3-chloro-4-fluorophenyl)-7′-fluoro-2,5-dioxo-1-(pyridin-2-ylmethyl)-2′,3′-dihydrospiro[imidazolidine-4,1′-indene]-4′-carboxamide;
N-(3-chloro-4-fluoro-phenyl)-7′-fluoro-1-methyl-2,5-dioxo-spiro[imidazolidine-4,1′-indane]-4′-carboxamide;
N-(3-chloro-4-fluorophenyl)-7-(3-methylureido)-6,7-dihydro-5H-cyclopenta[b]pyridine-4-carboxamide;
O-methyl, N-(4-((3-chloro-4-fluorophenyl)carbamoyl)-6,7-dihydro-5H-cyclopenta[b]pyridin-7-yl)carbamate;
O-pyridin-2-ylmethyl, N-(4-((3-chloro-4-fluorophenyl)carbamoyl)-6,7-dihydro-5H-cyclopenta[b]pyridin-7-yl) carbamate;
N-(3-chloro-4-fluorophenyl)-7-(cyclopropanesulfonamido)-6,7-dihydro-5H-cyclopenta[c]pyridine-4-carboxamide;
O-(pyridin-2-ylmethyl)-N-[(4-((3-chloro-4-fluorophenyl)carbamoyl)-6,7-dihydro-5H-cyclopenta[c]pyridin-7-yl)] carbamate;
N-(3-chloro-4-fluorophenyl)-7-fluoro-2,3-dihydrobenzo[b]thiophene-4-carboxamide 1,1-dioxide;
N-(3-chloro-4-fluorophenyl)-2,3-dihydrobenzo[b]thiophene-4-carboxamide 1,1-dioxide; 2-(tert-butyl)-N-(3-chloro-4-fluorophenyl)-2,3-dihydrobenzo[d]isothiazole-4-carboxamide 1,1-dioxide;
N-(3-chloro-4-fluorophenyl)-2,3-dihydrobenzo[d]isothiazole-4-carboxamide-1,1-dioxide;
N-(3-chloro-4-fluorophenyl)-2-(2-hydroxyethyl)-2,3-dihydrobenzo[d]isothiazole-4-carboxamide 1,1-dioxide;
N-(3-chloro-4-fluorophenyl)-2-methyl-2,3-dihydrobenzo[d]isothiazole-4-carboxamide 1,1-dioxide;
N-(3-chloro-4-fluorophenyl)-2-isopropyl-2,3-dihydrobenzo[d]isothiazole-4-carboxamide 1,1-dioxide′
N-(3-chloro-4-fluorophenyl)-2-cyclopropyl-2,3-dihydrobenzo[d]isothiazole-4-carboxamide 1,1-dioxide;
(S)-1-(((S)-tert-butylsulfinyl)amino)-N-(3-chloro-4-fluorophenyl)-7-fluoro-2,3-dihydro-1H-indene-4-carboxamide;
(S)-1-(((R)-tert-butylsulfinyl)amino)-N-(3-chloro-4-fluorophenyl)-7-fluoro-2,3-dihydro-1H-indene-4-carboxamide;
O-methyl, N-(4-((3-chloro-4-fluorophenyl)carbamoyl)-7-fluoro-3,3-dimethyl-2,3-dihydro-1H-inden-1-yl) carbamate;
   or a salt thereof.
   cccDNA Formation Inhibitors

Covalently closed circular DNA (cccDNA) is generated in the cell nucleus from viral rcDNA and serves as the transcription template for viral mRNAs. As described herein, the term “cccDNA formation inhibitor” includes compounds that are capable of inhibiting the formation and/or stability of cccDNA either directly or indirectly. For example, a cccDNA formation inhibitor may include, but is not limited to, any compound that inhibits capsid disassembly, rcDNA entry into the nucleus, and/or the conversion of rcDNA into cccDNA. For example, in certain embodiments, the inhibitor detectably inhibits the formation and/or stability of the cccDNA as measured, e.g., using an assay described herein. In certain embodiments, the inhibitor inhibits the formation and/or stability of cccDNA by at least 5%, at least 10%, at least 20%, at least 50%, at least 75%, or at least 90%.

The term cccDNA formation inhibitor includes compounds described in International Patent Application Publication Number WO2013130703, including the following compound:

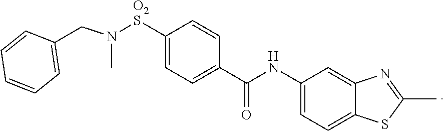 [SMILES: Cc1nc2cc(NC(=O)c3ccc(S(=O)(=O)N(C)Cc4ccccc4)cc3)ccc2s1]

The term cccDNA formation inhibitor includes, but is not limited to, those generally and specifically described in United States Patent Application Publication Number US 2015/0038515 A1. The term cccDNA formation inhibitor includes, but is not limited to, 1-(phenylsulfonyl)-N-(pyridin-4-ylmethyl)-1H-indole-2-carboxamide; 1-Benzenesulfonyl-pyrrolidine-2-carboxylic acid (pyridin-4-ylmethyl)-amide; 2-(2-chloro-N-(2-chloro-5-(trifluoromethyl)phenyl)-4-(trifluoromethyl)phenylsulfonamido)-N-(pyridin-4-ylmethyl)acetamide; 2-(4-chloro-N-(2-chloro-5-(trifluoromethyl)phenyl)phenylsulfonamido)-N-(pyridin-4-ylmethyl)acetamide; 2-(N-(2-chloro-5-(trifluoromethyl)phenyl)-4-(trifluoromethyl)phenylsulfonamido)-N-(pyridin-4-ylmethyl)acetamide; 2-(N-(2-chloro-5-(trifluoromethyl)phenyl)-4-methoxyphenylsulfonamido)-N-(pyridin-4-ylmethyl)acetamide; 2-(N-(2-chloro-5-(trifluoromethyl)phenyl)phenylsulfonamido)-N-((1-methylpiperidin-4-yl)methyl)acetamide; 2-(N-(2-chloro-5-(trifluoromethyl)phenyl)phenylsulfonamido)-N-(piperidin-4-ylmethyl)acetamide; 2-(N-(2-chloro-5-(trifluoromethyl)phenyl)phenylsulfonamido)-N-(pyridin-4-ylmethyl)propanamide; 2-(N-(2-chloro-5-(trifluoromethyl)phenyl)phenylsulfonamido)-N-(pyridin-3-ylmethyl)acetamide; 2-(N-(2-chloro-5-(trifluoromethyl)phenyl)phenylsulfonamido)-N-(pyrimidin-5-ylmethyl)acetamide; 2-(N-(2-chloro-5-(trifluoromethyl)phenyl)phenylsulfonamido)-N-(pyrimidin-4-ylmethyl)acetamide; 2-(N-(5-chloro-2-fluorophenyl)phenylsulfonamido)-N-(pyridin-4-ylmethyl)acetamide; 2-[(2-chloro-5-trifluoromethyl-phenyl)-(4-fluoro-benzenesulfonyl)-amino]-N-pyridin-4-ylmethyl-acetamide; 2-[(2-chloro-5-trifluoromethyl-phenyl)-(toluene-4-sulfonyl)-amino]-N-pyridin-4-ylmethyl-acetamide; 2-[benzenesulfonyl-(2-bromo-5-trifluoromethyl-phenyl)-amino]-N-pyridin-4-ylmethyl-acetamide; 2-[benzenesulfonyl-(2-chloro-5-trifluoromethyl-phenyl)-amino]-N-(2-methyl-benzothiazol-5-yl)-acetamide; 2-[benzenesulfonyl-(2-chloro-5-trifluoromethyl-phenyl)-amino]-N-[4-(4-methyl-piperazin-1-yl)-benzyl]-acetamide; 2-[benzenesulfonyl-(2-chloro-5-trifluoromethyl-phenyl)-amino]-N-[3-(4-methyl-piperazin-1-yl)-benzyl]-acetamide; 2-[benzenesulfonyl-(2-chloro-5-trifluoromethyl-phenyl)-amino]-N-benzyl-acetamide; 2-[benzenesulfonyl-(2-chloro-5-trifluoromethyl-phenyl)-amino]-N-pyridin-4-ylmethyl-acetamide; 2-[benzenesulfonyl-(2-chloro-5-trifluoromethyl-phenyl)-amino]-N-pyridin-4-ylmethyl-propionamide; 2-[benzenesulfonyl-(2-fluoro-5-trifluoromethyl-phenyl)-amino]-N-pyridin-4-ylmethyl-acetamide; 4 (N-(2-chloro-5-(trifluoromethyl)phenyl)phenylsulfonamido)-N-(pyridin-4-yl-methyl)butanamide; 4-((2-(N-(2-chloro-5-(trifluoromethyl)phenyl)phenylsulfonamido)-acetamido)-methyl)-1,1-dimethylpiperidin-1-ium chloride; 4-(benzyl-methyl-sulfamoyl)-N-(2-chloro-5-trifluoromethyl-phenyl)-benzamide; 4-(benzyl-methyl-sulfamoyl)-N-(2-methyl-1H-indol-5-yl)-benzamide; 4-(benzyl-methyl-sulfamoyl)-N-(2-methyl-1H-indol-5-yl)-benzamide; 4-(benzyl-methyl-sulfamoyl)-N-(2-methyl-benzothiazol-5-yl)-benzamide; 4-(benzyl-methyl-sulfamoyl)-N-(2-methyl-benzothiazol-6-yl)-benzamide; 4-(benzyl-methyl-sulfamoyl)-N-(2-methyl-benzothiazol-6-yl)-benzamide; 4-(benzyl-methyl-sulfamoyl)-N-pyridin-4-ylmethyl-benzamide; N-(2-aminoethyl)-2-(N-(2-chloro-5-(trifluoromethyl)phenyl)phenylsulfonamido)-acetamide; N-(2-chloro-5-(trifluoromethyl)phenyl)-N-(2-(3,4-dihydro-2,6-naphthyridin-2(1H)-yl)-2-oxoethyl)benzenesulfonamide; N-benzothiazol-6-yl-4-(benzyl-methyl-sulfamoyl)-benzamide; N-benzothiazol-6-yl-4-(benzyl-methyl-sulfamoyl)-benzamide; tert-butyl (2-(2-(N-(2-chloro-5-(trifluoromethyl)phenyl)phenylsulfonamido)acetamido)-ethyl)carbamate; and tert-butyl 4-((2-(N-(2-chloro-5-(trifluoromethyl)phenyl)phenylsulfonamido)-acetamido)-methyl)piperidine-1-carboxylate, and optionally, combinations thereof.

### sAg Secretion Inhibitors/RNA Destabilizers

As described herein the term “sAg secretion inhibitor” includes compounds that are capable of inhibiting, either directly or indirectly, the secretion of sAg (S, M and/or L surface antigens) bearing subviral particles and/or DNA containing viral particles from HBV-infected cells. As used herein, “sAg secretion inhibitors” are also known as “RNA destabilizers”, and these terms are used interchangeably. For example, in certain embodiments, the inhibitor detectably inhibits the secretion of sAg as measured, e.g., using assays known in the art or described herein, e.g., ELISA assay or by Western Blot. In certain embodiments, the inhibitor inhibits the secretion of sAg by at least 5%, at least 10%, at least 20%, at least 50%, at least 75%, or at least 90%. In certain embodiments, the inhibitor reduces serum levels of sAg in a patient by at least 5%, at least 10%, at least 20%, at least 50%, at least 75%, or at least 90%.

The term RNA destabilizer includes compounds described in WO 2018/085619, which patent document is specifically incorporated by reference in its entirety.

The term sAg secretion inhibitor includes compounds described in U.S. Pat. No. 8,921,381, as well as compounds described in United States Patent Application Publication Numbers 2015/0087659 and 2013/0303552. For example, the term includes the compounds PBHBV-001 and PBHBV-2-15, and pharmaceutically acceptable salts thereof:

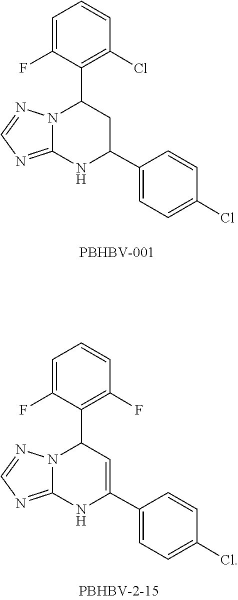 [SMILES: Fc1cccc(Cl)c1C1CC(c2ccc(Cl)cc2)Nc2ncnn21 ; Fc1cccc(F)c1C1C=C(c2ccc(Cl)cc2)Nc2ncnn21]

The term sAg secretion inhibitor/RNA destabilizer also includes the compound:

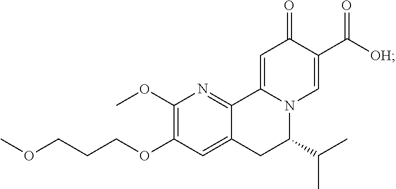 [SMILES: COCCCOc1cc2c(nc1OC)-c1cc(=O)c(C(=O)O)cn1[C@H](C(C)C)C2]

and pharmaceutically acceptable salts thereof (see WO 2018/085619).

In certain embodiments, a sAg secretion inhibitor/RNA destabilizer is a compound of the following formula, or a salt thereof:

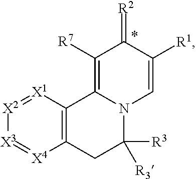

wherein the following definitions apply:

R¹ is selected from the group consisting of H; halo; —OR⁸; —C(R⁹)(R⁹)OR⁸; —C(═O)R⁸; —C(═O)OR⁸; —C(═O)NH—OR⁸; —C(═O)NHNHR⁸; —C(═O)NHNHC(═O)R⁸; —C(═O)NHS(═O)₂R⁸; —CH₂C(═O)OR⁸; —CN; —NH₂; —N(R⁸)C(═O)H; —N(R⁸)C(═O)R¹⁰; —N(R⁸)C(═O)OR¹⁰; —N(R⁸)C(═O)NHR⁸; —NR⁹S(═O)₂R¹⁰; —P(═O)(OR⁸)₂; —B(OR⁸)₂; 2,5-dioxo-pyrrolidin-1-yl; 2H-tetrazol-5-yl; 3-hydroxy-isoxazol-5-yl; 1,4-dihydro-5-oxo-5H-tetrazol-1-yl; pyridin-2-yl optionally substituted with C₁-C₆ alkyl; pyrimidin-2-yl optionally substituted with C₁-C₆ alkyl; (pyridin-2-yl)methyl; (pyrimidin-2-yl)methyl; (pyrimidin-2-yl)amino; bis-(pyrimidin-2-yl)-amino; 5-R⁸-1,3,4,-thiadiazol-2-yl; 5-thioxo-4,5-dihydro-1H-1,2,4-triazol-3-yl; 1H-1,2,4-triazol-5-yl; 1,3,4-oxadiazol-2-yl; 1,2,4-oxadiazol-5-yl, and 3-R¹⁰-1,2,4-oxadiazol-5-yl;

R² is selected from the group consisting of ═O, ═NR⁹, ═N(OR⁹), and ═N(NR⁹R⁹);
or R¹ and R² combine to form ═N—O—C(═O)— or ═N—N(R⁹)—C(═O)—, wherein the ═N group is bound to the ring carbon atom marked “*”;

X¹ is selected from the group consisting of CR^{6I} and N, X² is selected from the group consisting of CR^{6II} and N, X³ is selected from the group consisting of CR^{6III} and N, X⁴ is selected from the group consisting of CR^{6IV} and N, or either X³ and X⁴, or X¹ and X², combine to form —S—;
wherein 1-2 substituents selected from the group consisting of X¹, X², X³ and X⁴ are N; each of which, if present, is optionally alkylated with C₁-C₆ alkyl if the adjacent carbon atom in the ring is substituted with —OH;

R^{6I}, R^{6II}, R^{6III} and R^{6IV} are independently selected from the group consisting of H, halo, —CN, pyrrolidinyl, optionally substituted C₁-C₆ alkyl, optionally substituted C₁-C₆ alkenyl, optionally substituted C₃-C₈ cycloalkyl, optionally substituted heterocyclyl, —OR, C₁-C₆ haloalkoxy, —N(R)(R), —NO₂, —S(═O)₂N(R)(R), acyl, and C₁-C₆ alkoxycarbonyl,
wherein each occurrence of R is independently selected from the group consisting of H, C₁-C₆ alkyl, R′-substituted C₁-C₆ alkyl, C₁-C₆ hydroxyalkyl, optionally substituted (C₁-C₆ alkoxy)-C₁-C₆ alkyl, and optionally substituted C₃-C₈ cycloalkyl,
wherein each occurrence of R′ is independently selected from the group consisting of —NH₂, —NH(C₁-C₆ alkyl), —N(C₁-C₆ alkyl)(C₁-C₆ alkyl), —NHC(═O)O^{t}Bu, —N(C₁-C₆ alkyl)C(═O)O^{t}Bu, or a 5- or 6-membered heterocyclic group, which is optionally N-linked;
or X² is CR^{6II}, X³ is CR^{6III}, and R^{6II} and R^{6III} combine to form a divalent group selected from the group consisting of —O(CHF)O—, —O(CF₂)O—, —O(CR⁹R⁹)O—, —O(CH₂)(CH₂)O— and —O(CH₂)(CR¹¹R¹¹)(CH₂)O—;

R⁷ is selected from the group consisting of H, OH, halo, C₁-C₆ alkoxy, and optionally substituted C₁-C₆ alkyl;

R⁸ is selected from the group consisting of H, optionally substituted C₁-C₆ alkyl, and optionally substituted C₃-C₈ cycloalkyl;

each occurrence of R⁹ is independently selected from the group consisting of H and C₁-C₆ alkyl;

R¹⁰ is selected from the group consisting of optionally substituted C₁-C₆ alkyl and optionally substituted phenyl; and,

each occurrence of R¹¹ is independently selected from the group consisting of H, OH, C₁-C₆ alkyl, C₁-C₆ alkoxy, alkoxy-C₁-C₆ alkyl and alkoxy-C₁-C₆ alkoxy, wherein two R¹¹ groups bound to the same carbon atom are not simultaneously OH; or two R¹¹ groups combine with the carbon atom to which they are bound to form a moiety selected from the group consisting of C═O, C═CH₂ and oxetane-3,3-diyl.

In certain embodiments, each occurrence of alkyl or cycloalkyl is independently optionally substituted with at least one substituent selected from the group consisting of C₁-C₆ alkyl, halo, —OR″, phenyl and —N(R″)(R″), wherein each occurrence of R″ is independently H, C₁-C₆ alkyl or C₃-C₈ cycloalkyl.

In certain embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R″)(R″), —NO₂, —S(═O)₂N(R″)(R″), acyl, and C₁-C₆ alkoxycarbonyl, wherein each occurrence of R″ is independently H, C₁-C₆ alkyl or C₃-C₈ cycloalkyl.

In certain embodiments, the compound is selected from the group consisting of:

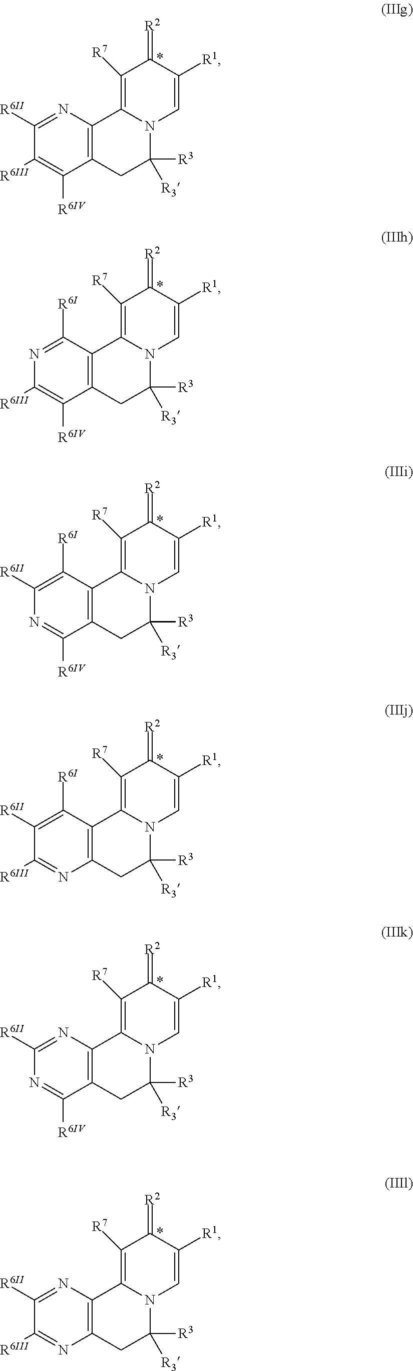

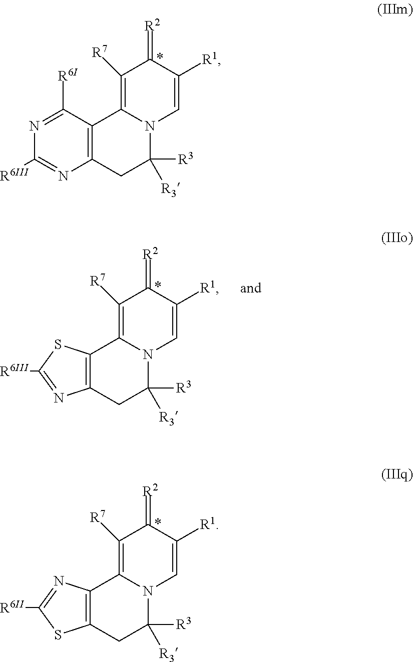

In certain embodiments, R¹ is selected from the group consisting of optionally substituted triazolyl, optionally substituted oxadiazolyl, —C(═O)OH, —C(═O)OMe, —C(═O)OEt, —C(═O)O-nPr, —C(═O)O-iPr, —C(═O)O-cyclopentyl, and —C(═O)O-cyclohexyl.

In certain embodiments, R² is selected from the group consisting of O, N(OH), N(Me), N(OMe), and N(NH₂).

In certain embodiments, R³ and R^{3′} are each independently selected from the group consisting of H, methyl, ethyl, n-propyl, isopropyl, n-butyl, isobutyl, sec-butyl, t-butyl, hydroxymethyl, 2-hydroxy-ethyl, 2-methoxy-ethyl, methoxymethyl, and 2-methyl-1-methoxy-prop-2-yl.

In certain embodiments, at least one applies: R³ is H, R^{3′} is isopropyl; R³ is H, R^{3′} is tert-butyl; R³ is methyl, R^{3′} is isopropyl; R³ is methyl, R^{3′} is tert-butyl; R³ is methyl, R^{3′} is methyl; R³ is methyl, R^{3′} is ethyl; and R³ is ethyl, R^{3′} is ethyl. In certain embodiments, R³ and R³ are not H.

In certain embodiments, R³/R^{3′} combine to form a divalent group selected from the group consisting of C₁-C₆ alkanediyl, —(CH₂)ₙO(CH₂)ₙ-, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and wherein each divalent group is optionally substituted with at least one C₁-C₆ alkyl or halo.

In certain embodiments, when present, R^{6I}, R^{6II}, R^{6III} and R^{6IV} are independently selected from the group consisting of H, F, Cl, Br, I, CN, amino, methylamino, dimethylamino, methoxyethylamino, pyrrolidinyl, methoxy, ethoxy, n-propoxy, isopropoxyl, n-butoxy, sec-butoxy, isobutoxy, t-butoxy, 2-methoxy-ethoxy, 2-hydroxy-ethoxy, 3-methoxy-prop-1-yl, 3-hydroxy-prop-1-yl, 3-methoxy-prop-1-oxy, 3-hydroxy-prop-1-oxy, 4-methoxy-but-1-yl, 4-hydroxy-but-1-yl, 4-methoxy-but-1-oxy, 4-hydroxy-but-1-oxy, 2-hydroxy-ethoxy, 3-hydroxy-prop-1-yl, 4-hydroxy-but-1-yl, 3-hydroxy-2,2-dimethyl-prop-1-oxy, cyclopropylmethoxy, 2,2,2-trifluoroethoxy, 2-(2-haloethoxy)-ethoxy, 2-(N-morpholino)-ethyl, 2-(N-morpholino)-ethoxy, 3-(N-morpholino)-prop-1-yl, 3-(N-morpholino)-prop-1-oxy, 4-(N-morpholino)-but-1-yl, 4-(N-morpholino)-but-1-oxy, 2-amino-ethyl, 2-(NHC(═O)O^{t}Bu)-ethyl, 2-amino-ethoxy, 2-(NHC(═O)O^{t}Bu)-ethoxy, 3-amino-prop-1-yl, 3-(NHC(═O)O^{t}Bu)-prop-1-yl, 3-amino-prop-1-oxy, 3-(NHC(═O)O^{t}Bu)-prop-1-oxy, 4-amino-but-1-yl, 4-(NHC(═O)O^{t}Bu)-but-1-yl, 4-amino-but-1-oxy, and 4-(NHC(═O)O^{t}Bu)-but-1-oxy.

In certain embodiments, X¹ is CH or N.

In certain embodiments, X⁴ is CH.

In certain embodiments, X² is CR^{6II}, R^{6II} is not H, X³ is CR^{6III}, and R^{6III} is not H.

In certain embodiments, X¹ is N, X² is CR^{6II}, X³ is CR^{6III}, and X⁴ is CH, and one of the following applies: R^{6II} is methoxy, R^{6III} is 3-methoxy-propoxy; R^{6II} is chloro, R^{6III} is 3-methoxy-propoxy; R^{6II} is cyclopropyl, R^{6III} is 3-methoxy-propoxy; R^{6II} is methoxy, R^{6III} is methoxy; R^{6II} is chloro, R^{6III} is methoxy; and R^{6II} is cyclopropyl, R^{6III} is methoxy.

In certain embodiments, X² is CR^{6II}, X³ is CR^{6III}, and R^{6II} and R^{6III} combine to form a divalent group selected from the group consisting of —O(CHF)O—, —O(CF₂)O—, —O(CR⁹R⁹)O—, —O(CH₂)(CH₂)O—, and —O(CH₂)(CR¹¹R¹¹)(CH₂)O.

In certain embodiments, R⁷ is selected from the group consisting of H, methyl, ethyl, and fluoro.

In certain embodiments, a sAg secretion inhibitor/RNA destabilizer is a compound of the following formula, or a salt thereof:

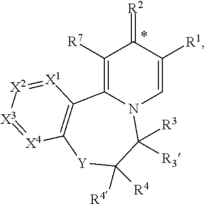

wherein the following definitions apply:

Y is selected from the group consisting of CHR⁵ and O;

each occurrence of R⁵ is independently selected from the group consisting of H, optionally substituted C₁-C₆ alkyl, and optionally substituted C₃-C₈ cycloalkyl;

R¹ is selected from the group consisting of H; halo; —OR⁸; —C(R⁹)(R⁹)OR⁸; —C(═O)R⁸; —C(═O)OR⁸; —C(═O)NH—OR⁸; —C(═O)NHNHR⁸; —C(═O)NHNHC(═O)R⁸; —C(═O)NHS(═O)₂R⁸; —CH₂C(═O)OR⁸; —CN; —NH₂; —N(R⁸)C(═O)H; —N(R⁸)C(═O)R¹⁰; —N(R⁸)C(═O)OR¹⁰; —N(R⁸)C(═O)NHR⁸; —NR⁹S(═O)₂R¹⁰; —P(═O)(OR⁸)₂; —B(OR⁸)₂; 2,5-dioxo-pyrrolidin-1-yl; 2H-tetrazol-5-yl; 3-hydroxy-isoxazol-5-yl; 1,4-dihydro-5-oxo-5H-tetrazol-1-yl; pyridin-2-yl optionally substituted with C₁-C₆ alkyl; pyrimidin-2-yl optionally substituted with C₁-C₆ alkyl; (pyridin-2-yl)methyl; (pyrimidin-2-yl)methyl; (pyrimidin-2-yl)amino; bis-(pyrimidin-2-yl)-amino; 5-R⁸-1,3,4,-thiadiazol-2-yl; 5-thioxo-4,5-dihydro-1H-1,2,4-triazol-3-yl; 1H-1,2,4-triazol-5-yl; 1,3,4-oxadiazol-2-yl; 1,2,4-oxadiazol-5-yl, and 3-R¹⁰-1,2,4-oxadiazol-5-yl;

R² is selected from the group consisting of ═O, ═NR⁹, ═N(OR⁹), and ═N(NR⁹R⁹);
or R¹ and R² combine to form ═N—O—C(═O)— or ═N—N(R⁹)—C(═O)—, wherein the ═N group is bound to the ring carbon atom marked “*”;

R³, R^{3′}, R⁴ and R^{4′} are each independently selected from the group consisting of H, alkyl-substituted oxetanyl, optionally substituted C₁-C₆ alkyl and optionally substituted C₃-C₈ cycloalkyl;
or one pair selected from the group consisting of R³/R^{3′}, R⁴/R^{4′}, and R³/R⁴ combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and each divalent group is optionally substituted with at least one C₁-C₆ alkyl or halo;

X¹ is selected from the group consisting of CR^{6I} and N, X² is selected from the group consisting of CR^{6II} and N, X³ is selected from the group consisting of CR^{6III} and N, X⁴ is selected from the group consisting of CR^{6IV} and N, or either X³ and X⁴, or X¹ and X², combine to form —S—;
wherein 0-2 substituents selected from the group consisting of X¹, X², X³ and X⁴ are N, each of which, if present, is optionally alkylated with C₁-C₆ alkyl if the adjacent carbon atom in the ring is substituted with —OH;

R^{6I}, R^{6II}, R^{6III} and R^{6IV} are independently selected from the group consisting of H, halo, —CN, pyrrolidinyl, optionally substituted C₁-C₆ alkyl, optionally substituted C₁-C₆ alkenyl, optionally substituted C₃-C₈ cycloalkyl, optionally substituted heterocyclyl, —OR, C₁-C₆ haloalkoxy, —N(R)(R), —NO₂, —S(═O)₂N(R)(R), acyl, and C₁-C₆ alkoxycarbonyl,
wherein each occurrence of R is independently selected from the group consisting of H, C₁-C₆ alkyl, R′-substituted C₁-C₆ alkyl, C₁-C₆ hydroxyalkyl, optionally substituted (C₁-C₆ alkoxy)-C₁-C₆ alkyl, and optionally substituted C₃-C₈ cycloalkyl,
wherein each occurrence of R′ is independently selected from the group consisting of —NH₂, —NH(C₁-C₆ alkyl), —N(C₁-C₆ alkyl)(C₁-C₆ alkyl), —NHC(═O)O^{t}Bu, —N(C₁-C₆ alkyl)C(═O)O^{t}Bu, or a 5- or 6-membered heterocyclic group, which is optionally N-linked;
or X² is CR^{6II}, X³ is CR^{6III}, and R^{6II} and R^{6III} combine to form a divalent group selected from the group consisting of —O(CHF)O—, —O(CF₂)O—, —O(CR⁹R⁹)O—, —O(CH₂)(CH₂)O— and —O(CH₂)(CR¹¹R¹¹)(CH₂)O—;

R⁷ is selected from the group consisting of H, OH, halo, C₁-C₆ alkoxy, and optionally substituted C₁-C₆ alkyl.

R⁸ is selected from the group consisting of H, optionally substituted C₁-C₆ alkyl, and optionally substituted C₃-C₈ cycloalkyl;

each occurrence of R⁹ is independently selected from the group consisting of H and C₁-C₆ alkyl;

R¹⁰ is selected from the group consisting of optionally substituted C₁-C₆ alkyl and optionally substituted phenyl; and,

each occurrence of R¹¹ is independently selected from the group consisting of H, OH, C₁-C₆ alkyl, C₁-C₆ alkoxy, alkoxy-C₁-C₆ alkyl and alkoxy-C₁-C₆ alkoxy, wherein two R¹¹ groups bound to the same carbon atom are not simultaneously OH; or two R¹¹ groups combine with the carbon atom to which they are bound to form a moiety selected from the group consisting of C═O, C═CH₂ and oxetane-3,3-diyl.

In certain embodiments, each occurrence of alkyl or cycloalkyl is independently optionally substituted with at least one substituent selected from the group consisting of C₁-C₆ alkyl, halo, —OR″, phenyl and —N(R″)(R″), wherein each occurrence of R″ is independently H, C₁-C₆ alkyl or C₃-C₈ cycloalkyl.

In certain embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R″)(R″), —NO₂, —S(═O)₂N(R″)(R″), acyl, and C₁-C₆ alkoxycarbonyl, wherein each occurrence of R″ is independently H, C₁-C₆ alkyl or C₃-C₈ cycloalkyl.

In certain embodiments, the compound is selected from the group consisting of:

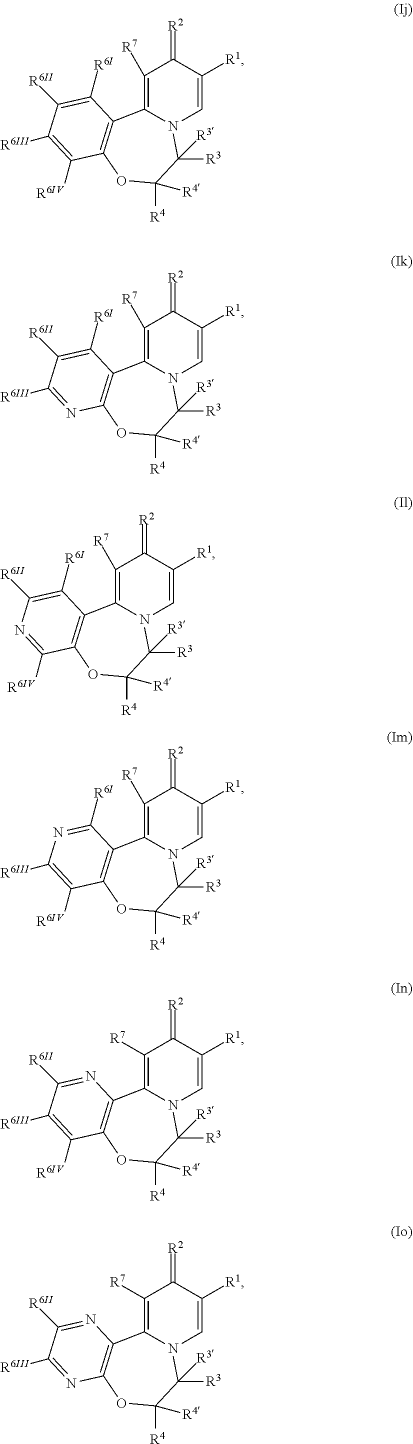

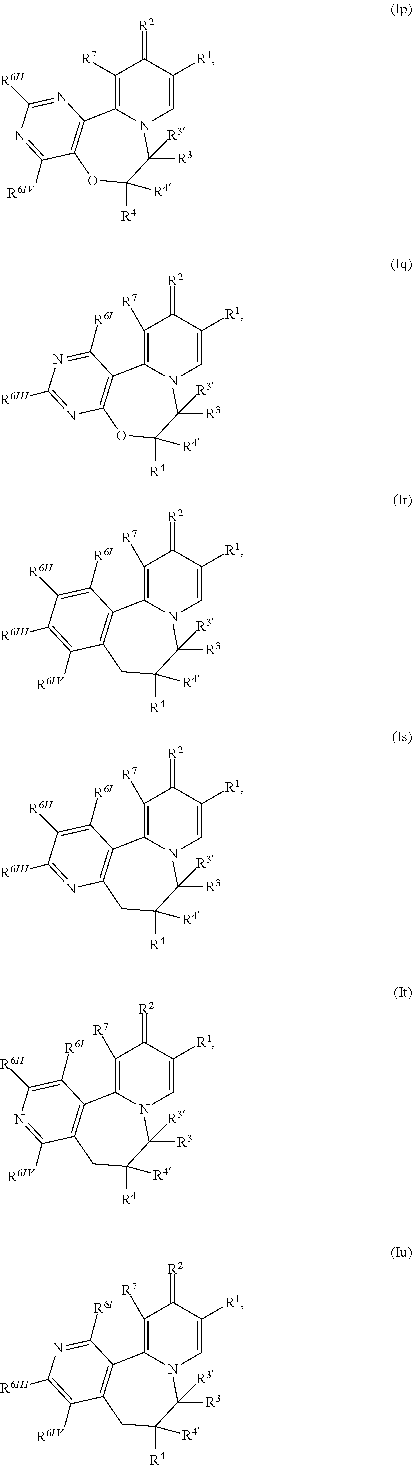

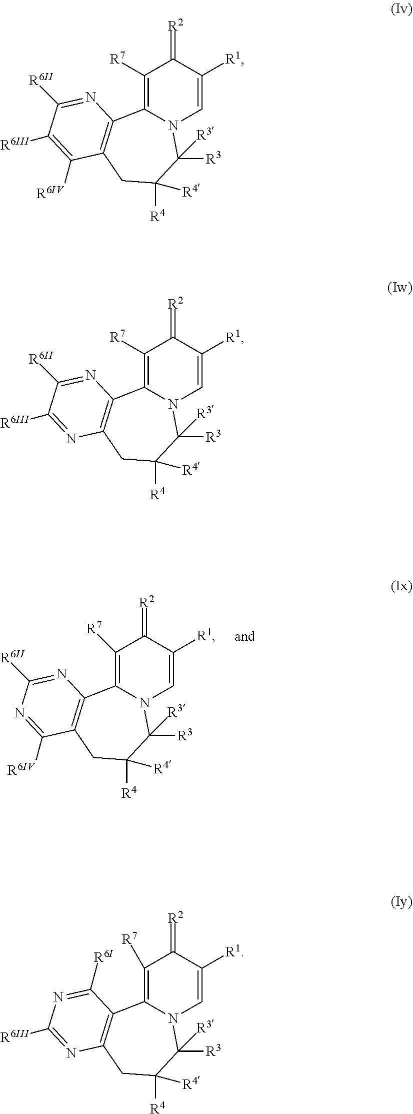

In certain embodiments, R¹ is selected from the group consisting of optionally substituted triazolyl, optionally substituted oxadiazolyl, —C(═O)OH, —C(═O)OMe, —C(═O)OEt, —C(═O)O-nPr, —C(═O)O-iPr, —C(═O)O-cyclopentyl, and —C(═O)O-cyclohexyl.

In certain embodiments, R² is selected from the group consisting of O, N(OH), N(Me), N(OMe), and N(NH₂).

In certain embodiments, R³ and R^{3′}, and R⁴ and R^{4′}, are each independently selected from the group consisting of H, methyl, ethyl, n-propyl, isopropyl, n-butyl, isobutyl, sec-butyl, t-butyl, hydroxymethyl, 2-hydroxy-ethyl, 2-methoxy-ethyl, methoxymethyl, and 2-methyl-1-methoxy-prop-2-yl.

In certain embodiments, at least one applies: R³ is H, R^{3′} is isopropyl; R³ is H, R^{3′} is tert-butyl; R³ is methyl, R^{3′} is isopropyl; R³ is methyl, R^{3′} is tert-butyl; R³ is methyl, R^{3′} is methyl; R³ is methyl, R^{3′} is ethyl; and R³ is ethyl, R^{3′} is ethyl.

In certain embodiments, R³ and R^{3′} are not H.

In certain embodiments, R⁴ and R^{4′} are H.

In certain embodiments, R³/R^{3′} combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and wherein each divalent group is optionally substituted with at least one C₁-C₆ alkyl or halo.

In certain embodiments, R^{6I}, R^{6II}, R^{6III} and R^{6IV}, when present, are independently selected from the group consisting of H, F, Cl, Br, I, CN, amino, methylamino, dimethylamino, methoxyethylamino, pyrrolidinyl, methoxy, ethoxy, n-propoxy, isopropoxyl, n-butoxy, sec-butoxy, isobutoxy, t-butoxy, 2-methoxy-ethoxy, 2-hydroxy-ethoxy, 3-methoxy-prop-1-yl, 3-hydroxy-prop-1-yl, 3-methoxy-prop-1-oxy, 3-hydroxy-prop-1-oxy, 4-methoxy-but-1-yl, 4-hydroxy-but-1-yl, 4-methoxy-but-1-oxy, 4-hydroxy-but-1-oxy, 2-hydroxy-ethoxy, 3-hydroxy-prop-1-yl, 4-hydroxy-but-1-yl, 3-hydroxy-2,2-dimethyl-prop-1-oxy, cyclopropylmethoxy, 2,2,2-trifluoroethoxy, 2-(2-haloethoxy)-ethoxy, 2-(N-morpholino)-ethyl, 2-(N-morpholino)-ethoxy, 3-(N-morpholino)-prop-1-yl, 3-(N-morpholino)-prop-1-oxy, 4-(N-morpholino)-but-1-yl, 4-(N-morpholino)-but-1-oxy, 2-amino-ethyl, 2-(NHC(═O)O^{t}Bu)-ethyl, 2-amino-ethoxy, 2-(NHC(═O)O^{t}Bu)-ethoxy, 3-amino-prop-1-yl, 3-(NHC(═O)O^{t}Bu)-prop-1-yl, 3-amino-prop-1-oxy, 3-(NHC(═O)O^{t}Bu)-prop-1-oxy, 4-amino-but-1-yl, 4-(NHC(═O)O^{t}Bu)-but-1-yl, 4-amino-but-1-oxy, and 4-(NHC(═O)O^{t}Bu)-but-1-oxy.

In certain embodiments, X¹ is CH or N.

In certain embodiments, X⁴ is CH.

In certain embodiments, X² is CR^{6II}, R^{6II} is not H, X³ is CR^{6III}, and R^{6III} is not H.

In certain embodiments, X¹ is CH, X² is CR^{6II}, X³ is CR^{6III}, and X⁴ is CH, and one of the following applies: R^{6II} is methoxy, R^{6III} is 3-methoxy-propoxy; R^{6II} is chloro, R^{6III} is 3-methoxy-propoxy; R^{6II} is isopropyl, R^{6III} is 3-methoxy-propoxy; R^{6II} is methoxy, R^{6III} is methoxy; R^{6II} is chloro, R^{6III} is methoxy; and R^{6II} is cyclopropyl, R^{6III} is methoxy.

In certain embodiments, X¹ is N, X² is CR^{6II}, X³ is CR^{6III}, and X⁴ is CH, and one of the following applies: R^{6II} is methoxy, R^{6III} is 3-methoxy-propoxy; R^{6II} is chloro, R^{6III} is 3-methoxy-propoxy; R^{6II} is cyclopropyl, R^{6III} is 3-methoxy-propoxy; R^{6II} is methoxy, R^{6III} is methoxy; R^{6II} is chloro, R^{6III} is methoxy; and R^{6II} is cyclopropyl, R^{6III} is methoxy.

In certain embodiments, X² is CR^{6II}, X³ is CR^{6III}, and R^{6II} and R^{6III} combine to form a divalent group selected from the group consisting of —O(CHF)O—, —O(CF₂)O—, —O(CR⁹R⁹)O—, —O(CH₂)(CH₂)O—, and —O(CH₂)(CR¹¹R¹¹)(CH₂)O.

In certain embodiments, R⁷ is selected from the group consisting of H, methyl, ethyl, and fluoro.

In certain embodiments, a sAg secretion inhibitor/RNA destabilizer is elected from the group consisting of compounds of formula (I), (II), and (III), or a salt thereof, wherein for the compounds of formulas (I), (II), and (III) the following definitions apply:

R¹ is selected from the group consisting of H; halo; —OR⁸; —C(R⁹)(R⁹)OR⁸; —C(═O)R⁸; —C(═O)OR⁸; —C(═O)NH—OR⁸; —C(═O)NIHNR⁸; —C(═O)NHNHC(═O)R⁸; —C(═O)NHS(═O)₂R⁸; —CH₂C(═O)OR⁸; —CN; —NH₂; —N(R⁸)C(═O)H; —N(R⁸)C(═O)R¹⁰; —N(R⁸)C(═O)OR¹⁰; —N(R⁸)C(═O)NHR⁸; —NR⁹S(═O)₂R¹⁰; —P(═O)(OR⁸)₂; —B(OR⁸)₂; 2,5-dioxo-pyrrolidin-1-yl; 2H-tetrazol-5-yl; 3-hydroxy-isoxazol-5-yl; 1,4-dihydro-5-oxo-5H-tetrazol-1-yl; pyridin-2-yl optionally substituted with C₁-C₆ alkyl; pyrimidin-2-yl optionally substituted with C₁-C₆ alkyl; (pyridin-2-yl)methyl; (pyrimidin-2-yl)methyl; (pyrimidin-2-yl)amino; bis-(pyrimidin-2-yl)-amino; 5-R⁸-1,3,4,-thiadiazol-2-yl; 5-thioxo-4,5-dihydro-1H-1,2,4-triazol-3-yl; 1H-1,2,4-triazol-5-yl; 1,3,4-oxadiazol-2-yl; 1,2,4-oxadiazol-5-yl, and 3-R¹⁰-1,2,4-oxadiazol-5-yl;

R² is selected from the group consisting of ═O, ═NR⁹, ═N(OR⁹), and ═N(NR⁹R⁹);
or R¹ and R² combine to form ═N—O—C(═O)— or ═N—N(R⁹)—C(═O)—, wherein the ═N group is bound to the ring carbon atom marked “*”;

X¹ is selected from the group consisting of CR^{6I} and N, X² is selected from the group consisting of CR^{6II} and N, X³ is selected from the group consisting of CR^{6III} and N, X⁴ is selected from the group consisting of CR^{6IV} and N, or either X³ and X⁴, or X¹ and X², combine to form —S—;
wherein 0-2 substituents selected from the group consisting of X¹, X², X³ and X⁴ are N, each of which, if present, is optionally alkylated with C₁-C₆ alkyl if the adjacent carbon atom in the ring is substituted with —OH;

R^{6I}, R^{6II}, R^{6III} and R^{6IV} are independently selected from the group consisting of H, halo, —CN, pyrrolidinyl, optionally substituted C₁-C₆ alkyl, optionally substituted C₁-C₆ alkenyl, optionally substituted C₃-C₈ cycloalkyl, optionally substituted heterocyclyl, —OR, C₁-C₆ haloalkoxy, —N(R)(R), —NO₂, —S(═O)₂N(R)(R), acyl, and C₁-C₆ alkoxycarbonyl,
wherein each occurrence of R is independently selected from the group consisting of H, C₁-C₆ alkyl, R′-substituted C₁-C₆ alkyl, C₁-C₆ hydroxyalkyl, optionally substituted (C₁-C₆ alkoxy)-C₁-C₆ alkyl, and optionally substituted C₃-C₈ cycloalkyl,
wherein each occurrence of R′ is independently selected from the group consisting of —NH₂, —NH(C₁-C₆ alkyl), —N(C₁-C₆ alkyl)(C₁-C₆ alkyl), —NHC(═O)O^{t}Bu, —N(C₁-C₆ alkyl)C(═O)O^{t}Bu, or a 5- or 6-membered heterocyclic group, which is optionally N-linked;
or X² is CR^{6II}, X³ is CR^{6III}, and R^{6II} and R^{6III} combine to form a divalent group selected from the group consisting of —O(CHF)O—, —O(CF₂)O—, —O(CR⁹R⁹)O—, —O(CH₂)(CH₂)O— and —O(CH₂)(CR¹¹R¹¹)(CH₂)O—;

R⁷ is selected from the group consisting of H, OH, halo, C₁-C₆ alkoxy, optionally substituted C₁-C₆ alkyl, and optionally substituted C₃-C₈ cycloalkyl;

R⁸ is selected from the group consisting of H, optionally substituted C₁-C₆ alkyl, and optionally substituted C₃-C₈ cycloalkyl;

each occurrence of R⁹ is independently selected from the group consisting of H and C₁-C₆ alkyl;

R¹⁰ is selected from the group consisting of optionally substituted C₁-C₆ alkyl and optionally substituted phenyl; and,

each occurrence of R¹¹ is independently selected from the group consisting of H, OH, C₁-C₆ alkyl, C₁-C₆ alkoxy, alkoxy-C₁-C₆ alkyl and alkoxy-C₁-C₆ alkoxy, wherein two R¹¹ groups bound to the same carbon atom are not simultaneously OH; or two R¹¹ groups combine with the carbon atom to which they are bound to form a moiety selected from the group consisting of C═O, C═CH₂ and oxetane-3,3-diyl;

(a) wherein the compound of formula (I) is

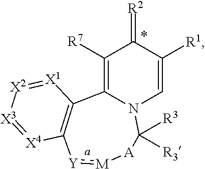

wherein in (I):

bond a is a single or double bond, wherein:
(i) if bond a is a single bond, then:
   Y is C(═O), and M is selected from the group consisting of C(R⁴)(R^{4′}) and NR⁸, or
   Y is selected from the group consisting of CHR⁵, O, S, S(═O), S(═O)₂, and NR⁵, and M is C(R⁴)(R^{4′}),
      wherein, if Y is selected from the group consisting of CHR⁵, O, and NR⁵, R⁴ and R^{4′} optionally combine with each other to form ═O; or
      Y is CH, M is C(R⁴)(R^{4′}), R^{4′} is CH₂, and Y and R^{4′} form a single bond to generate cyclopropyl;
(ii) if bond a is a double bond, then Y is selected from the group consisting of CR⁵ and N, M is C(R⁴)(R^{4′}), and R^{4′} is absent;

R³, R^{3′}, R⁴ and R^{4′} are each independently selected from the group consisting of H, alkyl-substituted oxetanyl, optionally substituted C₁-C₆ alkyl and optionally substituted C₃-C₈ cycloalkyl;
or one pair selected from the group consisting of R³/R^{3′}, R⁴/R^{4′}, and R³/R⁴ combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and each divalent group is optionally substituted with at least one C₁-C₆ alkyl or halo;

each occurrence of R⁵ is independently selected from the group consisting of H, optionally substituted C₁-C₆ alkyl, and optionally substituted C₃-C₈ cycloalkyl;

(b) wherein the compound of formula (II) is

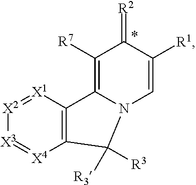

wherein in (II):

R³ and R^{3′} are each independently selected from the group consisting of H, alkyl-substituted oxetanyl, optionally substituted C₁-C₆ alkyl, and optionally substituted C₃-C₈ cycloalkyl;
or R³ and R^{3′} combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and each divalent group is optionally substituted with at least one C₁-C₆ alkyl or halo;
   (c) a compound of formula (III) is:

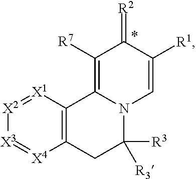

wherein in (III):

R³ and R^{3′} are each independently selected from the group consisting of H, alkyl-substituted oxetanyl, optionally substituted C₁-C₆ alkyl, and optionally substituted C₃-C₈ cycloalkyl;
or R³ and R^{3′} combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and each divalent group is optionally substituted with at least one C₁-C₆ alkyl or halo;

and

the compound of formula (III) is selected from the group consisting of:

a compound of formula (IIIa)

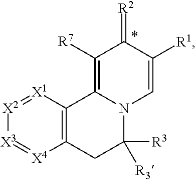

wherein 1-2 substituents selected from the group consisting of X¹, X², X³ and X⁴ are N;
a compound of formula (IIIb)

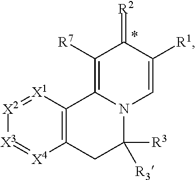

wherein at least one applies: R¹ is not —C(═O)OR⁸, R² is not ═O;
a compound of formula (IIIc)

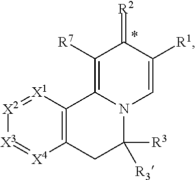

wherein X³ and X⁴, or X¹ and X², combine to form —S—;
a compound of formula (IIId)

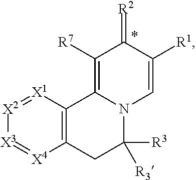

wherein X² is CR^{6II}, X³ is CR^{6III}, and R^{6II} and R^{6III} combine to form a divalent group selected from the group consisting of —O(CHF)O—, —O(CF₂)O—, —O(CR⁹R⁹)O—, —O(CH₂)(CH₂)O— and —O(CH₂)(CR¹¹R¹¹)(CH₂)O—; and
a compound of formula (IIIe)

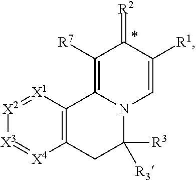

wherein R³ and R^{3′} are each independently selected from the group consisting of H, alkyl-substituted oxetanyl, optionally substituted C₁-C₆ alkyl, and optionally substituted C₃-C₈ cycloalkyl, or R³ and R^{3′} combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2, and each divalent group is optionally substituted with at least one C₁-C₆ alkyl or halo.

In certain embodiments, the compound of formula (I) is a compound of formula (Ia):

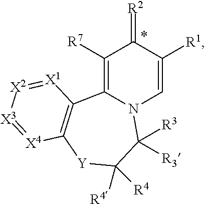

wherein in (Ia):

Y is selected from the group consisting of CHR⁵ and O; and

R³, R^{3′}, R⁴ and R^{4′} are each independently selected from the group consisting of H, alkyl-substituted oxetanyl, optionally substituted C₁-C₆ alkyl and optionally substituted C₃-C₈ cycloalkyl;
or one pair selected from the group consisting of R³/R^{3′}, R⁴/R^{4′}, and R³/R⁴ combine to form a divalent group selected from the group consisting of C₁-C₆ alkanediyl, —(CH₂)ₙO(CH₂)ₙ—, —(CH₂)ₙNR⁹(CH₂)ₙ—, —(CH₂)ₙS(CH₂)ₙ—, —(CH₂)ₙS(═O)(CH₂)ₙ—, and —(CH₂)ₙS(═O)₂(CH₂)ₙ—, wherein each occurrence of n is independently selected from the group consisting of 1 and 2 and each divalent group is optionally substituted with at least one C₁-C₆ alkyl or halo.

In certain embodiments, the compound of formula (I) is selected from the group consisting of:

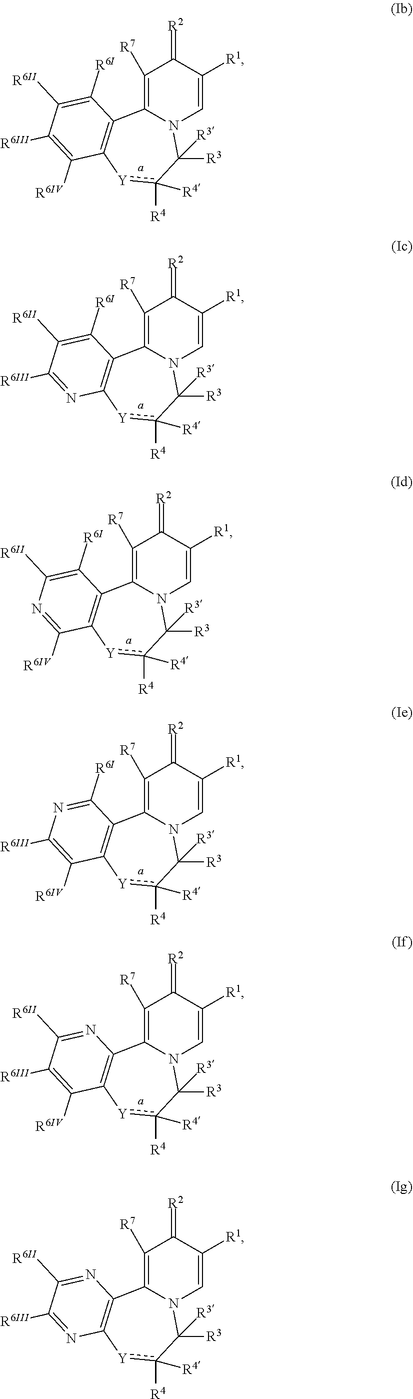

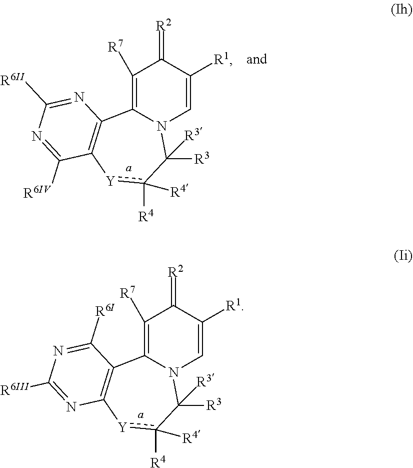

In certain embodiments, the compound of formula (Ia) is selected from the group consisting of:

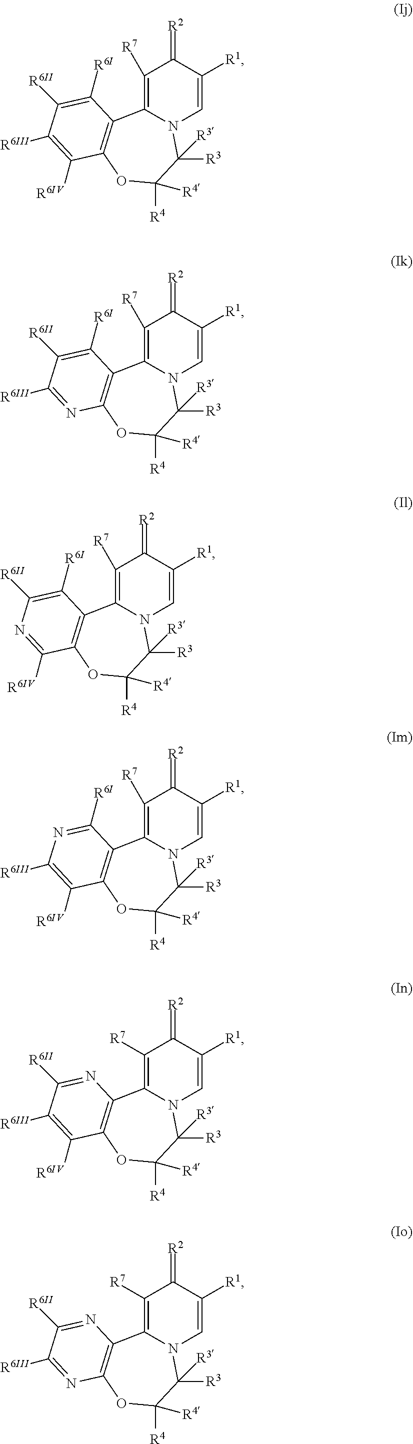

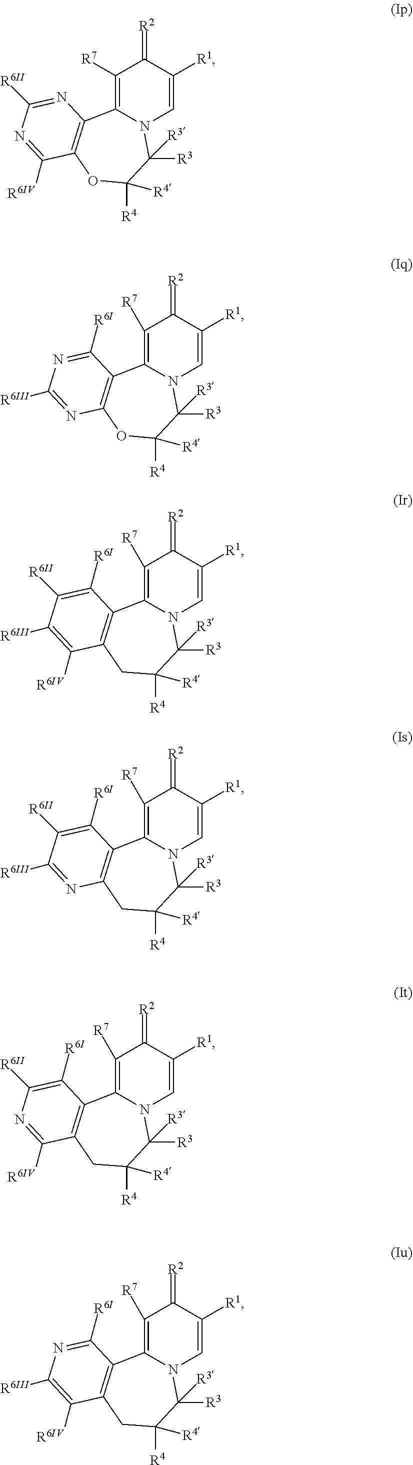

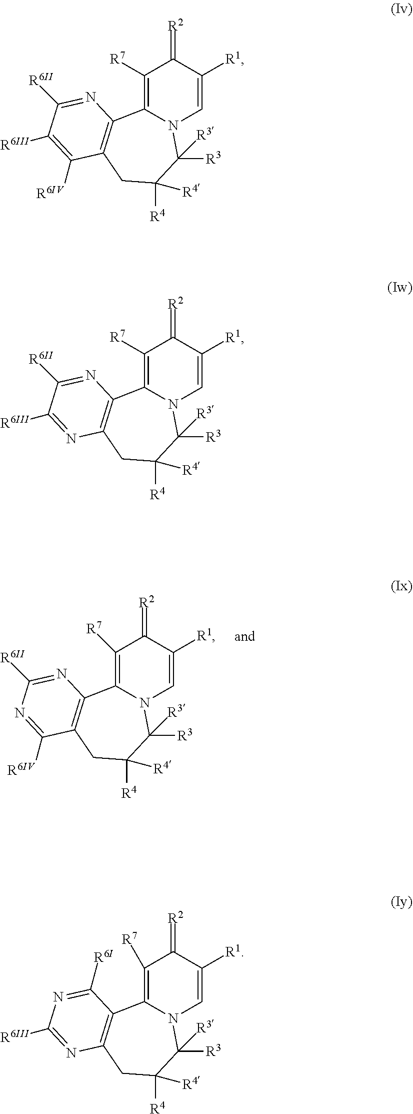

In certain embodiments, the compound of formula (II) is selected from the group consisting of:

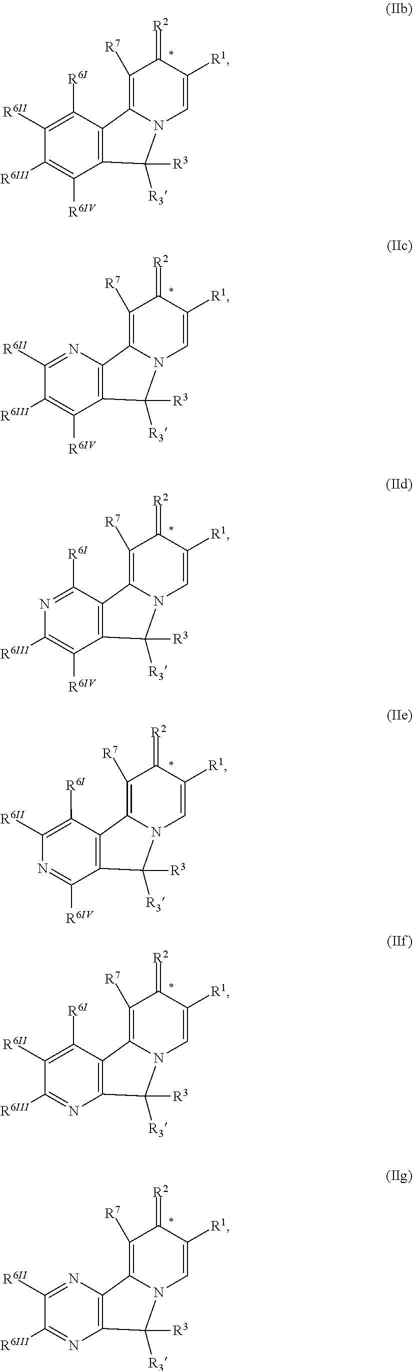

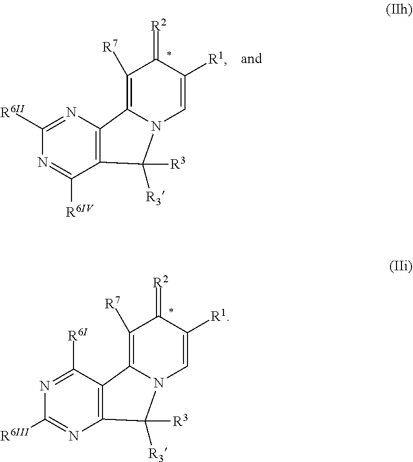

In certain embodiments, the compound of formula (III) is selected from the group consisting of:

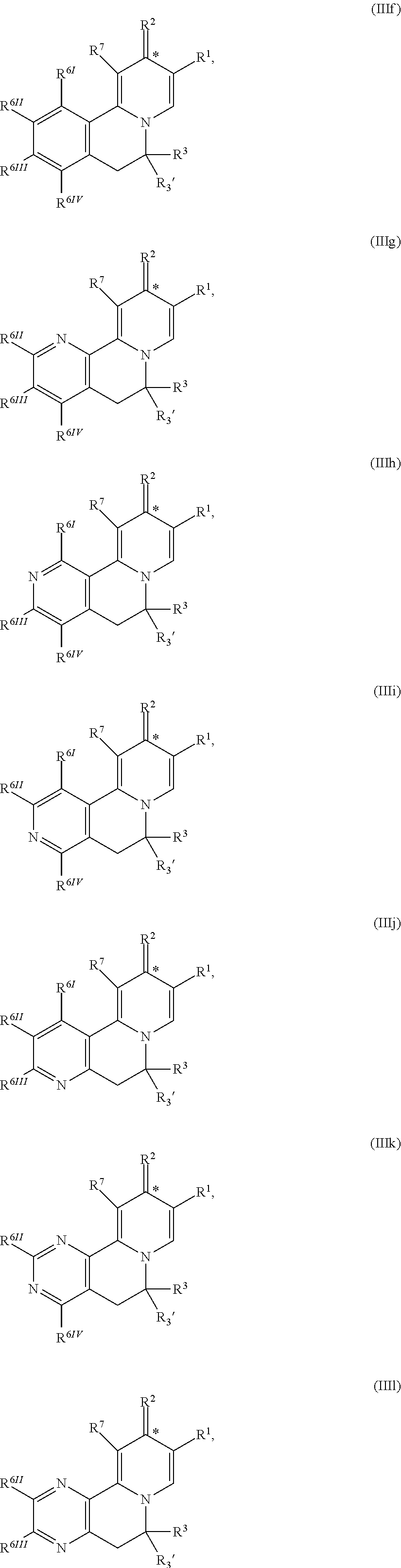

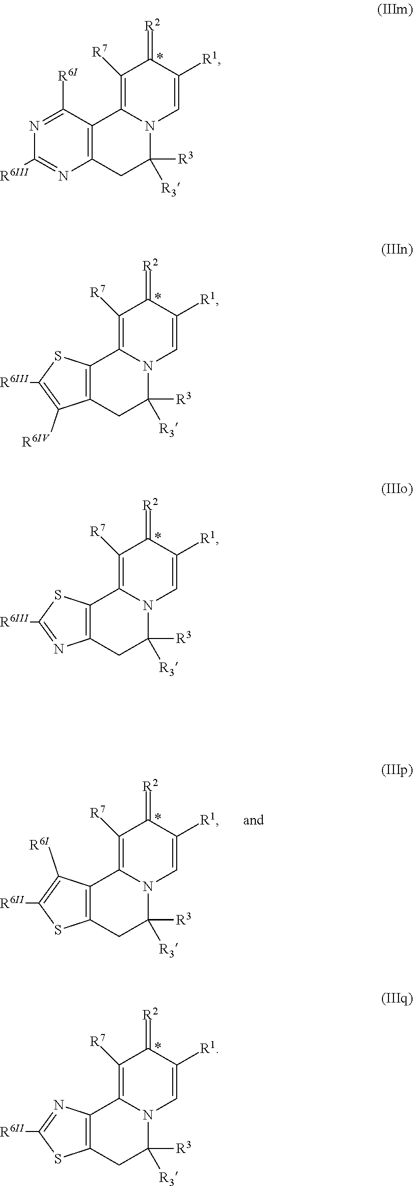

In certain embodiments, a sAg secretion inhibitor/RNA destabilizer is elected from the following compounds, or salts thereof.

| Structure | Nomenclature |
|---|---|
| 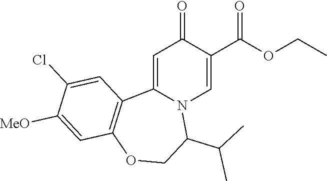 [SMILES: CCOC(=O)c1cn2c(cc1=O)-c1cc(Cl)c(OC)cc1OCC2C(C)C] | ethyl 2-chloro-7-isopropyl-3-methoxy-11-oxo- 6,7-dihydro-11H-benzo[f]pyrido[1,2- d][1,4]oxazepine-10-carboxylate |
| 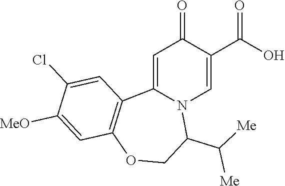 [SMILES: COc1cc2c(cc1Cl)-c1cc(=O)c(C(=O)O)cn1C(C(C)C)CO2] | 2-chloro-7-isopropyl-3-methoxy-11-oxo-6,7- dihydro-11H-benzo[f]pyrido[1,2- d][1,4]oxazepine-10-carboxylic acid |
| 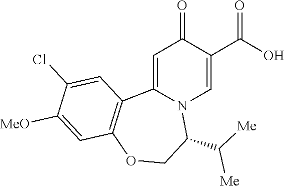 [SMILES: COc1cc2c(cc1Cl)-c1cc(=O)c(C(=O)O)cn1[C@H](C(C)C)CO2] | (R)-2-chloro-7-isopropyl-3-methoxy-11-oxo-6,7- dihydro-11H-benzo[f]pyrido[1,2- d][1,4]oxazepine-10-carboxylic acid |
| 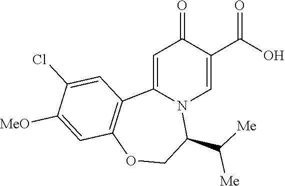 [SMILES: COc1cc2c(cc1Cl)-c1cc(=O)c(C(=O)O)cn1[C@@H](C(C)C)CO2] | (S)-2-chloro-7-isopropyl-3-methoxy-11-oxo-6,7- dihydro-11H-benzo[f]pyrido[1,2- d][1,4]oxazepine-10-carboxylic acid |
| 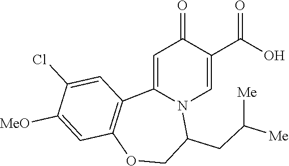 [SMILES: COc1cc2c(cc1Cl)-c1cc(=O)c(C(=O)O)cn1C(CC(C)C)CO2] | 2-chloro-7-isobutyl-3-methoxy-11-oxo-6,7- dihydro-11H-benzo[f]pyrido[1,2- d][1,4]oxazepine-10-carboxylic acid |
| 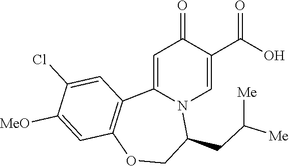 [SMILES: COc1cc2c(cc1Cl)-c1cc(=O)c(C(=O)O)cn1[C@@H](CC(C)C)CO2] | (S)-2-chloro-7-isobutyl-3-methoxy-11-oxo-6,7- dihydro-11H-benzo[f]pyrido[1,2- d][1,4]oxazepine-10-carboxylic acid |
| 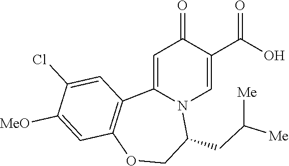 [SMILES: COc1cc2c(cc1Cl)-c1cc(=O)c(C(=O)O)cn1[C@H](CC(C)C)CO2] | (R)-2-chloro-7-isobutyl-3-methoxy-11-oxo-6,7- dihydro-11H-benzo[f]pyrido[1,2- d][1,4]oxazepine-10-carboxylic acid |
| 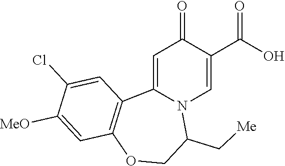 [SMILES: CCC1COc2cc(OC)c(Cl)cc2-c2cc(=O)c(C(=O)O)cn21] | 2-chloro-7-ethyl-3-methoxy-11-oxo-6,7-dihydro- 11H-benzo[f]pyrido[1,2-d][1,4]oxazepine-10- carboxylic acid |
| 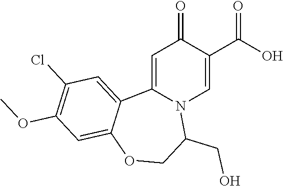 [SMILES: COc1cc2c(cc1Cl)-c1cc(=O)c(C(=O)O)cn1C(CO)CO2] | 2-chloro-7-(hydroxymethyl)-3-methoxy-11-oxo- 6,7-dihydro-11H-benzo[f]pyrido[1,2- d][1,4]oxazepine-10-carboxylic acid |
| 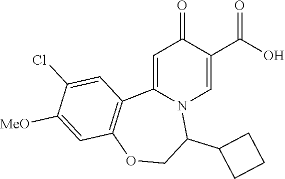 [SMILES: COc1cc2c(cc1Cl)-c1cc(=O)c(C(=O)O)cn1C(C1CCC1)CO2] | 2-chloro-7-cyclobutyl-3-methoxy-11-oxo-6,7- dihydro-11H-benzo[f]pyrido[1,2- d][1,4]oxazepine-10-carboxylic acid |
| 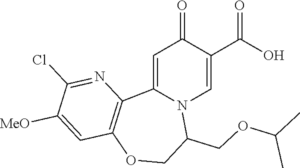 [SMILES: COc1cc2c(nc1Cl)-c1cc(=O)c(C(=O)O)cn1C(COC(C)C)CO2] | 2-chloro-7-(isopropoxymethyl)-3-methoxy-11- oxo-6,7-dihydro-11H-dipyrido[1,2-d:2′,3′- f][1,4]oxazepine-10-carboxylic acid |
| 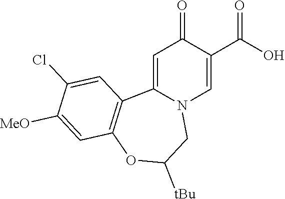 [SMILES: COc1cc2c(cc1Cl)-c1cc(=O)c(C(=O)O)cn1CC(C(C)(C)C)O2] | 6-(tert-butyl)-2-chloro-3-methoxy-11-oxo-6,7- dihydro-11H-benzo[f]pyrido[1,2- d][1,4]oxazepine-10-carboxylic acid |
| 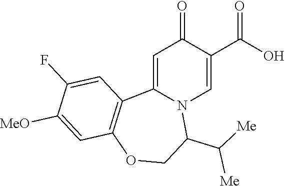 [SMILES: COc1cc2c(cc1F)-c1cc(=O)c(C(=O)O)cn1C(C(C)C)CO2] | 2-fluoro-7-isopropyl-3-methoxy-11-oxo-6,7- dihydro-11H-benzo[f]pyrido[1,2- d][1,4]oxazepine-10-carboxylic acid |
| 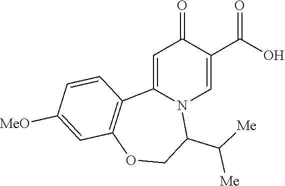 [SMILES: COc1ccc2c(c1)OCC(C(C)C)n1cc(C(=O)O)c(=O)cc1-2] | 7-isopropyl-3-methoxy-11-oxo-6,7-dihydro-11H- benzo[f]pyrido[1,2-d][1,4]oxazepine-10- carboxylic acid |
| 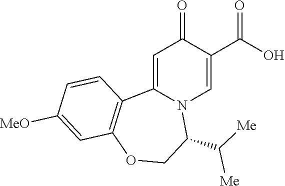 [SMILES: COc1ccc2c(c1)OC[C@@H](C(C)C)n1cc(C(=O)O)c(=O)cc1-2] | (R)-7-isopropyl-3-methoxy-11-oxo-6,7-dihydro- 11H-benzo[f]pyrido[1,2-d][1,4]oxazepine-10- carboxylic acid |
| 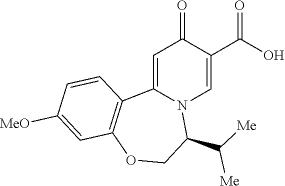 [SMILES: COc1ccc2c(c1)OC[C@H](C(C)C)n1cc(C(=O)O)c(=O)cc1-2] | (S)-7-isopropyl-3-methoxy-11-oxo-6,7-dihydro- 11H-benzo[f]pyrido[1,2-d][1,4]oxazepine-10- carboxylic acid |
| 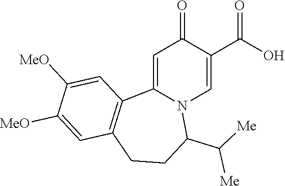 [SMILES: COc1cc2c(cc1OC)-c1cc(=O)c(C(=O)O)cn1C(C(C)C)CC2] | 6-isopropyl-10,11-dimethoxy-2-oxo-2,6,7,8- tetrahydrobenzo[c]pyrido[1,2-a]azepine-3- carboxylic acid |
| 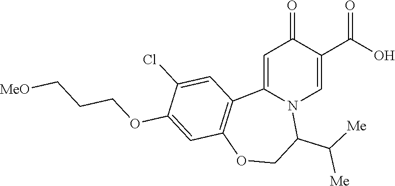 [SMILES: COCCCOc1cc2c(cc1Cl)-c1cc(=O)c(C(=O)O)cn1C(C(C)C)CO2] | 2-chloro-7-isopropyl-3-(3-methoxy propoxy)-11- oxo-6,7-dihydro-11H-benzo[f]pyrido[1,2- d][1,4]oxazepine-10-carboxylic acid |
| 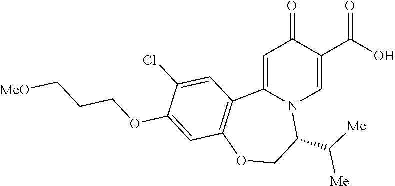 [SMILES: COCCCOc1cc2c(cc1Cl)-c1cc(=O)c(C(=O)O)cn1[C@H](C(C)C)CO2] | (R)-2-chloro-7-isopropyl-3-(3-methoxypropoxy)- 11-oxo-6,7-dihydro-11H-benzo[f]pyrido[1,2- d][1,4]oxazepine-10-carboxylic acid |
| 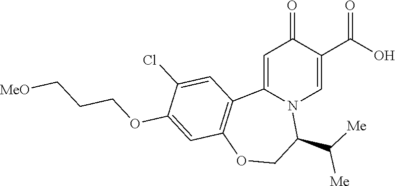 [SMILES: COCCCOc1cc2c(cc1Cl)-c1cc(=O)c(C(=O)O)cn1[C@@H](C(C)C)CO2] | (S)-2-chloro-7-isopropyl-3-(3-methoxypropoxy)- 11-oxo-6,7-dihydro-11H-benzo[f]pyrido[1,2- d][1,4]oxazepine-10-carboxylic acid |
| 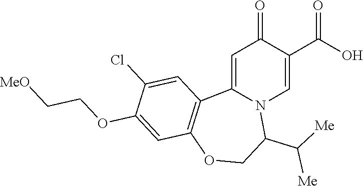 [SMILES: COCCOc1cc2c(cc1Cl)-c1cc(=O)c(C(=O)O)cn1C(C(C)C)CO2] | 2-chloro-7-isopropyl-3-(2-methoxy ethoxy)-11- oxo-6,7-dihydro-11H-benzo[f]pyrido[1,2- d][1,4]oxazepine-10-carboxylic acid |
| 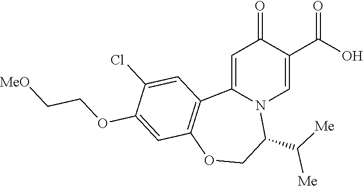 [SMILES: COCCOc1cc2c(cc1Cl)-c1cc(=O)c(C(=O)O)cn1[C@H](C(C)C)CO2] | (R)-2-chloro-7-isopropyl-3-(2-methoxyethoxy)- 11-oxo-6,7-dihydro-11H-benzo[f]pyrido[1,2- d][1,4]oxazepine-10-carboxylic acid |
| 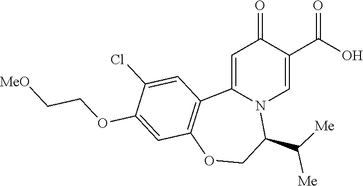 [SMILES: COCCOc1cc2c(cc1Cl)-c1cc(=O)c(C(=O)O)cn1[C@@H](C(C)C)CO2] | (S)-2-chloro-7-isopropyl-3-(2-methoxyethoxy)- 11-oxo-6,7-dihydro-11H-benzo[f]pyrido[1,2- d][1,4]oxazepine-10-carboxylic acid |
| 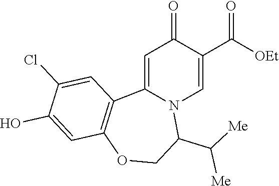 [SMILES: CCOC(=O)c1cn2c(cc1=O)-c1cc(Cl)c(O)cc1OCC2C(C)C] | ethyl 2-chloro-3-hydroxy-7-isopropyl-11-oxo-6,7- dihydro-11H-benzo[f]pyrido[1,2- d][1,4]oxazepine-10-carboxylate |
| 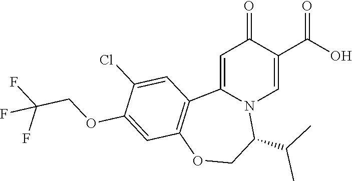 [SMILES: CC(C)[C@@H]1COc2cc(OCC(F)(F)F)c(Cl)cc2-c2cc(=O)c(C(=O)O)cn21] | (R)-2-chloro-7-isopropyl-11-oxo-3-(2,2,2- trifluoroethoxy)-6,7-dihydro-11H- benzo[f]pyrido[1,2-d][1,4]oxazepine-10- carboxylic acid |
| 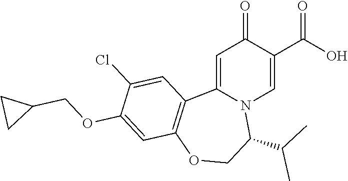 [SMILES: CC(C)[C@@H]1COc2cc(OCC3CC3)c(Cl)cc2-c2cc(=O)c(C(=O)O)cn21] | (R)-2-chloro-3-(cyclopropylmethoxy)-7- isopropyl-11-oxo-6,7-dihydro-11H- benzo[f]pyrido[1,2-d][1,4]oxazepine-10- carboxylic acid |
| 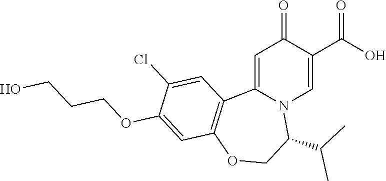 [SMILES: CC(C)[C@@H]1COc2cc(OCCCO)c(Cl)cc2-c2cc(=O)c(C(=O)O)cn21] | (R)-2-chloro-3-(3-hydroxypropoxy)-7-isopropyl- 11-oxo-6,7-dihydro-11H-benzo[f]pyrido[1,2- d][1,4]oxazepine-10-carboxylic acid |
| 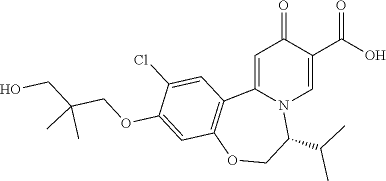 [SMILES: CC(C)[C@@H]1COc2cc(OCC(C)(C)CO)c(Cl)cc2-c2cc(=O)c(C(=O)O)cn21] | (R)-2-chloro-3-(3-hydroxy-2,2-dimethylpropoxy)- 7-isopropyl-11-oxo-6,7-dihydro-11H- benzo[f]pyrido[1,2-d][1,4]oxazepine-10- carboxylic acid |
| 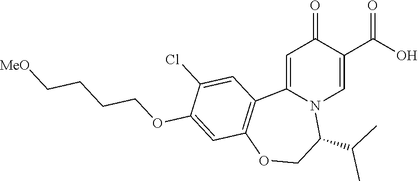 [SMILES: COCCCCOc1cc2c(cc1Cl)-c1cc(=O)c(C(=O)O)cn1[C@H](C(C)C)CO2] | (R)-2-chloro-7-isopropyl-3-(4-methoxybutoxy)- 11-oxo-6,7-dihydro-11H-benzo[f]pyrido[1,2- d][1,4]oxazepine-10-carboxylic acid |
| 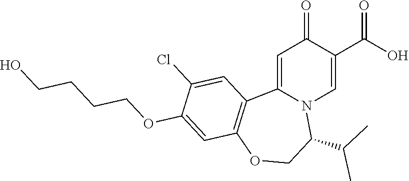 [SMILES: CC(C)[C@@H]1COc2cc(OCCCCO)c(Cl)cc2-c2cc(=O)c(C(=O)O)cn21] | (R)-2-chloro-3-(4-hydroxybutoxy)-7-isopropyl- 11-oxo-6,7-dihydro-11H-benzo[f]pyrido[1,2- d][1,4]oxazepine-10-carboxylic acid |
| 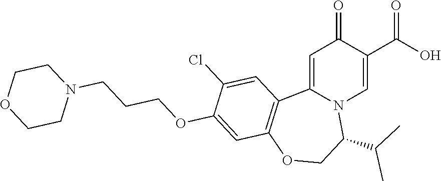 [SMILES: CC(C)[C@@H]1COc2cc(OCCCN3CCOCC3)c(Cl)cc2-c2cc(=O)c(C(=O)O)cn21] | (R)-2-chloro-7-isopropyl-3-(3- morpholinopropoxy)-11-oxo-6,7-dihydro-11H- benzo[f]pyrido[1,2-d][1,4]oxazepine-10- carboxylic acid |
| 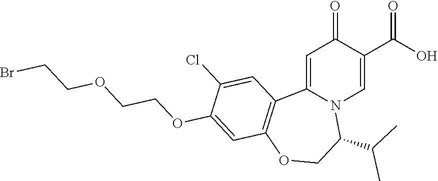 [SMILES: CC(C)[C@@H]1COc2cc(OCCOCCBr)c(Cl)cc2-c2cc(=O)c(C(=O)O)cn21] | (R)-3-(2-(2-bromoethoxy)ethoxy)-2-chloro-7- isopropyl-11-oxo-6,7-dihydro-11H- benzo[f]pyrido[1,2-d][1,4]oxazepine-10- carboxylic acid |
| 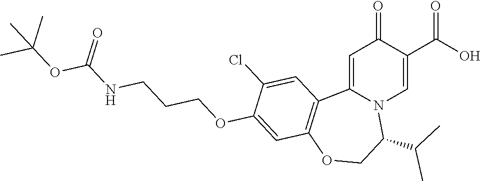 [SMILES: CC(C)[C@@H]1COc2cc(OCCCNC(=O)OC(C)(C)C)c(Cl)cc2-c2cc(=O)c(C(=O)O)cn21] | (R)-3-(3-((tert-butoxycarbonyl)amino)propoxy)- 2-chloro-7-isopropyl-11-oxo-6,7-dihydro-11H- benzo[f]pyrido[1,2-d][1,4]oxazepine-10- carboxylic acid |
| 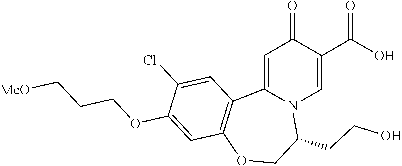 [SMILES: COCCCOc1cc2c(cc1Cl)-c1cc(=O)c(C(=O)O)cn1[C@H](CCO)CO2] | (R)-2-chloro-7-(2-hydroxyethyl)-3-(3- methoxypropoxy)-11-oxo-6,7-dihydro-11H- benzo[f]pyrido[1,2-d][1,4]oxazepine-10- carboxylic acid |
| 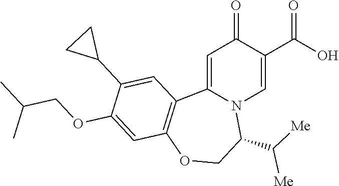 [SMILES: CC(C)COc1cc2c(cc1C1CC1)-c1cc(=O)c(C(=O)O)cn1[C@H](C(C)C)CO2] | (R)-2-cyclopropyl-3-isobutoxy-7-isopropyl-11- oxo-6,7-dihydro-11H-benzo[f]pyrido[1,2- d][1,4]oxazepine-10-carboxylic acid |
| 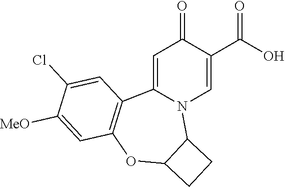 [SMILES: COc1cc2c(cc1Cl)-c1cc(=O)c(C(=O)O)cn1C1CCC1O2] | 11-chloro-10-methoxy-2-oxo-5a,6,7,7a- tetrahydro-2H-benzo[f]cyclobuta[b]pyrido[1,2- d][1,4]oxazepine-3-carboxylic acid |
| 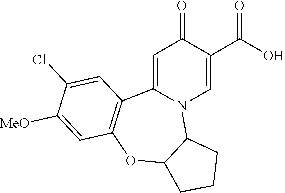 [SMILES: COc1cc2c(cc1Cl)-c1cc(=O)c(C(=O)O)cn1C1CCCC1O2] | 12-chloro-11-methoxy-2-oxo-5a,7,8,8a- tetrahydro-2H,6H- benzo[f]cyclopenta[b]pyrido[1,2- d][1,4]oxazepine-3-carboxylic acid |
| 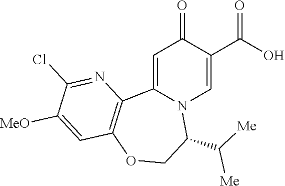 [SMILES: COc1cc2c(nc1Cl)-c1cc(=O)c(C(=O)O)cn1[C@H](C(C)C)CO2] | (R)-2-chloro-7-isopropyl-3-methoxy-11-oxo-6,7- dihydro-11H-dipyrido[1,2-d:2′,3′- f][1,4]oxazepine-10-carboxylic acid |
| 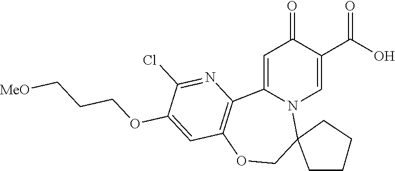 [SMILES: COCCCOc1cc2c(nc1Cl)-c1cc(=O)c(C(=O)O)cn1C1(CCCC1)CO2] | 2′-chloro-3′-(3-methoxypropoxy)-11′-oxo- 6′H,11′H-spiro[cyclopentane-1,7′-dipyrido[1,2- d:2′,3′-f][1,4]oxazepine]-10′-carboxylic acid |
| 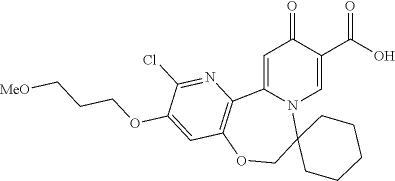 [SMILES: COCCCOc1cc2c(nc1Cl)-c1cc(=O)c(C(=O)O)cn1C1(CCCCC1)CO2] | 2′-chloro-3′-(3-methoxypropoxy)-11′-oxo- 6′H,11′H-spiro[cyclohexane-1,7′-dipyrido[1,2- d:2′,3′-f][1,4]oxazepine]-10′-carboxylic acid |
| 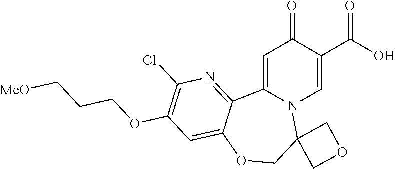 [SMILES: COCCCOc1cc2c(nc1Cl)-c1cc(=O)c(C(=O)O)cn1C1(COC1)CO2] | 2-chloro-3-(3-methoxypropoxy)-11-oxo-6H,11H- spiro[dipyrido[1,2-d:2′,3′-f][1,4]oxazepine-7,3′- oxetane]-10-carboxylic acid |
| 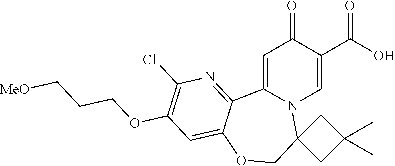 [SMILES: COCCCOc1cc2c(nc1Cl)-c1cc(=O)c(C(=O)O)cn1C1(CO2)CC(C)(C)C1] | 2′-chloro-3′-(3-methoxypropoxy)-3,3-dimethyl- 11′-oxo-6′H,11′H-spiro[cyclobutane-1,7′- dipyrido[1,2-d:2′,3′-f][1,4]oxazepine]-10′- carboxylic acid |
| 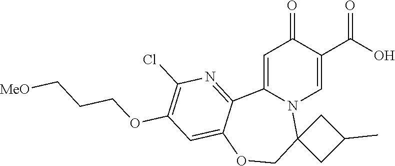 [SMILES: COCCCOc1cc2c(nc1Cl)-c1cc(=O)c(C(=O)O)cn1C1(CO2)CC(C)C1] | 2′-chloro-3′-(3-methoxypropoxy)-3-methyl-11′- oxo-6′H,11′H-spiro[cyclobutane-1,7′- dipyrido[1,2-d:2′,3′-f][1,4]oxazepine]-10′- carboxylic acid |
| 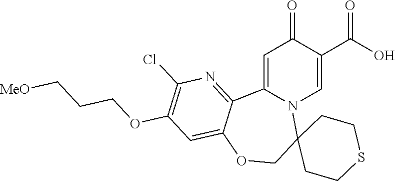 [SMILES: COCCCOc1cc2c(nc1Cl)-c1cc(=O)c(C(=O)O)cn1C1(CCSCC1)CO2] | 2-chloro-3-(3-methoxypropoxy)-11-oxo- 2′,3′,5′,6′-tetrahydro-6H,11H-spiro[dipyrido[1,2- d:2′,3′-f][1,4]oxazepine-7,4′-thiopyran]-10- carboxylic acid |
| 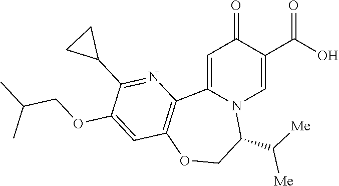 [SMILES: CC(C)COc1cc2c(nc1C1CC1)-c1cc(=O)c(C(=O)O)cn1[C@H](C(C)C)CO2] | (R)-2-cyclopropyl-3-isobutoxy-7-isopropyl-11- oxo-6,7-dihydro-11H-dipyrido[1,2-d:2′,3′- f][1,4]oxazepine-10-carboxylic acid |
| 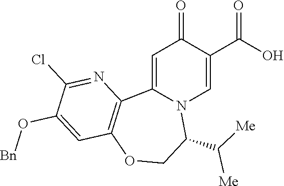 [SMILES: CC(C)[C@@H]1COc2cc(OCc3ccccc3)c(Cl)nc2-c2cc(=O)c(C(=O)O)cn21] | (R)-3-(benzyloxy)-2-chloro-7-isopropyl-11-oxo- 6,7-dihydro-11H-dipyrido[1,2-d:2′,3′- f][1,4]oxazepine-10-carboxylic acid |
| 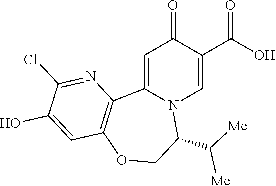 [SMILES: CC(C)[C@@H]1COc2cc(O)c(Cl)nc2-c2cc(=O)c(C(=O)O)cn21] | (R)-2-chloro-3-hydroxy-7-isopropyl-11-oxo-6,7- dihydro-11H-dipyrido[1,2-d:2′,3′- f][1,4]oxazepine-10-carboxylic acid |
| 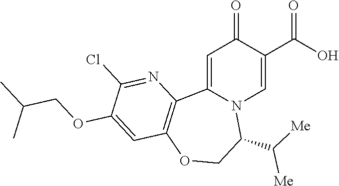 [SMILES: CC(C)COc1cc2c(nc1Cl)-c1cc(=O)c(C(=O)O)cn1[C@H](C(C)C)CO2] | (R)-2-chloro-3-isobutoxy-7-isopropyl-11-oxo-6,7- dihydro-11H-dipyrido[1,2-d:2′,3′- f][1,4]oxazepine-10-carboxylic acid |
| 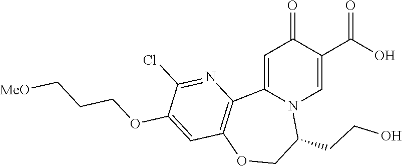 [SMILES: COCCCOc1cc2c(nc1Cl)-c1cc(=O)c(C(=O)O)cn1[C@H](CCO)CO2] | (R)-2-chloro-7-(2-hydroxyethyl)-3-(3- methoxypropoxy)-11-oxo-6,7-dihydro-11H- dipyrido[1,2-d:2′,3′-f][1,4]oxazepine-10- carboxylic acid |
| 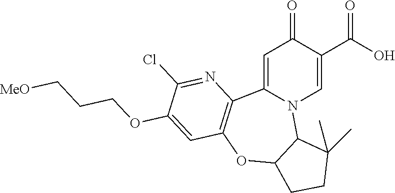 [SMILES: COCCCOc1cc2c(nc1Cl)-c1cc(=O)c(C(=O)O)cn1C1C(CCC1(C)C)O2] | 6-chloro-7-(3-methoxypropoxy)-12,12-dimethyl- 3-oxo-9a,11,12,12a-tetrahydro-3H,10H- cyclopenta[b]dipyrido[1,2-d:2′,3′- f][1,4]oxazepine-2-carboxylic acid |
| 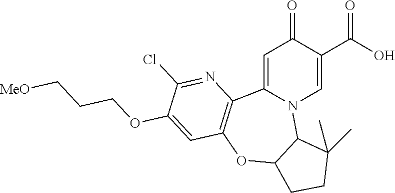 [SMILES: COCCCOc1cc2c(nc1Cl)-c1cc(=O)c(C(=O)O)cn1C1C(CCC1(C)C)O2] | 6-chloro-7-(3-methoxypropoxy)-12,12-dimethyl- 3-oxo-9a,11,12,12a-tetrahydro-3H,10H- cyclopenta[b]dipyrido[1,2-d:2′,3′- f][1,4]oxazepine-2-carboxylic acid (single enantiomer I) |
| 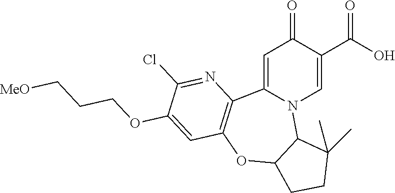 [SMILES: COCCCOc1cc2c(nc1Cl)-c1cc(=O)c(C(=O)O)cn1C1C(CCC1(C)C)O2] | 6-chloro-7-(3-methoxypropoxy)-12,12-dimethyl- 3-oxo-9a,11,12,12a-tetrahydro-3H,10H- cyclopenta[b]dipyrido[1,2-d:2′,3′- f][1,4]oxazepine-2-carboxylic acid (single enantiomer II) |
| 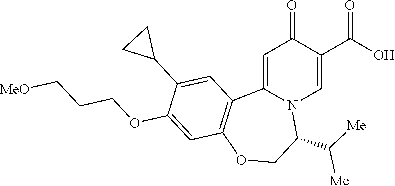 [SMILES: COCCCOc1cc2c(cc1C1CC1)-c1cc(=O)c(C(=O)O)cn1[C@H](C(C)C)CO2] | (R)-2-cyclopropyl-7-isopropyl-3-(3- methoxypropoxy)-11-oxo-6,7-dihydro-11H- benzo[f]pyrido[1,2-d][1,4]oxazepine-10- carboxylic acid |
| 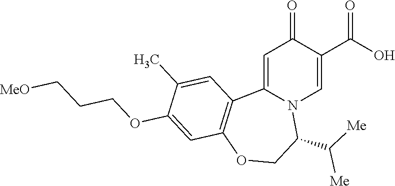 [SMILES: COCCCOc1cc2c(cc1C)-c1cc(=O)c(C(=O)O)cn1[C@H](C(C)C)CO2] | (R)-7-isopropyl-3-(3-methoxypropoxy)-2-methyl- 11-oxo-6,7-dihydro-11H-benzo[f]pyrido[1,2- d][1,4]oxazepine-10-carboxylic acid |
| 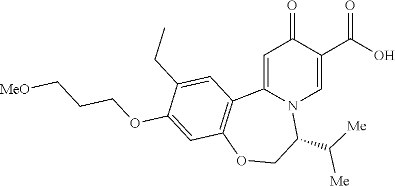 [SMILES: CCc1cc2c(cc1OCCCOC)OC[C@@H](C(C)C)n1cc(C(=O)O)c(=O)cc1-2] | (R)-2-ethyl-7-isopropyl-3-(3-methoxypropoxy)- 11-oxo-6,7-dihydro-11H-benzo[f]pyrido[1,2- d][1,4]oxazepine-10-carboxylic acid |
| 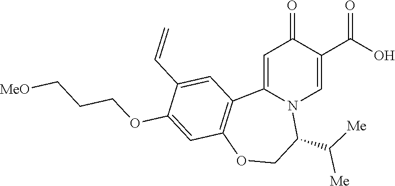 [SMILES: C=Cc1cc2c(cc1OCCCOC)OC[C@@H](C(C)C)n1cc(C(=O)O)c(=O)cc1-2] | (R)-7-isopropyl-3-(3-methoxypropoxy)-11-oxo-2- vinyl-6,7-dihydro-11H-benzo[f]pyrido[1,2- d][1,4]oxazepine-10-carboxylic acid |
| 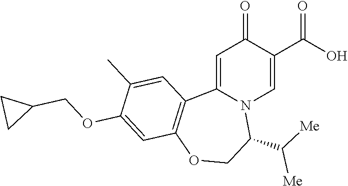 [SMILES: Cc1cc2c(cc1OCC1CC1)OC[C@@H](C(C)C)n1cc(C(=O)O)c(=O)cc1-2] | (R)-3-(cyclopropylmethoxy)-7-isopropyl-2- methyl-11-oxo-6,7-dihydro-11H- benzo[f]pyrido[1,2-d][1,4]oxazepine-10- carboxylic acid |
| 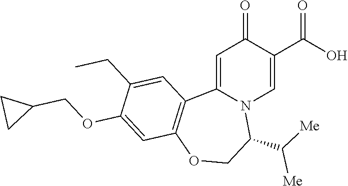 [SMILES: CCc1cc2c(cc1OCC1CC1)OC[C@@H](C(C)C)n1cc(C(=O)O)c(=O)cc1-2] | (R)-3-(cyclopropylmethoxy)-2-ethyl-7-isopropyl- 11-oxo-6,7-dihydro-11H-benzo[f]pyrido[1,2- d][1,4]oxazepine-10-carboxylic acid |
| 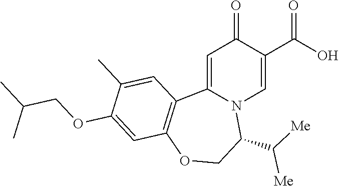 [SMILES: Cc1cc2c(cc1OCC(C)C)OC[C@@H](C(C)C)n1cc(C(=O)O)c(=O)cc1-2] | (R)-3-isobutoxy-7-isopropyl-2-methyl-11-oxo- 6,7-dihydro-11H-benzo[f]pyrido[1,2- d][1,4]oxazepine-10-carboxylic acid |
| 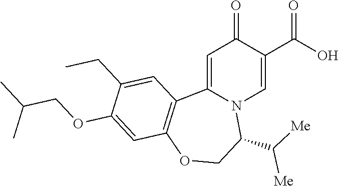 [SMILES: CCc1cc2c(cc1OCC(C)C)OC[C@@H](C(C)C)n1cc(C(=O)O)c(=O)cc1-2] | (R)-2-ethyl-3-isobutoxy-7-isopropyl-11-oxo-6,7- dihydro-11H-benzo[f]pyrido[1,2- d][1,4]oxazepine-10-carboxylic acid |
| 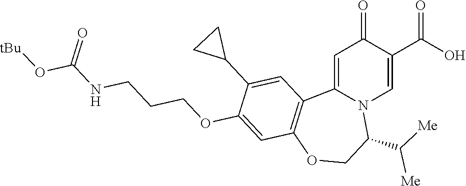 [SMILES: CC(C)[C@@H]1COc2cc(OCCCNC(=O)OC(C)(C)C)c(C3CC3)cc2-c2cc(=O)c(C(=O)O)cn21] | (R)-3-(3-((tert-butoxycarbonyl)amino)propoxy)- 2-cyclopropyl-7-isopropyl-11-oxo-6,7-dihydro- 11H-benzo[f]pyrido[1,2-d][1,4]oxazepine-10- carboxylic acid |
| 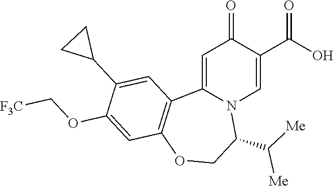 [SMILES: CC(C)[C@@H]1COc2cc(OCC(F)(F)F)c(C3CC3)cc2-c2cc(=O)c(C(=O)O)cn21] | (R)-2-cyclopropyl-7-isopropyl-11-oxo-3-(2,2,2- trifluoroethoxy)-6,7-dihydro-11H- benzo[f]pyrido[1,2-d][1,4]oxazepine-10- carboxylic acid |
| 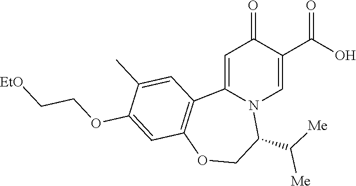 [SMILES: CCOCCOc1cc2c(cc1C)-c1cc(=O)c(C(=O)O)cn1[C@H](C(C)C)CO2] | (R)-3-(2-ethoxyethoxy)-7-isopropyl-2-methyl-11- oxo-6,7-dihydro-11H-benzo[f]pyrido[1,2- d][1,4]oxazepine-10-carboxylic acid |
| 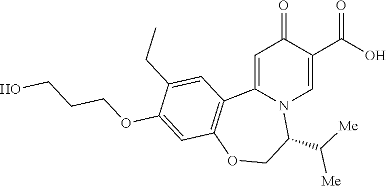 [SMILES: CCc1cc2c(cc1OCCCO)OC[C@@H](C(C)C)n1cc(C(=O)O)c(=O)cc1-2] | (R)-2-ethyl-3-(3-hydroxypropoxy)-7-isopropyl- 11-oxo-6,7-dihydro-11H-benzo[f]pyrido[1,2- d][1,4]oxazepine-10-carboxylic acid |
| 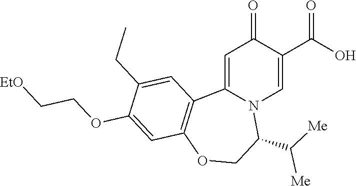 [SMILES: CCOCCOc1cc2c(cc1CC)-c1cc(=O)c(C(=O)O)cn1[C@H](C(C)C)CO2] | (R)-3-(2-ethoxyethoxy)-2-ethyl-7-isopropyl-11- oxo-6,7-dihydro-11H-benzo[f]pyrido[1,2- d][1,4]oxazepine-10-carboxylic acid |
| 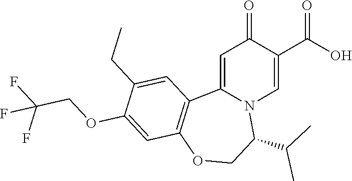 [SMILES: CCc1cc2c(cc1OCC(F)(F)F)OC[C@@H](C(C)C)n1cc(C(=O)O)c(=O)cc1-2] | (R)-2-ethyl-7-isopropyl-11-oxo-3-(2,2,2- trifluoroethoxy)-6,7-dihydro-11H- benzo[f]pyrido[1,2-d][1,4]oxazepine-10- carboxylic acid |
| 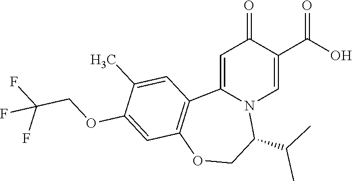 [SMILES: Cc1cc2c(cc1OCC(F)(F)F)OC[C@@H](C(C)C)n1cc(C(=O)O)c(=O)cc1-2] | (R)-7-isopropyl-2-methyl-11-oxo-3-(2,2,2- trifluoroethoxy)-6,7-dihydro-11H- benzo[f]pyrido[1,2-d][1,4]oxazepine-10- carboxylic acid |
| 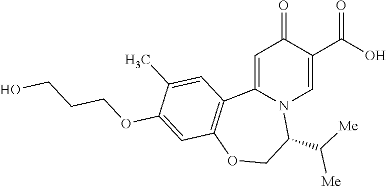 [SMILES: Cc1cc2c(cc1OCCCO)OC[C@@H](C(C)C)n1cc(C(=O)O)c(=O)cc1-2] | (R)-3-(3-hydroxypropoxy)-7-isopropyl-2-methyl- 11-oxo-6,7-dihydro-11H-benzo[f]pyrido[1,2- d][1,4]oxazepine-10-carboxylic acid |
| 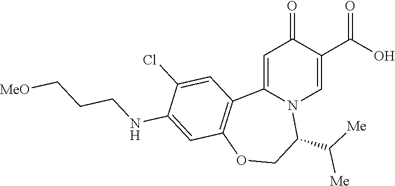 [SMILES: COCCCNc1cc2c(cc1Cl)-c1cc(=O)c(C(=O)O)cn1[C@H](C(C)C)CO2] | (R)-2-chloro-7-isopropyl-3-((3- methoxypropyl)amino)-11-oxo-6,7-dihydro-11H- benzo[f]pyrido[1,2-d][1,4]oxazepine-10- carboxylic acid |
| 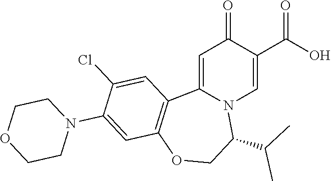 [SMILES: CC(C)[C@@H]1COc2cc(N3CCOCC3)c(Cl)cc2-c2cc(=O)c(C(=O)O)cn21] | (R)-2-chloro-7-isopropyl-3-morpholino-11-oxo- 6,7-dihydro-11H-benzo[f]pyrido[1,2- d][1,4]oxazepine-10-carboxylic acid |
| 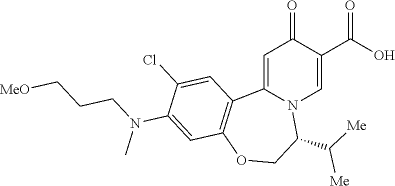 [SMILES: COCCCN(C)c1cc2c(cc1Cl)-c1cc(=O)c(C(=O)O)cn1[C@H](C(C)C)CO2] | (R)-2-chloro-7-isopropyl-3-((3- methoxypropyl)(methyl)amino)-11-oxo-6,7- dihydro-11H-benzo[f]pyrido[1,2- d][1,4]oxazepine-10-carboxylic acid |
| 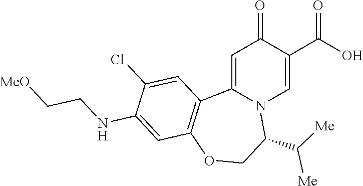 [SMILES: COCCNc1cc2c(cc1Cl)-c1cc(=O)c(C(=O)O)cn1[C@H](C(C)C)CO2] | (R)-2-chloro-7-isopropyl-3-((2- methoxyethyl)amino)-11-oxo-6,7-dihydro-11H- benzo[f]pyrido[1,2-d][1,4]oxazepine-10- carboxylic acid |
| 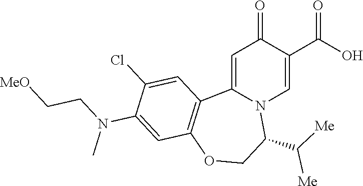 [SMILES: COCCN(C)c1cc2c(cc1Cl)-c1cc(=O)c(C(=O)O)cn1[C@H](C(C)C)CO2] | (R)-2-chloro-7-isopropyl-3-((2- methoxyethyl)(methyl)amino)-11-oxo-6,7- dihydro-11H-benzo[f]pyrido[1,2- d][1,4]oxazepine-10-carboxylic acid |
| 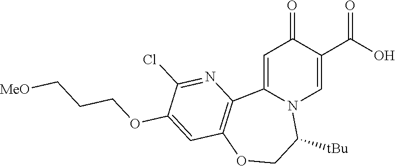 [SMILES: COCCCOc1cc2c(nc1Cl)-c1cc(=O)c(C(=O)O)cn1[C@H](C(C)(C)C)CO2] | (R)-7-(tert-butyl)-2-chloro-3-(3- methoxypropoxy)-11-oxo-6,7-dihydro-11H- dipyrido[1,2-d:2′,3′-f][1,4]oxazepine-10- carboxylic acid |
| 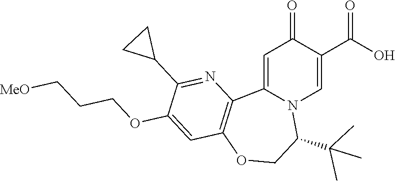 [SMILES: COCCCOc1cc2c(nc1C1CC1)-c1cc(=O)c(C(=O)O)cn1[C@H](C(C)(C)C)CO2] | (R)-7-(tert-butyl)-2-cyclopropyl-3-(3- methoxypropoxy)-11-oxo-6,7-dihydro-11H- dipyrido[1,2-d:2′,3′-f][1,4]oxazepine-10- carboxylic acid |
| 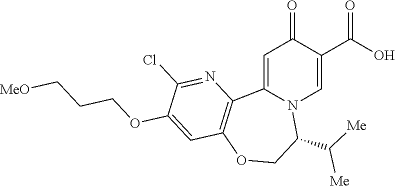 [SMILES: COCCCOc1cc2c(nc1Cl)-c1cc(=O)c(C(=O)O)cn1[C@H](C(C)C)CO2] | (R)-2-chloro-7-isopropyl-3-(3-methoxypropoxy)- 11-oxo-6,7-dihydro-11H-dipyrido[1,2-d:2′,3′- f][1,4]oxazepine-10-carboxylic acid |
| 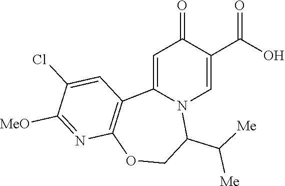 [SMILES: COc1nc2c(cc1Cl)-c1cc(=O)c(C(=O)O)cn1C(C(C)C)CO2] | 2-chloro-7-isopropyl-3-methoxy-11-oxo-6,7- dihydro-11H-dipyrido[1,2-d:3′,2′- f][1,4]oxazepine-10-carboxylic acid |
| 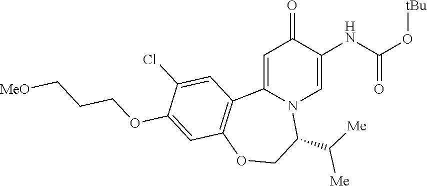 [SMILES: COCCCOc1cc2c(cc1Cl)-c1cc(=O)c(NC(=O)OC(C)(C)C)cn1[C@H](C(C)C)CO2] | tert-butyl (R)-(2-chloro-7-isopropyl-3-(3- methoxypropoxy)-11-oxo-6,7-dihydro-11H- benzo[f]pyrido[1,2-d][1,4]oxazepin-10- yl)carbamate |
| 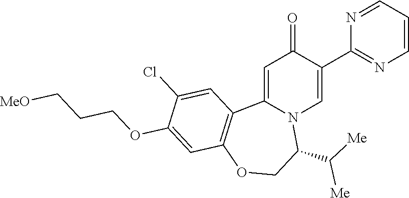 [SMILES: COCCCOc1cc2c(cc1Cl)-c1cc(=O)c(-c3ncccn3)cn1[C@H](C(C)C)CO2] | (R)-2-chloro-7-isopropyl-3-(3-methoxypropoxy)- 10-(pyrimidin-2-yl)-6,7-dihydro-11H- benzo[f]pyrido[1,2-d][1,4]oxazepin-11-one |
| 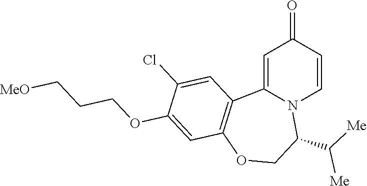 [SMILES: COCCCOc1cc2c(cc1Cl)-c1cc(=O)ccn1[C@H](C(C)C)CO2] | (R)-2-chloro-7-isopropyl-3-(3-methoxypropoxy)- 6,7-dihydro-11H-benzo[f]pyrido[1,2- d][1,4]oxazepin-11-one |
| 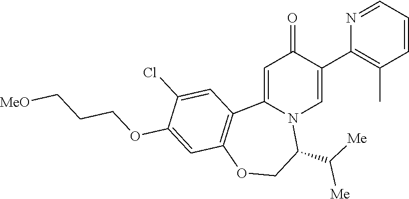 [SMILES: COCCCOc1cc2c(cc1Cl)-c1cc(=O)c(-c3ncccc3C)cn1[C@H](C(C)C)CO2] | (R)-2-chloro-7-isopropyl-3-(3-methoxypropoxy)- 10-(3-methylpyridin-2-yl)-6,7-dihydro-11H- benzo[f]pyrido[1,2-d][1,4]oxazepin-11-one |
| 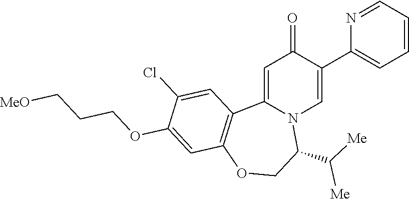 [SMILES: COCCCOc1cc2c(cc1Cl)-c1cc(=O)c(-c3ccccn3)cn1[C@H](C(C)C)CO2] | (R)-2-chloro-7-isopropyl-3-(3-methoxypropoxy)- 10-(pyridin-2-yl)-6,7-dihydro-11H- benzo[f]pyrido[1,2-d][1,4]oxazepin-11-one |
| 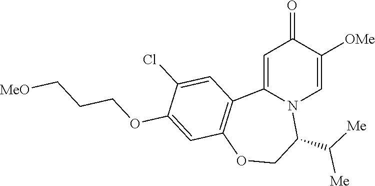 [SMILES: COCCCOc1cc2c(cc1Cl)-c1cc(=O)c(OC)cn1[C@H](C(C)C)CO2] | (R)-2-chloro-7-isopropyl-10-methoxy-3-(3- methoxypropoxy)-6,7-dihydro-11H- benzo[f]pyrido[1,2-d][1,4]oxazepin-11-one |
| 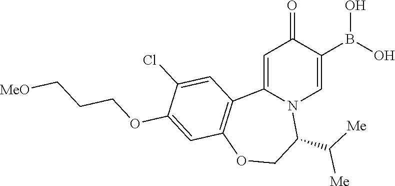 [SMILES: COCCCOc1cc2c(cc1Cl)-c1cc(=O)c(B(O)O)cn1[C@H](C(C)C)CO2] | (R)-(2-chloro-7-isopropyl-3-(3-methoxypropoxy)- 11-oxo-6,7-dihydro-11H-benzo[f]pyrido[1,2- d][1,4]oxazepin-10-yl)boronic acid |
| 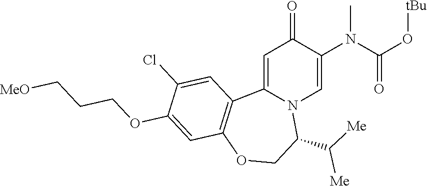 [SMILES: COCCCOc1cc2c(cc1Cl)-c1cc(=O)c(N(C)C(=O)OC(C)(C)C)cn1[C@H](C(C)C)CO2] | tert-butyl (R)-(2-chloro-7-isopropyl-3-(3- methoxypropoxy)-11-oxo-6,7-dihydro-11H- benzo[f]pyrido[1,2-d][1,4]oxazepin-10- yl)(methyl)carbamate |
| 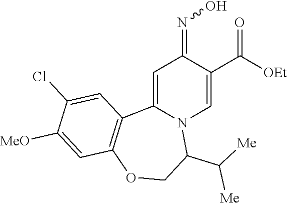 [SMILES: CCOC(=O)c1cn2c(cc1=NO)-c1cc(Cl)c(OC)cc1OCC2C(C)C] | ethyl 2-chloro-11-(hydroxyimino)-7-isopropyl-3- methoxy-6,7-dihydro-11H-benzo[f]pyrido[1,2- d][1,4]oxazepine-10-carboxylate |
| 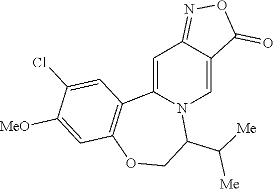 [SMILES: COc1cc2c(cc1Cl)-c1cc3noc(=O)c-3cn1C(C(C)C)CO2] | 2-chloro-7-isopropyl-3-methoxy-6,7-dihydro- 10H-benzo[f]isoxazolo[3′,4′:4,5]pyrido[1,2- d][1,4]oxazepin-10-one |
| 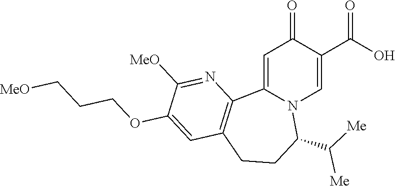 [SMILES: COCCCOc1cc2c(nc1OC)-c1cc(=O)c(C(=O)O)cn1[C@H](C(C)C)CC2] | (S)-7-isopropyl-2-methoxy-3-(3- methoxypropoxy)-11-oxo-5,6,7,11- tetrahydrodipyrido[1,2-a:2′,3′-c]azepine-10- carboxylic acid |
| 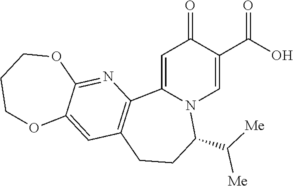 [SMILES: CC(C)[C@@H]1CCc2cc3c(nc2-c2cc(=O)c(C(=O)O)cn21)OCCCO3] | (S)-6-isopropyl-2-oxo-2,6,7,8,12,13-hexahydro- 11H-[1,4]dioxepino[2′,3′:5,6]pyrido[2,3- c]pyrido[1,2-a]azepine-3-carboxylic acid |
| 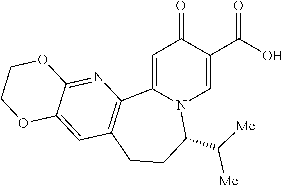 [SMILES: CC(C)[C@@H]1CCc2cc3c(nc2-c2cc(=O)c(C(=O)O)cn21)OCCO3] | (S)-6-isopropyl-2-oxo-2,6,7,8,11,12-hexahydro- [1,4]dioxino[2′,3′:5,6]pyrido[2,3-c]pyrido[1,2- a]azepine-3-carboxylic acid |
| 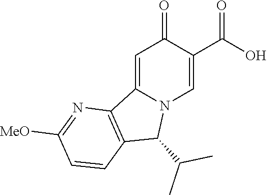 [SMILES: COc1ccc2c(n1)-c1cc(=O)c(C(=O)O)cn1[C@@H]2C(C)C] | (R)-5-isopropyl-2-methoxy-9-oxo-5,9- dihydropyrido[2,3-a]indolizine-8-carboxylic acid |
| 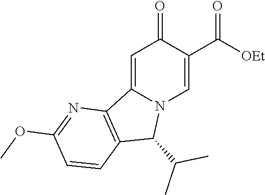 [SMILES: CCOC(=O)c1cn2c(cc1=O)-c1nc(OC)ccc1[C@H]2C(C)C] | ethyl (R)-5-isopropyl-2-methoxy-9-oxo-5,9- dihydropyrido[2,3-a]indolizine-8-carboxylate |

| Structure | Nomenclature |
|---|---|
| 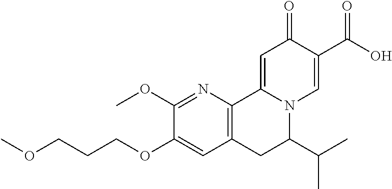 [SMILES: COCCCOc1cc2c(nc1OC)-c1cc(=O)c(C(=O)O)cn1C(C(C)C)C2] | 6-isopropyl-2-methoxy-3-(3-methoxypropoxy)- 10-oxo-5,10-dihydro-6H-pyrido[1,2- h][1,7]naphthyridine-9-carboxylic acid |
| 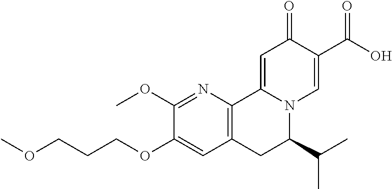 [SMILES: COCCCOc1cc2c(nc1OC)-c1cc(=O)c(C(=O)O)cn1[C@@H](C(C)C)C2] | (R)-6-isopropyl-2-methoxy-3-(3- methoxypropoxy)-10-oxo-5,10-dihydro-6H- pyrido[1,2-h][1,7]naphthyridine-9-carboxylic acid |
| 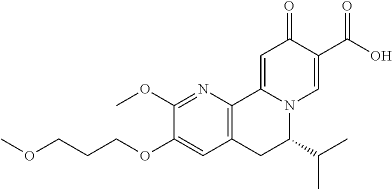 [SMILES: COCCCOc1cc2c(nc1OC)-c1cc(=O)c(C(=O)O)cn1[C@H](C(C)C)C2] | (S)-6-isopropyl-2-methoxy-3-(3- methoxypropoxy)-10-oxo-5,10-dihydro-6H- pyrido[1,2-h][1,7]naphthyridine-9-carboxylic acid |
| 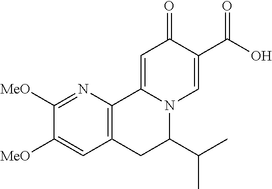 [SMILES: COc1cc2c(nc1OC)-c1cc(=O)c(C(=O)O)cn1C(C(C)C)C2] | 6-isopropyl-2,3-dimethoxy-10-oxo-5,10-dihydro- 6H-pyrido[1,2-h][1,7]naphthyridine-9-carboxylic acid |
| 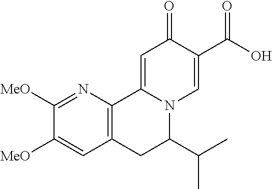 [SMILES: COc1cc2c(nc1OC)-c1cc(=O)c(C(=O)O)cn1C(C(C)C)C2] | 6-isopropyl-2,3-dimethoxy-10-oxo-5,10-dihydro- 6H-pyrido[1,2-h][1,7]naphthyridine-9-carboxylic acid (single enantiomer 1) |
| 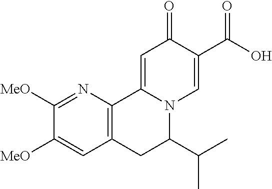 [SMILES: COc1cc2c(nc1OC)-c1cc(=O)c(C(=O)O)cn1C(C(C)C)C2] | 6-isopropyl-2,3-dimethoxy-10-oxo-5,10-dihydro- 6H-pyrido[1,2-h][1,7]naphthyridine-9-carboxylic acid (single enantiomer II) |
| 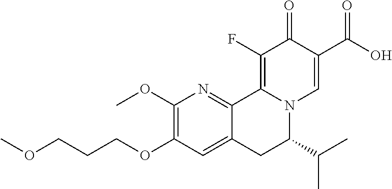 [SMILES: COCCCOc1cc2c(nc1OC)-c1c(F)c(=O)c(C(=O)O)cn1[C@H](C(C)C)C2] | (S)-11-fluoro-6-isopropyl-2-methoxy-3-(3- methoxypropoxy)-10-oxo-5,10-dihydro-6H- pyrido[1,2-h][1,7]naphthyridine-9-carboxylic acid |
| 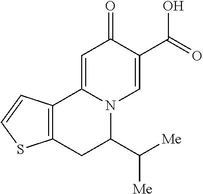 [SMILES: CC(C)C1Cc2sccc2-c2cc(=O)c(C(=O)O)cn21] | 5-isopropyl-9-oxo-4,9-dihydro-5H-thieno[3,2- a]quinolizine-8-carboxylic acid |
| 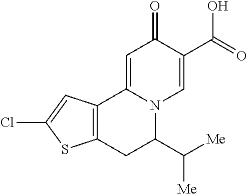 [SMILES: CC(C)C1Cc2sc(Cl)cc2-c2cc(=O)c(C(=O)O)cn21] | 2-chloro-5-isopropyl-9-oxo-4,9-dihydro-5H- thieno[3,2-a]quinolizine-8-carboxylic acid |
| 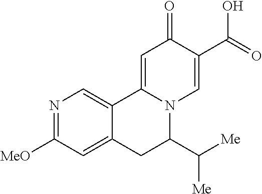 [SMILES: COc1cc2c(cn1)-c1cc(=O)c(C(=O)O)cn1C(C(C)C)C2] | 6-isopropyl-3-methoxy-10-oxo-5,10-dihydro-6H- pyrido[2,1-a][2,7]naphthyridine-9-carboxylic acid |
| 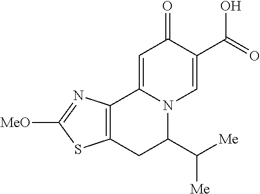 [SMILES: COc1nc2c(s1)CC(C(C)C)n1cc(C(=O)O)c(=O)cc1-2] | 5-isopropyl-2-methoxy-9-oxo-4,9-dihydro-5H- thiazolo[4,5-a]quinolizine-8-carboxylic acid |
| 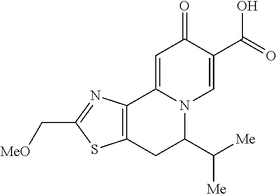 [SMILES: COCc1nc2c(s1)CC(C(C)C)n1cc(C(=O)O)c(=O)cc1-2] | 5-isopropyl-2-(methoxymethyl)-9-oxo-4,9- dihydro-5H-thiazolo[4,5-a]quinolizine-8- carboxylic acid |
| 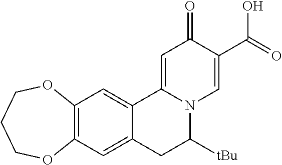 [SMILES: CC(C)(C)C1Cc2cc3c(cc2-c2cc(=O)c(C(=O)O)cn21)OCCCO3] | 6-(tert-butyl)-2-oxo-6,7,11,12-tetrahydro- 2H,10H-[1,4]dioxepino[2,3-g]pyrido[2,1- a]isoquinoline-3-carboxylic acid |
| 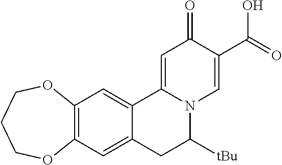 [SMILES: CC(C)(C)C1Cc2cc3c(cc2-c2cc(=O)c(C(=O)O)cn21)OCCCO3] | 6-(tert-butyl)-2-oxo-6,7,11,12-tetrahydro- 2H,10H-[1,4]dioxepino[2,3-g]pyrido[2,1- a]isoquinoline-3-carboxylic acid (single enantiomer I) |
| 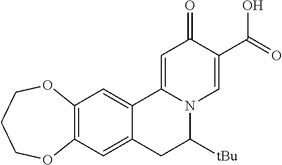 [SMILES: CC(C)(C)C1Cc2cc3c(cc2-c2cc(=O)c(C(=O)O)cn21)OCCCO3] | 6-(tert-butyl)-2-oxo-6,7,11,12-tetrahydro- 2H,10H-[1,4]dioxepino[2,3-g]pyrido[2,1- a]isoquinoline-3-carboxylic acid (single enantiomer II) |
| 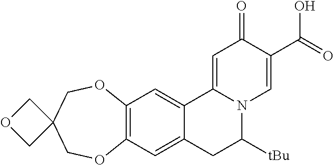 [SMILES: CC(C)(C)C1Cc2cc3c(cc2-c2cc(=O)c(C(=O)O)cn21)OCC1(COC1)CO3] | 6′-(tert-butyl)-2′-oxo-6′,7′-dihydro-2′H,10′H,12′H- spiro[oxetane-3,11′-[1,4]dioxepino[2,3- g]pyrido[2,1-a]isoquinoline]-3′-carboxylic acid |
| 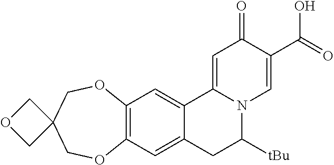 [SMILES: CC(C)(C)C1Cc2cc3c(cc2-c2cc(=O)c(C(=O)O)cn21)OCC1(COC1)CO3] | 6′-(tert-butyl)-2′-oxo-6′,7′-dihydro-2′H,10′H,12′H- spiro[oxetane-3,11′-[1,4]dioxepino[2,3- g]pyrido[2,1-a]isoquinoline]-3′-carboxylic acid (single enantiomer I) |
| 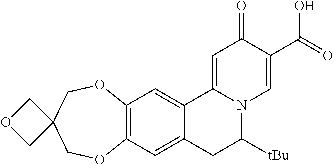 [SMILES: CC(C)(C)C1Cc2cc3c(cc2-c2cc(=O)c(C(=O)O)cn21)OCC1(COC1)CO3] | 6′-(tert-butyl)-2′-oxo-6′,7′-dihydro-2′H,10′H,12′H- spiro[oxetane-3,11′-[1,4]dioxepino[2,3- g]pyrido[2,1-a]isoquinoline]-3′-carboxylic acid (single enantiomer II) |
| 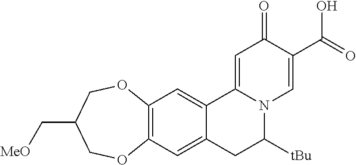 [SMILES: COCC1COc2cc3c(cc2OC1)-c1cc(=O)c(C(=O)O)cn1C(C(C)(C)C)C3] | 6-(tert-butyl)-11-(methoxymethyl)-2-oxo- 6,7,11,12-tetrahydro-2H,10H-[1,4]dioxepino[2,3- g]pyrido[2,1-a]isoquinoline-3-carboxylic acid |
| 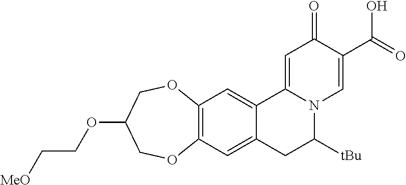 [SMILES: COCCOC1COc2cc3c(cc2OC1)-c1cc(=O)c(C(=O)O)cn1C(C(C)(C)C)C3] | 6-(tert-butyl)-11-(2-methoxyethoxy)-2-oxo- 6,7,11,12-tetrahydro-2H,10H-[1,4]dioxepino[2,3- g]pyrido[2,1-a]isoquinoline-3-carboxylic acid |
| 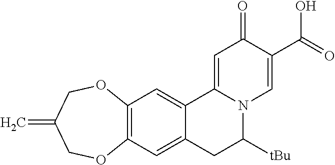 [SMILES: C=C1COc2cc3c(cc2OC1)-c1cc(=O)c(C(=O)O)cn1C(C(C)(C)C)C3] | 6-(tert-butyl)-11-methylene-2-oxo-6,7,11,12- tetrahydro-2H,10H-[1,4]dioxepino[2,3- g]pyrido[2,1-a]isoquinoline-3-carboxylic acid |
| 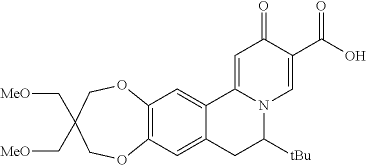 [SMILES: COCC1(COC)COc2cc3c(cc2OC1)-c1cc(=O)c(C(=O)O)cn1C(C(C)(C)C)C3] | 6-(tert-butyl)-11,11-bis(methoxymethyl)-2-oxo- 6,7,11,12-tetrahydro-2H,10H-[1,4]dioxepino[2,3- g]pyrido[2,1-a]isoquinoline-3-carboxylic acid |
| 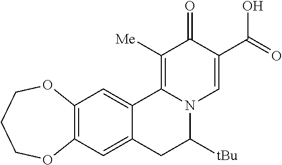 [SMILES: Cc1c2n(cc(C(=O)O)c1=O)C(C(C)(C)C)Cc1cc3c(cc1-2)OCCCO3] | 6-(tert-butyl)-1-methyl-2-oxo-6,7,11,12- tetrahydro-2H,10H-[1,4]dioxepino[2,3- g]pyrido[2,1-a]isoquinoline-3-carboxylic acid |
| 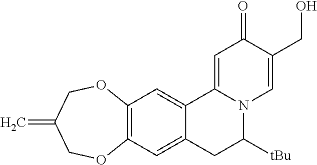 [SMILES: C=C1COc2cc3c(cc2OC1)-c1cc(=O)c(CO)cn1C(C(C)(C)C)C3] | 6-(tert-butyl)-3-(hydroxymethyl)-11-methylene- 6,7,11,12-tetrahydro-2H,10H-[1,4]dioxepino[2,3- g]pyrido[2,1-a]isoquinolin-2-one |
| 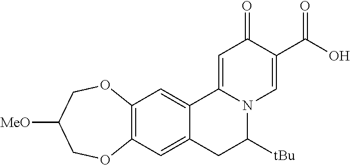 [SMILES: COC1COc2cc3c(cc2OC1)-c1cc(=O)c(C(=O)O)cn1C(C(C)(C)C)C3] | 6-(tert-butyl)-11-methoxy-2-oxo-6,7,11,12- tetrahydro-2H,10H-[1,4]dioxepino[2,3- g]pyrido[2,1-a]isoquinoline-3-carboxylic acid |
| 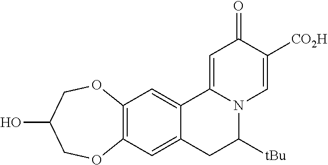 [SMILES: CC(C)(C)C1Cc2cc3c(cc2-c2cc(=O)c(C(=O)O)cn21)OCC(O)CO3] | 6-(tert-butyl)-11-hydroxy-2-oxo-6,7,11,12- tetrahydro-2H,10H-[1,4]dioxepino[2,3- g]pyrido[2,1-a]isoquinoline-3-carboxylic acid |
| 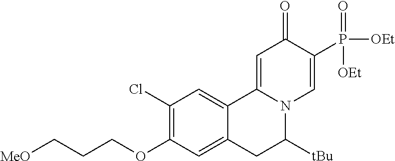 [SMILES: CCOP(=O)(OCC)c1cn2c(cc1=O)-c1cc(Cl)c(OCCCOC)cc1CC2C(C)(C)C] | diethyl (6-(tert-butyl)-10-chloro-9-(3- methoxypropoxy)-2-oxo-6,7-dihydro-2H- pyrido[2,1-a]isoquinolin-3-yl)phosphonate |
| 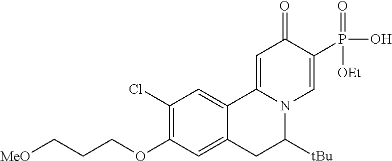 [SMILES: CCOP(=O)(O)c1cn2c(cc1=O)-c1cc(Cl)c(OCCCOC)cc1CC2C(C)(C)C] | ethyl hydrogen (6-(tert-butyl)-10-chloro-9-(3- methoxypropoxy)-2-oxo-6,7-dihydro-2H- pyrido[2,1-a]isoquinolin-3-yl)phosphonate |
| 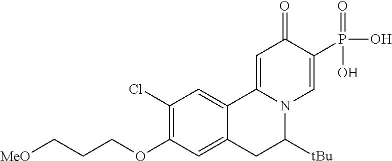 [SMILES: COCCCOc1cc2c(cc1Cl)-c1cc(=O)c(P(=O)(O)O)cn1C(C(C)(C)C)C2] | (6-(tert-butyl)-10-chloro-9-(3-methoxypropoxy)- 2-oxo-6,7-dihydro-2H-pyrido[2,1-a]isoquinolin- 3-yl)phosphonic acid |
| 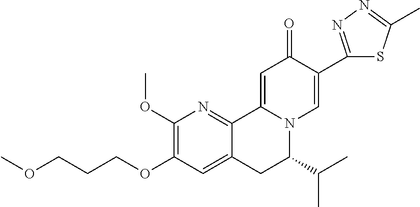 | (S)-6-isopropyl-2-methoxy-3-(3- methoxypropoxy)-9-(5-methyl-1,3,4-thiadiazol-2- yl)-5,6-dihydro-10H-pyrido[1,2- h][1,7]naphthyridin-10-one |
| 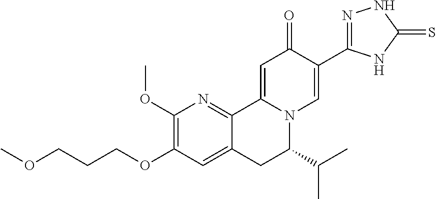 [SMILES: COCCCOc1cc2c(nc1OC)-c1cc(=O)c(-c3n[nH]c(=S)[nH]3)cn1[C@H](C(C)C)C2] | (S)-6-isopropyl-2-methoxy-3-(3- methoxypropoxy)-9-(5-thioxo-4,5-dihydro-1H- 1,2,4-triazol-3-yl)-5,6-dihydro-1H-pyrido[1,2- h][1,7]naphthyridin-10-one |
| 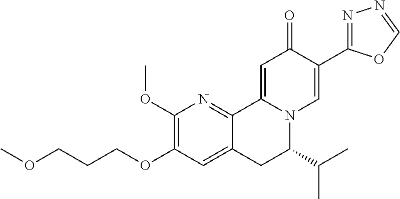 [SMILES: COCCCOc1cc2c(nc1OC)-c1cc(=O)c(-c3nnco3)cn1[C@H](C(C)C)C2] | (S)-6-isopropyl-2-methoxy-3-(3- methoxypropoxy)-9-(1,3,4-oxadiazol-2-yl)-5,6- dihydro-10H-pyrido[1,2-h][1,7]naphthyridin-10- one |
| 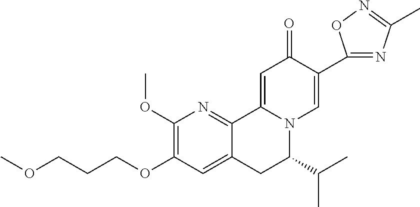 | (S)-6-isopropyl-2-methoxy-3-(3- methoxypropoxy)-9-(3-methyl-1,2,4-oxadiazol-5- yl)-5,6-dihydro-10H-pyrido[1,2- h][1,7]naphthyridin-10-one |
| 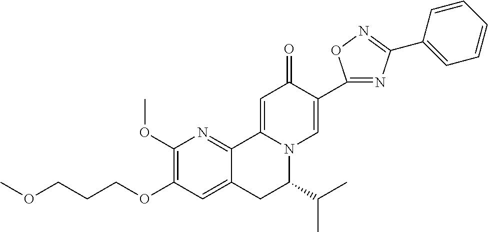 [SMILES: COCCCOc1cc2c(nc1OC)-c1cc(=O)c(-c3nc(-c4ccccc4)no3)cn1[C@H](C(C)C)C2] | (S)-6-isopropyl-2-methoxy-3-(3- methoxypropoxy)-9-(3-phenyl-1,2,4-oxadiazol-5- yl)-5,6-dihydro-10H-pyrido[1,2- h][1,7]naphthyridin-10-one |
| 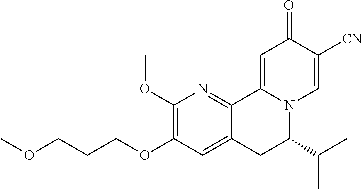 [SMILES: COCCCOc1cc2c(nc1OC)-c1cc(=O)c(C#N)cn1[C@H](C(C)C)C2] | (S)-6-isopropyl-2-methoxy-3-(3- methoxypropoxy)-10-oxo-5,10-dihydro-6H- pyrido[1,2-h][1,7]naphthyridine-9-carbonitrile |
| 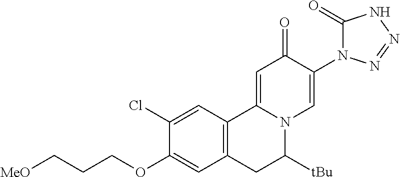 [SMILES: COCCCOc1cc2c(cc1Cl)-c1cc(=O)c(-n3nn[nH]c3=O)cn1C(C(C)(C)C)C2] | 6-(tert-butyl)-10-chloro-9-(3-methoxypropoxy)-3- (5-oxo-4,5-dihydro-1H-tetrazol-1-yl)-6,7- dihydro-2H-pyrido[2,1-a]isoquinolin-2-one |
| 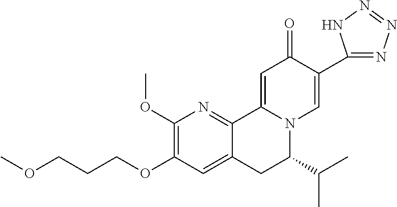 [SMILES: COCCCOc1cc2c(nc1OC)-c1cc(=O)c(-c3nnn[nH]3)cn1[C@H](C(C)C)C2] | (S)-6-isopropyl-2-methoxy-3-(3- methoxypropoxy)-9-(1H-tetrazol-5-yl)-5,6- dihydro-10H-pyrido[1,2-h][1,7]naphthyridin-10- one |
| 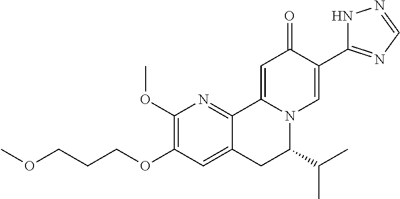 [SMILES: COCCCOc1cc2c(nc1OC)-c1cc(=O)c(-c3ncn[nH]3)cn1[C@H](C(C)C)C2] | (S)-6-isopropyl-2-methoxy-3-(3- methoxypropoxy)-9-(1H-1,2,4-triazol-5-yl)-5,6- dihydro-10H-pyrido[1,2-h][1,7]naphthyridin-10- one |
| 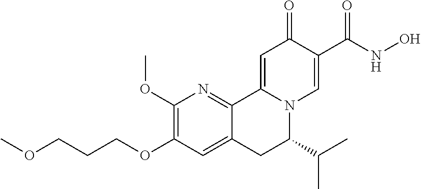 [SMILES: COCCCOc1cc2c(nc1OC)-c1cc(=O)c(C(=O)NO)cn1[C@H](C(C)C)C2] | (S)-N-hydroxy-6-isopropyl-2-methoxy-3-(3- methoxypropoxy)-10-oxo-5,10-dihydro-6H- pyrido[1,2-h][1,7]naphthyridine-9-carboxamide |
| 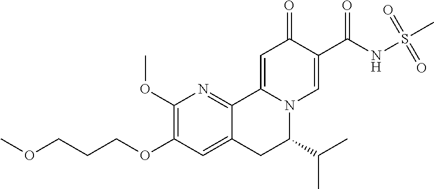 | (S)-6-isopropyl-2-methoxy-3-(3- methoxypropoxy)-N-(methylsulfonyl)-10-oxo- 5,10-dihydro-6H-pyrido[1,2- h][1,7]naphthyridine-9-carboxamide |
| 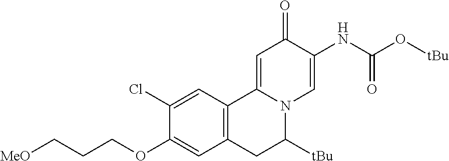 [SMILES: COCCCOc1cc2c(cc1Cl)-c1cc(=O)c(NC(=O)OC(C)(C)C)cn1C(C(C)(C)C)C2] | tert-butyl (6-(tert-butyl)-10-chloro-9-(3- methoxypropoxy)-2-oxo-6,7-dihydro-2H- pyrido[2,1-a]isoquinolin-3-yl)carbamate |
| 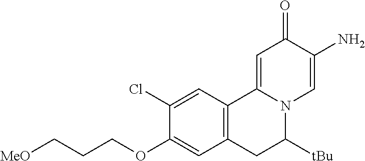 [SMILES: COCCCOc1cc2c(cc1Cl)-c1cc(=O)c(N)cn1C(C(C)(C)C)C2] | 3-amino-6-(tert-butyl)-10-chloro-9-(3- methoxypropoxy)-6,7-dihydro-2H-pyrido[2,1- a]isoquinolin-2-one |
| 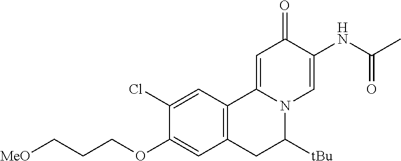 [SMILES: COCCCOc1cc2c(cc1Cl)-c1cc(=O)c(NC(C)=O)cn1C(C(C)(C)C)C2] | N-(6-(tert-butyl)-10-chloro-9-(3- methoxypropoxy)-2-oxo-6,7-dihydro-2H- pyrido[2,1-a]isoquinolin-3-yl)acetamide |
| 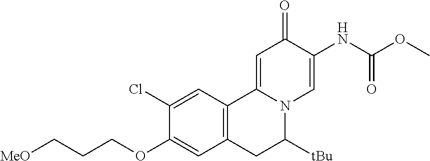 [SMILES: COCCCOc1cc2c(cc1Cl)-c1cc(=O)c(NC(=O)OC)cn1C(C(C)(C)C)C2] | methyl (6-(tert-butyl)-10-chloro-9-(3- methoxypropoxy)-2-oxo-6,7-dihydro-2H- pyrido[2,1-a]isoquinolin-3-yl)carbamate |
| 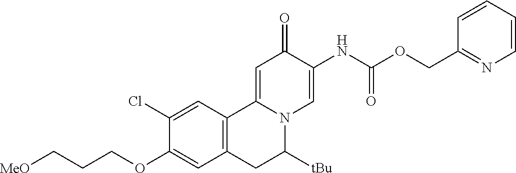 [SMILES: COCCCOc1cc2c(cc1Cl)-c1cc(=O)c(NC(=O)OCc3ccccn3)cn1C(C(C)(C)C)C2] | pyridin-2-ylmethyl (6-tert-butyl)-10-chloro-9-(3- methoxypropoxy)-2-oxo-6,7-dihydro-2H- pyrido[2,1-a]isoquinolin-3-yl)carbamate |
| 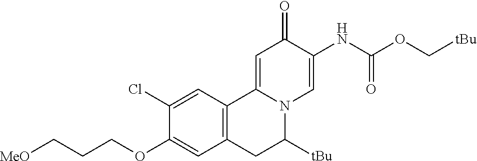 [SMILES: COCCCOc1cc2c(cc1Cl)-c1cc(=O)c(NC(=O)OCC(C)(C)C)cn1C(C(C)(C)C)C2] | neopentyl (6-(tert-butyl)-10-chloro-9-(3- methoxypropoxy)-2-oxo-6,7-dihydro-2H- pyrido[2,1-a]isoquinolin-3-yl)carbamate |
| 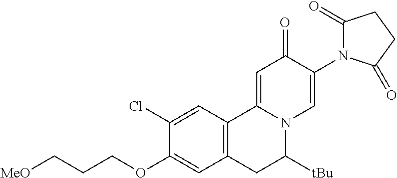 [SMILES: COCCCOc1cc2c(cc1Cl)-c1cc(=O)c(N3C(=O)CCC3=O)cn1C(C(C)(C)C)C2] | 1-(6-(tert-butyl)-10-chloro-9-(3- methoxypropxoy)-2-oxo-6,7-dihydro-2H- pyrido[2,1-a]isoquinolin-3-yl)pyrrolidine-2,5- dione |
| 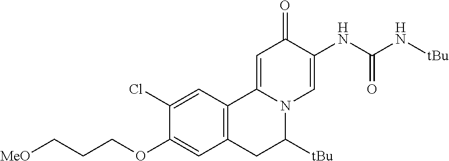 [SMILES: COCCCOc1cc2c(cc1Cl)-c1cc(=O)c(NC(=O)NC(C)(C)C)cn1C(C(C)(C)C)C2] | 1-(tert-butyl)-3-(6-(tert-butyl)-10-chloro-9-(3- methoxypropoxy)-2-oxo-6,7-dihydro-2H- pyrido[2,1-a]isoquinolin-3-yl)urea |
| 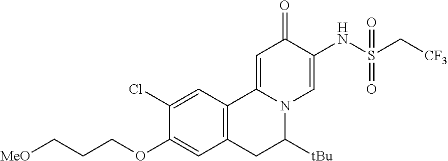 [SMILES: COCCCOc1cc2c(cc1Cl)-c1cc(=O)c(NS(=O)(=O)CC(F)(F)F)cn1C(C(C)(C)C)C2] | N-(6-(tert-butyl)-10-chloro-9-(3- methoxypropoxy)-2-oxo-6,7-dihydro-2H- pyrido[2,1-a]isoquinolin-3-yl)-2,2,2- trifluoroethane-1-sulfonamide |
| 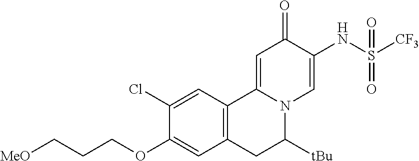 [SMILES: COCCCOc1cc2c(cc1Cl)-c1cc(=O)c(NS(=O)(=O)C(F)(F)F)cn1C(C(C)(C)C)C2] | N-(6-(tert-butyl)-10-chloro-9-(3- methoxypropoxy)-2-oxo-6,7-dihydro-2H- pyrido[2,1-a]isoquinolin-3-yl)-1,1,1- trifluoromethanesulfonamide |
| 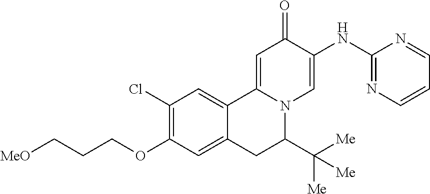 [SMILES: COCCCOc1cc2c(cc1Cl)-c1cc(=O)c(Nc3ncccn3)cn1C(C(C)(C)C)C2] | 6-(tert-butyl)-10-chloro-9-(3-methoxypropoxy)-3- (pyrimidin-2-ylamino)-6,7-dihydro-2H- pyrido[2,1-a]isoquinolin-2-one |
| 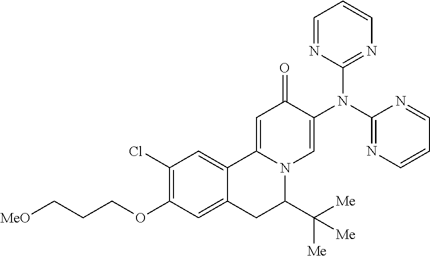 [SMILES: COCCCOc1cc2c(cc1Cl)-c1cc(=O)c(N(c3ncccn3)c3ncccn3)cn1C(C(C)(C)C)C2] | 6-(tert-butyl)-10-chloro-3-(di(pyrimidin-2- yl)amino)-9-(3-methoxypropoxy)-6,7-dihydro- 2H-pyrido[2,1-a]isoquinolin-2-one |
| 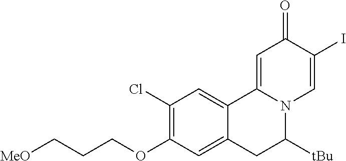 [SMILES: COCCCOc1cc2c(cc1Cl)-c1cc(=O)c(I)cn1C(C(C)(C)C)C2] | 6-(tert-butyl)-10-chloro-3-iodo-9-(3- methoxypropoxy)-6,7-dihydro-2H-pyrido[2,1- a]isoquinolin-2-one |
| 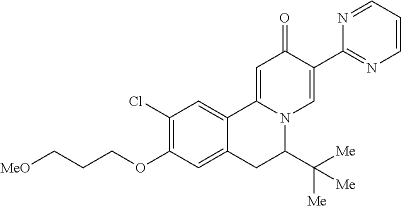 [SMILES: COCCCOc1cc2c(cc1Cl)-c1cc(=O)c(-c3ncccn3)cn1C(C(C)(C)C)C2] | 6-(tert-butyl)-10-chloro-9-(3-methoxypropoxy)-3- (pyrimidin-2-yl)-6,7-dihydro-2H-pyrido[2,1- a]isoquinolin-2-one |
| 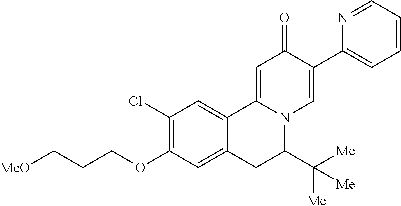 [SMILES: COCCCOc1cc2c(cc1Cl)-c1cc(=O)c(-c3ccccn3)cn1C(C(C)(C)C)C2] | 6-(tert-butyl)-10-chloro-9-(3-methoxypropoxy)-3- (pyridin-2-yl)-6,7-dihydro-2H-pyrido[2,1- a]isoquinolin-2-one |
| 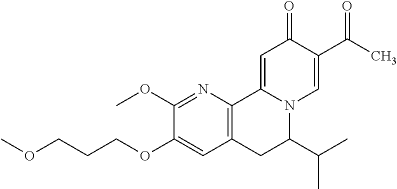 [SMILES: COCCCOc1cc2c(nc1OC)-c1cc(=O)c(C(C)=O)cn1C(C(C)C)C2] | 9-acetyl-6-isopropyl-2-methoxy-3-(3- methoxypropoxy)-5,6-dihydro-10H-pyrido[1,2- h][1,7]naphthyridin-1-one |
| 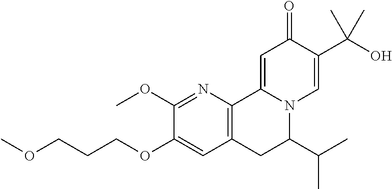 [SMILES: COCCCOc1cc2c(nc1OC)-c1cc(=O)c(C(C)(C)O)cn1C(C(C)C)C2] | 9-(2-hydroxypropan-2-yl)-6-isopropyl-2- methoxy-3-(3-methoxypropoxy)-5,6-dihydro- 10H-pyrido[1,2-h][1,7]naphthyridin-10-one |
| 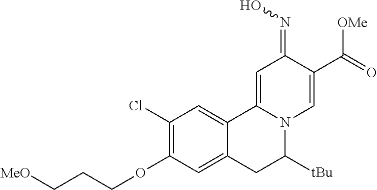 [SMILES: COCCCOc1cc2c(cc1Cl)-c1cc(=NO)c(C(=O)OC)cn1C(C(C)(C)C)C2] | methyl 6-(tert-butyl)-10-chloro-2- (hydroxyimino)-9-(3-methoxypropoxy)-6,7- dihydro-2H-pyrido[2,1-a]isoquinoline-3- carboxylate |
| 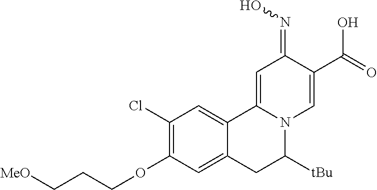 [SMILES: COCCCOc1cc2c(cc1Cl)-c1cc(=NO)c(C(=O)O)cn1C(C(C)(C)C)C2] | 6-(tert-butyl)-10-chloro-2-(hydroxyimino)-9-(3- methoxypropoxy)-6,7-dihydro-2H-pyrido[2,1- a]isoquinoline-3-carboxylic acid |
| 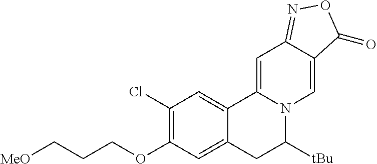 [SMILES: COCCCOc1cc2c(cc1Cl)-c1cc3noc(=O)c-3cn1C(C(C)(C)C)C2] | 6-(tert-butyl)-2-chloro-3-(3-methoxypropoxy)- 5,6-dihydro-9H-isoxazolo[3′,4′:4,5]pyrido[2,1- a]isoquinolin-9-one |
| 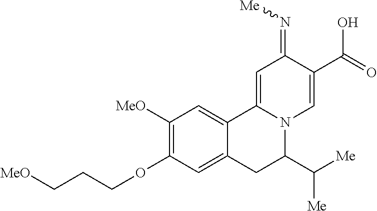 | 6-isopropyl-10-methoxy-9-(3-methoxypropoxy)- 2-(methylimino)-6,7-dihydro-2H-pyrido[2,1- a]isoquinoline-3-carboxylic acid |
| 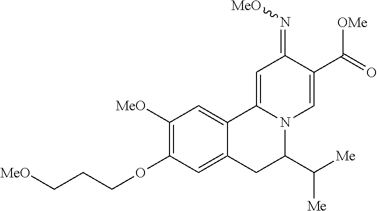 [SMILES: COCCCOc1cc2c(cc1OC)-c1cc(=NOC)c(C(=O)OC)cn1C(C(C)C)C2] | methyl 6-isopropyl-10-methoxy-2- (methoxyimino)-9-(3-methoxypropoxy)-6,7- dihydro-2H-pyrido[2,1-a]isoquinoline-3- carboxylate |
| 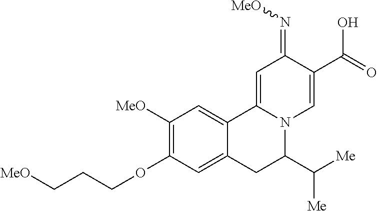 [SMILES: COCCCOc1cc2c(cc1OC)-c1cc(=NOC)c(C(=O)O)cn1C(C(C)C)C2] | 6-isopropyl-10-methoxy-2-(methoxyimino)-9-(3- methoxypropoxy)-6,7-dihydro-2H-pyrido[2,1- a]isoquinoline-3-carboxylic acid |
| 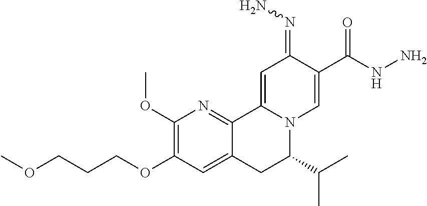 [SMILES: COCCCOc1cc2c(nc1OC)-c1cc(=NN)c(C(=O)NN)cn1[C@H](C(C)C)C2] | (S)-10-hydrazineylidene-6-isopropyl-2-methoxy- 3-(3-methoxypropoxy)-5,10-dihydro-6H- pyrido[1,2-h][1,7]naphthyridine-9-carbohydrazide |
| 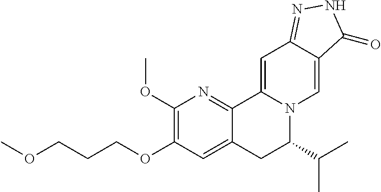 [SMILES: COCCCOc1cc2c(nc1OC)-c1cc3n[nH]c(=O)c-3cn1[C@H](C(C)C)C2] | (S)-6-isopropyl-2-methoxy-3-(3- methoxypropoxy)-5,10- dihydropyrazolo[3′,4′:4,5]pyrido[1,2- h][1,7]naphthyridin-9(6H)-one |
| 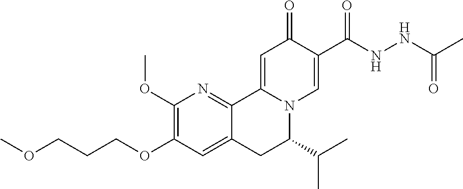 [SMILES: COCCCOc1cc2c(nc1OC)-c1cc(=O)c(C(=O)NNC(C)=O)cn1[C@H](C(C)C)C2] | (S)-N′-acetyl-6-isopropyl-2-methoxy-3-(3- methoxypropoxy)-10-oxo-5,10-dihydro-6H- pyrido[1,2-h][1,7]naphthyridine-9-carbohydrazide |
| 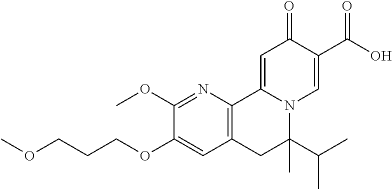 [SMILES: COCCCOc1cc2c(nc1OC)-c1cc(=O)c(C(=O)O)cn1C(C)(C(C)C)C2] | 6-isopropyl-2-methoxy-3-(3-methoxypropoxy)-6- methyl-10-oxo-5,10-dihydro-6H-pyrido[1,2- h][1,7]naphthyridine-9-carboxylic acid |
| 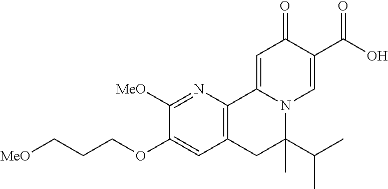 [SMILES: COCCCOc1cc2c(nc1OC)-c1cc(=O)c(C(=O)O)cn1C(C)(C(C)C)C2] | 6-isopropyl-2-methoxy-3-(3-methoxypropoxy)-6- methyl-10-oxo-4,10-dihydro-6H-pyrido[1,2- h][1,7]naphthyridine-9-carboxylic acid (single enantiomer I) |
| 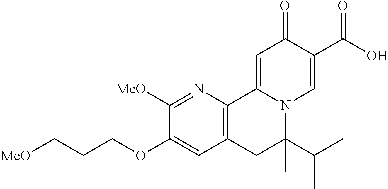 [SMILES: COCCCOc1cc2c(nc1OC)-c1cc(=O)c(C(=O)O)cn1C(C)(C(C)C)C2] | 6-isopropyl-2-methoxy-3-(3-methoxypropoxy)-6- methyl-10-oxo-5,10-dihydro-6H-pyrido[1,2- h][1,7]naphthyridine-9-carboxylic acid (single enantiomer II) |
| 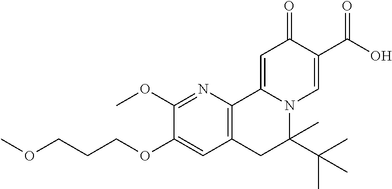 [SMILES: COCCCOc1cc2c(nc1OC)-c1cc(=O)c(C(=O)O)cn1C(C)(C(C)(C)C)C2] | 6-(tert-butyl)-2-methoxy-3-(3-methoxypropoxy)- 6-methyl-10-oxo-5,10-dihydro-6H-pyrido[1,2- h][1,7]naphthyridine-9-carboxylic acid |
| 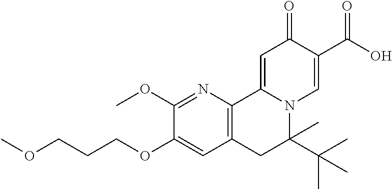 [SMILES: COCCCOc1cc2c(nc1OC)-c1cc(=O)c(C(=O)O)cn1C(C)(C(C)(C)C)C2] | 6-(tert-butyl)-2-methoxy-3-(3-methoxypropoxy)- 6-methyl-10-oxo-5,10-dihydro-6H-pyrido[1,2- h][1,7]naphthyridine-9-carboxylic acid (single enantiomer I) |
| 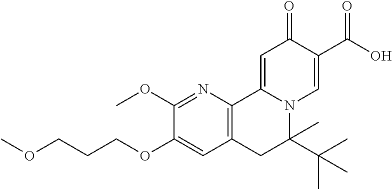 [SMILES: COCCCOc1cc2c(nc1OC)-c1cc(=O)c(C(=O)O)cn1C(C)(C(C)(C)C)C2] | 6-(tert-butyl)-2-methoxy-3-(3-methoxypropoxy)- 6-methyl-10-oxo-5,10-dihydro-6H-pyrido[1,2- h][1,7]naphthyridine-9-carboxylic acid (single enantiomer II) |
| 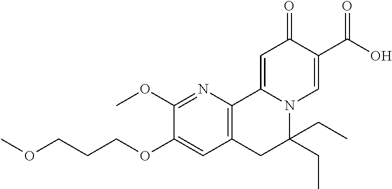 [SMILES: CCC1(CC)Cc2cc(OCCCOC)c(OC)nc2-c2cc(=O)c(C(=O)O)cn21] | 6,6-diethyl-2-methoxy-3-(3-methoxypropoxy)- 10-oxo-5,10-dihydro-6H-pyrido[1,2- h][1,7]naphthyridine-9-carboxylic acid |
| 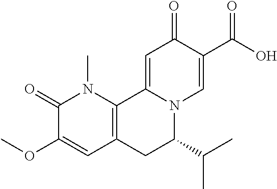 [SMILES: COc1cc2c(n(C)c1=O)-c1cc(=O)c(C(=O)O)cn1[C@H](C(C)C)C2] | (S)-6-isopropyl-3-methoxy-1-methyl-2,10-dioxo- 2,5,6,10-tetrahydro-1H-pyrido[1,2- h][1,7]naphthyridine-9-carboxylic acid |
| 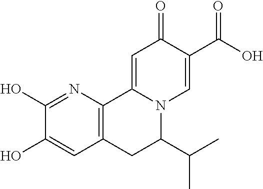 [SMILES: CC(C)C1Cc2cc(O)c(O)nc2-c2cc(=O)c(C(=O)O)cn21] | 2,3-dihydroxy-6-isopropyl-10-oxo-5,10-dihydro- 6H-pyrido[1,2-h][1,7]naphthyridne-9-carboxylic acid |
| 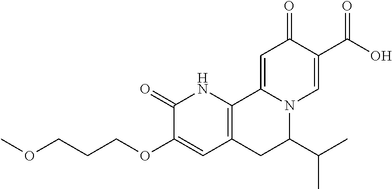 [SMILES: COCCCOc1cc2c([nH]c1=O)-c1cc(=O)c(C(=O)O)cn1C(C(C)C)C2] | 6-isopropyl-3-(3-methoxypropoxy)-2,10-dioxo- 2,5,6,10-tetrahydro-1H-pyrido[1,2- h][1,7]naphthyridine-9-carboxylic acid (single enantiomer I) |
| 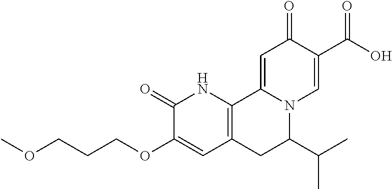 [SMILES: COCCCOc1cc2c([nH]c1=O)-c1cc(=O)c(C(=O)O)cn1C(C(C)C)C2] | 6-isopropyl-3-(3-methoxypropoxy)-2,10-dioxo- 2,5,6,10-tetrahydro-1H-pyrido[1,2- h][1,7]naphthyridine-9-carboxylic acid (single enantiomer II) |
| 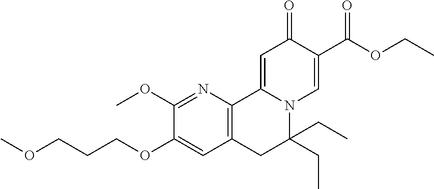 [SMILES: CCOC(=O)c1cn2c(cc1=O)-c1nc(OC)c(OCCCOC)cc1CC2(CC)CC] | ethyl 6,6-diethyl-2-methoxy-3-(3- methoxypropoxy)-10-oxo-5,10-dihydro-6H- pyrido[1,2-h][1,7]naphthyridine-9-carboxylate |
| 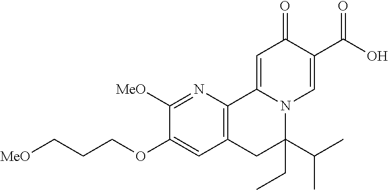 [SMILES: CCC1(C(C)C)Cc2cc(OCCCOC)c(OC)nc2-c2cc(=O)c(C(=O)O)cn21] | 6-ethyl-6-isopropyl-2-methoxy-3-(3- methoxypropoxy)-10-oxo-5,10-dihydro-6H- pyrido[1,2-h][1,7]naphthyridine-9-carboxylic acid |
| 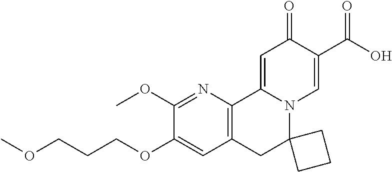 [SMILES: COCCCOc1cc2c(nc1OC)-c1cc(=O)c(C(=O)O)cn1C1(CCC1)C2] | 2′-methoxy-3′-(3-methoxypropoxy)-10′-oxo- 5′,10′-dihydrospiro[cyclobutane-1,6′-pyrido[1,2- h][1,7]naphthyridine]-9′-carboxylic acid |

### Immunostimulators

The term “immunostimulator” includes compounds that are capable of modulating an immune response (e.g., stimulate an immune response (e.g., an adjuvant)). The term immunostimulators includes polyinosinic:polycytidylic acid (poly I:C) and interferons.

The term immunostimulators includes agonists of stimulator of IFN genes (STING) and interleukins. The term also includes HIBsAg release inhibitors, TLR-7 agonists (GS-9620, RG-7795), T-cell stimulators (GS-4774), RIG-1 inhibitors (SB-9200), and SMAC-mimetics (Birinapant). The term immunostimulators also includes anti-PD-1 antibodies, and fragments thereof.

siRNA Conjugates

Conjugates useful in the practice of the methods provided herein are described in the following patent documents: U.S. Pat. No. 8,828,956; WO 2016/077321; WO 2017/177326; and WO 2018/191278. Each of the above patent documents is specifically incorporated by reference in its entirety.

In certain embodiments, the siRNA of the conjugate is selected from the following siRNA sequences. It should be understood that the following references to siRNA Number and SEQ ID NO are defined with respect to references to siRNA conjugate molecules, e.g., GaNAc-siRNA conjugates.

Chemically Modified HBV siRNA duplexes

| | Sense | | Antisense | |
|---|---|---|---|---|
| siRNA | strand | | strand SEQ | |
| Number | SEQ ID NO | Sense strand 5’-3’ | ID NO | Antisense strand 5’-3’ |
| 1 | SEQ ID | csgsugugCaCUUcgcuucaccu | SEQ ID | asGsgugAaGCgaagUgCacacgsgsuUU |
| | NO: 1 | | NO: 2 | |
| 2 | SEQ ID | usgsCaCUUcgcuucaccu | SEQ ID | asGsgugAaGCgaagUgCacascsgU |
| | NO: 3 | | NO: 4 | |
| 3 | SEQ ID | usgscaCUUcgcuucaccu | SEQ ID | asGsgugaagcgaagUgCacascsgU |
| | NO: 5 | | NO: 6 | |
| 4 | SEQ ID | usgscaCUUCgcuucaccu | SEQ ID | asGsgugAagcgaagUgCacascsgU |
| | NO: 7 | | NO: 8 | |
| 5 | SEQ ID | CscsGuGuGcACUucGcuuCacc | SEQ ID | gsGsUgAaGcgAaguGcAcAcGgsusc |
| | NO: 9 | | NO: 10 | |
| 6 | SEQ ID | cscsguguGcACUucgcuucacc | SEQ ID | gsGsugaAgCGaaguGcAcacggsusc |
| | NO: 11 | | NO: 12 | |
| 7 | SEQ ID | cscsguGuGcAcUucgcuucacc | SEQ ID | gsGsugaAgCGaaguGcAcacggsusc |
| | NO: 13 | | NO: 14 | |
| 8 | SEQ ID | cscsguguGcACUucgcuuCacc | SEQ ID | gsGsugaAgCgaaguGcAcacGgsusc |
| | NO: 15 | | NO: 16 | |
| 9 | SEQ ID | cscsgugugcACUucgcuucacc | SEQ ID | gsGsugaagcgaaguGcAcacggsusc |
| | NO: 17 | | NO: 18 | |
| 10 | SEQ ID | cscsguguGcacuucgcuucacc | SEQ ID | gsgsugaAgCGaagugcacacggsusc |
| | NO: 19 | | NO: 20 | |
| 11 | SEQ ID | CscsGuGuGcACUucGcuuCacc | SEQ ID | gsGsUgAaGcgAaguGcAcAcGgsuscUU |
| | NO: 21 | | NO: 22 | |
| 12 | SEQ ID | cscsguguGcACUucgcuucacc | SEQ ID | gsGsugaAgCGaaguGcAcacggsuscUU |
| | NO: 23 | | NO: 24 | |
| 13 | SEQ ID | cscsguGuGcAcUucgcuucacc | SEQ ID | gsGsugaAgCGaaguGcAcacggsuscUU |
| | NO: 25 | | NO: 26 | |
| 14 | SEQ ID | cscsguguGcACUucgcuuCacc | SEQ ID | gsGsugaAgCgaaguGcAcacGgsuscUU |
| | NO: 27 | | NO: 28 | |
| 15 | SEQ ID | GsusGcACUucGcuuCacc | SEQ ID | gsGsUgAaGcgAaguGcAcAcsGsgU |
| | NO: 29 | | NO: 30 | |
| 16 | SEQ ID | GsusGcACUucGcuuCacc | SEQ ID | gsGsUgAaGcgAaguGcAcAcsGsg |
| | NO: 31 | | NO: 32 | |
| 17 | SEQ ID | GsusGcACUucGcuuCacc | SEQ ID | gsGsUgAaGcgAaguGcAcsAscsGsg |
| | NO: 33 | | NO: 34 | |
| 18 | SEQ ID | CscsGuGuGcACUucGcuuCaca | SEQ ID | usGsUgAaGcgAaguGcAcAcGgsusc |
| | NO: 35 | | NO: 36 | |
| 19 | SEQ ID | CscsGuGuGcACUucGcuuCaca | SEQ ID | usGsUgAaGcgAaguGcAcAcGgsuscU |
| | NO: 37 | | NO: 38 | U |
| 20 | SEQ ID | cscsguguGcACUucgcuucaca | SEQ ID | usGsugaAgCGaaguGcAcacggsuscUU |
| | NO: 39 | | NO: 40 | |
| 21 | SEQ ID | cscsguGuGcAcUucgcuucaca | SEQ ID | usGsugaAgCGaaguGcAcacggsuscUU |
| | NO: 41 | | NO: 42 | |
| 22 | SEQ ID | cscsguguGcACUucgcuuCaca | SEQ ID | usGsugaAgCgaaguGcAcacGgsuscUU |
| | NO: 43 | | NO: 44 | |
| 23 | SEQ ID | cscsgugugcACUucgcuucaca | SEQ ID | usGsugaagcgaaguGcAcacggsuscUU |
| | NO: 45 | | NO: 46 | |
| 24 | SEQ ID | gsusGcACUucgcuucaca | SEQ ID | usGsugaAgCGaaguGcAcacsgsgU |
| | NO: 47 | | NO: 48 | |
| 25 | SEQ ID | gsusgcACUucgcuucaca | SEQ ID | usGsugaagcgaaguGcAcacsgsgU |
| | NO: 49 | | NO: 50 | |
| 26 | SEQ ID | gsusGcaCUucgcuucaca | SEQ ID | usGsugaagcgaaguGcAcacsgsgU |
| | NO: 51 | | NO: 52 | |
| 27 | SEQ ID | GsusGcACUucGcuuCaca | SEQ ID | usGsUgAaGcgAaguGcAcAcsGsg |
| | NO: 53 | | NO: 54 | |
| 28 | SEQ ID | uscsgcuuCaCCUcugcacgucg | SEQ ID | csGsacgUgCAgaggUgAagcgasasgUU |
| | NO: 55 | | NO: 56 | |
| 29 | SEQ ID | uscsgcuuCaCCUcugcacguca | SEQ ID | usGsacgUgCAgaggUgAagcgasasgUU |
| | NO: 57 | | NO: 58 | |
| 30 | SEQ ID | uscsgcUuCaCcUcugcacguca | SEQ ID | usGsacgUgCAgaggUgAagcgasasgUU |
| | NO: 59 | | NO: 60 | |
| 31 | SEQ ID | ususCaCCUcugcacguca | SEQ ID | usGsacgUgCAgaggUgAagcsgsaU |
| | NO: 61 | | NO: 62 | |
| 32 | SEQ ID | ususcaCCUcugcacguca | SEQ ID | usGsacgugcagaggUgAagcsgsaU |
| | NO: 63 | | NO: 64 | |
| 33 | SEQ ID | ususCaCCUcugcacguca | SEQ ID | usGsacgUgcagaggUgAagcsgsaU |
| | NO: 65 | | NO: 66 | |
| 34 | SEQ ID | ususuaCuAgUGCcaUuuguuca | SEQ ID | usGsAaCaAauGgcaCuAgUaAascsu |
| | NO: 67 | | NO: 68 | |
| 35 | SEQ ID | ususuaCuAgUGCcaUuuguuca | SEQ ID | usGsAaCaAauGgcaCuAgUaAascsuUU |
| | NO: 69 | | NO: 70 | |
| 36 | SEQ ID | ususuacuAgUGCcauuuguuca | SEQ ID | usGsaacAaAUggcaCuAguaaascsuUU |
| | NO: 71 | | NO: 72 | |
| 37 | SEQ ID | ususuaCuAgUgCcauuuguuca | SEQ ID | usGsaacAaAUggcaCuAguaaascsuUU |
| | NO: 73 | | NO: 74 | |

2’-O-Methyl nucleotides = lower case; 2’-Fluoro nucleotides = UPPER_CASE; Phosphorothioate linker = s; Unmodified = UPPER CASE

In certain embodiments, the conjugate is a conjugate of the following formula:

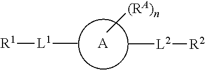

wherein the following definitions apply:

R¹ a is targeting ligand;

L¹ is absent or a linking group;

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen or a protecting group; and

n is 0, 1, 2, 3, 4, 5, 6, 7, 8, 9, or 10;

or a salt thereof.

In certain embodiments,

R¹ a is targeting ligand;

L¹ is absent or a linking group;

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B} and C₁₋₈ alkyl that is optionally substituted with one or more groups independently selected from halo, hydroxy, and C₁₋₃ alkoxy;

R^{B} is hydrogen or a protecting group; and

n is 0, 1, 2, 3, 4, 5, 6, 7, 8, 9, or 10.

In certain embodiments, the conjugate is a conjugate of the formula:

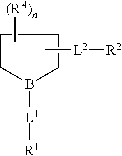

wherein:

B is —N— or —CH—;

L² is C₁₋₄ alkylene-O— that is optionally substituted with hydroxyl or halo; and

n is 0, 1, 2, 3, 4, 5, 6, or 7.

In certain embodiments, the conjugate is selected from the group consisting of:

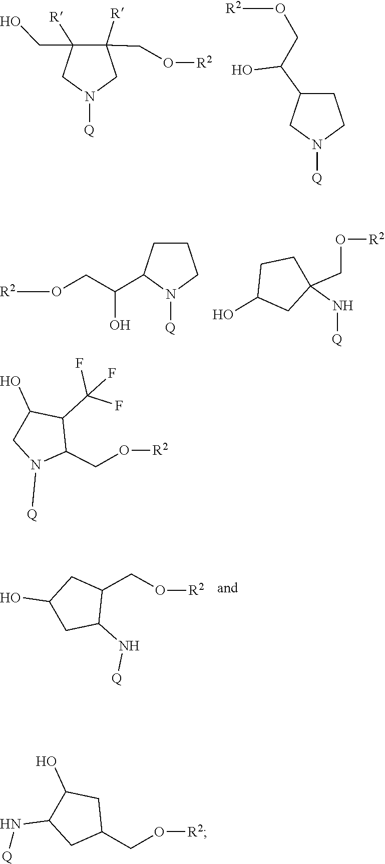

wherein Q is -L¹-R¹; and

R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

In certain embodiments, the conjugate is selected from the group consisting of:

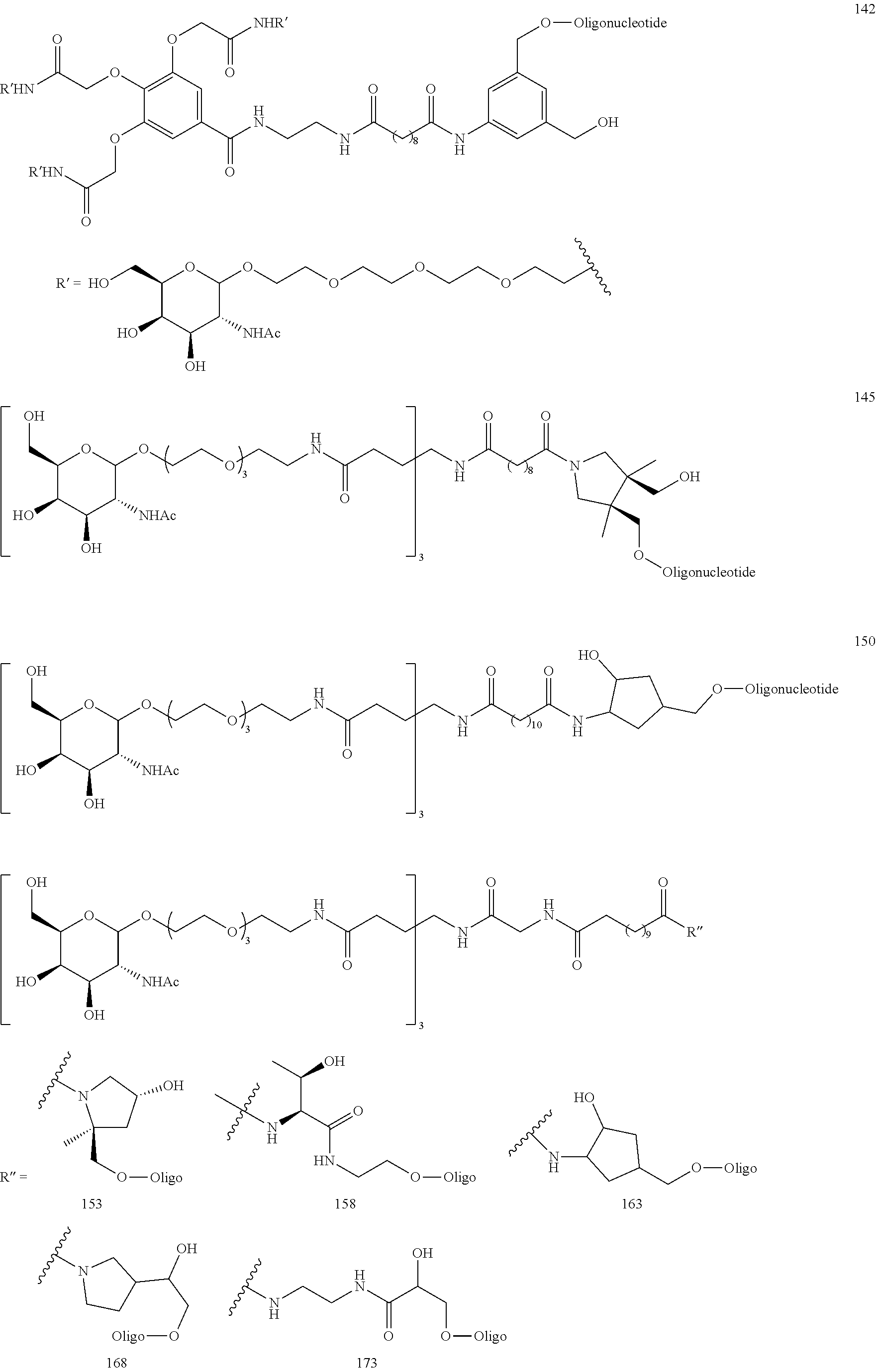

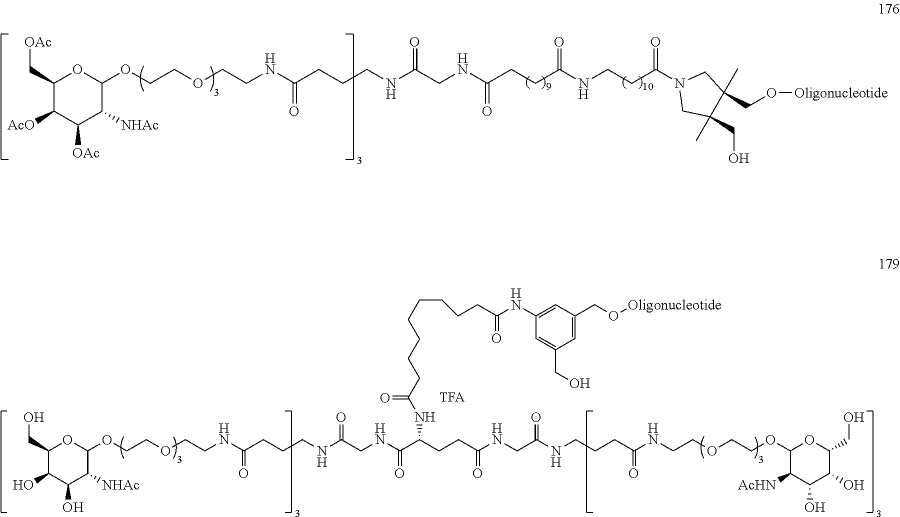

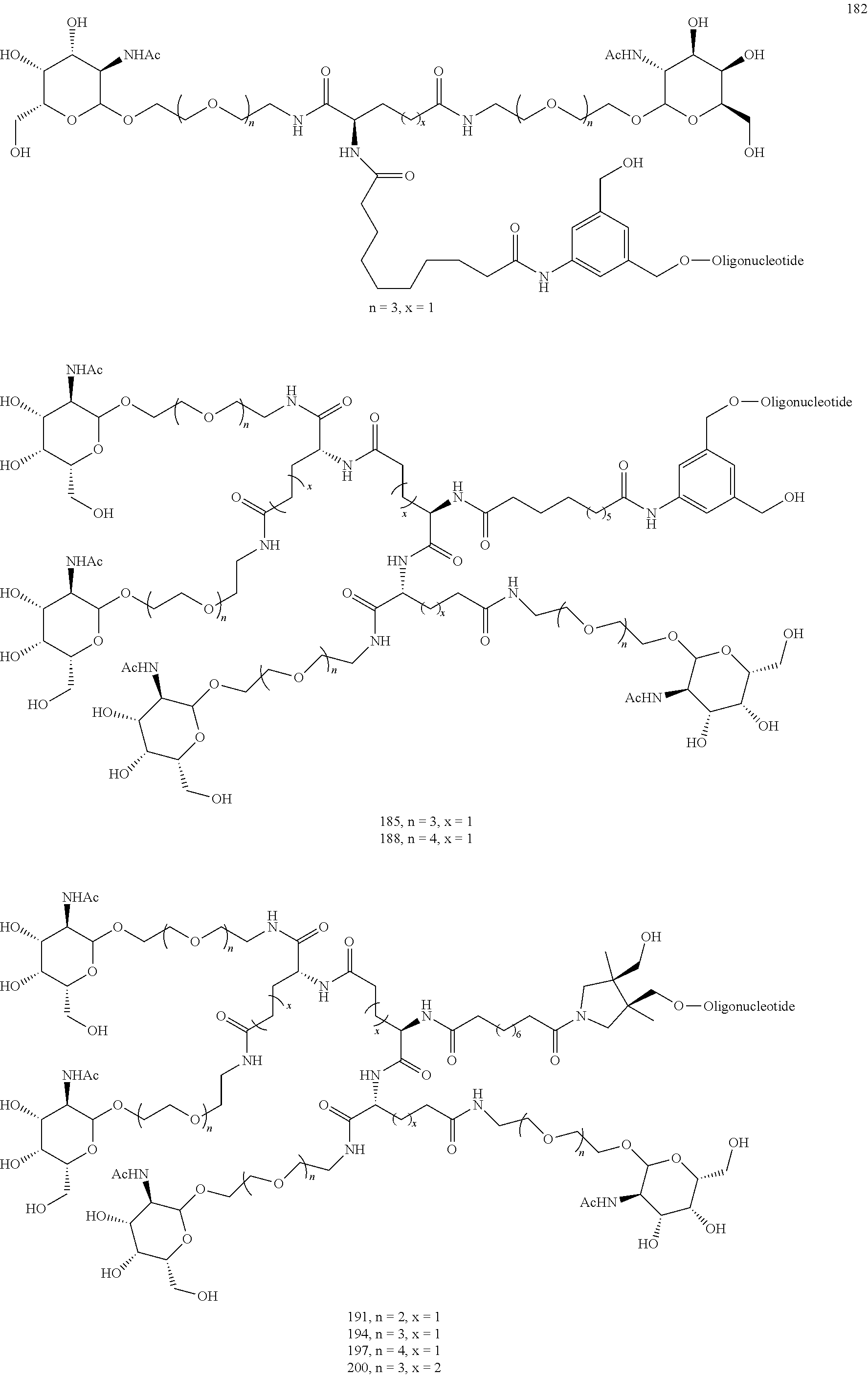

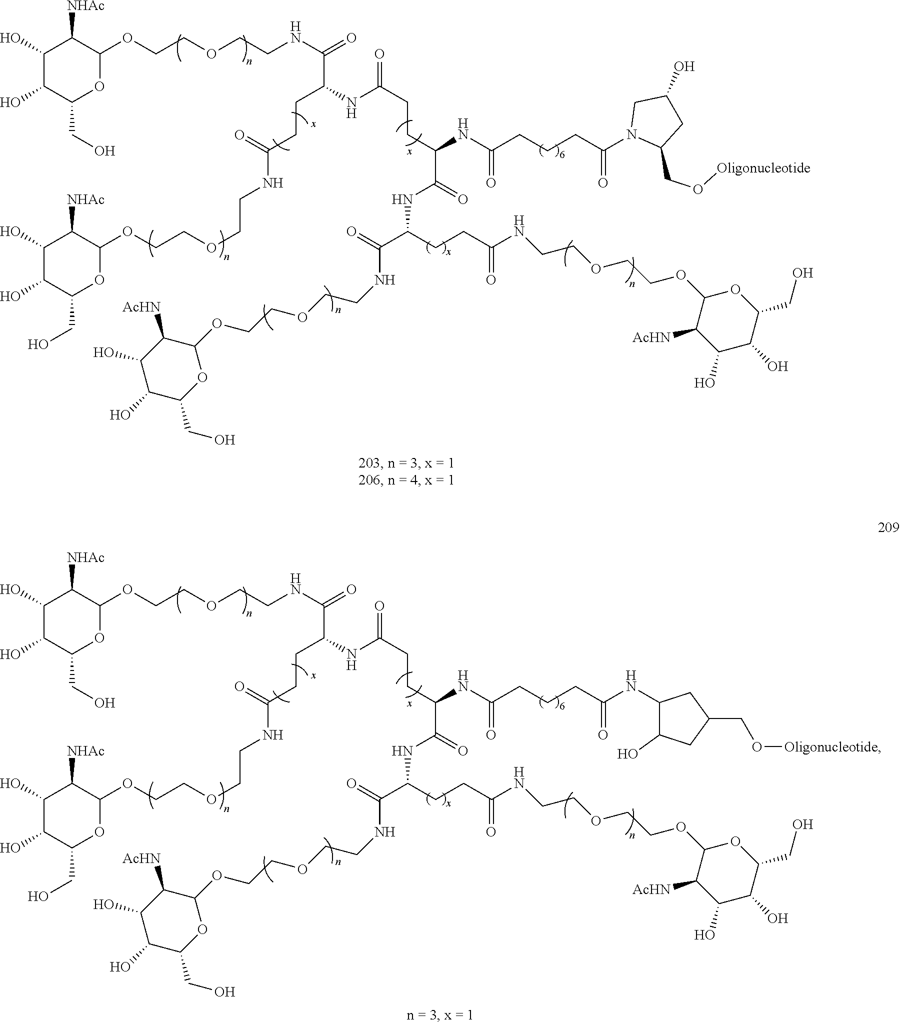

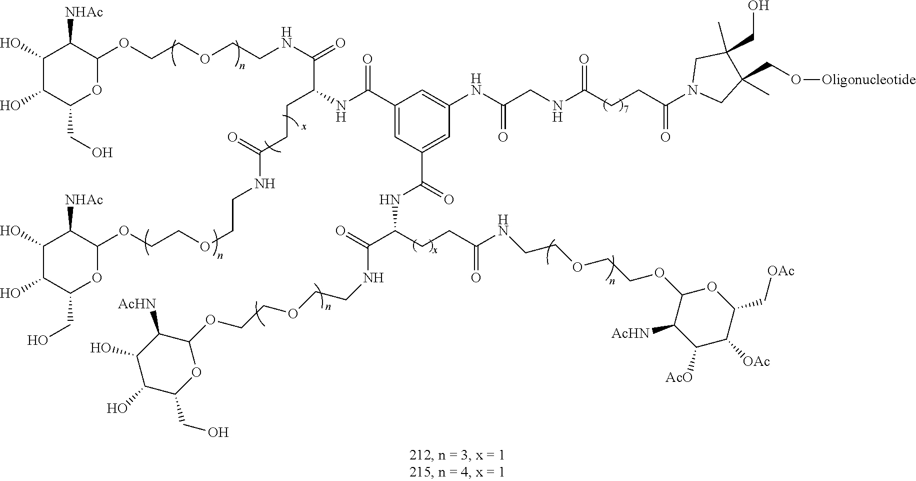

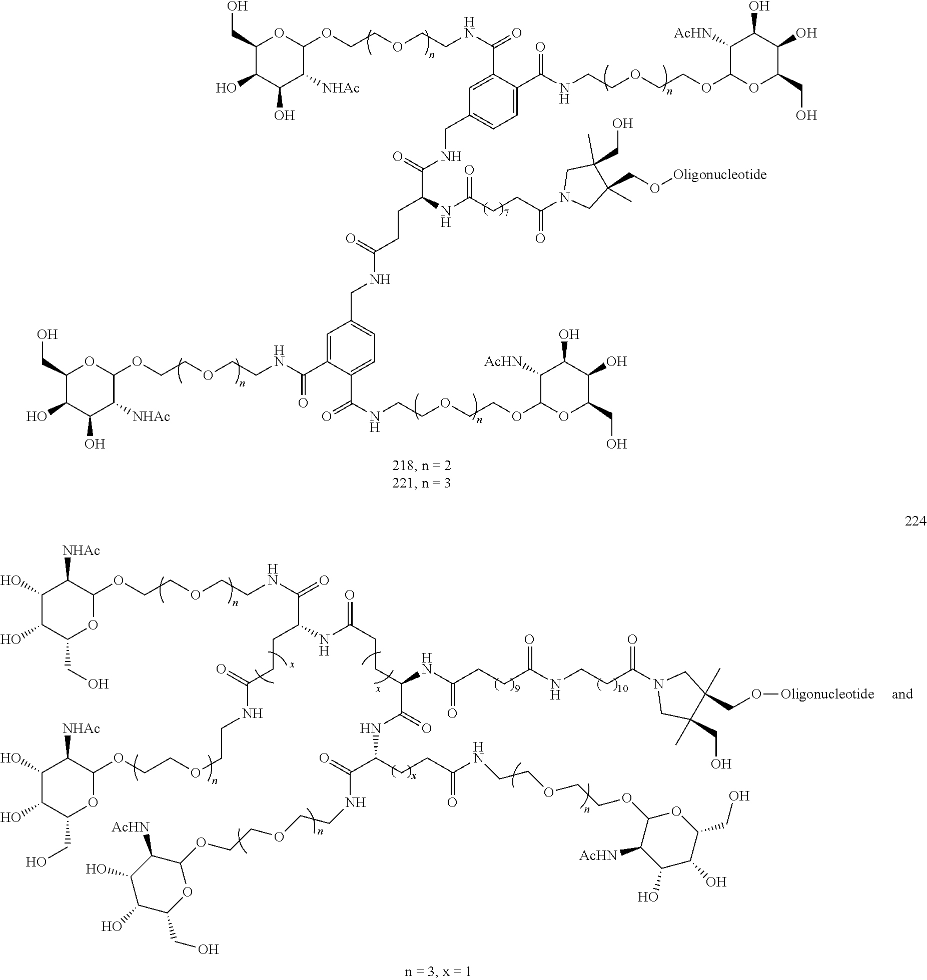

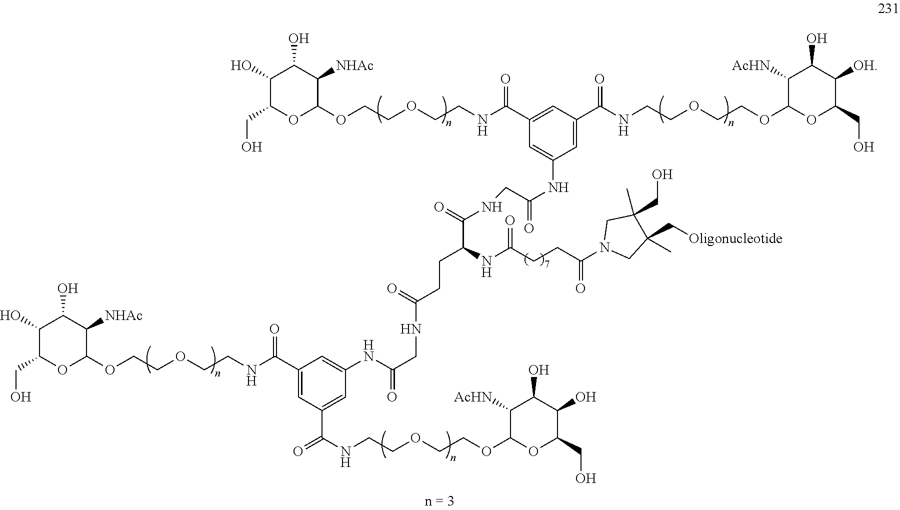

In certain embodiments, Ring A is selected from the group consisting of:

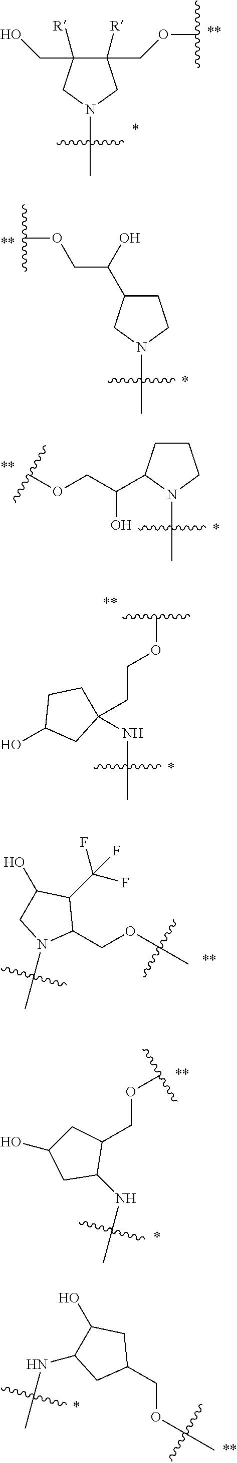

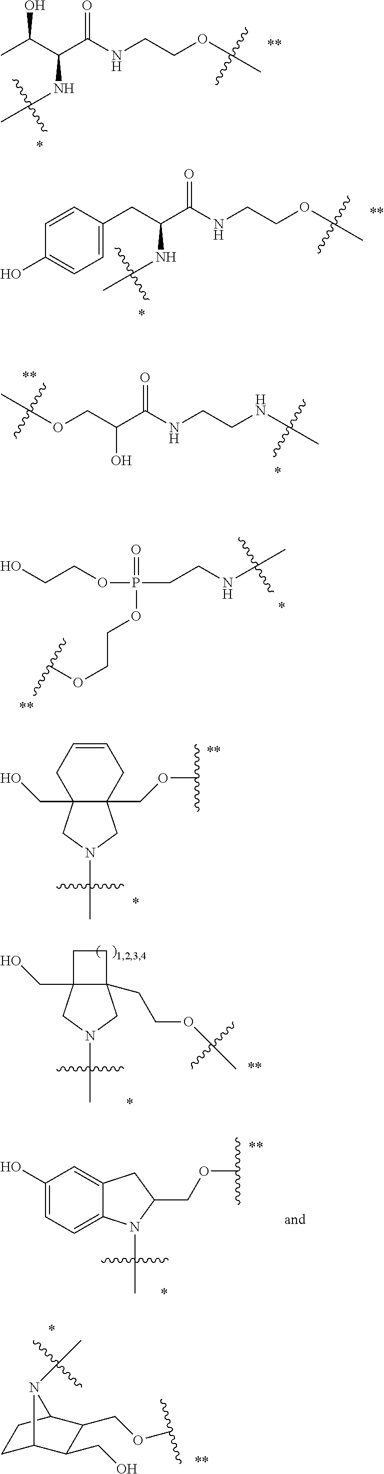

wherein:

each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;

the valence marked with * is attached to L¹ or is attached to R¹ if L¹ is absent; and

the valence marked with ** is attached to L² or is attached to R² if L² is absent.

In certain embodiments, the targeting ligand R¹ comprises 2-4 saccharides.

In certain embodiments, R¹ has the following formula:

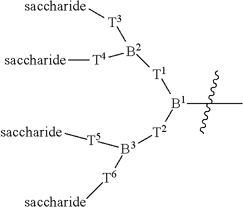

wherein:

B¹ is a trivalent group comprising about 1 to about 20 atoms and is covalently bonded to L¹, T¹, and T².

B² is a trivalent group comprising about 1 to about 20 atoms and is covalently bonded to T¹, T³, and T⁴;

B³ is a trivalent group comprising about 1 to about 20 atoms and is covalently bonded to T², T⁵, and T⁶;

T¹ is absent or a linking group;

T² is absent or a linking group;

T³ is absent or a linking group;

T⁴ is absent or a linking group;

T⁵ is absent or a linking group; and

T⁶ is absent or a linking group.

In certain embodiments, each saccharide is independently selected from:

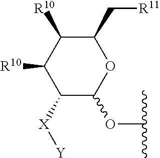

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

In certain embodiments, each the saccharide is independently selected from the group consisting of:

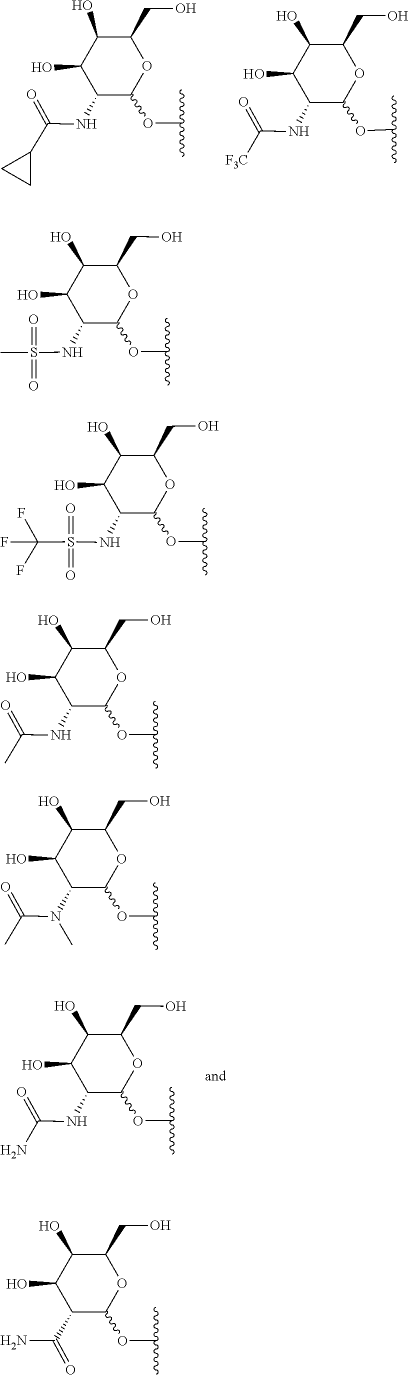

In certain embodiments, each saccharide is independently:

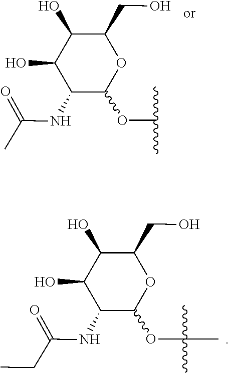

In certain embodiments, each of T³, T⁴, T⁵, and T⁶ is independently selected from the group consisting of:

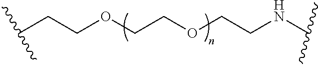

wherein:

n=1, 2, 3.

B¹ is CH;

B² is selected from the group consisting of:

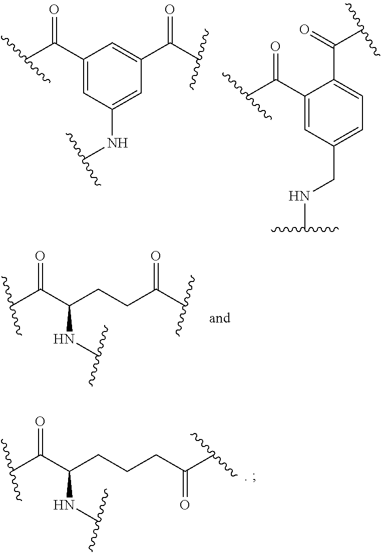 [SMILES: *Nc1cc(C(*)=O)cc(C(*)=O)c1 ; *NCc1ccc(C(*)=O)c(C(*)=O)c1 ; *N[C@H](CCC(*)=O)C(*)=O ; *N[C@H](CCCC(*)=O)C(*)=O]

and

B³ is selected from the group consisting of:

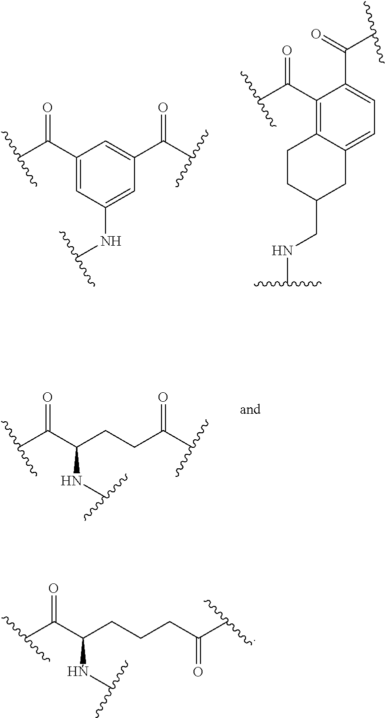 [SMILES: *Nc1cc(C(*)=O)cc(C(*)=O)c1 ; *NCC1CCc2c(ccc(C(*)=O)c2C(*)=O)C1 ; *N[C@H](CCC(*)=O)C(*)=O ; *C(=O)CCC[C@H](C(*)=O)N(*)C]

In certain embodiments, the nucleic acid is an oligonucleotide, and the conjugate is,

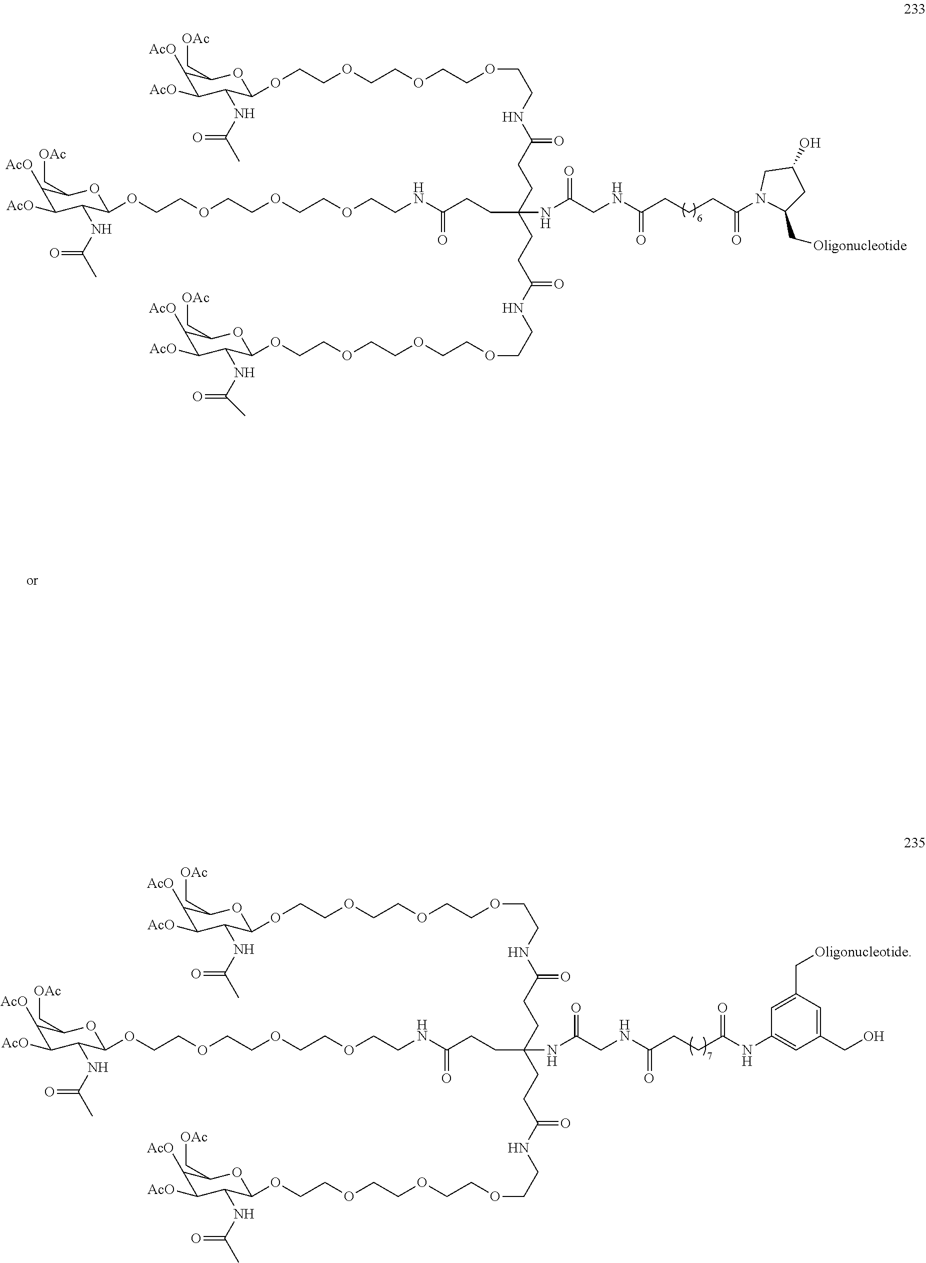

In certain embodiments, the conjugate is a conjugate of the following formula

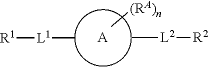

wherein the following definitions apply:

R¹ a is targeting ligand;

L¹ is absent or a linking group;

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen or a protecting group; and

n is 0, 1, 2, 3, 4, 5, 6, 7, 8, 9, or 10;

or a salt thereof.

In certain embodiments,

R¹ a is targeting ligand;

L¹ is absent or a linking group;

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B} and C₁₋₈ alkyl that is optionally substituted with one or more groups independently selected from halo, hydroxy, and C₁₋₃ alkoxy;

R^{B} is hydrogen or a protecting group; and

n is 0, 1, 2, 3, 4, 5, 6, 7, 8, 9, or 10.

In certain embodiments, R¹ is —C(H)₍₃₋ₚ₎(L³-saccharide)ₚ,

wherein each L³ is independently a linking group;

p is 1, 2, or 3; and

saccharide is a monosaccharide or disaccharide.

In certain embodiments, the saccharide is:

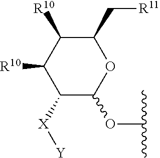

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

In certain embodiments, the saccharide is selected from the group consisting of:

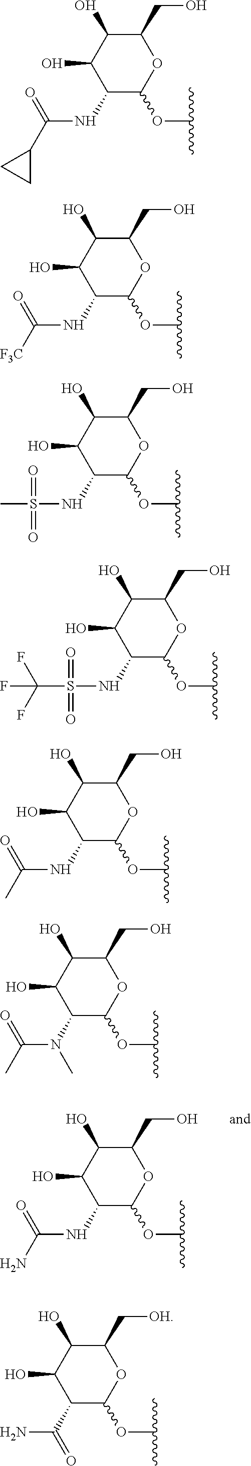

In certain embodiments, the saccharide is:

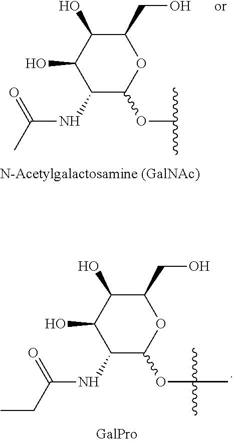 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In certain embodiments, each L³ is independently a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, each L³ is independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, L³ is:

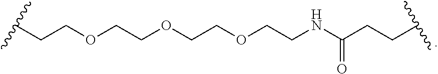 [SMILES: *CCOCCOCCOCCNC(=O)CC*]

In certain embodiments, R¹ is:

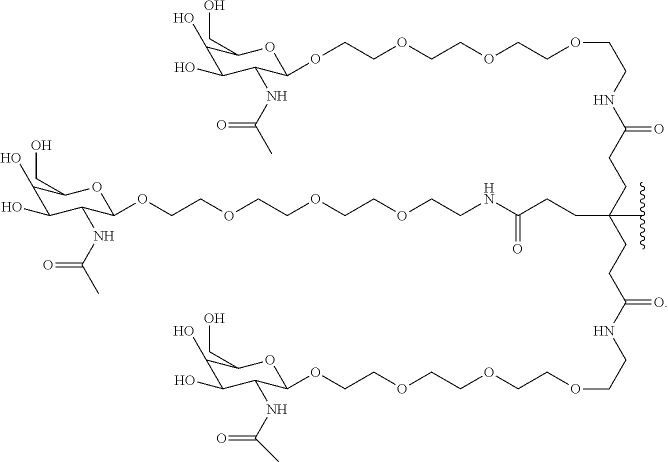 [SMILES: *C(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In certain embodiments, R¹ is:

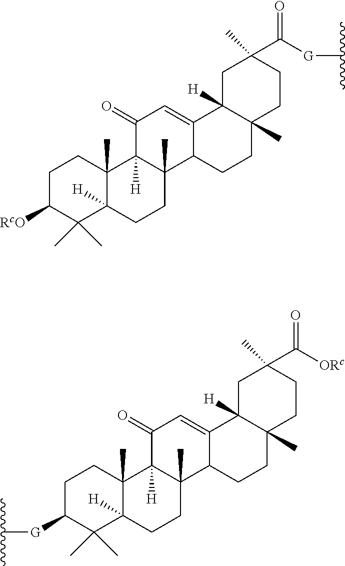

wherein:

G is —NH— or —O—;

R^{C} is hydrogen, (C₁-C₈)alkyl, (C₁-C₈)haloalkyl, (C₁-C₈)alkoxy, (C₁-C₆)alkanoyl, (C₃-C₂₀)cycloalkyl, (C₃-C₂₀)heterocycle, aryl, heteroaryl, monosaccharide, disaccharide or trisaccharide; and wherein the cycloalkyl, heterocyle, ary, heteroaryl and saccharide are optionally substituted with one or more groups independently selected from the group consisting of halo, carboxyl, hydroxyl, amino, (C₁-C₄)alkyl, (C₁-C₄)haloalkyl, (C₁-C₄)alkoxy and (C₁-C₄)haloalkoxy.

In certain embodiments, R^{C} is:

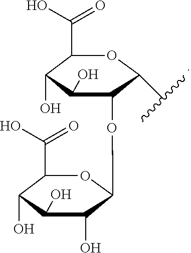

In certain embodiments, R¹ is:

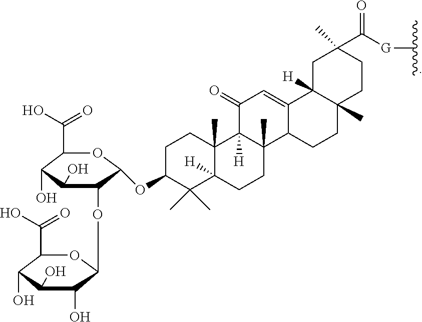

In certain embodiments, R^{C} is:

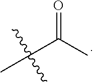 [SMILES: *C(C)=O]

In certain embodiments, G is —NH—.

In certain embodiments, R¹ is:

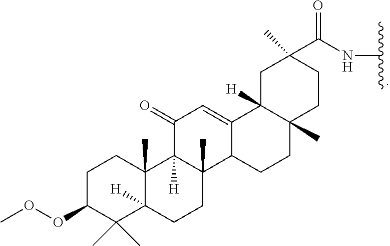

In certain embodiments, R¹ is:

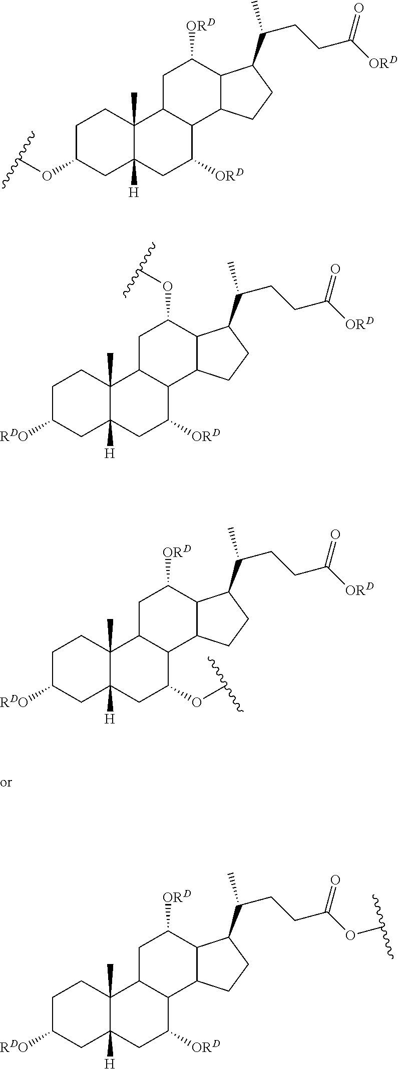

wherein each R^{D} is independently selected from the group consisting of hydrogen, (C₁-C₆)alkyl, (C₉-C₂₀)alkylsilyl, (R^{W})₃Si—, (C₂-C₆)alkenyl, tetrahydropyranyl, (C₁-C₆)alkanoyl, benzoyl, aryl(C₁-C₃)alkyl, TMTr (Trimethoxytrityl), DMTr (Dimethoxytrityl), MMTr (Monomethoxytrityl), and Tr (Trityl); and

each R^{W} is independently selected from the group consisting of (C₁-C₄)alkyl and aryl.

In certain embodiments, L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, L¹ and L² are independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, L¹ and L² are independently, a divalent, branched or unbranched, saturated or unsaturated, hydrocarbon chain, having from 1 to 14 carbon atoms, wherein one or more (e.g. 1, 2, 3, or 4) of the carbon atoms in the hydrocarbon chain is optionally replaced —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, L¹ is connected to R¹ through —NH—, —O—, —S—, —(C═O)—, —(C═O)—NH—, —NH—(C═O)—, —(C═O)—O—, —NH—(C═O)—NH—, or —NH—(SO₂)—.

In certain embodiments, L² is connected to R² through —O—.

In certain embodiments, L¹ is selected from the group consisting of:

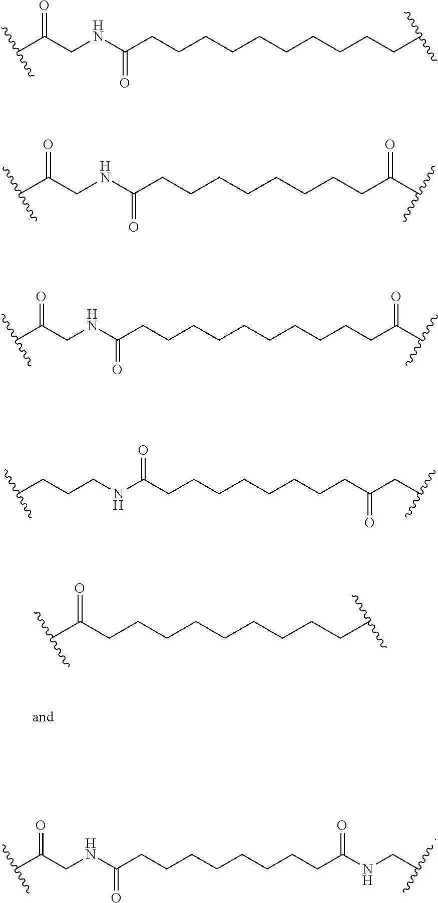 [SMILES: *CCCCCCCCCCC(=O)NCC(*)=O ; *C(=O)CCCCCCCCC(=O)NCC(*)=O ; *C(=O)CCCCCCCCCCC(=O)NCC(*)=O ; *CCCNC(=O)CCCCCCCCC(=O)C* ; *CCCCCCCCCC(*)=O ; *CNC(=O)CCCCCCCCC(=O)NCC(*)=O]

In certain embodiments, L² is —CH₂—O— or —CH₂—CH₂—O—.

In certain embodiments, the conjugate is a conjugate of the following formula:

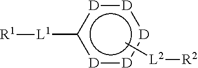

wherein: each D is independently selected from the group consisting of

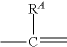

### and —N═.

In certain embodiments, the conjugate is selected from the group consisting of:

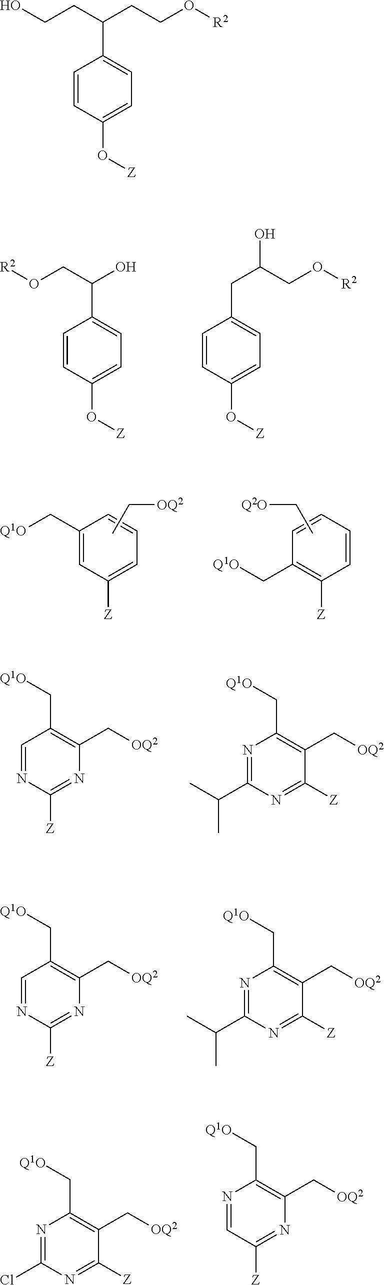

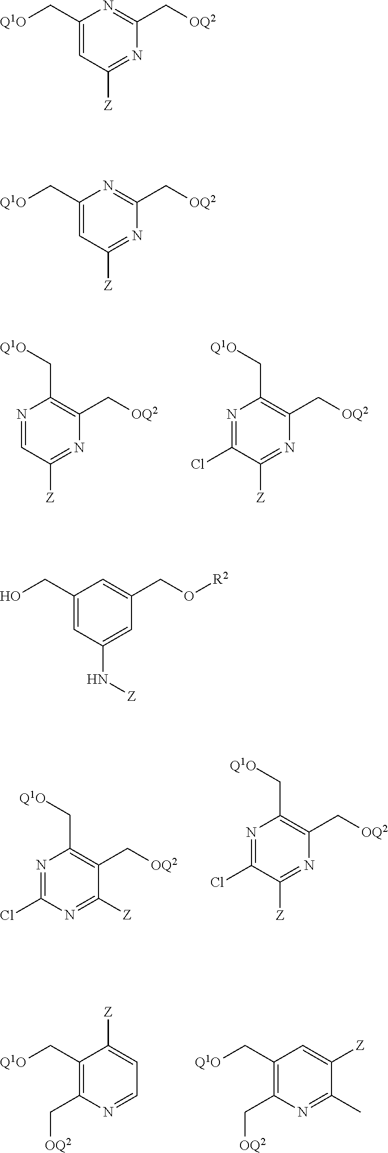

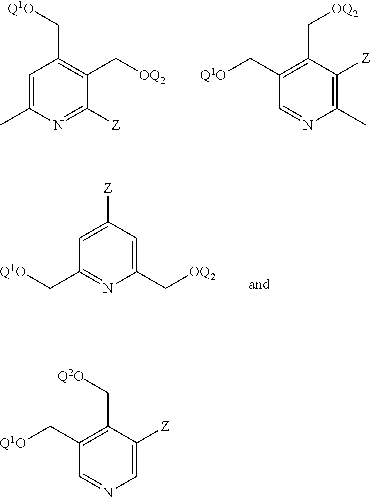

wherein:

Q¹ is hydrogen and Q² is R²; or Q¹ is R² and Q² is hydrogen; and

Z is -L¹-R¹.

In certain embodiments, the conjugate is a conjugate of the following formula:

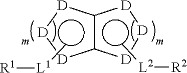

wherein: each D is independently selected from the group consisting of

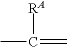

### and —N═; and

each m is independently 1 or 2.

In certain embodiments, the conjugate is selected from the group consisting of:

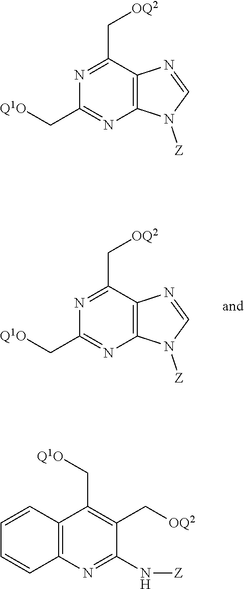

wherein:

Q¹ is hydrogen and Q² is R²; or Q¹ is R² and Q² is hydrogen; and

Z is -L¹-R¹.

In certain embodiments, the conjugate is a conjugate of the following formula:

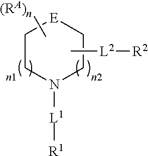

wherein:

E is —O— or —CH₂—;

n is selected from the group consisting of 0, 1, 2, 3, and 4; and

n1 and n2 are each independently selected from the group consisting of 0, 1, 2, and 3.

In certain embodiments, the conjugate is a conjugate is selected from the group consisting of:

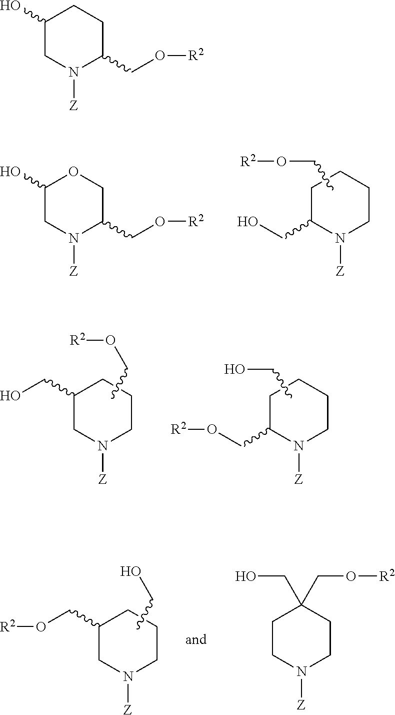

wherein: Z is -L¹-R¹.

In certain embodiments, the -A-L²-R² moiety is:

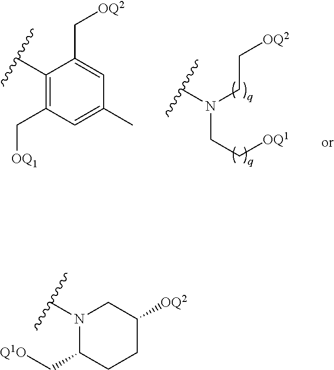

wherein:

Q¹ is hydrogen and Q² is R²; or Q¹ is R² and Q² is hydrogen; and

each q is independently 0, 1, 2, 3, 4 or 5.

In certain embodiments, R² is an oligonucleotide.

In certain embodiments, R² is an siRNA.

In certain embodiments, the conjugate is selected from the group consisting of:

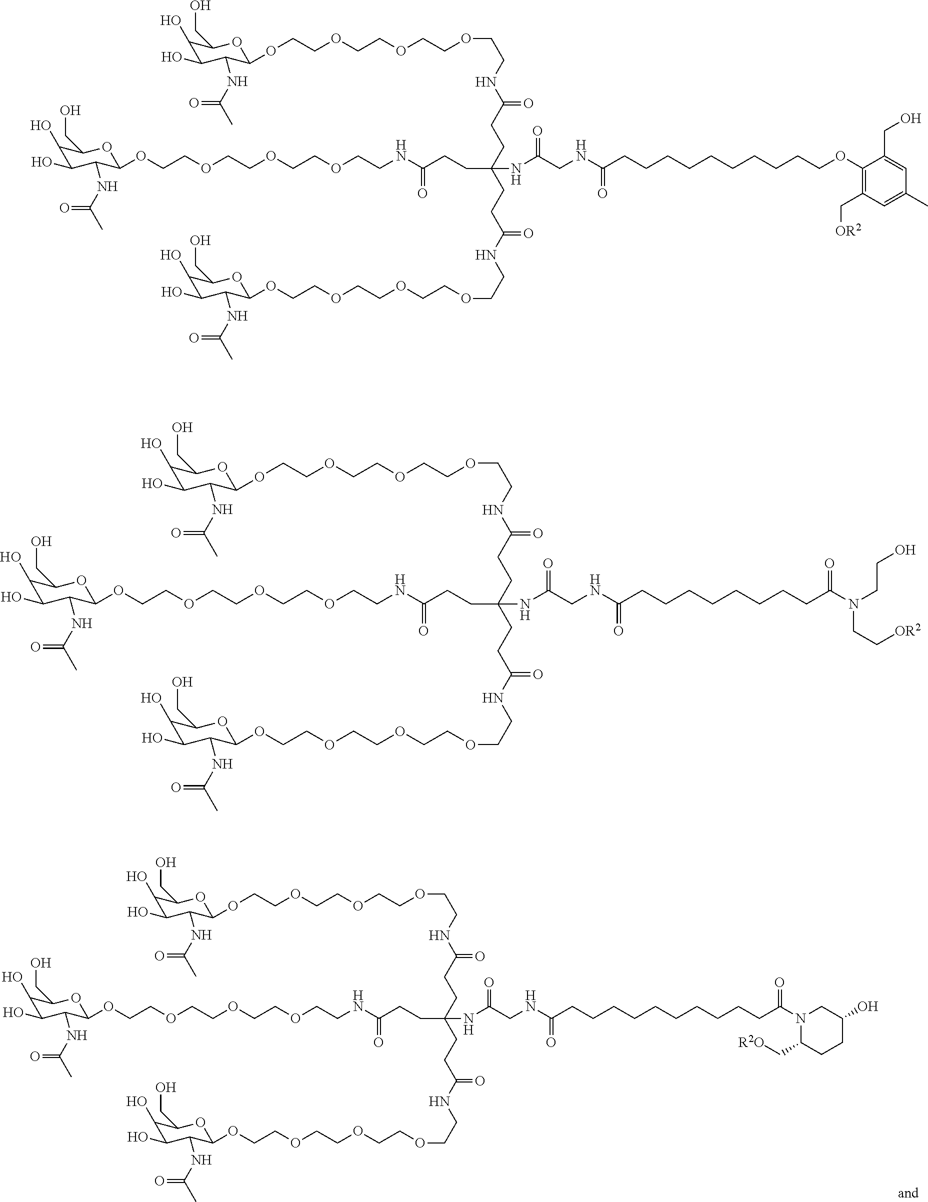

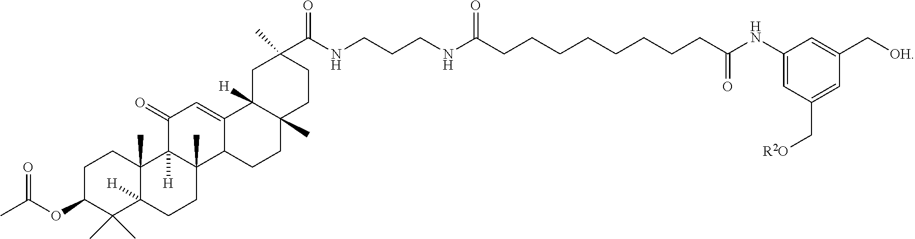

In certain embodiments, R¹ is selected from the group consisting of:

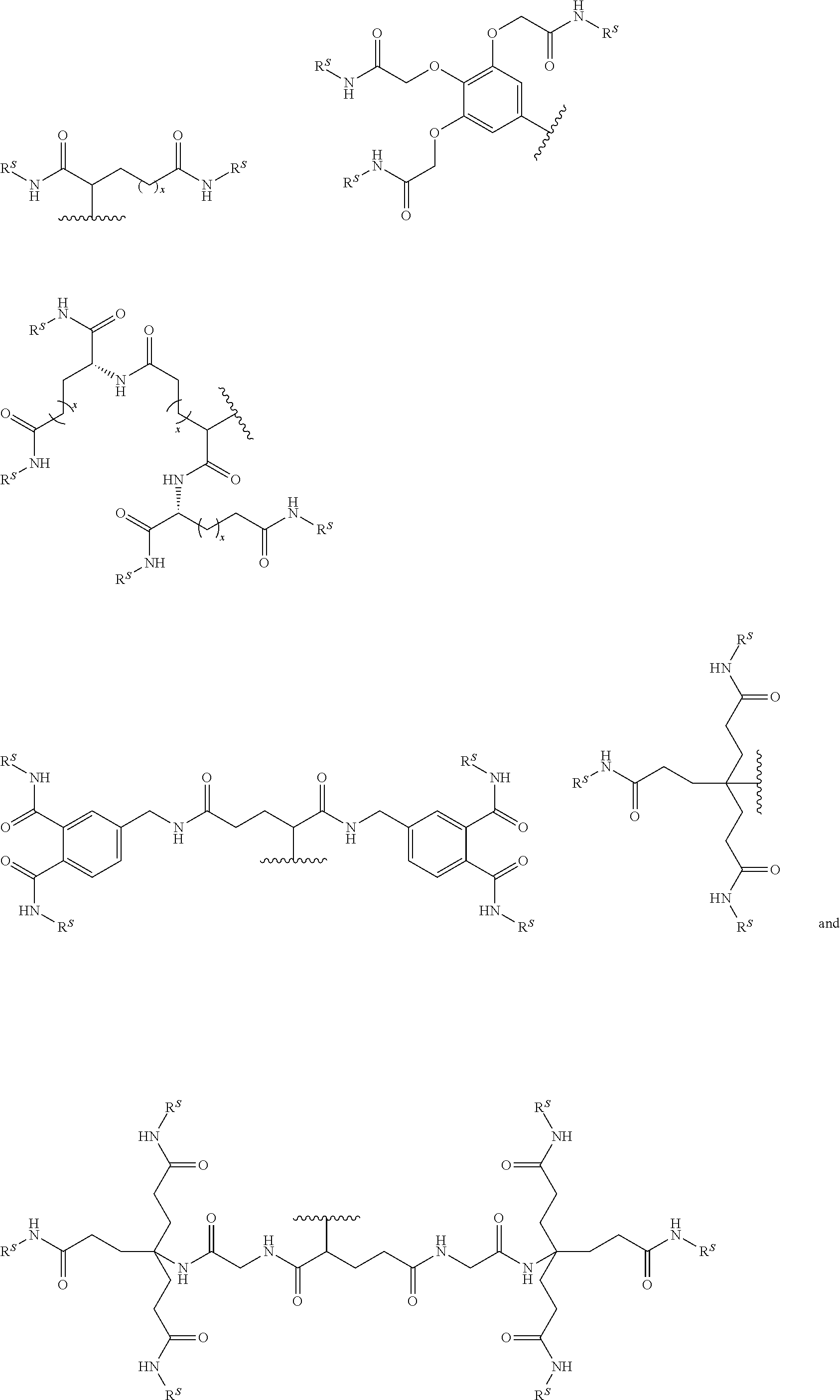

wherein:

R^{S} is

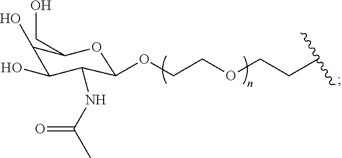

n is 2, 3, or 4; and

x is 1 or 2.

In certain embodiments, L¹ is selected from the group consisting of:

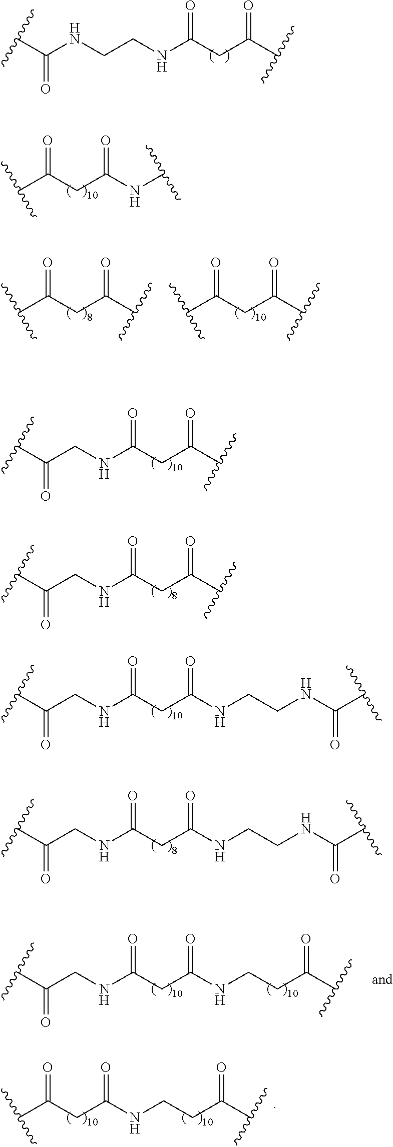

In certain embodiments, A is absent, phenyl, pyrrolidinyl, or cyclopentyl.

In certain embodiments, L² is C₁₋₄ alkylene-O— that is optionally substituted with hydroxy.

In certain embodiments, L² is —CH₂O—, —CH₂CH₂O—, or —CH(OH)CH₂O—.

In certain embodiments, each R^{A} is independently hydroxy or C₁₋₈ alkyl that is optionally substituted with hydroxyl.

In certain embodiments, each R^{A} is independently selected from the group consisting of hydroxy, methyl and —CH₂OH.

In certain embodiments, the conjugate is a conjugate of the following formula:

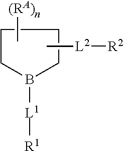

wherein:

B is —N— or —CH—;

L² is C₁₋₄ alkylene-O— that is optionally substituted with hydroxyl or halo; and

n is 0, 1, 2, 3, 4, 5, 6, or 7.

In certain embodiments, the conjugate is selected from the group consisting of:

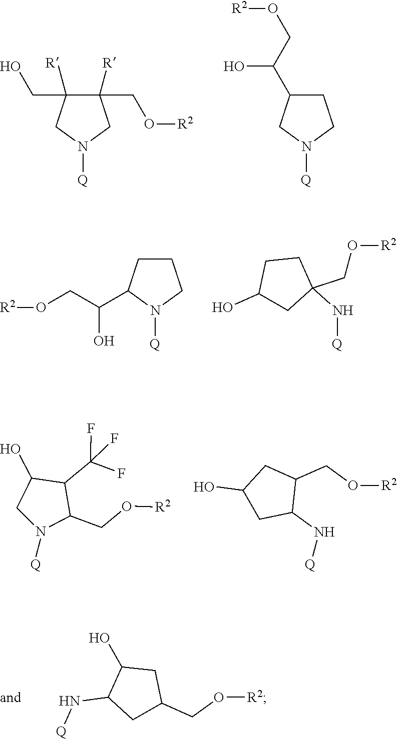

wherein Q is -L¹-R¹; and

R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

In certain embodiments, the conjugate is selected from the group consisting of:

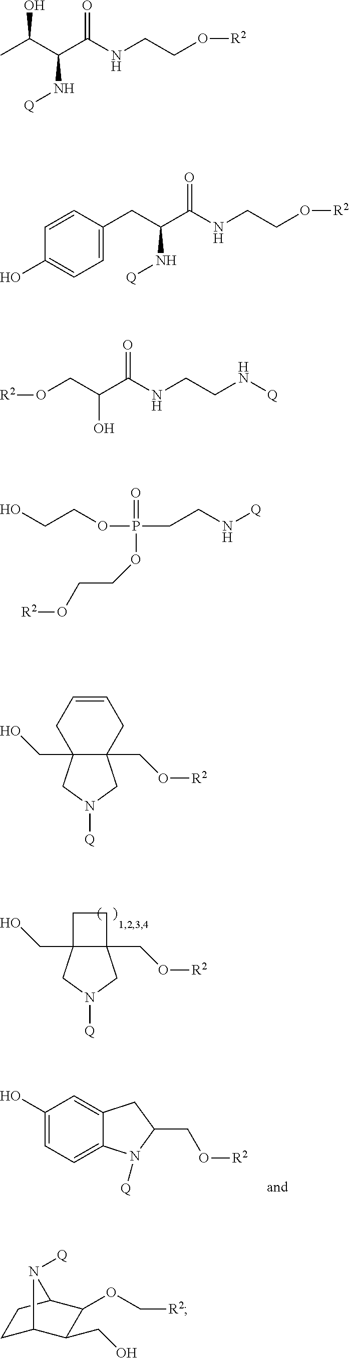

wherein Q is -L¹-R¹.

In certain embodiments, the conjugate is selected from the group consisting of:

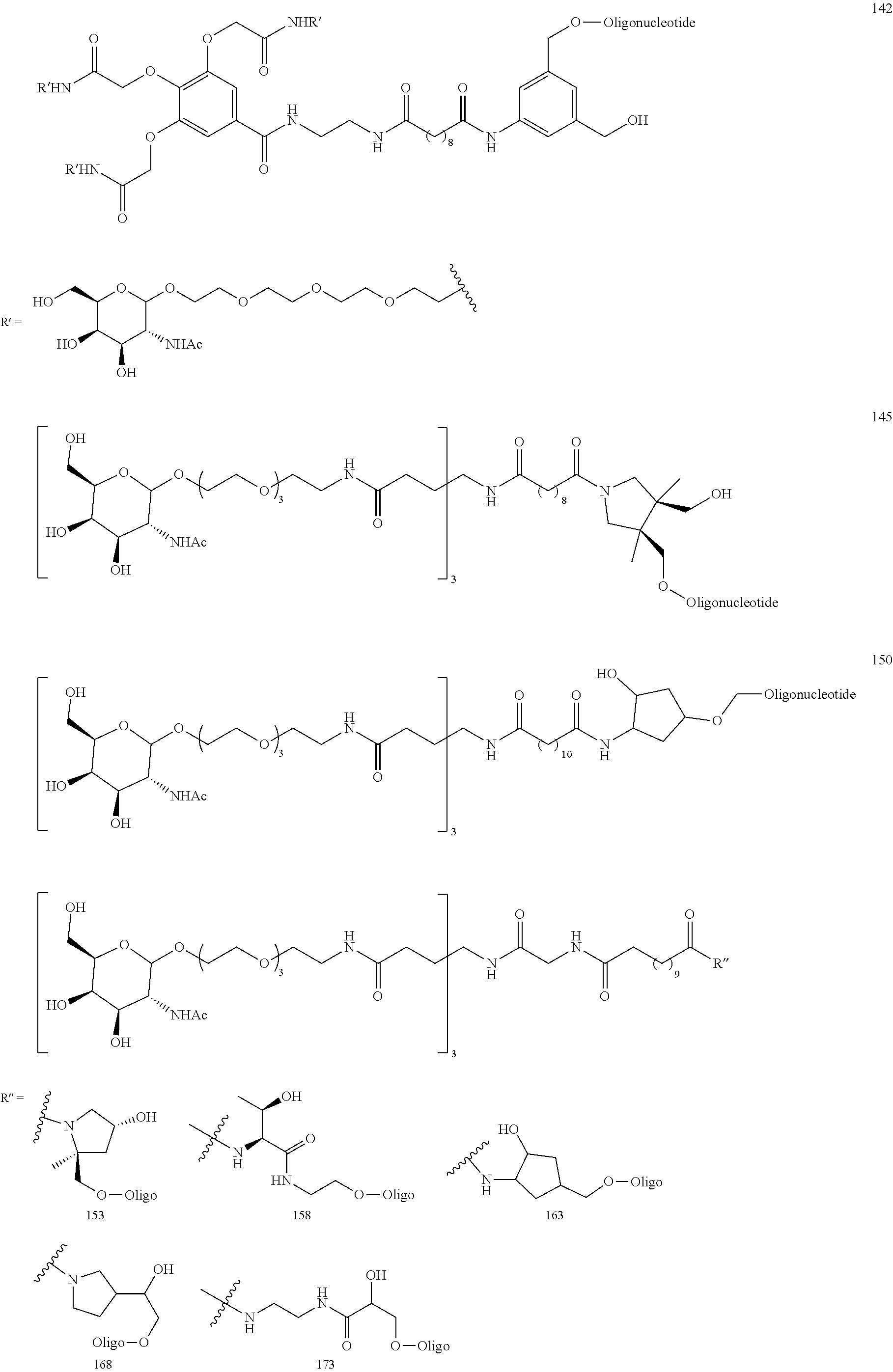

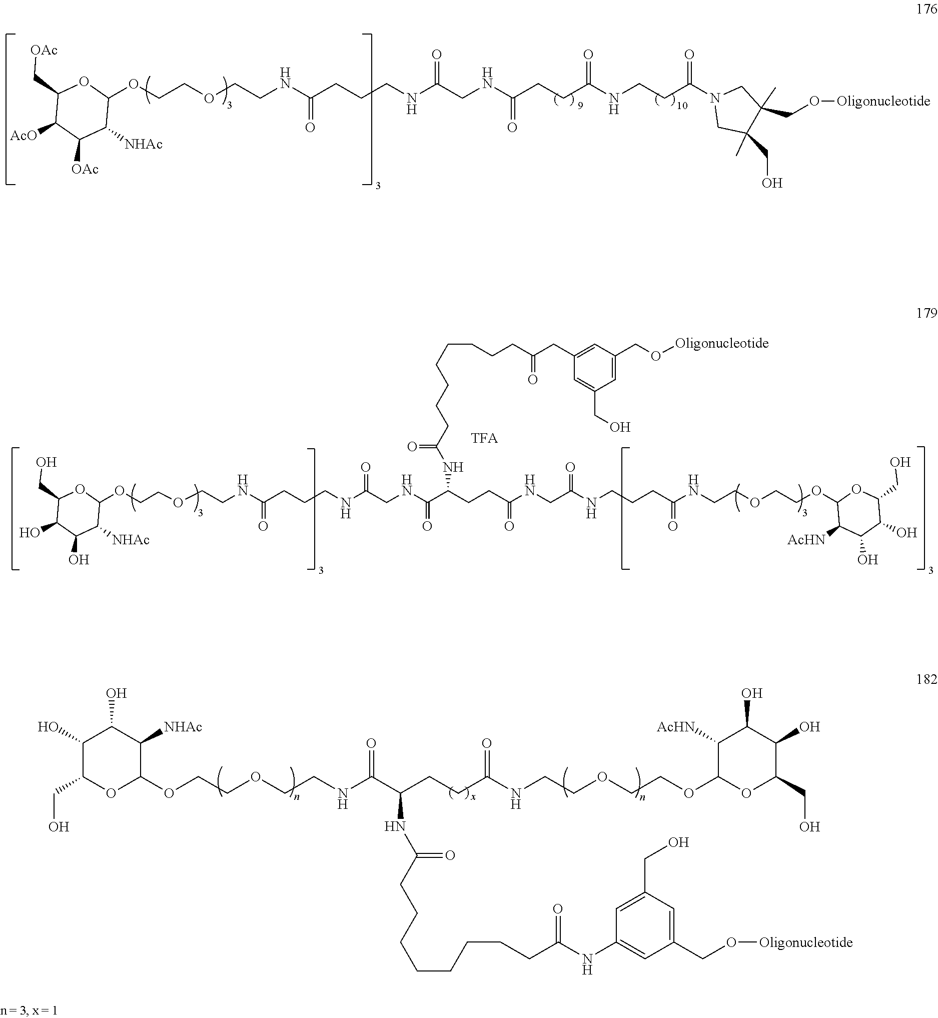

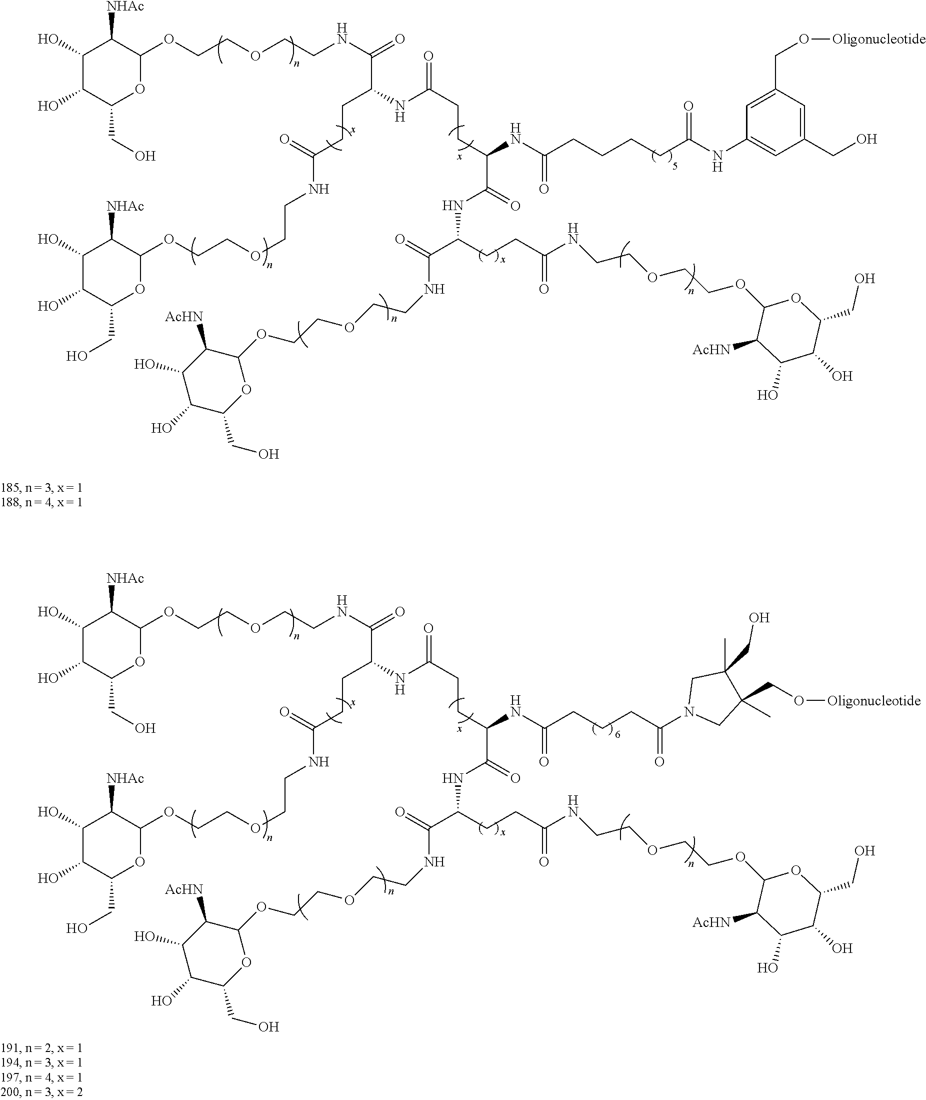

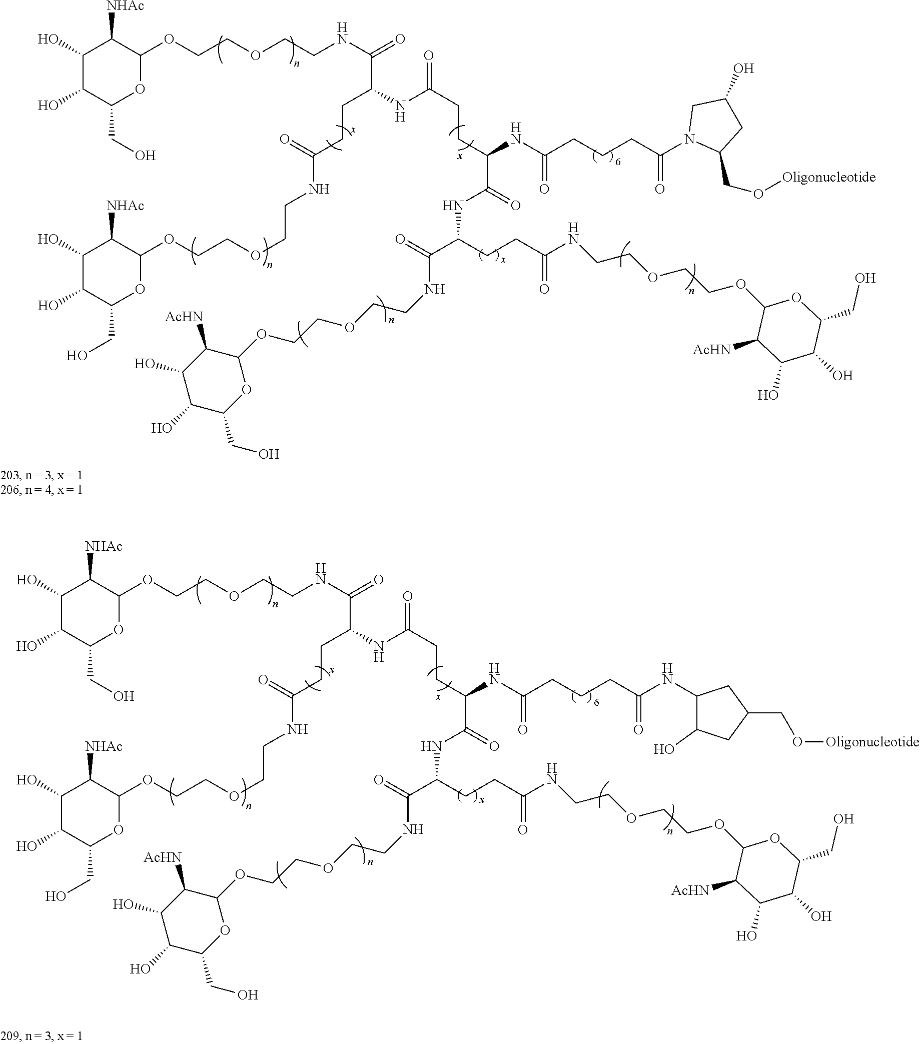

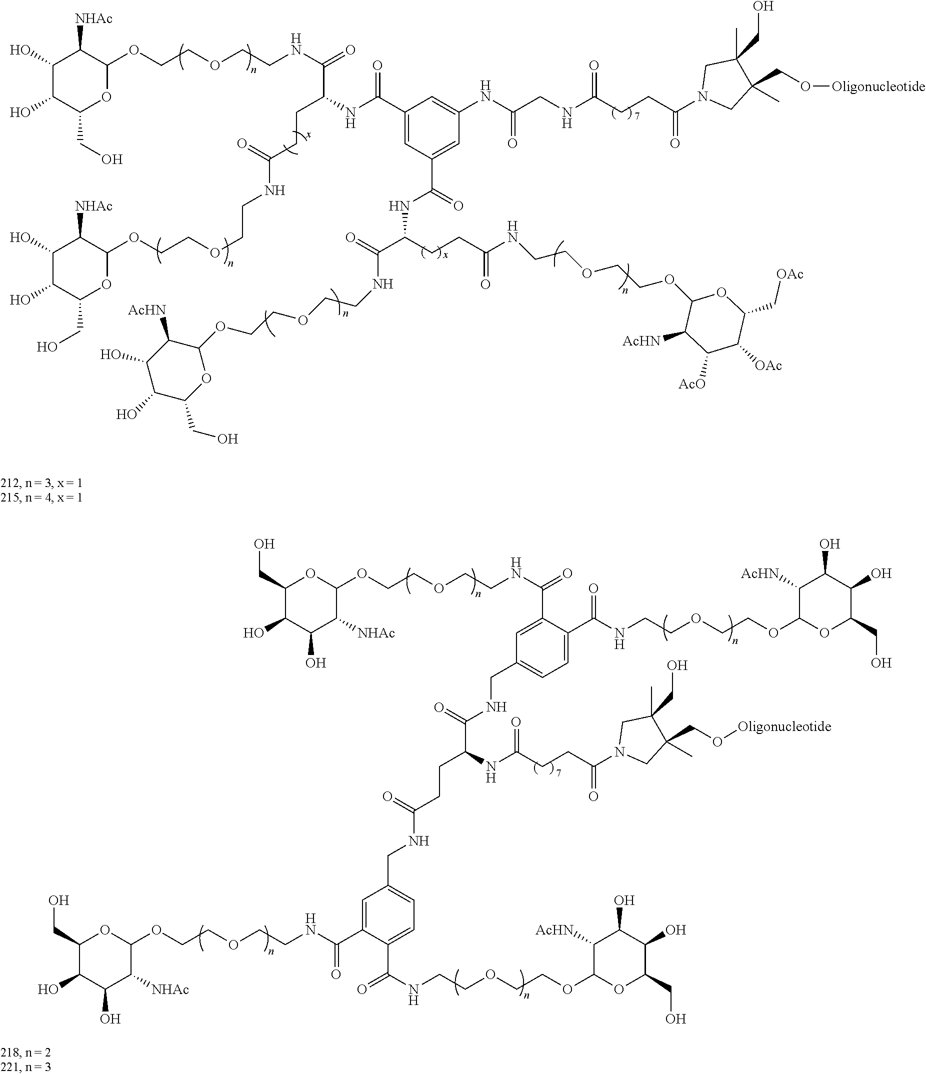

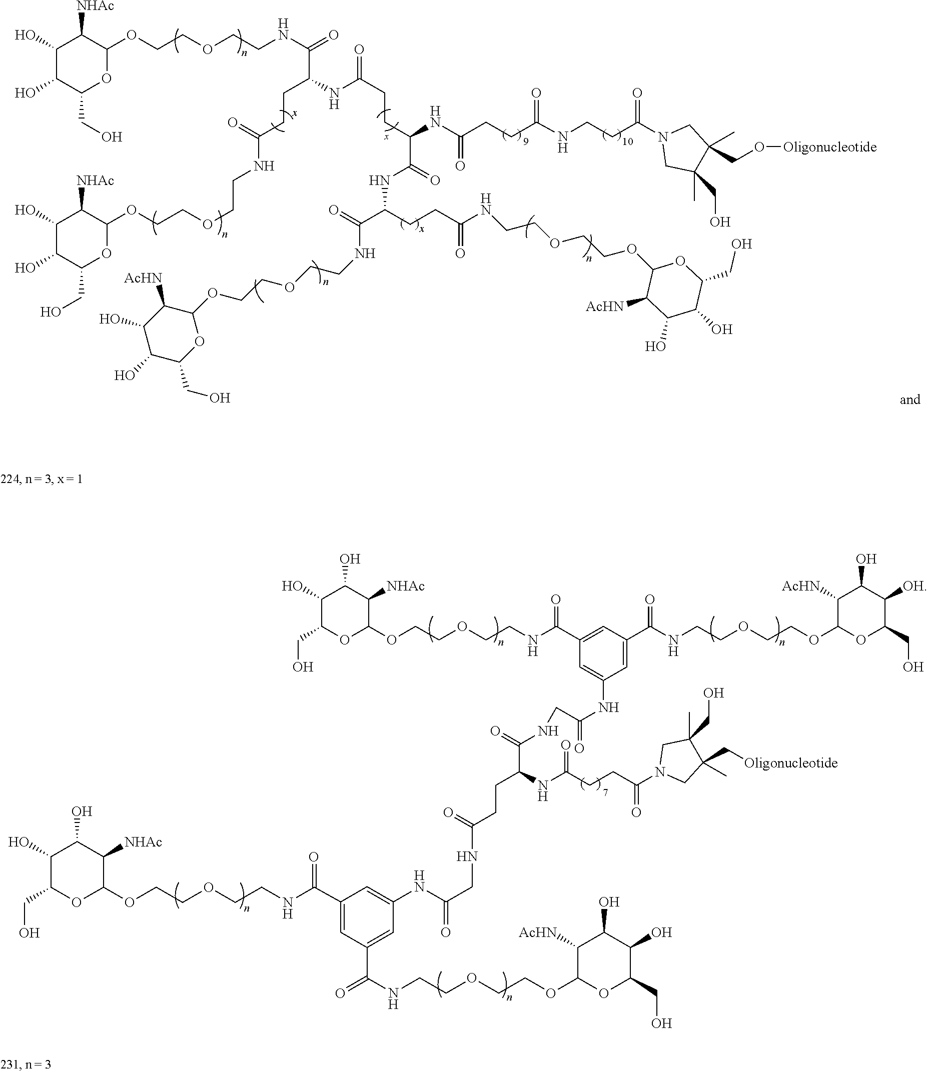

In certain embodiments, the conjugate is a conjugate of the following formula:

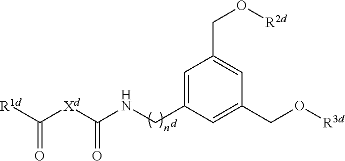

wherein the following definitions apply:

R^{1d} is selected from:

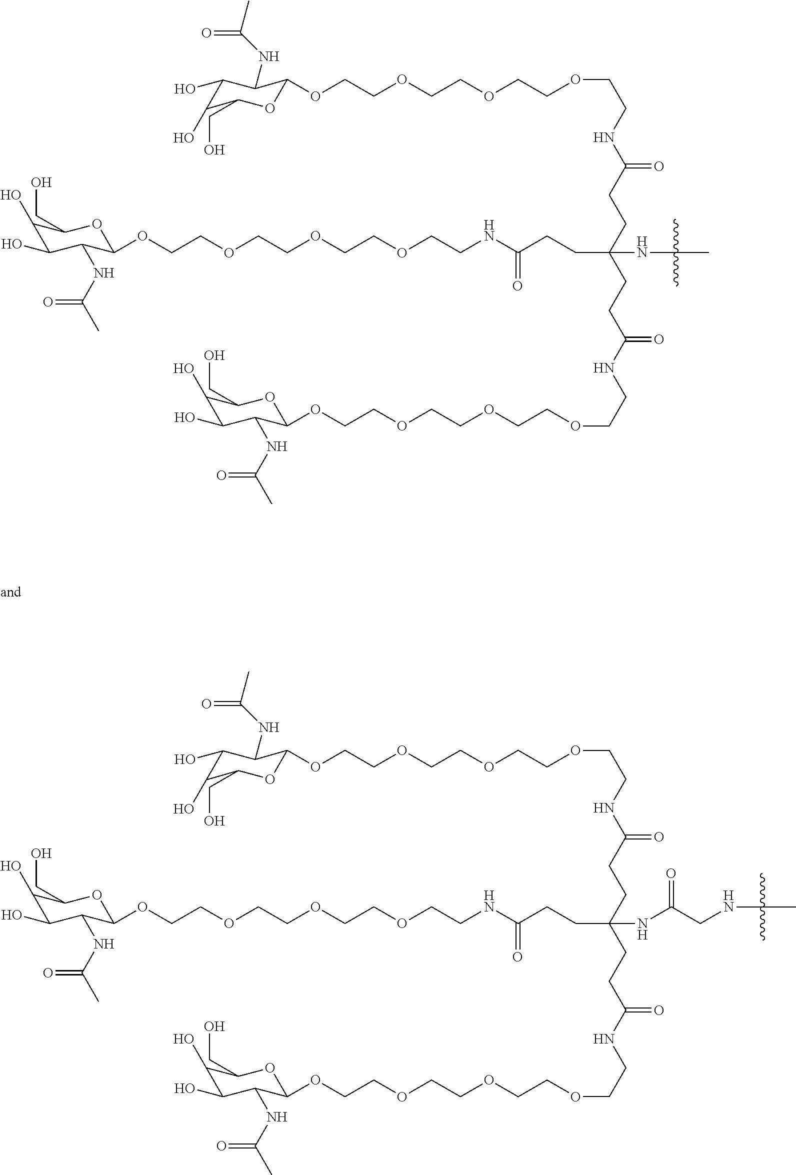 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

X^{d} is C₂₋₁₀ alkylene;

n^{d} is 0 or 1;

R^{2d} is a nucleic acid; and

R^{3d} is H or a protecting group.

In certain embodiments, R^{1d} is:

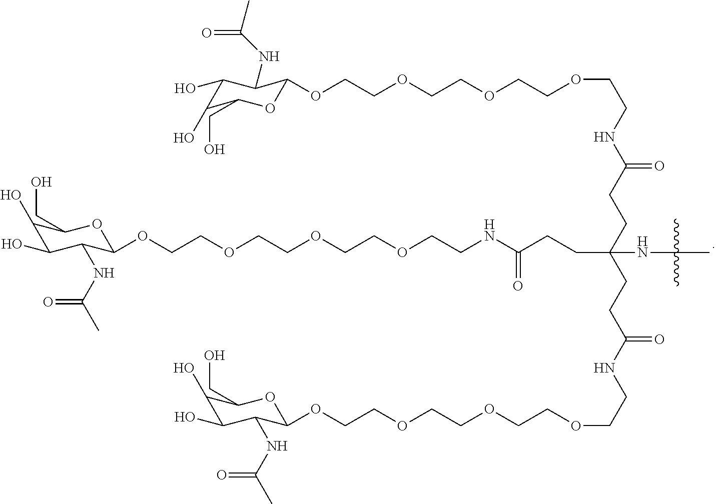 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In certain embodiments, R^{1d} is:

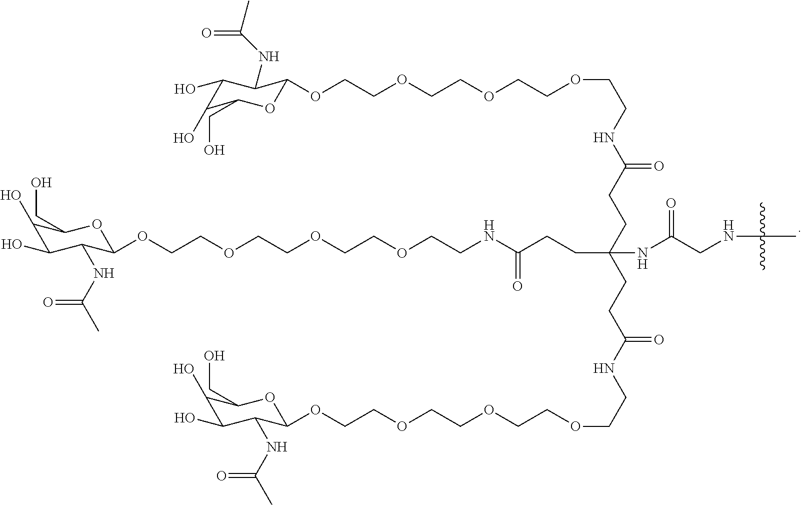 [SMILES: *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In certain embodiments, X^{d} is C₈alkylene.

In certain embodiments, n^{d} is 0.

In certain embodiments, R^{2d} is an siRNA.

In certain embodiments, R^{3d} is H.

In certain embodiments, the conjugate is a conjugate of the following formula:

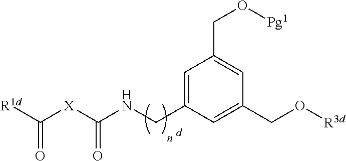

wherein the following definitions apply:

R^{1d} is selected from:

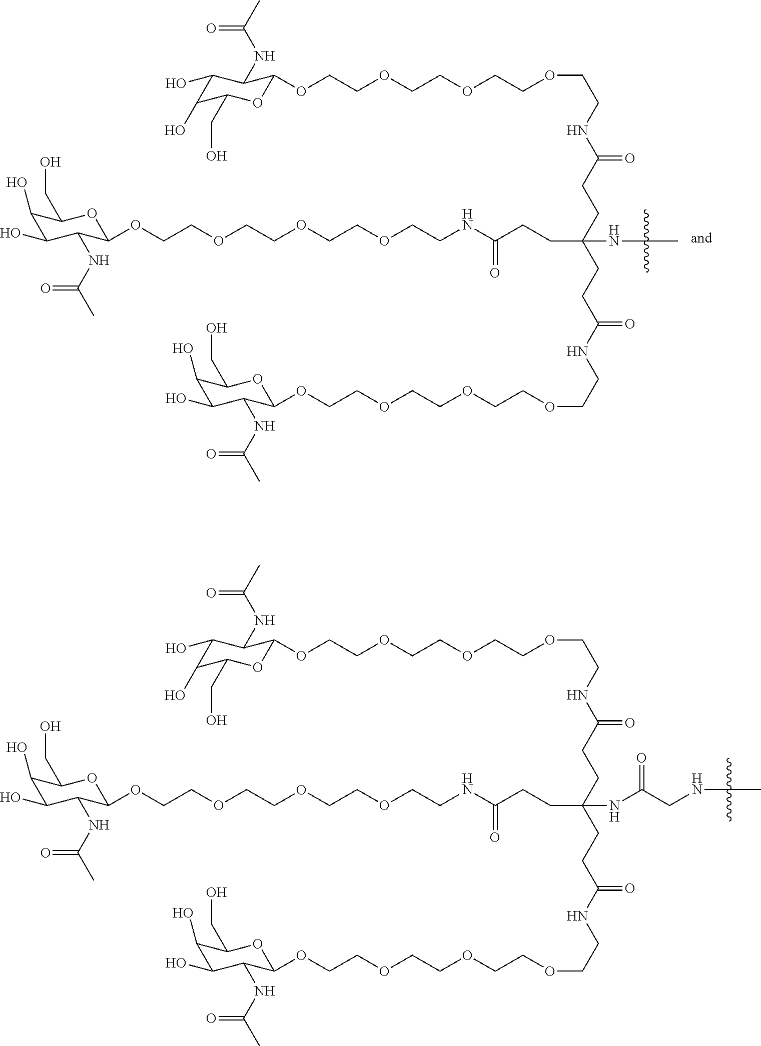 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

X^{d} is C₂₋₈ alkylene;

n^{d} is 0 or 1;

Pg¹ is H or a suitable protecting group; and

R^{3d} is H or a protecting group.

In certain embodiments, Pg¹ is TMTr (Trimethoxytrityl), DMTr (Dimethoxytrityl), MMTr (Monomethoxytrityl), or Tr (Trityl).

In certain embodiments, the conjugate is selected from the group consisting of:

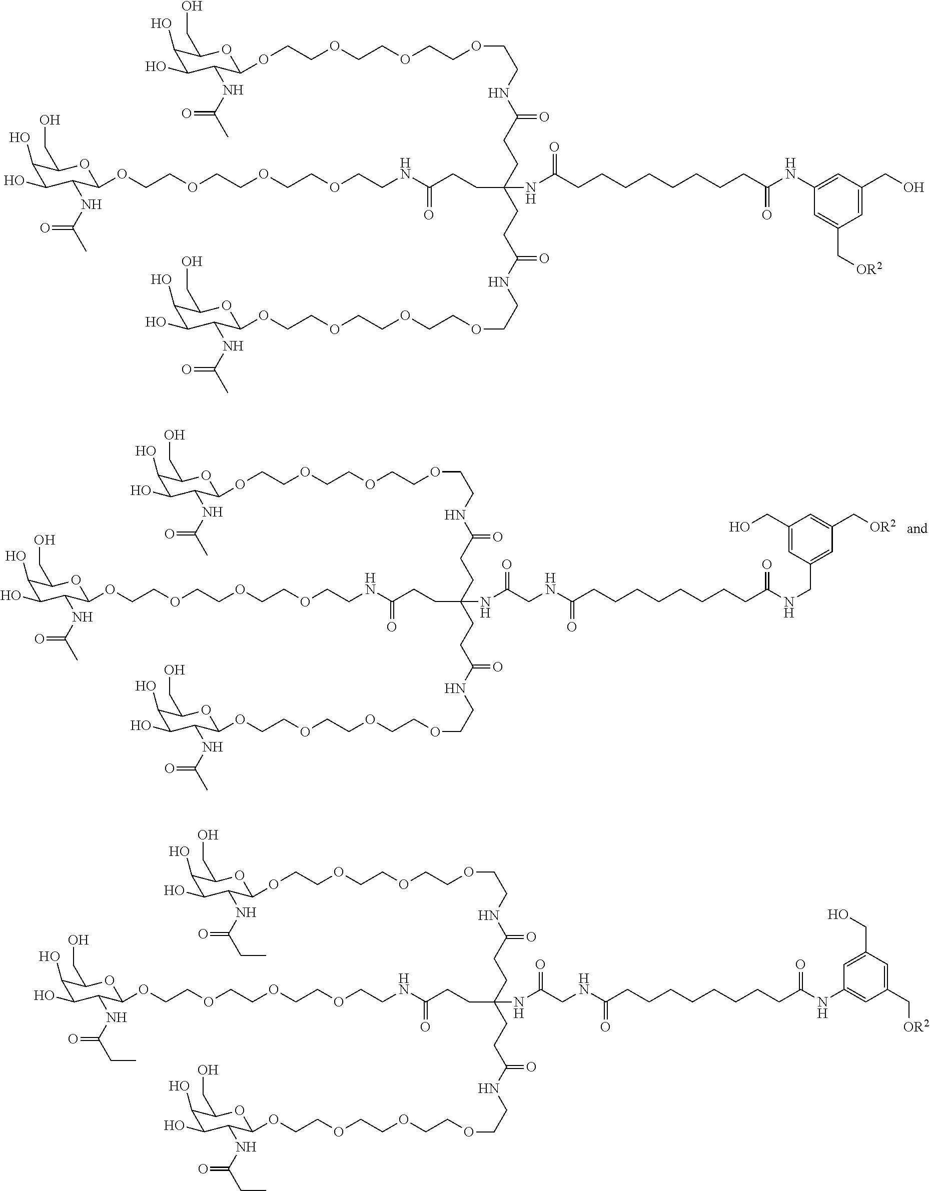

In certain embodiments, the conjugate is a conjugate of the following formula:

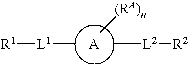

wherein the following definitions apply:

R¹ is H or a synthetic activating group;

L¹ is absent or a linking group;

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen or a protecting group; and

n is 0, 1, 2, 3, 4, 5, 6, 7, 8, 9, or 10.

In certain embodiments, the conjugate is a conjugate of the following formula

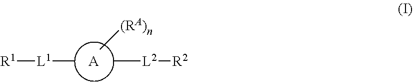

wherein the following definitions apply:

R¹ a is targeting ligand;

L¹ is absent or a linking group;

L² is absent or a linking group;

R² is H or a synthetic activating group;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen, or a protecting group; and

n is 0, 1, 2, 3, 4, 5, 6, 7, 8, 9, or 10.

In certain embodiments, the conjugate is a conjugate of the following formula

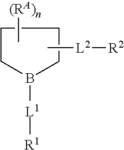

wherein:

B is —N— or —CH—;

L² is C₁₋₄ alkylene-O— that is optionally substituted with hydroxyl or halo; and

n is 0, 1, 2, 3, 4, 5, 6, or 7.

In certain embodiments the conjugate is selected from the group consisting of:

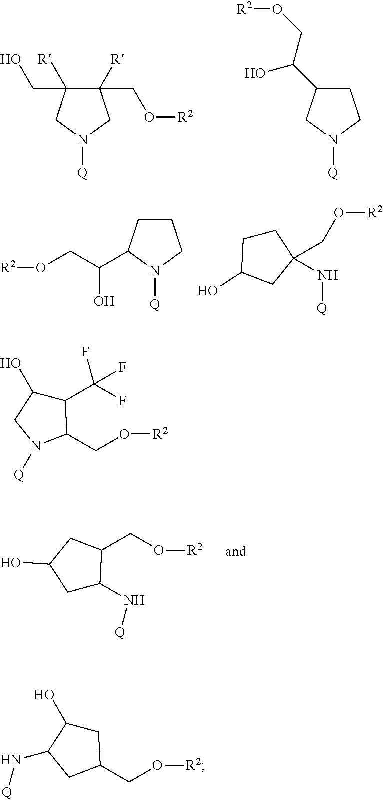

wherein:

Q is -L¹-R¹; and

R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

In certain embodiments, the conjugate is selected from the group consisting of:

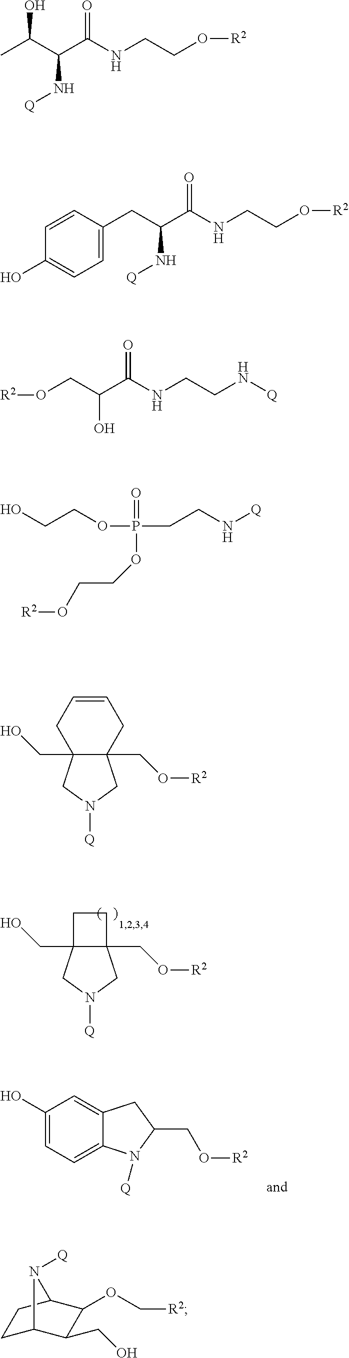

wherein: Q is -L¹-R¹.

In certain embodiments, the conjugate is a conjugate of the following formula:

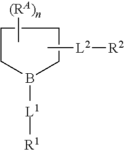

wherein the following definitions apply:

B is —N— or —CH—;

L¹ is absent or a linking group;

L² is C₁₋₄ alkylene-O— that is optionally substituted with hydroxyl or halo;

n is 0, 1, 2, 3, 4, 5, 6, or 7;

R¹ is H or a synthetic activating group; and

R² is H or a synthetic activating group.

In certain embodiments, the conjugate is selected from the group consisting of:

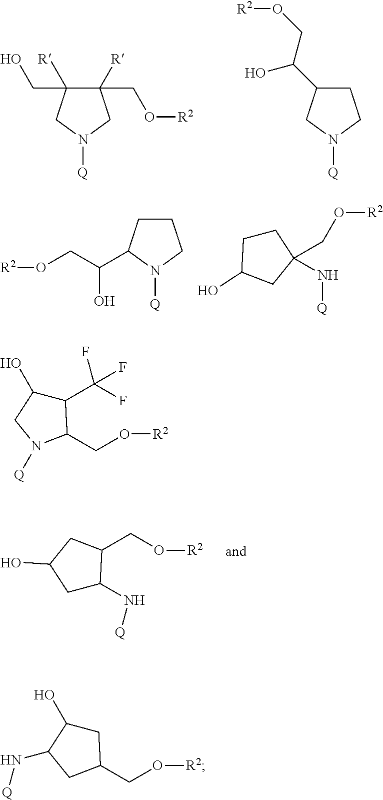

wherein Q is -L¹-R¹;

L¹ is absent or a linking group;

R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;

R¹ is H or a synthetic activating group; and

R² is H or a synthetic activating group.

In certain embodiments, the conjugate is selected from the group consisting of:

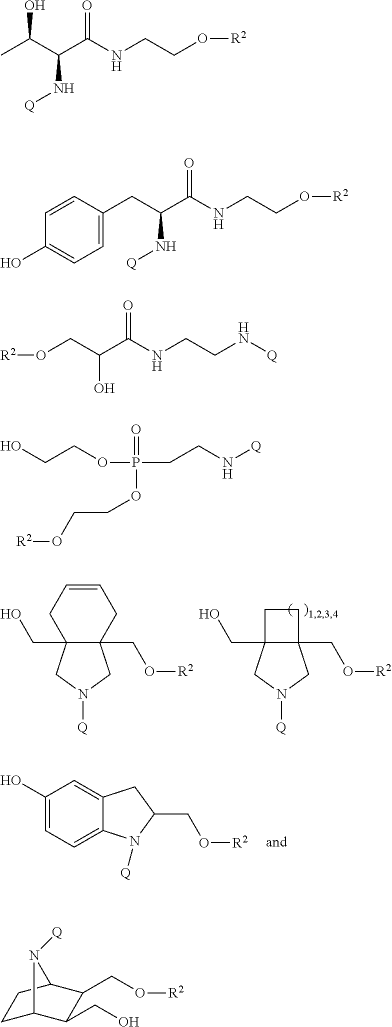

wherein:

Q is -L¹-R¹;

L¹ is absent or a linking group;

R¹ is H or a synthetic activating group; and

R² is H or a synthetic activating group.

In certain embodiments, R¹ is H or a synthetic activating group derivable from DCC, HOBt, EDC, BOP, PyBOP or HBTU.

In certain embodiments, R² is H, acetate, triflate, mesylate or succinate.

In certain embodiments, R¹ is a synthetic activating group derivable from DCC, HOBt, EDC, BOP, PyBOP or HBTU.

In certain embodiments, R² is acetate, triflate, mesylate or succinate.

In certain embodiments, L¹ is a divalent, branched or unbranched, saturated or unsaturated, hydrocarbon chain, having from 5 to 20 carbon atoms, wherein one or more (e.g. 1, 2, 3, or 4) of the carbon atoms in the hydrocarbon chain is optionally replaced —O—, —NH—, —NH—C(═O)—, —C(═O)—NH— or —S—.

In certain embodiments, the conjugate is a conjugate of the following formula:

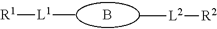

wherein the following definitions apply:

R¹ a is targeting ligand;

L¹ is absent or a linking group;

L² is absent or a linking group;

R² is a nucleic acid;

B is divalent and is selected from the group consisting of:

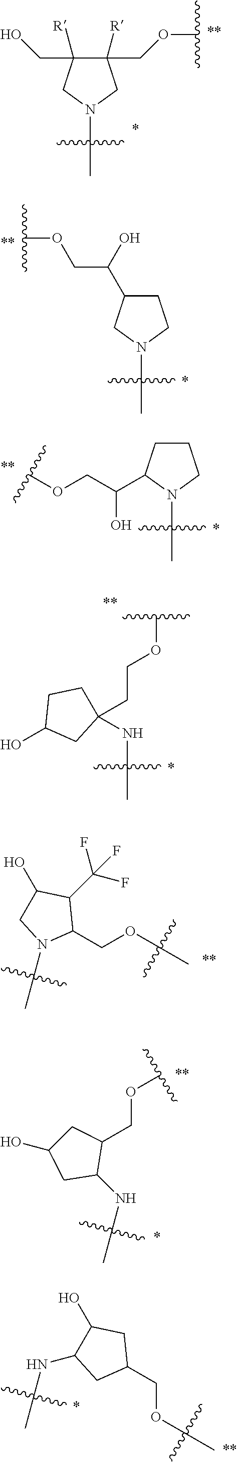

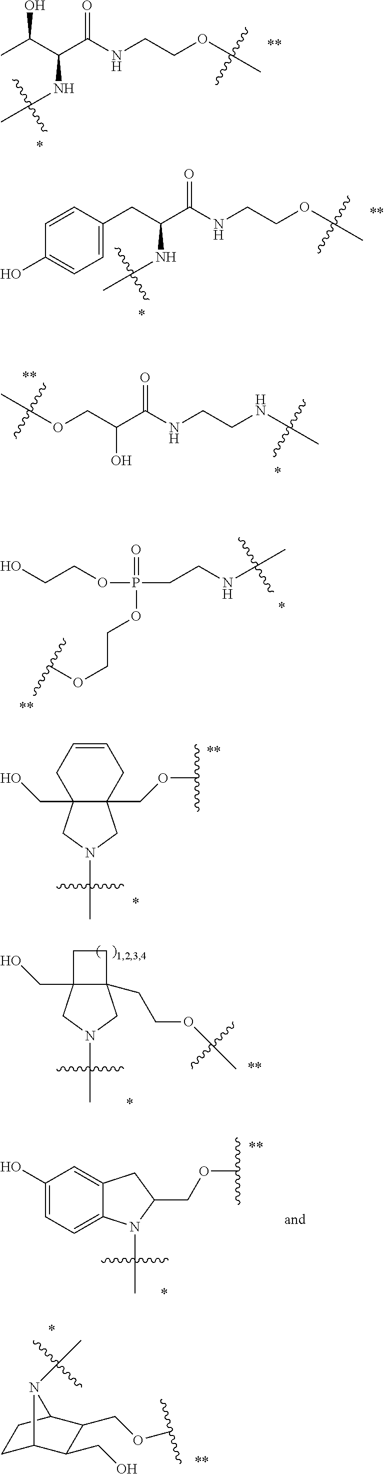

wherein:

each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;

the valence marked with * is attached to L¹ or is attached to R¹ if L¹ is absent; and

the valence marked with ** is attached to L² or is attached to R² if L² is absent;

or a salt thereof.

In certain embodiments, the targeting ligand R¹ comprises 2-8 saccharides.

In certain embodiments, the targeting ligand R¹ comprises 2-4 saccharides.

In certain embodiments, the targeting ligand R¹ comprises 3-8 saccharides.

In certain embodiments, the targeting ligand R¹ comprises 3-6 saccharides.

In certain embodiments, the targeting ligand R¹ comprises 3-4 saccharides.

In certain embodiments, the targeting ligand R¹ comprises 3 saccharides.

In certain embodiments, the targeting ligand R¹ comprises 4 saccharides.

In certain embodiments, and as it may be applied to any of the conjugate definitions, the targeting moiety R¹ has the following formula:

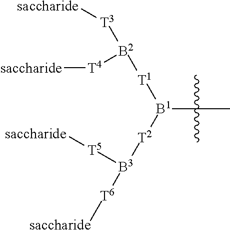

wherein:

B¹ is a trivalent group comprising about 1 to about 20 atoms and is covalently bonded to L¹, T¹, and T².

B² is a trivalent group comprising about 1 to about 20 atoms and is covalently bonded to T¹, T³, and T⁴;

B³ is a trivalent group comprising about 1 to about 20 atoms and is covalently bonded to T², T⁵, and T⁶;

T¹ is absent or a linking group;

T² is absent or a linking group;

T³ is absent or a linking group;

T⁴ is absent or a linking group;

T⁵ is absent or a linking group; and

T⁶ is absent or a linking group.

In certain embodiments, each saccharide is independently selected from:

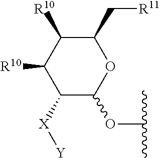

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

In certain embodiments, each the saccharide is independently selected from the group consisting of:

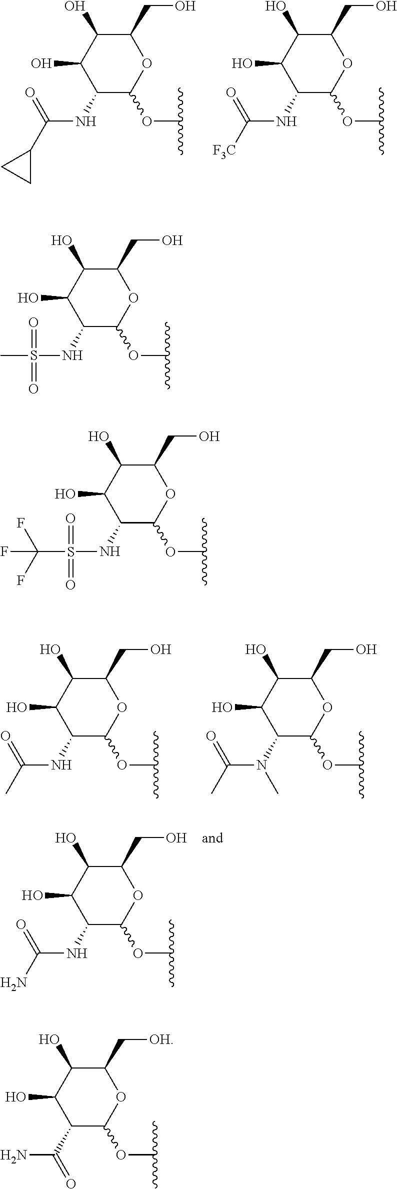

In certain embodiments, each saccharide is independently:

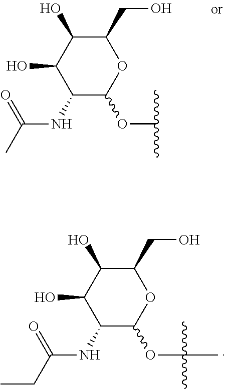 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In certain embodiments, one of T¹ and T² is absent.

In certain embodiments, both T¹ and T² are absent.

In certain embodiments, each of T¹, T², T³, T⁴, T⁵, and T⁶ is independently absent or a branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In certain embodiments, each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In certain embodiments, each of T¹, T², T³, T⁴, T⁵, and T⁶ is independently absent or a branched or unbranched, saturated or unsaturated, hydrocarbon chain, having from 1 to 50 carbon atoms, or a salt thereof, wherein one or more (e.g. 1, 2, 3, or 4) of the carbon atoms in the hydrocarbon chain is optionally replaced by —O— or —NR^{X}—, and wherein R^{X} is hydrogen or (C₁-C₆)alkyl, and wherein the hydrocarbon chain, is optionally substituted with one or more (e.g. 1, 2, 3, or 4) substituents selected from halo, hydroxy, and oxo (═O).

In certain embodiments, each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

In certain embodiments, each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

In certain embodiments, at least one of T³, T⁴, T⁵, and T⁶ is:

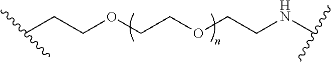

wherein:

n=1, 2, 3.

In certain embodiments, each of T³, T⁴, T⁵, and T⁶ is independently selected from the group consisting of:

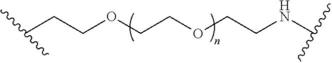

wherein:

n=1, 2, 3.

In certain embodiments, at least one of T¹ and T² is glycine

In certain embodiments, each of T¹ and T² is glycine.

In certain embodiments, B¹ is a trivalent group comprising 1 to 15 atoms and is covalently bonded to L¹, T¹, and T².

In certain embodiments, B¹ is a trivalent group comprising 1 to 10 atoms and is covalently bonded to L¹, T¹, and T².

In certain embodiments, B¹ comprises a (C₁-C₆)alkyl

In certain embodiments, B¹ comprises a C₃₋₈ cycloalkyl.

In certain embodiments, B¹ comprises a silyl group.

In certain embodiments, B¹ comprises a D- or L-amino acid.

In certain embodiments, B¹ comprises a saccharide.

In certain embodiments, B¹ comprises a phosphate group.

In certain embodiments, B¹ comprises a phosphonate group.

In certain embodiments, B¹ comprises an aryl.

In certain embodiments, B¹ comprises a phenyl ring.

In certain embodiments, B¹ is a phenyl ring.

In certain embodiments, B¹ is CH.

In certain embodiments, B¹ comprises a heteroaryl.

In certain embodiments, B¹ is:

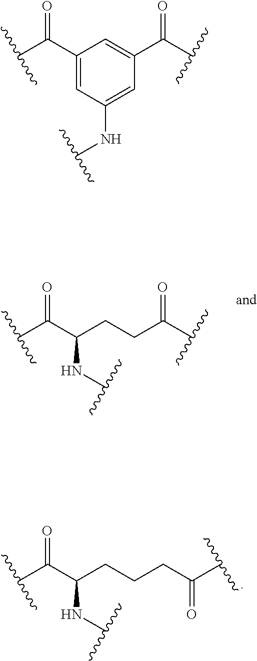 [SMILES: *Nc1cc(C(*)=O)cc(C(*)=O)c1 ; *N[C@H](CCC(*)=O)C(*)=O ; *N[C@H](CCCC(*)=O)C(*)=O]

In certain embodiments, B² is a trivalent group comprising 1 to 15 atoms and is covalently bonded to L¹, T¹, and T².

In certain embodiments, B² is a trivalent group comprising 1 to 10 atoms and is covalently bonded to L¹, T¹, and T².

In certain embodiments, B² comprises a (C₁-C₆)alkyl

In certain embodiments, B² comprises a C₃₋₈ cycloalkyl.

In certain embodiments, B² comprises a silyl group.

In certain embodiments, B² comprises a D- or L-amino acid.

In certain embodiments, B² comprises a saccharide.

In certain embodiments, B² comprises a phosphate group.

In certain embodiments, B² comprises a phosphonate group.

In certain embodiments, B² comprises an aryl.

In certain embodiments, B² comprises a phenyl ring.

In certain embodiments, B² is a phenyl ring.

In certain embodiments, B² is CH.

In certain embodiments, B² comprises a heteroaryl.

In certain embodiments, B² is selected from the group consisting of.

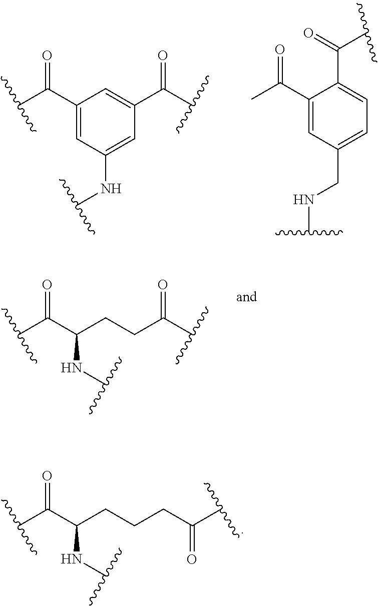 [SMILES: *Nc1cc(C(*)=O)cc(C(*)=O)c1 ; *NCc1ccc(C(*)=O)c(C(C)=O)c1 ; *N[C@H](CCC(*)=O)C(*)=O ; *N[C@H](CCCC(*)=O)C(*)=O]

In certain embodiments, B³ is a trivalent group comprising 1 to 15 atoms and is covalently bonded to L¹, T¹, and T².

In certain embodiments, B³ is a trivalent group comprising 1 to 10 atoms and is covalently bonded to L¹, T¹, and T².

In certain embodiments, B³ comprises a (C₁-C₆)alkyl

In certain embodiments, B³ comprises a C₃₋₈ cycloalkyl.

In certain embodiments, B³ comprises a silyl group.

In certain embodiments, B³ comprises a D- or L-amino acid.

In certain embodiments, B³ comprises a saccharide.

In certain embodiments, B³ comprises a phosphate group.

In certain embodiments, B³ comprises a phosphonate group.

In certain embodiments, B³ comprises an aryl.

In certain embodiments, B³ comprises a phenyl ring.

In certain embodiments, B³ is a phenyl ring.

In certain embodiments, B³ is CH.

In certain embodiments, B³ comprises a heteroaryl.

In certain embodiments, B³ is selected from the group consisting of:

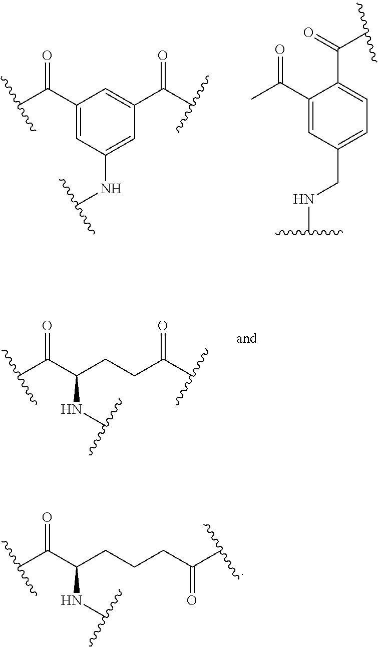 [SMILES: *Nc1cc(C(*)=O)cc(C(*)=O)c1 ; *NCc1ccc(C(*)=O)c(C(C)=O)c1 ; *N[C@H](CCC(*)=O)C(*)=O ; *N[C@H](CCCC(*)=O)C(*)=O]

In certain embodiments, L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In certain embodiments, L¹ is selected from the group consisting of:

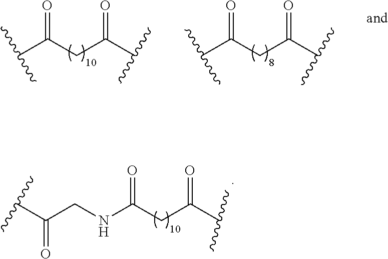

In certain embodiments, L¹ is connected to B¹ through a linkage selected from the group consisting of: —O—, —S—, —(C═O)—, —(C═O)—NH—, —NH—(C═O), —(C═O)—O—, —NH—(C═O)—NH—, or —NH—(SO₂)—.

In certain embodiments, L¹ is selected from the group consisting of:

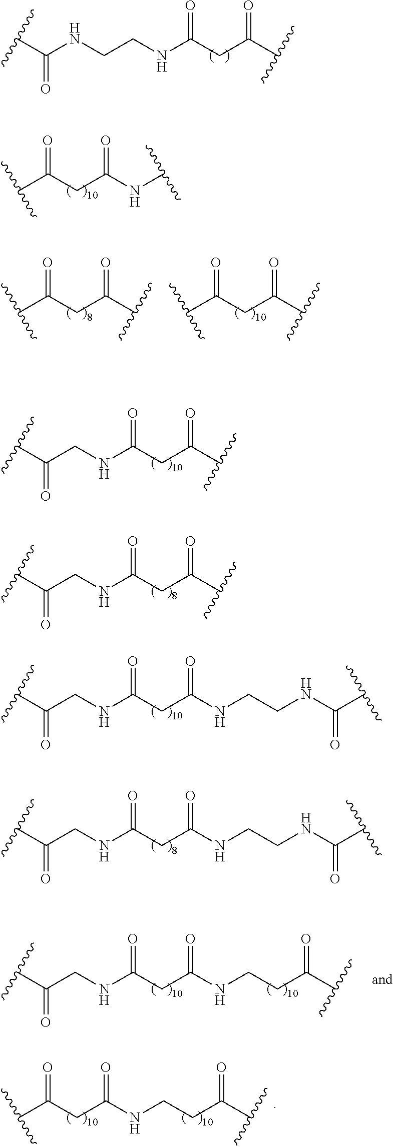

In certain embodiments, L² is connected to R² through —O—.

In certain embodiments, L² is C₁₋₄ alkylene-O— that is optionally substituted with hydroxy.

In certain embodiments, L² is connected to R² through —O—.

In certain embodiments, L² is absent.

In certain embodiments, the conjugate is selected from the group consisting of:

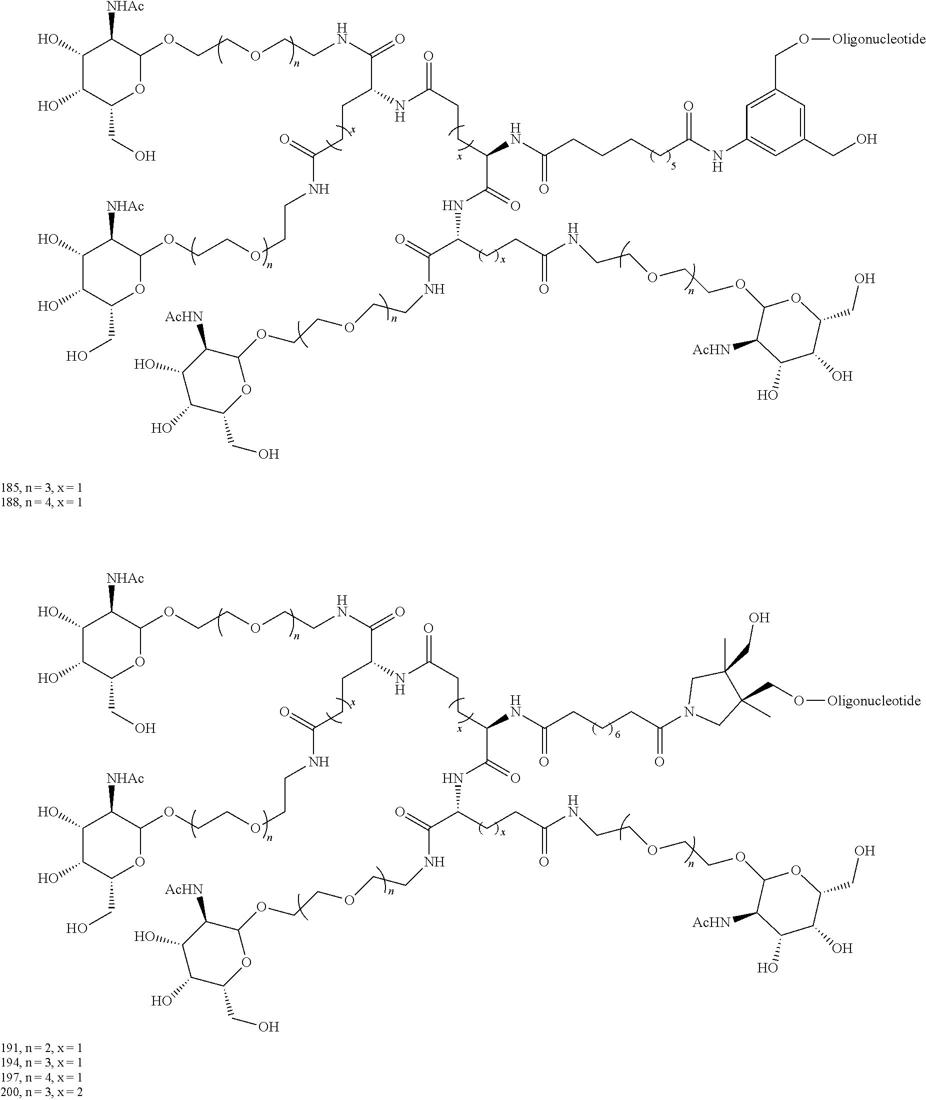

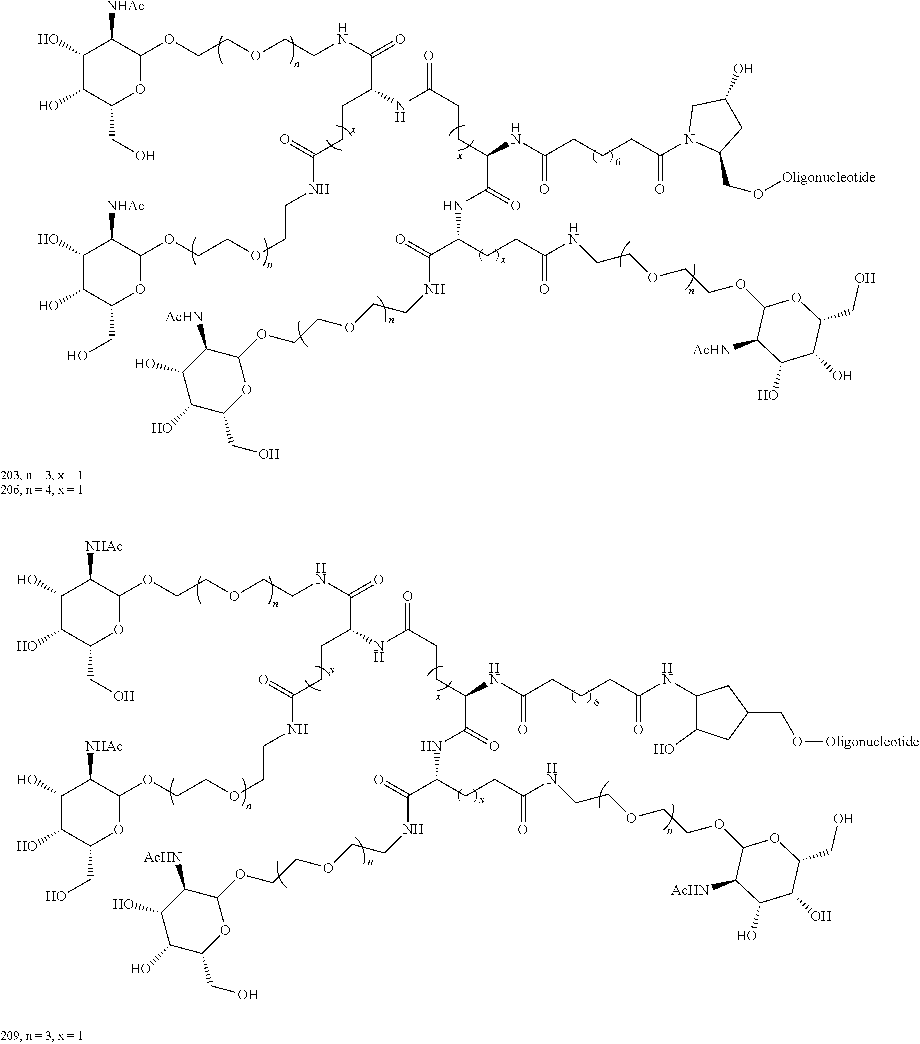

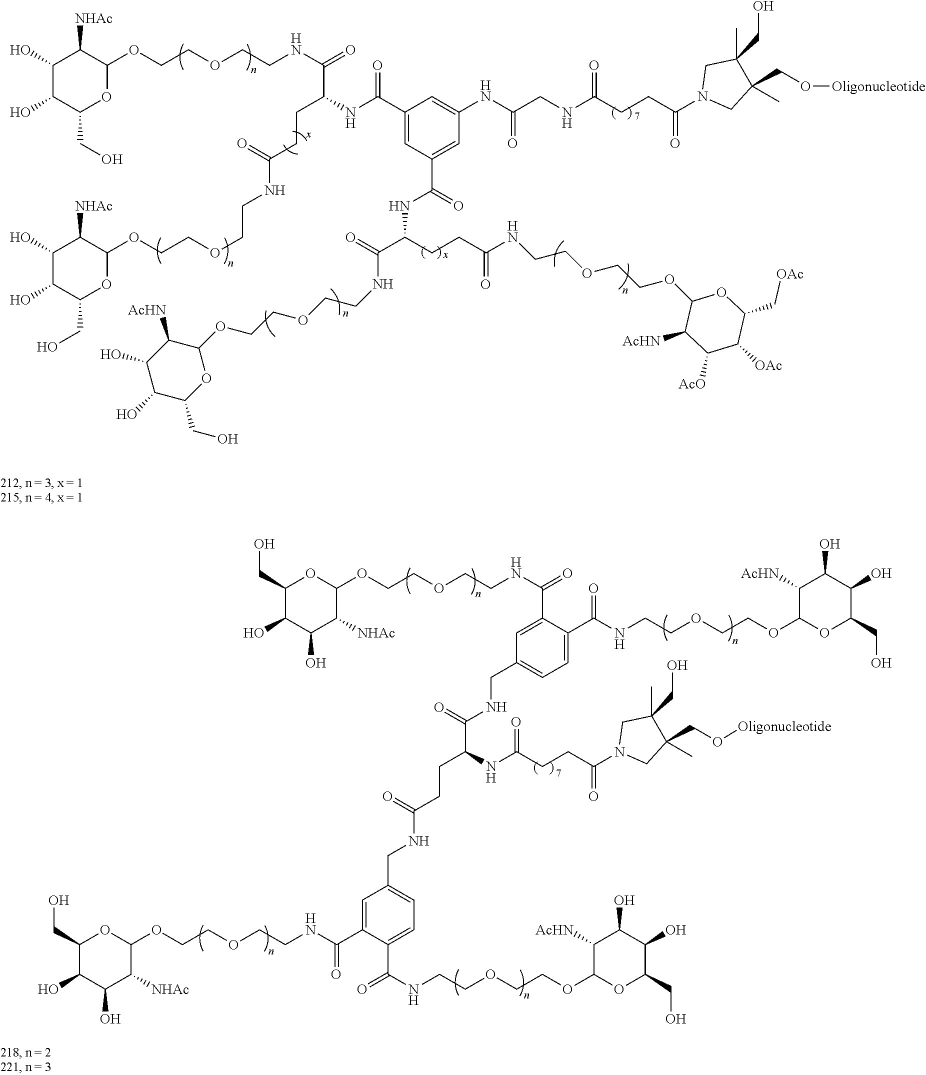

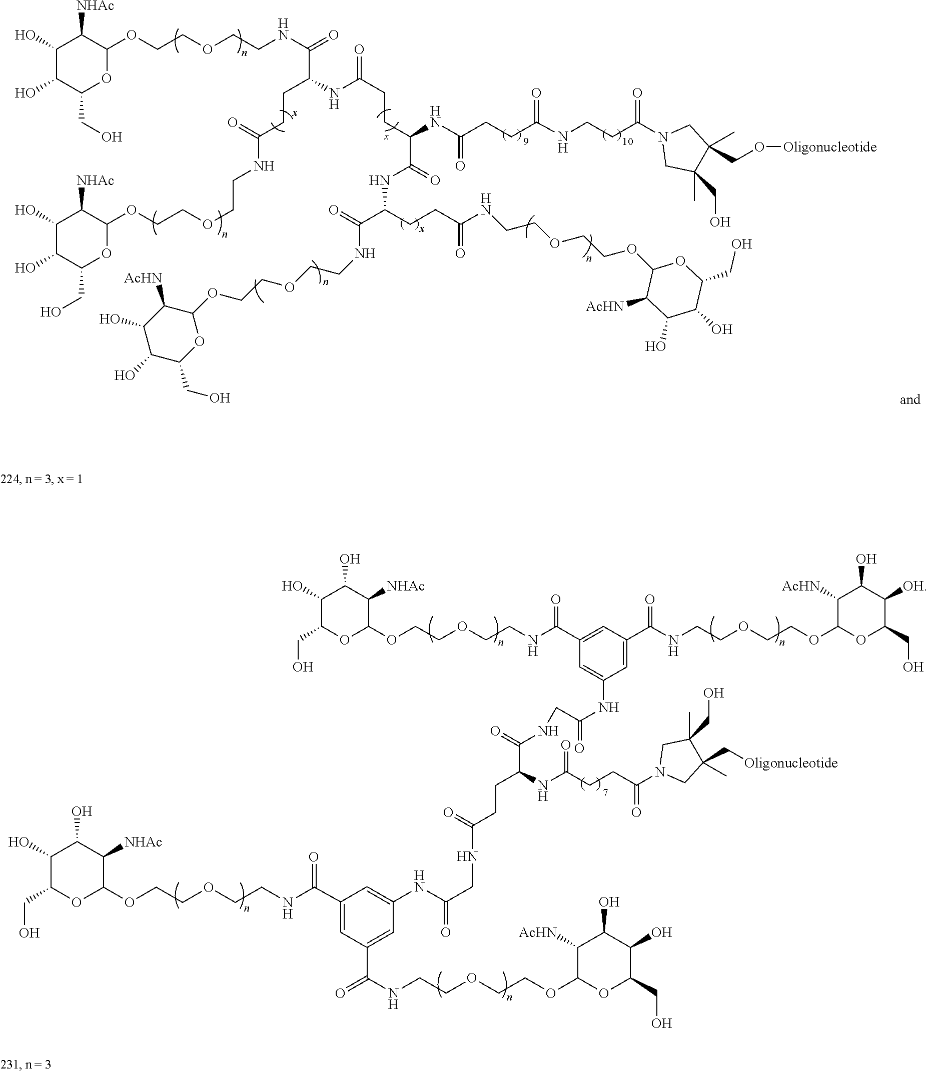

In certain embodiments, the conjugate is

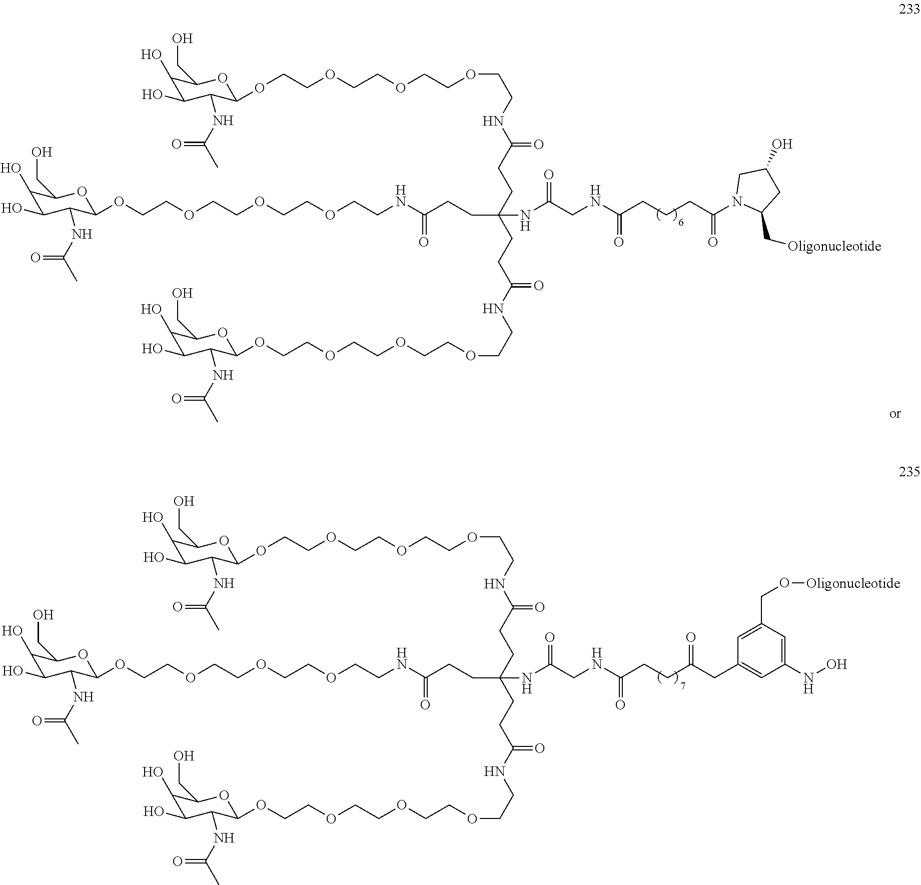

or a salt thereof.

In certain embodiments, the conjugate is conjugate of formula:

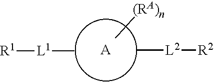

wherein the following definitions apply:

R¹ a is targeting ligand;

L¹ is absent or a linking group;

L² is absent or a linking group;

R² is a double stranded siRNA molecule;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen, or a protecting group; and

n is 0, 1, 2, 3, 4, 5, 6, 7, 8, 9, or 10;

or a salt thereof.

In certain embodiments,

R¹ a is targeting ligand;

L¹ is absent or a linking group;

L² is absent or a linking group;

R² is a double stranded siRNA molecule;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B} and C₁₋₈ alkyl that is optionally substituted with one or more groups independently selected from halo, hydroxy, and C₁₋₃ alkoxy;

R^{B} is hydrogen, or a protecting group; and

n is 0, 1, 2, 3, 4, 5, 6, 7, 8, 9, or 10.

In certain embodiments, R¹ is —C(H)₍₃₋ₚ₎(L³-saccharide)ₚ,

wherein each L³ is independently a linking group;

p is 1, 2, or 3; and

saccharide is a monosaccharide or disaccharide.

In certain embodiments, the saccharide is:

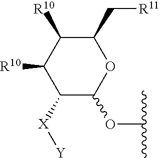

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

In certain embodiments, the saccharide is selected from the group consisting of:

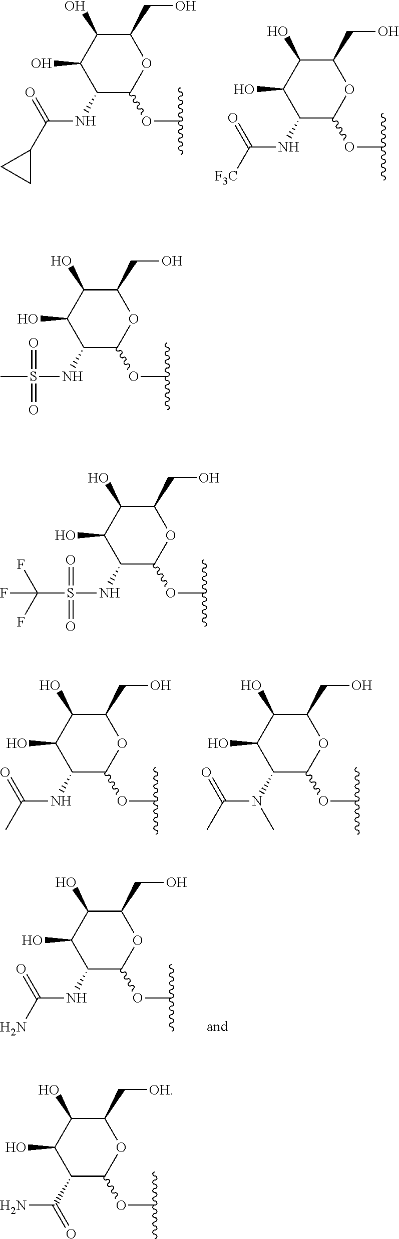

In certain embodiments, the saccharide is:

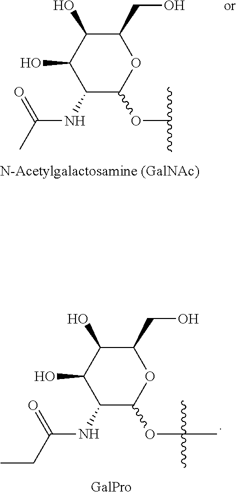 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In certain embodiments, each L³ is independently a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, each L³ is independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, L³ is:

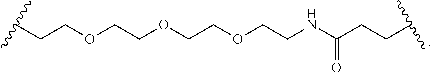 [SMILES: *CCOCCOCCOCCNC(=O)CC*]

In certain embodiments, R¹ is:

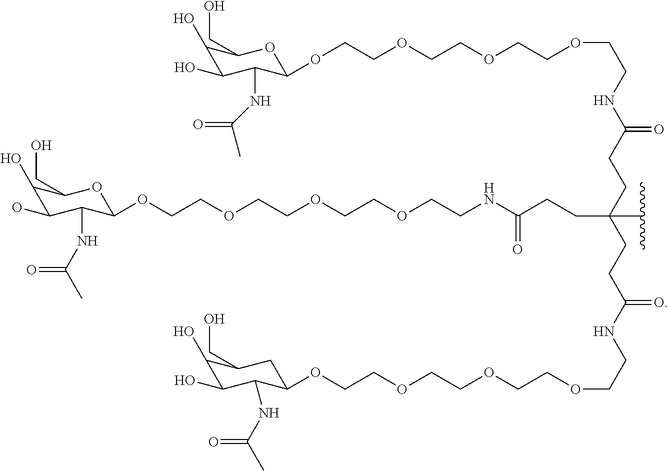 [SMILES: *C(CCC(=O)NCCOCCOCCOCCOC1CC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In certain embodiments, R¹ is:

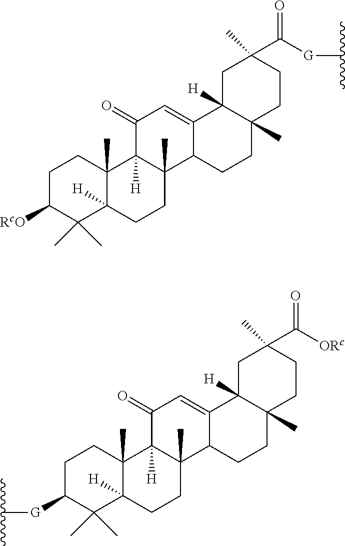

wherein:

G is —NH— or —O—;

R^{C} is hydrogen, (C₁-C₈)alkyl, (C₁-C₈)haloalkyl, (C₁-C₈)alkoxy, (C₁-C₆)alkanoyl, (C₃-C₂₀)cycloalkyl, (C₃-C₂₀)heterocycle, aryl, heteroaryl, monosaccharide, disaccharide or trisaccharide; and wherein the cycloalkyl, heterocyle, ary, heteroaryl and saccharide are optionally substituted with one or more groups independently selected from the group consisting of halo, carboxyl, hydroxyl, amino, (C₁-C₄)alkyl, (C₁-C₄)haloalkyl, (C₁-C₄)alkoxy and (C₁-C₄)haloalkoxy.

In certain embodiments, R^{C} is:

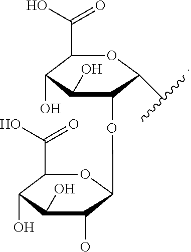

In certain embodiments, R¹ is:

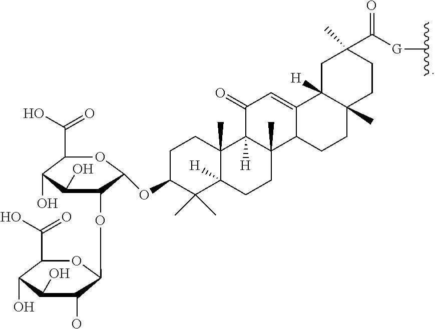

In certain embodiments, R^{C} is:

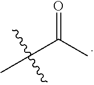 [SMILES: *C(C)=O]

In certain embodiments, G is —NH—.

In certain embodiments, R¹ is:

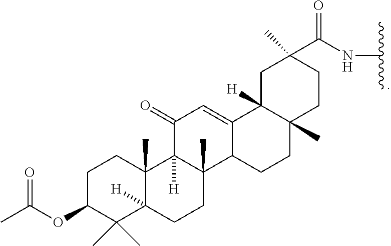

In certain embodiments, R¹ is:

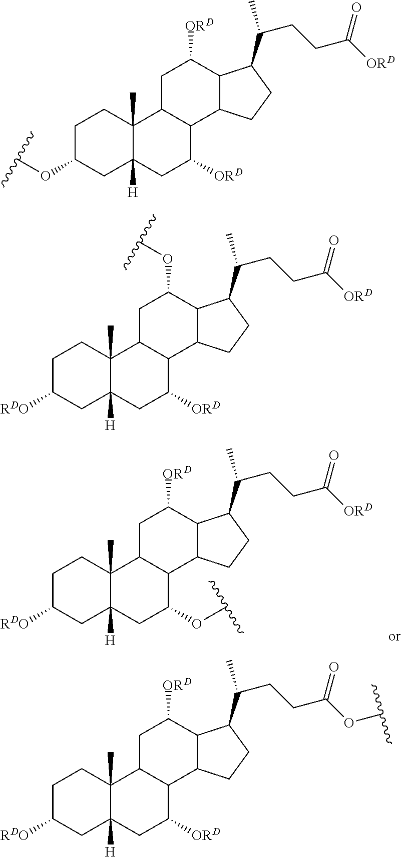

wherein each R^{D} is independently selected from the group consisting of hydrogen, (C₁—C₆)alkyl, (C₉-C₂₀)alkylsilyl, (R^{W})₃Si—, (C₂-C₆)alkenyl, tetrahydropyranyl, (C₁-C₆)alkanoyl, benzoyl, aryl(C₁-C₃)alkyl, TMTr (Trimethoxytrityl), DMTr (Dimethoxytrityl), MMTr (Monomethoxytrityl), and Tr (Trityl); and

each R^{W} is independently selected from the group consisting of (C₁-C₄)alkyl and aryl.

In certain embodiments, L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, L¹ and L² are independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, L¹ and L² are independently, a divalent, branched or unbranched, saturated or unsaturated, hydrocarbon chain, having from 1 to 14 carbon atoms, wherein one or more (e.g. 1, 2, 3, or 4) of the carbon atoms in the hydrocarbon chain is optionally replaced —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, L¹ is connected to R¹ through —NH—, —O—, —S—, —(C═O)—, —(C═O)—NH—, —NH—(C═O)—, —(C═O)—O—, —NH—(C═O)—NH—, or —NH—(SO₂)—.

In certain embodiments, L² is connected to R² through —O—.

In certain embodiments, L¹ is selected from the group consisting of:

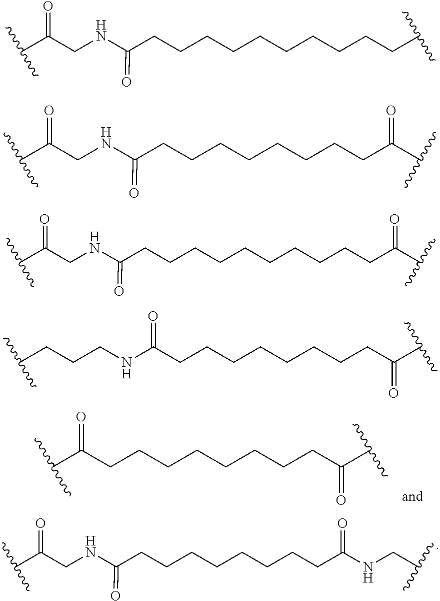

In certain embodiments, L² is —CH₂—O— or —CH₂—CH₂—O—.

In certain embodiments, the conjugate is a conjugate of the following formula:

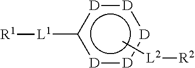

wherein:
each D is independently selected from the group consisting of

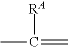

### and —N═.

In certain embodiments, the conjugate is a conjugate of the following formula

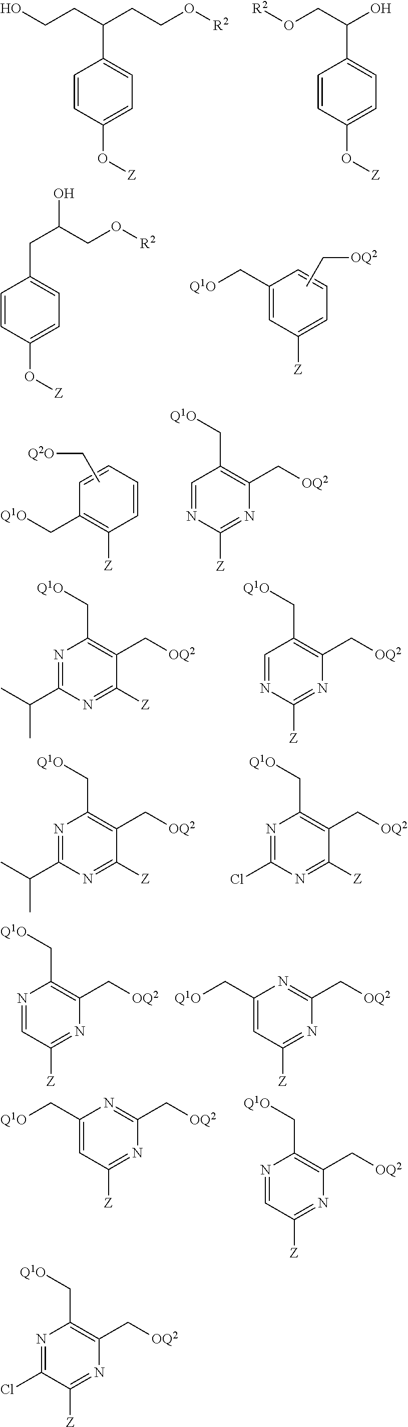

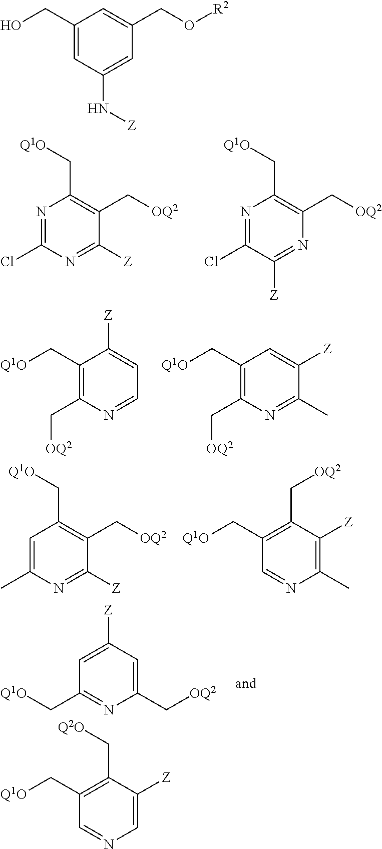

wherein:

Q¹ is hydrogen and Q² is R²; or Q¹ is R² and Q² is hydrogen; and

Z is -L¹-R¹.

In certain embodiments, the conjugate is a conjugate of the following formula

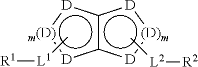

wherein:

each D is independently selected from the group consisting of

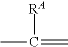

### and —N═; and

each m is independently 1 or 2.

In certain embodiments, the conjugate is selected from the group consisting of:

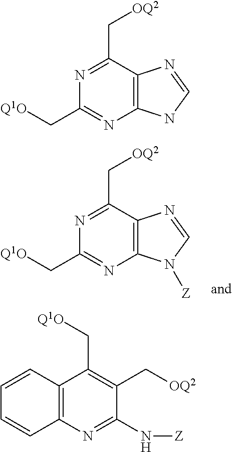

wherein:

Q¹ is hydrogen and Q² is R²; or Q¹ is R² and Q² is hydrogen; and

Z is -L¹-R¹.

In certain embodiments, the conjugate is a conjugate of the following formula

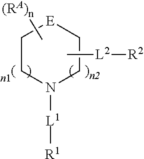

wherein:

E is —O— or —CH₂—;

n is selected from the group consisting of 0, 1, 2, 3, and 4; and

n1 and n2 are each independently selected from the group consisting of 0, 1, 2, and 3.

In certain embodiments, the conjugate is selected from the group consisting of:

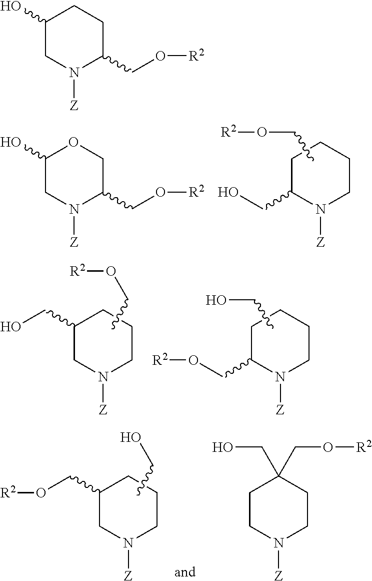

wherein: Z is -L¹-R¹.

In certain embodiments, the -A-L²-R² moiety is:

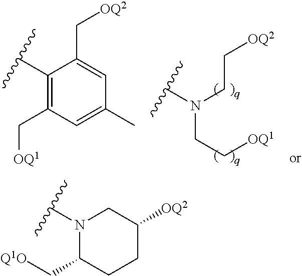

wherein:

Q¹ is hydrogen and Q² is R²; or Q¹ is R² and Q² is hydrogen; and

each q is independently 0, 1, 2, 3, 4 or 5.

In certain embodiments, the conjugate selected from the group consisting of:

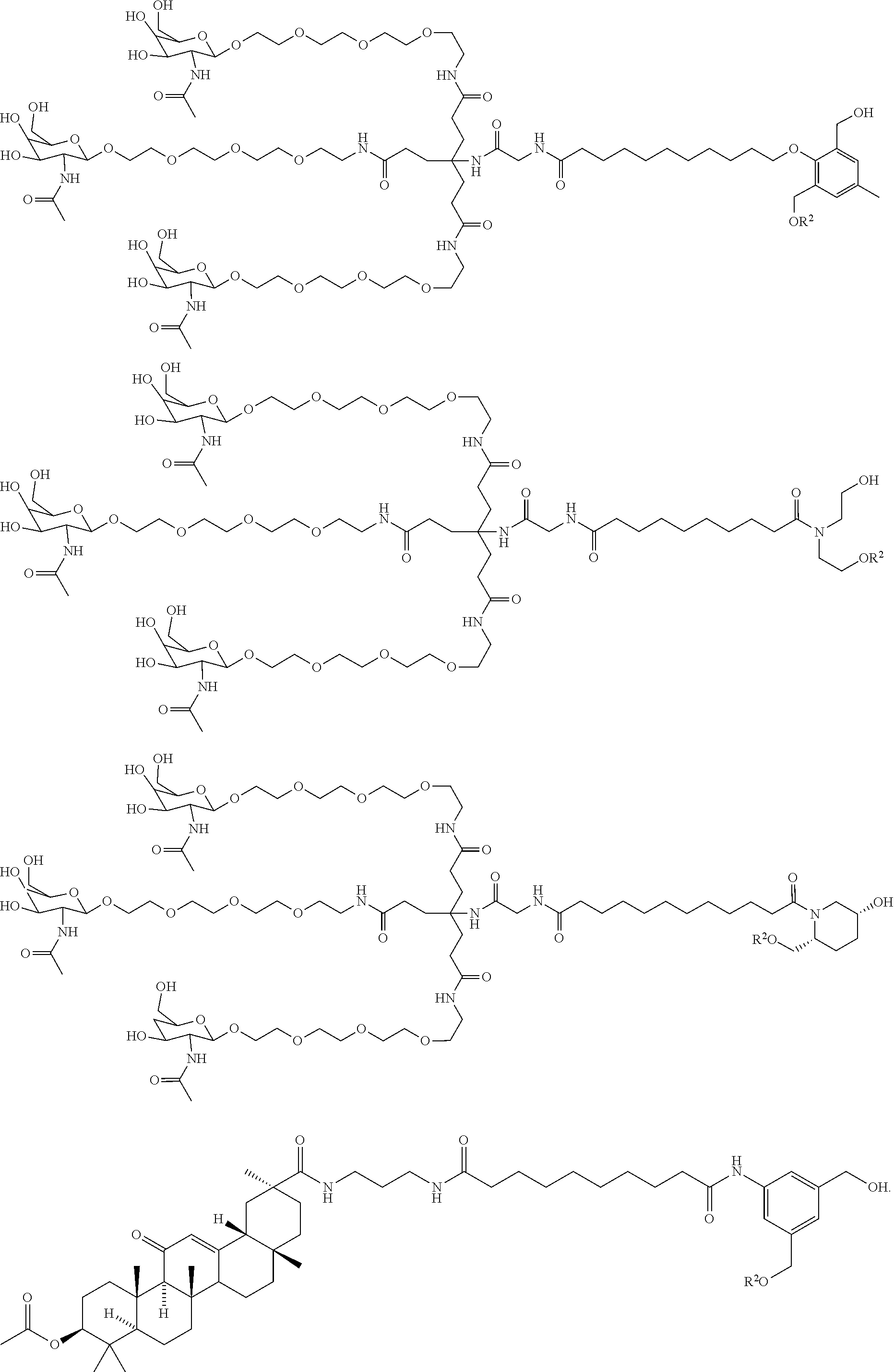

In certain embodiments, R¹ is selected from the group consisting of

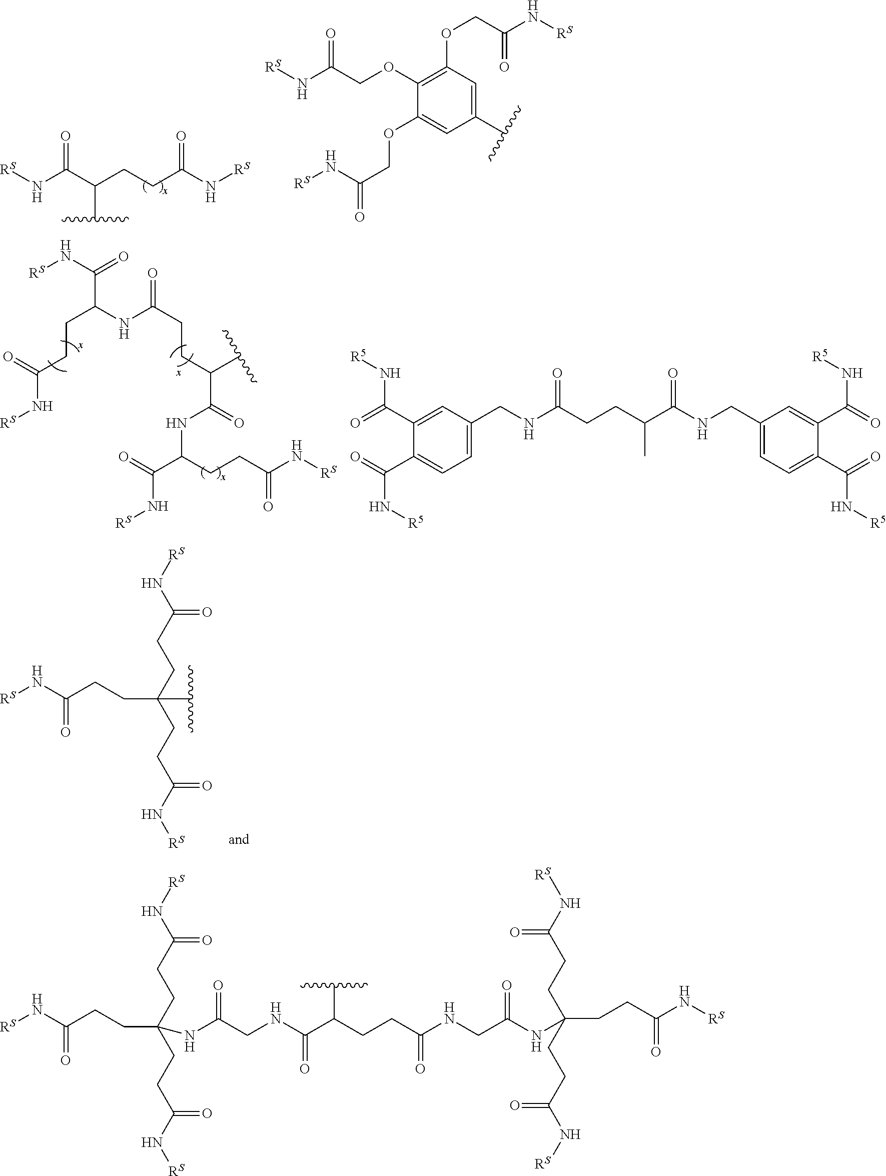

wherein:

R^{S} is

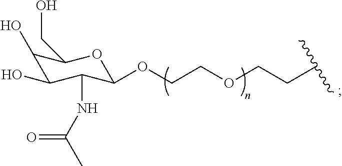

n is 2, 3, or 4; and

x is 1 or 2.

In certain embodiments, L¹ is selected from the group consisting of:

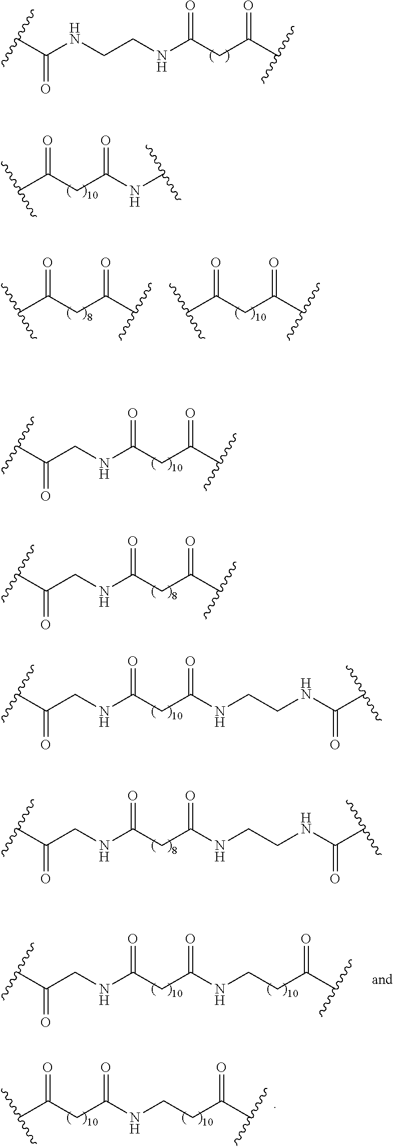

In certain embodiments, A is absent, phenyl, pyrrolidinyl, or cyclopentyl.

In certain embodiments, L² is C₁₋₄ alkylene-O— that is optionally substituted with hydroxy.

In certain embodiments, L² is —CH₂O—, —CH₂CH₂O—, or —CH(OH)CH₂O—.

In certain embodiments, each R^{A} is independently hydroxy or C₁₋₈ alkyl that is optionally substituted with hydroxyl.

In certain embodiments, each R^{A} is independently selected from the group consisting of hydroxy, methyl and —CH₂OH.

In certain embodiments, the conjugate is a conjugate of the following formula:

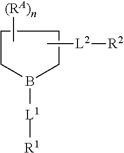

wherein:

B is —N— or —CH—;

L² is C₁₋₄ alkylene-O— that is optionally substituted with hydroxyl or halo; and

n is 0, 1, 2, 3, 4, 5, 6, or 7.

In certain embodiments, the conjugate is selected from the group consisting of:

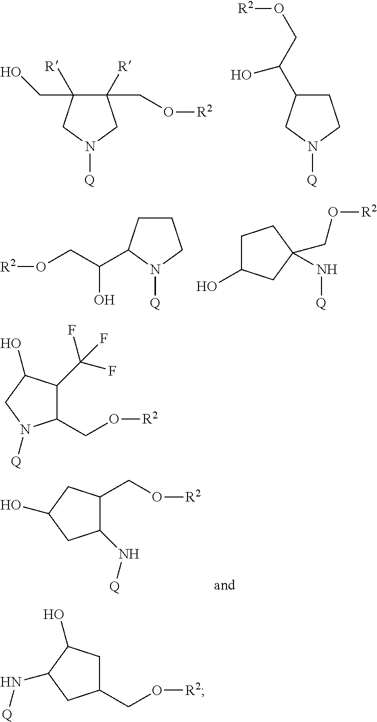

wherein Q is -L¹-R¹; and

R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

In certain embodiments, the conjugate is selected from the group consisting of:

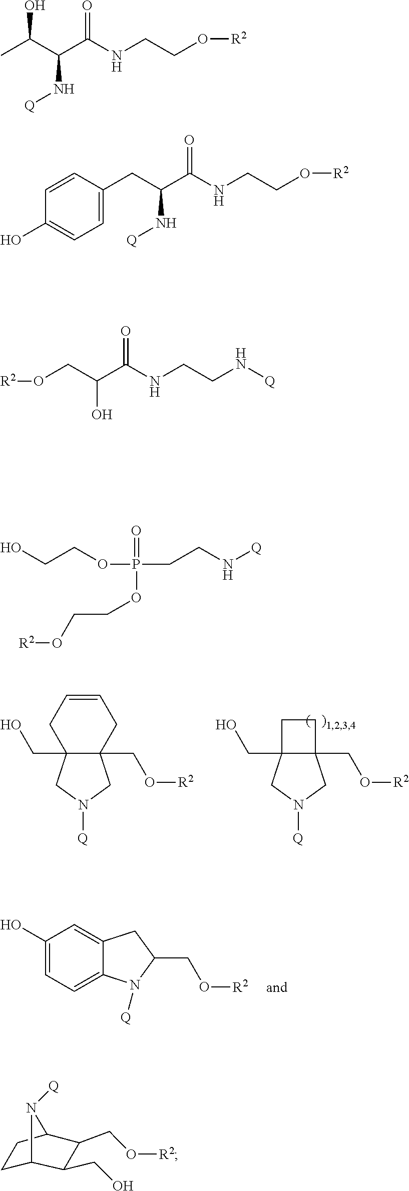

wherein Q is -L¹-R¹.

In certain embodiments, the conjugate is selected from the group consisting of:

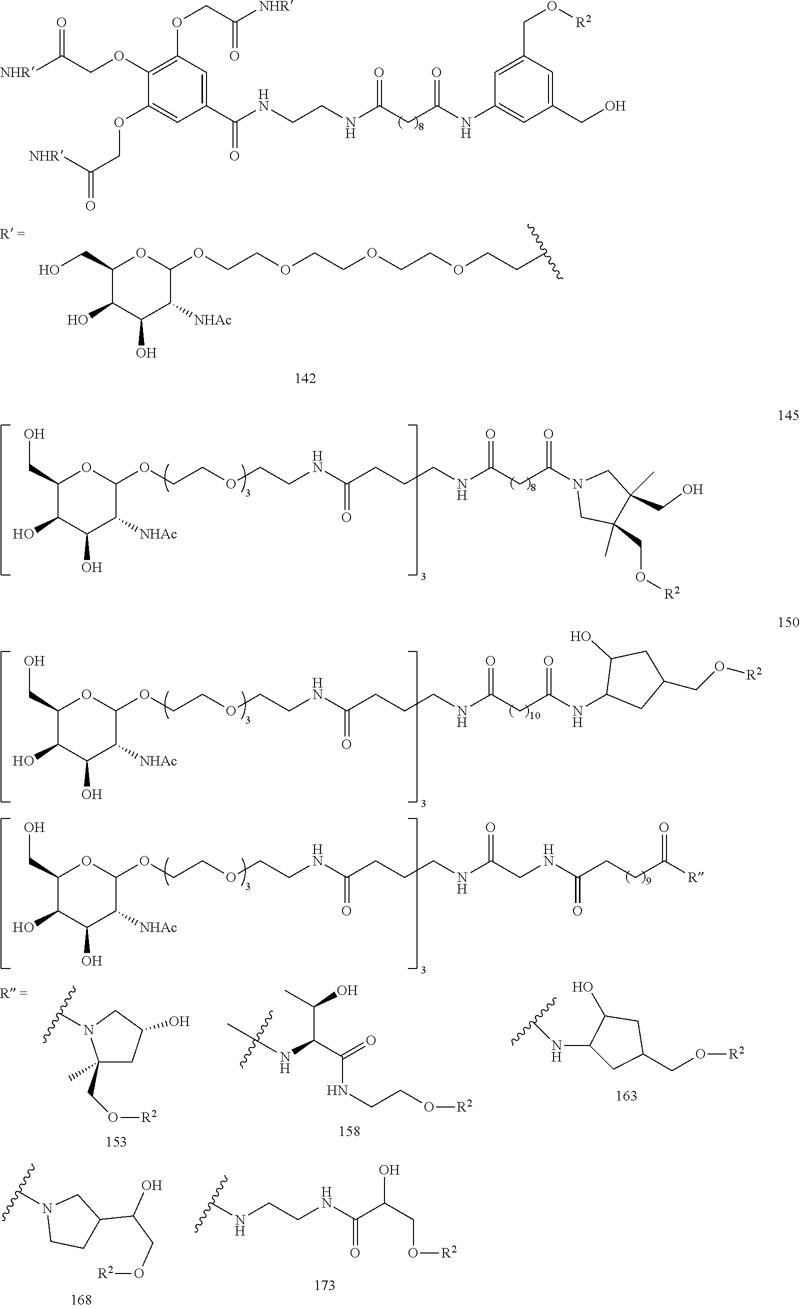

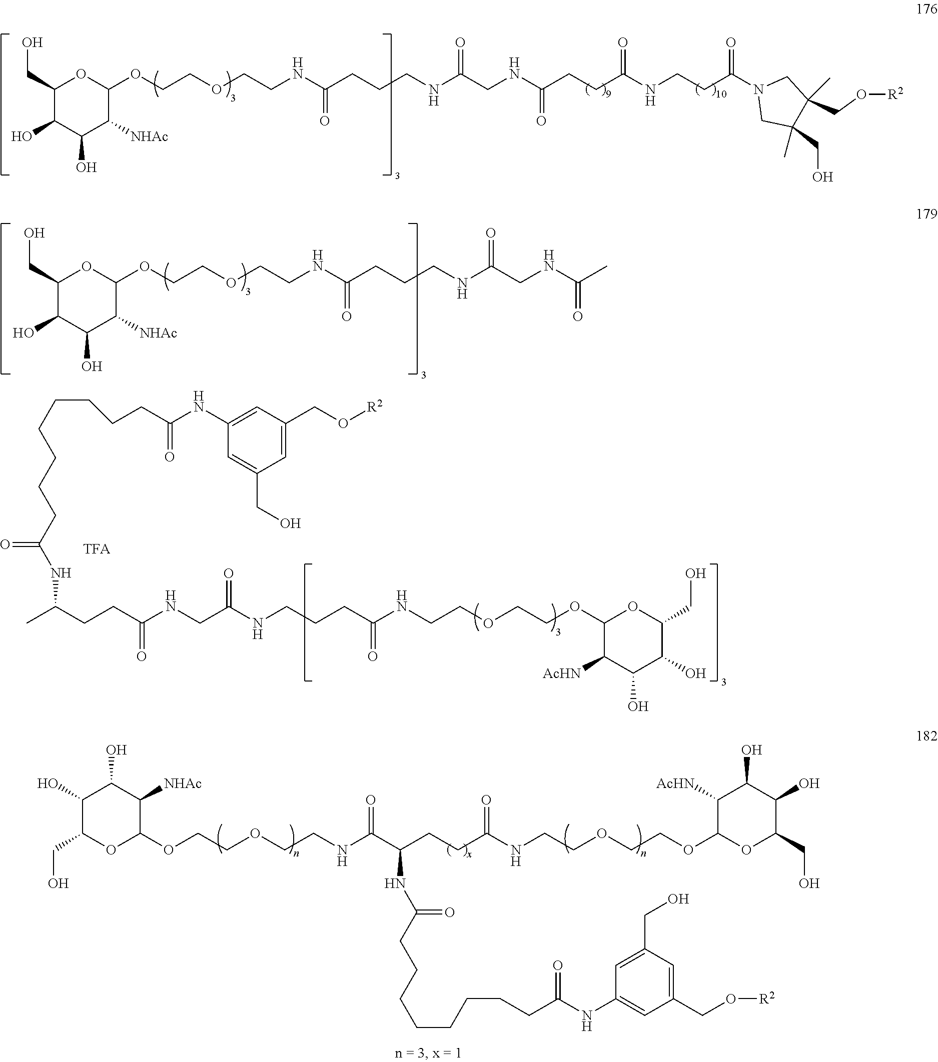

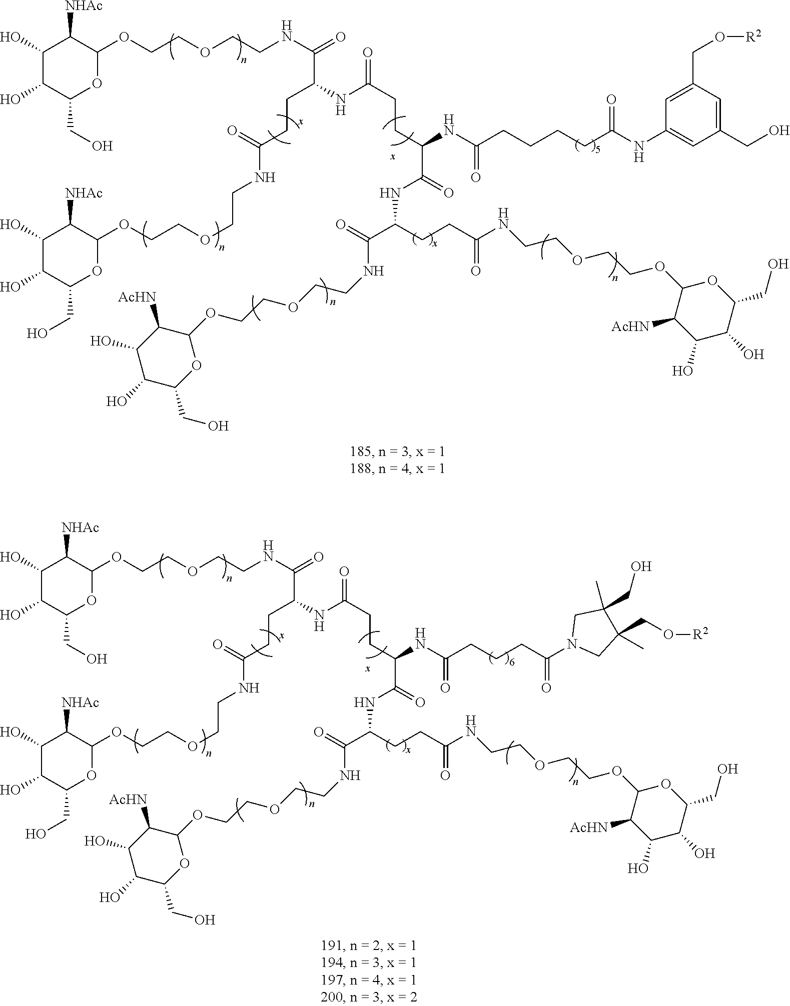

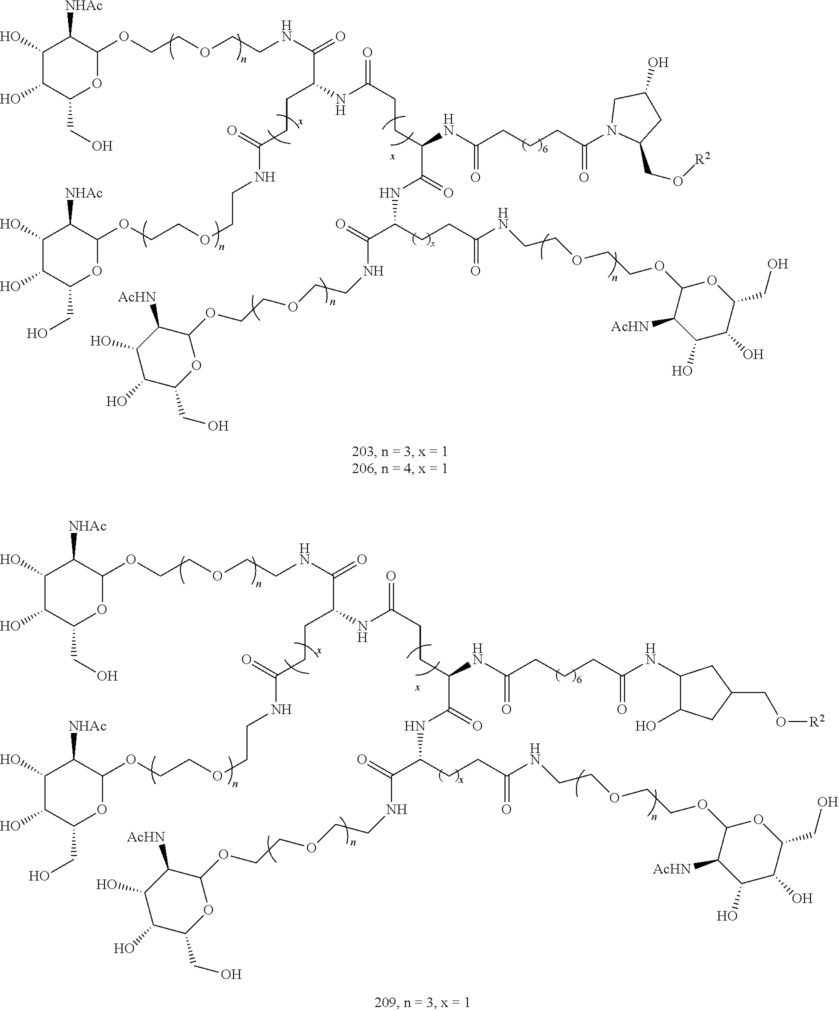

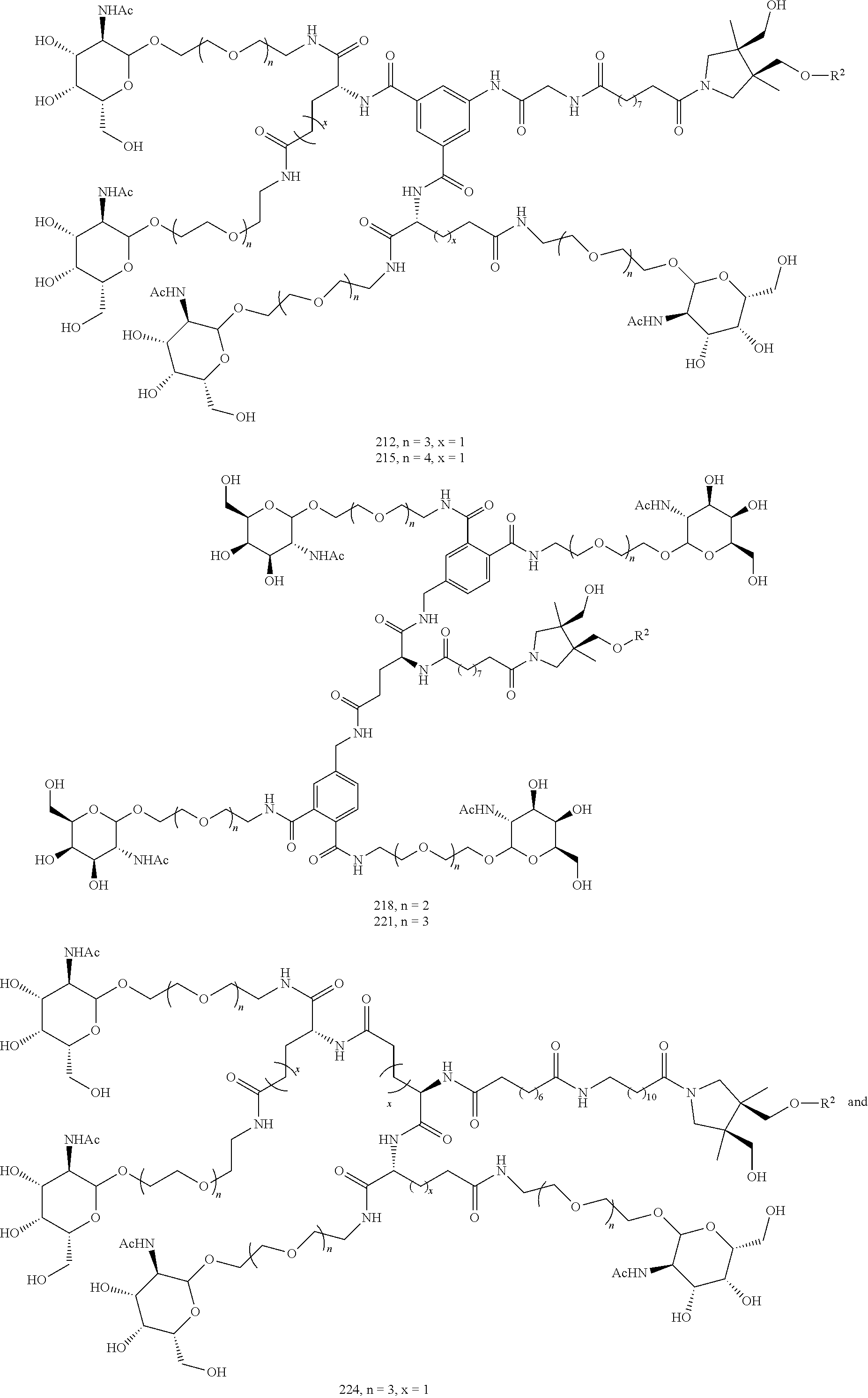

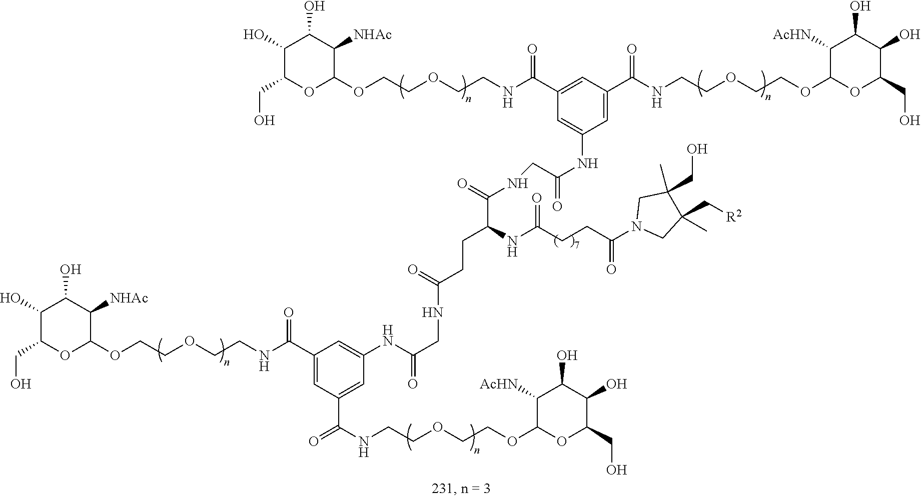

and pharmaceutically acceptable salts thereof, wherein R² is a double stranded siRNA molecule.

In certain embodiments, the conjugate is a conjugate of the following formula:

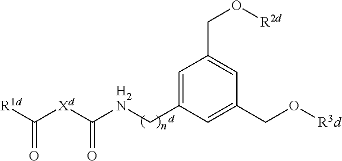

wherein:

R^{1d} is selected from:

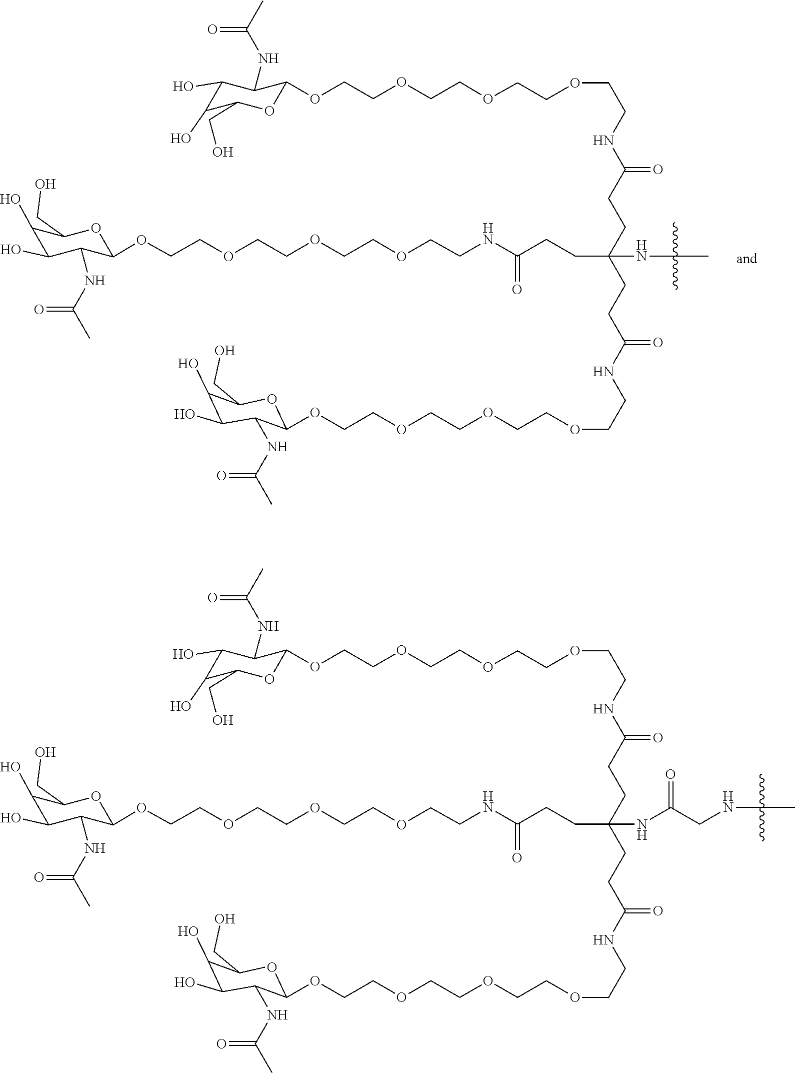 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

X^{d} is C₂₋₁₀ alkylene;

n^{d} is 0 or 1;

R^{2d} is a double stranded siRNA molecule; and

R^{3d} is H, or a protecting group.

In certain embodiments R^{1d} is:

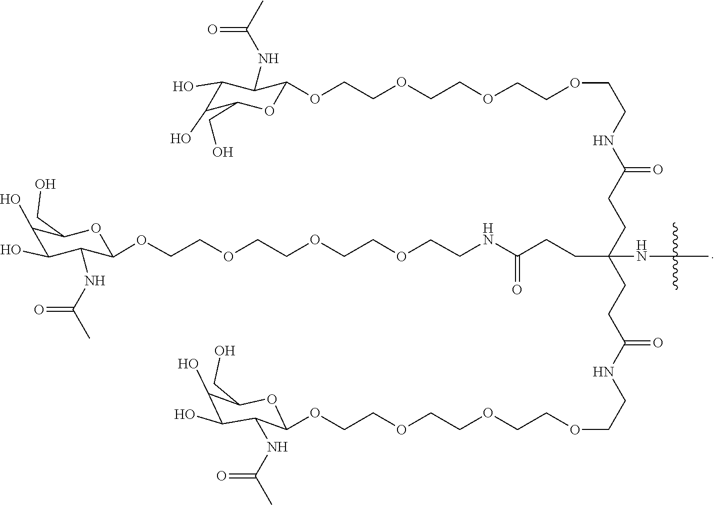 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In certain embodiments, R^{1d} is:

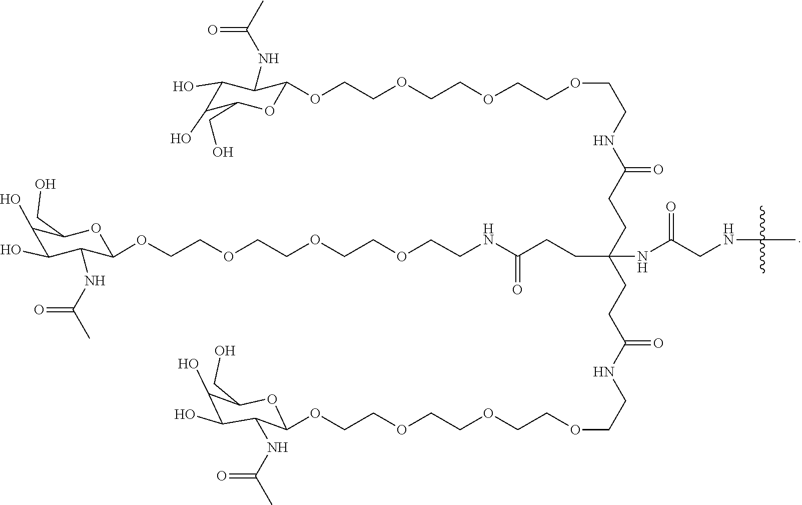 [SMILES: *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In certain embodiments, X^{d} is C₈alkylene.

In certain embodiments, n^{d} is 0.

In certain embodiments, R^{3d} is H.

In certain embodiments, the conjugate is selected from the group consisting of:

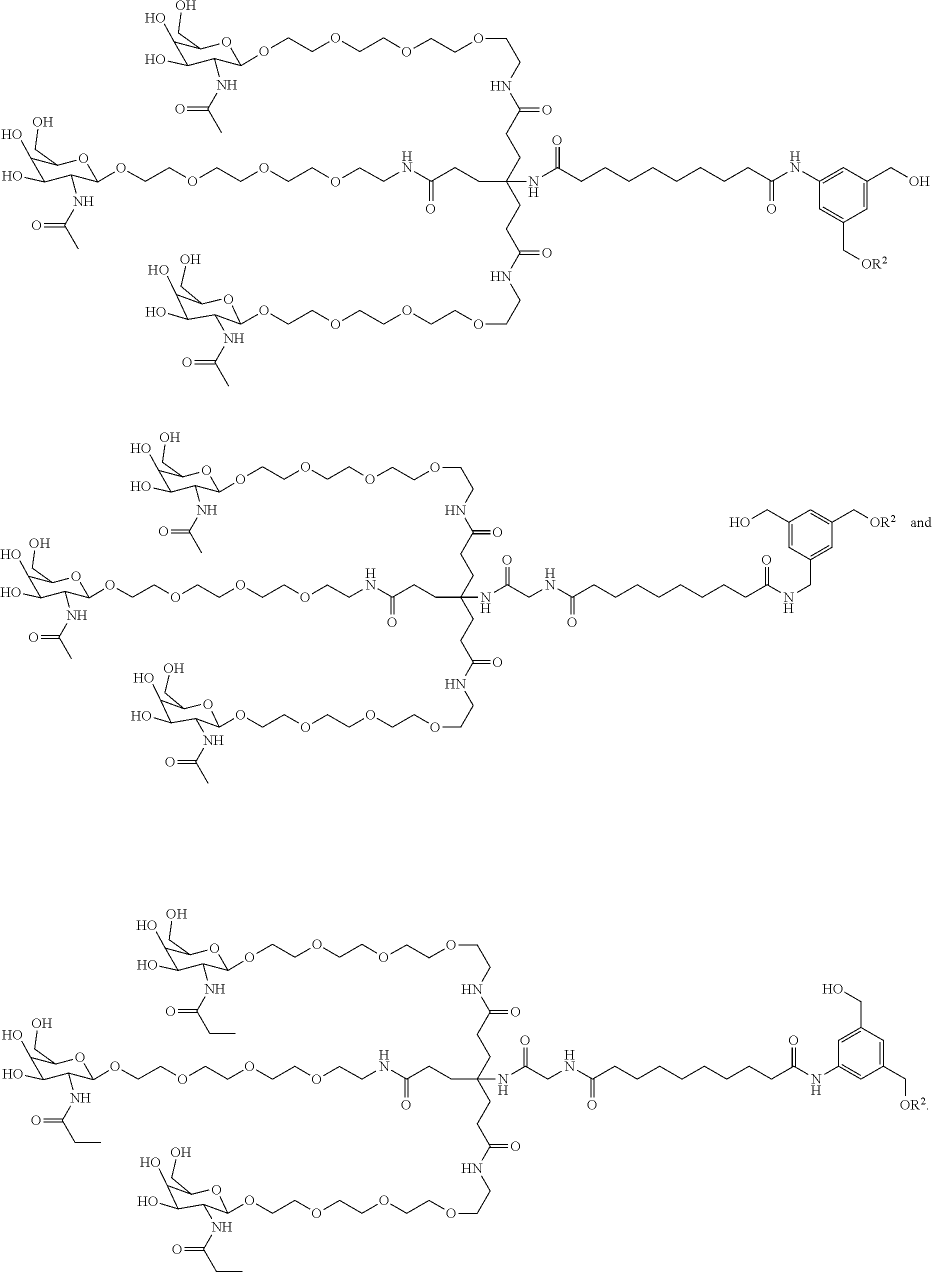

In certain embodiments, the conjugate is a conjugate of the following formula

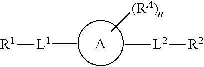

wherein the following definitions apply:

R¹ is H or a synthetic activating group;

L¹ is absent or a linking group;

L² is absent or a linking group;

R² is a double stranded siRNA molecule;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen, or a protecting group; and

n is 0, 1, 2, 3, 4, 5, 6, 7, 8, 9, or 10;

or a salt thereof.

In certain embodiments, the conjugate is a conjugate of the following formula

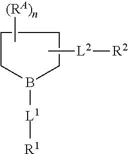

wherein:

B is —N— or —CH—;

L² is C₁₋₄ alkylene-O— that is optionally substituted with hydroxyl or halo; and

n is 0, 1, 2, 3, 4, 5, 6, or 7.

In certain embodiments, the conjugate is selected from the group consisting of:

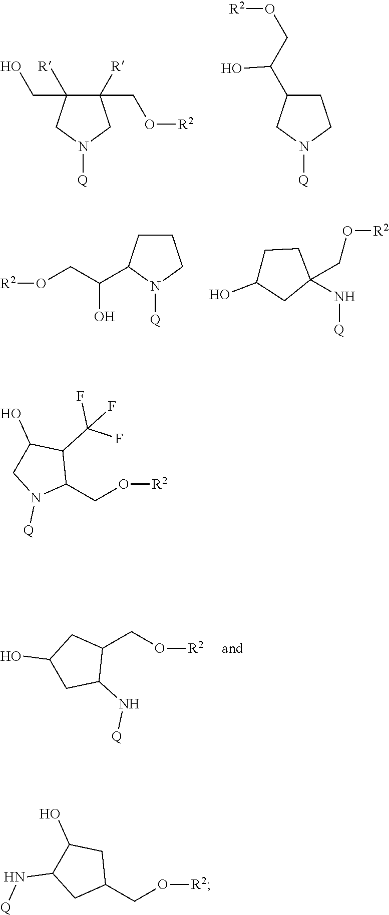

wherein:

Q is -L¹-R¹; and

R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

In certain embodiments, the conjugate is selected from the group consisting of:

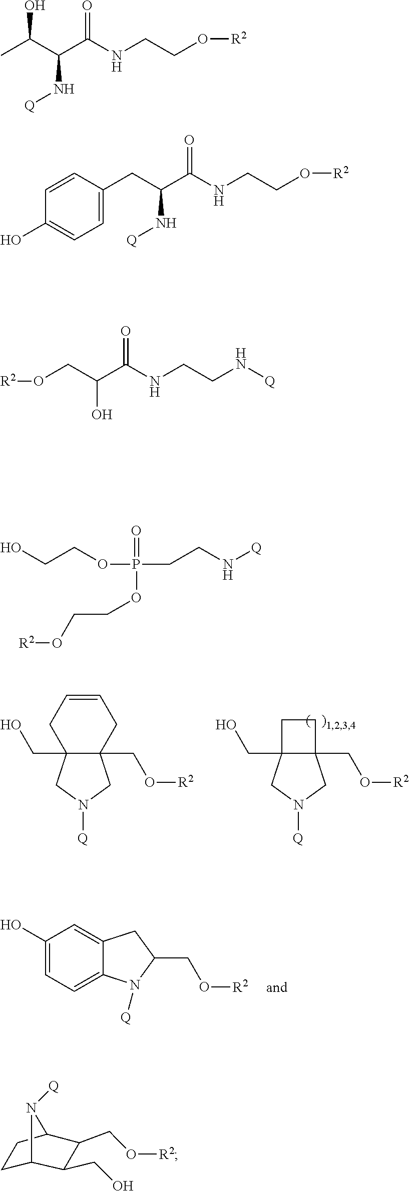

wherein: Q is -L¹-R¹.

In certain embodiments, R¹ is H or a synthetic activating group derivable from DCC, HOBt, EDC, BOP, PyBOP or HBTU.

In certain embodiments, R¹ is a synthetic activating group derivable from DCC, HOBt, EDC, BOP, PyBOP or HBTU.

In certain embodiments, L¹ is a divalent, branched or unbranched, saturated or unsaturated, hydrocarbon chain, having from 5 to 20 carbon atoms, wherein one or more (e.g. 1, 2, 3, or 4) of the carbon atoms in the hydrocarbon chain is optionally replaced —O—, —NH—, —NH—C(═O)—, —C(═O)—NH— or —S—.

In certain embodiments, the conjugate is a conjugate of the following formula:

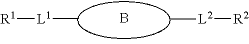

wherein the following definitions apply:

R¹ a is targeting ligand;

L¹ is absent or a linking group;

L² is absent or a linking group;

R² is a double stranded siRNA molecule;

B is divalent and is selected from the group consisting of:

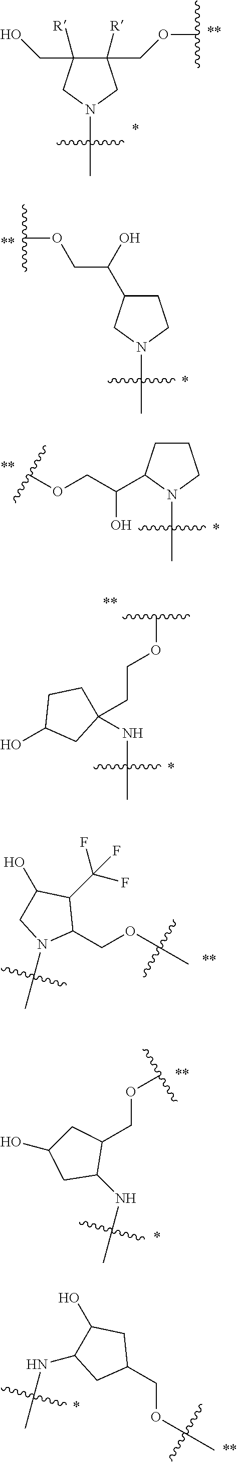

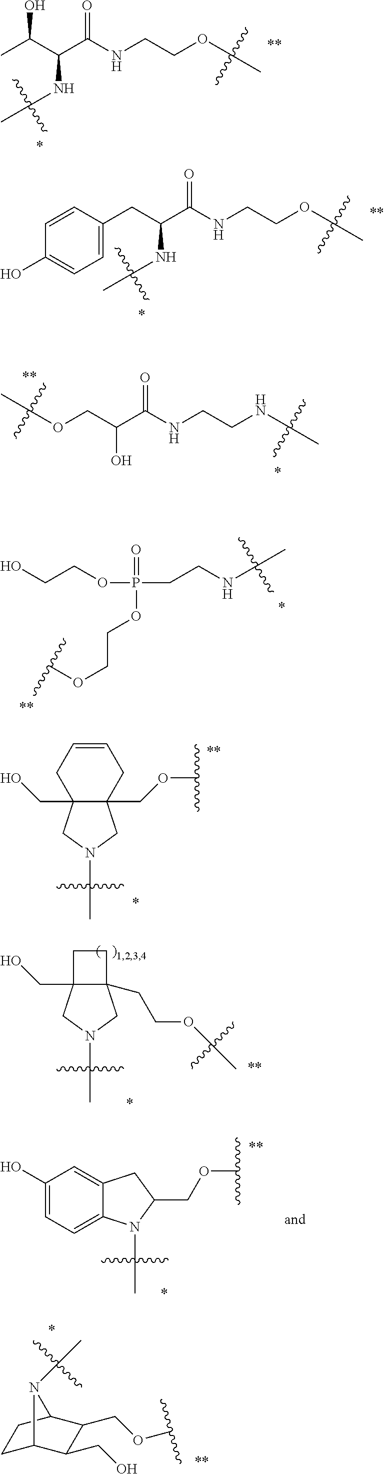

wherein:

each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;

the valence marked with * is attached to L¹ or is attached to R¹ if L¹ is absent; and

the valence marked with ** is attached to L² or is attached to R² if L² is absent;

or a salt thereof.

In certain embodiments, the targeting ligand R¹ comprises 2-8 saccharides.

In certain embodiments, the targeting ligand R¹ comprises 2-4 saccharides.

In certain embodiments, the targeting ligand R¹ comprises 3-8 saccharides.

In certain embodiments, the targeting ligand R¹ comprises 3-6 saccharides.

In certain embodiments, the targeting ligand R¹ comprises 3-4 saccharides.

In certain embodiments, the targeting ligand R¹ comprises 3 saccharides.

In certain embodiments, the targeting ligand R¹ comprises 4 saccharides.

In certain embodiments, the targeting moiety R¹ has the following formula:

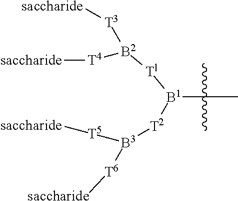

wherein:

B¹ is a trivalent group comprising about 1 to about 20 atoms and is covalently bonded to L¹, T¹, and T².

B² is a trivalent group comprising about 1 to about 20 atoms and is covalently bonded to T¹, T³, and T⁴;

B³ is a trivalent group comprising about 1 to about 20 atoms and is covalently bonded to T², T⁵, and T⁶;

T¹ is absent or a linking group;

T² is absent or a linking group;

T³ is absent or a linking group;

T⁴ is absent or a linking group;

T⁵ is absent or a linking group; and

T⁶ is absent or a linking group.

In certain embodiments, each saccharide is independently selected from:

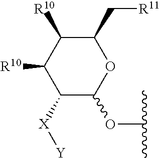

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

In certain embodiments, each the saccharide is independently selected from the group consisting of:

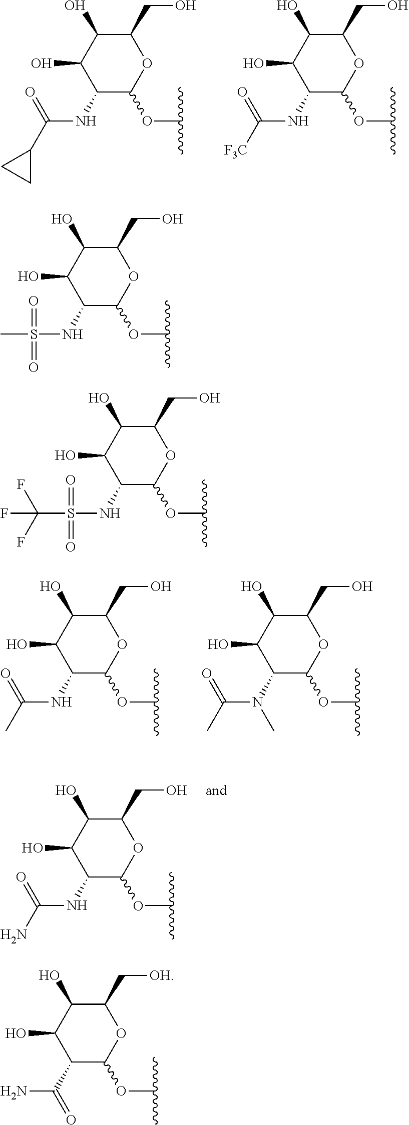

In certain embodiments, each saccharide is independently:

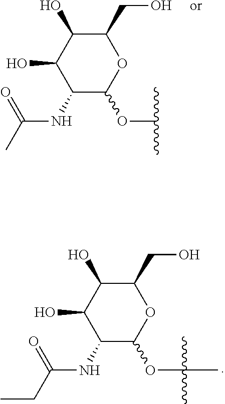

In certain embodiments, one of T¹ and T² is absent.

In certain embodiments, both T¹ and T² are absent.

In certain embodiments, each of T¹, T², T³, T⁴, T⁵, and T⁶ is independently absent or a branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In certain embodiments, each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In certain embodiments, each of T¹, T², T³, T⁴, T⁵, and T⁶ is independently absent or a branched or unbranched, saturated or unsaturated, hydrocarbon chain, having from 1 to 50 carbon atoms, or a salt thereof, wherein one or more (e.g. 1, 2, 3, or 4) of the carbon atoms in the hydrocarbon chain is optionally replaced by —O— or —NR^{X}—, and wherein R^{X} is hydrogen or (C₁-C₆)alkyl, and wherein the hydrocarbon chain, is optionally substituted with one or more (e.g. 1, 2, 3, or 4) substituents selected from halo, hydroxy, and oxo (═O).

In certain embodiments, each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

In certain embodiments, each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

In certain embodiments, at least one of T³, T⁴, T⁵, and T⁶ is:

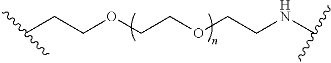

wherein:

n=1, 2, 3.

In certain embodiments, each of T³, T⁴, T⁵, and T⁶ is independently selected from the group consisting of:

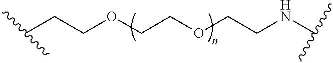

wherein:

n=1, 2, 3.

In certain embodiments, at least one of T¹ and T² is glycine.

In certain embodiments, each of T¹ and T² is glycine.

In certain embodiments, B¹ is a trivalent group comprising 1 to 15 atoms and is covalently bonded to L¹, T¹, and T².

In certain embodiments, B¹ is a trivalent group comprising 1 to 10 atoms and is covalently bonded to L¹, T¹, and T².

In certain embodiments, B¹ comprises a (C₁-C₆)alkyl

In certain embodiments, B¹ comprises a C₃₋₈ cycloalkyl.

In certain embodiments, B¹ comprises a silyl group.

In certain embodiments, B¹ comprises a D- or L-amino acid.

In certain embodiments, B¹ comprises a saccharide.

In certain embodiments, B¹ comprises a phosphate group.

In certain embodiments, B¹ comprises a phosphonate group.

In certain embodiments, B¹ comprises an aryl.

In certain embodiments, B¹ comprises a phenyl ring.

In certain embodiments, B¹ is a phenyl ring.

In certain embodiments, B¹ is CH.

In certain embodiments, B¹ comprises a heteroaryl.

In certain embodiments, B¹ is selected from

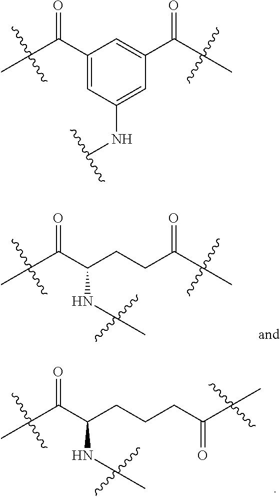 [SMILES: *Nc1cc(C(*)=O)cc(C(*)=O)c1 ; *N[C@@H](CCC(*)=O)C(*)=O ; *N[C@H](CCCC(*)=O)C(*)=O]

In certain embodiments, B² is a trivalent group comprising 1 to 15 atoms and is covalently bonded to L¹, T¹, and T².

In certain embodiments, B² is a trivalent group comprising 1 to 10 atoms and is covalently bonded to L¹, T¹, and T².

In certain embodiments, B² comprises a (C₁-C₆)alkyl.

In certain embodiments, B² comprises a C₃₋₈ cycloalkyl.

In certain embodiments, B² comprises a silyl group.

In certain embodiments, B² comprises a D- or L-amino acid.

In certain embodiments, B² comprises a saccharide.

In certain embodiments, B² comprises a phosphate group.

In certain embodiments, B² comprises a phosphonate group.

In certain embodiments, B² comprises an aryl.

In certain embodiments, B² comprises a phenyl ring.

In certain embodiments, B² is a phenyl ring.

In certain embodiments, B² is CH.

In certain embodiments, B² comprises a heteroaryl.

In certain embodiments, B² is selected from the group consisting of

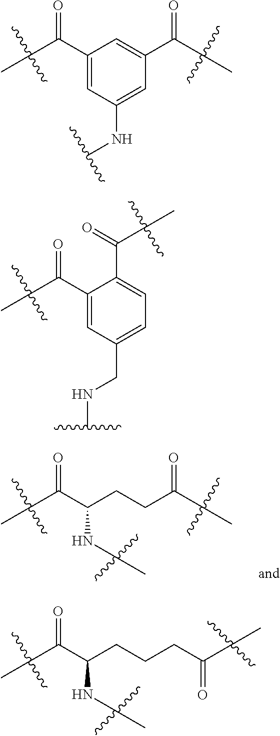

In certain embodiments, B³ is a trivalent group comprising 1 to 15 atoms and is covalently bonded to L¹, T¹, and T².

In certain embodiments, B³ is a trivalent group comprising 1 to 10 atoms and is covalently bonded to L¹, T¹, and T².

In certain embodiments, B³ comprises a (C₁-C₆)alkyl.

In certain embodiments, B³ comprises a C₃₋₈ cycloalkyl.

In certain embodiments, B³ comprises a silyl group.

In certain embodiments, B³ comprises a D- or L-amino acid.

In certain embodiments, B³ comprises a saccharide.

In certain embodiments, B³ comprises a phosphate group.

In certain embodiments, B³ comprises a phosphonate group.

In certain embodiments, B³ comprises an aryl.

In certain embodiments, B³ comprises a phenyl ring.

In certain embodiments, B³ is a phenyl ring.

In certain embodiments, B³ is CH.

In certain embodiments, B³ comprises a heteroaryl.

In certain embodiments, B³ is selected from the group consisting of

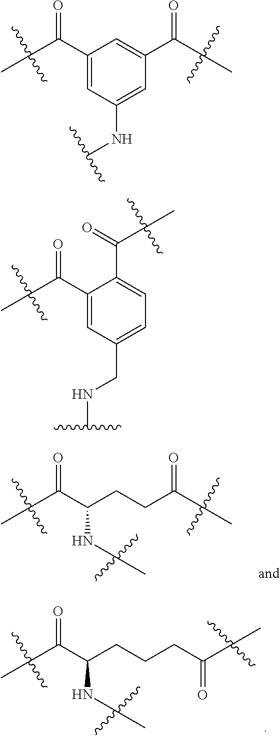

In certain embodiments, L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In certain embodiments, L¹ is selected from the group consisting of:

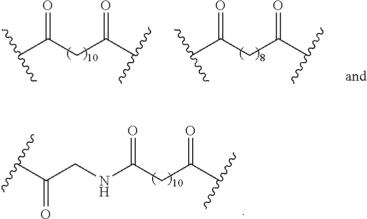

In certain embodiments, L¹ is connected to B¹ through a linkage selected from the group consisting of: —O—, —S—, —(C═O)—, —(C═O)—NH—, —NH—(C═O), —(C═O)—O—, —NH—(C═O)—NH—, or —NH—(SO₂)—.

In certain embodiments, L¹ is selected from the group consisting of:

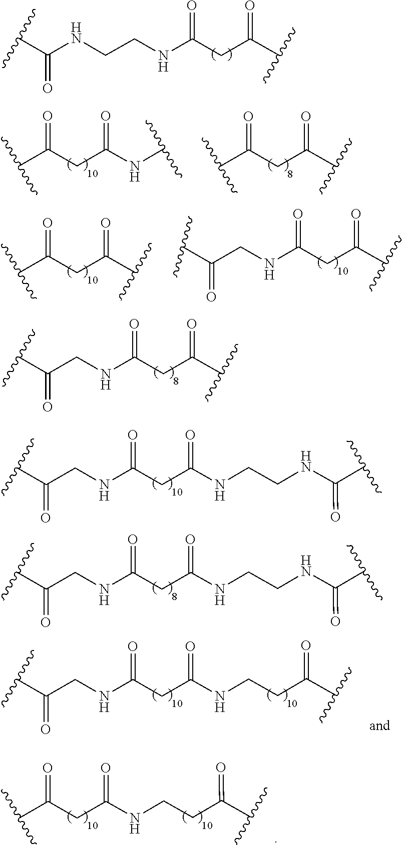

In certain embodiments, L² is connected to R² through —O—.

In certain embodiments, L² is C₁₋₄ alkylene-O— that is optionally substituted with hydroxy.

In certain embodiments, L² is connected to R² through —O—.

In certain embodiments, L² is absent.

In certain embodiments, the conjugate is selected from the group consisting of:

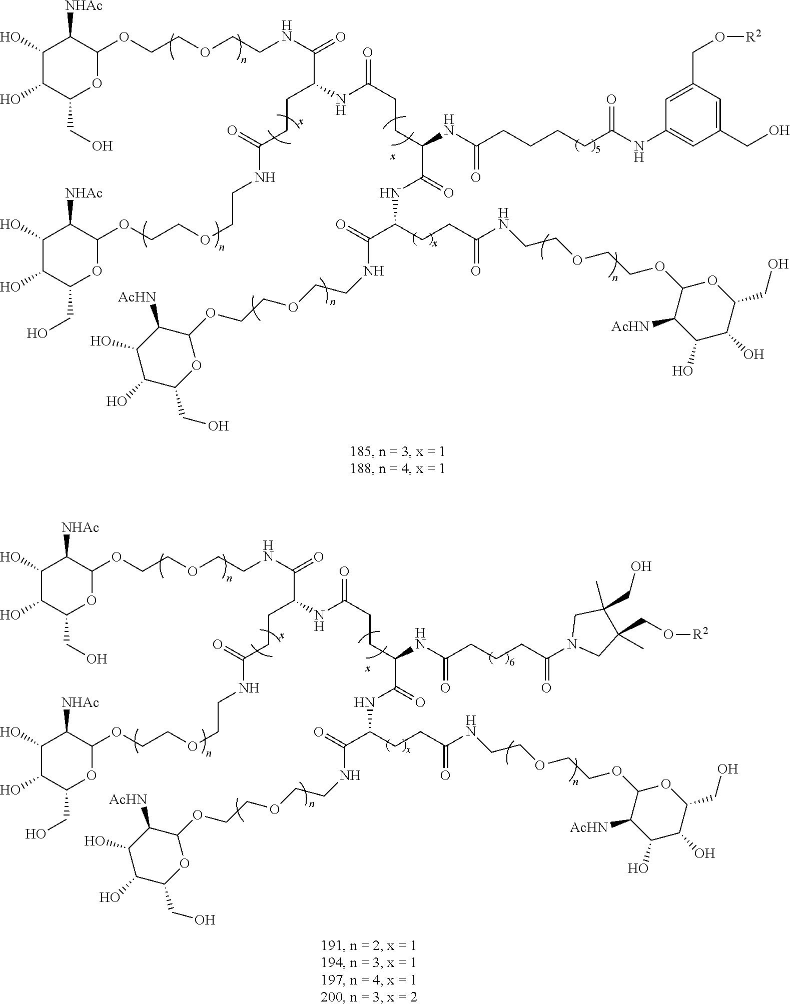

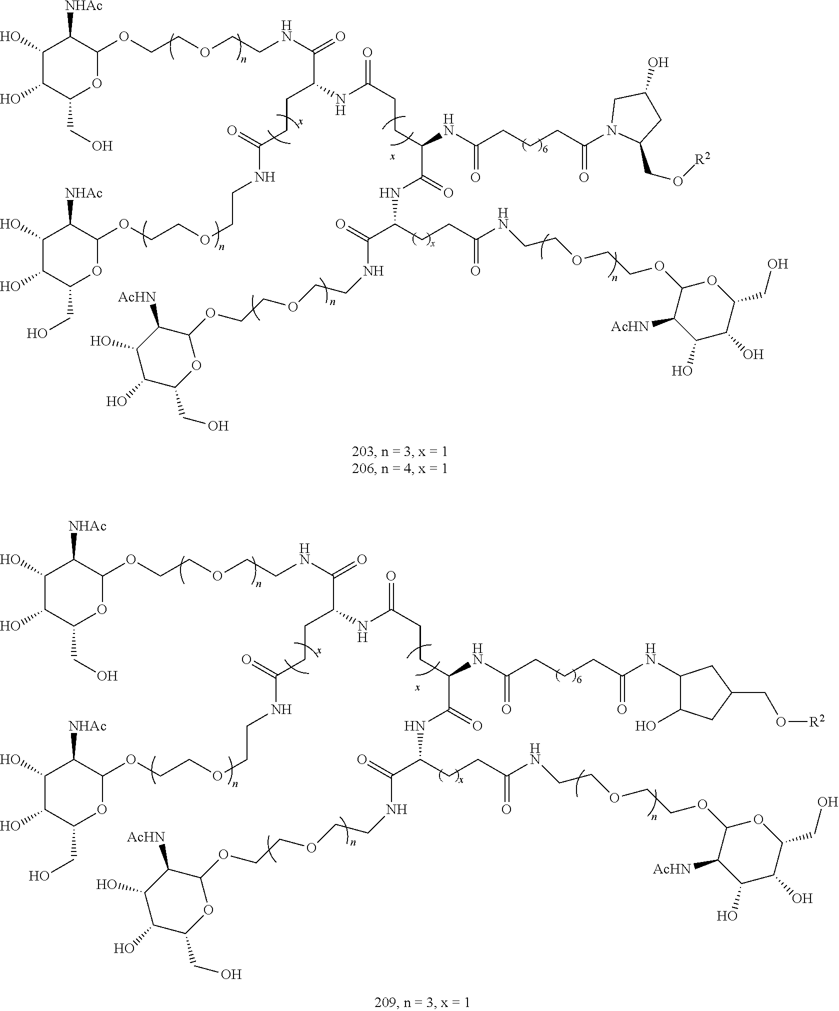

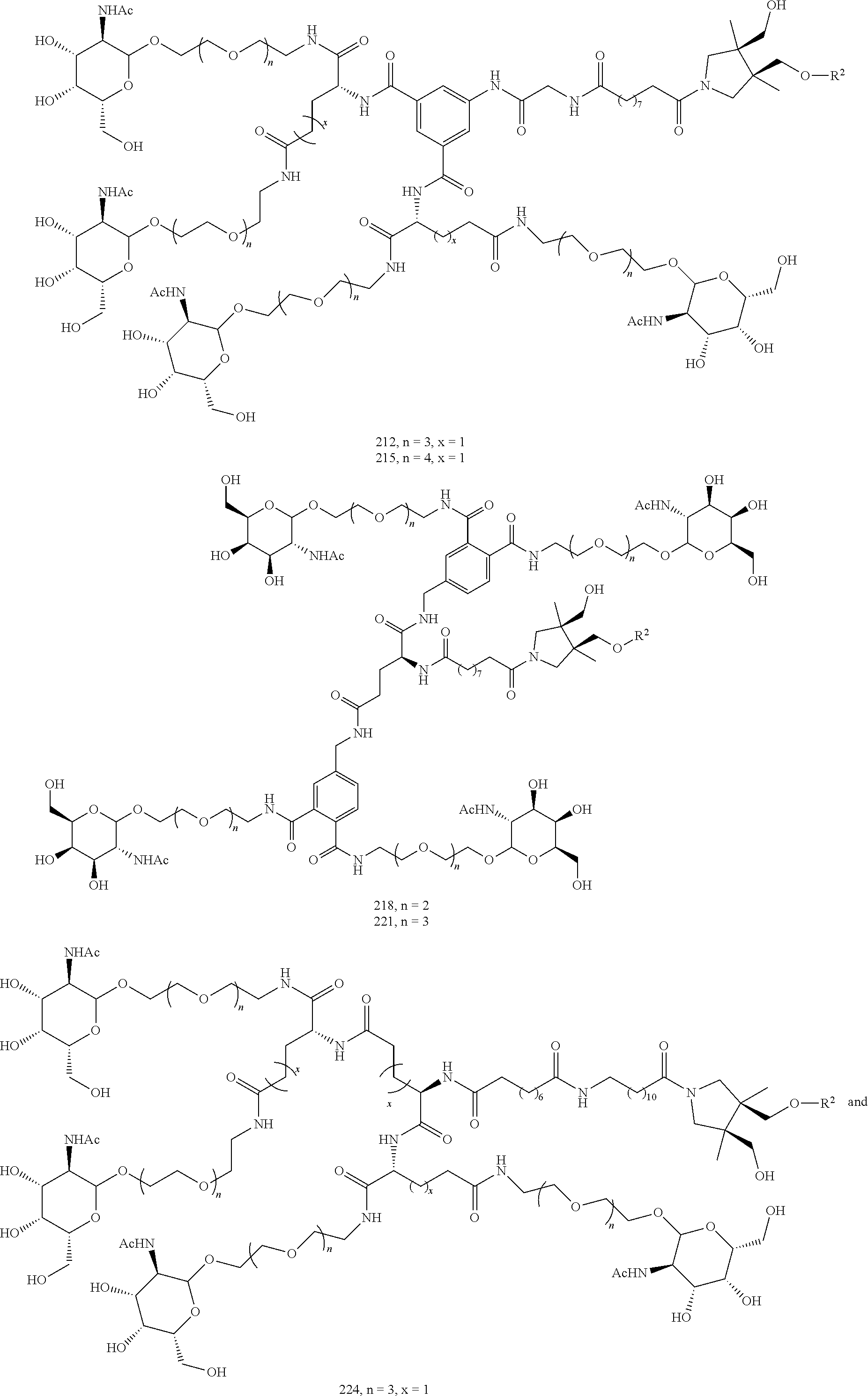

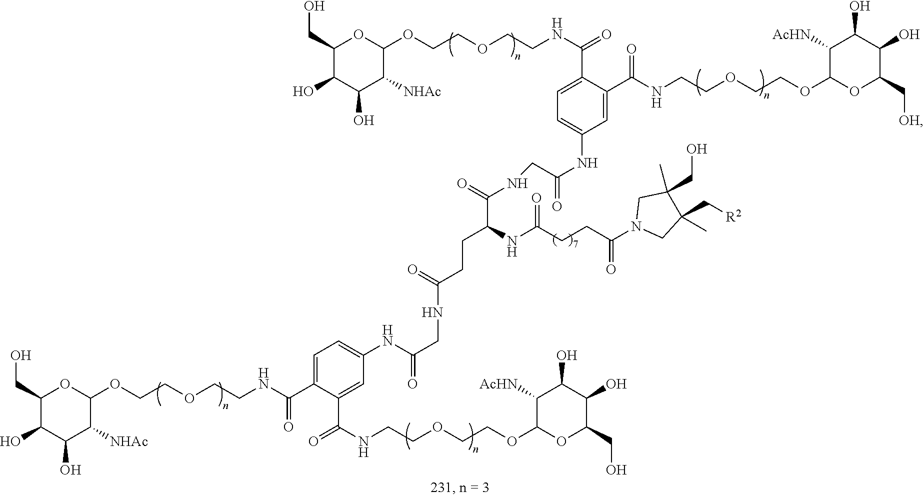

wherein R² is a double stranded siRNA molecule.

In certain embodiments, the conjugate is

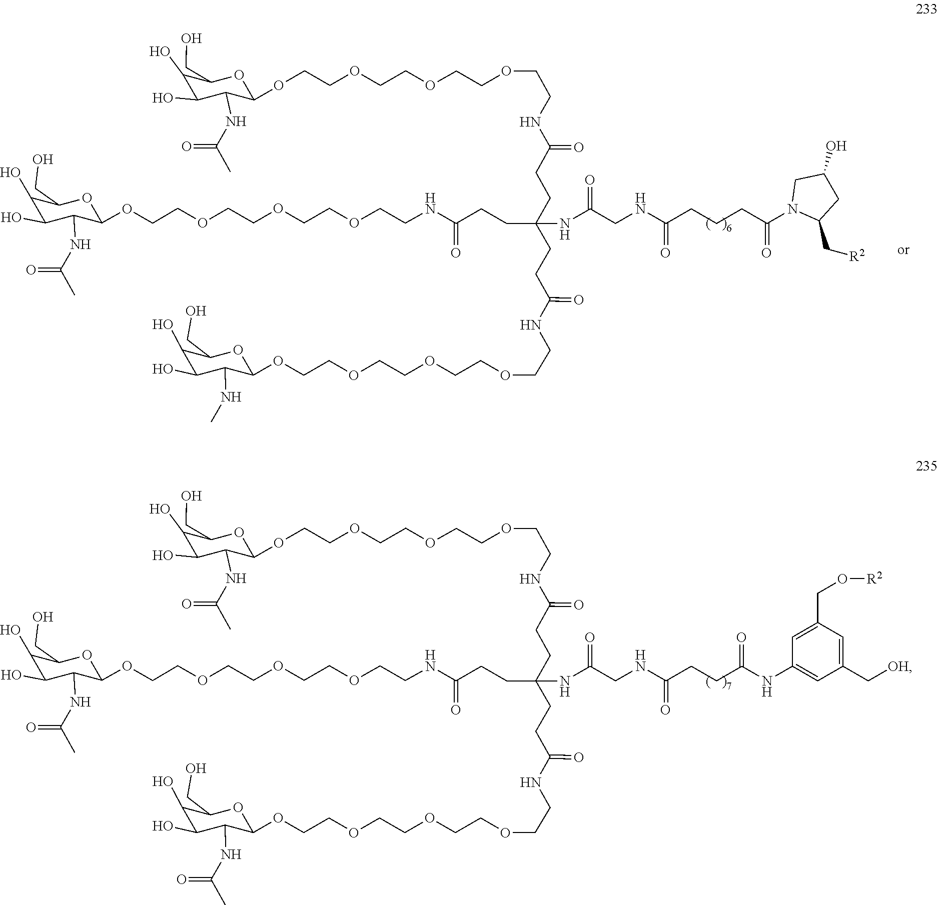

wherein R² is a double stranded siRNA molecule.

In certain embodiments, the conjugate is a GalNAc conjugate:

A-B-C

wherein A is a targeting ligand;
B is an optional linker; and
C is an siRNA molecule.

In certain embodiments, the conjugate is

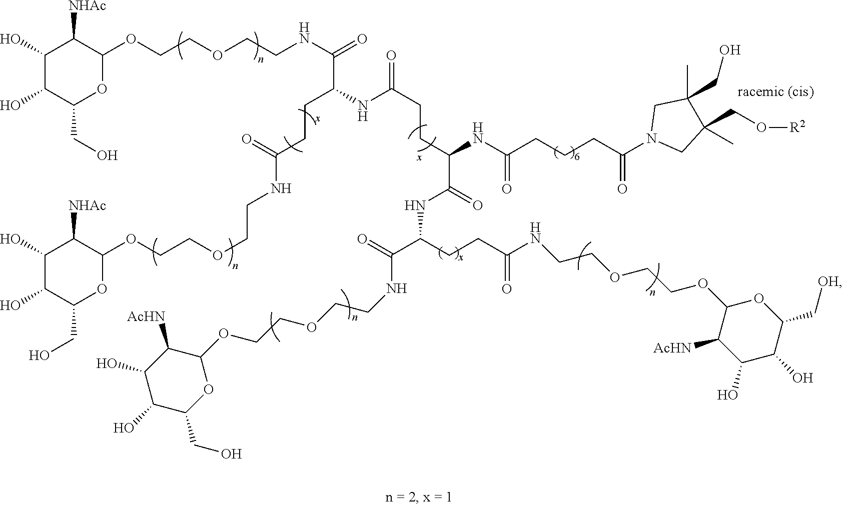

wherein R² is a double stranded siRNA molecule.

In certain embodiments, the conjugate is

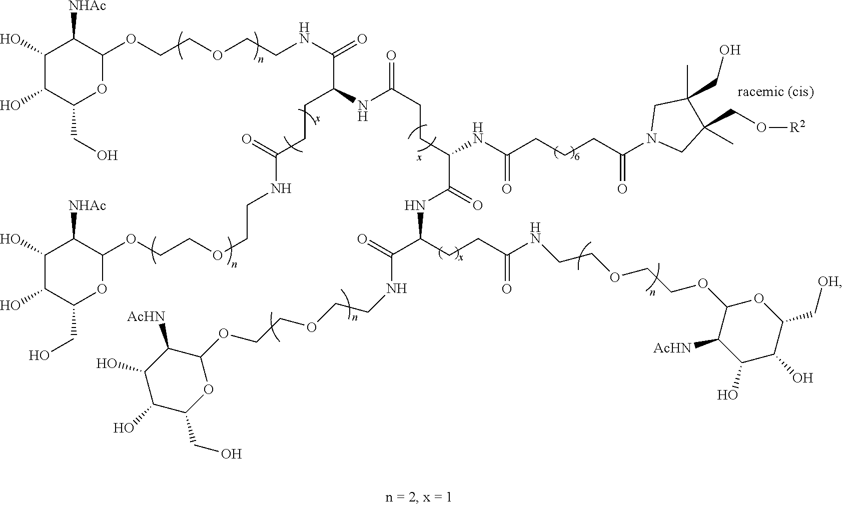

wherein R² is a double stranded siRNA molecule.

In certain embodiments, the conjugate is

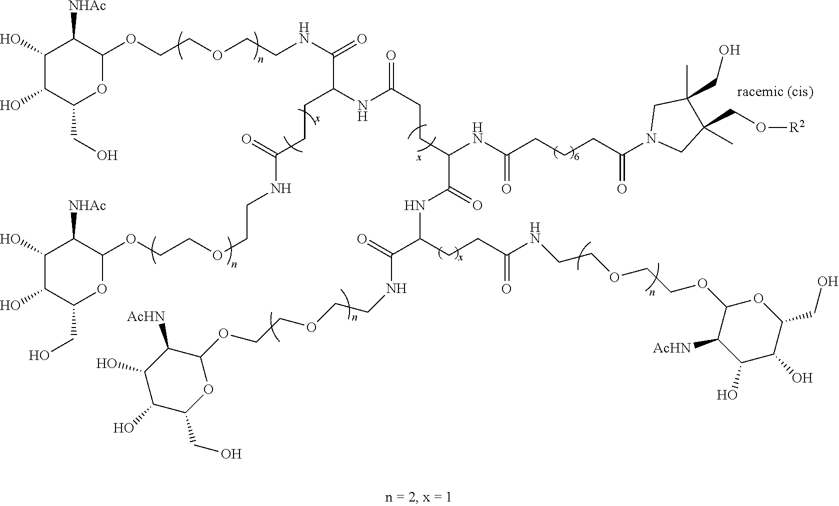

wherein R² is a double stranded siRNA molecule.

In certain embodiments, the conjugate is

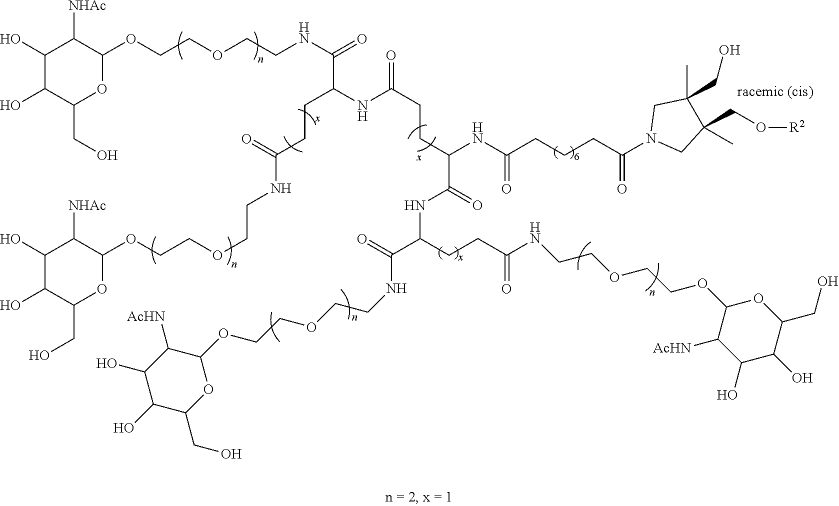

wherein R² is a double stranded siRNA molecule.

In certain embodiments, the conjugate is

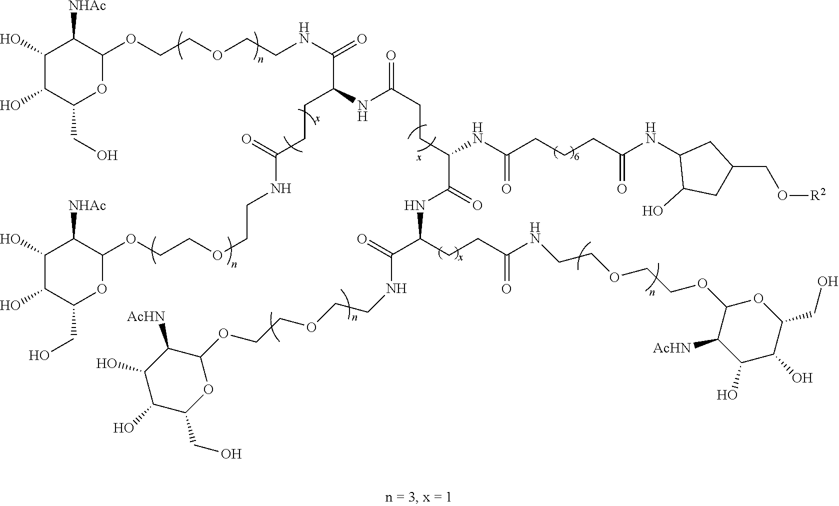

wherein R² is a double stranded siRNA molecule.

In certain embodiments, the conjugate is

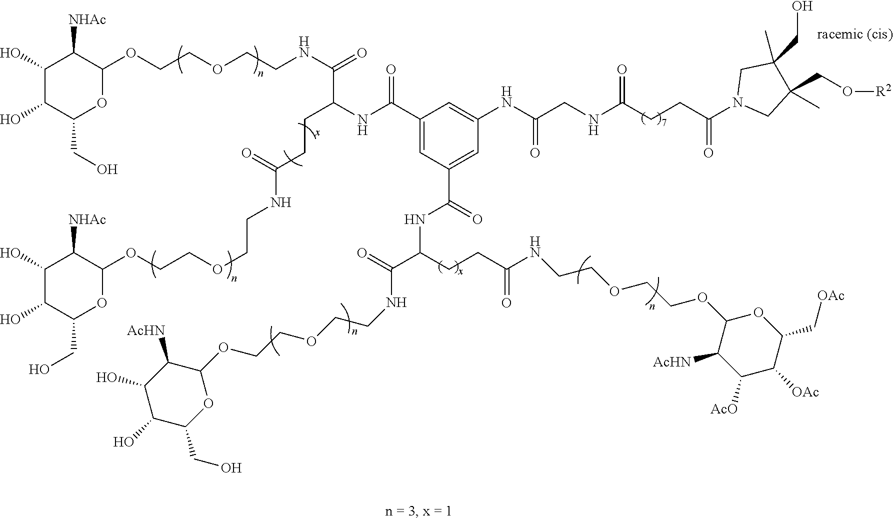

wherein R² is a double stranded siRNA molecule.

In certain embodiments, the conjugate is

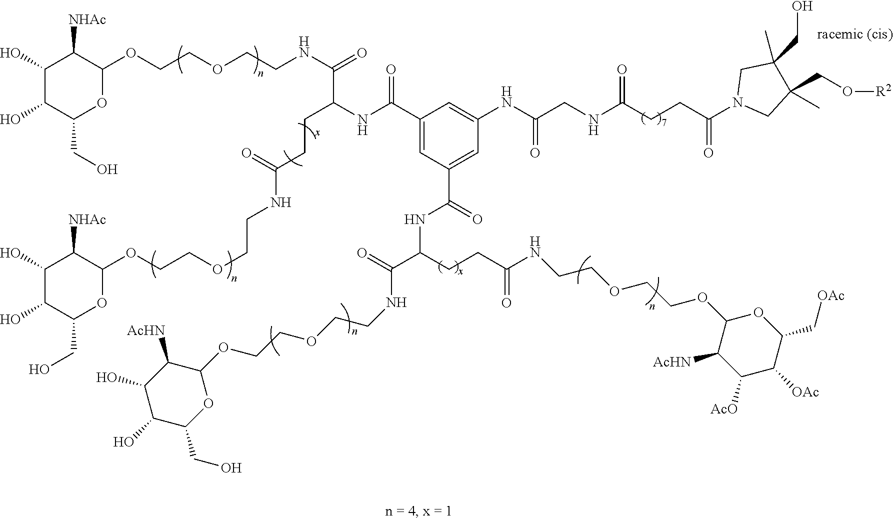

wherein R² is a double stranded siRNA molecule.

In certain embodiments, the conjugate is

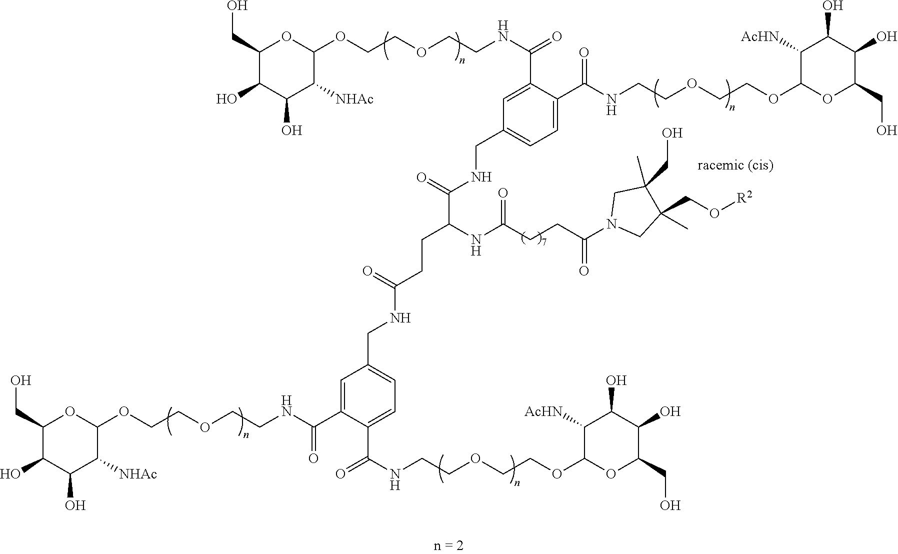

wherein R² is a double stranded siRNA molecule.

In certain embodiments, the conjugate is

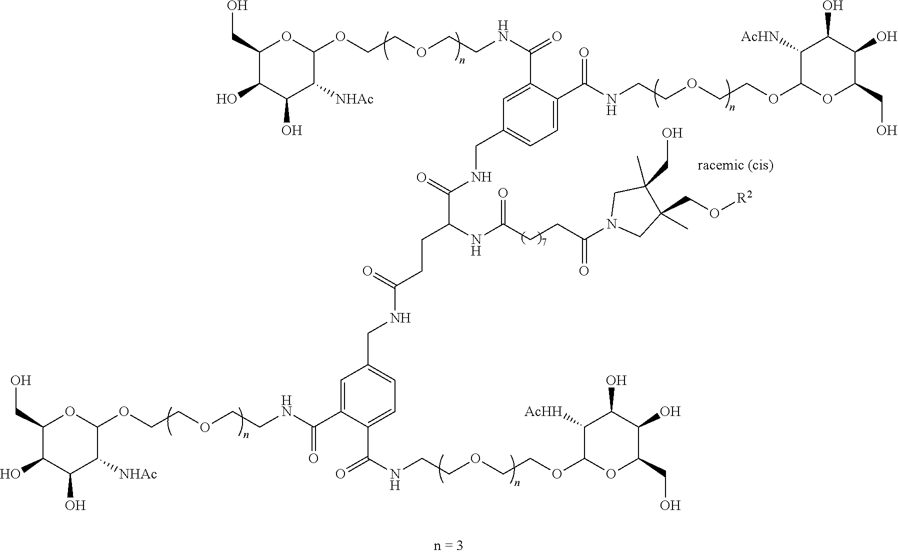

wherein R² is a double stranded siRNA molecule.

In certain embodiments, the conjugate is

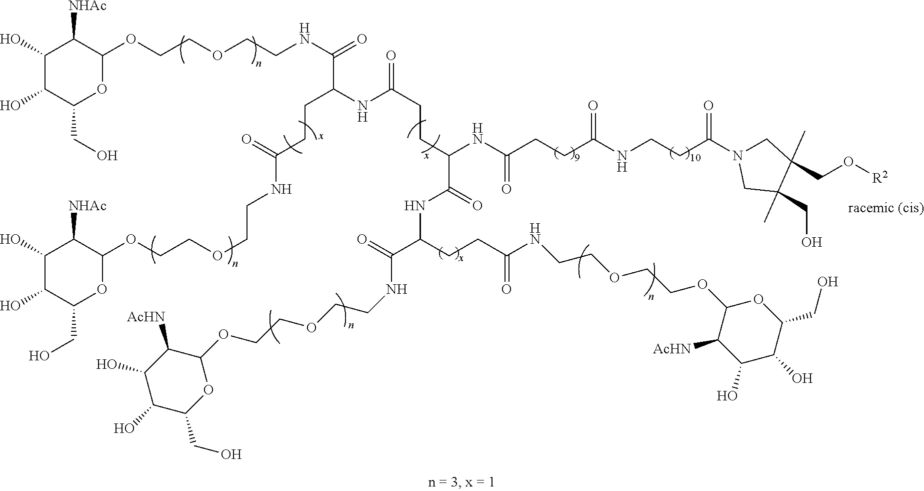

wherein R² is a double stranded siRNA molecule.

In certain embodiments, the conjugate is

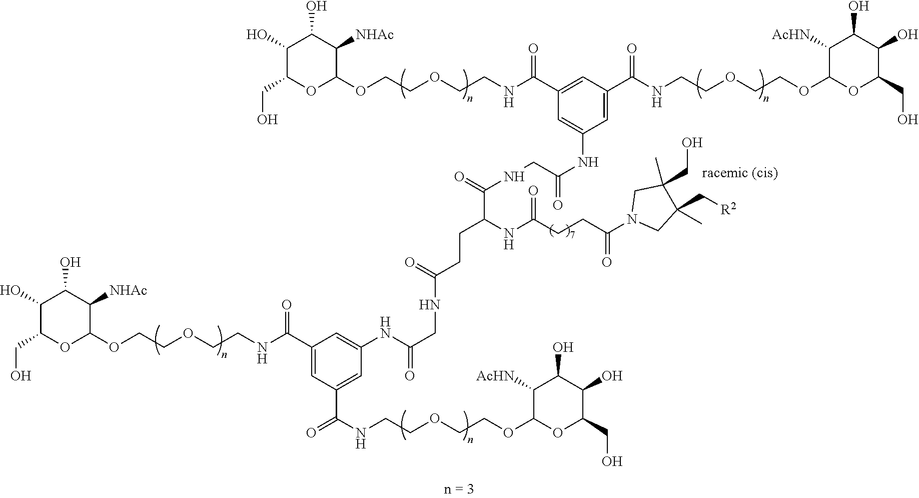

wherein R² is a double stranded siRNA molecule.

In certain embodiments, the conjugate is

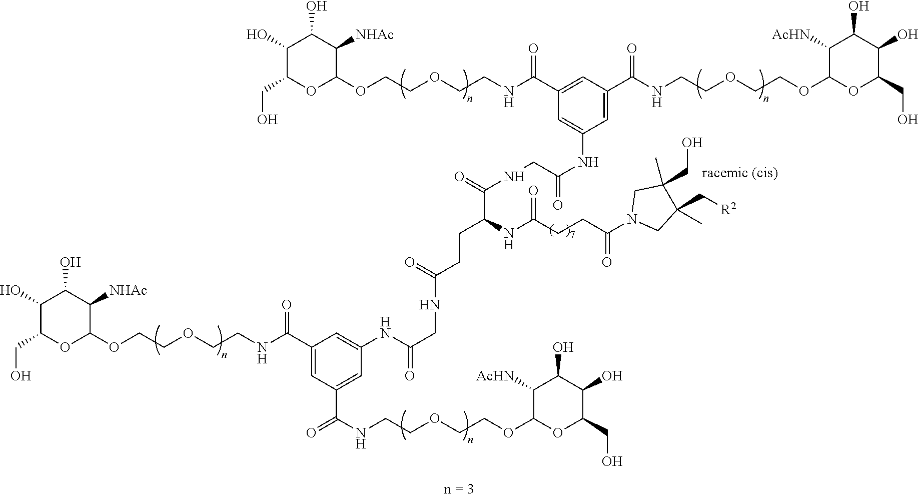

wherein R² is a double stranded siRNA molecule.

In certain embodiments, the conjugate is

wherein R² is a double stranded siRNA molecule.

In certain embodiments, the conjugate is

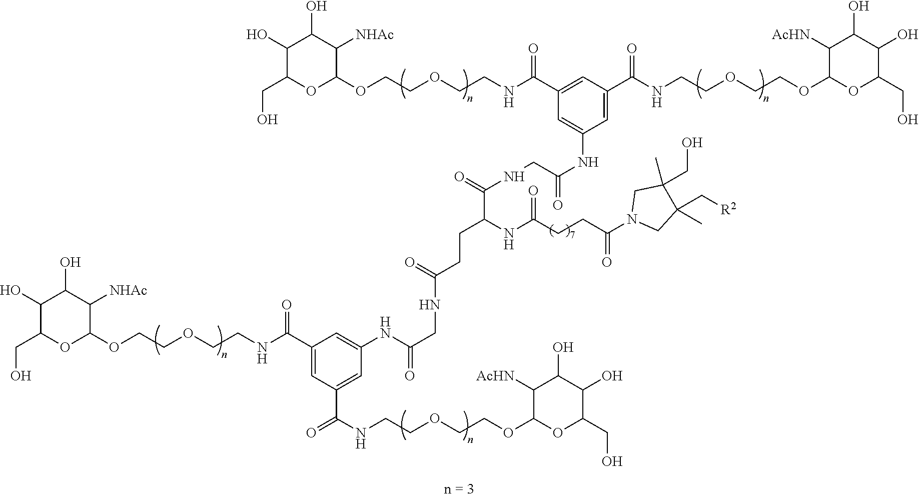

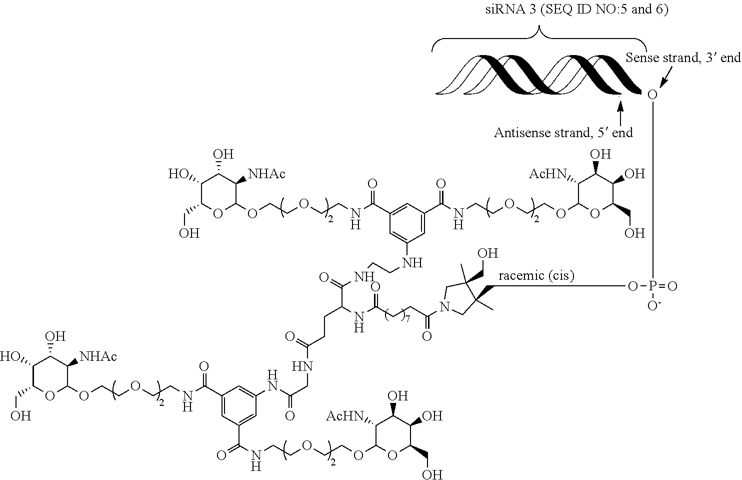

In certain embodiments, the conjugate is

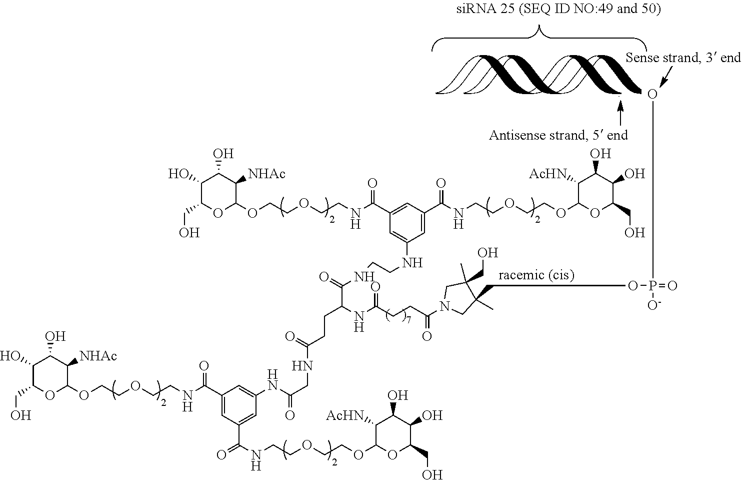

In certain embodiments, the conjugate is

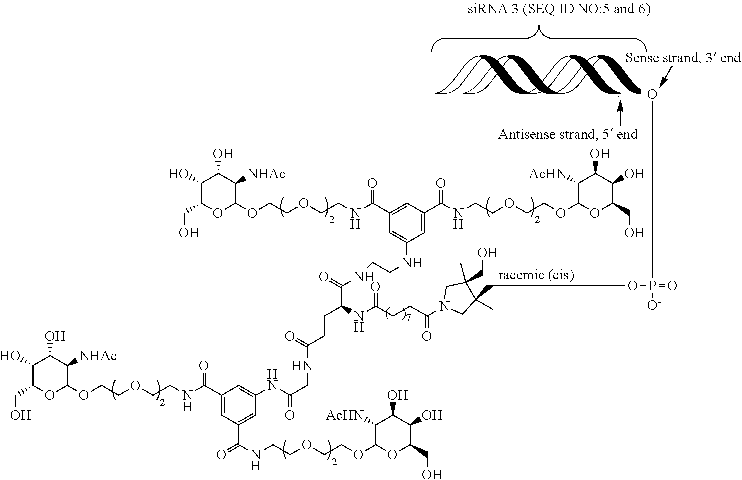

In certain embodiments, the conjugate is

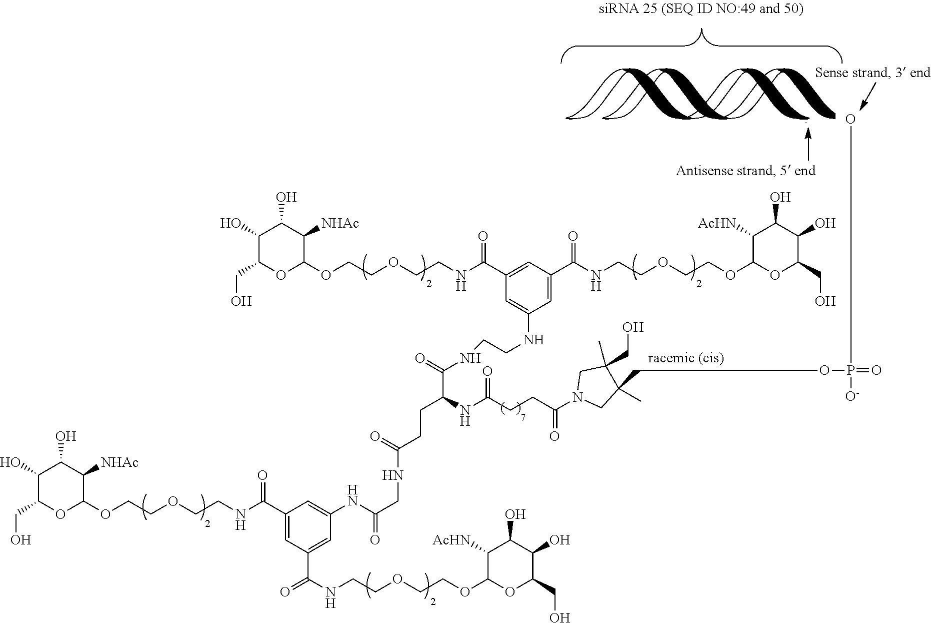

In certain embodiments, the conjugate is

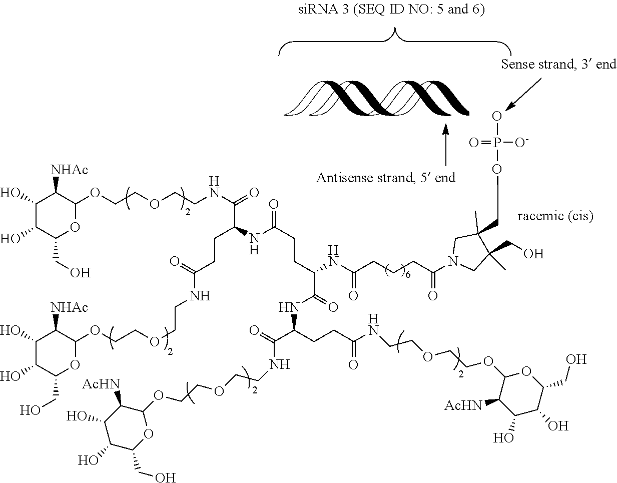

In certain embodiments, the conjugate is

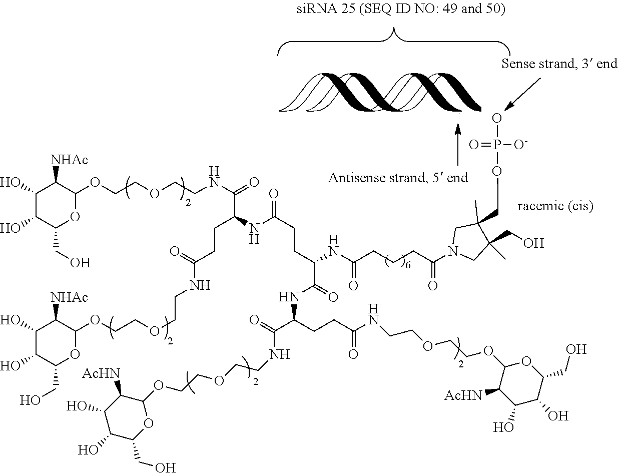

In certain embodiments, the conjugate is

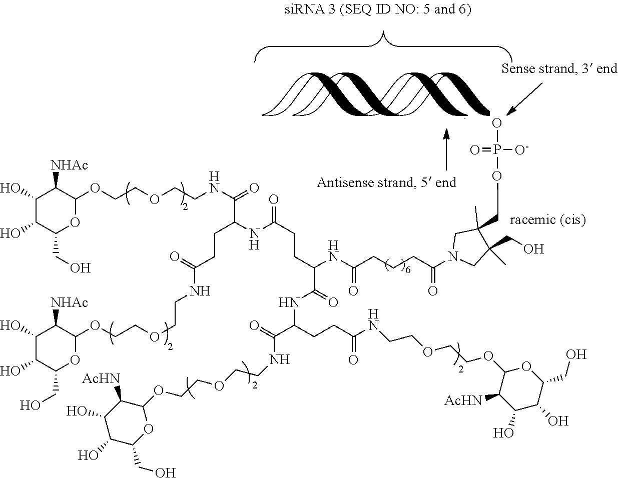

In certain embodiments, the conjugate is

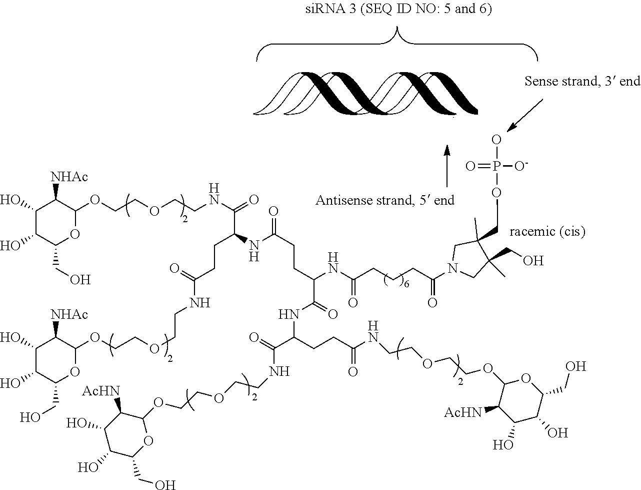

In certain embodiments, the conjugate is

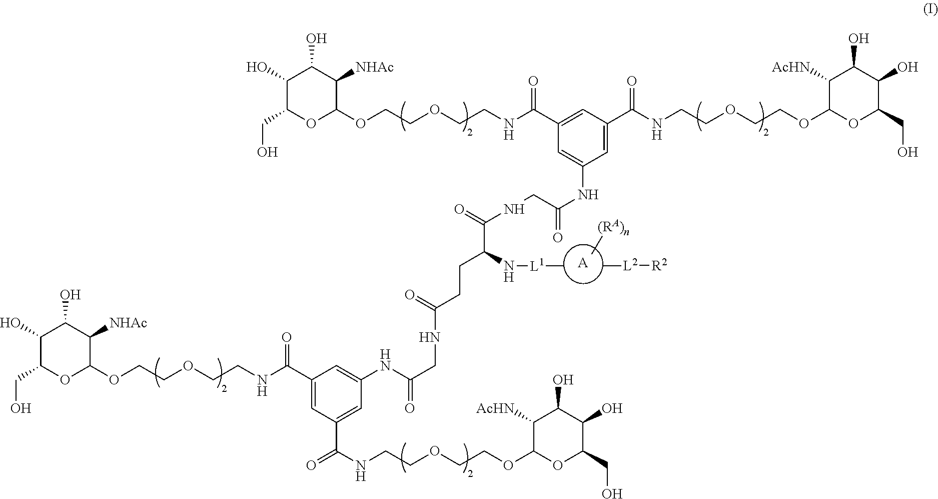

wherein the following definitions apply:

L¹ is absent or a linking group;

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen or a protecting group; and

n is 0, 1, 2, 3, 4, 5, 6, 7, 8, 9, or 10;

or a salt thereof.

In certain embodiments, the conjugate is

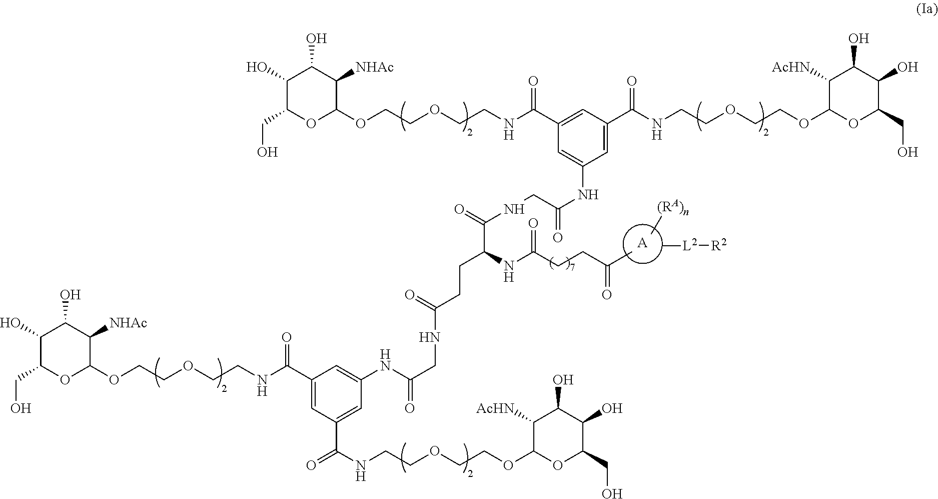

wherein the following definitions apply:

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl; each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen or a protecting group; and

n is 0, 1, 2, 3, 4, 5, 6, 7, 8, 9, or 10;

or a salt thereof.

In certain embodiments, the conjugate is

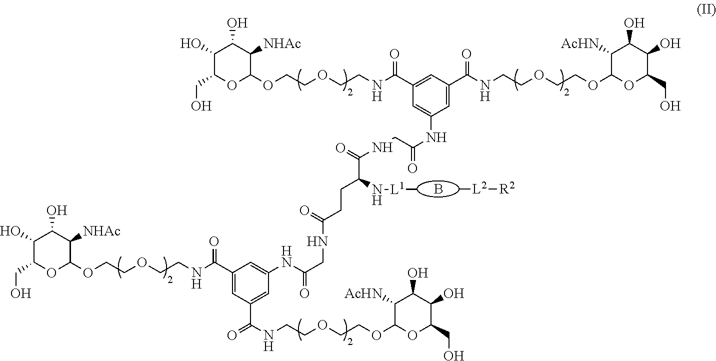

wherein the following definitions apply:

L¹ is absent or a linking group;

L² is absent or a linking group;

R² is a nucleic acid;

B is divalent and is selected from the group consisting of:

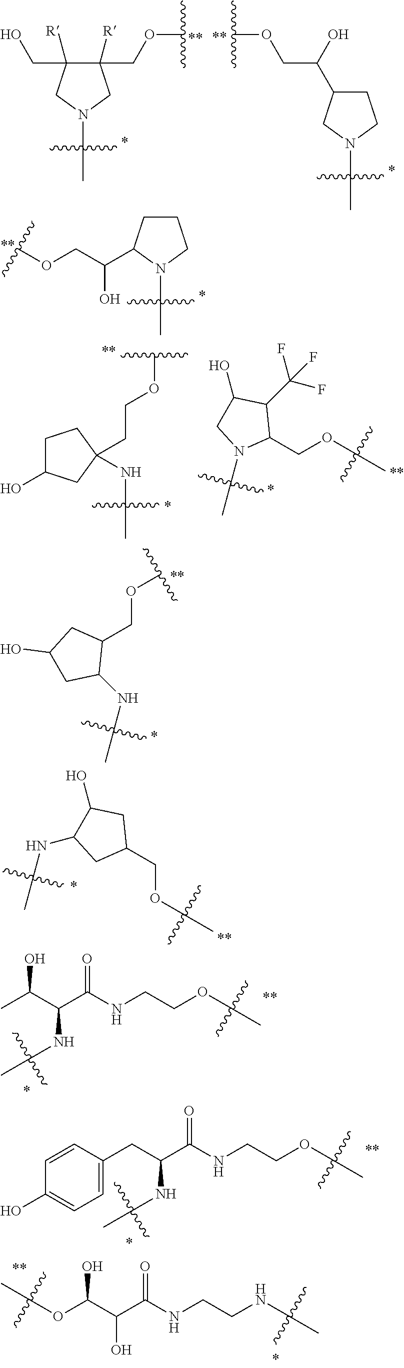

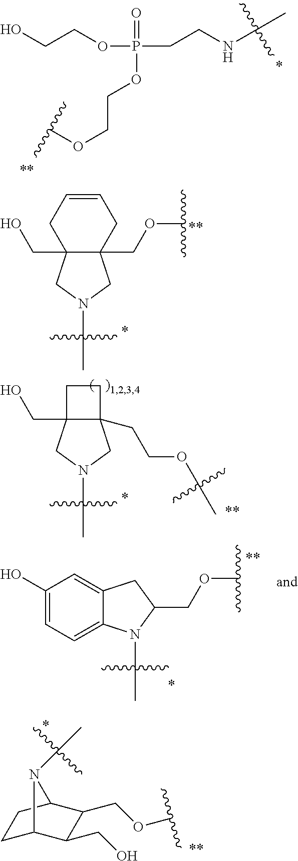

wherein:

each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;

the valence marked with * is attached to L¹ or is attached to R¹ if L¹ is absent; and

the valence marked with ** is attached to L² or is attached to R² if L² is absent;

or a salt thereof.

In certain embodiments, L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In certain embodiments, L¹ is selected from the group consisting of:

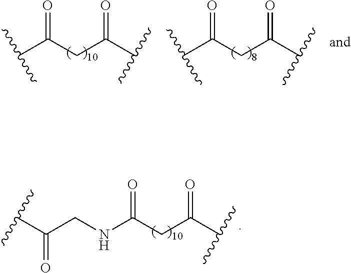

In certain embodiments, L¹ is connected to B¹ through a linkage selected from the group consisting of: —O—, —S—, —(C═O)—, —(C═O)—NH—, —NH—(C═O), —(C═O)—O—, —NH—(C═O)—NH—, or —NH—(SO₂)—.

In certain embodiments, L¹ is selected from the group consisting of:

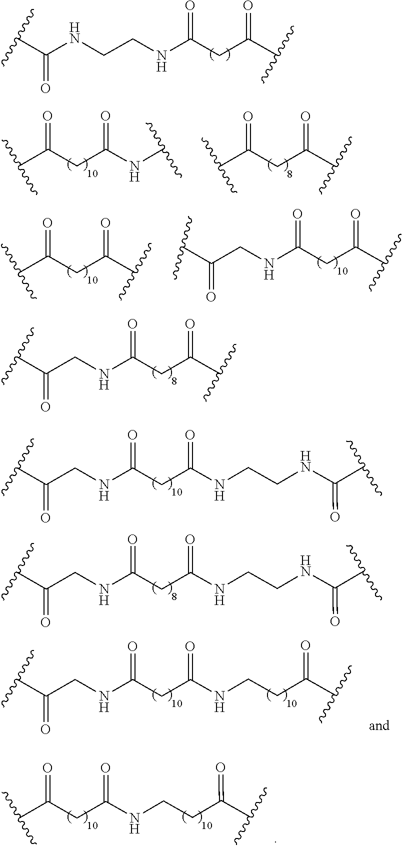

In certain embodiments, L² is connected to R² through —O—.

In certain embodiments, L² is C₁₋₄ alkylene-O— that is optionally substituted with hydroxy.

In certain embodiments, L² is absent.

In certain embodiments, the conjugate is

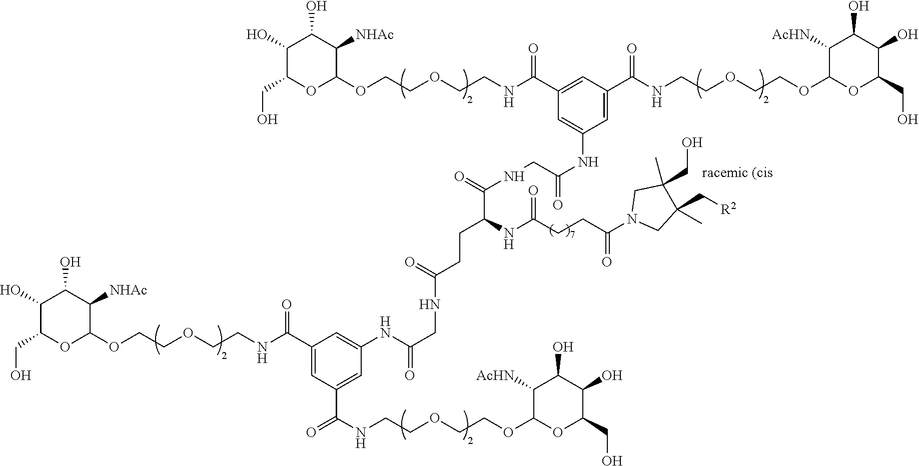

wherein R² is a nucleic acid.

In certain embodiments, the conjugate is

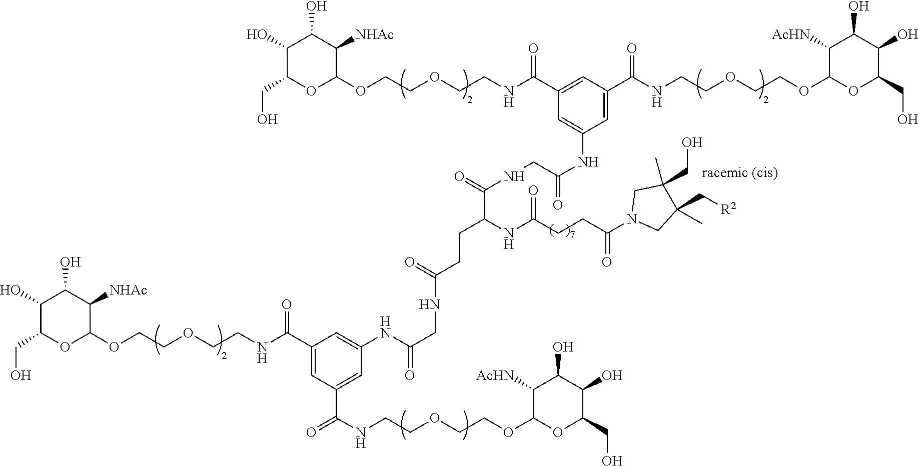

wherein R² is a nucleic acid.

In certain embodiments, the conjugate is

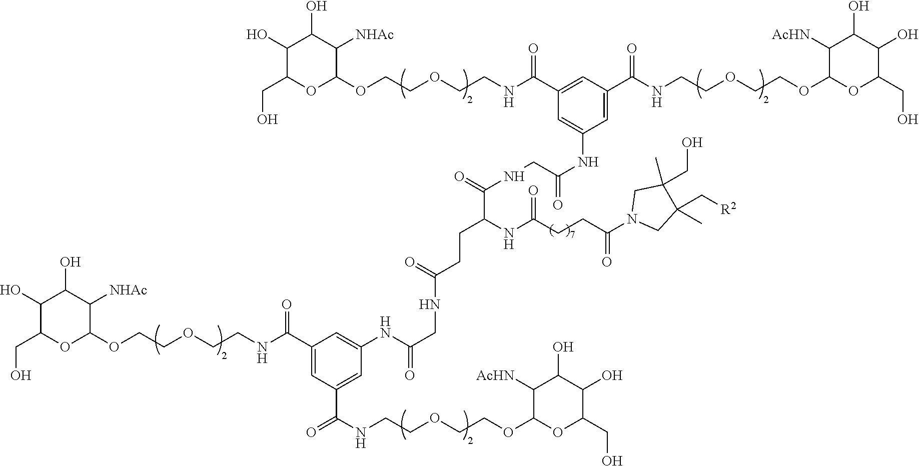

wherein R² is a nucleic acid.

In certain embodiments, the conjugate is a conjugate of the following formula:

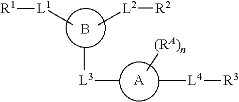

wherein the following definitions apply:

R¹ is a saccharide;

L¹ is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;

B is a 5-10 membered aryl or a 5-10 membered heteroaryl, which 5-10 membered aryl or 5-10 membered heteroaryl is optionally substituted with one or more groups independently selected from the group consisting of halo, hydroxy, cyano, trifluoromethyl, trifluoromethoxy, (C₁-C₆)alkyl, (C₁-C₆)alkoxy, (C₁-C₆)alkoxycarbonyl, (C₁-C₆)alkanoyloxy, (C₃-C₆)cycloalkyl, and (C₃-C₆)cycloalkyl(C₁-C₆)alkyl;

L² is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;

R² is a saccharide;

L³ is absent or a linking group;

A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{a}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

n is 0, 1, 2, 3, 4, 5, 6, 7, 8, 9, or 10;

L⁴ is absent or a linking group;

R³ is a nucleic acid;

R^{a} is hydrogen, a protecting group, a covalent bond to a solid support, or a bond to a linking group L⁵ that is bound to a solid support; and

L⁵ is a linking group;

or a salt thereof.

In certain embodiments, A is absent.

In certain embodiments, A is a 3-20 membered cycloalkyl, a 5-20 membered aryl, a 5-20 membered heteroaryl, or a 3-20 membered heterocycloalkyl.

In certain embodiments, B is a 5-10 membered aryl.

In certain embodiments, B is naphthyl or phenyl.

In certain embodiments, B is phenyl.

In certain embodiments, the group:

is:

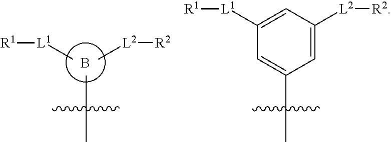

In certain embodiments, B is a 5-10 membered heteroaryl.

In certain embodiments, B is pyridyl, pyrimidyl, quinolyl, isoquinolyl, imidazoyl, thiazolyl, dioxazoyl or oxazolyl.

In certain embodiments, the group:

is:

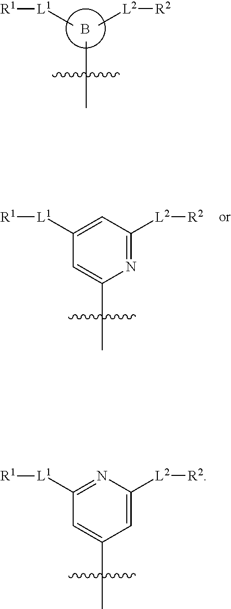

In certain embodiments, the group:

is:

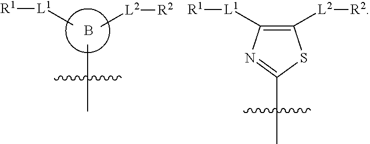

In certain embodiments, L¹ is a divalent, unbranched, saturated hydrocarbon chain, having from 0 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo.

In certain embodiments, L¹ is a divalent, unbranched, saturated hydrocarbon chain, having from 0 to 12 carbon atoms, wherein one or more (e.g. 1, 2, 3, or 4) of the carbon atoms in the hydrocarbon chain is optionally replaced by —O—, —NR^{X}—C(═O)—, or —C(═O)—NR^{X}—, and wherein R^{X} is hydrogen or (C₁-C₆)alkyl.

In certain embodiments, L¹ is:
—C(═O)N(H)—CH₂CH₂OCH₂CH₂OCH₂CH₂—,
—C(═O)N(H)—CH₂CH₂OCH₂CH₂OCH₂CH₂OCH₂CH₂—,
—C(═O)N(CH₃)—CH₂CH₂OCH₂CH₂OCH₂CH₂—, or
—C(═O)N(CH₃)—CH₂CH₂OCH₂CH₂OCH₂CH₂OCH₂CH₂—.

In certain embodiments, L² is a divalent, unbranched, saturated hydrocarbon chain, having from 0 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo.

In certain embodiments, L² is a divalent, unbranched, saturated hydrocarbon chain, having from 0 to 12 carbon atoms, wherein one or more (e.g. 1, 2, 3, or 4) of the carbon atoms in the hydrocarbon chain is optionally replaced by —O—, —NR^{X}—C(═O)—, or —C(═O)—NR^{X}—, and wherein R^{X} is hydrogen or (C₁-C₆)alkyl.

In certain embodiments, L² is:
—C(═O)N(H)—CH₂CH₂OCH₂CH₂OCH₂CH₂—,
—C(═O)N(H)—CH₂CH₂OCH₂CH₂OCH₂CH₂OCH₂CH₂—,
—C(═O)N(CH₃)—CH₂CH₂OCH₂CH₂OCH₂CH₂—, or
—C(═O)N(CH₃)—CH₂CH₂OCH₂CH₂OCH₂CH₂OCH₂CH₂—.

In certain embodiments, R¹ is:

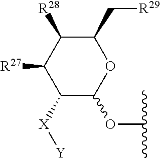

wherein:
X is NR²⁰ and Y is selected from —(C═O)R²¹, —SO₂R²², and —(C═O)NR²³R²⁴; or X is —(C═O)— and Y is NR²⁵R²⁶; or X is —NR³⁷R³⁸ and Y is absent
R²⁰ is hydrogen or (C₁-C₄)alkyl;
R²¹, R²², R²³, R²⁴, R²⁵ and R²⁶ are each independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo, (C₁-C₄)alkyl, and (C₁-C₄)alkoxy;
R²⁷ is —OH, —NR²⁵R²⁶ or —F;
R²⁸ is —OH, —NR²⁵R²⁶ or —F;
R²⁹ is —OH, —NR²⁵R²⁶, —F, —N₃, —NR³⁵R³⁶, or 5 membered heterocycle that is optionally substituted with one or more groups independently selected from the group consisting of halo, hydroxyl, carboxyl, amino, (C₁-C₄)alkyl, aryl, and (C₁-C₄)alkoxy, wherein any (C₁-C₄)alkyl, and (C₁-C₄)alkoxy is optionally substituted with one or more groups independently selected from the group consisting of halo, and wherein any aryl is optionally substituted with one or more groups independently selected from the group consisting of halo, hydroxyl, nitro, cyano, amino, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy;
each R³⁵ and R³⁶ is independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo and (C₁-C₄)alkoxy; or R³⁵ and R³⁶ taken together with the nitrogen to which they are attached form a 5-6 membered heteroaryl ring, which heteroaryl ring is optionally substituted with one or more groups independently selected from the group consisting of (C₁-C₈)alkyl, (C₁-C₈)alkoxy, aryl, and (C₃-C₆)cycloalkyl, wherein any aryl, and (C₃-C₆)cycloalkyl is optionally substituted with one or more groups R³⁹;
each R³⁷ and R³¹ is independently selected from the group consisting of hydrogen, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy; or R³⁷ and R³⁸ taken together with the nitrogen to which they are attached form a 5-8 membered heterocycle that is optionally substituted with one or more groups independently selected from the group consisting of halo, hydroxyl, carboxyl, amino, oxo (═O), (C₁-C₄)alkyl, and (C₁-C₄)alkoxy, wherein any (C₁-C₄)alkyl, and (C₁-C₄)alkoxy is optionally substituted with one or more groups independently selected from halo; and
each R³⁹ is independently selected from the group consisting of (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from halo.

In certain embodiments, R¹ is:

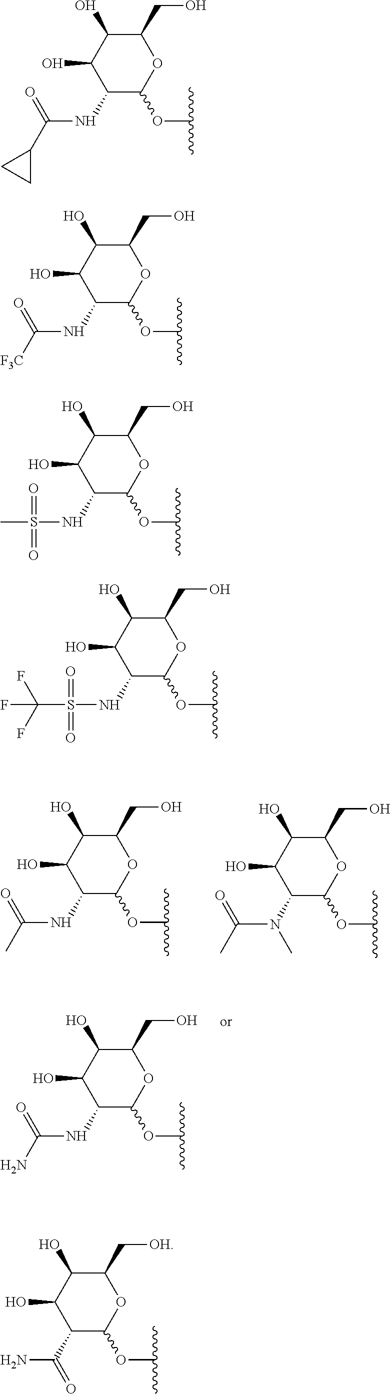

In certain embodiments, R¹ is:

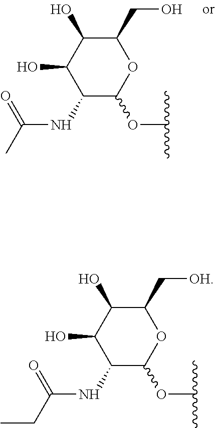 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In certain embodiments, R¹ is:

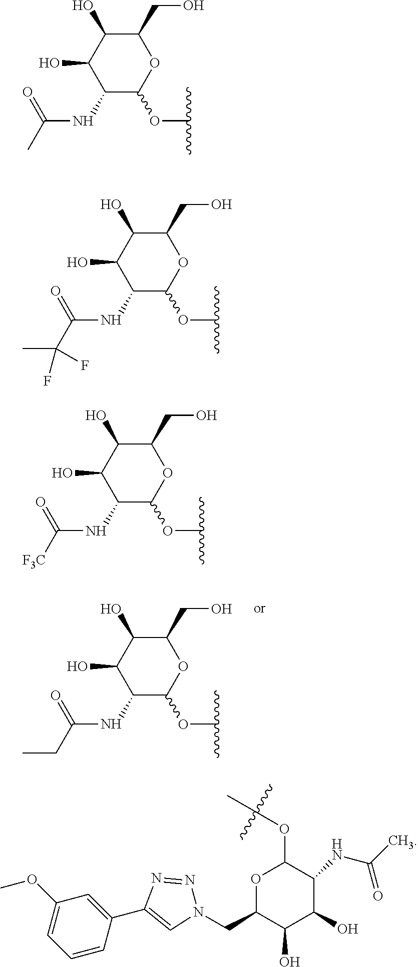 [SMILES: *OC1O[C@H](CO)[C@H](O)[C@H](O)[C@H]1NC(C)=O ; *OC1O[C@H](CO)[C@H](O)[C@H](O)[C@H]1NC(=O)C(C)(F)F ; *OC1O[C@H](CO)[C@H](O)[C@H](O)[C@H]1NC(=O)C(F)(F)F ; *OC1O[C@H](CO)[C@H](O)[C@H](O)[C@H]1NC(=O)CC ; *OC1O[C@H](Cn2cc(-c3cccc(OC)c3)nn2)[C@H](O)[C@H](O)[C@H]1NC(C)=O]

In certain embodiments, R¹ is:

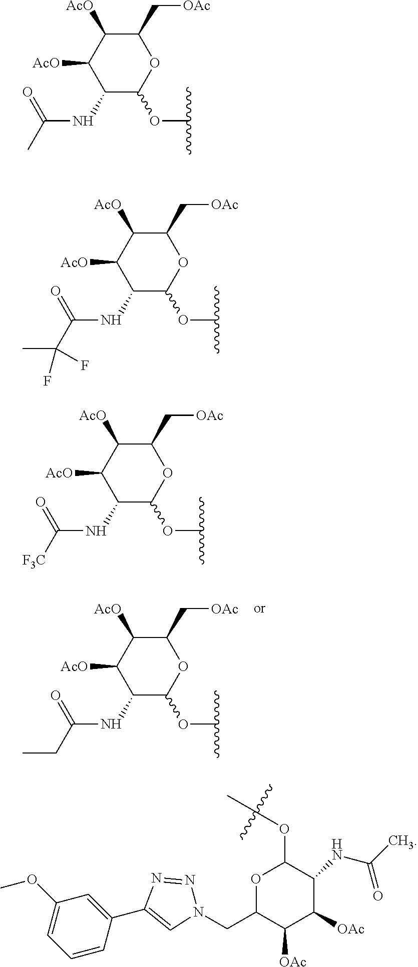 [SMILES: *OC1O[C@H](COC(C)=O)[C@H](OC(C)=O)[C@H](OC(C)=O)[C@H]1NC(C)=O ; *OC1O[C@H](COC(C)=O)[C@H](OC(C)=O)[C@H](OC(C)=O)[C@H]1NC(=O)C(C)(F)F ; *OC1O[C@H](COC(C)=O)[C@H](OC(C)=O)[C@H](OC(C)=O)[C@H]1NC(=O)C(F)(F)F ; *OC1O[C@H](COC(C)=O)[C@H](OC(C)=O)[C@H](OC(C)=O)[C@H]1NC(=O)CC ; *OC1OC(Cn2cc(-c3cccc(OC)c3)nn2)[C@H](OC(C)=O)[C@H](OC(C)=O)[C@H]1NC(C)=O]

In certain embodiments, R² is:

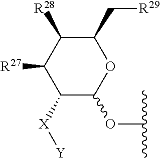

wherein:
X is NR²⁰ and Y is selected from —(C═O)R²¹, —SO₂R²², and —(C═O)NR²³R²⁴; or X is —(C═O)— and Y is NR²⁵R²⁶; or X is —NR³⁷R³⁸ and Y is absent
R²⁰ is hydrogen or (C₁-C₄)alkyl;
R²¹, R²², R²³, R²⁴, R²⁵ and R²⁶ are each independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo, (C₁-C₄)alkyl, and (C₁-C₄)alkoxy;
R²⁷ is —OH, —NR²⁵R²⁶ or —F;
R²⁸ is —OH, —NR²⁵R²⁶ or —F;
R²⁹ is —OH, —NR²⁵R²⁶, —F, —N₃, —NR³⁵R³⁶, or 5 membered heterocycle that is optionally substituted with one or more groups independently selected from the group consisting of halo, hydroxyl, carboxyl, amino, (C₁-C₄)alkyl, aryl, and (C₁-C₄)alkoxy, wherein any (C₁-C₄)alkyl, and (C₁-C₄)alkoxy is optionally substituted with one or more groups independently selected from the group consisting of halo, and wherein any aryl is optionally substituted with one or more groups independently selected from the group consisting of halo, hydroxyl, nitro, cyano, amino, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy;
each R³⁵ and R³⁶ is independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo and (C₁-C₄)alkoxy; or R³⁵ and R³⁶ taken together with the nitrogen to which they are attached form a 5-6 membered heteroaryl ring, which heteroaryl ring is optionally substituted with one or more groups independently selected from the group consisting of (C₁-C₈)alkyl, (C₁-C₈)alkoxy, aryl, and (C₃-C₆)cycloalkyl, wherein any aryl, and (C₃-C₆)cycloalkyl is optionally substituted with one or more groups R³⁹
each R³⁷ and R³⁸ is independently selected from the group consisting of hydrogen, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy; or R³⁷ and R³⁸ taken together with the nitrogen to which they are attached form a 5-8 membered heterocycle that is optionally substituted with one or more groups independently selected from the group consisting of halo, hydroxyl, carboxyl, amino, oxo (═O), (C₁-C₄)alkyl, and (C₁-C₄)alkoxy, wherein any (C₁-C₄)alkyl, and (C₁-C₄)alkoxy is optionally substituted with one or more groups independently selected from halo; and
each R³⁹ is independently selected from the group consisting of (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from halo.

In certain embodiments, R² is:

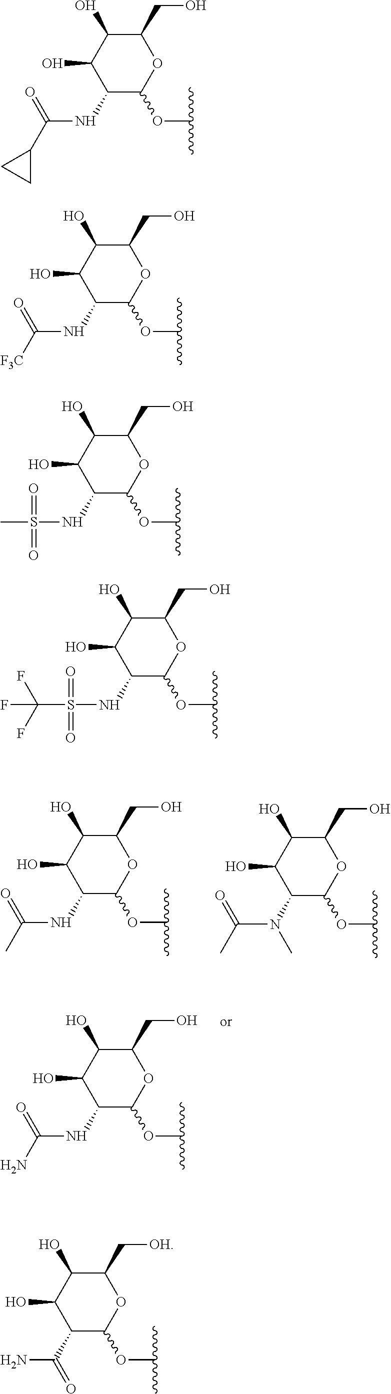

In certain embodiments, R² is:

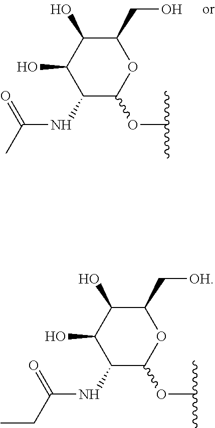 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In certain embodiments, R² is:

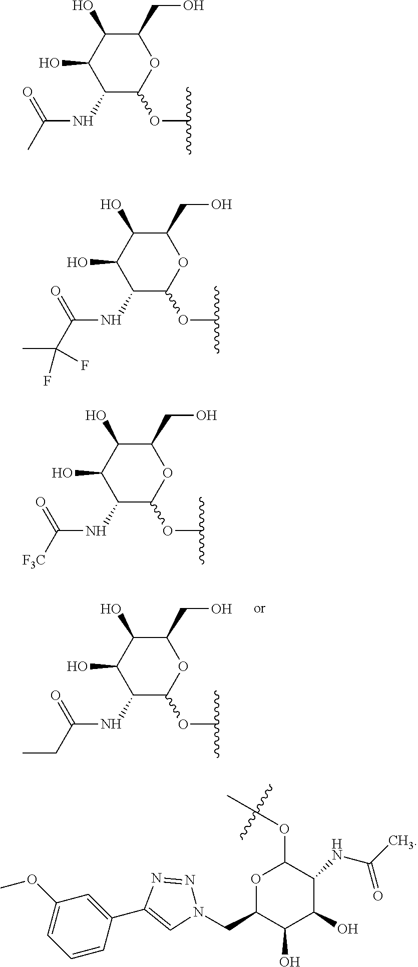 [SMILES: *OC1O[C@H](CO)[C@H](O)[C@H](O)[C@H]1NC(C)=O ; *OC1O[C@H](CO)[C@H](O)[C@H](O)[C@H]1NC(=O)C(C)(F)F ; *OC1O[C@H](CO)[C@H](O)[C@H](O)[C@H]1NC(=O)C(F)(F)F ; *OC1O[C@H](CO)[C@H](O)[C@H](O)[C@H]1NC(=O)CC ; *OC1O[C@H](Cn2cc(-c3cccc(OC)c3)nn2)[C@H](O)[C@H](O)[C@H]1NC(C)=O]

In certain embodiments, R² is:

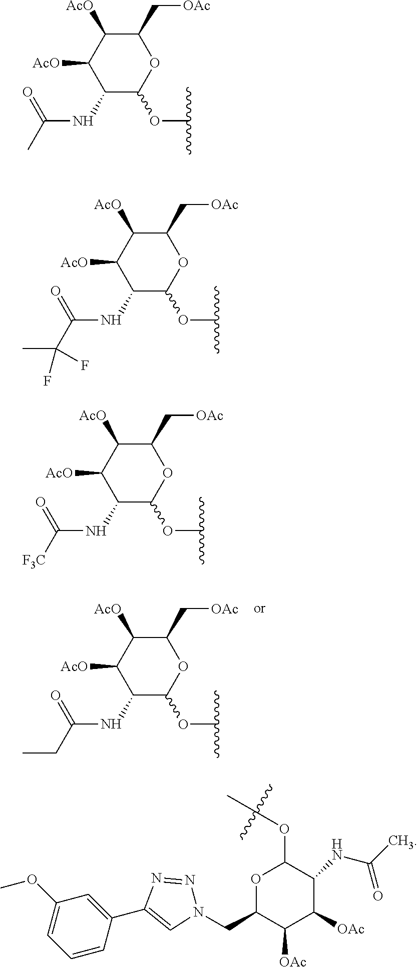 [SMILES: *OC1O[C@H](COC(C)=O)[C@H](OC(C)=O)[C@H](OC(C)=O)[C@H]1NC(C)=O ; *OC1O[C@H](COC(C)=O)[C@H](OC(C)=O)[C@H](OC(C)=O)[C@H]1NC(=O)C(C)(F)F ; *OC1O[C@H](COC(C)=O)[C@H](OC(C)=O)[C@H](OC(C)=O)[C@H]1NC(=O)C(F)(F)F ; *OC1O[C@H](COC(C)=O)[C@H](OC(C)=O)[C@H](OC(C)=O)[C@H]1NC(=O)CC ; *OC1O[C@H](Cn2cc(-c3cccc(OC)c3)nn2)[C@H](OC(C)=O)[C@H](OC(C)=O)[C@H]1NC(C)=O]

In certain embodiments, L³ is a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, L³ is a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, L³ is a divalent, branched or unbranched, saturated or unsaturated, hydrocarbon chain, having from 1 to 300 carbon atoms, wherein one or more of the carbon atoms is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more halo or oxo (═O).

In certain embodiments, L³ is:

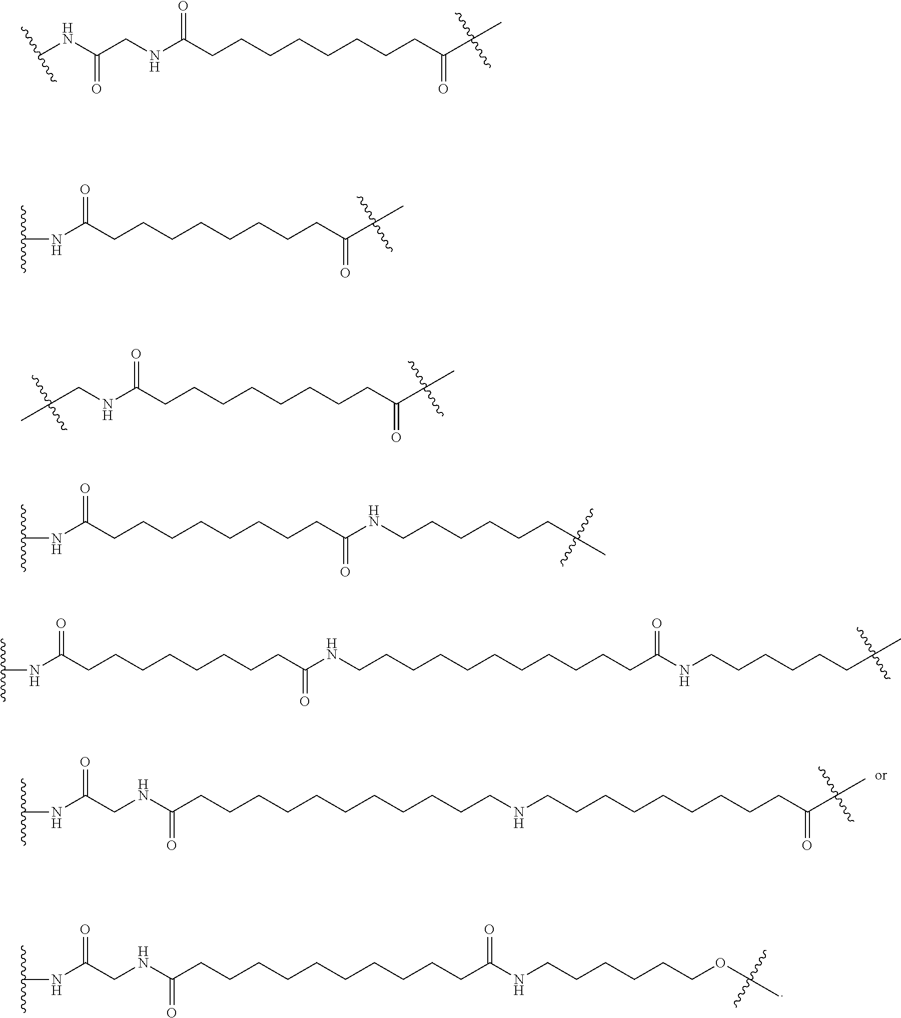

In certain embodiments, L³ is connected to B through —NH—, —O—, —S—, —(C═O)—, —(C═O)—NH—, —NH—(C═O)—, —(C═O)—O—, —NH—(C═O)—NH—, or —NH—(SO₂)—.

In certain embodiments, L⁴ is a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, L⁴ is a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In certain embodiments, L⁴ is a divalent, branched or unbranched, saturated or unsaturated, hydrocarbon chain, having from 1 to 300 carbon atoms, wherein one or more of the carbon atoms is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more halo or oxo (═O).

In certain embodiments, L⁴ is connected to R² through —O—.

In certain embodiments, the group:

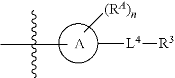

is selected from the group consisting of:

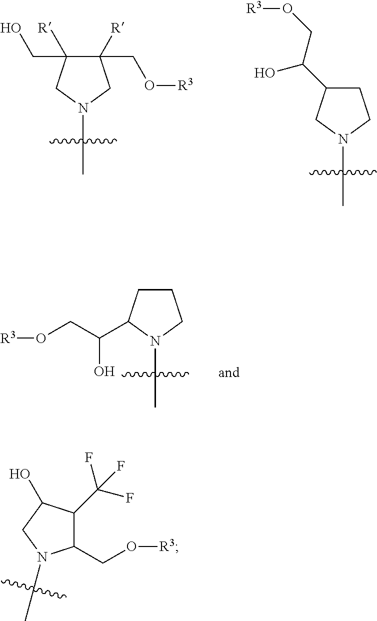

wherein
each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

In certain embodiments, the group:

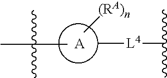

is selected from the group consisting of:

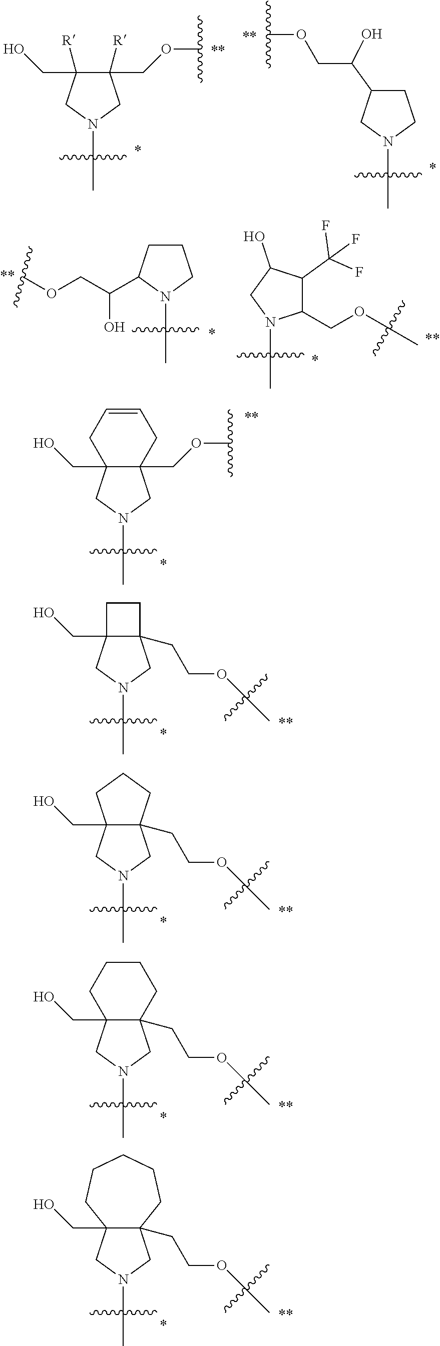

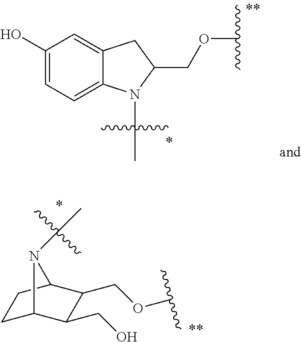

wherein:
each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;
the valence marked with * is attached to L³; and
the valence marked with ** is attached to R³.

In certain embodiments, the group:

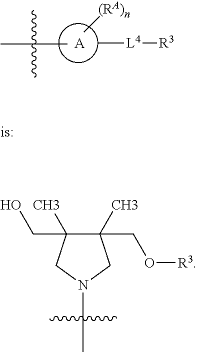

In certain embodiments, the conjugate is selected from the group consisting of:

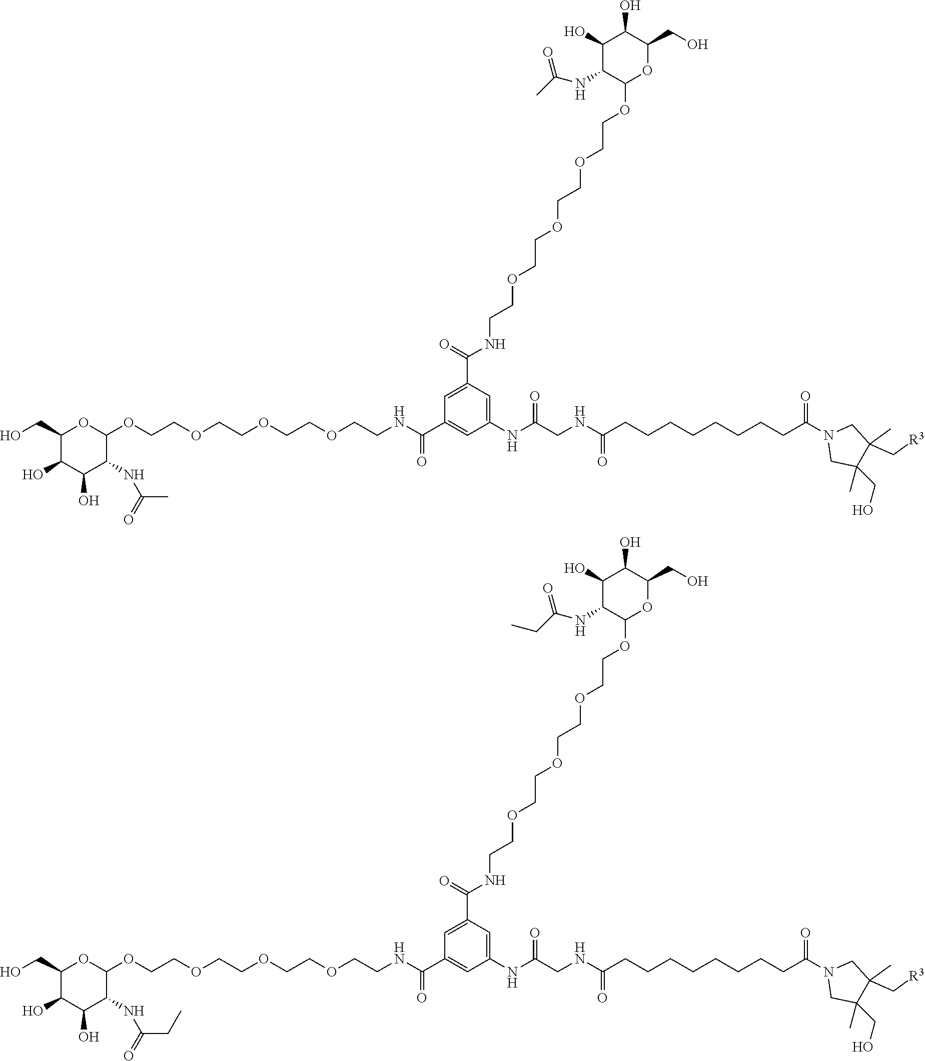

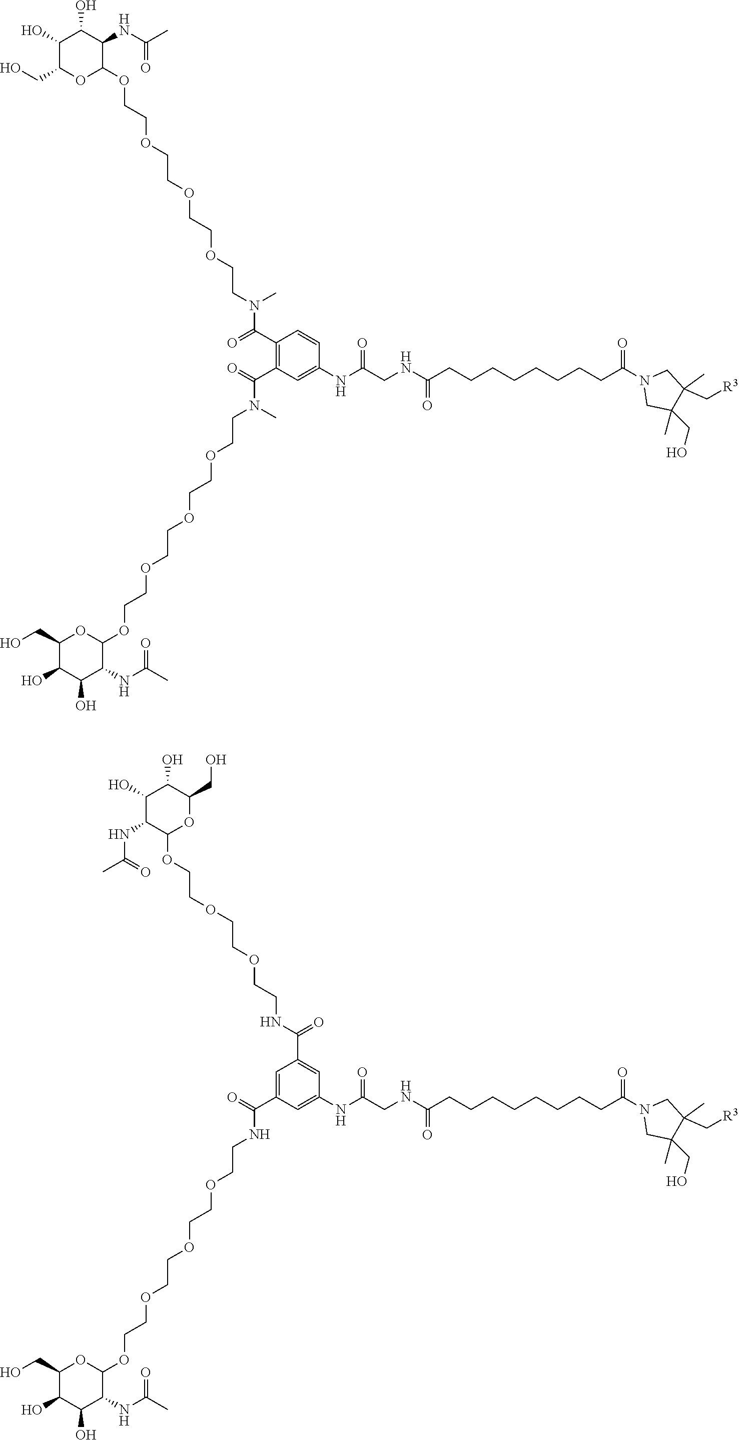

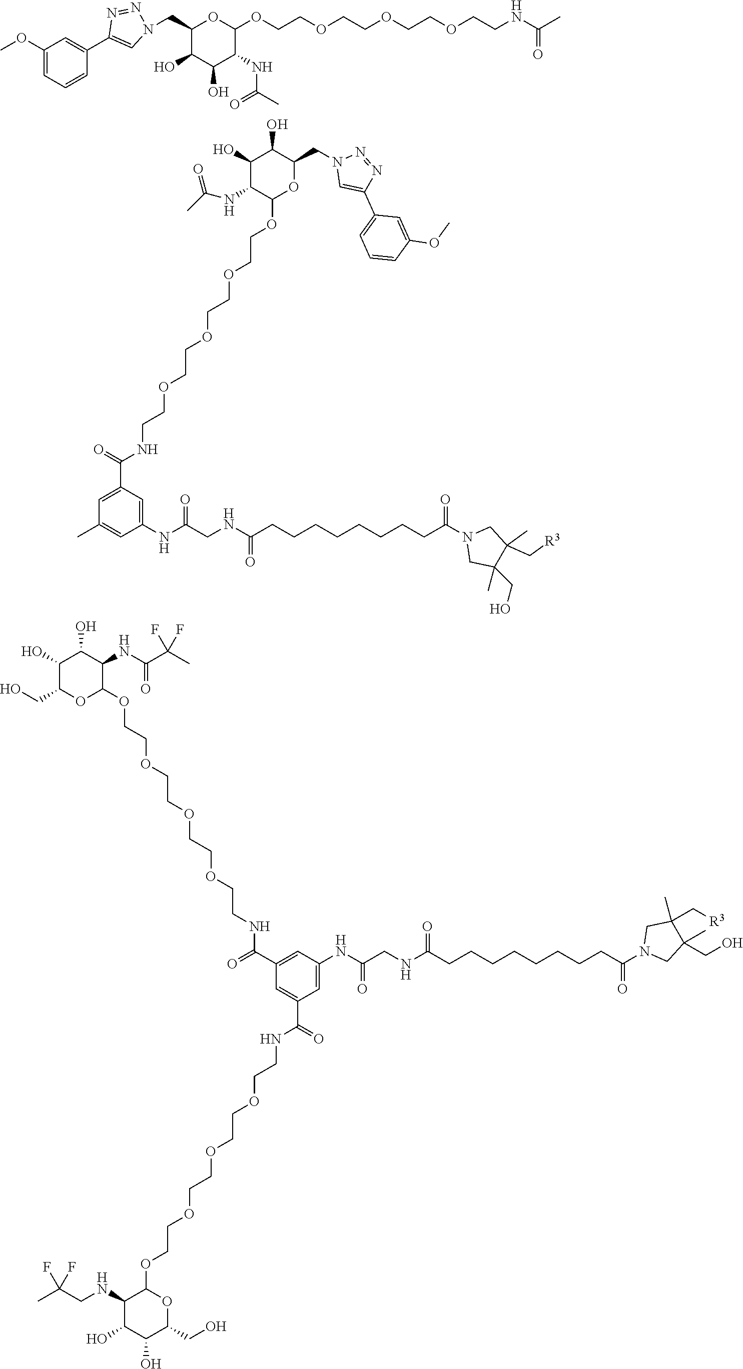

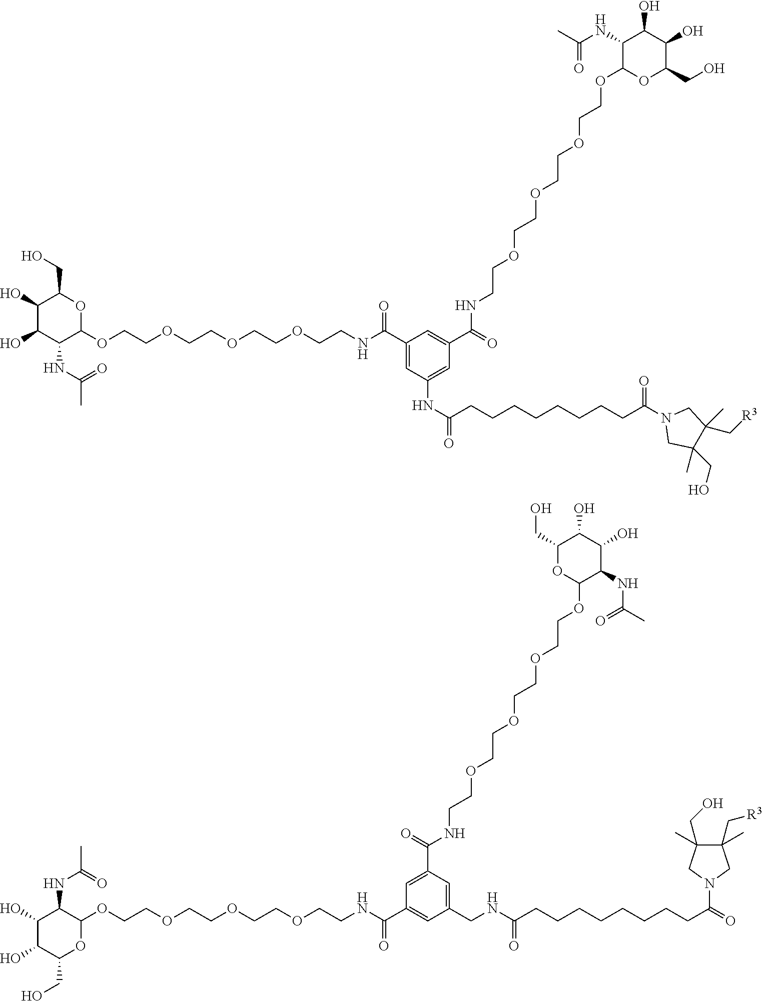

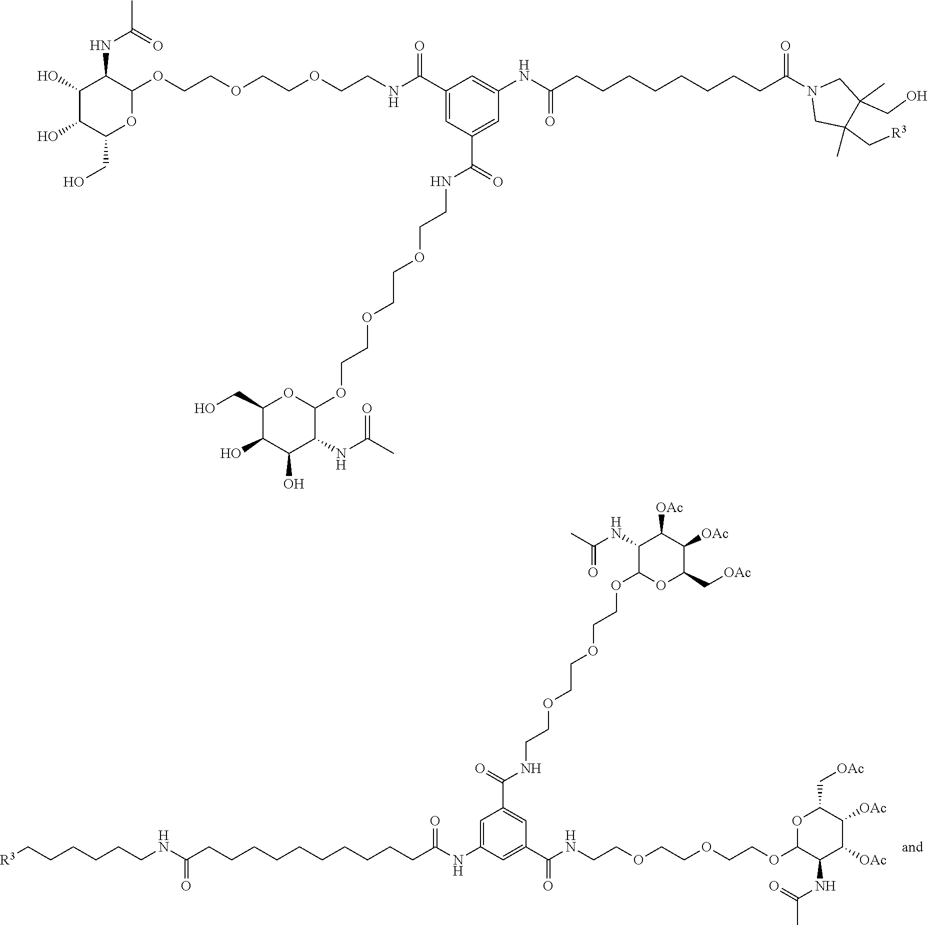

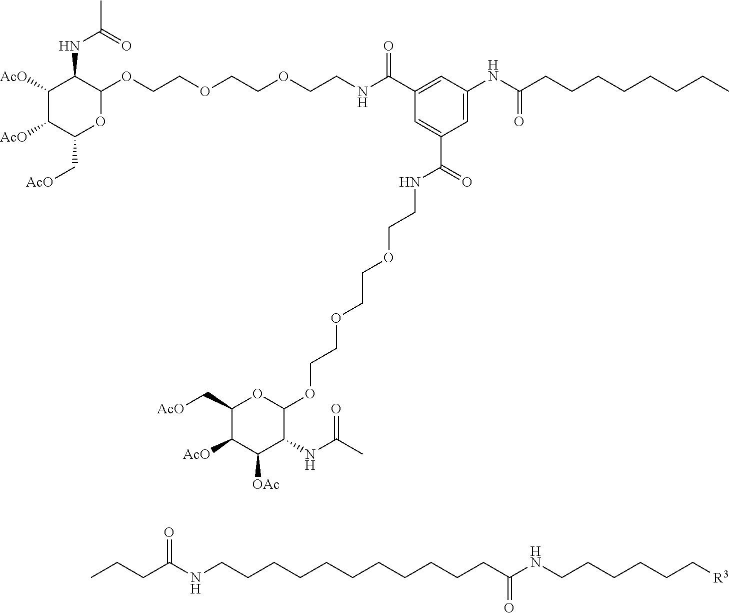

wherein: R³ is a nucleic acid; or a salt thereof.

The term “alkyl”, by itself or as part of another substituent, means, unless otherwise stated, a straight or branched chain hydrocarbon radical, having the number of carbon atoms designated (i.e., C₁₋₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. The term “alkenyl” refers to an unsaturated alkyl radical having one or more double bonds. Similarly, the term “alkynyl” refers to an unsaturated alkyl radical having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane (including straight and branched alkanes), as exemplified by —CH₂CH₂CH₂CH₂— and —CH(CH₃)CH₂CH₂—.

The term “cycloalkyl,” “carbocyclic,” or “carbocycle” refers to hydrocarbon ringsystem having 3 to 20 overall number of ring atoms (e.g., 3-20 membered cycloalkyl is a cycloalkyl with 3 to 20 ring atoms, or C₃₋₂₀ cycloalkyl is a cycloalkyl with 3-20 carbon ring atoms) and for a 3-5 membered cycloalkyl being fully saturated or having no more than one double bond between ring vertices and for a 6 membered cycloalkyl or larger being fully saturated or having no more than two double bonds between ring vertices. As used herein, “cycloalkyl,” “carbocyclic,” or “carbocycle” is also meant to refer to bicyclic, polycyclic and spirocyclic hydrocarbon ring system, such as, for example, bicyclo[2.2.1]heptane, pinane, bicyclo[2.2.2]octane, adamantane, norborene, spirocyclic C₅₋₁₂ alkane, etc. As used herein, the terms, “alkenyl,” “alkynyl,” “cycloalkyl,”, “carbocycle,” and “carbocyclic,” are meant to include mono and polyhalogenated variants thereof.

The term “heterocycloalkyl,” “heterocyclic,” or “heterocycle” refers to a saturated or partially unsaturated ring system radical having the overall having from 3-20 ring atoms (e.g., 3-20 membered heterocycloalkyl is a heterocycloalkyl radical with 3-20 ring atoms, a C₂₋₁₉ heterocycloalkyl is a heterocycloalkyl having 3-10 ring atoms with between 2-19 ring atoms being carbon) that contain from one to ten heteroatoms selected from N, O, and S, wherein the nitrogen and sulfur atoms are optionally oxidized, nitrogen atom(s) are optionally quaternized, as ring atoms. Unless otherwise stated, a “heterocycloalkyl,” “heterocyclic,” or “heterocycle” ring can be a monocyclic, a bicyclic, spirocyclic or a polycylic ring system. Non limiting examples of “heterocycloalkyl,” “heterocyclic,” or “heterocycle” rings include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrhydrothiophene, quinuclidine, tropane, 2-azaspiro[3.3]heptane, (1R,5S)-3-azabicyclo[3.2.1]octane, (1s,4s)-2-azabicyclo[2.2.2]octane, (1R,4R)-2-oxa-5-azabicyclo[2.2.2]octane and the like A “heterocycloalkyl,” “heterocyclic,” or “heterocycle” group can be attached to the remainder of the molecule through one or more ring carbons or heteroatoms. A “heterocycloalkyl,” “heterocyclic,” or “heterocycle” can include mono- and poly-halogenated variants thereof.

The terms “alkoxy,” and “alkylthio”, are used in their conventional sense, and refer to those alkyl groups attached to the remainder of the molecule via an oxygen atom (“oxy”) or thio group, and further include mono- and poly-halogenated variants thereof.

The terms “halo” or “halogen,” by themselves or as part of another substituent, mean, unless otherwise stated, a fluorine, chlorine, bromine, or iodine atom. The term “(halo)alkyl” is meant to include both a “alkyl” and “haloalkyl” substituent. Additionally, the term “haloalkyl,” is meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, difluoromethyl, and the like.

The term “aryl” means a carbocyclic aromatic group having 6-14 carbon atoms, whether or not fused to one or more groups. Examples of aryl groups include phenyl, naphthyl, biphenyl and the like unless otherwise stated.

The term “heteroaryl” refers to aryl ring(s) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like.

The term saccharide includes monosaccharides, disaccharides and trisaccharides. The term includes glucose, sucrose fructose, galactose and ribose, as well as deoxy sugars such as deoxyribose and amino sugar such as galactosamine. Saccharide derivatives can conveniently be prepared as described in International Patent Applications Publication Numbers WO 96/34005 and 97/03995. A saccharide can conveniently be linked to the remainder of the compound through an ether bond, a thioether bond (e.g. an S-glycoside), an amine nitrogen (e.g., an N-glycoside), or a carbon-carbon bond (e.g. a C-glycoside). In one embodiment the saccharide can conveniently be linked to the remainder of a compound through an ether bond. In one embodiment the term saccharide includes a group of the formula:

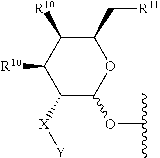

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy. In another embodiment the saccharide can be selected from the group consisting of:

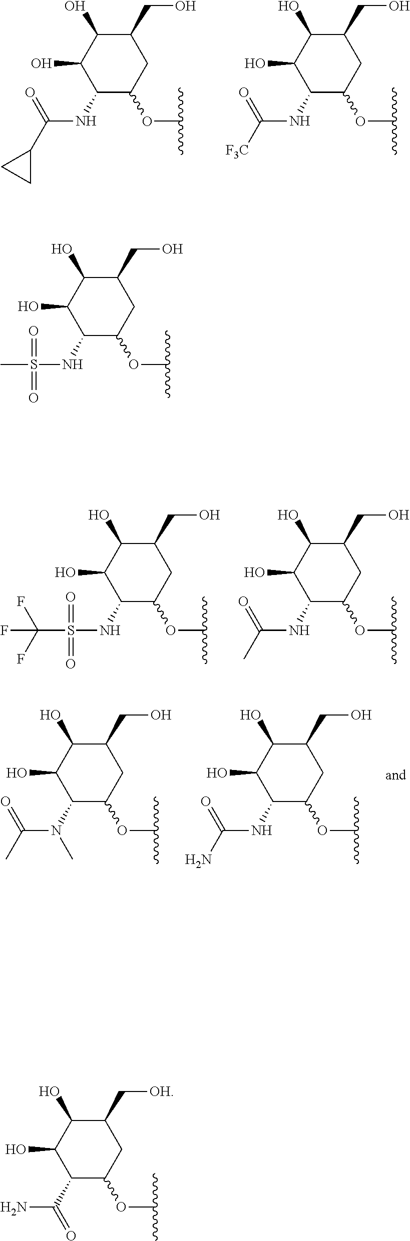

In another embodiment the saccharide can be:

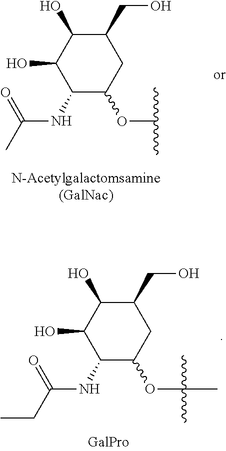 [SMILES: *OC1C[C@H](CO)[C@H](O)[C@H](O)[C@H]1NC(C)=O ; *OC1C[C@H](CO)[C@H](O)[C@H](O)[C@H]1NC(=O)CC]

In certain embodiments, the siRNA of siRNA conjugate is siRNA 1 below. In certain embodiments, the siRNA of the siRNA conjugate is siRNA 2 below. In the experiments described hereinbelow, the siRNA of the siRNA conjugate is siRNA 2 below. An example of an siRNA conjugate is provided below, which in certain embodiments includes siRNA 1 and in other embodiments includes siRNA 2.

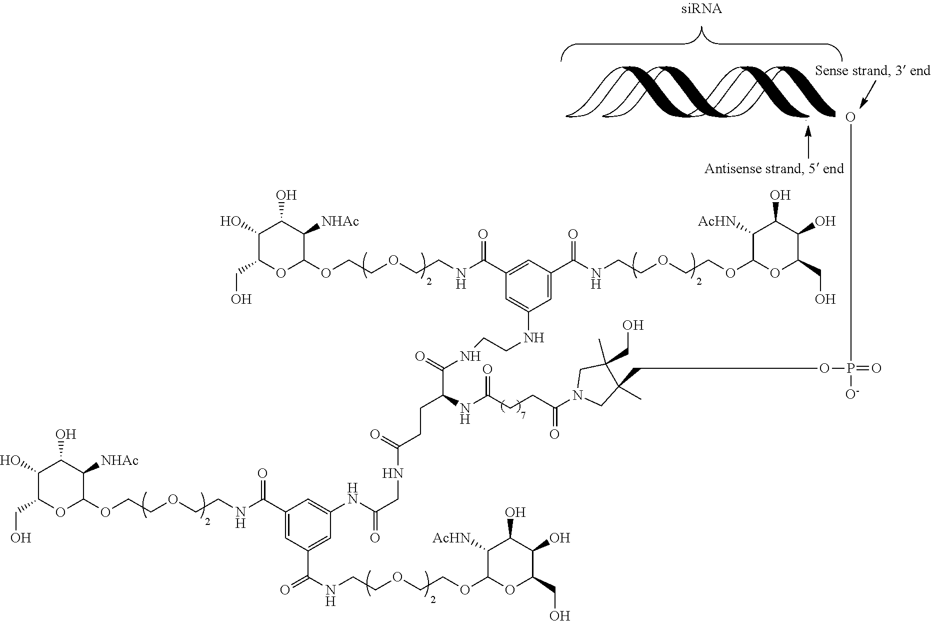

| siRNA Name | Sense Sequence (5′-3′) | Antisense Sequence (5’-3’) |
|---|---|---|
| siRNA 1 | usgscaCUUcgcuucaccu | asGsgugaagcgaagUgCacascsgU |
| siRNA 2 | gsusgcACUucgcuucaca | usGsugaagcgaaguGcAcacsgsgU |

2’-O-Methyl nucleotides = lower case
2’-Fluoro nucleotides = UPPER_CASE
Phosphorothioate linker = s
Unmodified = UPPER CASE

### Oligomeric Nucleotides

The oligomeric nucleotides can be designed to target one or more genes and/or transcripts of the HBV genome. Examples of such siRNA molecules are the siRNA molecules set forth in Table A, B and C herein. In certain embodiments, the siRNA molecules, and combinations thereof, are those described in WO 2016/054421 or in WO 2017/019891.

The term oligomeric nucleotide targeted to the Hepatitis B genome also includes Arrowhead-ARC-520 (see U.S. Pat. No. 8,809,293; and Wooddell C I, et al., Molecular Therapy, 2013, 21, 5, 973-985).

The term oligomeric nucleotide targeted to the Hepatitis B genome also includes isolated, double stranded, siRNA molecules, that each include a sense strand and an antisense strand that is hybridized to the sense strand. The siRNA target one or more genes and/or transcripts of the HBV genome.

The term “Hepatitis B virus” (abbreviated as HBV) refers to a virus species of the genus Orthohepadnavirus, which is a part of the Hepadnaviridae family of viruses, and that is capable of causing liver inflammation in humans.

The term “Hepatitis D virus” (abbreviated as HDV) refers to a virus species of the genus Deltaviridae, which is capable of causing liver inflammation in humans.

The term “small-interfering RNA” or “siRNA” as used herein refers to double stranded RNA (i.e., duplex RNA) that is capable of reducing or inhibiting the expression of a target gene or sequence (e.g., by mediating the degradation or inhibiting the translation of mRNAs which are complementary to the siRNA sequence) when the siRNA is in the same cell as the target gene or sequence. The siRNA may have substantial or complete identity to the target gene or sequence, or may comprise a region of mismatch (i.e., a mismatch motif). In certain embodiments, the siRNAs may be about 19-25 (duplex) nucleotides in length, and is preferably about 20-24, 21-22, or 21-23 (duplex) nucleotides in length. siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand.

Preferably, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Nat. Acad. Sci. USA, 99:9942-9947 (2002); Calegari et al., Proc. Nat. Acad. Sci. USA, 99:14236 (2002); Byrom et al., Ambion TechNotes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

The phrase “inhibiting expression of a target gene” refers to the ability of a siRNA to silence, reduce, or inhibit expression of a target gene (e.g., a gene within the HBV genome). To examine the extent of gene silencing, a test sample (e.g., a biological sample from an organism of interest expressing the target gene or a sample of cells in culture expressing the target gene) is contacted with a siRNA that silences, reduces, or inhibits expression of the target gene. Expression of the target gene in the test sample is compared to expression of the target gene in a control sample (e.g., a biological sample from an organism of interest expressing the target gene or a sample of cells in culture expressing the target gene) that is not contacted with the siRNA. Control samples (e.g., samples expressing the target gene) may be assigned a value of 100%. In particular embodiments, silencing, inhibition, or reduction of expression of a target gene is achieved when the value of the test sample relative to the control sample (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays include, without limitation, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art. An “effective amount” or “therapeutically effective amount” of a therapeutic nucleic acid such as a siRNA is an amount sufficient to produce the desired effect, e.g., an inhibition of expression of a target sequence in comparison to the normal expression level detected in the absence of a siRNA. In particular embodiments, inhibition of expression of a target gene or target sequence is achieved when the value obtained with a siRNA relative to the control (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays for measuring the expression of a target gene or target sequence include, but are not limited to, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid.

Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs). Additionally, nucleic acids can include one or more UNA moieties.

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. RNA may be in the form, for example, of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), and combinations thereof. Accordingly, the terms “polynucleotide” and “oligonucleotide” refer to a polymer or oligomer of nucleotide or nucleoside monomers consisting of naturally-occurring bases, sugars and intersugar (backbone) linkages. The terms “polynucleotide” and “oligonucleotide” also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

An “isolated” or “purified” DNA molecule or RNA molecule is a DNA molecule or RNA molecule that exists apart from its native environment. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The term “unlocked nucleobase analogue” (abbreviated as “UNA”) refers to an acyclic nucleobase in which the C2′ and C3′ atoms of the ribose ring are not covalently linked. The term “unlocked nucleobase analogue” includes nucleobase analogues having the following structure identified as Structure A:

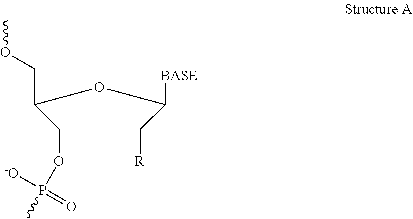

wherein R is hydroxyl, and Base is any natural or unnatural base such as, for example, adenine (A), cytosine (C), guanine (G) and thymine (T). UNA include the molecules identified as acyclic 2′-3′-seco-nucleotide monomers in U.S. patent serial number 8,314,227.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “lipid particle” includes a lipid formulation that can be used to deliver a therapeutic nucleic acid (e.g., siRNA) to a target site of interest (e.g., cell, tissue, organ, and the like). In preferred embodiments, the lipid particle is typically formed from a cationic lipid, a non-cationic lipid, and optionally a conjugated lipid that prevents aggregation of the particle. A lipid particle that includes a nucleic acid molecule (e.g., siRNA molecule) is referred to as a nucleic acid-lipid particle. Typically, the nucleic acid is fully encapsulated within the lipid particle, thereby protecting the nucleic acid from enzymatic degradation.

In certain instances, nucleic acid-lipid particles are extremely useful for systemic applications, as they can exhibit extended circulation lifetimes following intravenous (i.v.) injection, they can accumulate at distal sites (e.g., sites physically separated from the administration site), and they can mediate silencing of target gene expression at these distal sites. The nucleic acid may be complexed with a condensing agent and encapsulated within a lipid particle as set forth in PCT Publication No. WO 00/03683, the disclosure of which is herein incorporated by reference in its entirety for all purposes.

The term “salts” includes any anionic and cationic complex, such as the complex formed between a cationic lipid and one or more anions. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof. In particular embodiments, the salts of the cationic lipids disclosed herein are crystalline salts.

As used herein, the term “aqueous solution” refers to a composition comprising in whole, or in part, water.

“Distal site,” as used herein, refers to a physically separated site, which is not limited to an adjacent capillary bed, but includes sites broadly distributed throughout an organism.

“Serum-stable” in relation to nucleic acid-lipid particles means that the particle is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery of lipid particles that leads to a broad biodistribution of an active agent such as a siRNA within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles can be by any means known in the art including, for example, intravenous, subcutaneous, and intraperitoneal. In a preferred embodiment, systemic delivery of lipid particles is by intravenous delivery.

“Local delivery,” as used herein, refers to delivery of an active agent such as a siRNA directly to a target site within an organism. For example, an agent can be locally delivered by direct injection into a disease site, other target site, or a target organ such as the liver, heart, pancreas, kidney, and the like.

The term “virus particle load”, as used herein, refers to a measure of the number of virus particles (e.g., HBV and/or HDV) present in a bodily fluid, such as blood. For example, particle load may be expressed as the number of virus particles per milliliter of, e.g., blood. Particle load testing may be performed using nucleic acid amplification based tests, as well as non-nucleic acid-based tests (see, e.g., Puren et al., The Journal of Infectious Diseases, 201:S27-36 (2010)).

In certain embodiments, the term “animal” refers to a mammal. The term “mammal” refers to any mammalian species such as a human, mouse, rat, dog, cat, hamster, guinea pig, rabbit, livestock, and the like.

TABLE A

| | | IC 50 |
|---|---|---|
| Name | Duplex Sequences | (nM) |
| 1m | 5’ A g G u A U g u U G C C C g U u U G U U U 3’ | 1.43 |
| | 3’ U U U C C A u A C A A C G G g C A A A C A 5’ | |
| 2m | 5’ G C u c A g U U U A C U A G U G C c A U U 3’ | 0.37 |
| | 3’ U U C g A G U C A A A u G A U C A C G G U 5’ | |
| 3m | 5’ C C G U g u G C A C U u C G C u u C A U U 3’ | 0.06 |
| | 3’ U U G g C A C A C g U G A A G C G A A G U 5’ | |
| 4m | 5’ G C u c A g U U U A C U A G U G C c A U U 3’ | 0.31 |
| | 3’ U U C g A G U C A A A u G A U C A C G G U 5’ | |
| 5m | 5’ C C G U g u G C A C U u C G C u U C A U U 3’ | 0.06 |
| | 3’ U U G g C A C A C g U G A A G C G A A G U 5’ | |
| 6m | 5’ C u g g C U C A G U U U A C u A g U G U U 3’ | 0.05 |
| | 3’ U U G A C C g A g U C A A A U g A U C A C 5’ | |
| 7m | 5’ C C G U g u G C A C U u C H C u U C A U U 3’ | 0.06 |
| | 3’ U U G g C A C A C g U G A A G C G A A G U 5’ | |
| 8m | 5’ G C u C A g U U U A C u A g U G C C A U U 3’ | 0.24 |
| | 3’ U U C G A G u C A A A U G A U C A C G G U 5’ | |
| 9m | 5’ A g G u A U G u U G C C C g U u U G U U U 3’ | 0.13 |
| | 3’ U U u C C A u A C A A C G G g C A A A C A 5 | |
| 10m | 5’ G C C g A u C C A U A C u g C g g A A U U 3’ | 0.34 |
| | 3’ U U C g G C U A g G U A U g A C G C C U U 5’ | |
| 11m | 5’ G C C g A u C C A U A C u g C g g A A U U 3’ | 0.31 |
| | 3’ U U C g G C U A g G U A U g A C G C C U U 5’ | |
| 12m | 5’ G C C g A u C C A U A C u g C G g A A U U 3’ | 0.16 |
| | 3’ U U C g G C U A g G U A U g A C G C C U U 5’ | |
| 13m | 5’ G C C g A u C C A U A C u g C G g A A U U 3’ | 0.2 |
| | 3’ U U C g G C U A g G U A U g A C G C C U U 5’ | |
| 14m | 5’ G C u C A g U U U A C u A g U G C C A U U 3’ | 0.16 |
| | 3’ U U C G A G u C A A A U G A U C A C G G U 5’ | |
| 15m | 5’ C u g G C u C A G U U u A C U A G U G U U 3’ | 0.17 |
| | 3’ U U G A C C g A G U C A A A U G A U C A C 5’ | |

lower case = 2’O-methyl modification
Underline = UNA moiety

The oligonucleotides (such as the sense and antisense RNA strands set forth in Table B) specifically hybridize to or is complementary to a target polynucleotide sequence. The terms “specifically hybridizable” and “complementary” as used herein indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable. In preferred embodiments, an oligonucleotide is specifically hybridizable when binding of the oligonucleotide to the target sequence interferes with the normal function of the target sequence to cause a loss of utility or expression therefrom, and there is a sufficient degree of complementarity to avoid non-specific binding of the oligonucleotide to non-target sequences under conditions in which specific binding is desired, i.e., under physiological conditions in the case of in vivo assays or therapeutic treatment, or, in the case of in vitro assays, under conditions in which the assays are conducted. Thus, the oligonucleotide may include 1, 2, 3, or more base substitutions as compared to the region of a gene or mRNA sequence that it is targeting or to which it specifically hybridizes.

TABLE B

| | Sense Sequence | Antisense Sequence |
|---|---|---|
| Name | (5’-3’) | (5’- 3’) |
| lm | AgGuAUguUGCCCgUuUGUUU | ACAAACgGGCAACAuACCUUU |
| 2m | GCucAgUUUACUAGUGCcAUU | UGGCACUAGuAAACUGAgCUU |
| 3m | CCGUguGCACUuCGCuuCAUU | UGAAGCGAAGUgCACACgGUU |
| 4m | GCucAgUUUACUAGUGCcAUU | UGGCACUAGuAAACUGAgCUU |
| 5m | CCGUguGCACUuCGCuUCAUU | UGAAGCGAAGUgCACACgGUU |
| 6m | CuggCUCAGUUUACuAgUGUU | CACUAgUAAACUgAgCCAGUU |
| 7m | CCGUguGCACUuCGCuUCAUU | UGAAGCGAAGUgCACACgGUU |
| 8m | GCuCAgUUUACuAgUGCCAUU | UGGCACUAGUAAACuGAGCUU |
| 9m | AgGuAUGuUGCCCgUuUGUUU | ACAAACgGGCAACAuACCuUU |
| 10m | GCCgAuCCAUACugCggAAUU | UUCCGCAgUAUGgAUCGgCUU |
| 11m | GCCgAuCCAUACugCggAAUU | UUCCGCAgUAUGgAUCGgCUU |
| 12m | GCCgAuCCAUACugCGgAAUU | UUCCGCAgUAUGgAUCGgCUU |
| 13m | GCCgAuCCAUACugCGgAAUU | UUCCGCAgUAUGgAUCGgCUU |
| 14m | GCuCAgUUUACuAgUGCCAUU | UGGCACUAGUAAACuGAGCUU |
| 15m | CugGCuCAGUUuACUAGUGUU | CACUAGUAAACUGAgCCAGUU |

lower case = 2’O-methyl modification
Underline = UNA moiety

TABLE C

| Name | sense sequence (5′-3′) | antisense sequence (5′-3′) |
|---|---|---|
| 67m | xCrCrGrUmGmUrGrCrArCrUmUrCrGrCmUmUrCrAxUxU | rUrGrArArGrCrGrArArGrUmGrCrArCrArCmGrGxUxU |
| 68m | rCrCmGrUmGmUrGrCrArCrUmUrCmGrCmUmUrCrArUrU | rUrGrArAmGrCmGrArArGmUmGrCrAmCrAmCmGrGrUrU |
| 31m | xCrCmGrUmGmUrGrCrArCrUmUrCmGrCmUmUrCrAxUxU | rUrGrArAmGrCmGrArArGmUmGrCrAmCrAmCmGrGxUxU |
| 69m | rGrCmCmGrAmUrCrCrAmUrArCmUmGrCrGmGrArArUrU | rUrUrCrCrGrCrAmGrUrArUrGmGrArUrCrGmGrCrUrU |
| 70m | xGrCmCmGrAmUrCrCrAmUrArCmUmGrCrGmGrArArUxU | rUrUrCrCrGrCrAmGrUrArUrGmGrArUrCrGmGrCrUrU |
| 71m | xCmUmGmGrCrUrCrArGrUrUrUrArCmUrAmGrUrGrUxU | rCrArCrUrAmGrUrArArArCrUmGrAmGrCrCrArGrUrU |
| 72m | rCmUmGmGrCmUrCrArGmUrUmUrAmCmUrAmGmUmGrUrU | rCrArCrUrAmGmUrArArAmCrUmGrAmGrCmCrArGrUrU |
| 61m | xCmUmGmGrCmUrCrArGmUrUmUrAmCmUrAmGmUmGrUxU | rCrArCrUrAmGmUrArArAmCrUmGrAmGrCmCrArGrUrU |
| 73m | rAmCrCmUrCmUrGmCrCmUrAmArUmCrArUrCrUrCrUrU | rGrArGrArUrGmArUmUrArGrGmCrAmGrAmGrGrUrUrU |
| 21m | xAmCrCmUrCmUrGmCrCmUrAmArUmCrArUrCrUrCxUrU | rGrArGrArUrGmArUmUrArGrGmCrAmGrAmGrGrUxUrU |

rN = RNA of base N
mN = 2'O-methyl modification of base N
xN = unlocked nucleoside analog moiety of base N

In certain embodiments, the therapeutic combination comprises the use of two different double stranded siRNA molecules selected from the group consisting of 1m, 2m, 3m, 4m, 5m, 6m, 7m, 8m, 9m, 10m, 11m, 12m, 13m, 14m, 15m. The two way siRNA combinations of siRNAs 1m thru 15m are: 1m-2m; 1m-3m; 1m-4m; 1m-5m; 1m-6m; 1m-7m; 1m-8m; 1m-9m; 1m-10m; 1m-11m; 1m-12m; 1m-13m; 1m-14m; 1m-15m; 2m-3m; 2m-4m; 2m-5m; 2m-6m; 2m-7m; 2m-8m; 2m-9m; 2m-10m; 2m-11m; 2m-12m; 2m-13m; 2m-14m; 2m-15m; 3m-4m; 3m-5m; 3m-6m; 3m-7m; 3m-8m; 3m-9m; 3m-10m; 3m-11m; 3m-12m; 3m-13m; 3m-14m; 3m-15m; 4m-5m; 4m-6m; 4m-7m; 4m-8m; 4m-9m; 4m-10m; 4m-11m; 4m-12m; 4m-13m; 4m-14m; 4m-15m; 5m-6m; 5m-7m; 5m-8m; 5m-9m; 5m-10m; 5m-11m; 5m-12m; 5m-13m; 5m-14m; 5m-15m; 6m-7m; 6m-8m; 6m-9m; 6m-10m; 6m-11m; 6m-12m; 6m-13m; 6m-14m; 6m-15m; 7m-8m; 7m-9m; 7m-10m; 7m-11m; 7m-12m; 7m-13m; 7m-14m; 7m-15m; 8m-9m; 8m-10m; 8m-11m; 8m-12m; 8m-13m; 8m-14m; 8m-15m; 9m-10m; 9m-11m; 9m-12m; 9m-13m; 9m-14m; 9m-15m; 10m-11m; 10m-12m; 10m-13m; 10m-14m; 10m-15m; 11m-12m; 11m-13m; 11m-14m; 11m-15m; 12m-13m; 12m-14m; 12m-15m; 13m-14m; 13m-15m; and 14m-15m.

In certain embodiments, the therapeutic combination comprises the use of three different double stranded siRNA molecules selected from the group consisting of 1m, 2m, 3m, 4m, 5m, 6m, 7m, 8m, 9m, 10m, 11m, 12m, 13m, 14m, 15m. The three way siRNA combinations of siRNAs 1m thru 15m are: 1m-2m-3m; 1m-2m-4m; 1m-2m-5m; 1m-2m-6m; 1m-2m-7m; 1m-2m-8m; 1m-2m-9m; 1m-2m-10m; 1m-2m-11m; 1m-2m-12m; 1m-2m-13m; 1m-2m-14m; 1m-2m-15m; 1m-3m-4m; 1m-3m-5m; 1m-3m-6m; 1m-3m-7m; 1m-3m-8m; 1m-3m-9m; 1m-3m-10m; 1m-3m-11m; 1m-3m-12m; 1m-3m-13m; 1m-3m-14m; 1m-3m-15m; 1m-4m-5m; 1m-4m-6m; 1m-4m-7m; 1m-4m-8m; 1m-4m-9m; 1m-4m-10m; 1m-4m-11m; 1m-4m-12m; 1m-4m-13m; 1m-4m-14m; 1m-4m-15m; 1m-5m-6m; 1m-5m-7m; 1m-5m-8m; 1m-5m-9m; 1m-5m-10m; 1m-5m-11m; 1m-5m-12m; 1m-5m-13m; 1m-5m-14m; 1m-5m-15m; 1m-6m-7m; 1m-6m-8m; 1m-6m-9m; 1m-6m-10m; 1m-6m-11m; 1m-6m-12m; 1m-6m-13m; 1m-6m-14m; 1m-6m-15m; 1m-7m-8m; 1m-7m-9m; 1m-7m-10m; 1m-7m-11m; 1m-7m-12m; 1m-7m-13m; 1m-7m-14m; 1m-7m-15m; 1m-8m-9m; 1m-8m-10m; 1m-8m-11m; 1m-8m-12m; 1m-8m-13m; 1m-8m-14m; 1m-8m-15m; 1m-9m-10m; 1m-9m-11m; 1m-9m-12m; 1m-9m-13m; 1m-9m-14m; 1m-9m-15m; 1m-10m-11m; 1m-10m-12m; 1m-10m-13m; 1m-10m-14m; 1m-10m-15m; 1m-11m-12m; 1m-11m-13m; 1m-11m-14m; 1m-11m-15m; 1m-12m-13m; 1m-12m-14m; 1m-12m-15m; 1m-13m-14m; 1m-13m-15m; 1m-14m-15m; 2m-3m-4m; 2m-3m-5m; 2m-3m-6m; 2m-3m-7m; 2m-3m-8m; 2m-3m-9m; 2m-3m-10m; 2m-3m-11m; 2m-3m-12m; 2m-3m-13m; 2m-3m-14m; 2m-3m-15m; 2m-4m-5m; 2m-4m-6m; 2m-4m-7m; 2m-4m-8m; 2m-4m-9m; 2m-4m-10m; 2m-4m-11m; 2m-4m-12m; 2m-4m-13m; 2m-4m-14m; 2m-4m-15m; 2m-5m-6m; 2m-5m-7m; 2m-5m-8m; 2m-5m-9m; 2m-5m-10m; 2m-5m-11m; 2m-5m-12m; 2m-5m-13m; 2m-5m-14m; 2m-5m-15m; 2m-6m-7m; 2m-6m-8m; 2m-6m-9m; 2m-6m-10m; 2m-6m-11m; 2m-6m-12m; 2m-6m-13m; 2m-6m-14m; 2m-6m-15m; 2m-7m-8m; 2m-7m-9m; 2m-7m-10m; 2m-7m-11m; 2m-7m-12m; 2m-7m-13m; 2m-7m-14m; 2m-7m-15m; 2m-8m-9m; 2m-8m-10m; 2m-8m-11m; 2m-8m-12m; 2m-8m-13m; 2m-8m-14m; 2m-8m-15m; 2m-9m-10m; 2m-9m-11m; 2m-9m-12m; 2m-9m-13m; 2m-9m-14m; 2m-9m-15m; 2m-10m-11m; 2m-10m-12m; 2m-10m-13m; 2m-10m-14m; 2m-10m-15m; 2m-11m-12m; 2m-11m-13m; 2m-11m-14m; 2m-11m-15m; 2m-12m-13m; 2m-12m-14m; 2m-12m-15m; 2m-13m-14m; 2m-13m-15m; 2m-14m-15m; 3m-4m-5m; 3m-4m-6m; 3m-4m-7m; 3m-4m-8m; 3m-4m-9m; 3m-4m-10m; 3m-4m-11m; 3m-4m-12m; 3m-4m-13m; 3m-4m-14m; 3m-4m-15m; 3m-5m-6m; 3m-5m-7m; 3m-5m-8m; 3m-5m-9m; 3m-5m-10m; 3m-5m-11m; 3m-5m-12m; 3m-5m-13m; 3m-5m-14m; 3m-5m-15m; 3m-6m-7m; 3m-6m-8m; 3m-6m-9m; 3m-6m-10m; 3m-6m-11m; 3m-6m-12m; 3m-6m-13m; 3m-6m-14m; 3m-6m-15m; 3m-7m-8m; 3m-7m-9m; 3m-7m-10m; 3m-7m-11m; 3m-7m-12m; 3m-7m-13m; 3m-7m-14m; 3m-7m-15m; 3m-8m-9m; 3m-8m-10m; 3m-8m-11m; 3m-8m-12m; 3m-8m-13m; 3m-8m-14m; 3m-8m-15m; 3m-9m-10m; 3m-9m-11m; 3m-9m-12m; 3m-9m-13m; 3m-9m-14m; 3m-9m-15m; 3m-10m-11m; 3m-10m-12m; 3m-10m-13m; 3m-10m-14m; 3m-10m-15m; 3m-11m-12m; 3m-11m-13m; 3m-11m-14m; 3m-11m-15m; 3m-12m-13m; 3m-12m-14m; 3m-12m-15m; 3m-13m-14m; 3m-13m-15m; 3m-14m-15m; 4m-5m-6m; 4m-5m-7m; 4m-5m-8m; 4m-5m-9m; 4m-5m-10m; 4m-5m-11m; 4m-5m-12m; 4m-5m-13m; 4m-5m-14m; 4m-5m-15m; 4m-6m-7m; 4m-6m-8m; 4m-6m-9m; 4m-6m-10m; 4m-6m-11m; 4m-6m-12m; 4m-6m-13m; 4m-6m-14m; 4m-6m-15m; 4m-7m-8m; 4m-7m-9m; 4m-7m-10m; 4m-7m-11m; 4m-7m-12m; 4m-7m-13m; 4m-7m-14m; 4m-7m-15m; 4m-8m-9m; 4m-8m-10m; 4m-8m-11m; 4m-8m-12m; 4m-8m-13m; 4m-8m-14m; 4m-8m-15m; 4m-9m-10m; 4m-9m-11m; 4m-9m-12m; 4m-9m-13m; 4m-9m-14m; 4m-9m-15m; 4m-10m-11m; 4m-10m-12m; 4m-10m-13m; 4m-10m-14m; 4m-10m-15m; 4m-11m-12m; 4m-11m-13m; 4m-11m-14m; 4m-11m-15m; 4m-12m-13m; 4m-12m-14m; 4m-12m-15m; 4m-13m-14m; 4m-13m-15m; 4m-14m-15m; 5m-6m-7m; 5m-6m-8m; 5m-6m-9m; 5m-6m-10m; 5m-6m-11m; 5m-6m-12m; 5m-6m-13m; 5m-6m-14m; 5m-6m-15m; 5m-7m-8m; 5m-7m-9m; 5m-7m-10m; 5m-7m-11m; 5m-7m-12m; 5m-7m-13m; 5m-7m-14m; 5m-7m-15m; 5m-8m-9m; 5m-8m-10m; 5m-8m-11m; 5m-8m-12m; 5m-8m-13m; 5m-8m-14m; 5m-8m-15m; 5m-9m-10m; 5m-9m-11m; 5m-9m-12m; 5m-9m-13m; 5m-9m-14m; 5m-9m-15m; 5m-10m-11m; 5m-10m-12m; 5m-10m-13m; 5m-10m-14m; 5m-10m-15m; 5m-11m-12m; 5m-11m-13m; 5m-11m-14m; 5m-11m-15m; 5m-12m-13m; 5m-12m-14m; 5m-12m-15m; 5m-13m-14m; 5m-13m-15m; 5m-14m-15m; 6m-7m-8m; 6m-7m-9m; 6m-7m-10m; 6m-7m-11m; 6m-7m-12m; 6m-7m-13m; 6m-7m-14m; 6m-7m-15m; 6m-8m-9m; 6m-8m-10m; 6m-8m-11m; 6m-8m-12m; 6m-8m-13m; 6m-8m-14m; 6m-8m-15m; 6m-9m-10m; 6m-9m-11m; 6m-9m-12m; 6m-9m-13m; 6m-9m-14m; 6m-9m-15m; 6m-10m-11m; 6m-10m-12m; 6m-10m-13m; 6m-10m-14m; 6m-10m-15m; 6m-11m-12m; 6m-11m-13m; 6m-11m-14m; 6m-11m-15m; 6m-12m-13m; 6m-12m-14m; 6m-12m-15m; 6m-13m-14m; 6m-13m-15m; 6m-14m-15m; 7m-8m-9m; 7m-8m-10m; 7m-8m-11m; 7m-8m-12m; 7m-8m-13m; 7m-8m-14m; 7m-8m-15m; 7m-9m-10m; 7m-9m-11m; 7m-9m-12m; 7m-9m-13m; 7m-9m-14m; 7m-9m-15m; 7m-10m-11m; 7m-10m-12m; 7m-10m-13m; 7m-10m-14m; 7m-10m-15m; 7m-11m-12m; 7m-11m-13m; 7m-11m-14m; 7m-11m-15m; 7m-12m-13m; 7m-12m-14m; 7m-12m-15m; 7m-13m-14m; 7m-13m-15m; 7m-14m-15m; 8m-9m-10m; 8m-9m-11m; 8m-9m-12m; 8m-9m-13m; 8m-9m-14m; 8m-9m-15m; 8m-10m-11m; 8m-10m-12m; 8m-10m-13m; 8m-10m-14m; 8m-10m-15m; 8m-11m-12m; 8m-11m-13m; 8m-11m-14m; 8m-11m-15m; 8m-12m-13m; 8m-12m-14m; 8m-12m-15m; 8m-13m-14m; 8m-13m-15m; 8m-14m-15m; 9m-10m-11m; 9m-10m-12m; 9m-10m-13m; 9m-10m-14m; 9m-10m-15m; 9m-11m-12m; 9m-11m-13m; 9m-11m-14m; 9m-11m-15m; 9m-12m-13m; 9m-12m-14m; 9m-12m-15m; 9m-13m-14m; 9m-13m-15m; 9m-14m-15m; 10m-11m-12m; 10m-11m-13m; 10m-11m-14m; 10m-11m-15m; 10m-12m-13m; 10m-12m-14m; 10m-12m-15m; 10m-13m-14m; 10m-13m-15m; 10m-14m-15m; 11m-12m-13m; 11m-12m-14m; 11m-12m-15m; 11m-13m-14m; 11m-13m-15m; 11m-14m-15m; 12m-13m-14m; 12m-13m-15m; 12m-14m-15m; and 13m-14m-15m.

Other combinations of three different siRNA include, for example, 67m-68m-69m, 67m-68m-73m, 67m-69m-71m, 67m-70m-73m, 67m-71m-73m, 67m-72m-73m, 68m-69m-70m, 68m-69m-73m, 68m-70m-72m, 68m-71m-73m; 68m-72m-73m, 69m-70m-71m, 69m-70m-73m, 69m-71m-73m, 69m-72m-73m, 70m-71m-72m, 70m-71m-73m, 70m-72m-73m, 71m-72m-73m.

Generating siRNA Molecules

siRNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. In some embodiments, siRNA may be produced enzymatically or by partial/total organic synthesis, and modified ribonucleotides can be introduced by in vitro enzymatic or organic synthesis. In certain instances, each strand is prepared chemically. Methods of synthesizing RNA molecules are known in the art, e.g., the chemical synthesis methods as described in Verma and Eckstein (1998) or as described herein.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994). The disclosures of these references are herein incorporated by reference in their entirety for all purposes.

Typically, siRNA are chemically synthesized. The oligonucleotides that comprise the siRNA molecules can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol. Alternatively, syntheses at the 0.2 μmol scale can be performed on a 96-well plate synthesizer from Protogene (Palo Alto, Calif.). However, a larger or smaller scale of synthesis is also within the scope. Suitable reagents for oligonucleotide synthesis, methods for RNA deprotection, and methods for RNA purification are known to those of skill in the art.

siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection.

### Carrier Systems Containing Therapeutic Nucleic Acids

Lipid Particles

The lipid particles can comprise one or more siRNA (e.g., siRNA molecules described in Table A, B or C), a cationic lipid, a non-cationic lipid, and a conjugated lipid that inhibits aggregation of particles. In some embodiments, the siRNA molecule is fully encapsulated within the lipid portion of the lipid particle such that the siRNA molecule in the lipid particle is resistant in aqueous solution to nuclease degradation. In other embodiments, the lipid particles described herein are substantially non-toxic to mammals such as humans.

The siRNA two-way and three-way combinations are useful, for example, to treat HBV and/or HDV infection in humans, and to ameliorate at least one symptom associated with the HBV infection and/or HDV infection.

In certain embodiments, with respect to methods that include the use of a cocktail of siRNAs encapsulated within lipid particles, the different siRNA molecules are co-encapsulated in the same lipid particle.

In certain embodiments, the with respect to methods that include the use of a cocktail of siRNAs encapsulated within lipid particles, each type of siRNA species present in the cocktail is encapsulated in its own particle.

In certain embodiments, the with respect to methods that include the use of a cocktail of siRNAs encapsulated within lipid particles, some siRNA species are coencapsulated in the same particle while other siRNA species are encapsulated in different particles.

### Formulation and Administration of Two or More Agents

It will be understood that the agents can be formulated together in a single preparation or that they can be formulated separately and, thus, administered separately, either simultaneously or sequentially. In one embodiment, when the agents are administered sequentially (e.g. at different times), the agents may be administered so that their biological effects overlap (i.e. each agent is producing a biological effect at a single given time).

The agents can be formulated for and administered using any acceptable route of administration depending on the agent selected. For example, suitable routes include, but are not limited to, oral, sublingual, buccal, topical, transdermal, parenteral, subcutaneous, intraperitoneal, intrapulmonary, and intranasal, and, if desired for local treatment, intralesional administration. In one embodiment, the small molecule agents identified herein can be administered orally. In another embodiment, the oligomeric nucleotides can be administered by injection (e.g., into a blood vessel, such as a vein), or subcutaneously. In some embodiments, a subject in need thereof is administered one or more agent orally (e.g., in pill form), and also one or more oligomeric nucleotides by injection or subcutaneously.

Typically, the oligomeric nucleotides targeted to the Hepatitis B genome are administered intravenously, for example in a lipid nanoparticle formulation, however, the present invention is not limited to intravenous formulations comprising the oligomeric nucleotides or to treatment methods wherein an oligomeric nucleotides is administered intravenously.

The agents can be individually formulated by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may typically range anywhere from about 3 to about 8. The agents ordinarily will be stored as a solid composition, although lyophilized formulations or aqueous solutions are acceptable.

Compositions comprising the agents can be formulated, dosed, and administered in a fashion consistent with good medical practice. Factors for consideration in this context include the particular disorder being treated, the particular mammal being treated, the clinical condition of the individual patient, the cause of the disorder, the site of administration, the method of administration, the scheduling of administration, and other factors known to medical practitioners.

The agents may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents. If parenteral administration is desired, the compositions will be sterile and in a solution or suspension form suitable for injection or infusion.

Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug or aid in the manufacturing of the pharmaceutical product (i.e., medicament).

The agents are typically dosed at least at a level to reach the desired biological effect. Thus, an effective dosing regimen will dose at least a minimum amount that reaches the desired biological effect, or biologically effective dose, however, the dose should not be so high as to outweigh the benefit of the biological effect with unacceptable side effects. Therefore, an effective dosing regimen will dose no more than the maximum tolerated dose (“MTD”). The maximum tolerated dose is defined as the highest dose that produces an acceptable incidence of dose-limiting toxicities (“DLT”). Doses that cause an unacceptable rate of DLT are considered non-tolerated. Typically, the MTD for a particular schedule is established in phase 1 clinical trials. These are usually conducted in patients by starting at a safe starting dose of 1/10 the severe toxic dose (“STD10”) in rodents (on a mg/m² basis) and accruing patients in cohorts of three, escalating the dose according to a modified Fibonacci sequence in which ever higher escalation steps have ever decreasing relative increments (e.g., dose increases of 100%, 65%, 50%, 40%, and 30% to 35% thereafter). The dose escalation is continued in cohorts of three patients until a non-tolerated dose is reached. The next lower dose level that produces an acceptable rate of DLT is considered to be the MTD.

The amount of the agents administered will depend upon the particular agent used, the strain of HBV being treated, the age, weight, and condition of the patient, and the judgment of the clinician, but will generally be between about 0.2 to 2.0 grams per day.

### Kits

One embodiment provides a kit. The kit may comprise a container comprising the combination. Suitable containers include, for example, bottles, vials, syringes, blister pack, etc. The container may be formed from a variety of materials such as glass or plastic. The container may hold the combination which is effective for treating the condition and may have a sterile access port (for example, the container may be an intravenous solution bag or a vial having a stopper pierceable by a hypodermic injection needle).

The kit may further comprise a label or package-insert on or associated with the container. The term “package-insert” is used to refer to instructions customarily included in commercial packages of therapeutic agents that contain information about the indications, usage, dosage, administration, contraindications and/or warnings concerning the use of such therapeutic agents. In one embodiment, the label or package inserts indicates that the therapeutic agents can be used to treat a viral infection, such as Hepatitis B.

In certain embodiments, the kits are suitable for the delivery of solid oral forms of the therapeutic agents, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to another embodiment, a kit may comprise (a) a first container with one agent contained therein; and (b) a second container with a second agent contained therein. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the therapeutic agents. For example, the kit may further comprise directions for the simultaneous, sequential or separate administration of the therapeutic agents to a patient in need thereof.

In certain other embodiments, the kit may comprise a container for containing separate compositions such as a divided bottle or a divided foil packet, however, the separate compositions may also be contained within a single, undivided container. In certain embodiments, the kit comprises directions for the administration of the separate therapeutic agents. The kit form is particularly advantageous when the separate therapeutic agents are preferably administered in different dosage forms (e.g., oral and parenteral), are administered at different dosage intervals, or when titration of the individual therapeutic agents of the combination is desired by the prescribing physician.

### Certain Embodiments

In one embodiment, the methods of the invention exclude a method for treating hepatitis B in an animal comprising administering to the animal a synergistically effective amount of i) a formation inhibitor of covalently closed circular DNA and ii) a nucleoside or nucleotide analog.

In one embodiment, the pharmaceutical compositions of the invention exclude compositions comprising, i) a formation inhibitor of covalently closed circular DNA and ii) a nucleoside or nucleotide analog as the only active hepatitis B therapeutic agents.

In one embodiment, the kits of the invention exclude kits comprising, i) a formation inhibitor of covalently closed circular DNA and ii) a nucleoside or nucleotide analog as the only hepatitis B agents.

In one embodiment, the methods of the invention exclude a method for treating hepatitis B in an animal comprising administering to the animal i) one or more siRNA that target a hepatitis B virus and ii) a reverse transcriptase inhibitor.

In one embodiment, the pharmaceutical compositions of the invention exclude compositions comprising, i) one or more siRNA that target a hepatitis B virus and ii) a reverse transcriptase inhibitor as the only active hepatitis B therapeutic agents.

In one embodiment, the kits of the invention exclude kits comprising, i) one or more siRNA that target a hepatitis B virus and ii) a reverse transcriptase inhibitor as the only hepatitis B agents.

In one embodiment the invention provides a method for treating hepatitis B in an animal comprising administering to the animal, at least two agents selected from the group consisting of:
a) reverse transcriptase inhibitors;
b) capsid inhibitors;
c) cccDNA formation inhibitors;
d) sAg secretion inhibitors;
e) oligomeric nucleotides targeted to the Hepatitis B genome; and
f) immunostimulators.

In one embodiment the invention provides a method for treating hepatitis B in an animal comprising administering to the animal, at least two agents selected from the group consisting of:
a) reverse transcriptase inhibitors;
b) capsid inhibitors;
c) cccDNA formation inhibitors;
d) sAg secretion inhibitors; and
e) immunostimulators.

In one embodiment the invention provides a combination of at least two agents selected from the group consisting of:
a) reverse transcriptase inhibitors;
b) capsid inhibitors;
c) cccDNA formation inhibitors;
d) sAg secretion inhibitors;
e) oligomeric nucleotides targeted to the Hepatitis B genome; and
f) immunostimulators
   for use in treating hepatitis B in an animal.

As used herein, the term “a combination” refers to the simultaneous or sequential administration of the at least two agents. For simultaneous administration, the at least two agents may be present in a single composition or may be separate (e.g., may be administered by the same or different routes).

In one embodiment the invention provides a combination of at least two agents selected from the group consisting of:
a) reverse transcriptase inhibitors;
b) capsid inhibitors;
c) cccDNA formation inhibitors;
d) sAg secretion inhibitors; and
e) immunostimulators,
   for use in treating hepatitis B in an animal.

In one embodiment the invention provides the use of a combination of at least two agents selected from the group consisting of:
a) reverse transcriptase inhibitors;
b) capsid inhibitors;
c) cccDNA formation inhibitors;
d) sAg secretion inhibitors;
e) oligomeric nucleotides targeted to the Hepatitis B genome; and
f) immunostimulators
   in the manufacture of a medicament for the treatment of Hepatitis B in an animal.

In one embodiment the invention provides the use of a combination of at least two agents selected from the group consisting of:
a) reverse transcriptase inhibitors;
b) capsid inhibitors;
c) cccDNA formation inhibitors;
d) sAg secretion inhibitors; and
e) immunostimulators,
   in the manufacture of a medicament for the treatment of Hepatitis B in an animal.

In one embodiment the invention provides a method for treating hepatitis D in an animal comprising administering to the animal, at least two agents selected from the group consisting of:
a) reverse transcriptase inhibitors;
b) capsid inhibitors;
c) cccDNA formation inhibitors;
d) sAg secretion inhibitors;
e) oligomeric nucleotides targeted to the Hepatitis B genome; and
f) immunostimulators.

In one embodiment the invention provides a method for treating hepatitis D in an animal comprising administering to the animal, at least two agents selected from the group consisting of:
a) reverse transcriptase inhibitors;
b) capsid inhibitors;
c) cccDNA formation inhibitors;
d) sAg secretion inhibitors; and
e) immunostimulators.

In another embodiment the invention provides a kit comprising at least two agents selected from the group consisting of:
a) reverse transcriptase inhibitors;
b) capsid inhibitors;
c) cccDNA formation inhibitors;
d) sAg secretion inhibitors;
e) oligomeric nucleotides targeted to the Hepatitis B genome; and
f) immunostimulators. In certain embodiments, the kit is for use in combination to treat or prevent a viral infection, such as Hepatitis B. In certain embodiments, the kit is for use in combination to treat or prevent a viral infection, such as Hepatitis D.

In one embodiment the invention provides a kit comprising at least two agents selected from the group consisting of:
a) reverse transcriptase inhibitors;
b) capsid inhibitors;
c) cccDNA formation inhibitors;
d) sAg secretion inhibitors; and
e) immunostimulators.

In another embodiment the invention provides a pharmaceutical composition that comprises a pharmaceutically acceptable carrier and at least two agents selected from the group consisting of:
a) reverse transcriptase inhibitors;
b) capsid inhibitors;
c) cccDNA formation inhibitors;
d) sAg secretion inhibitors;
e) oligomeric nucleotides targeted to the Hepatitis B genome; and
f) immunostimulators.

In another embodiment the invention provides a pharmaceutical composition that comprises a pharmaceutically acceptable carrier and at least two agents selected from the group consisting of:
a) reverse transcriptase inhibitors;
b) capsid inhibitors;
c) cccDNA formation inhibitors;
d) sAg secretion inhibitors; and
e) immunostimulators.

In one embodiment the invention provides a method for treating hepatitis B in an animal comprising administering to the animal, an oligomeric nucleotide targeted to the Hepatitis B genome and at least one additional agent selected from the group consisting of:
a) reverse transcriptase inhibitors;
b) capsid inhibitors;
c) cccDNA formation inhibitors;
d) sAg secretion inhibitors; and
e) immunostimulators.

In one embodiment the invention provides a pharmaceutical composition comprising an oligomeric nucleotide targeted to the Hepatitis B genome and at least one additional agent selected from the group consisting of:
a) reverse transcriptase inhibitors;
b) capsid inhibitors;
c) cccDNA formation inhibitors;
d) sAg secretion inhibitors; and
e) immunostimulators.

In one embodiment the invention provides a kit comprising an oligomeric nucleotide targeted to the Hepatitis B genome and at least one additional agent selected from the group consisting of:
a) reverse transcriptase inhibitors;
b) capsid inhibitors;
c) cccDNA formation inhibitors;
d) sAg secretion inhibitors; and
e) immunostimulators.

Certain embodiments of the invention provide a pharmaceutical composition that comprises a pharmaceutically acceptable carrier and at least two agents selected from the group consisting of:

a) a capsid inhibitor, wherein the capsid inhibitor is:

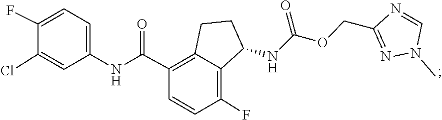 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

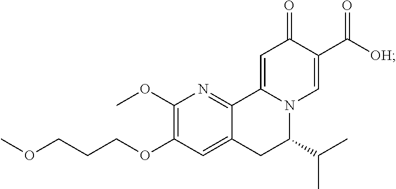 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and

d) oligomeric nucleotides targeted to the Hepatitis B genome.

Certain embodiments of the invention provide a pharmaceutical composition that comprises a pharmaceutically acceptable carrier and at least two agents selected from the group consisting of:

a) a capsid inhibitor, wherein the capsid inhibitor is:

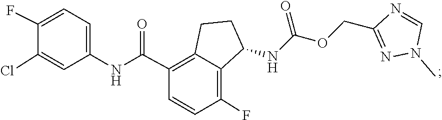 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

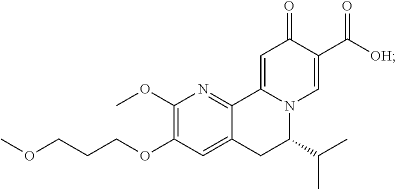 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and

d) oligomeric nucleotides targeted to the Hepatitis B genome;

provided that at least one of the agents in the pharmaceutical composition is the capsid inhibitor or the RNA destabilizer.

Certain embodiments of the invention provide a pharmaceutical composition that comprises a pharmaceutically acceptable carrier and at least two agents selected from the group consisting of:

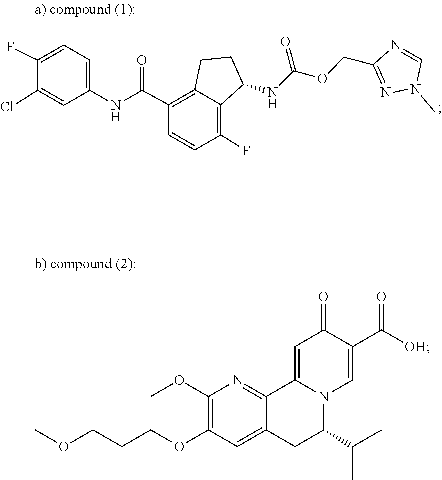

c) a compound selected from the group consisting of tenofovir disoproxil fumarate, tenofovir alafenamide and entecavir; and

d) oligomeric nucleotides targeted to the Hepatitis B genome.

In certain embodiments, the pharmaceutical composition comprises at least three oligomeric nucleotides targeted to the Hepatitis B genome. In certain embodiments, the pharmaceutical composition comprises oligomeric nucleotides 3m, 6m and 12m as described herein. In certain embodiments, the oligomeric nucleotides are comprised within a lipid nanoparticle formulation.

In certain embodiments, the pharmaceutical composition comprises one of the following combinations of two agents:

the RNA destabilizer and the capsid inhibitor;
at least one oligomeric nucleotide targeted to the Hepatitis B genome and the capsid inhibitor;
at least one oligomeric nucleotide targeted to the Hepatitis B genome and the RNA destabilizer;
at least one oligomeric nucleotide targeted to the Hepatitis B genome and a reverse transcriptase inhibitor;
the capsid inhibitor and a reverse transcriptase inhibitor; or
the RNA destabilizer and a reverse transcriptase inhibitor.

In certain embodiments, the pharmaceutical composition comprises one of the following combinations of two agents:

the RNA destabilizer and the capsid inhibitor;
a combination of oligomeric nucleotides 3m, 6m and 12m and the capsid inhibitor;
the capsid inhibitor and tenofovir disoproxil fumarate;
the capsid inhibitor and tenofovir alafenamide;
the capsid inhibitor and entecavir;
the RNA destabilizer and tenofovir disoproxil fumarate;
the RNA destabilizer and tenofovir alafenamide; or
the RNA destabilizer and entecavir.

In certain embodiments, the pharmaceutical composition comprises the RNA destabilizer (compound 2) and the capsid inhibitor (compound 1).

In certain embodiments, the pharmaceutical composition comprises a combination of oligomeric nucleotides 3m, 6m and 12m and the capsid inhibitor (compound 1).

In certain embodiments, the pharmaceutical composition comprises the capsid inhibitor (compound 1) and tenofovir disoproxil fumarate.

In certain embodiments, the pharmaceutical composition comprises the capsid inhibitor (compound 1) and tenofovir alafenamide.

In certain embodiments, the pharmaceutical composition comprises the capsid inhibitor (compound 1) and entecavir.

In certain embodiments, the pharmaceutical composition comprises the RNA destabilizer (compound 2) and tenofovir disoproxil fumarate.

In certain embodiments, the pharmaceutical composition comprises the RNA destabilizer (compound 2) and tenofovir alafenamide.

In certain embodiments, the pharmaceutical composition comprises the RNA destabilizer (compound 2) and entecavir.

In certain embodiments, the pharmaceutical composition comprises one of the following combinations of three agents:

the capsid inhibitor, the RNA destabilizer and a reverse transcriptase inhibitor;
the capsid inhibitor, at least one oligomeric nucleotide targeted to the Hepatitis B genome and a reverse transcriptase inhibitor;
the capsid inhibitor, the RNA destabilizer and at least one oligomeric nucleotide targeted to the Hepatitis B genome; or
the RNA destabilizer, at least one oligomeric nucleotide targeted to the Hepatitis B genome and a reverse transcriptase inhibitor.

In certain embodiments, the pharmaceutical composition comprises one of the following combinations of three agents:

the capsid inhibitor, the RNA destabilizer and tenofovir disoproxil fumarate;
the capsid inhibitor, the RNA destabilizer and tenofovir alafenamide; or
the capsid inhibitor, the RNA destabilizer and entecavir.

In certain embodiments, the pharmaceutical composition comprises the capsid inhibitor (compound 1), the RNA destabilizer (compound 2) and tenofovir disoproxil fumarate.

In certain embodiments, the pharmaceutical composition comprises the capsid inhibitor (compound 1), the RNA destabilizer (compound 2) and tenofovir alafenamide.

In certain embodiments, the pharmaceutical composition comprises the capsid inhibitor (compound 1), the RNA destabilizer (compound 2) and entecavir.

Certain embodiments of the invention provide a kit comprising at least two agents selected from the group consisting of:
a) a capsid inhibitor, wherein the capsid inhibitor is:

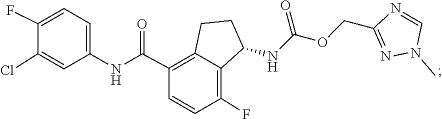 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

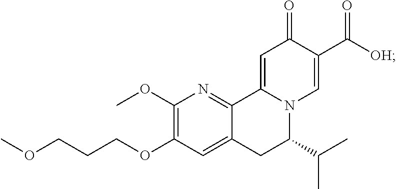 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and
d) oligomeric nucleotides targeted to the Hepatitis B genome;
   for use in combination to treat or prevent a viral infection, such as Hepatitis B.

Certain embodiments of the invention provide a kit comprising at least two agents selected from the group consisting of:
a) a capsid inhibitor, wherein the capsid inhibitor is:

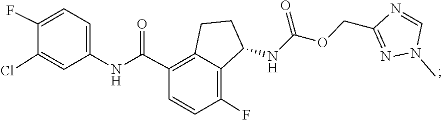 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

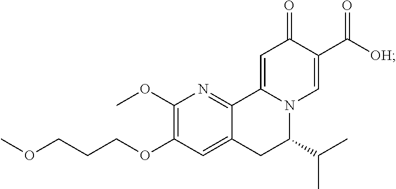 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and
d) oligomeric nucleotides targeted to the Hepatitis B genome;
provided that at least one agent in the kits is the capsid inhibitor or the RNA destabilizer, for use in combination to treat or prevent a viral infection, such as Hepatitis B.

Certain embodiments of the invention provide a kit comprising at least two agents selected from the group consisting of:

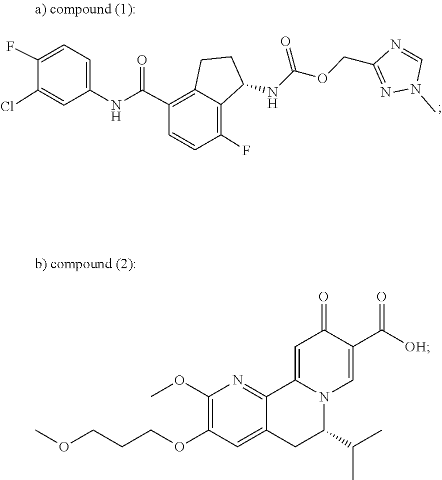

c) a compound selected from the group consisting of tenofovir disoproxil fumarate, tenofovir alafenamide and entecavir; and
d) oligomeric nucleotides targeted to the Hepatitis B genome;
   for use in combination to treat or prevent a viral infection, such as Hepatitis B.

Certain embodiments of the invention provide a kit comprising at least two agents selected from the group consisting of:
a) a capsid inhibitor, wherein the capsid inhibitor is:

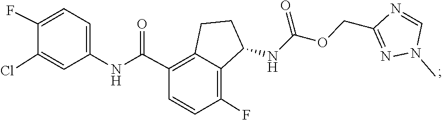 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

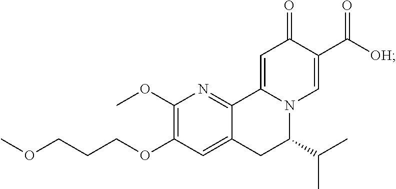 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and
d) oligomeric nucleotides targeted to the Hepatitis B genome;
   for use in combination to treat or prevent a viral infection, such as Hepatitis D.

In certain embodiments, the kit comprises at least three oligomeric nucleotides targeted to the Hepatitis B genome. In certain embodiments, the kit comprises oligomeric nucleotides 3m, 6m and 12m as described herein. In certain embodiments, the oligomeric nucleotides are comprised within a lipid nanoparticle formulation.

In certain embodiments, the kit comprises one of the following combinations of two agents:

the RNA destabilizer and the capsid inhibitor;
at least one oligomeric nucleotide targeted to the Hepatitis B genome and the capsid inhibitor;
at least one oligomeric nucleotide targeted to the Hepatitis B genome and the RNA destabilizer;
at least one oligomeric nucleotide targeted to the Hepatitis B genome and a reverse transcriptase inhibitor;
the capsid inhibitor and a reverse transcriptase inhibitor; or
the RNA destabilizer and a reverse transcriptase inhibitor.

In certain embodiments, the kit comprises one of the following combinations of two agents:

the RNA destabilizer and the capsid inhibitor;
a combination of oligomeric nucleotides 3m, 6m and 12m and the capsid inhibitor;
the capsid inhibitor and tenofovir disoproxil fumarate;
the capsid inhibitor and tenofovir alafenamide;
the capsid inhibitor and entecavir;
the RNA destabilizer and tenofovir disoproxil fumarate;
the RNA destabilizer and tenofovir alafenamide; or
the RNA destabilizer and entecavir.

In certain embodiments, the kit comprises the RNA destabilizer (compound 2) and the capsid inhibitor (compound 1).

In certain embodiments, the kit comprises a combination of oligomeric nucleotides 3m, 6m and 12m and the capsid inhibitor (compound 1).

In certain embodiments, the kit comprises the capsid inhibitor (compound 1) and tenofovir disoproxil fumarate.

In certain embodiments, the kit comprises the capsid inhibitor (compound 1) and tenofovir alafenamide.

In certain embodiments, the kit comprises the capsid inhibitor (compound 1) and entecavir.

In certain embodiments, the kit comprises the RNA destabilizer (compound 2) and tenofovir disoproxil fumarate.

In certain embodiments, the kit comprises the RNA destabilizer (compound 2) and tenofovir alafenamide.

In certain embodiments, the kit comprises the RNA destabilizer (compound 2) and entecavir.

In certain embodiments, the kit comprises one of the following combinations of three agents:

the capsid inhibitor, the RNA destabilizer and a reverse transcriptase inhibitor;
the capsid inhibitor, at least one oligomeric nucleotide targeted to the Hepatitis B genome and a reverse transcriptase inhibitor;
the capsid inhibitor, the RNA destabilizer and at least one oligomeric nucleotide targeted to the Hepatitis B genome; or
the RNA destabilizer, at least one oligomeric nucleotide targeted to the Hepatitis B genome and a reverse transcriptase inhibitor.

In certain embodiments, the kit comprises one of the following combinations of three agents:

the capsid inhibitor, the RNA destabilizer and tenofovir disoproxil fumarate;
the capsid inhibitor, the RNA destabilizer and tenofovir alafenamide; or
the capsid inhibitor, the RNA destabilizer and entecavir.

In certain embodiments, the kit comprises the capsid inhibitor (compound 1), the RNA destabilizer (compound 2) and tenofovir disoproxil fumarate.

In certain embodiments, the kit comprises the capsid inhibitor (compound 1), the RNA destabilizer (compound 2) and tenofovir alafenamide; or

In certain embodiments, the kit comprises the capsid inhibitor (compound 1), the RNA destabilizer (compound 2) and entecavir.

Certain embodiments of the invention provide a method for treating hepatitis B in an animal comprising administering to the animal, at least two agents selected from the group consisting of:
a) a capsid inhibitor, wherein the capsid inhibitor is:

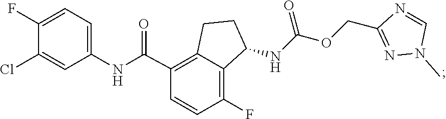 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

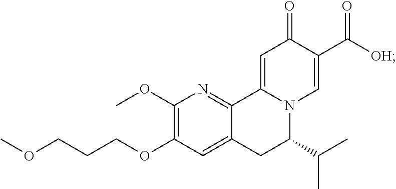 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and
d) oligomeric nucleotides targeted to the Hepatitis B genome.

Certain embodiments of the invention provide a method for treating hepatitis B in an animal comprising administering to the animal, at least two agents selected from the group consisting of:
a) a capsid inhibitor, wherein the capsid inhibitor is:

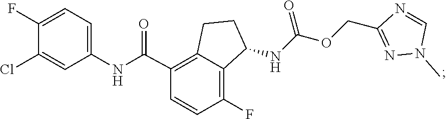 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

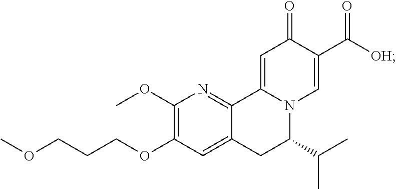 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and
d) oligomeric nucleotides targeted to the Hepatitis B genome,
provided at least one of the agents administering to the animal is the capsid inhibitor or the RNA destabilizer.

Certain embodiments of the invention provide a method for treating hepatitis B in an animal comprising administering to the animal, at least two agents selected from the group consisting of:

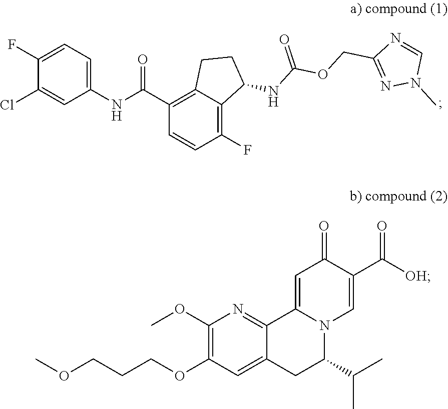

c) a compound selected from the group consisting of tenofovir disoproxil fumarate, tenofovir alafenamide and entecavir; and
d) oligomeric nucleotides targeted to the Hepatitis B genome.

Certain embodiments of the invention provide a method for treating hepatitis D in an animal comprising administering to the animal, at least two agents selected from the group consisting of:
a) a capsid inhibitor, wherein the capsid inhibitor is:

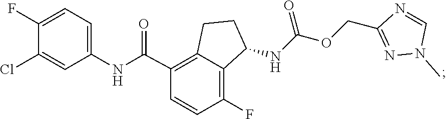 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

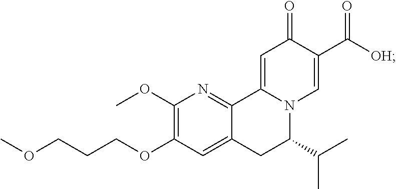 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and
d) oligomeric nucleotides targeted to the Hepatitis B genome.

In certain embodiments, at least three oligomeric nucleotides targeted to the Hepatitis B genome are administered to the animal. In certain embodiments, oligomeric nucleotides 3m, 6m and 12m as described herein are administered to the animal. In certain embodiments, the oligomeric nucleotides are comprised within a lipid nanoparticle formulation.

In certain embodiments at least one agent is administered orally. In certain embodiments at least two agents are administered orally. In certain embodiments at least one oligomeric nucleotide is administered intravenously.

In certain embodiments, one of the following combinations of two agents is administered to the animal:

the RNA destabilizer and the capsid inhibitor;
at least one oligomeric nucleotide targeted to the Hepatitis B genome and the capsid inhibitor;
at least one oligomeric nucleotide targeted to the Hepatitis B genome and the RNA destabilizer;
at least one oligomeric nucleotide targeted to the Hepatitis B genome and a reverse transcriptase inhibitor;
the capsid inhibitor and a reverse transcriptase inhibitor; or
the RNA destabilizer and a reverse transcriptase inhibitor.

In certain embodiments, one of the following combinations of two agents is administered to the animal:

the RNA destabilizer and the capsid inhibitor;
a combination of oligomeric nucleotides 3m, 6m and 12m and the capsid inhibitor;
the capsid inhibitor and tenofovir disoproxil fumarate;
the capsid inhibitor and tenofovir alafenamide;
the capsid inhibitor and entecavir;
the RNA destabilizer and tenofovir disoproxil fumarate;
the RNA destabilizer and tenofovir alafenamide; or
the RNA destabilizer and entecavir.

In certain embodiments, the RNA destabilizer (compound 2) and the capsid inhibitor (compound 1) are administered to the animal.

In certain embodiments, a combination of oligomeric nucleotides 3m, 6m and 12m and the capsid inhibitor (compound 1) are administered to the animal.

In certain embodiments, the capsid inhibitor (compound 1) and tenofovir disoproxil fumarate are administered to the animal.

In certain embodiments, the capsid inhibitor (compound 1) and tenofovir alafenamide are administered to the animal.

In certain embodiments, the capsid inhibitor (compound 1) and entecavir are administered to the animal.

In certain embodiments, the RNA destabilizer (compound 2) and tenofovir disoproxil fumarate are administered to the animal.

In certain embodiments, the RNA destabilizer (compound 2) and tenofovir alafenamide are administered to the animal.

In certain embodiments, the RNA destabilizer (compound 2) and entecavir are administered to the animal.

In certain embodiments, one of the following combinations of three agents is administered to the animal:

the capsid inhibitor, the RNA destabilizer and a reverse transcriptase inhibitor;
the capsid inhibitor, at least one oligomeric nucleotide targeted to the Hepatitis B genome and a reverse transcriptase inhibitor;
the capsid inhibitor, the RNA destabilizer and at least one oligomeric nucleotide targeted to the Hepatitis B genome; or
the RNA destabilizer, at least one oligomeric nucleotide targeted to the Hepatitis B genome and a reverse transcriptase inhibitor.

In certain embodiments, one of the following combinations of three agents is administered to the animal:

the capsid inhibitor, the RNA destabilizer and tenofovir disoproxil fumarate;
the capsid inhibitor, the RNA destabilizer and tenofovir alafenamide; or
the capsid inhibitor, the RNA destabilizer and entecavir.

In certain embodiments, the capsid inhibitor (compound 1), the RNA destabilizer (compound 2) and tenofovir disoproxil fumarate are administered to the animal.

In certain embodiments, the capsid inhibitor (compound 1), the RNA destabilizer (compound 2) and tenofovir alafenamide are administered to the animal.

In certain embodiments, the capsid inhibitor (compound 1), the RNA destabilizer (compound 2) and entecavir are administered to the animal.

Certain embodiments also provide a combination of at least two agents selected from the group consisting of:

a) a capsid inhibitor, wherein the capsid inhibitor is:

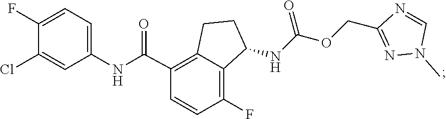 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

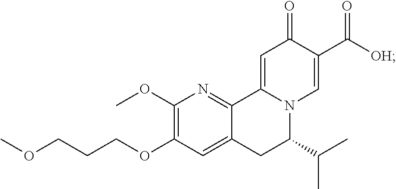 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and

d) oligomeric nucleotides targeted to the Hepatitis B genome, for use in treating Hepatitis B or Hepatitis D in an animal.

Certain embodiments also provide the use of a combination of at least two agents selected from the group consisting of:

a) a capsid inhibitor, wherein the capsid inhibitor is:

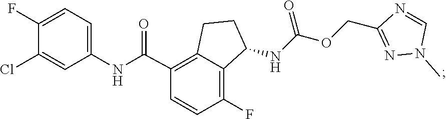 [SMILES: Cn1cnc(COC(=O)N[C@H]2CCc3c(C(=O)Nc4ccc(F)c(Cl)c4)ccc(F)c32)n1]

b) an RNA destabilizer, wherein the RNA destabilizer is:

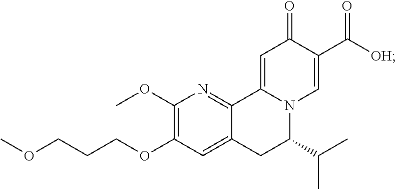 [SMILES: COCCCOc1cc2c(nc1OC)-c1cc(=O)c(C(=O)O)cn1[C@H](C(C)C)C2]

c) reverse transcriptase inhibitors selected from the group consisting of tenofovir disoproxil fumarate, tenofovir alafenamide and entecavir; and

d) oligomeric nucleotides targeted to the Hepatitis B genome, in the manufacture of a medicament for the treatment of Hepatitis B or Hepatitis D in an animal.

In certain embodiments, the combination is a combination described herein.

The ability of a combination of therapeutic agents to treat Hepatitis B may be determined using pharmacological models which are well known to the art. The ability of a combination of therapeutic agents to treat Hepatitis D may be determined using pharmacological models which are well known to the art.

The invention will now be illustrated by the following non-limiting Examples. It should be understood that the numbering of compounds and Tables within the described sets of Examples may be specific to those sets of Examples.

### Examples 1-4

The materials and methods for combination studies in primary human hepatocytes (PHHs) are described below in Examples 1-4.

### PHHs

Cryopreserved PHHs (Lot IKB) were purchased from BioreclamationIVT

### Test Articles

Compounds (V), (VI) and (VII) were produced by Arbutus Biopharma. Pegylated IFN-α2a and TAF were purchased commercially. Information on the compounds is shown in Table 1.

TABLE 1
Information on test articles

| Compound | Vial ID | M.W. | Weight |
|---|---|---|---|
| V | 161534616 | | 30 mg total |
| | | | amount in |
| | | | solution |
| VII | 1ACGJ | 461.85 | 10.2 mg |
| VI | 1A45C | 402.45 | 17.7 mg |

| Compound | Vendor | Catalog No. | Stock Conc. |
|---|---|---|---|
| PEG-IFNα2a | Roche | B3044-SH0337 | 180 μg/0.5 ml |
| | | | (5040000 IU/ml) |
| TAF | Selleck | S7856-01 | 20 mM |

### Infectious Virus Stock

D type HBV was concentrated from HepG2DE19 culture supernatants. Information on the viruses is shown in Table 2.

TABLE 2
Information on HBV virus stock

| | | HBV titer | | |
|---|---|---|---|---|
| | | in serum | HBV | |
| Virus ID | Lot# | (GE*/ml) | Genotype | Source |
| HBV-DE19 | 20180313 | 3.2E+10 GE/ml | D | HepDE19 |
| | | | | supernatants |

*GE = HBV genome equivalent.

### Reagents

The major reagents used in the study were QIAamp 96 DNA Blood Kit (QIAGEN #51162), FastStart Universal Probe Master (Roche #04914058001), CellTiter-Glo (Promega #G7573) and HBsAg ELISA kit (Antu #CL 0310), and Lipofectamine 3000 (ThermoFisher #L3000015).

### Instruments

The major instruments used in the study were BioTek Synergy 2, SpectraMax (Molecular Devices), and 7900HT Fast Real-Time PCR System (ABI).

### Study Procedure

### Seeding of Primary Human Hepatocytes

The PHH were thawed and seeded into 48-well plates at a density of 1.32×10⁵ cells/well. The day PHH seeding date was defined as day 0.

### HBV Infection

The PHH were infected with 400 HBV GE/cell of HBV genotype D type HBV on day 1.

### Culture and Treatment of PHHs.

On day 0, 6-8 hours after cell seeding, the compound of formula (V) was serially diluted with media containing the transfection reagent to make 26.55× (for single compound dose response study) or 265.5× (for double combination studies) of the final test concentrations. The test articles were further diluted with the culture medium to the final test concentrations.

On day 2, the compounds of formula (VI) and (VII), and TAF were serially diluted with DMSO to make 100× of the final test concentrations. PEG-IFNα2a was serially diluted in culture medium to make 100× of the final test concentrations. All the test articles were further diluted 100 times with the culture medium. The final concentration of DMSO in the culture medium was 2%.

### Determination of EC₅₀ Values.

The compounds were tested at 7 concentrations, 3-fold dilution, in triplicate.

### Double Combination Study.

Four two-way combinations were performed on a 5×5 matrix, in triplicate plates. Transfection reagent was present in all wells. The culture medium containing the articles were refreshed every 1 or 2 days.

### Assay for Cytotoxicity by CellTiter Glo Assay at Day 8

One day 8, the culture supernatants were collected, and CellTiter-Glo working solution was added to the cell plates. The plates were incubated at room temperature 10 mins. The lysates were transferred into a 96-well black plate. Luminescence signal was measured on a BioTek Synergy 2 SpectraMax. Percent cell viability was calculated with the formula below:

Viability %=(raw data of sample−AVG. of blank)/(AVG. of Medium control−AVG. of blank)×100

Quantification of HBV DNA in the Culture Supernatants by qPCR

DNA in the culture supernatants harvested on days 8 was isolated with QIAamp 96 DNA Blood Kit (Qiagen-51162). For each sample, 100 μl of each culture supernatant was used to extract DNA. The DNA was eluted with 180 μl of AE. HBV DNA in the culture supernatants was quantified by quatitative PCR using primers and probes outlined in Table 3. Percent inhibition of HBV DNA was calculated with the formula below:

% Inh. HBV DNA=[1−value of sample/AVG. value of Medium control]×100.

TABLE 3
Primer/Probe information

| | |
|---|---|
| Primer R | GACAAACGGGCAACATACCTT |
| Primer F | GTGTCTGCGGCGTTTTATCA |
| Probe | 5'FAM CCTCTKCATCCTGCTGCTATGCCTCATC 3’TAMRA |

### Measurement of HBsAg in the Culture Supernatants by ELISA

HBsAg in the culture supernatants harvested on days 8 was measured using the HBsAg/ELISA kit (Autobio) according to the manual. The samples were diluted with PBS to get the signal in the range of the standard curve. Percent inhibition of HBsAg was calculated with the formula below:

% Inh. HBsAg=[1−HBsAg quantity of sample/HBV quantity of DMSO control]×100

### Analysis of Combination Effects

Results of double combination studies were analyzed using MacSynergy II software (Prichard and Shipman, 1992). Combination effects were calculated as synergy/antagonism volumes to 99.9% confidence interval, and results were interpreted according to MacSynergy II guidelines, as follows:

<25=Insignificant synergism/antagonism

25-50=Minor but significant synergism/antagonism

50-100=Moderate synergism/antagonism

>100=Strong synergism/antagonism

˜1000=Possible errors

### Compound of Formula (V)

The compound of formula (V) is an siRNA agent that acts on all HBV RNA transcripts, enabling inhibition of HBV replication and suppression of all viral antigens including HBsAg. A high avidity N-acetylgalactosamine (GalNAc) moiety mediates targeting of the compound to hepatocytes, the site of HBV infection. The compound of formula (V) is described in International Publication Number WO2018/191278 (International application number PCT/US2018/026918), which published on Oct. 18, 2018).

In certain embodiments, the GalNAc Moeity has the following structure:

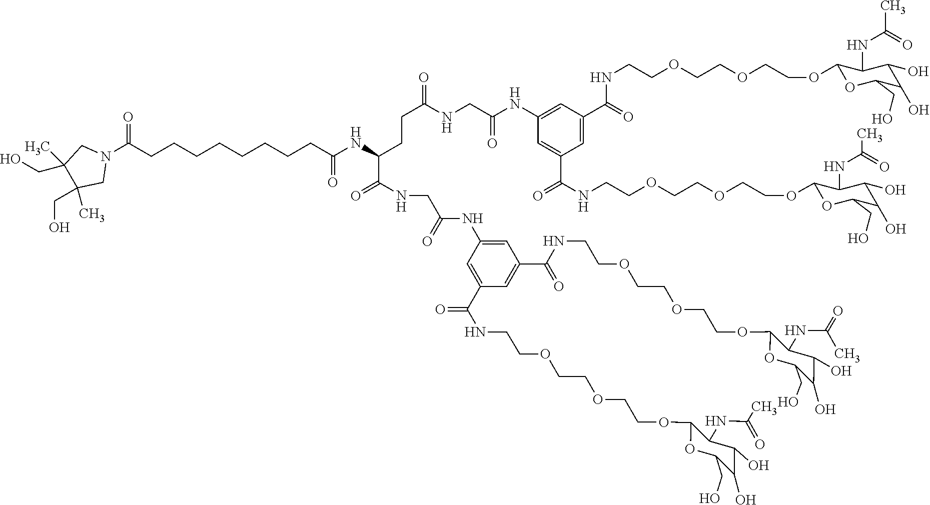 [SMILES: CC(=O)NC1C(OCCOCCOCCNC(=O)c2cc(NC(=O)CNC(=O)CC[C@H](NC(=O)CCCCCCCCC(=O)N3CC(C)(CO)C(C)(CO)C3)C(=O)NCC(=O)Nc3cc(C(=O)NCCOCCOCCOC4OC(CO)C(O)C(O)C4NC(C)=O)cc(C(=O)NCCOCCOCCOC4OC(CO)C(O)C(O)C4NC(C)=O)c3)cc(C(=O)NCCOCCOCCOC3OC(CO)C(O)C(O)C3NC(C)=O)c2)OC(CO)C(O)C1O]

In certain embodiments, the siRNA of the siRNA conjugate is siRNA 1. In certain embodiments, the siRNA of the siRNA conjugate is siRNA 2. In the experiments described hereinbelow, the siRNA of the siRNA conjugate is siRNA 2. The compound of formula (V) is depicted below, wherein the siRNA of the siRNA conjugate is siRNA 2.

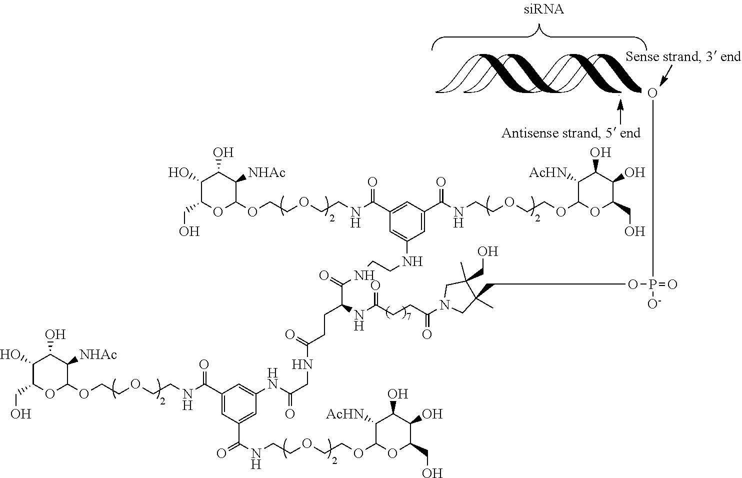

| siRNA Name | Sense Sequence (5′-3′) | Antisense Sequence (5′-3′) |
|---|---|---|
| siRNA 1 | usgscaCUUcgcuucaccu | asGsgugaagcgaagUgCacascsgU |
| siRNA 2 | gsusgcACUucgcuucaca | usGsugaagcgaaguGcAcacsgsgU |

2’-O-Methyl nucleotides = lower case
2’-Fluoro nucleotides = UPPER_CASE
Phosphorothioate linker = s
Unmodified = UPPER CASE

### Pegylated Interferon Alpha 2a (IFNα2a):

This agent was purchased from a commercial source:

| | | | Size/Stock |
|---|---|---|---|
| Compound | Vendor | Catalog No. | Concentration |
| PEG-IFNα2a | Roche | B3044-SH0337 | 180 μg/0.5 ml |
| | | | (5040000 IU/ml) |

### Small Molecule Compounds

TABLE 4
Structures

| Compound | Structure |
|---|---|
| VII | 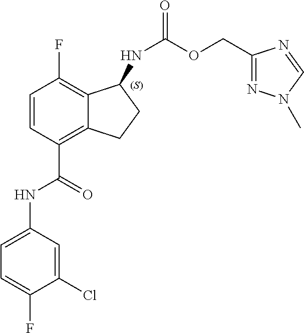 |
| VI | 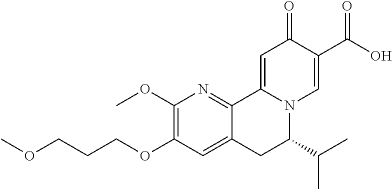 [SMILES: COCCCOc1cc2c(nc1OC)-c1cc(=O)c(C(=O)O)cn1[C@H](C(C)C)C2] |
| Tenofovir Alafenamide Fumarate (TAF) | 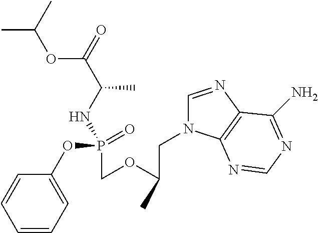 [SMILES: CC(C)OC(=O)[C@H](C)N[P@](=O)(CO[C@H](C)Cn1cnc2c(N)ncnc21)Oc1ccccc1] |

### Information on the Following Small Molecule Compounds:

| Compound | Vial ID | Molecular Weight | Weight |
|---|---|---|---|
| VII | 1ACGJ | 461.85 | 10.2 mg |
| VI | 1A45C | 402.45 | 17.7 mg |

### Information on Commercially Available TAF:

| | | Catalog | Stock |
|---|---|---|---|
| Compound | Vendor | Number | Concentration |
| TAF | SelleckChem | S7856-01 | 20 mM |

### Example 1. In Vitro Combination of Compounds of Formula (V) and (VI)

### Study Goal

To determine whether a two-drug combination of a compound of formula (V) (a GalNAc-conjugated siRNA targeting the HBV genome, and inhibiting production of HBV DNA, HBsAg and HBeAg, and HBx), and a compound of formula (VI) (a small molecule inhibitor of HBV RNA stability that inhibits HBV DNA, HBsAg and HBeAg) is additive, synergistic or antagonistic in vitro, using HBV-infected human primary hepatocytes in a cell culture model system.

### Results and Conclusion

The compound of formula (VI) (concentration range of 4.00 μM to 0.05 μM in a 3-fold dilution series and 5-point titration) was tested in combination with a compound of formula (V) (concentration range of 3.0 μg/mL to 0.04 μg/mL in a 3-fold dilution series and 5-point titration), on three replicate plates in each of two separate experimental trials. The average % inhibition in HBV DNA and HBsAg, and standard deviations of 3 replicates observed either with a compound of formula (V) or a compound of formula (VI) treatments alone or in combination are shown in Tables 5A, 5B, 6A, and 6B as indicated below. The EC₅₀ values of a compound of formula (V) and a compound of formula (VI) were determined in an earlier experiment and are shown in Table 7.

When the observed values of a two-inhibitor combination were compared to what is expected from additive interaction for the above concentration range, the combination effects ranged from additive for HBsAg inhibition, with no significant synergy or antagonism, to additive to minor synergy for HBV DNA inhibition, as per MacSynergy II analysis at 99.9% confidence interval, and using the interpretive criteria described by Prichard and Shipman (1992) (Table 7). No significant inhibition of cell viability was observed by microscopy or CellTiter-Glo assay.

TABLE 5A
Experiment 1: Effect on HBV DNA in In Vitro Combination
of Compounds of Formula (V) and (VI)

| [DRUG] | | | | | | | AVERAGE % INHIBITION |
|---|---|---|---|---|---|---|---|
| (VI) | | | | | | | Horizontal axis: |
| μM | 0.00 | 0.04 | 0.11 | 0.33 | 1.00 | 3.00 | (V) (μg/mL) |
| 0.400 | 21.77 | 47.71 | 47.75 | 45.41 | 49.05 | 53.96 | |
| 0.133 | 26.74 | 54.09 | 46.95 | 57.95 | 58.35 | 61.81 | |
| 0.044 | 10.93 | 51.98 | 58.48 | 57.38 | 59.64 | 65.82 | |
| 0.015 | 6.77 | 43.37 | 48.79 | 41.96 | 55.01 | 52.55 | |
| 0.005 | 0.64 | 33.1 | 43.41 | 55.54 | 48.43 | 57.43 | |
| 0.00 | 0 | 26.41 | 32.08 | 25.93 | 39.52 | 42.88 | |

| [DRUG] | | | | | | | STANDARD DEVIATION (%) |
|---|---|---|---|---|---|---|---|
| (VI) | | | | | | | Horizontal axis: |
| μM | 0.00 | 0.04 | 0.11 | 0.33 | 1.00 | 3.00 | (V) (μg/mL) |
| 0.400 | 4.91 | 5.57 | 7.14 | 10.51 | 6.04 | 5.02 | |
| 0.133 | 3.6 | 4.43 | 9.97 | 3.74 | 4.41 | 7.46 | |
| 0.044 | 20.26 | 0.75 | 1.38 | 6.32 | 5.44 | 2.29 | |
| 0.015 | 16.57 | 7.86 | 12.69 | 9.74 | 7.94 | 12.18 | |
| 0.005 | 16.7 | 24.4 | 17.87 | 8.37 | 2 | 4.23 | |
| 0.00 | 16.92 | 9.01 | 11.38 | 9.45 | 0.65 | 6.33 | |

| [DRUG] | | | | | | | ADDITIVE % INHIBITION |
|---|---|---|---|---|---|---|---|
| (VI) | | | | | | | Horizontal axis: |
| μM | 0.00 | 0.04 | 0.11 | 0.33 | 1.00 | 3.00 | (V) (μg/mL) |
| 0.400 | 21.77 | 42.43 | 46.87 | 42.06 | 52.69 | 55.32 | |
| 0.133 | 26.74 | 46.09 | 50.24 | 45.74 | 55.69 | 58.15 | |
| 0.044 | 10.93 | 34.45 | 39.5 | 34.03 | 46.13 | 49.12 | |
| 0.015 | 6.77 | 31.39 | 36.68 | 30.94 | 43.61 | 46.75 | |
| 0.005 | 0.64 | 26.88 | 32.51 | 26.4 | 39.91 | 43.25 | |
| 0.00 | 0 | 26.41 | 32.08 | 25.93 | 39.52 | 42.88 | |

| [DRUG] | | | | | | | SYNERGY PLOT (99.9%) |
|---|---|---|---|---|---|---|---|
| (VI) | | | | | | | Bonferroni Adj. |
| μM | 0.00 | 0.04 | 0.11 | 0.33 | 1.00 | 3.00 | 98% |
| 0.400 | 0 | 0 | 0 | 0 | 0 | 0 | SYNERGY 45.01 |
| 0.133 | 0 | 0 | 0 | 0 | 0 | 0 | log volume 10.25 |
| 0.044 | 0 | 15.06175 | 14.43842 | 2.55088 | 0 | 9.16361 | |
| 0.015 | 0 | 0 | 0 | 0 | 0 | 0 | ANTAGONISM 0 |
| 0.005 | 0 | 0 | 0 | 1.59433 | 1.938 | 0.25907 | log volume 0 |
| 0.00 | 0 | 0 | 0 | 0 | 0 | 0 | |

TABLE 5B
Experiment 2: Effect on HBV DNA in In Vitro Combination
of Compounds of Formula (V) and (VI)

| [DRUG] | | | | | | | AVERAGE % INHIBITION |
|---|---|---|---|---|---|---|---|
| (VI) | | | | | | | Horizontal axis: |
| μM | 0.00 | 0.04 | 0.11 | 0.33 | 1.00 | 3.00 | (V) (μg/mL) |
| 0.400 | 27.15 | 39.97 | 31.05 | 27.97 | 34.71 | 44.56 | |
| 0.133 | 12.75 | 42.49 | 46.25 | 40.38 | 39.39 | 44.6 | |
| 0.044 | 19.27 | 47.13 | 44.38 | 49.83 | 46.17 | 44.54 | |
| 0.015 | 13.74 | 38.7 | 40.44 | 48.58 | 41.66 | 40.01 | |
| 0.005 | 7.21 | 27.49 | 43.57 | 42.57 | 34.31 | 30.66 | |
| 0.00 | 0 | 19.52 | 31.64 | 28.12 | 21.67 | 18.23 | |

| [DRUG] | | | | | | | STANDARD DEVIATION (%) |
|---|---|---|---|---|---|---|---|
| (VI) | | | | | | | Horizontal axis: |
| μM | 0.00 | 0.04 | 0.11 | 0.33 | 1.00 | 3.00 | (V) (μg/mL) |
| 0.400 | 13.35 | 9.43 | 20.97 | 19.17 | 14.01 | 5.11 | |
| 0.133 | 9.76 | 11.87 | 5.61 | 9.05 | 4.85 | 7.79 | |
| 0.044 | 19.61 | 2.84 | 5.1 | 11.92 | 5.37 | 5.22 | |
| 0.015 | 10.82 | 5.69 | 1.11 | 2.67 | 8.21 | 5.4 | |
| 0.005 | 17.73 | 15.32 | 5.36 | 14.84 | 11.93 | 10.94 | |
| 0.00 | 22.91 | 8.74 | 11.47 | 2.17 | 17.71 | 21.6 | |

| [DRUG] | | | | | | | ADDITIVE % INHIBITION |
|---|---|---|---|---|---|---|---|
| (VI) | | | | | | | Horizontal axis: |
| μM | 0.00 | 0.04 | 0.11 | 0.33 | 1.00 | 3.00 | (V) (μg/mL) |
| 0.400 | 27.15 | 41.37 | 50.2 | 47.64 | 42.94 | 40.43 | |
| 0.133 | 12.75 | 29.78 | 40.36 | 37.28 | 31.66 | 28.66 | |
| 0.044 | 19.27 | 35.03 | 44.81 | 41.97 | 36.76 | 33.99 | |
| 0.015 | 13.74 | 30.58 | 41.03 | 38 | 32.43 | 29.47 | |
| 0.005 | 7.21 | 25.32 | 36.57 | 33.3 | 27.32 | 24.13 | |
| 0.00 | 0 | 19.52 | 31.64 | 28.12 | 21.67 | 18.23 | |

| [DRUG] | | | | | | | SYNERGY PLOT (99.9%) |
|---|---|---|---|---|---|---|---|
| (VI) | | | | | | | Bonferroni Adj. |
| μM | 0.00 | 0.04 | 0.11 | 0.33 | 1.00 | 3.00 | 98% |
| 0.400 | 0 | 0 | 0 | 0 | 0 | 0 | SYNERGY 4.55 |
| 0.133 | 0 | 0 | 0 | 0 | 0 | 0 | log volume 1.04 |
| 0.044 | 0 | 2.75356 | 0 | 0 | 0 | 0 | |
| 0.015 | 0 | 0 | 0 | 1.79303 | 0 | 0 | ANTAGONISM 0 |
| 0.005 | 0 | 0 | 0 | 0 | 0 | 0 | log volume 0 |
| 0.00 | 0 | 0 | 0 | 0 | 0 | 0 | |

TABLE 6A
Experiment 1: Effect on HBsAg in In Vitro Combination of Compounds of Formula (V) and VI

| [DRUG] | | | | | | | AVERAGE % INHIBITION |
|---|---|---|---|---|---|---|---|
| (VI) | | | | | | | Horizontal axis: |
| μM | 0.00 | 0.04 | 0.11 | 0.33 | 1.00 | 3.00 | (V) (μg/mL) |
| 0.400 | 80.95 | 87.46 | 89.86 | 92.04 | 93.65 | 95.03 | |
| 0.133 | 74.05 | 84.37 | 88.51 | 91.2 | 93.12 | 94.44 | |
| 0.044 | 62.19 | 78.25 | 85.08 | 88.89 | 91.46 | 94 | |
| 0.015 | 43.07 | 70.63 | 78.96 | 85.26 | 89.62 | 92.59 | |
| 0.005 | 19.15 | 61.93 | 74.76 | 82.34 | 87.79 | 91.46 | |
| 0.00 | 0 | 57.32 | 69.46 | 80.84 | 86.11 | 90.05 | |

| [DRUG] | | | | | | | STANDARD DEVIATION (%) |
|---|---|---|---|---|---|---|---|
| (VI) | | | | | | | Horizontal axis: |
| μM | 0.00 | 0.04 | 0.11 | 0.33 | 1.00 | 3.00 | (V) (μg/mL) |
| 0.400 | 2.05 | 0.86 | 1 | 1.06 | 0.9 | 0.98 | |
| 0.133 | 0.86 | 1.09 | 1.19 | 0.15 | 0.56 | 0.75 | |
| 0.044 | 1.55 | 0.78 | 1.08 | 0.23 | 0.3 | 0.52 | |
| 0.015 | 1.35 | 2.06 | 0.67 | 1.12 | 0.55 | 1.08 | |
| 0.005 | 2.33 | 0.74 | 1.18 | 0.79 | 0.41 | 1.06 | |
| 0.00 | 3.32 | 1.13 | 1.83 | 1.03 | 0.53 | 1.4 | |

| [DRUG] | | | | | | | ADDITIVE % INHIBITION |
|---|---|---|---|---|---|---|---|
| (VI) | | | | | | | Horizontal axis: |
| μM | 0.00 | 0.04 | 0.11 | 0.33 | 1.00 | 3.00 | (V) (μg/mL) |
| 0.400 | 80.95 | 91.87 | 94.18 | 96.35 | 97.35 | 98.1 | |
| 0.133 | 74.05 | 88.92 | 92.07 | 95.03 | 96.4 | 97.42 | |
| 0.044 | 62.19 | 83.86 | 88.45 | 92.76 | 94.75 | 96.24 | |
| 0.015 | 43.07 | 75.7 | 82.61 | 89.09 | 92.09 | 94.34 | |
| 0.005 | 19.15 | 65.49 | 75.31 | 84.51 | 88.77 | 91.96 | |
| 0.00 | 0 | 57.32 | 69.46 | 80.84 | 86.11 | 90.05 | |

| [DRUG] | | | | | | | SYNERGY PLOT (99.9%) |
|---|---|---|---|---|---|---|---|
| (VI) | | | | | | | Bonferroni Adj. |
| μM | 0.00 | 0.04 | 0.11 | 0.33 | 1.00 | 3.00 | 98% |
| 0.400 | 0 | −1.57974 | −1.029 | −0.82154 | −0.7381 | 0 | SYNERGY 0 |
| 0.133 | 0 | −0.96281 | 0 | −3.33635 | −1.43704 | −0.51175 | log volume 0 |
| 0.044 | 0 | −3.04302 | 0 | −3.11307 | −2.3027 | −0.52868 | |
| 0.015 | 0 | 0 | −1.44503 | −0.14408 | −0.65995 | 0 | ANTAGONISM −22.78 |
| 0.005 | 0 | −1.12466 | 0 | 0 | 0 | 0 | log volume −5.19 |
| 0.00 | 0 | 0 | 0 | 0 | 0 | 0 | |

TABLE 6B
Experiment 2: Effect on HBsAg in In Vitro Combination of Compounds of Formula (V) and (VI)

| [DRUG] | | | | | | | AVERAGE % INHIBITION |
|---|---|---|---|---|---|---|---|
| (VI) | | | | | | | Horizontal axis: |
| μM | 0.00 | 0.04 | 0.11 | 0.33 | 1.00 | 3.00 | (V) (μg/mL) |
| 0.400 | 77.44 | 85.56 | 87.9 | 91.58 | 93.52 | 95.28 | |
| 0.133 | 69.71 | 83.49 | 86.42 | 90.62 | 92.52 | 94.49 | |
| 0.044 | 56.72 | 77.81 | 84.18 | 89.61 | 91.58 | 93.99 | |
| 0.015 | 38.14 | 70.78 | 81.22 | 87.1 | 90.26 | 92.72 | |
| 0.005 | 11.89 | 59.92 | 76.39 | 84 | 87.94 | 91.62 | |
| 0.00 | 0 | 62.55 | 76.45 | 84.3 | 89.08 | 92.24 | |

| [DRUG] | | | | | | | STANDARD DEVIATION (%) |
|---|---|---|---|---|---|---|---|
| (VI) | | | | | | | Horizontal axis: |
| μM | 0.00 | 0.04 | 0.11 | 0.33 | 1.00 | 3.00 | (V) (μg/mL) |
| 0.400 | 2.18 | 1.64 | 1.79 | 0.92 | 0.86 | 0.22 | |
| 0.133 | 2 | 1.5 | 1.72 | 1.05 | 0.53 | 0.31 | |
| 0.044 | 2.42 | 3.42 | 2.09 | 1.5 | 0.89 | 0.58 | |
| 0.015 | 2.27 | 4.01 | 2.04 | 1.66 | 1.11 | 0.6 | |
| 0.005 | 5.15 | 2.63 | 3.15 | 2.03 | 1.56 | 0.28 | |
| 0.00 | 10.73 | 0.82 | 0.85 | 0.99 | 1.04 | 0.73 | |

| [DRUG] | | | | | | | ADDITIVE % INHIBITION |
|---|---|---|---|---|---|---|---|
| (VI) | | | | | | | Horizontal axis: |
| μM | 0.00 | 0.04 | 0.11 | 0.33 | 1.00 | 3.00 | (V) (μg/mL) |
| 0.400 | 77.44 | 91.55 | 94.69 | 96.46 | 97.54 | 98.25 | |
| 0.133 | 69.71 | 88.66 | 92.87 | 95.24 | 96.69 | 97.65 | |
| 0.044 | 56.72 | 83.79 | 89.81 | 93.21 | 95.27 | 96.64 | |
| 0.015 | 38.14 | 76.83 | 85.43 | 90.29 | 93.24 | 95.2 | |
| 0.005 | 11.89 | 67 | 79.25 | 86.17 | 90.38 | 93.16 | |
| 0.00 | 0 | 62.55 | 76.45 | 84.3 | 89.08 | 92.24 | |

| [DRUG] | | | | | | | SYNERGY PLOT (99.9%) |
|---|---|---|---|---|---|---|---|
| (VI) | | | | | | | Bonferroni Adj. |
| μM | 0.00 | 0.04 | 0.11 | 0.33 | 1.00 | 3.00 | 98% |
| 0.400 | 0 | −0.59276 | −0.89911 | −1.85228 | −1.18974 | −2.24598 | SYNERGY 0 |
| 0.133 | 0 | −0.2335 | −0.78948 | −1.16445 | −2.42577 | −2.13979 | log volume 0 |
| 0.044 | 0 | 0 | 0 | 0 | −0.76101 | −0.74122 | |
| 0.015 | 0 | 0 | 0 | 0 | 0 | −0.5054 | ANTAGONISM −16.16 |
| 0.005 | 0 | 0 | 0 | 0 | 0 | −0.61852 | log volume −3.68 |
| 0.00 | 0 | 0 | 0 | 0 | 0 | 0 | |

TABLE 7
Summary of results of in vitro combination studies of compounds
of formula (V) and (VI) in PHH cell culture system:

| HBV | (VI) | (V) | | Synergy | | Antagonism | |
|---|---|---|---|---|---|---|---|
| Assay | EC₅₀ | EC₅₀ | Synergy | Log | Antagonism | Log | |
| Endpoint | (μM)# | (μg/mL)# | Volume* | Volume* | Volume* | Volume* | Conclusion |
| HBV | 0.006 | <0.123 | 45.01, 4.55 | 10.25, 1.04 | 0, 0 | 0, 0 | Additive to |
| DNA | | | | | | | Minor Synergy |
| HBsAg | 0.013 | <0.123 | 0, 0 | 0, 0 | −22.78, −16.16 | −5.19, −3.68 | Additive |

*at 99.9% confidence interval
#determined in an earlier separate experiment

### Example 2. In Vitro Combination of Compounds of Formula (V) and (VII)

### Study Goal

To determine whether a two-drug combination of a compound of formula (V) (a GalNAc-conjugated siRNA targeting the HBV genome, and inhibiting production of HBV DNA, HBsAg and HBeAg, and HBx), and a compound of formula (VII) (a small molecule inhibitor of HBV capsid assembly) is additive, synergistic or antagonistic in vitro, using HBV-infected human primary hepatocytes in a cell culture model system.

### Results and Conclusion

A compound of formula (VII) (concentration range of 4.00 μM to 0.05 μM in a 3-fold dilution series and 5-point titration) was tested in combination with a compound of formula (V) (concentration range of 3.0 μg/mL to 0.04 μg/mL in a 3-fold dilution series and 5-point titration), on three replicate plates in each of two separate experimental trials. The average % inhibition in HBV DNA and HBsAg, and standard deviations of 3 replicates observed either with a compound of formula (V) or (VII) treatments alone or in combination are shown in Tables 8A, 8B, 9A, and 9B as indicated below. The EC₅₀ values of a compound of formula (V) and (VII) were determined in an earlier experiment and are shown in Table 10.

When the observed values of a two-inhibitor combination were compared to what is expected from additive interaction for the above concentration range, the combination effects ranged from additive for HBsAg inhibition, with no significant synergy or antagonism, to additive to strongly synergistic for HBV DNA inhibition, as per MacSynergy II analysis and using the interpretive criteria described by Prichard and Shipman (1992) (Table 10). No significant inhibition of cell viability was observed by microscopy or CellTiter-Glo assay.

TABLE 8A
Experiment 1: Effect on HBV DNA in In Vitro Combination
of Compounds of Formula (V) and (VII)

| [DRUG] | | | | | | | AVERAGE % INHIBITION |
|---|---|---|---|---|---|---|---|
| (VII) | | | | | | | Horizontal axis: |
| μM | 0.00 | 0.04 | 0.11 | 0.33 | 1.00 | 3.00 | (V) (μg/mL) |
| 4.00 | 56.23 | 72.66 | 64.68 | 64.9 | 62.78 | 63.7 | |
| 1.33 | 53.86 | 76.94 | 73.23 | 65.06 | 71.22 | 73.59 | |
| 0.44 | 49.95 | 73.91 | 73.77 | 73.72 | 66.29 | 62.18 | |
| 0.15 | 28.72 | 65.34 | 68.48 | 69.2 | 63.87 | 65.52 | |
| 0.05 | −22.57 | 42.51 | 48.56 | 53.5 | 54.26 | 56.97 | |
| 0.00 | 0 | 29.29 | 27.51 | 38.58 | 45.67 | 38.3 | |

| [DRUG] | | | | | | | STANDARD DEVIATION (%) |
|---|---|---|---|---|---|---|---|
| (VII) | | | | | | | Horizontal axis: |
| μM | 0.00 | 0.04 | 0.11 | 0.33 | 1.00 | 3.00 | (V) (μg/mL) |
| 4.00 | 4.46 | 2.66 | 2.11 | 5.14 | 9.28 | 6.86 | |
| 1.33 | 2.55 | 2.54 | 7.86 | 14.57 | 6.51 | 1.73 | |
| 0.44 | 6.83 | 7.49 | 3.04 | 7.17 | 7.42 | 8.1 | |
| 0.15 | 2.78 | 7.31 | 2.85 | 1.55 | 5.71 | 6.32 | |
| 0.05 | 20.62 | 3.1 | 1.8 | 9.13 | 1.84 | 3.56 | |
| 0.00 | 18.08 | 4.55 | 14.58 | 6.56 | 9.76 | 6.46 | |

| [DRUG] | | | | | | | ADDITIVE % INHIBITION |
|---|---|---|---|---|---|---|---|
| (VII) | | | | | | | Horizontal axis: |
| μM | 0.00 | 0.04 | 0.11 | 0.33 | 1.00 | 3.00 | (V) (μg/mL) |
| 4.00 | 56.23 | 69.05 | 68.27 | 73.12 | 76.22 | 72.99 | |
| 1.33 | 53.86 | 67.37 | 66.55 | 71.66 | 74.93 | 71.53 | |
| 0.44 | 49.95 | 64.61 | 63.72 | 69.26 | 72.81 | 69.12 | |
| 0.15 | 28.72 | 49.6 | 48.33 | 56.22 | 61.27 | 56.02 | |
| 0.05 | −22.57 | 13.33 | 11.15 | 24.72 | 33.41 | 24.37 | |
| 0.00 | 0 | 29.29 | 27.51 | 38.58 | 45.67 | 38.3 | |

| [DRUG] | | | | | | | SYNERGY PLOT (99.9%) |
|---|---|---|---|---|---|---|---|
| (VII) | | | | | | | Bonferroni Adj. |
| μM | 0.00 | 0.04 | 0.11 | 0.33 | 1.00 | 3.00 | 98% |
| 4.00 | 0 | 0 | 0 | 0 | 0 | 0 | SYNERGY 106.05 |
| 1.33 | 0 | 1.21086 | 0 | 0 | 0 | 0 | log volume 24.14 |
| 0.44 | 0 | 0 | 0.04536 | 0 | 0 | 0 | |
| 0.15 | 0 | 0 | 10.77065 | 7.87895 | 0 | 0 | ANTAGONISM 0 |
| 0.05 | 0 | 18.9779 | 31.4862 | 0 | 14.79456 | 20.88404 | log volume 0 |
| 0.00 | 0 | 0 | 0 | 0 | 0 | 0 | |

TABLE 8B
Experiment 2: Effect on HBV DNA in In Vitro Combination
of Compounds of Formula (V) and (VII)

| [DRUG] | | | | | | | AVERAGE % INHIBITION |
|---|---|---|---|---|---|---|---|
| (VII) | | | | | | | Horizontal axis: |
| μM | 0.00 | 0.04 | 0.11 | 0.33 | 1.00 | 3.00 | (V) (μg/mL) |
| 4.00 | 41.34 | 50.29 | 53.27 | 56.26 | 50.86 | 51.83 | |
| 1.33 | 32.89 | 57.83 | 63.2 | 54.17 | 44.7 | 55.33 | |
| 0.44 | 31.22 | 48.51 | 57.57 | 53.32 | 49.38 | 50.2 | |
| 0.15 | 16.08 | 47.91 | 50.54 | 51.75 | 47.53 | 39.4 | |
| 0.05 | −0.21 | 32.62 | 48.67 | 52.47 | 47.2 | 42.14 | |
| 0.00 | 0 | 18.81 | 26.86 | 25.61 | 20.79 | 19.81 | |

| [DRUG] | | | | | | | STANDARD DEVIATION (%) |
|---|---|---|---|---|---|---|---|
| (VII) | | | | | | | Horizontal axis: |
| μM | 0.00 | 0.04 | 0.11 | 0.33 | 1.00 | 3.00 | (V) (μg/mL) |
| 4.00 | 11.22 | 6.42 | 1.81 | 4.95 | 1.17 | 9.71 | |
| 1.33 | 18.53 | 4.2 | 3.9 | 2.83 | 8.72 | 9.42 | |
| 0.44 | 11.3 | 11.8 | 10.07 | 5.59 | 5.2 | 3.37 | |
| 0.15 | 7.51 | 6.85 | 8.17 | 4.92 | 7.17 | 2.28 | |
| 0.05 | 1.69 | 11.52 | 6.82 | 4.75 | 12.13 | 5.06 | |
| 0.00 | 21.21 | 15.74 | 15.06 | 5.77 | 6.62 | 5.33 | |

| [DRUG] | | | | | | | ADDITIVE % INHIBITION |
|---|---|---|---|---|---|---|---|
| (VII) | | | | | | | Horizontal axis: |
| μM | 0.00 | 0.04 | 0.11 | 0.33 | 1.00 | 3.00 | (V) (μg/mL) |
| 4.00 | 41.34 | 52.37 | 57.1 | 56.36 | 53.54 | 52.96 | |
| 1.33 | 32.89 | 45.51 | 50.92 | 50.08 | 46.84 | 46.18 | |
| 0.44 | 31.22 | 44.16 | 49.69 | 48.83 | 45.52 | 44.85 | |
| 0.15 | 16.08 | 31.87 | 38.62 | 37.57 | 33.53 | 32.7 | |
| 0.05 | −0.21 | 18.64 | 26.71 | 25.45 | 20.62 | 19.64 | |
| 0.00 | 0 | 18.81 | 26.86 | 25.61 | 20.79 | 19.81 | |

| [DRUG] | | | | | | | SYNERGY PLOT (99.9%) |
|---|---|---|---|---|---|---|---|
| (VII) | | | | | | | Bonferroni Adj. |
| μM | 0.00 | 0.04 | 0.11 | 0.33 | 1.00 | 3.00 | 98% |
| 4.00 | 0 | 0 | 0 | 0 | 0 | 0 | SYNERGY 17.24 |
| 1.33 | 0 | 0 | 0 | 0 | 0 | 0 | log volume 3.92 |
| 0.44 | 0 | 0 | 0 | 0 | 0 | 0 | |
| 0.15 | 0 | 0 | 0 | 0 | 0 | 0 | ANTAGONISM 0 |
| 0.05 | 0 | 0 | 0 | 11.38775 | 0 | 5.84754 | log volume 0 |
| 0.00 | 0 | 0 | 0 | 0 | 0 | 0 | |

TABLE 9A
Experiment 1: Effect on HBsAg in In Vitro Combination
of Compounds of Formula (V) and (VII)

| [DRUG] | | | | | | | AVERAGE % INHIBITION |
|---|---|---|---|---|---|---|---|
| (VII) | | | | | | | Horizontal axis: |
| μM | 0.00 | 0.04 | 0.11 | 0.33 | 1.00 | 3.00 | (V) (μg/mL) |
| 4.00 | 49.65 | 79.43 | 87.66 | 90.64 | 93.46 | 95.19 | |
| 1.33 | 29.93 | 71.77 | 83.09 | 88.3 | 91.54 | 94.21 | |
| 0.44 | −9.81 | 51.47 | 71.34 | 79.49 | 86.31 | 90.53 | |
| 0.15 | −5.43 | 51.36 | 71.26 | 79.08 | 86.55 | 90.06 | |
| 0.05 | −14.08 | 53.33 | 69.55 | 79.46 | 85.8 | 89.81 | |
| 0.00 | 0 | 57.04 | 70.34 | 79.55 | 85.97 | 89.7 | |

| [DRUG] | | | | | | | STANDARD DEVIATION (%) |
|---|---|---|---|---|---|---|---|
| (VII) | | | | | | | Horizontal axis: |
| μM | 0.00 | 0.04 | 0.11 | 0.33 | 1.00 | 3.00 | (V) (μg/mL) |
| 4.00 | 1.58 | 2.23 | 0.33 | 0.65 | 0.4 | 0.17 | |
| 1.33 | 3.63 | 2.11 | 1.01 | 1.08 | 0.95 | 0.35 | |
| 0.44 | 6.27 | 3.2 | 3.81 | 0.93 | 0.95 | 2.29 | |
| 0.15 | 6.85 | 3.06 | 2.16 | 0.2 | 1.2 | 1.17 | |
| 0.05 | 10.3 | 1.2 | 1.8 | 1.3 | 0.81 | 1.18 | |
| 0.00 | 1.86 | 2.51 | 1.25 | 2.11 | 1.59 | 0.95 | |

| [DRUG] | | | | | | | ADDITIVE % INHIBITION |
|---|---|---|---|---|---|---|---|
| (VII) | | | | | | | Horizontal axis: |
| μM | 0.00 | 0.04 | 0.11 | 0.33 | 1.00 | 3.00 | (V) (μg/mL) |
| 4.00 | 49.65 | 78.37 | 85.07 | 89.7 | 92.94 | 94.81 | |
| 1.33 | 29.93 | 69.9 | 79.22 | 85.67 | 90.17 | 92.78 | |
| 0.44 | −9.81 | 52.83 | 67.43 | 77.54 | 84.59 | 88.69 | |
| 0.15 | −5.43 | 54.71 | 68.73 | 78.44 | 85.21 | 89.14 | |
| 0.05 | −14.08 | 50.99 | 66.16 | 76.67 | 83.99 | 88.25 | |
| 0.00 | 0 | 57.04 | 70.34 | 79.55 | 85.97 | 89.7 | |

| [DRUG] | | | | | | | SYNERGY PLOT (99.9%) |
|---|---|---|---|---|---|---|---|
| (VII) | | | | | | | Bonferroni Adj. |
| μM | 0.00 | 0.04 | 0.11 | 0.33 | 1.00 | 3.00 | 98% |
| 4.00 | 0 | 0 | 1.50397 | 0 | 0 | 0 | SYNERGY 2.33 |
| 1.33 | 0 | 0 | 0.54609 | 0 | 0 | 0.27815 | log volume 0.53 |
| 0.44 | 0 | 0 | 0 | 0 | 0 | 0 | |
| 0.15 | 0 | 0 | 0 | 0 | 0 | 0 | ANTAGONISM 0 |
| 0.05 | 0 | 0 | 0 | 0 | 0 | 0 | log volume 0 |
| 0.00 | 0 | 0 | 0 | 0 | 0 | 0 | |

TABLE 9B
Experiment 2: Effect on HBsAg in In Vitro Combination
of Compounds of Formula (V) and (VII)

| [DRUG] | | | | | | | AVERAGE % INHIBITION |
|---|---|---|---|---|---|---|---|
| (VII) | | | | | | | Horizontal axis: |
| μM | 0.00 | 0.04 | 0.11 | 0.33 | 1.00 | 3.00 | (V) (μg/mL) |
| 4.00 | 48.66 | 80.78 | 87.37 | 92.43 | 94.79 | 96.3 | |
| 1.33 | 29.39 | 74.38 | 83.63 | 90.07 | 93.16 | 95.14 | |
| 0.44 | 1.73 | 62.63 | 75.26 | 84.94 | 88.73 | 92.47 | |
| 0.15 | −8.51 | 59.3 | 74.14 | 84.81 | 88.26 | 92.21 | |
| 0.05 | −3.42 | 58.5 | 73.62 | 83.54 | 88.49 | 91.9 | |
| 0.00 | 0 | 64.35 | 78.29 | 86.27 | 89.88 | 93.05 | |

| [DRUG] | | | | | | | STANDARD DEVIATION (%) |
|---|---|---|---|---|---|---|---|
| (VII) | | | | | | | Horizontal axis: |
| μM | 0.00 | 0.04 | 0.11 | 0.33 | 1.00 | 3.00 | (V) (μg/mL) |
| 4.00 | 4.22 | 1.75 | 0.97 | 0.59 | 0.19 | 0.16 | |
| 1.33 | 3.27 | 2.6 | 1 | 0.79 | 0.12 | 0.28 | |
| 0.44 | 2.36 | 5.46 | 2.67 | 1.46 | 0.7 | 0.32 | |
| 0.15 | 6.06 | 6.09 | 3.64 | 2.18 | 1.39 | 0.32 | |
| 0.05 | 5.19 | 4.57 | 1.88 | 1.11 | 1.8 | 0.63 | |
| 0.00 | 6.44 | 1.47 | 2.06 | 1.43 | 0.21 | 0.18 | |

| [DRUG] | | | | | | | ADDITIVE % INHIBITION |
|---|---|---|---|---|---|---|---|
| (VII) | | | | | | | Horizontal axis: |
| μM | 0.00 | 0.04 | 0.11 | 0.33 | 1.00 | 3.00 | (V) (μg/mL) |
| 4.00 | 48.66 | 81.7 | 88.85 | 92.95 | 94.8 | 96.43 | |
| 1.33 | 29.39 | 74.83 | 84.67 | 90.31 | 92.85 | 95.09 | |
| 0.44 | 1.73 | 64.97 | 78.67 | 86.51 | 90.06 | 93.17 | |
| 0.15 | −8.51 | 61.32 | 76.44 | 85.1 | 89.02 | 92.46 | |
| 0.05 | −3.42 | 63.13 | 77.55 | 85.8 | 89.53 | 92.81 | |
| 0.00 | 0 | 64.35 | 78.29 | 86.27 | 89.88 | 93.05 | |

| [DRUG] | | | | | | | SYNERGY PLOT (99.9%) |
|---|---|---|---|---|---|---|---|
| (VII) | | | | | | | Bonferroni Adj. |
| μM | 0.00 | 0.04 | 0.11 | 0.33 | 1.00 | 3.00 | 98% |
| 4.00 | 0 | 0 | 0 | 0 | 0 | 0 | SYNERGY 0 |
| 1.33 | 0 | 0 | 0 | 0 | 0 | 0 | log volume 0 |
| 0.44 | 0 | 0 | 0 | 0 | 0 | 0 | |
| 0.15 | 0 | 0 | 0 | 0 | 0 | 0 | ANTAGONISM 0 |
| 0.05 | 0 | 0 | 0 | 0 | 0 | 0 | log volume 0 |
| 0.00 | 0 | 0 | 0 | 0 | 0 | 0 | |

TABLE 10
Summary of results of in vitro combination studies of compounds
of Formula (V) and (VII) in PHH cell culture system:

| HBV | (VII) | (V) | | Synergy | | Antagonism | |
|---|---|---|---|---|---|---|---|
| Assay | EC₅₀ | EC₅₀ | Synergy | Log | Antagonism | Log | |
| Endpoint | (μM)# | (μg/mL)# | Volume* | Volume* | Volume* | Volume* | Conclusion |
| HBV | 0.076 | <0.123 | 106.05, 17.24 | 24.14, 3.92 | 0, 0 | 0, 0 | Additive to |
| DNA | | | | | | | Strong Synergy |
| HBsAg | >4.0 | <0.123 | 2.33, 0 | 0.53,0 | 0, 0 | 0, 0 | Additive |

*at 99.9% confidence interval
#determined in an earlier separate experiment

### Example 3. In Vitro Combination of a Compound of Formula (V) and PEG-IFNα2a

### Study Goal

To determine whether a two-drug combination of a compound of formula (V) (a GalNAc-conjugated siRNA targeting the HBV genome, and inhibiting production of HBV DNA, HBsAg and HBeAg, and HBx), and pegylated interferon alpha 2a (PEG-IFNα2a, an antiviral cytokine that activates innate immunity pathways in hepatocytes, and is used clinically for treatment of chronic hepatitis B), is additive, synergistic or antagonistic in vitro using HBV-infected human primary hepatocytes in a cell culture model system.

### Results and Conclusion

PEG-IFNα2a (concentration range of 80.0 IU/mL to 0.99 IU/mL in a 3-fold dilution series and 5-point titration) was tested in combination with a compound of formula (V) (concentration range of 3.0 μg/mL to 0.04 μg/mL in a 3-fold dilution series and 5-point titration), on three replicate plates in each of two separate experimental trials. The average % inhibition in HBV DNA and HBsAg, % standard deviations of 3 replicate plates, average additive % inhibition, and synergy/antagonism volumes observed either with PEG-IFNα2a or compound of formula (V) treatments alone or in combination are shown in Tables 11A, 11B, 12A, and 12B as indicated below. The EC₅₀ values of PEG-IFNα2a and compound of formula (V) were determined in an earlier experiment and are shown in Table 13.

When the observed values of a two-inhibitor combination were compared to what is expected from additive interaction by calculation of synergy/antagonism volumes, the combination effects were found to be additive for both HBsAg and HBV DNA inhibition, with no significant synergy or antagonism, as per MacSynergy II analysis, and using the interpretive criteria described by Prichard and Shipman (1992) (Table 13). No significant inhibition of cell viability was observed by microscopy or CellTiter-Glo assay.

TABLE 11A
Experiment 1: Effect on HBV DNA in In Vitro Combination
of a Compound of Formula (V) and PEG-IFNα2a

| [DRUG] | | | | | | | AVERAGE % INHIBITION |
|---|---|---|---|---|---|---|---|
| PEG-IFNα2a | | | | | | | Horizontal axis: |
| IU/mL | 0.00 | 0.04 | 0.11 | 0.33 | 1.00 | 3.00 | (V) (μg/mL) |
| 80.00 | 62.5 | 62.68 | 65.57 | 61.43 | 71.42 | 63.11 | |
| 26.67 | 66.76 | 73.19 | 74.69 | 72.86 | 79.34 | 74.56 | |
| 8.89 | 58.14 | 76.67 | 79.13 | 79.76 | 80.33 | 74.91 | |
| 2.96 | 55.19 | 68.23 | 74.57 | 73.89 | 73.56 | 76.04 | |
| 0.99 | 39.75 | 57.21 | 64.65 | 62.01 | 67.15 | 66.11 | |
| 0.00 | 0 | 30.89 | 41.28 | 49.43 | 55.63 | 55.15 | |
| [DRUG] | | | | | | | STANDARD DEVIATION (%) |
| PEG-IFNα2a | | | | | | | Horizontal axis: |
| IU/mL | 0.00 | 0.04 | 0.11 | 0.33 | 1.00 | 3.00 | (V) (μg/mL) |
| 80.00 | 8.13 | 3.37 | 4.97 | 3.16 | 3.63 | 8.74 | |
| 26.67 | 6.49 | 1.42 | 3.67 | 5.13 | 0.88 | 3.73 | |
| 8.89 | 10.25 | 3.95 | 6.7 | 2.29 | 1.9 | 2.68 | |
| 2.96 | 4.31 | 8.79 | 5.32 | 3.43 | 5.4 | 0.77 | |
| 0.99 | 9.43 | 4.25 | 2.34 | 8.77 | 3.86 | 1.58 | |
| 0.00 | 39.07 | 8.47 | 10.87 | 9.72 | 1.27 | 7.34 | |
| [DRUG] | | | | | | | ADDITIVE % INHIBITION |
| PEG-IFNα2a | | | | | | | Horizontal axis: |
| IU/mL | 0.00 | 0.04 | 0.11 | 0.33 | 1.00 | 3.00 | (V) (μg/mL) |
| 80.00 | 62.5 | 74.08 | 77.98 | 81.04 | 83.36 | 83.18 | |
| 26.67 | 66.76 | 77.03 | 80.48 | 83.19 | 85.25 | 85.09 | |
| 8.89 | 58.14 | 71.07 | 75.42 | 78.83 | 81.43 | 81.23 | |
| 2.96 | 55.19 | 69.03 | 73.69 | 77.34 | 80.12 | 79.9 | |
| 0.99 | 39.75 | 58.36 | 64.62 | 69.53 | 73.27 | 72.98 | |
| 0.00 | 0 | 30.89 | 41.28 | 49.43 | 55.63 | 55.15 | |

| [DRUG] | | | | | | | SYNERGY PLOT (99.9%) |
|---|---|---|---|---|---|---|---|
| PEG-IFNα2a | | | | | | | Bonferroni Adj. |
| IU/mL | 0.00 | 0.04 | 0.11 | 0.33 | 1.00 | 3.00 | 98% |
| 80.00 | 0 | −0.30933 | 0 | −9.21044 | 0 | 0 | SYNERGY 0 |
| 26.67 | 0 | 0 | 0 | 0 | −3.01392 | 0 | log volume 0 |
| 8.89 | 0 | 0 | 0 | 0 | 0 | 0 | |
| 2.96 | 0 | 0 | 0 | 0 | 0 | −1.32593 | ANTAGONISM −15.53 |
| 0.99 | 0 | 0 | 0 | 0 | 0 | −1.67022 | log volume −3.54 |
| 0.00 | 0 | 0 | 0 | 0 | 0 | 0 | |

TABLE 11B
Experiment 2: Effect on HBV DNA in In Vitro Combination
of a Compound of Formula (V) and PEG-IFNα2a

| [DRUG] | | | | | | | AVERAGE % INHIBITION |
|---|---|---|---|---|---|---|---|
| PEG-IFNα2a | | | | | | | Horizontal axis: |
| IU/mL | 0.00 | 0.04 | 0.11 | 0.33 | 1.00 | 3.00 | (V) (μg/mL) |
| 80.00 | 44.32 | 53.56 | 49.81 | 50.79 | 56.36 | 52.58 | |
| 26.67 | 30.34 | 56 | 61.33 | 56.31 | 48.71 | 48.26 | |
| 8.89 | 23.55 | 63.29 | 69.37 | 61.12 | 53.33 | 54.34 | |
| 2.96 | 21.57 | 54.44 | 60.9 | 50.29 | 49.93 | 46.4 | |
| 0.99 | 21.02 | 44.24 | 60.81 | 62.27 | 49.04 | 48.62 | |
| 0.00 | 0 | 24.99 | 28.01 | 38.63 | 43.89 | 39.52 | |

| [DRUG] | | | | | | | STANDARD DEVIATION (%) |
|---|---|---|---|---|---|---|---|
| PEG-IFNα2a | | | | | | | Horizontal axis: |
| IU/mL | 0.00 | 0.04 | 0.11 | 0.33 | 1.00 | 3.00 | (V) (μg/mL) |
| 80.00 | 17.29 | 12.55 | 16.24 | 15.83 | 12 | 16.94 | |
| 26.67 | 10.4 | 7.33 | 2.98 | 5.82 | 3.65 | 3.48 | |
| 8.89 | 16.34 | 4.17 | 8 | 7.56 | 7.58 | 3.82 | |
| 2.96 | 13.02 | 4.44 | 7.59 | 2.48 | 8.47 | 5.89 | |
| 0.99 | 11.57 | 19.07 | 8.19 | 4.87 | 13.45 | 8.33 | |
| 0.00 | 12.93 | 2.87 | 34.4 | 11.34 | 9.62 | 9.14 | |

| [DRUG] | | | | | | | ADDITIVE % INHIBITION |
|---|---|---|---|---|---|---|---|
| PEG-IFNα2a | | | | | | | Horizontal axis: |
| IU/mL | 0.00 | 0.04 | 0.11 | 0.33 | 1.00 | 3.00 | (V) (μg/mL) |
| 80.00 | 44.32 | 58.23 | 59.92 | 65.83 | 68.76 | 66.32 | |
| 26.67 | 30.34 | 47.75 | 49.85 | 57.25 | 60.91 | 57.87 | |
| 8.89 | 23.55 | 42.65 | 44.96 | 53.08 | 57.1 | 53.76 | |
| 2.96 | 21.57 | 41.17 | 43.54 | 51.87 | 55.99 | 52.57 | |
| 0.99 | 21.02 | 40.76 | 43.14 | 51.53 | 55.68 | 52.23 | |
| 0.00 | 0 | 24.99 | 28.01 | 38.63 | 43.89 | 39.52 | |

| [DRUG] | | | | | | | SYNERGY PLOT (99.9%) |
|---|---|---|---|---|---|---|---|
| PEG-IFNα2a | | | | | | | Bonferroni Adj. |
| IU/mL | 0.00 | 0.04 | 0.11 | 0.33 | 1.00 | 3.00 | 98% |
| 80.00 | 0 | 0 | 0 | 0 | 0 | 0 | SYNERGY 8.59 |
| 26.67 | 0 | 0 | 1.67282 | 0 | −0.18785 | 0 | log volume 1.96 |
| 8.89 | 0 | 6.91653 | 0 | 0 | 0 | 0 | |
| 2.96 | 0 | 0 | 0 | 0 | 0 | 0 | ANTAGONISM −0.19 |
| 0.99 | 0 | 0 | 0 | 0 | 0 | 0 | log volume −0.04 |
| 0.00 | 0 | 0 | 0 | 0 | 0 | 0 | |

TABLE 12A
Experiment 1: Effect on HBsAg in In Vitro Combination
of a Compound of Formula (V) and PEG-IFNα2a

| [DRUG] | | | | | | | AVERAGE % INHIBITION |
|---|---|---|---|---|---|---|---|
| PEG-IFNα2a | | | | | | | Horizontal axis: |
| IU/mL | 0.00 | 0.04 | 0.11 | 0.33 | 1.00 | 3.00 | (V) (μg/mL) |
| 80.00 | 99.4 | 99.66 | 99.82 | 99.87 | 99.91 | 99.96 | |
| 26.67 | 97.1 | 98.35 | 99.1 | 99.3 | 99.56 | 99.7 | |
| 8.89 | 89.06 | 94.27 | 96.61 | 97.5 | 98.38 | 98.88 | |
| 2.96 | 75.32 | 86.32 | 92.14 | 93.97 | 96.06 | 97.36 | |
| 0.99 | 52.53 | 76.35 | 85.78 | 89.64 | 93.48 | 95.23 | |
| 0.00 | 0 | 57.79 | 71.54 | 80.32 | 87.03 | 90.18 | |
| [DRUG] | | | | | | | STANDARD DEVIATION (%) |
| PEG-IFNα2a | | | | | | | Horizontal axis: |
| IU/mL | 0.00 | 0.04 | 0.11 | 0.33 | 1.00 | 3.00 | (V) (μg/mL) |
| 80.00 | 0.47 | 0.15 | 0.11 | 0.1 | 0.08 | 0.06 | |
| 26.67 | 0.84 | 0.44 | 0.29 | 0.27 | 0.24 | 0.14 | |
| 8.89 | 2.83 | 1.13 | 1.11 | 0.54 | 0.59 | 0.32 | |
| 2.96 | 5.1 | 2.28 | 1.39 | 1.16 | 0.78 | 0.59 | |
| 0.99 | 6.99 | 2.37 | 2.88 | 1.69 | 1.37 | 0.7 | |
| 0.00 | 13.25 | 3.83 | 3.52 | 0.67 | 0.9 | 0.84 | |
| [DRUG] | | | | | | | ADDITIVE % INHIBITION |
| PEG-IFNα2a | | | | | | | Horizontal axis: |
| IU/mL | 0.00 | 0.04 | 0.11 | 0.33 | 1.00 | 3.00 | (V) (μg/mL) |
| 80.00 | 99.4 | 99.75 | 99.83 | 99.88 | 99.92 | 99.94 | |
| 26.67 | 97.1 | 98.78 | 99.17 | 99.43 | 99.62 | 99.72 | |
| 8.89 | 89.06 | 95.38 | 96.89 | 97.85 | 98.58 | 98.93 | |
| 2.96 | 75.32 | 89.58 | 92.98 | 95.14 | 96.8 | 97.58 | |
| 0.99 | 52.53 | 79.96 | 86.49 | 90.66 | 93.84 | 95.34 | |
| 0.00 | 0 | 57.79 | 71.54 | 80.32 | 87.03 | 90.18 | |

| [DRUG] | | | | | | | SYNERGY PLOT (99.9%) |
|---|---|---|---|---|---|---|---|
| PEG-IFNα2a | | | | | | | Bonferroni Adj. |
| IU/mL | 0.00 | 0.04 | 0.11 | 0.33 | 1.00 | 3.00 | 98% |
| 80.00 | 0 | 0 | 0 | 0 | 0 | 0 | SYNERGY 0 |
| 26.67 | 0 | 0 | 0 | 0 | 0 | 0 | log volume 0 |
| 8.89 | 0 | 0 | 0 | 0 | 0 | 0 | |
| 2.96 | 0 | 0 | 0 | 0 | 0 | 0 | ANTAGONISM 0 |
| 0.99 | 0 | 0 | 0 | 0 | 0 | 0 | log volume 0 |
| 0.00 | 0 | 0 | 0 | 0 | 0 | 0 | |

TABLE 12B
Experiment 2: Effect on HBsAg in In Vitro Combination
of a Compound of Formula (V) and PEG-IFNα2a

| [DRUG] | | | | | | | AVERAGE % INHIBITION |
|---|---|---|---|---|---|---|---|
| PEG-IFNα2a | | | | | | | Horizontal axis: |
| IU/mL | 0.00 | 0.04 | 0.11 | 0.33 | 1.00 | 3.00 | (V) (μg/mL) |
| 80.00 | 98.98 | 99.71 | 99.82 | 99.92 | 99.93 | 99.97 | |
| 26.67 | 97.29 | 99.08 | 99.46 | 99.69 | 99.8 | 99.91 | |
| 8.89 | 94.15 | 97.48 | 98.56 | 99.13 | 99.44 | 99.58 | |
| 2.96 | 85.57 | 93.5 | 95.87 | 97.32 | 98.23 | 98.76 | |
| 0.99 | 72.45 | 87.77 | 91.84 | 94.97 | 96.51 | 97.42 | |
| 0.00 | 0 | 69.5 | 80.38 | 86.88 | 91.51 | 93.77 | |

| [DRUG] | | | | | | | STANDARD DEVIATION (%) |
|---|---|---|---|---|---|---|---|
| PEG-IFNα2a | | | | | | | Horizontal axis: |
| IU/mL | 0.00 | 0.04 | 0.11 | 0.33 | 1.00 | 3.00 | (V) (μg/mL) |
| 80.00 | 0.02 | 0.03 | 0.06 | 0.01 | 0.03 | 0.03 | |
| 26.67 | 0.19 | 0.16 | 0.06 | 0.05 | 0.04 | 0.03 | |
| 8.89 | 0.55 | 0.31 | 0.17 | 0.21 | 0.15 | 0.13 | |
| 2.96 | 1.5 | 0.46 | 0.25 | 0.54 | 0.06 | 0.22 | |
| 0.99 | 4.41 | 1.81 | 0.43 | 0.54 | 0.46 | 0.42 | |
| 0.00 | 4.4 | 3.15 | 1.15 | 0.94 | 0.7 | 0.57 | |

| [DRUG] | | | | | | | ADDITIVE % INHIBITION |
|---|---|---|---|---|---|---|---|
| PEG-IFNα2a | | | | | | | Horizontal axis: |
| IU/mL | 0.00 | 0.04 | 0.11 | 0.33 | 1.00 | 3.00 | (V) (μg/mL) |
| 80.00 | 98.98 | 99.69 | 99.8 | 99.87 | 99.91 | 99.94 | |
| 26.67 | 97.29 | 99.17 | 99.47 | 99.64 | 99.77 | 99.83 | |
| 8.89 | 94.15 | 98.22 | 98.85 | 99.23 | 99.5 | 99.64 | |
| 2.96 | 85.57 | 95.6 | 97.17 | 98.11 | 98.77 | 99.1 | |
| 0.99 | 72.45 | 91.6 | 94.59 | 96.39 | 97.66 | 98.28 | |
| 0.00 | 0 | 69.5 | 80.38 | 86.88 | 91.51 | 93.77 | |

| [DRUG] | | | | | | | SYNERGY PLOT (99.9%) |
|---|---|---|---|---|---|---|---|
| PEG-IFNα2a | | | | | | | Bonferroni Adj. |
| IU/mL | 0.00 | 0.04 | 0.11 | 0.33 | 1.00 | 3.00 | 98% |
| 80.00 | 0 | 0 | 0 | 0.01709 | 0 | 0 | SYNERGY 0.02 |
| 26.67 | 0 | 0 | 0 | 0 | 0 | 0 | log volume 0 |
| 8.89 | 0 | 0 | 0 | 0 | 0 | 0 | |
| 2.96 | 0 | −0.58614 | −0.47725 | 0 | −0.34254 | 0 | ANTAGONISM −2.74 |
| 0.99 | 0 | 0 | −1.33487 | 0 | 0 | 0 | log volume −0.62 |
| 0.00 | 0 | 0 | 0 | 0 | 0 | 0 | |

TABLE 13
Summary of results of in vitro combination studies of PEG-IFNα2a
and a Compound of Formula (V) in PHH cell culture system:

| | PEG- | | | | | | |
|---|---|---|---|---|---|---|---|
| HBV | IFNα2a | (V) | | Synergy | | Antagonism | |
| Assay | EC₅₀ | EC₅₀ | Synergy | Log | Antagonism | Log | |
| Endpoint | (IU/mL)# | (μg/mL)# | Volume* | Volume* | Volume * | Volume* | Conclusion |
| HBV DNA | 1.192 | <0.123 | 0, 8.59 | 0, 1.96 | −15.53, −0.19 | −3.54, −0.04 | Additive |
| HBsAg | 12.910 | <0.123 | 0, 0.02 | 0, 0 | 0, −2.74 | 0, −0.62 | Additive |

*at 99.9% confidence interval
#determined in an earlier separate experiment

### Example 4. In Vitro Combination of a Compound of Formula (V) and TAF

### Study Goal

To determine whether a two-drug combination of a compound of formula (V) (a GalNAc-conjugated siRNA targeting the HBV genome, and inhibiting production of HBV DNA, HBsAg and HBeAg, and HBx), and tenofovir alafenamide fumarate (TAF, a nucleoside analogue that inhibits the HBV reverse transcriptase enzyme, and is used clinically for treatment of chronic hepatitis B), is additive, synergistic or antagonistic in vitro, using HBV-infected human primary hepatocytes in a cell culture model system.

### Results and Conclusion

TAF (concentration range of 1.000 nM to 0.012 nM in a 3-fold dilution series and 5-point titration) was tested in combination with a compound of formula (V) (concentration range of 3.0 μg/mL to 0.04 μg/mL in a 3-fold dilution series and 5-point titration), on three replicate plates in each of two separate experimental trials. The average % inhibition in HBV DNA and

HBsAg, and standard deviations of 3 replicates observed either with TAF or compound of formula (V) treatments alone or in combination are shown in Tables 14A, 14B, 15A, and 15B as indicated below. The EC₅₀ values of TAF and compound of formula (V) were determined in an earlier experiment and are shown in Table 16.

When the observed values of a two-inhibitor combination were compared to what is expected from additive interaction for the above concentration range, the combination effects ranged from additive for HBsAg inhibition, with no significant synergy or antagonism, to additive to moderately synergistic for HBV DNA inhibition, as per MacSynergy II analysis, and using the interpretive criteria described by Prichard and Shipman (1992) (Table 16). No significant inhibition of cell viability was observed by microscopy or CellTiter-Glo assay.

TABLE 14A
Experiment 1: Effect on HBV DNA in In Vitro Combination
of a Compound of Formula (V) and TAF

| [DRUG] | | | | | | | AVERAGE % INHIBITION |
|---|---|---|---|---|---|---|---|
| TAF | | | | | | | Horizontal axis: |
| nM | 0.00 | 0.04 | 0.11 | 0.33 | 1.00 | 3.00 | (V) (μg/mL) |
| 1.00 | 46.57 | 48.75 | 47.58 | 60.32 | 60.22 | 57.82 | |
| 0.33 | 53.11 | 66.86 | 57.27 | 68.01 | 64.44 | 64.65 | |
| 0.11 | 40.21 | 60.1 | 59.51 | 66.43 | 67.92 | 63.07 | |
| 0.04 | 3.33 | 39.75 | 40.13 | 56.21 | 57.34 | 58.09 | |
| 0.01 | −31.65 | 20.48 | 33.52 | 42.17 | 48.39 | 52.64 | |
| 0.00 | 0 | −5.95 | 10.67 | 24.72 | 39.81 | 46.3 | |

| [DRUG] | | | | | | | STANDARD DEVIATION (%) |
|---|---|---|---|---|---|---|---|
| TAF | | | | | | | Horizontal axis: |
| nM | 0.00 | 0.04 | 0.11 | 0.33 | 1.00 | 3.00 | (V) (μg/mL) |
| 1.00 | 6.66 | 13.37 | 2.56 | 10.67 | 11.17 | 7.47 | |
| 0.33 | 8.8 | 3.61 | 22.86 | 7.48 | 3.73 | 5.07 | |
| 0.11 | 3.11 | 6.64 | 15.16 | 8.23 | 4.41 | 1.13 | |
| 0.04 | 22.53 | 10.4 | 14.95 | 11.33 | 9.03 | 9.99 | |
| 0.01 | 25.37 | 5.24 | 12.32 | 11.47 | 5.38 | 4.82 | |
| 0.00 | 37.15 | 10.96 | 19.19 | 13.94 | 5.66 | 5.69 | |

| [DRUG] | | | | | | | ADDITIVE % INHIBITION |
|---|---|---|---|---|---|---|---|
| TAF | | | | | | | Horizontal axis: |
| nM | 0.00 | 0.04 | 0.11 | 0.33 | 1.00 | 3.00 | (V) (μg/mL) |
| 1.00 | 46.57 | 43.39 | 52.27 | 59.78 | 67.84 | 71.31 | |
| 0.33 | 53.11 | 50.32 | 58.11 | 64.7 | 71.78 | 74.82 | |
| 0.11 | 40.21 | 36.65 | 46.59 | 54.99 | 64.01 | 67.89 | |
| 0.04 | 3.33 | −2.42 | 13.64 | 27.23 | 41.81 | 48.09 | |
| 0.01 | −31.65 | −39.48 | −17.6 | 0.89 | 20.76 | 29.3 | |
| 0.00 | 0 | −5.95 | 10.67 | 24.72 | 39.81 | 46.3 | |

| [DRUG] | | | | | | | SYNERGY PLOT (99.9%) |
|---|---|---|---|---|---|---|---|
| TAF | | | | | | | Bonferroni Adj. |
| nM | 0.00 | 0.04 | 0.11 | 0.33 | 1.00 | 3.00 | 98% |
| 1.00 | 0 | 0 | 0 | 0 | 0 | 0 | SYNERGY 88.42 |
| 0.33 | 0 | 4.65949 | 0 | 0 | 0 | 0 | log volume 20.13 |
| 0.11 | 0 | 1.59776 | 0 | 0 | 0 | −1.10117 | |
| 0.04 | 0 | 7.9436 | 0 | 0 | 0 | 0 | ANTAGONISM −1.1 |
| 0.01 | 0 | 42.71516 | 10.57488 | 3.53223 | 9.92442 | 7.47738 | log volume −0.25 |
| 0.00 | 0 | 0 | 0 | 0 | 0 | 0 | |

TABLE 14B
Experiment 2: Effect on HBV DNA in In Vitro Combination
of a Compound of Formula (V) and TAF

| [DRUG] | | | | | | | AVERAGE % INHIBITION |
|---|---|---|---|---|---|---|---|
| TAF | | | | | | | Horizontal axis: |
| nM | 0.00 | 0.04 | 0.11 | 0.33 | 1.00 | 3.00 | (V) (μg/mL) |
| 1.00 | 28.87 | 41.24 | 44.81 | 53.53 | 43.73 | 46.24 | |
| 0.33 | 31.27 | 52.85 | 49.42 | 55.44 | 52.47 | 46.26 | |
| 0.11 | 29.63 | 51.88 | 49.83 | 55.88 | 49.98 | 52.1 | |
| 0.04 | 4.44 | 34.69 | 37.11 | 47.38 | 47.65 | 44.97 | |
| 0.01 | 0.33 | 21.08 | 30 | 41.62 | 38.54 | 40.74 | |
| 0.00 | 0 | 16.42 | 33.77 | 40.97 | 34.08 | 39.12 | |

| [DRUG] | | | | | | | STANDARD DEVIATION (%) |
|---|---|---|---|---|---|---|---|
| TAF | | | | | | | Horizontal axis: |
| nM | 0.00 | 0.04 | 0.11 | 0.33 | 1.00 | 3.00 | (V) (μg/mL) |
| 1.00 | 19.05 | 4.84 | 2.19 | 5.06 | 6.41 | 2.83 | |
| 0.33 | 8.6 | 6.43 | 13.62 | 11.03 | 6.7 | 3.99 | |
| 0.11 | 4.85 | 6.26 | 7.1 | 0.69 | 1.87 | 8.23 | |
| 0.04 | 5.15 | 3.97 | 6.59 | 13.73 | 2.94 | 17.52 | |
| 0.01 | 14.55 | 15.14 | 5.92 | 2.01 | 4.44 | 9.35 | |
| 0.00 | 8.18 | 19.1 | 8.21 | 3.97 | 7.51 | 7.75 | |

| [DRUG] | | | | | | | ADDITIVE % INHIBITION |
|---|---|---|---|---|---|---|---|
| TAF | | | | | | | Horizontal axis: |
| nM | 0.00 | 0.04 | 0.11 | 0.33 | 1.00 | 3.00 | (V) (μg/mL) |
| 1.00 | 28.87 | 40.55 | 52.89 | 58.01 | 53.11 | 56.7 | |
| 0.33 | 31.27 | 42.56 | 54.48 | 59.43 | 54.69 | 58.16 | |
| 0.11 | 29.63 | 41.18 | 53.39 | 58.46 | 53.61 | 57.16 | |
| 0.04 | 4.44 | 20.13 | 36.71 | 43.59 | 37.01 | 41.82 | |
| 0.01 | 0.33 | 16.7 | 33.99 | 41.16 | 34.3 | 39.32 | |
| 0.00 | 0 | 16.42 | 33.77 | 40.97 | 34.08 | 39.12 | |

| [DRUG] | | | | | | | SYNERGY PLOT (99.9%) |
|---|---|---|---|---|---|---|---|
| TAF | | | | | | | Bonferroni Adj. |
| nM | 0.00 | 0.04 | 0.11 | 0.33 | 1.00 | 3.00 | 98% |
| 1.00 | 0 | 0 | −0.87271 | 0 | 0 | −1.14647 | SYNERGY 2.46 |
| 0.33 | 0 | 0 | 0 | 0 | 0 | 0 | log volume 0.56 |
| 0.11 | 0 | 0 | 0 | −0.30921 | 0 | 0 | |
| 0.04 | 0 | 1.49473 | 0 | 0 | 0.96446 | 0 | ANTAGONISM −2.33 |
| 0.01 | 0 | 0 | 0 | 0 | 0 | 0 | log volume −0.53 |
| 0.00 | 0 | 0 | 0 | 0 | 0 | 0 | |

TABLE 15A
Experiment 1: Effect on HBsAg in In Vitro Combination
of a Compound of Formula (V) and TAF

| [DRUG] | | | | | | | AVERAGE % INHIBITION |
|---|---|---|---|---|---|---|---|
| TAF | | | | | | | Horizontal axis: |
| nM | 0.00 | 0.04 | 0.11 | 0.33 | 1.00 | 3.00 | (V) (μg/mL) |
| 1.00 | 33.95 | 66.22 | 76.42 | 84.97 | 89.04 | 92.19 | |
| 0.33 | 15.11 | 59.62 | 72.78 | 81.8 | 87.21 | 90.57 | |
| 0.11 | 2.59 | 51.88 | 69.29 | 79.65 | 85.25 | 89.79 | |
| 0.04 | 2.1 | 51.7 | 67.98 | 77.99 | 84.71 | 89.44 | |
| 0.01 | −2.28 | 50.42 | 66.87 | 77.46 | 84.27 | 88.77 | |
| 0.00 | 0 | 54.73 | 69.01 | 78.82 | 84.67 | 89.23 | |

| [DRUG] | | | | | | | STANDARD DEVIATION (%) |
|---|---|---|---|---|---|---|---|
| TAF | | | | | | | Horizontal axis: |
| nM | 0.00 | 0.04 | 0.11 | 0.33 | 1.00 | 3.00 | (V) (μg/mL) |
| 1.00 | 3.91 | 5.41 | 3.52 | 1.87 | 1.09 | 0.84 | |
| 0.33 | 6.21 | 4.42 | 3.15 | 1.3 | 1.3 | 0.94 | |
| 0.11 | 6.36 | 2.88 | 3.17 | 2.13 | 1.35 | 0.64 | |
| 0.04 | 4.16 | 1 | 0.15 | 2.24 | 1.52 | 0.93 | |
| 0.01 | 10.79 | 0.82 | 1.81 | 1.1 | 1.43 | 1.14 | |
| 0.00 | 7.24 | 6.71 | 1.64 | 0.51 | 0.46 | 0.44 | |

| [DRUG] | | | | | | | ADDITIVE % INHIBITION |
|---|---|---|---|---|---|---|---|
| TAF | | | | | | | Horizontal axis: |
| nM | 0.00 | 0.04 | 0.11 | 0.33 | 1.00 | 3.00 | (V) (μg/mL) |
| 1.00 | 33.95 | 70.1 | 79.53 | 86.01 | 89.87 | 92.89 | |
| 0.33 | 15.11 | 61.57 | 73.69 | 82.02 | 86.99 | 90.86 | |
| 0.11 | 2.59 | 55.9 | 69.81 | 79.37 | 85.07 | 89.51 | |
| 0.04 | 2.1 | 55.68 | 69.66 | 79.26 | 84.99 | 89.46 | |
| 0.01 | −2.28 | 53.7 | 68.3 | 78.34 | 84.32 | 88.98 | |
| 0.00 | 0 | 54.73 | 69.01 | 78.82 | 84.67 | 89.23 | |

| [DRUG] | | | | | | | SYNERGY PLOT (99.9%) |
|---|---|---|---|---|---|---|---|
| TAF | | | | | | | Bonferroni Adj. |
| nM | 0.00 | 0.04 | 0.11 | 0.33 | 1.00 | 3.00 | 98% |
| 1.00 | 0 | 0 | 0 | 0 | 0 | 0 | SYNERGY 0 |
| 0.33 | 0 | 0 | 0 | 0 | 0 | 0 | log volume 0 |
| 0.11 | 0 | 0 | 0 | 0 | 0 | 0 | |
| 0.04 | 0 | −0.689 | −1.18635 | 0 | 0 | 0 | ANTAGONISM −2.46 |
| 0.01 | 0 | −0.58138 | 0 | 0 | 0 | 0 | log volume −0.56 |
| 0.00 | 0 | 0 | 0 | 0 | 0 | 0 | |

TABLE 15B
Experiment 2: Effect on HBsAg in In Vitro Combination
of a Compound of Formula (V) and TAF

| [DRUG] | | | | | | | AVERAGE % INHIBITION |
|---|---|---|---|---|---|---|---|
| TAF | | | | | | | Horizontal axis: |
| nM | 0.00 | 0.04 | 0.11 | 0.33 | 1.00 | 3.00 | (V) (μg/mL) |
| 1.00 | 29.9 | 70.8 | 79.13 | 87.16 | 90.89 | 93.65 | |
| 0.33 | 12.91 | 64.87 | 75.63 | 84.83 | 88.63 | 91.85 | |
| 0.11 | −4.19 | 59.48 | 72.08 | 83.43 | 87.67 | 90.69 | |
| 0.04 | −2.84 | 55.59 | 71.62 | 81.82 | 87.32 | 90.84 | |
| 0.01 | −9.84 | 55.35 | 69.21 | 81.36 | 86.5 | 90.56 | |
| 0.00 | 0 | 61.27 | 72.83 | 83.41 | 88.63 | 92.06 | |

| [DRUG] | | | | | | | STANDARD DEVIATION (%) |
|---|---|---|---|---|---|---|---|
| TAF | | | | | | | Horizontal axis: |
| nM | 0.00 | 0.04 | 0.11 | 0.33 | 1.00 | 3.00 | (V) (μg/mL) |
| 1.00 | 6.16 | 0.31 | 1.09 | 0.97 | 0.72 | 0.2 | |
| 0.33 | 8.67 | 0.71 | 1.05 | 1.09 | 0.4 | 0.28 | |
| 0.11 | 6.77 | 0.98 | 0.88 | 0.74 | 0.38 | 0.28 | |
| 0.04 | 3.94 | 2.8 | 0.52 | 1.17 | 0.58 | 0.3 | |
| 0.01 | 3.16 | 3.44 | 0.7 | 1.39 | 0.69 | 0.47 | |
| 0.00 | 9.05 | 7.13 | 2.25 | 1.79 | 0.72 | 0.29 | |

| [DRUG] | | | | | | | ADDITIVE % INHIBITION |
|---|---|---|---|---|---|---|---|
| TAF | | | | | | | Horizontal axis: |
| nM | 0.00 | 0.04 | 0.11 | 0.33 | 1.00 | 3.00 | (V) (μg/mL) |
| 1.00 | 29.9 | 72.85 | 80.95 | 88.37 | 92.03 | 94.43 | |
| 0.33 | 12.91 | 66.27 | 76.34 | 85.55 | 90.1 | 93.09 | |
| 0.11 | −4.19 | 59.65 | 71.69 | 82.71 | 88.15 | 91.73 | |
| 0.04 | −2.84 | 60.17 | 72.06 | 82.94 | 88.31 | 91.83 | |
| 0.01 | −9.84 | 57.46 | 70.16 | 81.78 | 87.51 | 91.28 | |
| 0.00 | 0 | 61.27 | 72.83 | 83.41 | 88.63 | 92.06 | |

| [DRUG] | | | | | | | SYNERGY PLOT (99.9%) |
|---|---|---|---|---|---|---|---|
| TAF | | | | | | | Bonferroni Adj. |
| nM | 0.00 | 0.04 | 0.11 | 0.33 | 1.00 | 3.00 | 98% |
| 1.00 | 0 | −1.02979 | 0 | 0 | 0 | −0.1218 | SYNERGY 0 |
| 0.33 | 0 | 0 | 0 | 0 | −0.1536 | −0.31852 | log volume 0 |
| 0.11 | 0 | 0 | 0 | 0 | 0 | −0.11852 | |
| 0.04 | 0 | 0 | 0 | 0 | 0 | −0.0027 | ANTAGONISM −1.74 |
| 0.01 | 0 | 0 | 0 | 0 | 0 | 0 | log volume −0.40 |
| 0.00 | 0 | 0 | 0 | 0 | 0 | 0 | |

TABLE 16
Summary of results of in vitro combination studies of a Compound
of Formula (V) and TAF in PHH cell culture system:

| HBV | TAF | (V) | | Synergy | | Antagonism | |
|---|---|---|---|---|---|---|---|
| Assay | EC₅₀ | EC₅₀ | Synergy | Log | Antagonism | Log | |
| Endpoint | (nM)# | (μg/mL)# | Volume* | Volume* | Volume* | Volume* | Conclusion |
| HBV | 0.083 | <0.123 | 88.42, 2.46 | 20.13, 0.56 | −1.1, −2.33 | −0.25, −0.53 | Additive to |
| DNA | | | | | | | Moderate |
| | | | | | | | Synergy |
| HBsAg | 4.119 | <0.123 | 0, 0 | 0, 0 | −2.46, −1.74 | −0.56, −0.40 | Additive |

*at 99.9% confidence interval
#determined in an earlier separate experiment

### Examples 5-14

The following compounds are referenced in the Examples. Compounds 1 and 2 can be prepared using known procedures (see, e.g., WO 2018/085619 and WO 2018/172852).

| Compound Number or Name | Structure |
|---|---|
| Compound 1 | 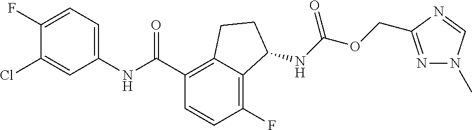 [SMILES: Cn1cnc(COC(=O)N[C@H]2CCc3c(C(=O)Nc4ccc(F)c(Cl)c4)ccc(F)c32)n1] |
| Compound 2 | 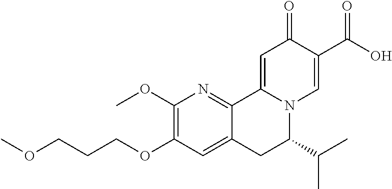 [SMILES: COCCCOc1cc2c(nc1OC)-c1cc(=O)c(C(=O)O)cn1[C@H](C(C)C)C2] |
| Entecavir (ETV) | 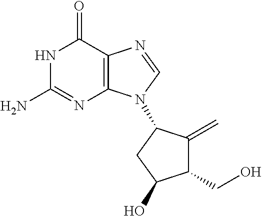 [SMILES: C=C1[C@H](CO)[C@@H](O)C[C@@H]1n1cnc2c(=O)[nH]c(N)nc21] |
| Tenofovir Disoproxil Fumarate (TDF) | 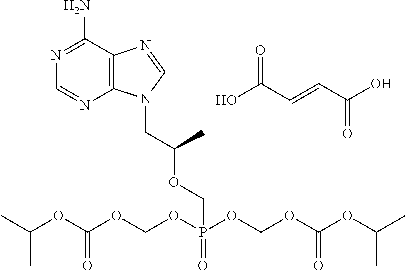 |
| Tenofovir Alafenamide (TAF) | 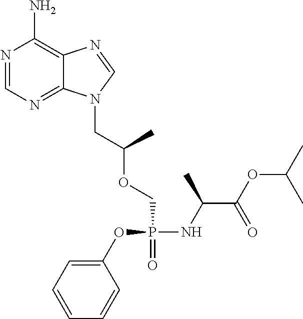 [SMILES: CC(C)OC(=O)[C@H](C)N[P@](=O)(CO[C@H](C)Cn1cnc2c(N)ncnc21)Oc1ccccc1] |

### Examples 5-8

### In Vitro Dual Combination Study Goal:

To determine whether two drug combinations of a small molecule inhibitor of HBV pgRNA encapsidation (Compound 1) with nucleos(t)ide analog inhibitor of HBV polymerase entecavir (ETV), tenofovir disoproxil fumarate (TDF) or tenofovir alafenamide (TAF) and SIRNA-NP, an siRNA formulation intended to facilitate potent knockdown of all viral mRNA transcripts and viral antigens, is additive, synergistic or antagonistic in vitro in HBV cell culture model systems.

### Composition of SIRNA-NP:

SIRNA-NP is a lipid nanoparticle formulation of a mixture of three siRNAs targeting the HBV genome. The following lipid nanoparticle (LNP) formulation was used to deliver the HBV siRNAs in the experiments reported herein. The values shown in the table are mole percentages. The abbreviation DSPC means distearoylphosphatidylcholine.

| PEG(20000)-C-DMA | Cationic lipid | Cholesterol | DSPC |
|---|---|---|---|
| 1.6 | 54.6 | 32.8 | 10.9 |

The cationic lipid had the following structure:

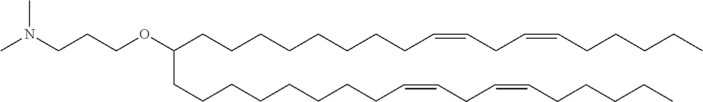 [SMILES: CCCCC/C=C\C/C=C\CCCCCCCCC(CCCCCCCC/C=C\C/C=C\CCCCC)OCCCN(C)C]

The sequences of the three siRNAs are shown below.

| | Sense Sequence | Antisense Sequence |
|---|---|---|
| Name | (5′-3′) | (5′-3′) |
| 3m | CCGUguGCACUuCGCuuCAUU | UGAAGCGAAGUgCACACgGUU |
| 6m | CuggCUCAGUUUACuAgUGUU | CACUAgUAAACUgAgCCAGUU |
| 12m | GCCgAuCCAUACugCGgAAUU | UUCCGCAgUAUGgAUCGgCUU |

lower case = 2′-O-methyl modification
Underline = unlocked nucleobase analogue (UNA) moiety

### In Vitro Dual Agent Combination in HepDE19 Cells Experimental Protocol:

In vitro dual agent combination studies were conducted using the method of Prichard and Shipman 1990 (Prichard M N, Shipman C, Jr. 1990. Antiviral Res 14:181-205). The HepDE19 cell culture system is a HepG2 (human hepatocarcinoma) derived cell line that supports HBV DNA replication and cccDNA formation under control the control of a CMV Tet-off promoter system (Guo et al. 2007. J Virol 81:12472-84). HepDE19 (50,000 cells/well) were plated in 96 well collagen-coated tissue-culture treated microtiter plates in DMEM/F12 medium supplemented with 10% fetal bovine serum+1% penicillin-streptomycin with tetracycline (1 μg/mL) and incubated in a humidified incubator at 37° C. and 5% CO₂ overnight. Next day, the cells were switched to fresh medium without tetracycline and incubated for 4 hrs at 37° C. and 5% CO₂. The cells were switched to fresh medium and treated with inhibitor A and inhibitor B, at concentration range spanning their respective EC₅₀ values. The inhibitors were either diluted in 100% DMSO (Compound 1, ETV, TDF and TAF) or growth medium (SIRNA-NP) and the final DMSO concentration in the assay was <0.5%. The two inhibitors were tested both singly as well as in combinations in a checkerboard fashion such that each concentration of inhibitor A was combined with each concentration of inhibitor B to determine their combination effects on inhibition of rcDNA production. There were four replicates of each concentration combination in each experiment. The plates were incubated for 7 days in a humidified incubator at 37° C. and 5% CO₂. The level of rcDNA present in the wells was measured using a Quantigene 2.0 bDNA assay kit (Affymetrix, Santa Clara, Calif.) with HBV specific custom probe set (genotype D ayw) and according to the manufacturer's instructions and read using a luminescence plate reader and the relative luminescence units (RLU) data generated from each well was calculated as % inhibition of the untreated control wells and analyzed using the MacSynergy II program to determine whether the combinations were synergistic, additive or antagonistic using the interpretive guidelines established by Prichard and Shipman (Prichard M N, Shipman C, Jr. 1990. Antiviral Res 14:181-205) as follows: synergy volumes <25 μM²% (log volume <2) at 99% CI (55% Bonferroni adjusted)=probably insignificant; 25-50 μM²% (log volume >2 and <5) at 99% CI (55% Bonferroni adjusted)=minor but significant; 50-100 μM²% (log volume >5 and <9) at 99% CI (55% Bonferroni adjusted)=moderate, may be important in vivo; over 100 μM²% (log volume >9) at 99% CI (55% Bonferroni adjusted)=strong synergy, probably important in vivo; volumes approaching 1000 μM²% (log volume >90)=unusually high, check data. Each experiment was repeated at least three times and the averages and standard deviations of individual determinations was calculated to derive the conclusion. Concurrently, in each experiment, the effect of inhibitor combinations on cell viability was assessed using replicate plates in triplicates that were used to determine the ATP content as a measure of cell viability using the Cell-Titer Glo reagent (Promega, Madison, Wis.) as per the manufacturer's instructions.

### Results and Conclusion:

### Example 5: In Vitro Dual Combination of Compound 1 and Entecavir (ETV) in HepDE19 Cells

Compound 1 (concentration range of 1.25 μM to 0.005 μM in a 2-fold dilution series and 9-point titration or a concentration range of 0.6 μM to 0.007 μM in a 3-fold dilution series and 5-point titration) was tested in combination with ETV (concentration range of 0.025 μM to 0.0003 μM in a 3-fold dilution series and 5-point titration or a concentration range of 0.050 μM to 0.0002 μM in a 2-fold dilution series and 9-point titration range, respectively). The average % inhibition in the amount of rcDNA and standard deviations of at least 3 replicates observed either with compound 1 or ETV treatment alone or in combination from each of 3 independent experiments is shown in Tables 1A-1C. The average EC₅₀ values of compound 1 and ETV are shown in Table 5. Whether the combination was additive, synergistic or antagonistic was determined based on the average synergy and antagonism volumes. When the observed values of two inhibitor combination were compared to what is expected from additive interaction for the above concentration range (at 99% confidence interval with 55% Bonferroni adjustment), the combinations were found to be additive (Table 5) as per MacSynergy II analysis and using the interpretive criteria described above by Prichard and Shipman (Prichard M N, Shipman C, Jr. 1990. Antiviral Res 14:181-205).

### Example 6: In Vitro Dual Combination of Compound 1 and Tenofovir Disoproxil Fumarate (TDF) in HepDE19 Cells

Compound 1 (concentration range of 1.25 μM to 0.005 μM in a 2-fold dilution series and 9-point titration or a concentration range of 0.6 μM to 0.007 μM in a 3-fold dilution series and 5-point titration) was tested in combination with TDF (concentration range of 0.750 μM to 0.009 μM in a 3-fold dilution series and 5-point titration or a concentration range of 2.5 μM to 0.01 μM in a 2-fold dilution series and 9-point titration range, respectively). The average % inhibition in the amount of rcDNA and standard deviations of 4 replicates observed either with compound 1 or TDF treatment alone or in combination from each of 3 independent experiments is shown in Tables 2A-2C. The average EC₅₀ values of compound 1 and TDF are shown in Table 5. Whether the combination was additive, synergistic or antagonistic was determined based on the average synergy and antagonism volumes. When the observed values of two inhibitor combination were compared to what is expected from additive interaction for the above concentration range (at 99% confidence interval with 55% Bonferroni adjustment), the combinations were found to be additive (Table 5) as per MacSynergy II analysis and using the interpretive criteria described above by Prichard and Shipman (Prichard M N, Shipman C, Jr. 1990. Antiviral Res 14:181-205).

### Example 7: In Vitro Dual Combination of Compound 1 and Tenofovir Alafenamide (TAF) in HepDE19 Cells

Compound 1 (concentration range of 1.25 μM to 0.005 μM in a 2-fold dilution series and 9-point titration or a concentration range of 0.6 μM to 0.007 μM in a 3-fold dilution series and 5-point titration) was tested in combination with TAF (concentration range of 0.18 μM to 0.002 μM in a 3-fold dilution series and 5-point titration or a concentration range of 0.32 μM to 0.001 μM in a 2-fold dilution series and 9-point titration range, respectively). The average % inhibition in the amount of rcDNA and standard deviations of at least 3 replicates observed either with compound 1 or TAF treatment alone or in combination from each of 4 independent experiments is shown in Tables 3A-3D. The average EC₅₀ values of compound 1 and TAF are shown in Table 5. Whether the combination was additive, synergistic or antagonistic was determined based on the average synergy and antagonism volumes. When the observed values of two inhibitor combination were compared to what is expected from additive interaction for the above concentration range (at 99% confidence interval with 55% Bonferroni adjustment), the combinations were found to be moderately synergistic (Table 5) as per MacSynergy II analysis and using the interpretive criteria described above by Prichard and Shipman (Prichard M N, Shipman C, Jr. 1990. Antiviral Res 14:181-205).

### Example 8: In Vitro Dual Combination of Compound 1 and SIRNA-NP in HepDE19 Cells

Compound 1 (concentration range of 1.25 μM to 0.005 μM in a 2-fold dilution series and 9-point titration or a concentration range of 0.6 μM to 0.007 μM in a 3-fold dilution series and 5-point titration) was tested in combination with SIRNA-NP (concentration range of 0.009m/mL to 0.0001 μg/mL in a 3-fold dilution series and 5-point titration or a concentration range of 0.016m/mL to 0.00006 μM in a 2-fold dilution series and 9-point titration range, respectively). The average % inhibition in the amount of rcDNA and standard deviations of 4 replicates observed either with compound 1 or SIRNA-NP treatment alone or in combination from each of 4 independent experiments is shown in Tables 4A-4C. The average EC₅₀ values of compound 1 and SIRNA-NP are shown in Table 5. Whether the combination was additive, synergistic or antagonistic was determined based on the average synergy and antagonism volumes. When the observed values of two inhibitor combination were compared to what is expected from additive interaction for the above concentration range (at 99% confidence interval with 55% Bonferroni adjustment), the combinations were found to be additive (Table 5) as per MacSynergy II analysis and using the interpretive criteria described above by Prichard and Shipman (Prichard M N, Shipman C, Jr. 1990. Antiviral Res 14:181-205).

TABLE 1A
In vitro Combination of Compound 1 and Entecavir (ETV) in HepDE19 cells: Expt 1

| [DRUG] | | | | | | | | | | | AVERAGE % | Cmpd |
|---|---|---|---|---|---|---|---|---|---|---|---|---|
| ETV μM | 0 | 0.005 | 0.010 | 0.020 | 0.039 | 0.078 | 0.156 | 0.313 | 0.625 | 1.25 | INHIBITION | 1 μM |
| 0.0250 | 95.3 | 93.23 | 95.22 | 95.1 | 96.29 | 97.83 | 98.68 | 98.7 | 99.14 | 99.46 | | |
| 0.0083 | 93.43 | 91.59 | 91.55 | 90.6 | 92.48 | 94.84 | 97.21 | 97.92 | 99.01 | 99.03 | | |
| 0.0028 | 81.82 | 79.85 | 78.37 | 81.68 | 85.02 | 92.8 | 96.46 | 97.58 | 98.47 | 99.2 | | |
| 0.0009 | 58.41 | 52.99 | 48.51 | 57.44 | 68.65 | 82.34 | 91.56 | 96.82 | 98.26 | 99.04 | | |
| 0.0003 | 24.31 | 6.49 | 10.93 | 24.97 | 33.63 | 67.6 | 90.5 | 93.28 | 97.22 | 98.7 | | |
| 0 | 0 | −5.55 | −2.14 | 20.8 | 28.33 | 62.79 | 82.02 | 91.07 | 95.23 | 98.94 | | |

| [DRUG] | | | | | | | | | | | STANDARD | Cmpd |
|---|---|---|---|---|---|---|---|---|---|---|---|---|
| ETV μM | 0 | 0.005 | 0.010 | 0.020 | 0.039 | 0.078 | 0.156 | 0.313 | 0.625 | 1.25 | DEVIATION (%) | 1 μM |
| 0.0250 | 1.59 | 1.36 | 1.68 | 1.28 | 1.48 | 0.16 | 0.33 | 0.73 | 0.31 | 0.26 | | |
| 0.0083 | 1.5 | 1.61 | 2.23 | 2.92 | 1.07 | 2.98 | 1.39 | 0.6 | 0.22 | 0.49 | | |
| 0.0028 | 5 | 2.29 | 3.48 | 11.35 | 10.39 | 3.7 | 1.04 | 1.2 | 0.37 | 0.14 | | |
| 0.0009 | 11 | 18.27 | 23.26 | 6.64 | 11.88 | 7.27 | 6.14 | 1.3 | 0.35 | 0.36 | | |
| 0.0003 | 13.61 | 28.98 | 21.64 | 23.16 | 22.56 | 10.96 | 4.85 | 1.22 | 0.81 | 0.71 | | |
| 0 | 0 | 19.12 | 12.08 | 38.59 | 27.54 | 8.09 | 6.15 | 2.2 | 1.93 | 0.32 | | |

| [DRUG] | | | | | | | | | | | ADDITIVE | Cmpd |
|---|---|---|---|---|---|---|---|---|---|---|---|---|
| ETV μM | 0 | 0.005 | 0.010 | 0.020 | 0.039 | 0.078 | 0.156 | 0.313 | 0.625 | 1.25 | INHIBITION | 1 μM |
| 0.0250 | 95.3 | 95.04 | 95.2 | 96.28 | 96.63 | 98.25 | 99.15 | 99.58 | 99.78 | 99.95 | | |
| 0.0083 | 93.43 | 93.07 | 93.29 | 94.8 | 95.29 | 97.56 | 98.82 | 99.41 | 99.69 | 99.93 | | |
| 0.0028 | 81.82 | 80.81 | 81.43 | 85.6 | 86.97 | 93.24 | 96.73 | 98.38 | 99.13 | 99.81 | | |
| 0.0009 | 58.41 | 56.1 | 57.52 | 67.06 | 70.19 | 84.52 | 92.52 | 96.29 | 98.02 | 99.56 | | |
| 0.0003 | 24.31 | 20.11 | 22.69 | 40.05 | 45.75 | 71.84 | 86.39 | 93.24 | 96.39 | 99.2 | | |
| 0 | 0 | −5.55 | −2.14 | 20.8 | 28.33 | 62.79 | 82.02 | 91.07 | 95.23 | 98.94 | | |

| [DRUG] | | | | | | | | | | | SYNERGY | Cmpd |
|---|---|---|---|---|---|---|---|---|---|---|---|---|
| ETV | | | | | | | | | | | PLOT (99%) | 1 μM |
| μM | 0 | 0.005 | 0.010 | 0.020 | 0.039 | 0.078 | 0.156 | 0.313 | 0.625 | 1.25 | Bonferroni Adj. | 55% |
| 0.0250 | 0 | 0 | 0 | 0 | 0 | −0.0088 | 0 | 0 | 0 | 0 | SYNERGY | 0 |
| 0.0083 | 0 | 0 | 0 | 0 | −0.0601 | 0 | 0 | 0 | −0.1146 | 0 | log volume | 0 |
| 0.0028 | 0 | 0 | 0 | 0 | 0 | 0 | 0 | 0 | 0 | −0.2502 | | |
| 0.0009 | 0 | 0 | 0 | 0 | 0 | 0 | 0 | 0 | 0 | 0 | ANTAGONISM | −0.43 |
| 0.0003 | 0 | 0 | 0 | 0 | 0 | 0 | 0 | 0 | 0 | 0 | log volume | −0.06 |
| 0 | 0 | 0 | 0 | 0 | 0 | 0 | 0 | 0 | 0 | 0 | | |

TABLE 1B
In vitro Combination of Compound 1 and Entecavir (ETV) in HepDE19 cells: Expt 2

| [DRUG] | | | | | | | | | | | AVERAGE % | ETV |
|---|---|---|---|---|---|---|---|---|---|---|---|---|
| Cmpd 1 μM | 0 | 0.0002 | 0.0004 | 0.0008 | 0.0016 | 0.0031 | 0.0063 | 0.0125 | 0.025 | 0.05 | INHIBITION | μM |
| 0.600 | 97.63 | 97.39 | 97.66 | 97.86 | 98.16 | 98.66 | 98.38 | 98.97 | 99.14 | 99.04 | | |
| 0.200 | 88.72 | 84.28 | 90.43 | 94.14 | 95.95 | 96.26 | 98.34 | 97.33 | 98.27 | 98.62 | | |
| 0.067 | 59.11 | 58.44 | 73.27 | 72.49 | 81.74 | 84.88 | 93.89 | 95.07 | 95.58 | 96 | | |
| 0.022 | 16.67 | 12.73 | 20.87 | 35.7 | 65.18 | 81.8 | 86.59 | 89.88 | 92.29 | 94.65 | | |
| 0.007 | 4.92 | −2.68 | 2.63 | 45.83 | 56.32 | 78.6 | 84.29 | 88.1 | 91.72 | 91.65 | | |
| 0 | 0 | −21.78 | 13.68 | 28.35 | 62 | 70.39 | 85.68 | 88.81 | 89.73 | 93.91 | | |

| [DRUG] | | | | | | | | | | | STANDARD | ETV |
|---|---|---|---|---|---|---|---|---|---|---|---|---|
| Cmpd 1 μM | 0 | 0.0002 | 0.0004 | 0.0008 | 0.0016 | 0.0031 | 0.0063 | 0.0125 | 0.025 | 0.05 | DEVIATION (%) | μM |
| 0.600 | 1.64 | 0.38 | 0.53 | 0.44 | 0.12 | 0.34 | 0.39 | 0.35 | 0.23 | 0.5 | | |
| 0.200 | 5.71 | 9.78 | 3.17 | 0.61 | 1.15 | 0.9 | 0.39 | 1.36 | 0.71 | 0.57 | | |
| 0.067 | 19.25 | 13.7 | 9.01 | 10.84 | 7.81 | 5.84 | 2.48 | 2.99 | 1.19 | 0.69 | | |
| 0.022 | 6.94 | 32.75 | 32.45 | 21.88 | 13.45 | 7.77 | 8.48 | 4.71 | 3.82 | 0.88 | | |
| 0.007 | 17.32 | 49.9 | 65.72 | 15.67 | 17.65 | 13.98 | 5.54 | 4.07 | 3.02 | 2.84 | | |
| 0 | 0 | 33.24 | 29.14 | 9.72 | 7.38 | 10.67 | 4.84 | 3.17 | 4.96 | 2.93 | | |

| [DRUG] | | | | | | | | | | | ADDITIVE | ETV |
|---|---|---|---|---|---|---|---|---|---|---|---|---|
| Cmpd 1 μM | 0 | 0.0002 | 0.0004 | 0.0008 | 0.0016 | 0.0031 | 0.0063 | 0.0125 | 0.025 | 0.05 | INHIBITION | μM |
| 0.600 | 97.63 | 97.11 | 97.95 | 98.3 | 99.1 | 99.3 | 99.66 | 99.73 | 99.76 | 99.86 | | |
| 0.200 | 88.72 | 86.26 | 90.26 | 91.92 | 95.71 | 96.66 | 98.38 | 98.74 | 98.84 | 99.31 | | |
| 0.067 | 59.11 | 50.2 | 64.7 | 70.7 | 84.46 | 87.89 | 94.14 | 95.42 | 95.8 | 97.51 | | |
| 0.022 | 16.67 | −1.48 | 28.07 | 40.29 | 68.33 | 75.33 | 88.07 | 90.68 | 91.44 | 94.93 | | |
| 0.007 | 4.92 | −15.79 | 17.93 | 31.88 | 63.87 | 71.85 | 86.38 | 89.36 | 90.24 | 94.21 | | |
| 0 | 0 | −21.78 | 13.68 | 28.35 | 62 | 70.39 | 85.68 | 88.81 | 89.73 | 93.91 | | |

| [DRUG] | | | | | | | | | | | SYNERGY | ETV |
|---|---|---|---|---|---|---|---|---|---|---|---|---|
| Cmpd 1 | | | | | | | | | | | PLOT (99%) | μM |
| μM | 0 | 0.0002 | 0.0004 | 0.0008 | 0.0016 | 0.0031 | 0.0063 | 0.0125 | 0.025 | 0.05 | Bonferroni Adj. | 55% |
| 0.600 | 0 | 0 | 0 | 0 | −0.6316 | 0 | −0.2777 | 0 | −0.0289 | 0 | SYNERGY | 0.65 |
| 0.200 | 0 | 0 | 0 | 0.6523 | 0 | 0 | 0 | 0 | 0 | 0 | log volume | 0.09 |
| 0.067 | 0 | 0 | 0 | 0 | 0 | 0 | 0 | 0 | 0 | 0 | | |
| 0.022 | 0 | 0 | 0 | 0 | 0 | 0 | 0 | 0 | 0 | 0 | ANTAGONISM | −0.94 |
| 0.007 | 0 | 0 | 0 | 0 | 0 | 0 | 0 | 0 | 0 | 0 | log volume | −0.14 |
| 0 | 0 | 0 | 0 | 0 | 0 | 0 | 0 | 0 | 0 | 0 | | |

TABLE 1C
In vitro Combination of Compound 1 and Entecavir (ETV) in HepDE19 cells: Expt 3

| [DRUG] | | | | | | | | | | | AVERAGE % | Compound 1 |
|---|---|---|---|---|---|---|---|---|---|---|---|---|
| ETV μM | 0 | 0.005 | 0.010 | 0.020 | 0.039 | 0.078 | 0.156 | 0.313 | 0.625 | 1.25 | INHIBITION | μM |
| 0.025 | 89.63 | 86.17 | 90.46 | 92.9 | 93.55 | 97.48 | 96.36 | 98.55 | 97.94 | 98.29 | | |
| 0.008 | 88.06 | 87.27 | 88.87 | 86.31 | 85.74 | 91.69 | 94.97 | 97.29 | 98.9 | 98.11 | | |
| 0.003 | 62.92 | 73.14 | 71.11 | 68.69 | 75.71 | 83.78 | 92.74 | 95.6 | 97.61 | 98.64 | | |
| 0.001 | 38.31 | 37.9 | 28.89 | 32.31 | 50.32 | 70.31 | 86.63 | 94.26 | 97.4 | 98.31 | | |
| 0.0003 | 28.28 | 1.47 | 17.18 | 7.94 | 31.79 | 64.22 | 88.81 | 94.32 | 96.91 | 98.39 | | |
| 0 | 0 | −5.5 | 6.89 | 14.2 | 43.73 | 49.62 | 80.63 | 92.09 | 96.72 | 98.11 | | |

| [DRUG] | | | | | | | | | | | STANDARD | Compound 1 |
|---|---|---|---|---|---|---|---|---|---|---|---|---|
| ETV μM | 0 | 0.005 | 0.010 | 0.020 | 0.039 | 0.078 | 0.156 | 0.313 | 0.625 | 1.25 | DEVIATION (%) | μM |
| 0.025 | 3.74 | 5.95 | 2.35 | 0.86 | 3.47 | 0.96 | 2.67 | 1.21 | 1.23 | 1.5 | | |
| 0.008 | 2.86 | 3.23 | 2.36 | 5.75 | 5.34 | 4.17 | 3.32 | 1.98 | 0.64 | 1.82 | | |
| 0.003 | 9.87 | 4.41 | 13.4 | 10.09 | 12.37 | 7.31 | 3.34 | 1.52 | 1.81 | 0.84 | | |
| 0.001 | 39.47 | 20.16 | 33.64 | 28.3 | 25.74 | 11.52 | 7.11 | 1.99 | 0.79 | 0.78 | | |
| 0.0003 | 23.78 | 39.73 | 26.79 | 43.09 | 25.26 | 9.22 | 5.36 | 1.87 | 1.18 | 0.82 | | |
| 0 | 0 | 40.64 | 36.53 | 35.96 | 27.21 | 21.71 | 7.09 | 3.65 | 0.72 | 1.14 | | |

| [DRUG] | | | | | | | | | | | ADDITIVE | Compound 1 |
|---|---|---|---|---|---|---|---|---|---|---|---|---|
| ETV μM | 0 | 0.005 | 0.010 | 0.020 | 0.039 | 0.078 | 0.156 | 0.313 | 0.625 | 1.25 | INHIBITION | μM |
| 0.025 | 89.63 | 89.06 | 90.34 | 91.1 | 94.16 | 94.78 | 97.99 | 99.18 | 99.66 | 99.8 | | |
| 0.008 | 88.06 | 87.4 | 88.88 | 89.76 | 93.28 | 93.98 | 97.69 | 99.06 | 99.61 | 99.77 | | |
| 0.003 | 62.92 | 60.88 | 65.47 | 68.19 | 79.14 | 81.32 | 92.82 | 97.07 | 98.78 | 99.3 | | |
| 0.001 | 38.31 | 34.92 | 42.56 | 47.07 | 65.29 | 68.92 | 88.05 | 95.12 | 97.98 | 98.83 | | |
| 0.0003 | 28.28 | 24.34 | 33.22 | 38.46 | 59.64 | 63.87 | 86.11 | 94.33 | 97.65 | 98.64 | | |
| 0 | 0 | −5.5 | 6.89 | 14.2 | 43.73 | 49.62 | 80.63 | 92.09 | 96.72 | 98.11 | | |

| [DRUG] | | | | | | | | | | | SYNERGY | Compound 1 |
|---|---|---|---|---|---|---|---|---|---|---|---|---|
| ETV | | | | | | | | | | | PLOT (99%) | μM |
| μM | 0 | 0.005 | 0.010 | 0.020 | 0.039 | 0.078 | 0.156 | 0.313 | 0.625 | 1.25 | Bonferroni Adj. | 55% |
| 0.025 | 0 | 0 | 0 | 0 | 0 | 0.2328 | 0 | 0 | 0 | 0 | SYNERGY | 1.16 |
| 0.008 | 0 | 0 | 0 | 0 | 0 | 0 | 0 | 0 | 0 | 0 | log volume | 0.17 |
| 0.003 | 0 | 0.9263 | 0 | 0 | 0 | 0 | 0 | 0 | 0 | 0 | | |
| 0.001 | 0 | 0 | 0 | 0 | 0 | 0 | 0 | 0 | 0 | 0 | ANTAGONISM | 0 |
| 0.0003 | 0 | 0 | 0 | 0 | 0 | 0 | 0 | 0 | 0 | 0 | log volume | 0 |
| 0 | 0 | 0 | 0 | 0 | 0 | 0 | 0 | 0 | 0 | 0 | | |

TABLE 2A
In vitro Combination of Compound 1 and Tenofovir Disoproxil Fumarate (TDF) in HepDE19 cells: Expt 1

| [DRUG] | | | | | | | | | | | AVERAGE % | Cmpd |
|---|---|---|---|---|---|---|---|---|---|---|---|---|
| TDF μM | 0 | 0.005 | 0.010 | 0.020 | 0.039 | 0.078 | 0.156 | 0.313 | 0.625 | 1.250 | INHIBITION | 1 μM |
| 0.750 | 98 | 97.72 | 97.33 | 98.32 | 98.04 | 99 | 98.97 | 98.89 | 99.25 | 99.27 | | |
| 0.250 | 87.41 | 89.41 | 88.92 | 84.51 | 89.58 | 93.41 | 94.89 | 96.88 | 98.63 | 99.11 | | |
| 0.083 | 45.88 | 50.32 | 47.1 | 51.88 | 63.88 | 68.19 | 86.85 | 94.51 | 96.45 | 98.38 | | |
| 0.028 | 11.67 | 13.96 | 1.47 | 4.8 | 18.32 | 58.22 | 80.2 | 92.73 | 95.35 | 97.7 | | |
| 0.009 | −0.25 | −20.72 | −19.83 | −11.95 | 22.2 | 39.17 | 77.16 | 90.33 | 95.66 | 97.27 | | |
| 0 | 0 | −44.87 | −40.7 | −21.55 | 0.64 | 36.71 | 68.16 | 87.84 | 94.32 | 97.35 | | |

| [DRUG] | | | | | | | | | | | STANDARD | Cmpd |
|---|---|---|---|---|---|---|---|---|---|---|---|---|
| TDF μM | 0 | 0.005 | 0.010 | 0.020 | 0.039 | 0.078 | 0.156 | 0.313 | 0.625 | 1.250 | DEVIATION (%) | 1 μM |
| 0.750 | 0.79 | 0.21 | 0.88 | 0.24 | 0.45 | 0.12 | 0.39 | 0.53 | 0.26 | 0.53 | | |
| 0.250 | 5.54 | 1.75 | 3.43 | 10.19 | 4.7 | 3.41 | 2.98 | 0.54 | 0.3 | 0.5 | | |
| 0.083 | 20.61 | 10.31 | 30.15 | 19.48 | 17.01 | 17.77 | 6.19 | 1.73 | 0.44 | 1.28 | | |
| 0.028 | 45.36 | 47.14 | 78.68 | 63.92 | 75.64 | 14.1 | 9.28 | 1.09 | 1.48 | 1.01 | | |
| 0.009 | 27.69 | 47.96 | 58.15 | 59.3 | 42.58 | 29.25 | 9.49 | 3.91 | 2.05 | 1.98 | | |
| 0 | 0 | 70.29 | 39.02 | 56.21 | 63.02 | 30.51 | 15.71 | 9.16 | 2.6 | 1.78 | | |

| [DRUG] | | | | | | | | | | | ADDITIVE | Cmpd |
|---|---|---|---|---|---|---|---|---|---|---|---|---|
| TDF μM | 0 | 0.005 | 0.010 | 0.020 | 0.039 | 0.078 | 0.156 | 0.313 | 0.625 | 1.250 | INHIBITION | 1 μM |
| 0.750 | 98 | 97.1 | 97.19 | 97.57 | 98.01 | 98.73 | 99.36 | 99.76 | 99.89 | 99.95 | | |
| 0.250 | 87.41 | 81.76 | 82.29 | 84.7 | 87.49 | 92.03 | 95.99 | 98.47 | 99.28 | 99.67 | | |
| 0.083 | 45.88 | 21.6 | 23.85 | 34.22 | 46.23 | 65.75 | 82.77 | 93.42 | 96.93 | 98.57 | | |
| 0.028 | 11.67 | −27.96 | −24.28 | −7.37 | 12.24 | 44.1 | 71.88 | 89.26 | 94.98 | 97.66 | | |
| 0.009 | −0.25 | −45.23 | −41.05 | −21.85 | 0.39 | 36.55 | 68.08 | 87.81 | 94.31 | 97.34 | | |
| 0 | 0 | −44.87 | −40.7 | −21.55 | 0.64 | 36.71 | 68.16 | 87.84 | 94.32 | 97.35 | | |

| [DRUG] | | | | | | | | | | | SYNERGY | Cmpd |
|---|---|---|---|---|---|---|---|---|---|---|---|---|
| TDF | | | | | | | | | | | PLOT (99%) | 1 μM |
| μM | 0 | 0.005 | 0.010 | 0.020 | 0.039 | 0.078 | 0.156 | 0.313 | 0.625 | 1.250 | Bonferroni Adj. | 55% |
| 0.750 | 0 | 0.0803 | 0 | 0.1332 | 0 | 0 | 0 | 0 | 0 | 0 | SYNERGY | 6.26 |
| 0.250 | 0 | 3.1525 | 0 | 0 | 0 | 0 | 0 | −0.2022 | 0 | 0 | log volume | 0.9 |
| 0.083 | 0 | 2.2233 | 0 | 0 | 0 | 0 | 0 | 0 | 0 | 0 | | |
| 0.028 | 0 | 0 | 0 | 0 | 0 | 0 | 0 | 0.6687 | 0 | 0 | ANTAGONISM | −0.2 |
| 0.009 | 0 | 0 | 0 | 0 | 0 | 0 | 0 | 0 | 0 | 0 | log volume | −0.03 |
| 0 | 0 | 0 | 0 | 0 | 0 | 0 | 0 | 0 | 0 | 0 | | |

TABLE 2B
In vitro Combination of Compound 1 and Tenofovir Disoproxil Fumarate (TDF) in HepDE19 cells: Expt 2

| [DRUG] | | | | | | | | | | | AVERAGE % | TDF |
|---|---|---|---|---|---|---|---|---|---|---|---|---|
| Compound 1 μM | 0 | 0.010 | 0.020 | 0.039 | 0.078 | 0.156 | 0.313 | 0.625 | 1.250 | 2.500 | INHIBITION | μM |
| 0.600 | 95.21 | 94.43 | 92.98 | 96.26 | 95.85 | 98.65 | 98.32 | 99.15 | 99.42 | 99.64 | | |
| 0.200 | 80.45 | 81.69 | 79.29 | 79.93 | 89.95 | 93.62 | 96.65 | 98.18 | 99.07 | 99.64 | | |
| 0.067 | 28.15 | 26.77 | 38.22 | 42.14 | 57 | 79.05 | 92.9 | 96.95 | 98.28 | 99.34 | | |
| 0.022 | 4.15 | −4.44 | 9.71 | 14.83 | 34.5 | 68.61 | 86.99 | 95.03 | 97.8 | 99.05 | | |
| 0.007 | −35.1 | −9.15 | 13.8 | 12.88 | 40.87 | 66.31 | 90.88 | 95.86 | 98.59 | 99.1 | | |
| 0 | 0 | 4.67 | −12.95 | 35.9 | 60.28 | 77.14 | 85.15 | 95.15 | 98.36 | 99.06 | | |

| [DRUG] | | | | | | | | | | | STANDARD | TDF |
|---|---|---|---|---|---|---|---|---|---|---|---|---|
| Compound 1 μM | 0 | 0.010 | 0.020 | 0.039 | 0.078 | 0.156 | 0.313 | 0.625 | 1.250 | 2.500 | DEVIATION (%) | μM |
| 0.600 | 0.68 | 0.78 | 2.86 | 0.29 | 2.1 | 0.45 | 0.69 | 0.3 | 0.38 | 0.21 | | |
| 0.200 | 3.34 | 8.15 | 3.82 | 5.92 | 3.23 | 1.34 | 0.99 | 0.84 | 0.22 | 0.1 | | |
| 0.067 | 17.9 | 21.72 | 5.41 | 21.15 | 16.96 | 2.18 | 1.9 | 1.03 | 0.6 | 0.26 | | |
| 0.022 | 15.8 | 37.7 | 14.33 | 18.28 | 16.65 | 11.17 | 3.08 | 2.31 | 0.82 | 0.11 | | |
| 0.007 | 25.42 | 23.25 | 6.6 | 16.57 | 13.93 | 10.62 | 4.95 | 0.7 | 0.63 | 0.18 | | |
| 0 | 0 | 24.61 | 53.06 | 20.23 | 16.3 | 3.03 | 6.61 | 1.92 | 0.18 | 0.36 | | |

| [DRUG] | | | | | | | | | | | ADDITIVE | TDF |
|---|---|---|---|---|---|---|---|---|---|---|---|---|
| Compound 1 μM | 0 | 0.010 | 0.020 | 0.039 | 0.078 | 0.156 | 0.313 | 0.625 | 1.250 | 2.500 | INHIBITION | μM |
| 0.600 | 95.21 | 95.43 | 94.59 | 96.93 | 98.1 | 98.91 | 99.29 | 99.77 | 99.92 | 99.95 | | |
| 0.200 | 80.45 | 81.36 | 77.92 | 87.47 | 92.23 | 95.53 | 97.1 | 99.05 | 99.68 | 99.82 | | |
| 0.067 | 28.15 | 31.51 | 18.85 | 53.94 | 71.46 | 83.58 | 89.33 | 96.52 | 98.82 | 99.32 | | |
| 0.022 | 4.15 | 8.63 | −8.26 | 38.56 | 61.93 | 78.09 | 85.77 | 95.35 | 98.43 | 99.1 | | |
| 0.007 | −35.1 | −28.79 | −52.6 | 13.4 | 46.34 | 69.12 | 79.94 | 93.45 | 97.78 | 98.73 | | |
| 0 | 0 | 4.67 | −12.95 | 35.9 | 60.28 | 77.14 | 85.15 | 95.15 | 98.36 | 99.06 | | |

| [DRUG] | | | | | | | | | | | SYNERGY | TDF |
|---|---|---|---|---|---|---|---|---|---|---|---|---|
| Compound | | | | | | | | | | | PLOT (99%) | μM |
| 1 μM | 0 | 0.010 | 0.020 | 0.039 | 0.078 | 0.156 | 0.313 | 0.625 | 1.250 | 2.500 | Bonferroni Adj. | 55% |
| 0.600 | 0 | 0 | 0 | 0 | 0 | 0 | 0 | 0 | 0 | 0 | SYNERGY | 55.52 |
| 0.200 | 0 | 0 | 0 | 0 | 0 | 0 | 0 | 0 | −0.0446 | 0 | log volume | 7.97 |
| 0.067 | 0 | 0 | 5.4663 | 0 | 0 | 0 | 0 | 0 | 0 | 0 | | |
| 0.022 | 0 | 0 | 0 | 0 | 0 | 0 | 0 | 0 | 0 | 0 | ANTAGONISM | −0.04 |
| 0.007 | 0 | 0 | 49.438 | 0 | 0 | 0 | 0 | 0.611 | 0 | 0 | log volume | −0.01 |
| 0 | 0 | 0 | 0 | 0 | 0 | 0 | 0 | 0 | 0 | 0 | | |

TABLE 2C
In vitro Combination of Compound 1 and Tenofovir Disoproxil Fumarate (TDF) in HepDE19 cells: Expt 3

| [DRUG] | | | | | | | | | | | AVERAGE % | Cmpd |
|---|---|---|---|---|---|---|---|---|---|---|---|---|
| TDF μM | 0 | 0.005 | 0.010 | 0.020 | 0.039 | 0.078 | 0.156 | 0.313 | 0.625 | 1.250 | INHIBITION | 1 μM |
| 0.750 | 96.97 | 96.08 | 97.02 | 98 | 97.01 | 98.59 | 98.4 | 98.87 | 99.32 | 99.48 | | |
| 0.250 | 92.8 | 92.35 | 90.42 | 89.87 | 90.68 | 94.43 | 97.45 | 97.73 | 98.62 | 99.07 | | |
| 0.083 | 76.04 | 67.79 | 65.36 | 65.11 | 72.28 | 83.73 | 93.47 | 96.64 | 98.04 | 99.21 | | |
| 0.028 | 34.29 | 49.42 | 39.15 | 44.58 | 49.66 | 69.04 | 85.7 | 94.15 | 97.04 | 98.45 | | |
| 0.009 | 32.54 | 17.53 | 29.07 | 47.41 | 54.22 | 66.52 | 85.81 | 93.85 | 97.42 | 98.68 | | |
| 0 | 0 | 18.78 | 27.29 | 44.53 | 66.81 | 68.11 | 83.63 | 94.66 | 97.86 | 98.8 | | |

| [DRUG] | | | | | | | | | | | STANDARD | Cmpd |
|---|---|---|---|---|---|---|---|---|---|---|---|---|
| TDF μM | 0 | 0.005 | 0.010 | 0.020 | 0.039 | 0.078 | 0.156 | 0.313 | 0.625 | 1.250 | DEVIATION (%) | 1 μM |
| 0.750 | 1.6 | 0.76 | 1.48 | 0.71 | 2.46 | 1.14 | 0.44 | 0.26 | 0.38 | 0.29 | | |
| 0.250 | 1.91 | 1.83 | 3.96 | 5.31 | 2.34 | 1.99 | 0.63 | 0.57 | 0.6 | 0.31 | | |
| 0.083 | 10.19 | 5.02 | 7.15 | 2.33 | 7.34 | 4.31 | 1.78 | 1 | 1.08 | 0.58 | | |
| 0.028 | 9.26 | 22.66 | 6.01 | 3.83 | 14.84 | 9.36 | 4.81 | 1.3 | 0.17 | 0.28 | | |
| 0.009 | 7.73 | 3.68 | 13.87 | 25.67 | 15.82 | 6.86 | 2.93 | 2.72 | 0.63 | 0.43 | | |
| 0 | 0 | 19.02 | 16.21 | 17.66 | 13.6 | 16.98 | 5.29 | 1.21 | 0.85 | 0.32 | | |

| [DRUG] | | | | | | | | | | | ADDITIVE | Cmpd |
|---|---|---|---|---|---|---|---|---|---|---|---|---|
| TDF μM | 0 | 0.005 | 0.010 | 0.020 | 0.039 | 0.078 | 0.156 | 0.313 | 0.625 | 1.250 | INHIBITION | 1 μM |
| 0.750 | 96.97 | 97.54 | 97.8 | 98.32 | 98.99 | 99.03 | 99.5 | 99.84 | 99.94 | 99.96 | | |
| 0.250 | 92.8 | 94.15 | 94.76 | 96.01 | 97.61 | 97.7 | 98.82 | 99.62 | 99.85 | 99.91 | | |
| 0.083 | 76.04 | 80.54 | 82.58 | 86.71 | 92.05 | 92.36 | 96.08 | 98.72 | 99.49 | 99.71 | | |
| 0.028 | 34.29 | 46.63 | 52.22 | 63.55 | 78.19 | 79.05 | 89.24 | 96.49 | 98.59 | 99.21 | | |
| 0.009 | 32.54 | 45.21 | 50.95 | 62.58 | 77.61 | 78.49 | 88.96 | 96.4 | 98.56 | 99.19 | | |
| 0 | 0 | 18.78 | 27.29 | 44.53 | 66.81 | 68.11 | 83.63 | 94.66 | 97.86 | 98.8 | | |

| [DRUG] | | | | | | | | | | | SYNERGY | Cmpd |
|---|---|---|---|---|---|---|---|---|---|---|---|---|
| TDF | | | | | | | | | | | PLOT (99%) | 1 μM |
| μM | 0 | 0.005 | 0.010 | 0.020 | 0.039 | 0.078 | 0.156 | 0.313 | 0.625 | 1.250 | Bonferroni Adj. | 55% |
| 0.750 | 0 | 0 | 0 | 0 | 0 | 0 | 0 | −0.3018 | 0 | 0 | SYNERGY | 0 |
| 0.250 | 0 | 0 | 0 | 0 | −0.9162 | 0 | 0 | −0.4251 | 0 | −0.0433 | log volume | 0 |
| 0.083 | 0 | 0 | 0 | −15.6119 | −0.9062 | 0 | 0 | 0 | 0 | 0 | | |
| 0.028 | 0 | 0 | 0 | −9.1269 | 0 | 0 | 0 | 0 | −1.1131 | −0.0404 | ANTAGONISM | −46.71 |
| 0.009 | 0 | −18.2224 | 0 | 0 | 0 | 0 | 0 | 0 | 0 | 0 | log volume | −6.71 |
| 0 | 0 | 0 | 0 | 0 | 0 | 0 | 0 | 0 | 0 | 0 | | |

TABLE 3A
In vitro Combination of Compound 1 and Tenofovir Alafenamide (TAF) in HepDE19 cells: Expt 1

| [DRUG] | | | | | | | | | | | AVERAGE % | Compound |
|---|---|---|---|---|---|---|---|---|---|---|---|---|
| TAF μM | 0 | 0.005 | 0.010 | 0.020 | 0.039 | 0.078 | 0.156 | 0.313 | 0.625 | 1.250 | INHIBITION | 1 μM |
| 0.180 | 88.31 | 88.48 | 91.08 | 90.09 | 92.57 | 93.16 | 94.65 | 96.78 | 97.64 | 99.43 | | |
| 0.060 | 67.04 | 59.25 | 57.38 | 61.22 | 75.68 | 88.61 | 91.6 | 95.89 | 95.96 | 98.79 | | |
| 0.020 | −1.17 | 20.25 | −18.8 | 11.62 | 0.7 | 57.1 | 82.87 | 92.8 | 96.01 | 97.1 | | |
| 0.007 | −30.18 | −71.56 | −23.24 | −22.1 | −71.04 | 25.26 | 69.88 | 86.91 | 92.67 | 96.89 | | |
| 0.002 | −55.34 | −20.83 | −67.02 | −56.83 | −7.15 | 40.33 | 74.92 | 92.03 | 94.28 | 97.35 | | |
| 0 | 0 | −81.65 | −86.44 | −84.72 | −14.16 | 39.97 | 59.35 | 90.16 | 95.07 | 98.27 | | |

| [DRUG] | | | | | | | | | | | STANDARD | Compound |
|---|---|---|---|---|---|---|---|---|---|---|---|---|
| TAF μM | 0 | 0.005 | 0.010 | 0.020 | 0.039 | 0.078 | 0.156 | 0.313 | 0.625 | 1.250 | DEVIATION (%) | 1 μM |
| 0.180 | 7.86 | 6.23 | 7.74 | 5.69 | 4.74 | 6.61 | 3.2 | 3.36 | 2.5 | 0.4 | | |
| 0.060 | 19.85 | 14.39 | 13.61 | 20.76 | 15.15 | 9.8 | 7.47 | 3.18 | 3.53 | 1.07 | | |
| 0.020 | 39.69 | 45.01 | 62.02 | 78.56 | 45.39 | 26.98 | 12.31 | 5.06 | 3.09 | 2.28 | | |
| 0.007 | 59.34 | 81.29 | 65.38 | 75.65 | 83.39 | 30.65 | 15.4 | 7.2 | 4.09 | 2.14 | | |
| 0.002 | 72.64 | 46.39 | 93.68 | 110.68 | 68.72 | 43.9 | 15.05 | 6.4 | 5.11 | 1.65 | | |
| 0 | 0 | 97.16 | 134.61 | 64.93 | 70.43 | 38.18 | 16.03 | 7.03 | 3.15 | 1.54 | | |

| [DRUG] | | | | | | | | | | | ADDITIVE | Compound |
|---|---|---|---|---|---|---|---|---|---|---|---|---|
| TAF μM | 0 | 0.005 | 0.010 | 0.020 | 0.039 | 0.078 | 0.156 | 0.313 | 0.625 | 1.250 | INHIBITION | 1 μM |
| 0.180 | 88.31 | 78.77 | 78.21 | 78.41 | 86.65 | 92.98 | 95.25 | 98.85 | 99.42 | 99.8 | | |
| 0.060 | 67.04 | 40.13 | 38.55 | 39.12 | 62.37 | 80.21 | 86.6 | 96.76 | 98.38 | 99.43 | | |
| 0.020 | −1.17 | −83.78 | −88.62 | −86.88 | −15.5 | 39.27 | 58.87 | 90.04 | 95.01 | 98.25 | | |
| 0.007 | −30.18 | −136.47 | −142.71 | −140.47 | −48.61 | 21.85 | 47.08 | 87.19 | 93.58 | 97.75 | | |
| 0.002 | −55.34 | −182.18 | −189.62 | −186.94 | −77.34 | 6.75 | 36.85 | 84.71 | 92.34 | 97.31 | | |
| 0 | 0 | −81.65 | −86.44 | −84.72 | −14.16 | 39.97 | 59.35 | 90.16 | 95.07 | 98.27 | | |

| [DRUG] | | | | | | | | | | | SYNERGY | Compound |
|---|---|---|---|---|---|---|---|---|---|---|---|---|
| TAF | | | | | | | | | | | PLOT (99%) | 1 μM |
| μM | 0 | 0.005 | 0.010 | 0.020 | 0.039 | 0.078 | 0.156 | 0.313 | 0.625 | 1.250 | Bonferroni Adj. | 55% |
| 0.180 | 0 | 0 | 0 | 0 | 0 | 0 | 0 | 0 | 0 | 0 | SYNERGY | 42.13 |
| 0.060 | 0 | 0 | 0 | 0 | 0 | 0 | 0 | 0 | 0 | 0 | log volume | 6.05 |
| 0.020 | 0 | 0 | 0 | 0 | 0 | 0 | 0 | 0 | 0 | 0 | | |
| 0.007 | 0 | 0 | 0 | 0 | 0 | 0 | 0 | 0 | 0 | 0 | ANTAGONISM | 0 |
| 0.002 | 0 | 42.1277 | 0 | 0 | 0 | 0 | 0 | 0 | 0 | 0 | log volume | 0 |
| 0 | 0 | 0 | 0 | 0 | 0 | 0 | 0 | 0 | 0 | 0 | | |

TABLE 3B
In vitro Combination of Compound 1 and Tenofovir Alafenamide (TAF) in HepDE19 cells: Expt 2

| [DRUG] | | | | | | | | | | | AVERAGE % | Cmpd |
|---|---|---|---|---|---|---|---|---|---|---|---|---|
| TAF μM | 0 | 0.005 | 0.010 | 0.020 | 0.039 | 0.078 | 0.156 | 0.313 | 0.625 | 1.250 | INHIBITION | 1 μM |
| 0.180 | 96.95 | 96.17 | 96.41 | 96.22 | 96.82 | 97.89 | 98.94 | 97.69 | 98.51 | 98.71 | | |
| 0.060 | 89.43 | 88.07 | 91.41 | 89.33 | 91.24 | 94.58 | 97.83 | 97.84 | 98.05 | 98.59 | | |
| 0.020 | 65.48 | 67.61 | 65.56 | 71.26 | 75.22 | 87.19 | 93.61 | 95.56 | 98.43 | 98.35 | | |
| 0.007 | 24.94 | 35 | 36.27 | 27.59 | 54.18 | 69.31 | 89.94 | 95.72 | 97.26 | 97.25 | | |
| 0.002 | 9.84 | 31.55 | 10.66 | 31.09 | 42.67 | 64.55 | 88.91 | 94.51 | 96.5 | 98.42 | | |
| 0 | 0 | 7.51 | 26.27 | 40.79 | 39.81 | 69.99 | 87.38 | 94.52 | 97.31 | 98.38 | | |

| [DRUG] | | | | | | | | | | | STANDARD | Cmpd |
|---|---|---|---|---|---|---|---|---|---|---|---|---|
| TAF μM | 0 | 0.005 | 0.010 | 0.020 | 0.039 | 0.078 | 0.156 | 0.313 | 0.625 | 1.250 | DEVIATION (%) | 1 μM |
| 0.180 | 1.54 | 1.17 | 1.07 | 1.42 | 0.51 | 1.1 | 0.48 | 1.09 | 0.23 | 0.29 | | |
| 0.060 | 5.03 | 2.18 | 0.66 | 3.11 | 0.75 | 0.79 | 1.7 | 0.89 | 0.49 | 0.8 | | |
| 0.020 | 5.63 | 5.94 | 4.55 | 3.02 | 4.32 | 5.83 | 3.92 | 2.56 | 0.34 | 0.59 | | |
| 0.007 | 30.92 | 6.94 | 2.16 | 18.77 | 10.03 | 3.33 | 4.82 | 2.14 | 0.61 | 0.85 | | |
| 0.002 | 14.22 | 26.7 | 21.56 | 7.77 | 25.57 | 8.72 | 3.28 | 0.47 | 0.48 | 0.33 | | |
| 0 | 0 | 22.65 | 11.94 | 9.69 | 11.42 | 3.93 | 2.51 | 1.14 | 0.71 | 0.4 | | |

| [DRUG] | | | | | | | | | | | ADDITIVE | Cmpd |
|---|---|---|---|---|---|---|---|---|---|---|---|---|
| TAF μM | 0 | 0.005 | 0.010 | 0.020 | 0.039 | 0.078 | 0.156 | 0.313 | 0.625 | 1.250 | INHIBITION | 1 μM |
| 0.180 | 96.95 | 97.18 | 97.75 | 98.19 | 98.16 | 99.08 | 99.62 | 99.83 | 99.92 | 99.95 | | |
| 0.060 | 89.43 | 90.22 | 92.21 | 93.74 | 93.64 | 96.83 | 98.67 | 99.42 | 99.72 | 99.83 | | |
| 0.020 | 65.48 | 68.07 | 74.55 | 79.56 | 79.22 | 89.64 | 95.64 | 98.11 | 99.07 | 99.44 | | |
| 0.007 | 24.94 | 30.58 | 44.66 | 55.56 | 54.82 | 77.47 | 90.53 | 95.89 | 97.98 | 98.78 | | |
| 0.002 | 9.84 | 16.61 | 33.53 | 46.62 | 45.73 | 72.94 | 88.62 | 95.06 | 97.57 | 98.54 | | |
| 0 | 0 | 7.51 | 26.27 | 40.79 | 39.81 | 69.99 | 87.38 | 94.52 | 97.31 | 98.38 | | |

| [DRUG] | | | | | | | | | | | SYNERGY | Cmpd |
|---|---|---|---|---|---|---|---|---|---|---|---|---|
| TAF | | | | | | | | | | | PLOT (99%) | 1 μM |
| μM | 0 | 0.005 | 0.010 | 0.020 | 0.039 | 0.078 | 0.156 | 0.313 | 0.625 | 1.250 | Bonferroni Adj. | 55% |
| 0.180 | 0 | 0 | 0 | 0 | −0.0293 | 0 | 0 | 0 | −0.8189 | −0.4947 | SYNERGY | 0 |
| 0.060 | 0 | 0 | 0 | 0 | −0.4725 | −0.2197 | 0 | 0 | −0.4107 | 0 | log volume | 0 |
| 0.020 | 0 | 0 | 0 | −0.5386 | 0 | 0 | 0 | 0 | 0 | 0 | | |
| 0.007 | 0 | 0 | −2.8388 | 0 | 0 | 0 | 0 | 0 | 0 | 0 | ANTAGONISM | −5.82 |
| 0.002 | 0 | 0 | 0 | 0 | 0 | 0 | 0 | 0 | 0 | 0 | log volume | −0.84 |
| 0 | 0 | 0 | 0 | 0 | 0 | 0 | 0 | 0 | 0 | 0 | | |

TABLE 3C
In vitro Combination of Compound 1 and Tenofovir Alafenamide (TAF) in HepDE19 cells: Expt 3

| [DRUG] | | | | | | | | | | | AVERAGE % | TAF |
|---|---|---|---|---|---|---|---|---|---|---|---|---|
| Cmpd 1 μM | 0 | 0.001 | 0.003 | 0.005 | 0.010 | 0.020 | 0.040 | 0.080 | 0.160 | 0.320 | INHIBITION | μM |
| 0.600 | 94.6 | 95.83 | 95.03 | 91.67 | 95.26 | 96.75 | 94.47 | 97.26 | 97.8 | 97.51 | | |
| 0.200 | 85.51 | 82.82 | 83.9 | 82.83 | 76.84 | 79.14 | 84.84 | 92.49 | 97.59 | 96.66 | | |
| 0.067 | 51.83 | 12.3 | 39.28 | 1.4 | 13.38 | 20.94 | 39.4 | 70.19 | 92.99 | 93.21 | | |
| 0.022 | 2.42 | −4.95 | −40.91 | −74.13 | −62.3 | −10.42 | 15.78 | 50.24 | 92.73 | 95.73 | | |
| 0.007 | −51.73 | −41.14 | −60.63 | −109.57 | −67.21 | −54.35 | 2.97 | 57.57 | 87.24 | 95.43 | | |
| 0 | 0 | −133.77 | −49.2 | −42.3 | −48.39 | −34.32 | 36.96 | 57.07 | 88.02 | 94.83 | | |

| [DRUG] | | | | | | | | | | | STANDARD | TAF |
|---|---|---|---|---|---|---|---|---|---|---|---|---|
| Cmpd 1 μM | 0 | 0.001 | 0.003 | 0.005 | 0.010 | 0.020 | 0.040 | 0.080 | 0.160 | 0.320 | DEVIATION (%) | μM |
| 0.600 | 1.53 | 0.44 | 1.86 | 6.43 | 0.82 | 1.37 | 2.95 | 1.41 | 0.96 | 1.01 | | |
| 0.200 | 4.35 | 8.2 | 7.53 | 6.35 | 7.91 | 12.25 | 2.67 | 5.31 | 0.91 | 2.14 | | |
| 0.067 | 11.25 | 25.24 | 27.65 | 82.67 | 59.67 | 20.86 | 10.51 | 24.41 | 1.56 | 6.51 | | |
| 0.022 | 11.72 | 29.03 | 49.1 | 94.18 | 54.37 | 88.17 | 58.44 | 22.73 | 6.65 | 1.42 | | |
| 0.007 | 6.4 | 39.92 | 31.22 | 70.63 | 120.27 | 107.85 | 30.62 | 15.33 | 1.29 | 2.03 | | |
| 0 | 0 | 142.82 | 135.22 | 93.98 | 105.52 | 71.31 | 27.56 | 44.22 | 4.98 | 2.48 | | |

| [DRUG] | | | | | | | | | | | ADDITIVE | TAF |
|---|---|---|---|---|---|---|---|---|---|---|---|---|
| Cmpd 1 μM | 0 | 0.001 | 0.003 | 0.005 | 0.010 | 0.020 | 0.040 | 0.080 | 0.160 | 0.320 | INHIBITION | μM |
| 0.600 | 94.6 | 87.38 | 91.94 | 92.32 | 91.99 | 92.75 | 96.6 | 97.68 | 99.35 | 99.72 | | |
| 0.200 | 85.51 | 66.13 | 78.38 | 79.38 | 78.5 | 80.54 | 90.87 | 93.78 | 98.26 | 99.25 | | |
| 0.067 | 51.83 | −12.61 | 28.13 | 31.45 | 28.52 | 35.3 | 69.63 | 79.32 | 94.23 | 97.51 | | |
| 0.022 | 2.42 | −128.11 | −45.59 | −38.86 | −44.8 | −31.07 | 38.49 | 58.11 | 88.31 | 94.96 | | |
| 0.007 | −51.73 | −254.7 | −126.38 | −115.91 | −125.15 | −103.8 | 4.35 | 34.86 | 81.82 | 92.16 | | |
| 0 | 0 | −133.77 | −49.2 | −42.3 | −48.39 | −34.32 | 36.96 | 57.07 | 88.02 | 94.83 | | |

| [DRUG] | | | | | | | | | | | SYNERGY | TAF |
|---|---|---|---|---|---|---|---|---|---|---|---|---|
| Cmpd 1 | | | | | | | | | | | PLOT (99%) | μM |
| μM | 0 | 0.001 | 0.003 | 0.005 | 0.010 | 0.020 | 0.040 | 0.080 | 0.160 | 0.320 | Bonferroni Adj. | 55% |
| 0.600 | 0 | 7.3192 | 0 | 0 | 1.1626 | 0.4791 | 0 | 0 | 0 | 0 | SYNERGY | 170.58 |
| 0.200 | 0 | 0 | 0 | 0 | 0 | 0 | 0 | 0 | 0 | 0 | log volume | 24.5 |
| 0.067 | 0 | 0 | 0 | 0 | 0 | 0 | −3.2193 | 0 | 0 | 0 | | |
| 0.022 | 0 | 48.5529 | 0 | 0 | 0 | 0 | 0 | 0 | 0 | 0 | ANTAGONISM | −3.22 |
| 0.007 | 0 | 110.9656 | 0 | 0 | 0 | 0 | 0 | 0 | 2.1047 | 0 | log volume | −0.46 |
| 0 | 0 | 0 | 0 | 0 | 0 | 0 | 0 | 0 | 0 | 0 | | |

TABLE 3D
In vitro Combination of Compound 1 and Tenofovir Alafenamide (TAF) in HepDE19 cells: Expt 4

| [DRUG] | | | | | | | | | | | AVERAGE % | TAF |
|---|---|---|---|---|---|---|---|---|---|---|---|---|
| Cmpd 1 μM | 0 | 0.001 | 0.003 | 0.005 | 0.010 | 0.020 | 0.040 | 0.080 | 0.160 | 0.320 | INHIBITION | μM |
| 0.600 | 96.43 | 97.08 | 97.08 | 97.67 | 96.29 | 96.21 | 97.17 | 98.32 | 98.53 | 99.3 | | |
| 0.200 | 90.02 | 91.54 | 86.94 | 89 | 88.48 | 89.74 | 92.77 | 95.43 | 97.39 | 98.53 | | |
| 0.067 | 52.75 | 51.03 | 51.74 | 50.19 | 57.71 | 53.56 | 69.22 | 74.32 | 91.45 | 97.14 | | |
| 0.022 | 1.92 | −22.02 | −12.33 | −13.92 | −4.67 | 24.25 | 35.97 | 59.71 | 84.96 | 97.03 | | |
| 0.007 | 11.66 | 28.1 | −28.3 | −14.66 | 11.73 | 12.71 | 24.92 | 50.61 | 84.35 | 96.4 | | |
| 0 | 0 | 9.66 | −0.11 | −1.36 | 0.94 | 36.58 | 61.8 | 57.24 | 83.57 | 95.9 | | |

| [DRUG] | | | | | | | | | | | STANDARD | TAF |
|---|---|---|---|---|---|---|---|---|---|---|---|---|
| Cmpd 1 μM | 0 | 0.001 | 0.003 | 0.005 | 0.010 | 0.020 | 0.040 | 0.080 | 0.160 | 0.320 | DEVIATION (%) | μM |
| 0.600 | 1.82 | 0.73 | 1.26 | 1.33 | 1.29 | 1.63 | 1.47 | 0.69 | 0.68 | 0.41 | | |
| 0.200 | 3.24 | 4.4 | 5.97 | 1.82 | 4.92 | 4.91 | 2.74 | 1.76 | 1.09 | 0.29 | | |
| 0.067 | 19.7 | 19.89 | 22.15 | 28.14 | 20.33 | 11.57 | 5.6 | 13.32 | 3.69 | 1.2 | | |
| 0.022 | 42.8 | 53.67 | 45.32 | 71.37 | 32.61 | 35.43 | 22.94 | 14.27 | 7.27 | 0.63 | | |
| 0.007 | 32.72 | 31.68 | 40.97 | 39.01 | 63.69 | 44.58 | 33.81 | 14.87 | 6.34 | 0.93 | | |
| 0 | 0 | 34.68 | 32 | 30.43 | 35.61 | 36.04 | 6.56 | 17.25 | 4.28 | 2.19 | | |

| [DRUG] | | | | | | | | | | | ADDITIVE | TAF |
|---|---|---|---|---|---|---|---|---|---|---|---|---|
| Cmpd 1 μM | 0 | 0.001 | 0.003 | 0.005 | 0.010 | 0.020 | 0.040 | 0.080 | 0.160 | 0.320 | INHIBITION | μM |
| 0.600 | 96.43 | 96.77 | 96.43 | 96.38 | 96.46 | 97.74 | 98.64 | 98.47 | 99.41 | 99.85 | | |
| 0.200 | 90.02 | 90.98 | 90.01 | 89.88 | 90.11 | 93.67 | 96.19 | 95.73 | 98.36 | 99.59 | | |
| 0.067 | 52.75 | 57.31 | 52.7 | 52.11 | 53.19 | 70.03 | 81.95 | 79.8 | 92.24 | 98.06 | | |
| 0.022 | 1.92 | 11.39 | 1.81 | 0.59 | 2.84 | 37.8 | 62.53 | 58.06 | 83.89 | 95.98 | | |
| 0.007 | 11.66 | 20.19 | 11.56 | 10.46 | 12.49 | 43.97 | 66.25 | 62.23 | 85.49 | 96.38 | | |
| 0 | 0 | 9.66 | −0.11 | −1.36 | 0.94 | 36.58 | 61.8 | 57.24 | 83.57 | 95.9 | | |

| [DRUG] | | | | | | | | | | | SYNERGY | TAF |
|---|---|---|---|---|---|---|---|---|---|---|---|---|
| Cmpd 1 | | | | | | | | | | | PLOT (99%) | μM |
| μM | 0 | 0.001 | 0.003 | 0.005 | 0.010 | 0.020 | 0.040 | 0.080 | 0.160 | 0.320 | Bonferroni Adj. | 55% |
| 0.600 | 0 | 0 | 0 | 0 | 0 | 0 | 0 | 0 | 0 | 0 | SYNERGY | 0 |
| 0.200 | 0 | 0 | 0 | 0 | 0 | 0 | 0 | 0 | 0 | −0.3147 | log volume | 0 |
| 0.067 | 0 | 0 | 0 | 0 | 0 | 0 | 0 | 0 | 0 | 0 | | |
| 0.022 | 0 | 0 | 0 | 0 | 0 | 0 | 0 | 0 | 0 | 0 | ANTAGONISM | −0.31 |
| 0.007 | 0 | 0 | 0 | 0 | 0 | 0 | 0 | 0 | 0 | 0 | log volume | −0.04 |
| 0 | 0 | 0 | 0 | 0 | 0 | 0 | 0 | 0 | 0 | 0 | | |

TABLE 4A
In vitro Combination of Compound 1 and SIRNA-NP in HepDE19 cells: Expt 1

| [DRUG] | | | | | | | | | | | AVERAGE % | SIRNA-NP |
|---|---|---|---|---|---|---|---|---|---|---|---|---|
| Cmpd 1 μM | 0 | 0.00006 | 0.00013 | 0.00025 | 0.00050 | 0.001 | 0.002 | 0.004 | 0.008 | 0.016 | INHIBITION | μg/mL |
| 0.600 | 92.93 | 91 | 92.19 | 92.88 | 91.97 | 93 | 91.7 | 94.46 | 92.03 | 92.59 | | |
| 0.200 | 78.42 | 80.12 | 78.25 | 77 | 78.45 | 81.84 | 86.78 | 87.71 | 92.23 | 92.17 | | |
| 0.067 | 43.71 | 20.18 | −0.92 | 17.32 | 23.26 | 41.65 | 54.28 | 66.28 | 80.04 | 85.78 | | |
| 0.022 | −17.56 | −18.39 | −87.13 | −49.48 | −45.85 | −33.49 | 9.21 | 48.01 | 69.13 | 84.82 | | |
| 0.007 | −23.53 | −66.45 | −72.86 | −68.13 | −58.48 | −27.45 | 17.12 | 41.95 | 64.7 | 86.91 | | |
| 0 | 0 | −0.64 | −37.66 | 11.16 | −11.38 | 24.8 | 23.22 | 51.92 | 72.82 | 84.39 | | |

| [DRUG] | | | | | | | | | | | STANDARD | SIRNA-NP |
|---|---|---|---|---|---|---|---|---|---|---|---|---|
| Cmpd 1 μM | 0 | 0.00006 | 0.00013 | 0.00025 | 0.0005 | 0.001 | 0.002 | 0.004 | 0.008 | 0.016 | DEVIATION (%) | μg/mL |
| 0.600 | 1.41 | 7.41 | 6.2 | 2.29 | 2.95 | 3.86 | 3.17 | 3.92 | 3.89 | 4.5 | | |
| 0.200 | 16.55 | 13.11 | 20.13 | 11.29 | 11.33 | 12.92 | 6.27 | 6.7 | 5.12 | 7.09 | | |
| 0.067 | 13.09 | 45.93 | 74.02 | 55.51 | 51.76 | 53.97 | 27.22 | 21.81 | 10.25 | 6.59 | | |
| 0.022 | 77.1 | 40.58 | 112.41 | 68.35 | 112.1 | 112.15 | 65.57 | 23.25 | 14.3 | 10.11 | | |
| 0.007 | 25.29 | 91.67 | 101.58 | 107.03 | 102.62 | 118.37 | 32.77 | 26.35 | 19.36 | 3.75 | | |
| 0 | 0 | 20.41 | 86.62 | 24.13 | 65.49 | 13.71 | 48.83 | 26.72 | 4.42 | 9.21 | | |

| [DRUG] | | | | | | | | | | | ADDITIVE | SIRNA-NP |
|---|---|---|---|---|---|---|---|---|---|---|---|---|
| Cmpd 1 μM | 0 | 0.00006 | 0.00013 | 0.00025 | 0.0005 | 0.001 | 0.002 | 0.004 | 0.008 | 0.016 | INHIBITION | μg/mL |
| 0.600 | 92.93 | 92.88 | 90.27 | 93.72 | 92.13 | 94.68 | 94.57 | 96.6 | 98.08 | 98.9 | | |
| 0.200 | 78.42 | 78.28 | 70.29 | 80.83 | 75.96 | 83.77 | 83.43 | 89.62 | 94.13 | 96.63 | | |
| 0.067 | 43.71 | 43.35 | 22.51 | 49.99 | 37.3 | 57.67 | 56.78 | 72.94 | 84.7 | 91.21 | | |
| 0.022 | −17.56 | −18.31 | −61.83 | −4.44 | −30.94 | 11.59 | 9.74 | 43.48 | 68.05 | 81.65 | | |
| 0.007 | −23.53 | −24.32 | −70.05 | −9.74 | −37.59 | 7.11 | 5.15 | 40.61 | 66.42 | 80.72 | | |
| 0 | 0 | −0.64 | −37.66 | 11.16 | −11.38 | 24.8 | 23.22 | 51.92 | 72.82 | 84.39 | | |

| [DRUG] | | | | | | | | | | | SYNERGY | SIRNA-NP |
|---|---|---|---|---|---|---|---|---|---|---|---|---|
| Cmpd 1 | | | | | | | | | | | PLOT (99%) | μg/mL |
| μM | 0 | 0.00006 | 0.00013 | 0.00025 | 0.0005 | 0.001 | 0.002 | 0.004 | 0.008 | 0.016 | Bonferroni Adj. | 55% |
| 0.600 | 0 | 0 | 0 | 0 | 0 | 0 | 0 | 0 | 0 | 0 | SYNERGY | 0 |
| 0.200 | 0 | 0 | 0 | 0 | 0 | 0 | 0 | 0 | 0 | 0 | log volume | 0 |
| 0.067 | 0 | 0 | 0 | 0 | 0 | 0 | 0 | 0 | 0 | 0 | | |
| 0.022 | 0 | 0 | 0 | 0 | 0 | 0 | 0 | 0 | 0 | 0 | ANTAGONISM | 0 |
| 0.007 | 0 | 0 | 0 | 0 | 0 | 0 | 0 | 0 | 0 | 0 | log volume | 0 |
| 0 | 0 | 0 | 0 | 0 | 0 | 0 | 0 | 0 | 0 | 0 | | |

TABLE 4B
In vitro Combination of Compound 1 and SIRNA-NP in HepDE19 cells: Expt 2

| [DRUG] | | | | | | | | | | | | |
|---|---|---|---|---|---|---|---|---|---|---|---|---|
| SIRNA-NP | | | | | | | | | | | AVERAGE % | Cmpd |
| μg/mL | 0 | 0.005 | 0.010 | 0.020 | 0.039 | 0.078 | 0.156 | 0.313 | 0.625 | 1.250 | INHIBITION | 1 μM |
| 0.009 | 89.38 | 90.39 | 91.75 | 93.12 | 94.25 | 96.47 | 98.27 | 98.68 | 98.79 | 99.04 | | |
| 0.003 | 69.07 | 61.91 | 60.73 | 59.44 | 70.19 | 85.42 | 94.33 | 97.36 | 98.5 | 98.63 | | |
| 0.001 | 48.11 | 35.56 | 40.73 | 30.99 | 46.63 | 68.92 | 88.21 | 95.93 | 97.88 | 98.61 | | |
| 0.0003 | 11.79 | 1.73 | 1.27 | −4.64 | 15.32 | 59.17 | 85.77 | 94.24 | 97.08 | 98.3 | | |
| 0.0001 | 3.29 | −2.03 | 9.18 | −10.91 | 19.24 | 59.19 | 83.1 | 93.68 | 97.02 | 98.1 | | |
| 0 | 0 | 5.58 | −0.61 | 6.6 | 25.88 | 58.73 | 84.12 | 93.93 | 96.81 | 98.26 | | |

| [DRUG] | | | | | | | | | | | STANDARD | Cmpd |
|---|---|---|---|---|---|---|---|---|---|---|---|---|
| SIRNA-NP μg/mL | 0 | 0.005 | 0.010 | 0.020 | 0.039 | 0.078 | 0.156 | 0.3125 | 0.625 | 1.25 | DEVIATION (%) | 1 μM |
| 0.009 | 3.23 | 9.11 | 3.16 | 2.64 | 1.29 | 0.64 | 0.62 | 0.59 | 0.64 | 0.8 | | |
| 0.003 | 11.67 | 11.37 | 21.05 | 22.69 | 13.88 | 7.2 | 2.13 | 0.76 | 0.32 | 1 | | |
| 0.001 | 17.03 | 21.71 | 5.13 | 24.26 | 23.17 | 15.52 | 4.34 | 0.65 | 1.4 | 0.76 | | |
| 0.0003 | 19.38 | 31.87 | 31.47 | 8.99 | 34.83 | 13.12 | 4.99 | 1.54 | 0.95 | 0.61 | | |
| 0.0001 | 31.15 | 18.92 | 20.32 | 58.62 | 18.6 | 5.85 | 7.48 | 3.09 | 1.06 | 0.71 | | |
| 0 | 0 | 9.13 | 18.91 | 7.45 | 13.92 | 19.54 | 8.09 | 3.25 | 1.13 | 0.46 | | |

| [DRUG] | | | | | | | | | | | ADDITIVE | Cmpd |
|---|---|---|---|---|---|---|---|---|---|---|---|---|
| SIRNA-NP μg/mL | 0 | 0.005 | 0.010 | 0.020 | 0.039 | 0.078 | 0.156 | 0.3125 | 0.625 | 1.25 | INHIBITION | 1 μM |
| 0.009 | 89.38 | 89.97 | 89.32 | 90.08 | 92.13 | 95.62 | 98.31 | 99.36 | 99.66 | 99.82 | | |
| 0.003 | 69.07 | 70.8 | 68.88 | 71.11 | 77.07 | 87.24 | 95.09 | 98.12 | 99.01 | 99.46 | | |
| 0.001 | 48.11 | 51.01 | 47.79 | 51.53 | 61.54 | 78.58 | 91.76 | 96.85 | 98.34 | 99.1 | | |
| 0.0003 | 11.79 | 16.71 | 11.25 | 17.61 | 34.62 | 63.6 | 85.99 | 94.65 | 97.19 | 98.47 | | |
| 0.0001 | 3.29 | 8.69 | 2.7 | 9.67 | 28.32 | 60.09 | 84.64 | 94.13 | 96.91 | 98.32 | | |
| 0 | 0 | 5.58 | −0.61 | 6.6 | 25.88 | 58.73 | 84.12 | 93.93 | 96.81 | 98.26 | | |

| [DRUG] | | | | | | | | | | | SYNERGY | Cmpd |
|---|---|---|---|---|---|---|---|---|---|---|---|---|
| SIRNA-NP | | | | | | | | | | | PLOT (99%) | 1 μM |
| μg/mL | 0 | 0.005 | 0.010 | 0.020 | 0.039 | 0.078 | 0.156 | 0.3125 | 0.625 | 1.25 | Bonferroni Adj. | 55% |
| 0.009 | 0 | 0 | 0 | 0 | 0 | 0 | 0 | 0 | 0 | 0 | SYNERGY | 0 |
| 0.003 | 0 | 0 | 0 | 0 | 0 | 0 | 0 | 0 | 0 | 0 | log volume | 0 |
| 0.001 | 0 | 0 | 0 | 0 | 0 | 0 | 0 | 0 | 0 | 0 | | |
| 0.0003 | 0 | 0 | 0 | 0 | 0 | 0 | 0 | 0 | 0 | 0 | ANTAGONISM | 0 |
| 0.0001 | 0 | 0 | 0 | 0 | 0 | 0 | 0 | 0 | 0 | 0 | log volume | 0 |
| 0 | 0 | 0 | 0 | 0 | 0 | 0 | 0 | 0 | 0 | 0 | | |

TABLE 4C
In vitro Combination of Compound 1 and SIRNA-NP in HepDE19 cells: Expt 3

| [DRUG] | | | | | | | | | | | AVERAGE % | SIRNA-NP |
|---|---|---|---|---|---|---|---|---|---|---|---|---|
| Cmpd 1 μM | 0 | 0.00006 | 0.00013 | 0.00025 | 0.0005 | 0.001 | 0.002 | 0.004 | 0.008 | 0.016 | INHIBITION | μg/mL |
| 0.600 | 97.4 | 97.72 | 97.58 | 98.44 | 98.18 | 98.33 | 98.39 | 98.28 | 98.79 | 98.73 | | |
| 0.200 | 92.66 | 90.78 | 92.43 | 94.1 | 94.78 | 95.58 | 97 | 97.09 | 97.56 | 98.06 | | |
| 0.067 | 64.67 | 67.6 | 70.43 | 62.51 | 62.22 | 73.25 | 85.39 | 91.57 | 94.99 | 97.41 | | |
| 0.022 | 35.54 | 32 | 17.69 | 30.27 | 30.59 | 51.58 | 69.6 | 81.42 | 92.39 | 94.87 | | |
| 0.007 | 6.78 | 10.38 | 12.02 | 25.58 | 31.59 | 42.07 | 64.26 | 75.43 | 90.16 | 95.27 | | |
| 0 | 0 | 1.26 | 15.74 | 51.64 | 42.74 | 54.73 | 68.27 | 82.14 | 88.24 | 95.7 | | |

| [DRUG] | | | | | | | | | | | STANDARD | SIRNA-NP |
|---|---|---|---|---|---|---|---|---|---|---|---|---|
| Cmpd 1 μM | 0 | 0.00006 | 0.00013 | 0.00025 | 0.0005 | 0.001 | 0.002 | 0.004 | 0.008 | 0.016 | DEVIATION (%) | μg/mL |
| 0.600 | 0.49 | 0.52 | 0.65 | 0.82 | 0.57 | 0.68 | 0.98 | 0.71 | 0.14 | 0.93 | | |
| 0.200 | 2.32 | 4.58 | 2.32 | 2.73 | 1.38 | 1.54 | 0.62 | 0.96 | 0.76 | 0.87 | | |
| 0.067 | 6.35 | 10.12 | 10.92 | 11.21 | 19.11 | 9.03 | 2.42 | 1.32 | 0.76 | 0.95 | | |
| 0.022 | 10.46 | 16.35 | 35.27 | 10.71 | 11.18 | 7.92 | 6.31 | 6.25 | 0.5 | 1.16 | | |
| 0.007 | 26.57 | 7.04 | 11.71 | 5.35 | 2.78 | 14.04 | 4.35 | 7.75 | 1.78 | 1.16 | | |
| 0 | 0 | 19.79 | 13.46 | 27.78 | 5.28 | 2.43 | 5.42 | 5.79 | 1.93 | 0.28 | | |

| [DRUG] | | | | | | | | | | | ADDITIVE | SIRNA-NP |
|---|---|---|---|---|---|---|---|---|---|---|---|---|
| Cmpd 1 μM | 0 | 0.00006 | 0.00013 | 0.00025 | 0.0005 | 0.001 | 0.002 | 0.004 | 0.008 | 0.016 | INHIBITION | μg/mL |
| 0.600 | 97.4 | 97.43 | 97.81 | 98.74 | 98.51 | 98.82 | 99.18 | 99.54 | 99.69 | 99.89 | | |
| 0.200 | 92.66 | 92.75 | 93.82 | 96.45 | 95.8 | 96.68 | 97.67 | 98.69 | 99.14 | 99.68 | | |
| 0.067 | 64.67 | 65.12 | 70.23 | 82.91 | 79.77 | 84.01 | 88.79 | 93.69 | 95.85 | 98.48 | | |
| 0.022 | 35.54 | 36.35 | 45.69 | 68.83 | 63.09 | 70.82 | 79.55 | 88.49 | 92.42 | 97.23 | | |
| 0.007 | 6.78 | 7.95 | 21.45 | 54.92 | 46.62 | 57.8 | 70.42 | 83.35 | 89.04 | 95.99 | | |
| 0 | 0 | 1.26 | 15.74 | 51.64 | 42.74 | 54.73 | 68.27 | 82.14 | 88.24 | 95.7 | | |

| [DRUG] | | | | | | | | | | | SYNERGY | SIRNA-NP |
|---|---|---|---|---|---|---|---|---|---|---|---|---|
| Cmpd 1 | | | | | | | | | | | PLOT (99%) | μg/mL |
| μM | 0 | 0.00006 | 0.00013 | 0.00025 | 0.0005 | 0.001 | 0.002 | 0.004 | 0.008 | 0.016 | Bonferroni Adj. | 55% |
| 0.600 | 0 | 0 | 0 | 0 | 0 | 0 | 0 | 0 | −0.5402 | 0 | SYNERGY | 0 |
| 0.200 | 0 | 0 | 0 | 0 | 0 | 0 | 0 | 0 | 0 | 0 | log volume | 0 |
| 0.067 | 0 | 0 | 0 | 0 | 0 | 0 | 0 | 0 | 0 | 0 | | |
| 0.022 | 0 | 0 | 0 | −11.0353 | −3.7674 | 0 | 0 | 0 | 0 | 0 | ANTAGONISM | −38.82 |
| 0.007 | 0 | 0 | 0 | −15.5905 | −7.8854 | 0 | 0 | 0 | 0 | 0 | log volume | −5.58 |
| 0 | 0 | 0 | 0 | 0 | 0 | 0 | 0 | 0 | 0 | 0 | | |

TABLE 5
Summary of results of in vitro combination studies in HepDE19
cell culture system with rcDNA quantitation using bDNA assay:

| | | Avg. | Avg. | Avg. | Avg. | Avg. | Avg. | |
|---|---|---|---|---|---|---|---|---|
| | | Inhibitor A | Inhibitor B | Synergy | Synergy | Antagonism | Antagonism | |
| Inhibitor | Inhibitor | EC₅₀ | EC₅₀ | Volume | Log | Volume | Log | |
| A | B | (μM) | (μM or *μg/mL) | (μM² %) | Volume | (μM² %) | Volume | Conclusion |
| Cmpd 1 | ETV | 0.059 | 0.001 | 0.603 | 0.087 | −0.457 | −0.067 | Additive |
| (n = 3) | | (0.004) | (0.001) | (0.581) | (0.085) | (0.471) | (0.070) | |
| Cmpd 1 | TDF | 0.080 | 0.064 | 20.593 | 2.957 | −15.650 | −2.250 | Additive |
| (n = 3) | | (0.048) | (0.029) | (30.409) | (4.365) | (26.899) | (3.862) | |
| Cmpd 1 | TAF | 0.074 | 0.044 | 53.178 | 7.638 | −2.338 | −0.335 | Moderate |
| (n = 4) | | (0.036) | (0.023) | (80.749) | (11.598) | (2.737) | (0.396) | Synergy |
| Cmpd 1 | SIRNA-NP* | 0.064 | 0.002 | 0 | 0 | −12.940 | −1.860 | Additive |
| (n = 3) | | (0.025) | (0.002) | (0) | (0) | (22.413) | (3.222) | |

Notes:
SIRNA-NP EC₅₀ values expressed in μg/mL; Values in parenthesis are standard deviations of the mean. These values were determined at 99% confidence interval with 55% Bonferroni correction. The n value refers to the number of independent determinations.

### Examples 9-11

### In Vitro Triple Combination Study Goal:

To determine whether three drug combinations of a small molecule inhibitor of HBV pgRNA encapsidation (Compound 1) with an HBV RNA destabilizer (Compound 2) and a nucleos(t)ide analog inhibitor of HBV polymerase entecavir (ETV), tenofovir disoproxil fumarate (TDF) or tenofovir alafenamide (TAF), is additive, synergistic or antagonistic in vitro in a HBV cell culture model systems.

### In Vitro Triple Agent Combination in HepG 2.2.15 Cells: Experimental Protocol:

In vitro triple agent combination studies were conducted using the method of Prichard and Shipman 1990 (Prichard M N, Shipman C, Jr. 1990. Antiviral Res 14:181-205). The HepG 2.2.15 cell line was derived from HepG2 cells with constitutive expression of HBV (genotype D, serotype ayw) (Sells M A, Chen M L, Acs G. 1987. Proc Natl Acad Sci USA 84:1005-9). HepG 2.2.15 (10,000 cells/well) were plated in 96 well collagen-coated tissue-culture treated microtiter plates in RPMI 1640 medium supplemented with 10% fetal bovine serum+1% penicillin-streptomycin+200 mg G418/L and incubated in a humidified incubator at 37° C. and 5% CO₂ overnight. Next day, the cells were treated with Compound 1 and Compound 2, at concentration range spanning their respective EC₅₀ values. The inhibitors were diluted in 100% DMSO (Compound 1, Compound 2, ETV, TDF and TAF) and the final DMSO concentration in the assay was <0.5%. Triple combination studies were conducted in a checkerboard fashion such that each concentration of Compound 1 was combined with each concentration of Compound 2 in the presence of a fixed concentrations (including an arm with 0 concentration) of the third agent (ETV, TDF or TAF) to determine their combination effects on inhibition of rcDNA production in culture supernatant. There were four replicates of each concentration combination of Compound 1+Compound 2 for each single concentration of the third agent. The plates were incubated for 7 days in a humidified incubator at 37° C. and 5% CO₂. The level of rcDNA present in the culture supernatants was measured using a Quantigene 2.0 bDNA assay kit (Affymetrix, Santa Clara, Calif.) with HBV specific custom probe set (genotype D ayw) and according to the manufacturer's instructions and read using a luminescence plate reader and the relative luminescence units (RLU) data generated from each well was calculated as % inhibition of the untreated control wells and analyzed using the MacSynergy II program to determine whether the combinations were synergistic, additive or antagonistic using the interpretive guidelines established by Prichard and Shipman (Prichard M N, Shipman C, Jr. 1990. Antiviral Res 14:181-205) as follows: synergy volumes <25 μM²% (log volume <2) at 99% CI (55% Bonferroni adjusted)=probably insignificant; 25-50 μM²% (log volume >2 and <5) at 99% CI (55% Bonferroni adjusted)=minor but significant; 50-100 μM²% (log volume >5 and <9) at 99% CI (55% Bonferroni adjusted)=moderate, may be important in vivo; over 100 μM²% (log volume >9) at 99% CI (55% Bonferroni adjusted)=strong synergy, probably important in vivo; volumes approaching 1000 μM²% (log volume >90)=unusually high, check data. Concurrently, in each experiment, the effect of inhibitor combinations on cell viability was assessed in triplicates that were used to determine the ATP content as a measure of cell viability using the Cell-Titer Glo reagent (Promega, Madison, Wis.) as per the manufacturer's instructions.

### Results and Conclusion of In Vitro Triple Combination Studies:

### Example 9: In Vitro Triple Combination of Compound 1+Compound 2 and Entecavir (ETV) in HepG 2.2.15 Cells

Compound 1 (concentration range of 0.405 μM to 0.005 μM in a 3-fold dilution series and 5-point titration) was tested in combination with compound 2 (concentration range of 0.005 μM to 0.00002 μM in a 2-fold dilution series and 9-point titration) at different fixed concentrations of ETV (concentration range of 0.0003 μM to 0.0009 μM in a 3-fold dilution series including a 0 μM ETV concentration, a dual combination arm). The average % inhibition in the amount of rcDNA and standard deviations observed either with compound 1 or compound 2 alone or in triple combination with different concentrations of ETV is shown in Tables 6A-6E. Whether the combination was additive, synergistic or antagonistic was determined based on the average synergy and antagonism volumes. When the observed values of dual and triple inhibitor combination were compared to what is expected from additive interaction for the above concentration range (at 99% confidence interval with 55% Bonferroni adjustment), the combinations were found to be additive (Table 9) as per MacSynergy II analysis and using the interpretive criteria described above by Prichard and Shipman (Prichard M N, Shipman C, Jr. 1990. Antiviral Res 14:181-205).

### Example 10: In Vitro Triple Combination of Compound 1+Compound 2+Tenofovir Alafenamide (TAF) in HepG 2.2.15 Cells

Compound 1 (concentration range of 0.405 μM to 0.005 μM in a 3-fold dilution series and 5-point titration) was tested in combination with compound 2 (concentration range of 0.027 μM to 0.0001 μM in a 2-fold dilution series and 9-point titration) at different fixed concentrations of TAF (concentration range of 0.003 μM to 0.100 μM in a 3-fold dilution series including a 0 μM TAF concentration dual combination arm). The average % inhibition in the amount of rcDNA and standard deviations observed either with compound 1 or compound 2 alone or in triple combination with different concentrations of TAF is shown in Tables 7A-7E. Whether the combination was additive, synergistic or antagonistic was determined based on the average synergy and antagonism volumes. When the observed values of dual and triple inhibitor combination were compared to what is expected from additive interaction for the above concentration range (at 99% confidence interval with 55% Bonferroni adjustment), the combinations were found to be additive (Table 10) as per MacSynergy II analysis and using the interpretive criteria described above by Prichard and Shipman (Prichard M N, Shipman C, Jr. 1990. Antiviral Res 14:181-205).

### Example 11: In vitro triple combination of Compound 1+Compound 2+tenofovir disoproxil fumarate (TDF) in HepG 2.2.15 Cells

Compound 1 (concentration range of 0.405 μM to 0.005 μM in a 3-fold dilution series and 5-point titration) was tested in combination with compound 2 (concentration range of 0.027 μM to 0.0001 μM in a 2-fold dilution series and 9-point titration) at different fixed concentrations of TDF (concentration range of 0.010 μM to 0.100 μM in a 3-fold dilution series including a 0 μM TDF concentration dual combination arm). The average % inhibition in the amount of rcDNA and standard deviations observed either with compound 1 or compound 2 alone or in triple combination with different concentrations of TDF is shown in Tables 8A-8D. Whether the combination was additive, synergistic or antagonistic was determined based on the average synergy and antagonism volumes. When the observed values of dual and triple inhibitor combination were compared to what is expected from additive interaction for the above concentration range (at 99% confidence interval with 55% Bonferroni adjustment), the combinations were found to be additive (Table 11) as per MacSynergy II analysis and using the interpretive criteria described above by Prichard and Shipman (Prichard M N, Shipman C, Jr. 1990. Antiviral Res 14:181-205).

TABLE 6A
In vitro Combination of Compound 1 and Compound 2 in presence of ETV @ 0 μM (HepG 2.2.15 cell culture model):

| [DRUG] | | | | | | | | | | | AVERAGE % | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 92.67 | 93.51 | 93.13 | 93.88 | 94.37 | 94.12 | 94.66 | 94.53 | 94.77 | 94.23 | | |
| 0.135 | 81.13 | 82.56 | 86.57 | 85.33 | 89.24 | 91.27 | 92.34 | 93.03 | 93.1 | 93.47 | | |
| 0.045 | 42.33 | 59.54 | 67.84 | 75.66 | 79.77 | 84.49 | 87.71 | 88.76 | 90.67 | 91.26 | | |
| 0.015 | 4.33 | 38.51 | 52.51 | 65.12 | 75.54 | 80.77 | 86.34 | 88.04 | 90.04 | 89.21 | | |
| 0.005 | 3.45 | 36.76 | 47.21 | 61.86 | 75.01 | 77.94 | 83.91 | 86.63 | 88.51 | 89.12 | | |
| 0 | 0 | 39.44 | 47.6 | 67.99 | 74.24 | 77.97 | 83.78 | 86.78 | 88.65 | 88.04 | | |

| [DRUG] | | | | | | | | | | | STANDARD | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | DEVIATION (%) | 2 μM |
| 0.405 | 2.97 | 2.01 | 2.54 | 1.58 | 1.19 | 1.78 | 1.13 | 1.08 | 1.13 | 1.43 | | |
| 0.135 | 9.55 | 10.97 | 5.23 | 7.07 | 4.14 | 2.94 | 2.41 | 2.06 | 1.6 | 1.88 | | |
| 0.045 | 19.6 | 13.25 | 10 | 4.71 | 5.44 | 4.65 | 3.69 | 3.21 | 2.97 | 1.88 | | |
| 0.015 | 12.29 | 11.33 | 2.33 | 6.54 | 4.16 | 4.51 | 1.85 | 1.46 | 1.66 | 1.59 | | |
| 0.005 | 5.48 | 3.2 | 3.68 | 7.16 | 1.12 | 4.01 | 4.08 | 1.81 | 2.71 | 1.44 | | |
| 0 | 0 | 4.56 | 5.69 | 3 | 3.93 | 6.97 | 4.56 | 2.57 | 2.43 | 3.97 | | |

| [DRUG] | | | | | | | | | | | ADDITIVE | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 92.67 | 95.56 | 96.16 | 97.65 | 98.11 | 98.39 | 98.81 | 99.03 | 99.17 | 99.12 | | |
| 0.135 | 81.13 | 88.57 | 90.11 | 93.96 | 95.14 | 95.84 | 96.94 | 97.51 | 97.86 | 97.74 | | |
| 0.045 | 42.33 | 65.08 | 69.78 | 81.54 | 85.14 | 87.3 | 90.65 | 92.38 | 93.45 | 93.1 | | |
| 0.015 | 4.33 | 42.06 | 49.87 | 69.38 | 75.36 | 78.92 | 84.48 | 87.35 | 89.14 | 88.56 | | |
| 0.005 | 3.45 | 41.53 | 49.41 | 69.09 | 75.13 | 78.73 | 84.34 | 87.24 | 89.04 | 88.45 | | |
| 0 | 0 | 39.44 | 47.6 | 67.99 | 74.24 | 77.97 | 83.78 | 86.78 | 88.65 | 88.04 | | |

| [DRUG] | | | | | | | | | | | SYNERGY | Cmpd |
|---|---|---|---|---|---|---|---|---|---|---|---|---|
| Cmpd 1 | | | | | | | | | | | PLOT (99%) | 2 μM |
| μM | 0 | 0.0001 | 0.0002 | 0.0004 | 0.001 | 0.002 | 0.003 | 0.007 | 0.013 | 0.027 | Bonferroni Adj. | 55% |
| 0.405 | 0 | 0 | 0 | 0 | −0.6817 | 0 | −1.2459 | −1.7244 | −1.4959 | −1.2149 | SYNERGY | 0 |
| 0.135 | 0 | 0 | 0 | 0 | 0 | 0 | 0 | 0 | −0.648 | 0 | log volume | 0 |
| 0.045 | 0 | 0 | 0 | 0 | 0 | 0 | 0 | 0 | 0 | 0 | | |
| 0.015 | 0 | 0 | 0 | 0 | 0 | 0 | 0 | 0 | 0 | 0 | ANTAGONISM | −7.01 |
| 0.005 | 0 | 0 | 0 | 0 | 0 | 0 | 0 | 0 | 0 | 0 | log volume | −1.01 |
| 0 | 0 | 0 | 0 | 0 | 0 | 0 | 0 | 0 | 0 | 0 | | |

TABLE 6B
In vitro Combination of Compound 1 and Compound 2 in presence of
ETV @ 0.0003 μM (HepG 2.2.15 cell culture model)

| [DRUG] | | | | | | | | | | | AVERAGE % | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 90.85 | 90.98 | 90.87 | 91.82 | 91.85 | 91.73 | 89.63 | 82.46 | 87.11 | 91.6 | | |
| 0.135 | 76.77 | 84.08 | 85.1 | 86.5 | 87.96 | 88.79 | 87.81 | 88.12 | 89.51 | 90.56 | | |
| 0.045 | 37.72 | 60.57 | 65.91 | 75 | 78.4 | 74.98 | 80.95 | 86.71 | 88.44 | 88.12 | | |
| 0.015 | 15.85 | 40.13 | 50.78 | 65.36 | 73.39 | 79.24 | 82.79 | 85.47 | 85.8 | 86.67 | | |
| 0.005 | 6.13 | 40.37 | 45.37 | 60.54 | 71.37 | 77.51 | 81.44 | 84.23 | 85.66 | 86.6 | | |
| 0 | 0 | 31 | 51.33 | 62.94 | 73.38 | 77.97 | 81.52 | 84.36 | 85.67 | 86.38 | | |

| [DRUG] | | | | | | | | | | | STANDARD | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | DEVIATION (%) | 2 μM |
| 0.405 | 4.36 | 4.24 | 3.35 | 3.16 | 2.95 | 2.79 | 5.97 | 19.88 | 10.94 | 2.99 | | |
| 0.135 | 13.74 | 6.08 | 6.54 | 5.47 | 4.95 | 4.63 | 7.25 | 5.95 | 4.01 | 3.73 | | |
| 0.045 | 24.48 | 11.43 | 13.26 | 6.25 | 5.38 | 15.91 | 12.06 | 4.76 | 4.33 | 4.4 | | |
| 0.015 | 4.56 | 7.49 | 6.59 | 9.95 | 5.7 | 6.84 | 4.84 | 4.26 | 5.11 | 5 | | |
| 0.005 | 9.03 | 3.99 | 6.9 | 9.43 | 7.84 | 6.15 | 5.61 | 4.14 | 4.87 | 4.97 | | |
| 0 | 0 | 8.76 | 4.23 | 6.84 | 8.03 | 7.14 | 4.94 | 5.57 | 4.62 | 4.24 | | |

| [DRUG] | | | | | | | | | | | ADDITIVE | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 90.85 | 93.69 | 95.55 | 96.61 | 97.56 | 97.98 | 98.31 | 98.57 | 98.69 | 98.75 | | |
| 0.135 | 76.77 | 83.97 | 88.69 | 91.39 | 93.82 | 94.88 | 95.71 | 96.37 | 96.67 | 96.84 | | |
| 0.045 | 37.72 | 57.03 | 69.69 | 76.92 | 83.42 | 86.28 | 88.49 | 90.26 | 91.08 | 91.52 | | |
| 0.015 | 15.85 | 41.94 | 59.04 | 68.81 | 77.6 | 81.46 | 84.45 | 86.84 | 87.94 | 88.54 | | |
| 0.005 | 6.13 | 35.23 | 54.31 | 65.21 | 75.01 | 79.32 | 82.65 | 85.32 | 86.55 | 87.21 | | |
| 0 | 0 | 31 | 51.33 | 62.94 | 73.38 | 77.97 | 81.52 | 84.36 | 85.67 | 86.38 | | |

| [DRUG] | | | | | | | | | | | SYNERGY | Cmpd |
|---|---|---|---|---|---|---|---|---|---|---|---|---|
| Cmpd 1 | | | | | | | | | | | PLOT (99%) | 2 μM |
| μM | 0 | 0.0001 | 0.0002 | 0.0004 | 0.001 | 0.002 | 0.003 | 0.007 | 0.013 | 0.027 | Bonferroni Adj. | 55% |
| 0.405 | 0 | 0 | 0 | 0 | 0 | 0 | 0 | 0 | 0 | 0 | SYNERGY | 0 |
| 0.135 | 0 | 0 | 0 | 0 | 0 | 0 | 0 | 0 | 0 | 0 | log volume | 0 |
| 0.045 | 0 | 0 | 0 | 0 | 0 | 0 | 0 | 0 | 0 | 0 | | |
| 0.015 | 0 | 0 | 0 | 0 | 0 | 0 | 0 | 0 | 0 | 0 | ANTAGONISM | 0 |
| 0.005 | 0 | 0 | 0 | 0 | 0 | 0 | 0 | 0 | 0 | 0 | log volume | 0 |
| 0 | 0 | 0 | 0 | 0 | 0 | 0 | 0 | 0 | 0 | 0 | | |

TABLE 6C
In vitro Combination of Compound 1 and Compound 2 in presence of
ETV @ 0.001 μM (HepG 2.2.15 cell culture model)

| [DRUG] | | | | | | | | | | | AVERAGE % | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 87.59 | 88.91 | 89.95 | 88.93 | 89.1 | 89.27 | 89.71 | 90.17 | 89.47 | 89.2 | | |
| 0.135 | 75.63 | 79.46 | 81.81 | 84.15 | 84.98 | 88.2 | 87.54 | 88.58 | 88.31 | 88.23 | | |
| 0.045 | 40.32 | 57.17 | 63.29 | 68.58 | 77.62 | 81.5 | 83.2 | 83.34 | 86.01 | 86.75 | | |
| 0.015 | 5.62 | 33.45 | 47.15 | 60.64 | 68.48 | 77.49 | 81.37 | 83.57 | 84.58 | 85.16 | | |
| 0.005 | 2.26 | 26.69 | 41.02 | 56.62 | 69.36 | 75.3 | 78.33 | 82.25 | 84.39 | 84.1 | | |
| 0 | 0 | 32.38 | 47.22 | 59.69 | 69.96 | 75.47 | 80.14 | 82.58 | 83.5 | 85.12 | | |

| [DRUG] | | | | | | | | | | | STANDARD | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | DEVIATION (%) | 2 μM |
| 0.405 | 5.59 | 4.46 | 3.17 | 3.77 | 5.24 | 3.54 | 2.18 | 3.56 | 4.24 | 3.68 | | |
| 0.135 | 10.9 | 8.34 | 8.16 | 7.21 | 6.89 | 4.24 | 3.95 | 4.4 | 3.72 | 4.25 | | |
| 0.045 | 8.5 | 8.97 | 11.66 | 9.67 | 5.51 | 4.74 | 6.46 | 7.85 | 4.89 | 3.72 | | |
| 0.015 | 7.4 | 5.06 | 7.82 | 4.81 | 4.7 | 5.4 | 4.36 | 5.26 | 3.32 | 4.23 | | |
| 0.005 | 12.29 | 9.44 | 6.44 | 6.68 | 2.44 | 2.46 | 5.73 | 5.15 | 4.06 | 4.01 | | |
| 0 | 0 | 5.6 | 1.95 | 3.68 | 5.86 | 6.82 | 5.96 | 6.55 | 5.01 | 3.49 | | |

| [DRUG] | | | | | | | | | | | ADDITIVE | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 87.59 | 91.61 | 93.45 | 95 | 96.27 | 96.96 | 97.54 | 97.84 | 97.95 | 98.15 | | |
| 0.135 | 75.63 | 83.52 | 87.14 | 90.18 | 92.68 | 94.02 | 95.16 | 95.75 | 95.98 | 96.37 | | |
| 0.045 | 40.32 | 59.64 | 68.5 | 75.94 | 82.07 | 85.36 | 88.15 | 89.6 | 90.15 | 91.12 | | |
| 0.015 | 5.62 | 36.18 | 50.19 | 61.96 | 71.65 | 76.85 | 81.26 | 83.56 | 84.43 | 85.96 | | |
| 0.005 | 2.26 | 33.91 | 48.41 | 60.6 | 70.64 | 76.02 | 80.59 | 82.97 | 83.87 | 85.46 | | |
| 0 | 0 | 32.38 | 47.22 | 59.69 | 69.96 | 75.47 | 80.14 | 82.58 | 83.5 | 85.12 | | |

| [DRUG] | | | | | | | | | | | SYNERGY | Cmpd |
|---|---|---|---|---|---|---|---|---|---|---|---|---|
| Cmpd 1 | | | | | | | | | | | PLOT (99%) | 2 μM |
| μM | 0 | 0.0001 | 0.0002 | 0.0004 | 0.001 | 0.002 | 0.003 | 0.007 | 0.013 | 0.027 | Bonferroni Adj. | 55% |
| 0.405 | 0 | 0 | 0 | 0 | 0 | 0 | −2.2274 | 0 | 0 | 0 | SYNERGY | 0 |
| 0.135 | 0 | 0 | 0 | 0 | 0 | 0 | 0 | 0 | 0 | 0 | log volume | 0 |
| 0.045 | 0 | 0 | 0 | 0 | 0 | 0 | 0 | 0 | 0 | 0 | | |
| 0.015 | 0 | 0 | 0 | 0 | 0 | 0 | 0 | 0 | 0 | 0 | ANTAGONISM | −2.23 |
| 0.005 | 0 | 0 | 0 | 0 | 0 | 0 | 0 | 0 | 0 | 0 | log volume | −0.32 |
| 0 | 0 | 0 | 0 | 0 | 0 | 0 | 0 | 0 | 0 | 0 | | |

TABLE 6D
In vitro Combination of Compound 1 and Compound 2 in presence of
ETV @ 0.003 μM (HepG 2.2.15 cell culture model)

| [DRUG] | | | | | | | | | | | AVERAGE % | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 84.43 | 83.2 | 81.71 | 85.17 | 84.72 | 82.28 | 76.36 | 85.46 | 86.46 | 83.26 | | |
| 0.135 | 70.96 | 70.12 | 68.61 | 76.22 | 81.58 | 81.14 | 75.17 | 82.7 | 82.66 | 83.43 | | |
| 0.045 | 38.54 | 49.29 | 53.62 | 65.78 | 71.87 | 74.96 | 78.5 | 83.22 | 81.85 | 81.39 | | |
| 0.015 | 8.24 | 37.1 | 42.96 | 57.79 | 69.37 | 70.1 | 77.01 | 79.87 | 80.03 | 81.84 | | |
| 0.005 | −3.43 | 31 | 44.26 | 57.17 | 64.77 | 76 | 78.55 | 79.45 | 76.09 | 80.69 | | |
| 0 | 0 | 34.1 | 47.55 | 58.27 | 68.96 | 74.34 | 78.72 | 79.98 | 79.68 | 81.99 | | |

| [DRUG] | | | | | | | | | | | STANDARD | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | DEVIATION (%) | 2 μM |
| 0.405 | 5.36 | 7.83 | 10.33 | 5.54 | 6.22 | 7.07 | 21.62 | 5.89 | 4.4 | 7.93 | | |
| 0.135 | 12.42 | 16.73 | 20.97 | 9.03 | 7.41 | 7.14 | 18.22 | 6.9 | 5.65 | 5.66 | | |
| 0.045 | 16.88 | 12.05 | 12.68 | 8.83 | 6.14 | 7.98 | 4.92 | 11.01 | 5.47 | 5.81 | | |
| 0.015 | 15.48 | 3.81 | 2.81 | 7.67 | 4.76 | 4.9 | 7.4 | 6.76 | 4.31 | 5.62 | | |
| 0.005 | 16.99 | 9.94 | 2.95 | 4.25 | 9.34 | 7.49 | 6.06 | 5.59 | 4.84 | 4.81 | | |
| 0 | 0 | 4.46 | 3.52 | 8.62 | 8.84 | 9.12 | 5.59 | 8.07 | 6.57 | 4.98 | | |

| [DRUG] | | | | | | | | | | | ADDITIVE | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 84.43 | 89.74 | 91.83 | 93.5 | 95.17 | 96 | 96.69 | 96.88 | 96.84 | 97.2 | | |
| 0.135 | 70.96 | 80.86 | 84.77 | 87.88 | 90.99 | 92.55 | 93.82 | 94.19 | 94.1 | 94.77 | | |
| 0.045 | 38.54 | 59.5 | 67.76 | 74.35 | 80.92 | 84.23 | 86.92 | 87.7 | 87.51 | 88.93 | | |
| 0.015 | 8.24 | 39.53 | 51.87 | 61.71 | 71.52 | 76.45 | 80.47 | 81.63 | 81.35 | 83.47 | | |
| 0.005 | −3.43 | 31.84 | 45.75 | 56.84 | 67.9 | 73.46 | 77.99 | 79.29 | 78.98 | 81.37 | | |
| 0 | 0 | 34.1 | 47.55 | 58.27 | 68.96 | 74.34 | 78.72 | 79.98 | 79.68 | 81.99 | | |

| [DRUG] | | | | | | | | | | | SYNERGY | Cmpd |
|---|---|---|---|---|---|---|---|---|---|---|---|---|
| Cmpd 1 | | | | | | | | | | | PLOT (99%) | 2 μM |
| μM | 0 | 0.0001 | 0.0002 | 0.0004 | 0.001 | 0.002 | 0.003 | 0.007 | 0.013 | 0.027 | Bonferroni Adj. | 55% |
| 0.405 | 0 | 0 | 0 | 0 | 0 | 0 | 0 | 0 | 0 | 0 | SYNERGY | 0 |
| 0.135 | 0 | 0 | 0 | 0 | 0 | 0 | 0 | 0 | 0 | 0 | log volume | 0 |
| 0.045 | 0 | 0 | 0 | 0 | 0 | 0 | 0 | 0 | 0 | 0 | | |
| 0.015 | 0 | 0 | −1.6883 | 0 | 0 | 0 | 0 | 0 | 0 | 0 | ANTAGONISM | −1.69 |
| 0.005 | 0 | 0 | 0 | 0 | 0 | 0 | 0 | 0 | 0 | 0 | log volume | −0.24 |
| 0 | 0 | 0 | 0 | 0 | 0 | 0 | 0 | 0 | 0 | 0 | | |

TABLE 6E
In vitro Combination of Compound 1 and Compound 2 in presence of
ETV @ 0.009 μM (HepG 2.2.15 cell culture model)

| [DRUG] | | | | | | | | | | | AVERAGE % | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 73.79 | 76.01 | 78.04 | 76.14 | 77.4 | 70.08 | 74.11 | 74.93 | 74.93 | 73.38 | | |
| 0.135 | 62.78 | 68.52 | 69.34 | 71.18 | 72.42 | 73.41 | 73.79 | 75.61 | 76.71 | 76.46 | | |
| 0.045 | 31.13 | 45.74 | 50.45 | 58.24 | 67.07 | 67.43 | 70.92 | 75.16 | 73.6 | 69.7 | | |
| 0.015 | −9.5 | 31.23 | 44.14 | 51.61 | 61.67 | 66.15 | 69.36 | 73.85 | 72.61 | 70.46 | | |
| 0.005 | −0.64 | 25.62 | 36.39 | 52.95 | 55.75 | 64.3 | 67.16 | 71.84 | 73.76 | 73.59 | | |
| 0 | 0 | 22.64 | 37.66 | 52.72 | 59.08 | 66.57 | 69.55 | 71.88 | 71.98 | 72.49 | | |

| [DRUG] | | | | | | | | | | | STANDARD | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | DEVIATION (%) | 2 μM |
| 0.405 | 7.28 | 8.31 | 6.52 | 7.64 | 6.18 | 9.11 | 9.99 | 7.12 | 9.94 | 11.59 | | |
| 0.135 | 9.24 | 10.19 | 9.81 | 9.02 | 8.36 | 9.05 | 10.52 | 6.51 | 6.31 | 5.9 | | |
| 0.045 | 13.96 | 11.37 | 12 | 8.8 | 8.64 | 10.88 | 9.06 | 8.09 | 7.33 | 11.08 | | |
| 0.015 | 15.08 | 7.87 | 10.82 | 10.96 | 9.74 | 8.77 | 9.82 | 7.55 | 9.21 | 10.54 | | |
| 0.005 | 5.97 | 20.51 | 11 | 6.74 | 13.22 | 11.63 | 10.54 | 6.89 | 6.31 | 9.18 | | |
| 0 | 0 | 6.48 | 9.98 | 7.4 | 11.79 | 10.78 | 9.29 | 6.89 | 5.89 | 7.02 | | |

| [DRUG] | | | | | | | | | | | ADDITIVE | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 73.79 | 79.72 | 83.66 | 87.61 | 89.27 | 91.24 | 92.02 | 92.63 | 92.66 | 92.79 | | |
| 0.135 | 62.78 | 71.21 | 76.8 | 82.4 | 84.77 | 87.56 | 88.67 | 89.53 | 89.57 | 89.76 | | |
| 0.045 | 31.13 | 46.72 | 57.07 | 67.44 | 71.82 | 76.98 | 79.03 | 80.63 | 80.7 | 81.05 | | |
| 0.015 | −9.5 | 15.29 | 31.74 | 48.23 | 55.19 | 63.39 | 66.66 | 69.21 | 69.32 | 69.88 | | |
| 0.005 | −0.64 | 22.14 | 37.26 | 52.42 | 58.82 | 66.36 | 69.36 | 71.7 | 71.8 | 72.31 | | |
| 0 | 0 | 22.64 | 37.66 | 52.72 | 59.08 | 66.57 | 69.55 | 71.88 | 71.98 | 72.49 | | |

| [DRUG] | | | | | | | | | | | SYNERGY | Cmpd |
|---|---|---|---|---|---|---|---|---|---|---|---|---|
| Cmpd 1 | | | | | | | | | | | PLOT (99%) | 2 μM |
| μM | 0 | 0.0001 | 0.0002 | 0.0004 | 0.001 | 0.002 | 0.003 | 0.007 | 0.013 | 0.027 | Bonferroni Adj. | 55% |
| 0.405 | 0 | 0 | 0 | 0 | 0 | 0 | 0 | 0 | 0 | 0 | SYNERGY | 0 |
| 0.135 | 0 | 0 | 0 | 0 | 0 | 0 | 0 | 0 | 0 | 0 | log volume | 0 |
| 0.045 | 0 | 0 | 0 | 0 | 0 | 0 | 0 | 0 | 0 | 0 | | |
| 0.015 | 0 | 0 | 0 | 0 | 0 | 0 | 0 | 0 | 0 | 0 | ANTAGONISM | 0 |
| 0.005 | 0 | 0 | 0 | 0 | 0 | 0 | 0 | 0 | 0 | 0 | log volume | 0 |
| 0 | 0 | 0 | 0 | 0 | 0 | 0 | 0 | 0 | 0 | 0 | | |

TABLE 7A
In vitro Combination of Compound 1 and Compound 2 in presence
of TAF @ 0 μM (HepG 2.2.15 cell culture model):

| [DRUG] | | | | | | | | | | | AVERAGE % | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 92.36 | 89.26 | 93.78 | 93.66 | 93.58 | 93.92 | 93.88 | 94.3 | 96.09 | 94.03 | | |
| 0.135 | 79.36 | 84.5 | 85.23 | 88.13 | 87.83 | 90.03 | 91.44 | 92.82 | 92.49 | 92.13 | | |
| 0.045 | 43.93 | 51.64 | 64.87 | 69.37 | 76.15 | 82.29 | 84.93 | 88.08 | 89.97 | 90.42 | | |
| 0.015 | −1.68 | 33.41 | 46.71 | 55.46 | 71.75 | 80.45 | 82.75 | 85.88 | 87.12 | 88.1 | | |
| 0.005 | −3.28 | 28.2 | 33.32 | 53.48 | 68.48 | 77.94 | 80.65 | 85.7 | 86.6 | 88.04 | | |
| 0 | 0 | 31.76 | 43.68 | 58.53 | 71.85 | 78.06 | 83.72 | 85.49 | 89.05 | 87.17 | | |

| [DRUG] | | | | | | | | | | | STANDARD | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | DEVIATION (%) | 2 μM |
| 0.405 | 3.49 | 3.1 | 2.35 | 3.29 | 2.64 | 2.56 | 2.1 | 2.29 | 3.32 | 2.33 | | |
| 0.135 | 13.22 | 9.2 | 9.03 | 6.64 | 7.06 | 5.7 | 4.1 | 3.03 | 3.71 | 3.74 | | |
| 0.045 | 16.68 | 24.69 | 13.85 | 11.2 | 10.8 | 6.25 | 8.15 | 5.68 | 4.92 | 3.72 | | |
| 0.015 | 10.28 | 21.01 | 20.42 | 16.33 | 9.92 | 6.79 | 6.71 | 6.87 | 6.61 | 5.37 | | |
| 0.005 | 30.31 | 15.46 | 25.38 | 21.83 | 12.96 | 9.61 | 8.74 | 6.39 | 4.92 | 4.18 | | |
| 0 | 0 | 17.11 | 17.93 | 17.41 | 10.59 | 9.09 | 7.51 | 6.39 | 3.34 | 5.84 | | |

| [DRUG] | | | | | | | | | | | ADDITIVE | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 92.36 | 94.79 | 95.7 | 96.83 | 97.85 | 98.32 | 98.76 | 98.89 | 99.16 | 99.02 | | |
| 0.135 | 79.36 | 85.92 | 88.38 | 91.44 | 94.19 | 95.47 | 96.64 | 97.01 | 97.74 | 97.35 | | |
| 0.045 | 43.93 | 61.74 | 68.42 | 76.75 | 84.22 | 87.7 | 90.87 | 91.86 | 93.86 | 92.81 | | |
| 0.015 | −1.68 | 30.61 | 42.73 | 57.83 | 71.38 | 77.69 | 83.45 | 85.25 | 88.87 | 86.95 | | |
| 0.005 | −3.28 | 29.52 | 41.83 | 57.17 | 70.93 | 77.34 | 83.19 | 85.01 | 88.69 | 86.75 | | |
| 0 | 0 | 31.76 | 43.68 | 58.53 | 71.85 | 78.06 | 83.72 | 85.49 | 89.05 | 87.17 | | |

| [DRUG] | | | | | | | | | | | SYNERGY | Cmpd |
|---|---|---|---|---|---|---|---|---|---|---|---|---|
| Cmpd 1 | | | | | | | | | | | PLOT (99%) | 2 μM |
| μM | 0 | 0.0001 | 0.0002 | 0.0004 | 0.001 | 0.002 | 0.003 | 0.007 | 0.013 | 0.027 | Bonferroni Adj. | 55% |
| 0.405 | 0 | 0 | 0 | 0 | 0 | 0 | 0 | 0 | 0 | 0 | SYNERGY | 0 |
| 0.135 | 0 | 0 | 0 | 0 | 0 | 0 | 0 | 0 | 0 | 0 | log volume | 0 |
| 0.045 | 0 | 0 | 0 | 0 | 0 | 0 | 0 | 0 | 0 | 0 | | |
| 0.015 | 0 | 0 | 0 | 0 | 0 | 0 | 0 | 0 | 0 | 0 | ANTAGONISM | 0 |
| 0.005 | 0 | 0 | 0 | 0 | 0 | 0 | 0 | 0 | 0 | 0 | log volume | 0 |
| 0 | 0 | 0 | 0 | 0 | 0 | 0 | 0 | 0 | 0 | 0 | | |

TABLE 7B
In vitro Combination of Compound 1 and Compound 2 in presence of
TAF @ 0.003 μM (HepG 2.2.15 cell culture model)

| [DRUG] | | | | | | | | | | | AVERAGE % | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 92.54 | 92.23 | 94.01 | 94.05 | 94.23 | 94.07 | 94.57 | 94.4 | 94.05 | 94.22 | | |
| 0.135 | 84.29 | 84.64 | 87.29 | 89.4 | 90.58 | 91.7 | 91.94 | 92.82 | 93.18 | 92.36 | | |
| 0.045 | 47.14 | 58.14 | 65.31 | 70.8 | 79.1 | 82.87 | 85.64 | 88.11 | 90.65 | 90.42 | | |
| 0.015 | 21.04 | 41.43 | 53.21 | 58.57 | 70.89 | 77.84 | 83.3 | 86.07 | 87.76 | 88.7 | | |
| 0.005 | 10.8 | 35.94 | 44.43 | 55.11 | 69.57 | 77.35 | 81.66 | 84.7 | 86.63 | 88.06 | | |
| 0 | 0 | 34.75 | 46.19 | 61.14 | 71 | 78.89 | 82.49 | 86.4 | 88.62 | 87.68 | | |

| [DRUG] | | | | | | | | | | | STANDARD | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | DEVIATION (%) | 2 μM |
| 0.405 | 2.81 | 2.07 | 2.07 | 1.9 | 2.14 | 1.81 | 1.33 | 1.67 | 2.25 | 1.4 | | |
| 0.135 | 6.36 | 7.74 | 4.88 | 3.77 | 3.97 | 2.23 | 4.19 | 2.21 | 2.02 | 2.71 | | |
| 0.045 | 14.03 | 12.37 | 9 | 7.74 | 5.24 | 4.81 | 3.36 | 3.32 | 2.3 | 3.09 | | |
| 0.015 | 6.56 | 14.7 | 4.74 | 8.68 | 6.76 | 6.91 | 4.25 | 2.18 | 3.48 | 3.03 | | |
| 0.005 | 9.78 | 8.17 | 12.69 | 10.75 | 7.95 | 5.49 | 5.87 | 4.53 | 3.93 | 4.21 | | |
| 0 | 0 | 10.05 | 7.84 | 7.37 | 6.51 | 7.31 | 4.58 | 4.22 | 3.3 | 3.66 | | |

| [DRUG] | | | | | | | | | | | ADDITIVE | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 92.54 | 95.13 | 95.99 | 97.1 | 97.84 | 98.43 | 98.69 | 98.99 | 99.15 | 99.08 | | |
| 0.135 | 84.29 | 89.75 | 91.55 | 93.9 | 95.44 | 96.68 | 97.25 | 97.86 | 98.21 | 98.06 | | |
| 0.045 | 47.14 | 65.51 | 71.56 | 79.46 | 84.67 | 88.84 | 90.74 | 92.81 | 93.98 | 93.49 | | |
| 0.015 | 21.04 | 48.48 | 57.51 | 69.32 | 77.1 | 83.33 | 86.17 | 89.26 | 91.01 | 90.27 | | |
| 0.005 | 10.8 | 41.8 | 52 | 65.34 | 74.13 | 81.17 | 84.38 | 87.87 | 89.85 | 89.01 | | |
| 0 | 0 | 34.75 | 46.19 | 61.14 | 71 | 78.89 | 82.49 | 86.4 | 88.62 | 87.68 | | |

| [DRUG] | | | | | | | | | | | SYNERGY | Cmpd |
|---|---|---|---|---|---|---|---|---|---|---|---|---|
| Cmpd 1 | | | | | | | | | | | PLOT (99%) | 2 μM |
| μM | 0 | 0.0001 | 0.0002 | 0.0004 | 0.001 | 0.002 | 0.003 | 0.007 | 0.013 | 0.027 | Bonferroni Adj. | 55% |
| 0.405 | 0 | 0 | 0 | 0 | 0 | 0 | −0.7019 | −0.2981 | 0 | −1.262 | SYNERGY | 0 |
| 0.135 | 0 | 0 | 0 | 0 | 0 | 0 | 0 | 0 | 0 | 0 | log volume | 0 |
| 0.045 | 0 | 0 | 0 | 0 | 0 | 0 | 0 | 0 | 0 | 0 | | |
| 0.015 | 0 | 0 | 0 | 0 | 0 | 0 | 0 | 0 | 0 | 0 | ANTAGONISM | −2.26 |
| 0.005 | 0 | 0 | 0 | 0 | 0 | 0 | 0 | 0 | 0 | 0 | log volume | −0.32 |
| 0 | 0 | 0 | 0 | 0 | 0 | 0 | 0 | 0 | 0 | 0 | | |

TABLE 7C
In vitro Combination of Compound 1 and Compound 2 in presence of TAF @ 0.010 μM (HepG 2.2.15 cell culture model)

| [DRUG] | | | | | | | | | | | AVERAGE % | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 92.12 | 91.11 | 91.83 | 92.25 | 92.28 | 91.96 | 92.49 | 92.2 | 92.74 | 92.13 | | |
| 0.135 | 78.24 | 82.86 | 84.26 | 86.49 | 88.58 | 88.35 | 90.42 | 90.91 | 90.3 | 90.34 | | |
| 0.045 | 39.8 | 53.36 | 71.18 | 69.51 | 74.41 | 81.16 | 85.07 | 89.71 | 88.25 | 87.32 | | |
| 0.015 | 10.5 | 36.62 | 39.32 | 59.98 | 62.3 | 73.9 | 79.91 | 83.78 | 86.87 | 87.23 | | |
| 0.005 | −4.68 | 32.77 | 31.9 | 55.51 | 64.57 | 76.13 | 80.17 | 82.49 | 85.7 | 86.7 | | |
| 0 | 0 | 26.66 | 44.2 | 55.09 | 68.37 | 73.41 | 79.06 | 82.74 | 85.57 | 85.51 | | |

| [DRUG] | | | | | | | | | | | STANDARD | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | DEVIATION (%) | 2 μM |
| 0.405 | 5.51 | 2.29 | 1.89 | 1.9 | 1.95 | 1.36 | 2.02 | 2.21 | 2.4 | 2.18 | | |
| 0.135 | 5.05 | 6.58 | 4.45 | 4.06 | 3.18 | 3.96 | 2.33 | 2.17 | 3.25 | 3.58 | | |
| 0.045 | 2.83 | 11.8 | 24.53 | 6.78 | 7.98 | 5.88 | 3.83 | 8.31 | 3.26 | 3.83 | | |
| 0.015 | 11.9 | 6.19 | 17.75 | 6.71 | 10.6 | 5.48 | 5.1 | 4.79 | 3.43 | 3.95 | | |
| 0.005 | 11.86 | 14 | 12.49 | 9.44 | 9.12 | 5.94 | 6.34 | 5.4 | 3.77 | 3.16 | | |
| 0 | 0 | 9.5 | 6.2 | 12.4 | 7.8 | 7.37 | 7.48 | 4.87 | 4.36 | 5.3 | | |

| [DRUG] | | | | | | | | | | | ADDITIVE | Cmpd |
|---|---|---|---|---|---|---|---|---|---|---|---|---|
| Cmpnd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 92.12 | 94.22 | 95.6 | 96.46 | 97.51 | 97.9 | 98.35 | 98.64 | 98.86 | 98.86 | | |
| 0.135 | 78.24 | 84.04 | 87.86 | 90.23 | 93.12 | 94.21 | 95.44 | 96.24 | 96.86 | 96.85 | | |
| 0.045 | 39.8 | 55.85 | 66.41 | 72.96 | 80.96 | 83.99 | 87.39 | 89.61 | 91.31 | 91.28 | | |
| 0.015 | 10.5 | 34.36 | 50.06 | 59.81 | 71.69 | 76.2 | 81.26 | 84.55 | 87.09 | 87.03 | | |
| 0.005 | −4.68 | 23.23 | 41.59 | 52.99 | 66.89 | 72.17 | 78.08 | 81.93 | 84.89 | 84.83 | | |
| 0 | 0 | 26.66 | 44.2 | 55.09 | 68.37 | 73.41 | 79.06 | 82.74 | 85.57 | 85.51 | | |

| [DRUG] | | | | | | | | | | | SYNERGY PLOT (99%) | Cmpd 2 μM |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | Bonferroni Adj. | 55% |
| 0.405 | 0 | 0 | 0 | 0 | −0.2185 | −2.4448 | −0.6686 | 0.7603 | 0 | −1.1274 | SYNERGY | 0 |
| 0.135 | 0 | 0 | 0 | 0 | 0 | 0 | 0 | 0 | 0 | 0 | log volume | 0 |
| 0.045 | 0 | 0 | 0 | 0 | 0 | 0 | 0 | 0 | 0 | 0 | ANTAGONISM | −5.22 |
| 0.015 | 0 | 0 | 0 | 0 | 0 | 0 | 0 | 0 | 0 | 0 | log volume | −0.75 |
| 0.005 | 0 | 0 | 0 | 0 | 0 | 0 | 0 | 0 | 0 | 0 | | |
| 0 | 0 | 0 | 0 | 0 | 0 | 0 | 0 | 0 | 0 | 0 | | |

TABLE 7D
In vitro Combination of Compound 1 and Compound 2 in presence of TAF @ 0.030 μM (HepG 2.2.15 cell culture model)

| [DRUG] | | | | | | | | | | | AVERAGE % | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 86.65 | 89.62 | 90.18 | 90.55 | 90.7 | 90.08 | 91.19 | 90.37 | 82.41 | 88.03 | | |
| 0.135 | 77.01 | 75.06 | 83.03 | 84.22 | 85.21 | 87.11 | 89.72 | 89.2 | 88.94 | 89.49 | | |
| 0.045 | 34.06 | 52.43 | 62.95 | 67.02 | 74.91 | 78.01 | 82.86 | 85.99 | 87.24 | 86.33 | | |
| 0.015 | 0.74 | 33.94 | 46.16 | 54.5 | 75.11 | 75.73 | 80.45 | 83.14 | 84 | 84.87 | | |
| 0.005 | −3.39 | 23.58 | 35.13 | 49.76 | 62.67 | 70.39 | 75.48 | 84.11 | 83.32 | 83.23 | | |
| 0 | 0 | 20.58 | 47.62 | 48.28 | 66.74 | 72.05 | 78.34 | 83.13 | 82.78 | 78.48 | | |

| [DRUG] | | | | | | | | | | | STANDARD | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | DEVIATION (%) | 2 μM |
| 0.405 | 7.03 | 3.75 | 2.81 | 2.38 | 2 | 2.89 | 2.69 | 1.86 | 17.4 | 3.38 | | |
| 0.135 | 9.87 | 16.05 | 5.46 | 5.34 | 6.11 | 3.43 | 6.78 | 3.04 | 2.3 | 2.16 | | |
| 0.045 | 25.01 | 15.63 | 12.22 | 13.96 | 6.07 | 8.05 | 5.81 | 4.58 | 3.44 | 3.99 | | |
| 0.015 | 19.62 | 15.22 | 9.88 | 18.17 | 17.62 | 8.25 | 5.55 | 5.25 | 5.08 | 5.95 | | |
| 0.005 | 10.99 | 17.07 | 16.83 | 17.77 | 14.16 | 12.4 | 7.48 | 11.89 | 5.72 | 5.57 | | |
| 0 | 0 | 9.97 | 37.84 | 15.9 | 9.23 | 10.97 | 7.67 | 3.76 | 3.94 | 10.66 | | |

| [DRUG] | | | | | | | | | | | ADDITIVE | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 86.65 | 89.4 | 93.01 | 93.1 | 95.56 | 96.27 | 97.11 | 97.75 | 97.7 | 97.13 | | |
| 0.135 | 77.01 | 81.74 | 87.96 | 88.11 | 92.35 | 93.57 | 95.02 | 96.12 | 96.04 | 95.05 | | |
| 0.045 | 34.06 | 47.63 | 65.46 | 65.9 | 78.07 | 81.57 | 85.72 | 88.88 | 88.65 | 85.81 | | |
| 0.015 | 0.74 | 21.17 | 48.01 | 48.66 | 66.99 | 72.26 | 78.5 | 83.25 | 82.91 | 78.64 | | |
| 0.005 | −3.39 | 17.89 | 45.84 | 46.53 | 65.61 | 71.1 | 77.61 | 82.56 | 82.2 | 77.75 | | |
| 0 | 0 | 20.58 | 47.62 | 48.28 | 66.74 | 72.05 | 78.34 | 83.13 | 82.78 | 78.48 | | |

| [DRUG] | | | | | | | | | | | SYNERGY PLOT (99%) | Cmpd 2 μM |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | Bonferroni Adj. | 55% |
| 0.405 | 0 | 0 | 0 | 0 | 0 | 0 | 0 | −2.5998 | 0 | −0.4134 | SYNERGY | 0 |
| 0.135 | 0 | 0 | 0 | 0 | 0 | 0 | 0 | 0 | −1.189 | −0.0088 | log volume | 0 |
| 0.045 | 0 | 0 | 0 | 0 | 0 | 0 | 0 | 0 | 0 | 0 | ANTAGONISM | −4.21 |
| 0.015 | 0 | 0 | 0 | 0 | 0 | 0 | 0 | 0 | 0 | 0 | log volume | −0.6 |
| 0.005 | 0 | 0 | 0 | 0 | 0 | 0 | 0 | 0 | 0 | 0 | | |
| 0 | 0 | 0 | 0 | 0 | 0 | 0 | 0 | 0 | 0 | 0 | | |

TABLE 7E
In vitro Combination of Compound 1 and Compound 2 in presence of TAF @ 0.100 μM (HepG 2.2.15 cell culture model)

| [DRUG] | | | | | | | | | | | AVERAGE % | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 70.42 | 80.33 | 80.28 | 80.41 | 81.23 | 79.12 | 80.07 | 78.91 | 78.47 | 82.74 | | |
| 0.135 | 68.3 | 70.51 | 74.44 | 77.56 | 77.63 | 79.67 | 78.53 | 78.22 | 76.75 | 80.87 | | |
| 0.045 | 34.09 | 52.11 | 58.08 | 66.49 | 71.94 | 72.69 | 74.25 | 74.74 | 80.81 | 73.07 | | |
| 0.015 | 10.14 | 36.43 | 47.88 | 57.43 | 65.54 | 72 | 73.99 | 77.79 | 81.05 | 74.33 | | |
| 0.005 | 4.8 | 34.37 | 40.08 | 54.47 | 60.5 | 64.5 | 68.69 | 73.19 | 78.92 | 71.8 | | |
| 0 | 0 | 29.51 | 40.93 | 48.41 | 60.51 | 74.39 | 67.85 | 71.06 | 77.24 | 78.1 | | |

| [DRUG] | | | | | | | | | | | STANDARD | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | DEVIATION (%) | 2 μM |
| 0.405 | 17.64 | 4.48 | 3.25 | 6.14 | 4.1 | 3.86 | 5.2 | 4.72 | 4.88 | 5.85 | | |
| 0.135 | 9.29 | 7.24 | 6.66 | 7.13 | 4.06 | 5.61 | 6.6 | 5.34 | 4.66 | 6.15 | | |
| 0.045 | 8.47 | 9.27 | 8.55 | 6.02 | 7.9 | 7.5 | 7.49 | 6.62 | 7.15 | 18.68 | | |
| 0.015 | 5.8 | 6.47 | 4.73 | 8.89 | 10.05 | 8.19 | 7.05 | 12.67 | 7.36 | 4.52 | | |
| 0.005 | 8.89 | 5.57 | 18.08 | 8.67 | 10.82 | 9.21 | 6.58 | 5.98 | 8.19 | 8.5 | | |
| 0 | 0 | 16.91 | 9.78 | 6.85 | 7.29 | 13.1 | 7.78 | 7.94 | 9.92 | 9.79 | | |

| [DRUG] | | | | | | | | | | | ADDITIVE | Cmpd |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | INHIBITION | 2 μM |
| 0.405 | 70.42 | 79.15 | 82.53 | 84.74 | 88.32 | 92.42 | 90.49 | 91.44 | 93.27 | 93.52 | | |
| 0.135 | 68.3 | 77.65 | 81.27 | 83.65 | 87.48 | 91.88 | 89.81 | 90.83 | 92.79 | 93.06 | | |
| 0.045 | 34.09 | 53.54 | 61.07 | 66 | 73.97 | 83.12 | 78.81 | 80.93 | 85 | 85.57 | | |
| 0.015 | 10.14 | 36.66 | 46.92 | 53.64 | 64.51 | 76.99 | 71.11 | 73.99 | 79.55 | 80.32 | | |
| 0.005 | 4.8 | 32.89 | 43.77 | 50.89 | 62.41 | 75.62 | 69.39 | 72.45 | 78.33 | 79.15 | | |
| 0 | 0 | 29.51 | 40.93 | 48.41 | 60.51 | 74.39 | 67.85 | 71.06 | 77.24 | 78.1 | | |

| [DRUG] | | | | | | | | | | | SYNERGY PLOT (99%) | Cmpd 2 μM |
|---|---|---|---|---|---|---|---|---|---|---|---|---|
| Cmpd 1 μM | 0 | 0.0001 | 0.0002 | 0.0004 | 0.001 | 0.002 | 0.003 | 0.007 | 0.013 | 0.027 | Bonferroni Adj. | 55% |
| 0.405 | 0 | 0 | 0 | 0 | 0 | −3.3798 | 0 | −0.3996 | −2.2584 | 0 | SYNERGY | 0 |
| 0.135 | 0 | 0 | 0 | 0 | 0 | 0 | 0 | 0 | −4.0638 | 0 | log volume | 0 |
| 0.045 | 0 | 0 | 0 | 0 | 0 | 0 | 0 | 0 | 0 | 0 | ANTAGONISM | −10.1 |
| 0.015 | 0 | 0 | 0 | 0 | 0 | 0 | 0 | 0 | 0 | 0 | log volume | −1.45 |
| 0.005 | 0 | 0 | 0 | 0 | 0 | 0 | 0 | 0 | 0 | 0 | | |
| 0 | 0 | 0 | 0 | 0 | 0 | 0 | 0 | 0 | 0 | 0 | | |

TABLE 8A
In vitro Combination of Compound 1 and Compound 2 in presence of TDF @ 0 μM (HepG 2.2.15 cell culture model):

| [DRUG] | | | | | | | | | | | AVERAGE % | Cmpd |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | INHIBITION | 2 μM |
| 0.405 | 75.79 | 81.8 | 80.58 | 80.73 | 80.98 | 80.24 | 85.63 | 82.6 | 75.36 | 84.9 | | |
| 0.135 | 63.83 | 65.89 | 68.04 | 66 | 74.84 | 73.78 | 79.45 | 79.15 | 80.6 | 81.37 | | |
| 0.045 | 24.86 | 31.17 | 31.89 | 42.58 | 52.25 | 57.58 | 66.85 | 73.79 | 77.95 | 78.13 | | |
| 0.015 | −3.72 | 1.48 | 15 | 19.1 | 33.75 | 50.92 | 66.2 | 68.23 | 76.65 | 77.21 | | |
| 0.005 | −24 | −0.48 | 10.19 | 18.77 | 31.84 | 45.76 | 58.43 | 68.28 | 72.54 | 77.09 | | |
| 0 | 0 | 7.15 | 9.56 | 23.65 | 36.25 | 40.9 | 64.52 | 69.76 | 74.45 | 77.46 | | |

| [DRUG] | | | | | | | | | | | STANDARD | Cmpd |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | DEVIATION (%) | 2 μM |
| 0.405 | 15.03 | 6.43 | 3.9 | 5.21 | 8.58 | 11.45 | 2.75 | 6.92 | 18.71 | 3.34 | | |
| 0.135 | 24.72 | 21 | 16.13 | 20.63 | 10.99 | 16.29 | 9.41 | 9.2 | 7.49 | 8.21 | | |
| 0.045 | 23.14 | 12.18 | 13.32 | 16.16 | 11.81 | 7.75 | 13.62 | 8.4 | 7.42 | 8.12 | | |
| 0.015 | 10.85 | 15.78 | 5.77 | 10.95 | 8.38 | 10.79 | 9.36 | 8.73 | 7.9 | 6.11 | | |
| 0.005 | 39.54 | 20.8 | 9.42 | 9.9 | 15.53 | 9.38 | 8.81 | 8.77 | 12.05 | 8.79 | | |
| 0 | 0 | 8.29 | 14.51 | 13.03 | 10.76 | 12.96 | 7.99 | 8.19 | 7.69 | 7.81 | | |

| [DRUG] | | | | | | | | | | | ADDITIVE | Cmpd |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | INHIBITION | 2 μM |
| 0.405 | 75.79 | 77.52 | 78.1 | 81.52 | 84.57 | 85.69 | 91.41 | 92.68 | 93.81 | 94.54 | | |
| 0.135 | 63.83 | 66.42 | 67.29 | 72.38 | 76.94 | 78.62 | 87.17 | 89.06 | 90.76 | 91.85 | | |
| 0.045 | 24.86 | 30.23 | 32.04 | 42.63 | 52.1 | 55.59 | 73.34 | 77.28 | 80.8 | 83.06 | | |
| 0.015 | −3.72 | 3.7 | 6.2 | 20.81 | 33.88 | 38.7 | 63.2 | 68.64 | 73.5 | 76.62 | | |
| 0.005 | −24 | −15.13 | −12.15 | 5.33 | 20.95 | 26.72 | 56 | 62.5 | 68.32 | 72.05 | | |
| 0 | 0 | 7.15 | 9.56 | 23.65 | 36.25 | 40.9 | 64.52 | 69.76 | 74.45 | 77.46 | | |

| [DRUG] | | | | | | | | | | | SYNERGY PLOT (99%) | Cmpd 2 μM |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | Bonferroni Adj. | 55% |
| 0.405 | 0 | 0 | 0 | 0 | 0 | 0 | 0 | 0 | 0 | −1.0562 | SYNERGY | 0 |
| 0.135 | 0 | 0 | 0 | 0 | 0 | 0 | 0 | 0 | 0 | 0 | log volume | 0 |
| 0.045 | 0 | 0 | 0 | 0 | 0 | 0 | 0 | 0 | 0 | 0 | ANTAGONISM | −1.06 |
| 0.015 | 0 | 0 | 0 | 0 | 0 | 0 | 0 | 0 | 0 | 0 | log volume | −0.15 |
| 0.005 | 0 | 0 | 0 | 0 | 0 | 0 | 0 | 0 | 0 | 0 | | |
| 0 | 0 | 0 | 0 | 0 | 0 | 0 | 0 | 0 | 0 | 0 | | |

TABLE 8B
In vitro Combination of Compound 1 and Compound 2 in presence of TDF @ 0.010 μM (HepG 2.2.15 cell culture model)

| [DRUG] | | | | | | | | | | | AVERAGE % | Cmpd |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | INHIBITION | 2 μM |
| 0.405 | 85.01 | 84.1 | 84.29 | 86.25 | 85.87 | 86.1 | 82.18 | 85.32 | 86.65 | 86.1 | | |
| 0.135 | 70.97 | 76.26 | 73.27 | 69.17 | 74.52 | 65.29 | 80.64 | 76.87 | 82.98 | 84.01 | | |
| 0.045 | 26.17 | 40.22 | 46.49 | 53.32 | 56.33 | 65.28 | 75.08 | 76.03 | 81.29 | 82.36 | | |
| 0.015 | 7.04 | 19.65 | 25.23 | 26.14 | 46.22 | 55.19 | 63.73 | 75.48 | 76.39 | 73.19 | | |
| 0.005 | 4.45 | 20.74 | 24.34 | 33.46 | 38.76 | 58.8 | 65.04 | 60.61 | 78.81 | 81.36 | | |
| 0 | 0 | 11.04 | 22.15 | 33.78 | 40.6 | 52.11 | 67.85 | 72.62 | 78.26 | 78.12 | | |

| [DRUG] | | | | | | | | | | | STANDARD | Cmpd |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | DEVIATION (%) | 2 μM |
| 0.405 | 3.04 | 3.57 | 4.01 | 2.53 | 3.1 | 2.11 | 7.26 | 2.65 | 2.73 | 2.6 | | |
| 0.135 | 10.03 | 6.79 | 7.96 | 10.38 | 8.08 | 20.21 | 4.18 | 12.33 | 3.36 | 4.95 | | |
| 0.045 | 6.75 | 7.38 | 17.27 | 12.34 | 6.78 | 8.19 | 7.16 | 4.85 | 4.14 | 3.42 | | |
| 0.015 | 11.01 | 10.82 | 11.79 | 10.34 | 7.76 | 9.2 | 12.21 | 5.85 | 6.29 | 10.38 | | |
| 0.005 | 9.94 | 8.14 | 13.12 | 13.06 | 12.53 | 5.43 | 6.72 | 17.48 | 4.87 | 4.16 | | |
| 0 | 0 | 20.41 | 17.63 | 16.5 | 11.86 | 4.72 | 7.13 | 4.53 | 4.91 | 7.34 | | |

| [DRUG] | | | | | | | | | | | ADDITIVE | Cmpd |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | INHIBITION | 2 μM |
| 0.405 | 85.01 | 86.66 | 88.33 | 90.07 | 91.1 | 92.82 | 95.18 | 95.9 | 96.74 | 96.72 | | |
| 0.135 | 70.97 | 74.17 | 77.4 | 80.78 | 82.76 | 86.1 | 90.67 | 92.05 | 93.69 | 93.65 | | |
| 0.045 | 26.17 | 34.32 | 42.52 | 51.11 | 56.14 | 64.64 | 76.26 | 79.79 | 83.95 | 83.85 | | |
| 0.015 | 7.04 | 17.3 | 27.63 | 38.44 | 44.78 | 55.48 | 70.11 | 74.55 | 79.79 | 79.66 | | |
| 0.005 | 4.45 | 15 | 25.61 | 36.73 | 43.24 | 54.24 | 69.28 | 73.84 | 79.23 | 79.09 | | |
| 0 | 0 | 11.04 | 22.15 | 33.78 | 40.6 | 52.11 | 67.85 | 72.62 | 78.26 | 78.12 | | |

| [DRUG] | | | | | | | | | | | SYNERGY PLOT (99%) | Cmpd 2 μM |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | Bonferroni Adj. | 55% |
| 0.405 | 0 | 0 | 0 | 0 | 0 | −1.2973 | 0 | −3.7695 | −3.0739 | −3.938 | SYNERGY | 0 |
| 0.135 | 0 | 0 | 0 | 0 | 0 | 0 | 0 | 0 | −2.0748 | 0 | log volume | 0 |
| 0.045 | 0 | 0 | 0 | 0 | 0 | 0 | 0 | 0 | 0 | 0 | ANTAGONISM | −14.15 |
| 0.015 | 0 | 0 | 0 | 0 | 0 | 0 | 0 | 0 | 0 | 0 | log volume | −2.03 |
| 0.005 | 0 | 0 | 0 | 0 | 0 | 0 | 0 | 0 | 0 | 0 | | |
| 0 | 0 | 0 | 0 | 0 | 0 | 0 | 0 | 0 | 0 | 0 | | |

TABLE 8C
In vitro Combination of Compound 1 and Compound 2 in presence of TDF @ 0.030 μM (HepG 2.2.15 cell culture model)

| [DRUG] | | | | | | | | | | | AVERAGE % | Cmpd |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | INHIBITION | 2 μM |
| 0.405 | 78.32 | 82.31 | 79.2 | 81.28 | 83.35 | 82.8 | 81.44 | 81.94 | 83.55 | 80.19 | | |
| 0.135 | 69.08 | 72.44 | 71.87 | 72.52 | 76.71 | 78.83 | 79.82 | 82.24 | 76.92 | 78.95 | | |
| 0.045 | 38.33 | 37.71 | 43.05 | 44.71 | 57.57 | 32.94 | 63.79 | 77.51 | 79.84 | 80.24 | | |
| 0.015 | 14.61 | 23 | 23.63 | 27.73 | 42.82 | 32.46 | 63.25 | 69.48 | 62.3 | 62.73 | | |
| 0.005 | 20.54 | 16.78 | 16.82 | 30.05 | 41.76 | 49.49 | 69.83 | 73.7 | 75.95 | 62.9 | | |
| 0 | 0 | 17.6 | 19.62 | 29.05 | 47.01 | 56.34 | 64.2 | 68.67 | 76.38 | 74.97 | | |

| [DRUG] | | | | | | | | | | | STANDARD | Cmpd |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | DEVIATION (%) | 2 μM |
| 0.405 | 5.39 | 1.35 | 5.59 | 3.27 | 1.45 | 2.84 | 2.29 | 3.94 | 2.52 | 5.13 | | |
| 0.135 | 3.69 | 3.05 | 3.16 | 2.81 | 4.04 | 4.77 | 3.53 | 2.52 | 12.41 | 5.35 | | |
| 0.045 | 4.21 | 7.59 | 2.65 | 21.15 | 3.93 | 40.9 | 16.11 | 3.04 | 4.29 | 2.24 | | |
| 0.015 | 16.21 | 5.28 | 21.35 | 8.74 | 7.14 | 19.67 | 11.78 | 3.48 | 27.81 | 32.58 | | |
| 0.005 | 5.83 | 9.35 | 10.87 | 6.19 | 5.18 | 7.57 | 3.84 | 1.69 | 3.71 | 32.33 | | |
| 0 | 0 | 15.92 | 7.91 | 6.77 | 3.66 | 7.5 | 2.78 | 3.36 | 3.45 | 4.27 | | |

| [DRUG] | | | | | | | | | | | ADDITIVE | Cmpd |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | INHIBITION | 2 μM |
| 0.405 | 78.32 | 82.14 | 82.57 | 84.62 | 88.51 | 90.53 | 92.24 | 93.21 | 94.88 | 94.57 | | |
| 0.135 | 69.08 | 74.52 | 75.15 | 78.06 | 83.62 | 86.5 | 88.93 | 90.31 | 92.7 | 92.26 | | |
| 0.045 | 38.33 | 49.18 | 50.43 | 56.25 | 67.32 | 73.07 | 77.92 | 80.68 | 85.43 | 84.56 | | |
| 0.015 | 14.61 | 29.64 | 31.36 | 39.42 | 54.75 | 62.72 | 69.43 | 73.25 | 79.83 | 78.63 | | |
| 0.005 | 20.54 | 34.52 | 36.13 | 43.62 | 57.89 | 65.31 | 71.55 | 75.11 | 81.23 | 80.11 | | |
| 0 | 0 | 17.6 | 19.62 | 29.05 | 47.01 | 56.34 | 64.2 | 68.67 | 76.38 | 74.97 | | |

| [DRUG] | | | | | | | | | | | SYNERGY PLOT (99%) | Cmpd 2 μM |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | Bonferroni Adj. | 55% |
| 0.405 | 0 | 0 | 0 | 0 | −1.4335 | −0.4312 | −4.9147 | −1.1442 | −4.8536 | −1.1959 | SYNERGY | 0 |
| 0.135 | 0 | 0 | 0 | 0 | 0 | 0 | −0.0379 | −1.5936 | 0 | 0 | log volume | 0 |
| 0.045 | 0 | 0 | −0.5695 | 0 | 0 | 0 | 0 | 0 | 0 | 0 | ANTAGONISM | −18.99 |
| 0.015 | 0 | 0 | 0 | 0 | 0 | 0 | 0 | 0 | 0 | 0 | log volume | −2.73 |
| 0.005 | 0 | 0 | 0 | 0 | −2.8174 | 0 | 0 | 0 | 0 | 0 | | |
| 0 | 0 | 0 | 0 | 0 | 0 | 0 | 0 | 0 | 0 | 0 | | |

TABLE 8D
In vitro Combination of Compound 1 and Compound 2 in presence of TDF @ 0.100 μM (HepG 2.2.15 cell culture model)

| [DRUG] | | | | | | | | | | | AVERAGE % | Cmpd |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | INHIBITION | 2 μM |
| 0.405 | 74.42 | 74.71 | 75.63 | 78.69 | 78.07 | 66.67 | 78.02 | 79 | 79.02 | 75.07 | | |
| 0.135 | 64.15 | 70.9 | 72.16 | 70.99 | 72.27 | 71.73 | 78.19 | 74.57 | 75.03 | 64.52 | | |
| 0.045 | 35.43 | 44.16 | 43.96 | 50.84 | 51.51 | 60.32 | 69.78 | 72.62 | 63.97 | 74.38 | | |
| 0.015 | 27.03 | 30.67 | 36.2 | 33.26 | 52.01 | 54.62 | 67.22 | 68.5 | 73.73 | 77.33 | | |
| 0.005 | 9.81 | 24.6 | 31.6 | 6.5 | 42.13 | 52.22 | 62.97 | 66.1 | 68.88 | 69.79 | | |
| 0 | 0 | 16.22 | 26.58 | 28.6 | 24.38 | 47.23 | 41.8 | 62.15 | 70.15 | 68.94 | | |

| [DRUG] | | | | | | | | | | | STANDARD | Cmpd |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | DEVIATION (%) | 2 μM |
| 0.405 | 13.6 | 12.72 | 13.92 | 9.47 | 11.36 | 18.54 | 9.1 | 7.05 | 9.74 | 10.25 | | |
| 0.135 | 18.23 | 12.64 | 9.44 | 11.46 | 13.5 | 13.6 | 8.46 | 10 | 9.96 | 14.15 | | |
| 0.045 | 18.13 | 13.91 | 12.52 | 12.15 | 23.85 | 15.91 | 11.4 | 8.59 | 20.27 | 7.1 | | |
| 0.015 | 13.33 | 15.38 | 14.79 | 17.37 | 11.58 | 13.68 | 11.16 | 10.4 | 8.38 | 4.46 | | |
| 0.005 | 18.65 | 14.89 | 12.25 | 67.15 | 13.83 | 10.43 | 13.85 | 15.78 | 10.53 | 16.59 | | |
| 0 | 0 | 8.57 | 12.94 | 18.96 | 42.87 | 14.69 | 36.25 | 20.99 | 13.64 | 14.5 | | |

| [DRUG] | | | | | | | | | | | ADDITIVE | Cmpd |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | INHIBITION | 2 μM |
| 0.405 | 74.42 | 78.57 | 81.22 | 81.74 | 80.66 | 86.5 | 85.11 | 90.32 | 92.36 | 92.05 | | |
| 0.135 | 64.15 | 69.96 | 73.68 | 74.4 | 72.89 | 81.08 | 79.14 | 86.43 | 89.3 | 88.86 | | |
| 0.045 | 35.43 | 45.9 | 52.59 | 53.9 | 51.17 | 65.93 | 62.42 | 75.56 | 80.73 | 79.94 | | |
| 0.015 | 27.03 | 38.87 | 46.43 | 47.9 | 44.82 | 61.49 | 57.53 | 72.38 | 78.22 | 77.34 | | |
| 0.005 | 9.81 | 24.44 | 33.78 | 35.6 | 31.8 | 52.41 | 47.51 | 65.86 | 73.08 | 71.99 | | |
| 0 | 0 | 16.22 | 26.58 | 28.6 | 24.38 | 47.23 | 41.8 | 62.15 | 70.15 | 68.94 | | |

| [DRUG] | | | | | | | | | | | SYNERGY PLOT (99%) | Cmpd 2 μM |
|---|---|---|---|---|---|---|---|---|---|---|---|---|
| Cmpd 1 μM | 0 | 0.00002 | 0.00004 | 0.00008 | 0.00016 | 0.0003 | 0.0006 | 0.001 | 0.003 | 0.005 | Bonferroni Adj. | 55% |
| 0.405 | 0 | 0 | 0 | 0 | 0 | 0 | 0 | 0 | 0 | 0 | SYNERGY | 0 |
| 0.135 | 0 | 0 | 0 | 0 | 0 | 0 | 0 | 0 | 0 | 0 | log volume | 0 |
| 0.045 | 0 | 0 | 0 | 0 | 0 | 0 | 0 | 0 | 0 | 0 | ANTAGONISM | 0 |
| 0.015 | 0 | 0 | 0 | 0 | 0 | 0 | 0 | 0 | 0 | 0 | log volume | 0 |
| 0.005 | 0 | 0 | 0 | 0 | 0 | 0 | 0 | 0 | 0 | 0 | | |
| 0 | 0 | 0 | 0 | 0 | 0 | 0 | 0 | 0 | 0 | 0 | | |

TABLE 9
Summary of results of in vitro triple combination study of Compound 1 + Compound
2 + ETV in HepG2.2.15 cell culture system with rcDNA quantitation using bDNA assay:

| | | | Avg. | | Avg. | | |
|---|---|---|---|---|---|---|---|
| | | | Synergy | Avg. | Antagonism | Avg. | |
| Inhibitor A | Inhibitor B | ETV | Volume | Synergy | Volume | Antagonism | |
| (Dose Range) | (Dose Range) | μM | (μM² %) | Log Volume | (μM² %) | Log Volume | Conclusion |
| Cmpd 1 | Cmpd 2 | 0 | 0 | 0 | −7.01 | −1.01 | Additive |
| Cmpd 1 | Cmpd 2 | 0.0003 | 0 | 0 | 0 | 0 | Additive |
| Cmpd 1 | Cmpd 2 | 0.001 | 0 | 0 | −2.23 | −0.32 | Additive |
| Cmpd 1 | Cmpd 2 | 0.003 | 0 | 0 | −1.69 | −0.24 | Additive |
| Cmpd 1 | Cmpd 2 | 0.009 | 0 | 0 | 0 | 0 | Additive |

Notes:
These values were determined at 99% confidence interval with 55% Bonferroni correction.

TABLE 10
Summary of results of in vitro triple combination study of Compound 1 + Compound
2 + TAF in HepG2.2.15 cell culture system with rcDNA quantitation using bDNA assay:

| | | | Avg. | | Avg. | | |
|---|---|---|---|---|---|---|---|
| | | | Synergy | Avg. | Antagonism | Avg. | |
| Inhibitor A | Inhibitor B | TAF | Volume | Synergy | Volume | Antagonism | |
| (Dose Range) | (Dose Range) | μM | (μM² %) | Log Volume | (μM² %) | Log Volume | Conclusion |
| Cmpd 1 | Cmpd 2 | 0 | 0 | 0 | 0 | 0 | Additive |
| Cmpd 1 | Cmpd 2 | 0.003 | 0 | 0 | −2.26 | −0.32 | Additive |
| Cmpd 1 | Cmpd 2 | 0.010 | 0 | 0 | −5.22 | −0.75 | Additive |
| Cmpd 1 | Cmpd 2 | 0.030 | 0 | 0 | −4.21 | −0.6 | Additive |
| Cmpd 1 | Cmpd 2 | 0.100 | 0 | 0 | −10.1 | −1.45 | Additive |

Notes:
These values were determined at 99% confidence interval with 55% Bonferroni correction.

TABLE 11
Summary of results of in vitro triple combination study of Compound 1 + Compound
2 + TDF in HepG2.2.15 cell culture system with rcDNA quantitation using bDNA assay:

| | | | Avg. | | Avg. | | |
|---|---|---|---|---|---|---|---|
| | | | Synergy | Avg. | Antagonism | Avg. | |
| Inhibitor A | Inhibitor B | TDF | Volume | Synergy | Volume | Antagonism | |
| (Dose Range) | (Dose Range) | μM | (μM² %) | Log Volume | (μM² %) | Log Volume | Conclusion |
| Cmpd 1 | Cmpd 2 | 0 | 0 | 0 | −1.06 | −0.15 | Additive |
| Cmpd 1 | Cmpd 2 | 0.010 | 0 | 0 | −14.15 | −2.03 | Additive |
| Cmpd 1 | Cmpd 2 | 0.030 | 0 | 0 | −18.99 | −2.73 | Additive |
| Cmpd 1 | Cmpd 2 | 0.100 | 0 | 0 | 0 | 0 | Additive |

### Example 12: Evaluation of Combination Comprising Compound (1), Compound (2) and TDF

A mouse model of hepatitis B virus (HBV) was used to assess the anti-HBV effects of a small molecule HBV RNA destabilizer and a small molecule inhibitor of HBV encapsidation, both as independent treatments, in combination with each other and in combination with an approved nucleos(t)ide analog compound.

The HBV RNA destabilizer (Compound (2)) has the following structure:

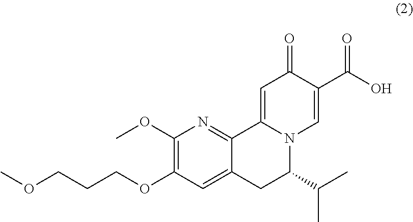 [SMILES: COCCCOc1cc2c(nc1OC)-c1cc(=O)c(C(=O)O)cn1[C@H](C(C)C)C2]

The inhibitor of HBV encapsidation (Compound (1)) has the following structure:

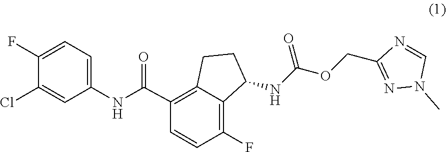 [SMILES: Cn1cnc(COC(=O)N[C@H]2CCc3c(C(=O)Nc4ccc(F)c(Cl)c4)ccc(F)c32)n1]

There are a number of nucleos(t)ide analogs approved for the treatment of chronic hepatitis B infection and their mode of action is inhibition of HBV polymerase/reverse transcriptase. In this study we specifically utilized tenofovir disproxil fumarate (TDF) as an example of this class of drug.

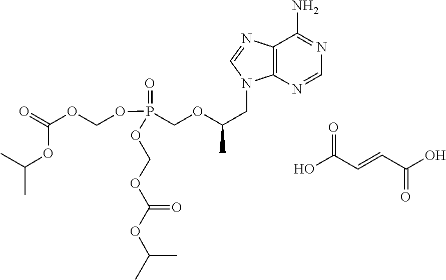 [SMILES: CC(C)OC(=O)OCOP(=O)(CO[C@H](C)Cn1cnc2c(N)ncnc21)OCOC(=O)OC(C)C ; O=C(O)/C=C/C(=O)O]

On Day −7, 10 micrograms of the plasmid pHBV1.3 (constructed based on details provided in Guidotti, L., et al., Journal of Virology, 1995, 69(10): 6158-6169) was administered to NOD.CB 17-Prkdc^{scid}/J mice via hydrodynamic injection (HDI; rapid 1.6 mL injection into the tail vein). This plasmid carries a 1.3-fold overlength copy of a HBV genome (genotype D, serotype ayw) which, when expressed, generates hepatitis B viral particles including HBV DNA and HBsAg. As readouts of the anti-HBV effect of the treatments, serum HBV DNA and serum HBsAg were assessed. Serum HBV DNA concentration in mice was measured using a quantitative PCR assay following total DNA extraction using previously published primers and probe sequences (Tanaka, Y., et al., Journal of Medical Virology, 2004, 72: 223-229). Serum HBsAg concentration in mice was measured using a commercially available ELISA kit (HBsAg EIA 3.0 480 Test Kit, Bio-Rad).

Animals were treated with RNA destabilizer as follows: Starting on Day 0, a 10 mg/kg dosage of RNA destabilizer was administered orally to animals on a once-daily frequency for a total of seven doses across the duration of the study. Animals were treated with encapsidation inhibitor as follows: Starting on Day 0, a 100 mg/kg dosage of encapsidation inhibitor was administered orally to animals on a once-daily frequency for a total of seven doses across the duration of the study. Animals were treated with nucleos(t)ide analog as follows: Starting on Day 0, a 0.4 mg/kg dosage of nucleos(t)ide analog was administered orally to animals on a once-daily frequency for a total of seven doses across the duration of the study. The RNA destabilizer, the encapsidation inhibitor, and nucleos(t)ide analog were each dissolved in the same co-solvent formulation for administration and negative control animals were administered the co-solvent formulation alone. To calculate treatment-specific effects, the treated groups are compared against negative control (vehicle treated) animals.

The effect of these treatments was determined by collecting blood on Days −1 (prior to study's treatment phase), 4, and 7 and analyzing it for serum HBV DNA and HBsAg content. Table 12 shows the treatment group mean (n=7 or 8; ±standard error of the mean) serum HBV DNA concentration expressed as a log reduction from negative control as a percentage of Day −1 baseline. Table 13 shows the treatment group mean (n=7 or 8; ±standard error of the mean) serum HBsAg concentration expressed as a log reduction from negative control as a percentage of Day −1 baseline.

The study outcomes are as follows: 1. Consistent with the understood drug mechanisms of action, the combination of treatments resulted in a greater reduction in viral replication (as represented by the serum HBV DNA biomarker) than any of the individual agents alone, and the mean reduction from the triple combination was greater than that of any of the dual combinations. 2. The reductive effect on viral protein production (as represented by the serum HBsAg biomarker) was caused by the RNA destabilizer and was not antagonized when the RNA destabilizer was administered in combination with either the capsid inhibitor or the nucleos(t)ide analog or both agents together.

TABLE 12
Serum HBV DNA reduction in a mouse model of HBV infection following once
daily oral administration of an RNA destabilizer, encapsidation inhibitor
and nucleos(t)ide analog separately and in dual and triple combination.

| | | | | Serum HBV DNA log | |
|---|---|---|---|---|---|
| | | | | reduction | |
| | Agent 1 | Agent 2 | Agent 3 | Day 4 | Day 7 |
| Group 1 | none | none | none | 0.07 ± 0.09 | 0.02 ± 0.05 |
| Group 2 | RNA | none | none | 0.48 ± 0.11 | 0.48 ± 0.10 |
| | Destabilizer | | | | |
| Group 3 | none | Encapsidation | none | 0.94 ± 0.07 | 0.86 ± 0.12 |
| | | Inhibitor | | | |
| Group 4 | none | none | Nucleos(t)ide | 1.2 ± 0.2 | 1.7 ± 0.1 |
| | | | analog | | |
| Group 5 | RNA | Encapsidation | none | 1.7 ± 0.1 | 1.4 ± 0.1 |
| | Destabilizer | Inhibitor | | | |
| Group 6 | RNA | none | Nucleos(t)ide | 1.7 ± 0.1 | 2.1 ± 0.2 |
| | Destabilizer | | analog | | |
| Group 7 | none | Encapsidation | Nucleos(t)ide | 1.7 ± 0.1 | 1.9 ± 0.1 |
| | | Inhibitor | analog | | |
| Group 8 | RNA | Encapsidation | Nucleos(t)ide | 2.5 ± 0.1 | 2.8 ± 0.1 |
| | Destabilizer | Inhibitor | analog | | |

TABLE 13
Serum HBsAg reduction in a mouse model of HBV infection following once daily
oral administration of an RNA destabilizer, encapsidation inhibitor and
nucleos(t)ide analog separately and in dual and triple combination.

| | | | | Serum HBsAg log | |
|---|---|---|---|---|---|
| | | | | reduction | |
| | Agent 1 | Agent 2 | Agent 3 | Day 4 | Day 7 |
| Group 1 | none | none | none | 0.03 ± 0.06 | 0.01 ± 0.03 |
| Group 2 | RNA | none | none | 0.75 ± 0.03 | 0.86 ± 0.04 |
| | Destabilizer | | | | |
| Group 3 | none | Encapsidation | none | −0.12 ± 0.03 | 0.07 ± 0.04 |
| | | Inhibitor | | | |
| Group 4 | none | none | Nucleos(t)ide | −0.01 ± 0.12 | 0.10 ± 0.07 |
| | | | analog | | |
| Group 5 | RNA | Encapsidation | none | 0.82 ± 0.06 | 0.90 ± 0.05 |
| | Destabilizer | Inhibitor | | | |
| Group 6 | RNA | none | Nucleos(t)ide | 0.73 ± 0.07 | 0.98 ± 0.07 |
| | Destabilizer | | analog | | |
| Group 7 | none | Encapsidation | Nucleos(t)ide | −0.01 ± 0.06 | 0.11 ± 0.04 |
| | | Inhibitor | analog | | |
| Group 8 | RNA | Encapsidation | Nucleos(t)ide | 0.78 ± 0.07 | 1.0 ± 0.1 |
| | Destabilizer | Inhibitor | analog | | |

### Examples 13-14: Evaluation of Combinations Comprising Compound (2) and ETV or Compound 2 and TAF

In vitro Combination Study Goal:

Compound (2) is a small molecule that specifically destabilizes HBV RNAs (pgRNA and sRNA). Consequently, HBV proteins, such as hepatitis B e antigen (HBeAg) and hepatitis B surface antigen (HBsAg), as well as HBV DNA replication are also inhibited by Compound (2). However, the nucleoside analog inhibitors entecavir (ETV) and tenofovir alefenamide (TAF) solely target HBV DNA replication. Therefore, the HepG2.2.15 cell line was used to determine whether two compounds (HBV RNA destabilizer and HBV DNA inhibitor) in a combination treatment would result in a synergistic, antagonistic, or additive effect in vitro.

### Small Molecule Chemical Structure:

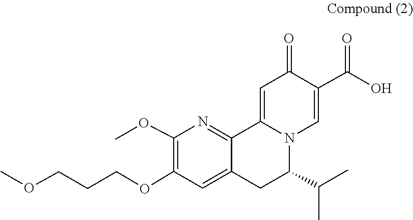 [SMILES: COCCCOc1cc2c(nc1OC)-c1cc(=O)c(C(=O)O)cn1[C@H](C(C)C)C2]

### In Vitro Combination Experimental Protocol:

In vitro combination studies were conducted using the method of Prichard and Shipman (Prichard M N, and Shipman C Jr., Antiviral Research, 1990, 14(4-5), 181-205; and Prichard M N, et. al., MacSynergy II). The HepG2.2.15 cell culture system is a cell line derived from human hepatoblastoma HepG2 cells, which have been stably transfected with the adw2-subtype HBV genome as previously explained in Sells et al. (Proc. Natl. Acad. Sci. U. S. A, 1987. Vol 84:1005-1009). HepG2.2.15 cells secrete Dane-like viral particles, produce HBV DNA, and produce the viral proteins, HBeAg and HBsAg.

For these combination studies the nucleoside analogs ETV and TAF will be referenced as Inhibitor A, while the HBV RNA destabilizer, compound (2), is referred to as Inhibitor B. EC₅₀ values of these agents are shown in Table 16. Although inhibition of HBV DNA, RNA and proteins can be determined in the presence of these inhibitors, we used the branched DNA assay due to its ability to quantitatively measure the level HBV DNA.

Detection of HBV DNA. The branched DNA assay (bDNA) was used to determine the effect of compound combinations on HBV DNA. HepG2.2.15 (10,000 cells/well) were cultured in DMEM medium plus supplements as described above. The next day, the cells were replenished with fresh medium followed by the addition of Inhibitor A and B, both were dissolved in 100% DMSO. The microtiter cell plates were incubated for a total duration of 6 days at 37° C. without replenishing media or compound. The serial dilutions spanned concentration ranges respective to the EC₅₀ value of each compound. In addition to combination testing of the compounds, both inhibitors A and B were also tested singly.

The level of bDNA present in the inhibitor-treated supernatant wells was measured using a Quantigene 2.0 bDNA assay kit (Affymetrix, Santa Clara, Calif.) with HBV specific custom probe set (genotype D ayw; DF-10739) and manufacturer's instructions after performing a proteinase K digestion in lysis. The plates were read using a Victor luminescence plate reader (PerkinElmer Model 1420 Multilabel counter) and the RLU data generated from each well was calculated as inhibition of the untreated control wells. The data was analyzed using the interpretive guidelines established by Prichard and Shipman combination model using the MacSynergy II program (Prichard M N, Shipman C Jr. Antiviral Research, 1990. Vol 14(4-5):181-205; Prichard M N, Aseltine KR, and Shipman, C. MacSynergy II. University of Michigan 1992) to determine whether the combinations were synergistic, additive or antagonistic using the interpretive guidelines established by Prichard and Shipman as follows: synergy volumes <25 μM2% (log volume <2) at 95% CI=probably insignificant; 25-50 (log volume >2 and <5)=minor but significant 50-100 (log volume >5 and <9)=moderate, may be important in vivo; Over 100 (log volume >9)=strong synergy, probably important in vivo; volumes approaching 1000 (log volume >90)=unusually high, check data. The RLU data from the single compound treated cells were analyzed using XL-Fit module in Microsoft Excel to determine EC₅₀ values using a 4-parameter curve fitting algorithm.

### Example 13: In Vitro Combination of Compound (2) and ETV

ETV (concentration range of 0.1 μM to 0.000015 μM in a half-log, 3.16-fold dilution series and 9-point titration) was tested in combination with Compound (2) (concentration range of 0.01 uM to 0.0001 uM in a half-log, 3.16-fold dilution series and 5-point titration). The combination results were completed in duplicate with each assay consisting of 4 technical repeats. The measurements of synergy and antagonism volumes according to Prichard and Shipman, and interpretation, are shown in Table 16. The antiviral activity of this combination is shown in Table 14a; synergy and antagonism volumes are shown in Table 14b. The synergistic activity of this combination is shown in Table 14d. In this assay system, the combination results in moderate synergy inhibition of HBV bDNA. No significant inhibition of cell viability or proliferation was observed by microscopy or Cell-Titer Glo assay (Table 14c).

TABLE 14a
Antiviral Activity of Compound (2) and ETV Combination: Average percent
inhibition versus negative control (n = 4 samples per data point)

| | | | | | | | | | | | |
|---|---|---|---|---|---|---|---|---|---|---|---|
| Cmpd 2, μM | 0.01 | 90.03 | 90.35 | 87.97 | 88.64 | 90.02 | 92.75 | 93.78 | 94.61 | 94.73 | 95.29 |
| Avg % | 0.0032 | 87.27 | 87.49 | 82.58 | 80.9 | 84.12 | 86.19 | 88.42 | 92.65 | 92.22 | 93.62 |
| Inhibition | 0.001 | 76.67 | 77.47 | 76.81 | 76.35 | 74.25 | 76.92 | 84.66 | 87.8 | 92.6 | 93.4 |
| | 0.0003 | 67.72 | 61.25 | 61.44 | 51.02 | 42.83 | 62.48 | 75.13 | 84.07 | 88.56 | 91.64 |
| | 0.0001 | 43.43 | 36.69 | 28.86 | 9.4 | 29.13 | 46.56 | 68.28 | 79.8 | 86.26 | 90.63 |
| | 0 | 0 | −90.87 | −90.2 | −88.53 | −55.97 | −21.2 | 30.18 | 48.98 | 68.14 | 85.09 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Cmpd | ETV, μM |

TABLE 14b
MacSynergy Volume Calculations of Compound (2) and ETV Combination: 99.99% confidence interval (Bonferroni Adj. 96%)

| | | | | | | | | | | | |
|---|---|---|---|---|---|---|---|---|---|---|---|
| Cmpd 2, μM | 0.01 | 0.00 | 1.32 | 0.00 | 0.00 | 0.00 | 1.70 | 0.00 | 0.00 | 0.00 | 0.16 |
| SYNERGY 43.44 | 0.0032 | 0.00 | 5.44 | 0.00 | 0.00 | 0.09 | 0.00 | 0.00 | 0.00 | −0.39 | 0.43 |
| Log volume 10.85 | 0.001 | 0.00 | 12.03 | 11.34 | 5.52 | 0.00 | 0.00 | 0.00 | 0.00 | 0.00 | 1.84 |
| Antagonism −2.82 | 0.0003 | 0.00 | 0.00 | 0.00 | 0.00 | 0.00 | 0.00 | 0.00 | 0.00 | 0.00 | 0.00 |
| Log volume −0.7 | 0.0001 | 0.00 | 6.00 | 0.00 | 0.00 | 0.00 | 0.00 | 0.00 | 0.00 | 0.00 | 0.00 |
| | 0 | 0.00 | 0.00 | 0.00 | 0.00 | 0.00 | 0.00 | 0.00 | 0.00 | 0.00 | 0.00 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | ETV, μM |

TABLE 14c
Cytotoxicity of Compound (2) and ETV Combination: Average percent of cell viability vs control

| | | | | | | | | | | | |
|---|---|---|---|---|---|---|---|---|---|---|---|
| Cmpd 2, μM | 0.01 | 92.68 | 97.37 | 100.87 | 101.54 | 102.90 | 104.04 | 104.35 | 104.4 | 92.19 | 93.66 |
| Avg % Cell | 0.0032 | 81.35 | 94.71 | 83.31 | 86.56 | 87.45 | 88.73 | 90.63 | 93.78 | 99.26 | 75.65 |
| Viability | 0.001 | 91.36 | 93.83 | 85.44 | 81.84 | 82.58 | 78.96 | 86.99 | 86.58 | 97.60 | 85.18 |
| | 0.0003 | 92.57 | 96.55 | 86.21 | 86.38 | 86.64 | 89.07 | 89.03 | 94.27 | 101.78 | 86.59 |
| | 0.0001 | 80.37 | 105.13 | 96.15 | 93.08 | 89.45 | 92.91 | 96.20 | 99.07 | 105.54 | 75.25 |
| | 0 | 100.0 | 92.37 | 101.72 | 101.37 | 101.92 | 103.37 | 104.67 | 103.6 | 91.50 | 88.20 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | ETV, μM |

TABLE 14d
Antiviral Activity of Compound (2) and ETV Combination: Additive percent
inhibition versus negative control (n = 4 samples per data point)

| | | | | | | | | | | | |
|---|---|---|---|---|---|---|---|---|---|---|---|
| Cmpd, μM | 0.01 | 90.03 | 80.97 | 81.04 | 81.2 | 84.45 | 87.92 | 93.04 | 94.91 | 96.82 | 98.51 |
| Additive % | 0.0032 | 87.27 | 75.7 | 75.79 | 76 | 80.15 | 84.57 | 91.11 | 93.51 | 95.94 | 98.1 |
| Inhibition | 0.001 | 76.67 | 55.47 | 55.63 | 56.02 | 63.61 | 71.72 | 83.71 | 88.1 | 92.57 | 96.52 |
| | 0.0003 | 67.72 | 38.39 | 38.6 | 39.14 | 49.65 | 60.88 | 77.46 | 83.53 | 89.72 | 95.19 |
| | 0.0001 | 43.43 | −7.98 | −7.6 | −6.65 | 11.77 | 31.44 | 60.5 | 71.14 | 81.98 | 91.57 |
| | 0 | 0 | −90.87 | −90.2 | −88.53 | −55.97 | −21.2 | 30.18 | 48.98 | 68.14 | 85.09 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | ETV, μM |

### Example 14: In Vitro Combination of Compound (2) and TAF

Compound (2) (concentration range of 0.01 μM to 0.000015 μM in a half-log, 3.16-fold dilution series and 5-point titration) was tested in combination with TAF (concentration range of 2.0 uM to 0.0002 uM in a half-log, 3.16-fold dilution series and 9-point titration). The combination results were completed in duplicate with each assay consisting of 4 technical repeats. The measurements of synergy and antagonism volumes according to Prichard and Shipman, and interpretation, are shown in Table 16. The antiviral activity of this combination is shown in Table 15a; synergy and antagonism volumes are shown in Table 15b. The additive inhibition activity of this combination is shown in Table 15d. In this assay system, the combination results in additive inhibition of HBV DNA. No significant inhibition of cell viability or proliferation was observed by microscopy or Cell-Titer Glo assay (Table 15c).

TABLE 15a
Antiviral Activity of Compound (2) and TAF Combination: Average percent
inhibition versus negative control (n = 4 samples per data point)

| | | | | | | | | | | | |
|---|---|---|---|---|---|---|---|---|---|---|---|
| Cmpd 2, μM | 0.01 | 89.39 | 90.15 | 89.65 | 89.66 | 89.34 | 85.29 | 91.14 | 91.27 | 91.86 | 88.55 |
| Avg % | 0.0032 | 85.5 | 86.22 | 84.47 | 82.42 | 84.26 | 85.09 | 87.44 | 89.59 | 90.87 | 93.12 |
| Inhibition | 0.001 | 77.88 | 76.36 | 70.47 | 74.57 | 68.5 | 68.86 | 78.96 | 85.11 | 87.36 | 91.67 |
| | 0.0003 | 66.75 | 69.67 | 64.97 | 68.46 | 61.54 | 68.09 | 72.2 | 73.94 | 84.34 | 90.25 |
| | 0.0001 | 58.18 | 48.96 | 31.31 | 16.76 | 6.76 | 19.45 | 52.42 | 38.49 | 76.8 | 89.45 |
| | 0 | 0 | 8.88 | 12.91 | 14.28 | 19.87 | 25.36 | 42.96 | 62.7 | 76.45 | 87.21 |
| | | 0 | 0.0002 | 0.0006 | 0.0020 | 0.0064 | 0.0201 | 0.0634 | 0.2002 | 0.6329 | 2.0 |

| | |
|---|---|
| Cmpd | TAF, μM |

TABLE 15b
MacSynergy Volume Calculations of Compound (2) and TAF Combination:
99.99% confidence interval (Bonferroni Adj. 96%)

| | | | | | | | | | | | |
|---|---|---|---|---|---|---|---|---|---|---|---|
| Cmpd 2, μM | 0.01 | 0 | 0 | 0 | 0 | 0 | 0 | 0 | 0 | 0 | 0 |
| SYNERGY 0 | 0.0032 | 0 | 0 | 0 | 0 | 0 | 0 | 0 | 0 | 0 | 0 |
| Log volume 0 | 0.001 | 0 | 0 | 0 | 0 | 0 | 0 | 0 | 0 | 0 | 0 |
| Antagonism 0 | 0.0003 | 0 | 0 | 0 | 0 | 0 | 0 | 0 | 0 | 0 | 0 |
| Log volume 0 | 0.0001 | 0 | 0 | 0 | 0 | 0 | 0 | 0 | 0 | 0 | 0 |
| | 0 | 0 | 0 | 0 | 0 | 0 | 0 | 0 | 0 | 0 | 0 |
| | | 0 | 0.0002 | 0.0006 | 0.0020 | 0.0064 | 0.0201 | 0.0634 | 0.2002 | 0.6329 | 2.0 |

| | |
|---|---|
| Compound | TAF, μM |

TABLE 15c
Cytotoxicity of Compound (2) and TAF Combination: Average percent of cell viability vs control

| | | | | | | | | | | | |
|---|---|---|---|---|---|---|---|---|---|---|---|
| Cmpd 2, μM | 0.01 | 104.89 | 106.56 | 109.10 | 105.05 | 105.57 | 106.99 | 102.17 | 92.41 | 82.68 | 100.79 |
| Avg % | 0.0032 | 104.08 | 108.12 | 106.63 | 105.95 | 103.34 | 107.32 | 96.22 | 64.90 | 68.85 | 82.97 |
| Cell | 0.001 | 103.79 | 105.29 | 106.97 | 104.72 | 103.14 | 105.83 | 96.23 | 78.45 | 67.75 | 80.13 |
| Viability | 0.0003 | 101.80 | 104.33 | 105.90 | 105.96 | 102.53 | 106.00 | 98.96 | 54.42 | 56.43 | 83.94 |
| | 0.0001 | 99.27 | 104.62 | 105.93 | 104.12 | 102.46 | 105.20 | 98.60 | 71.30 | 59.08 | 85.71 |
| | 0 | 100.00 | 103.95 | 104.32 | 103.73 | 101.18 | 101.33 | 103.56 | 96.48 | 80.67 | 88.67 |
| | | 0 | 0.0002 | 0.0006 | 0.0020 | 0.0064 | 0.0201 | 0.0634 | 0.2002 | 0.6329 | 2.0 |

| | |
|---|---|
| Cmpd | TAF, μM |

TABLE 15d
Antiviral Activity of Compound (2) and TAF Combination: Additive percent
inhibition versus negative control (n = 4 samples per data point)

| | | | | | | | | | | | |
|---|---|---|---|---|---|---|---|---|---|---|---|
| Cpmd (2), μM | 0.01 | 89.39 | 90.33 | 90.76 | 90.91 | 91.5 | 92.08 | 93.95 | 96.04 | 97.5 | 98.64 |
| Additive % | 0.0032 | 85.5 | 86.79 | 87.37 | 87.57 | 88.38 | 89.18 | 91.73 | 94.59 | 96.59 | 98.15 |
| Inhibition | 0.001 | 77.88 | 79.84 | 80.74 | 81.04 | 82.28 | 83.49 | 87.38 | 91.75 | 94.79 | 97.17 |
| | 0.0003 | 66.75 | 69.7 | 71.04 | 71.5 | 73.36 | 75.18 | 81.03 | 87.6 | 92.17 | 95.75 |
| | 0.0001 | 58.18 | 61.89 | 63.58 | 64.15 | 66.49 | 68.79 | 76.15 | 84.4 | 90.15 | 94.65 |
| | 0 | 0 | 8.88 | 12.91 | 14.28 | 19.87 | 25.36 | 42.96 | 62.7 | 76.45 | 87.21 |
| | | 0 | 0.0002 | 0.0006 | 0.0020 | 0.0064 | 0.0201 | 0.0634 | 0.2002 | 0.6329 | 2.0 |

| | |
|---|---|
| Compound | TAF, μM |

TABLE 16
Summary of results of in vitro combination studies in HepG2.2.15 cell culture system with bDNA quantitation

| | | | Inhibitor | Inhibitor | Synergy | Synergy | Antagonism | | |
|---|---|---|---|---|---|---|---|---|---|
| Example | Inhibitor | Inhibitor | A EC₅₀ | B EC₅₀ | Volume | Log | Volume | Antagonism | |
| Number | A | B | (μM) | (μM) | (μM² %) | Volume | (μM² %)* | Log Volume | Interpretation |
| 5 | ETV | Cmpd 2 | 0.0032 | 0.0009 | 74.36 | 18.56 | −0.94 | −0.23 | Moderate |
| | | | | | | | | | Synergy |
| 6 | TAF | Cmpd 2 | 0.054 | 0.0006 | 11.67 | 2.91 | −12.9 | −3.22 | Additive |

*at 99.9% confidence interval

### Examples 15-17

For the Examples 15-17 below, a compound of Formula (I), wherein the siRNA is siRNA 2 as described (Compound 1) was prepared using procedures similar to those described in International Patent Application Publication Number WO2018/191278. Entecavir was purchased from Bide Pharmatech Ltd. (Catalog Number BD127328WG0127328-160902001). Tenofovir disoproxil fumarate was purchased from Shanghai Titan Scientific Co., Ltd (Catalog Number P1131909)

In certain embodiments, the siRNA of the siRNA conjugate is siRNA 1 below. In certain embodiments, the siRNA of the siRNA conjugate is siRNA 2 below. In the experiments described hereinbelow, the siRNA of the siRNA conjugate is siRNA 2 below. The compound of formula (1) is depicted below, wherein the siRNA of the siRNA conjugate is siRNA 2.

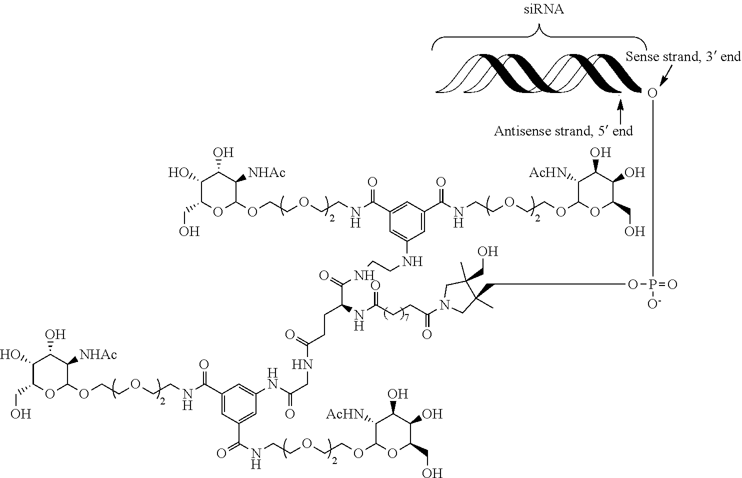

| siRNA Name | Sense Sequence (5′-3′) | Antisense Sequence (5′- 3′) |
|---|---|---|
| siRNA 1 | usgscaCUUcgcuucaccu | asGsgugaagcgaagUgCacascsgU |
| siRNA 2 | gsusgcACUucgcuucaca | usGsugaagcgaaguGcAcacsgsgU |

2′-O-Methyl nucleotides = lower case
2′-Fluoro nucleotides = UPPER_CASE
Phosphorothioate linker = s
Unmodified = UPPER CASE

### Example 15: Combination Studies in Primary Human Hepatocytes PHHs

Cryopreserved PHHs (Lot QBU) were purchased from Bioreclamation IVT

### Infectious Virus Stock

Genotype D HBV was concentrated from HepG2DE19 culture supernatants. Information on the infectious virus stock is shown in the following Table.

| | | HBV titer | | |
|---|---|---|---|---|
| | | in serum | HBV | |
| Virus ID | Lot# | (GE*/ml) | Genotype | Source |
| HBV-DE19 | 20180313 | 3.2E+10 GE/ml | D | HepDE19 |
| | | | | supernatants |

*GE = HBV genome equivalent.

### Reagents

The major reagents used in the study were QIAamp 96 DNA Blood Kit (QIAGEN #51162), FastStart Universal Probe Master (Roche #04914058001), CellTiter-Glo (Promega #G7573) and HBsAg ELISA kit (Antu #CL 0310), and Lipofectamine 3000 Transfection Kit (invitrogen #L3000-015).

### Instruments

The major instruments used in the study were BioTek Synergy 2, SpectraMax (Molecular Devices), and 7900HT Fast Real-Time PCR System (ABI).

### Seeding of Primary Human Hepatocytes

The PHH were thawed and seeded into 48-well plates at a density of 1.32×10⁵ cells/well. The day PHH seeding date was defined as day 0.

### HBV Infection

The PHH were infected with 400 HBV GE/cell of D type HBV on day 1.

### Culture and Treatment of PHHs.

On day 0, 6-8 hours after cell seeding, compound 1 was serially diluted in a 3-fold dilution series with media containing the transfection reagent to make 26.55× (for single compound dose response study) or 265.5× (for double combination studies) of the final test concentrations. The test articles were further diluted with the culture medium to the final test concentrations.

On day 2, the test articles TDF and ETV were serially diluted with DMSO to make 100× of the final test concentrations. All the test articles were further diluted 100 times with the culture medium. The final concentration of DMSO in the culture medium was 2%.

### Determination of EC₅₀ Values.

Compound 1, ETV, and TDF were tested at 6 or 7 concentrations, in a 3-fold dilution series, in triplicate samples.

### Double Combination Study.

Four two-way combinations were performed on a 5×5 matrix, in triplicate plates. Transfection reagent was present in all wells. Compound 1 was transfected only once, at day 0, and the culture medium containing DMSO, ETV or TDF were refreshed every 1 or 2 days.

### Assay for Cytotoxicity by CellTiter Glo Assay at Day 8

One day 8, the culture supernatants were collected, and CellTiter-Glo working solution was added to the cell plates. The plates were incubated at room temperature 10 mins. The lysates were transferred into a 96-well black plate. Luminescence signal was measured on a BioTek Synergy 2 SpectraMax. Percent cell viability was calculated with the formula below:

Viability %=(raw data of sample−AVG. of blank)/(AVG. of Medium control−AVG. of blank)×100

Quantification of HBV DNA in the Culture Supernatants by qPCR

DNA in the culture supernatants harvested on days 8 was isolated with QIAamp 96 DNA Blood Kit (Qiagen-51162). For each sample, 100 μl of each culture supernatant was used to extract DNA. The DNA was eluted with 180 μl of AE. HBV DNA in the culture supernatants was quantified by quatitative PCR using well-established and commonly used procedures. Percent inhibition of HBV DNA was calculated with the formula below:

% Inh. HBV DNA=[1−value of sample/AVG. value of Medium control]×100.

### Measurement of HBsAg in the Culture Supernatants by ELISA

HBsAg in the culture supernatants harvested on days 8 was measured using the HBsAg/ELISA kit (Autobio) according to the manual. The samples were diluted 4-fold with PBS to get the signal in the range of the standard curve. Percent inhibition of HBsAg was calculated with the following formula:

% Inh. HBsAg=[1−HBsAg quantity of sample/HBV quantity of DMSO control]×100

### Analysis of Combination Effects

Results of double combination studies were analyzed using MacSynergy II software (Prichard and Shipman, 1992). Combination effects were calculated as synergy/antagonism volumes to 99.9% confidence interval, and results were interpreted according to MacSynergy II guidelines, as follows:

<25=Insignificant synergism/antagonism

25-50=Minor but significant synergism/antagonism

50-100=Moderate synergism/antagonism—may be important in vivo

>100=Strong synergism/antagonism—probably important in vivo

### Example 16: In Vitro Combination of Compound 1 and ETV

### Study Goal:

To determine whether a two-drug combination of compound 1 and entecavir (ETV) is additive, synergistic or antagonistic in vitro, using HBV-infected human primary hepatocytes in a cell culture model system.

### Results and Conclusion:

ETV (concentration range of 0.07 nM to 0.00086 nM in a 3-fold dilution series and 5 point titration) was tested in combination with compound 1 (concentration range of 1.0 ng/mL to 0.012 ng/mL in a 3-fold dilution series and 5 point titration), on three replicate plates in each of two separate experimental trials The average % inhibition in HBV DNA and HBsAg, and standard deviations of 3 replicates observed either with ETV or the compound of formula (I) treatments alone or in combination are shown in Tables 2A, 2B, 2C, and 2D as indicated below. The EC₅₀ values of ETV and compound 1 were determined in an earlier experiment and are shown in Table 3.

When the observed values of a two-inhibitor combination were compared to what is expected from additive interaction for the above concentration range, the combination effects ranged from additive for HBV DNA inhibition, with no significant synergy or antagonism, to synergistic for HBsAg inhibition, as per MacSynergy II analysis, and using the interpretive criteria described by Prichard and Shipman (1992) (Table 2E). No significant inhibition of cell viability was observed by microscopy or CellTiter-Glo assay.

TABLE 2A
Effect on HBV DNA in In Vitro Combination of Compound 1 and ETV

| [DRUG] | | | | | | | AVERAGE % INHIBITION |
|---|---|---|---|---|---|---|---|
| ETV | | | | | | | Horizontal axis: |
| nM | 0.00 | 0.01 | 0.037 | 0.11 | 0.33 | 1.00 | Compound 1 (μg/mL) |
| 0.07000 | 91.94 | 92.94 | 93.27 | 94.54 | 93.79 | 92.84 | |
| 0.02300 | 81.68 | 86.19 | 87.9 | 89.98 | 88.93 | 90.88 | |
| 0.00780 | 51.34 | 66.69 | 73.55 | 79.55 | 86.52 | 85.33 | |
| 0.00260 | 21.99 | 50.21 | 61.69 | 69.58 | 78.17 | 80.38 | |
| 0.00086 | 17.71 | 44.06 | 58.04 | 66.6 | 72.48 | 75.72 | |
| 0.00000 | 0 | 41.6 | 49.56 | 64.02 | 69.24 | 76.83 | |

| [DRUG] | | | | | | | STANDARD DEVIATION (%) |
|---|---|---|---|---|---|---|---|
| ETV | | | | | | | Horizontal axis: |
| nM | 0.00 | 0.01 | 0.037 | 0.11 | 0.33 | 1.00 | Compound 1 (μg/mL) |
| 0.07000 | 1.49 | 0.82 | 0.63 | 0.39 | 0.78 | 0.84 | |
| 0.02300 | 2.1 | 2.95 | 1.66 | 2.92 | 1.64 | 1.28 | |
| 0.00780 | 7.36 | 4.46 | 4.73 | 2.66 | 0.53 | 2.05 | |
| 0.00260 | 17.38 | 7.24 | 1.61 | 2.62 | 2.84 | 1.86 | |
| 0.00086 | 6.97 | 11.22 | 5.41 | 6.1 | 4.45 | 2.66 | |
| 0.00000 | 13.07 | 15.66 | 3.85 | 5.95 | 5.08 | 2.05 | |

| [DRUG] | | | | | | | ADDITIVE % INHIBITION |
|---|---|---|---|---|---|---|---|
| ETV | | | | | | | Horizontal axis: |
| nM | 0.00 | 0.01 | 0.037 | 0.11 | 0.33 | 1.00 | Compound 1 (μg/mL) |
| 0.07000 | 91.94 | 95.29 | 95.93 | 97.1 | 97.52 | 98.13 | |
| 0.02300 | 81.68 | 89.3 | 90.76 | 93.41 | 94.36 | 95.76 | |
| 0.00780 | 51.34 | 71.58 | 75.46 | 82.49 | 85.03 | 88.73 | |
| 0.00260 | 21.99 | 54.44 | 60.65 | 71.93 | 76 | 81.93 | |
| 0.00086 | 17.71 | 51.94 | 58.49 | 70.39 | 74.69 | 80.93 | |
| 0.00000 | 0 | 41.6 | 49.56 | 64.02 | 69.24 | 76.83 | |

| [DRUG] | | | | | | | SYNERGY PLOT (99.9%) |
|---|---|---|---|---|---|---|---|
| ETV | | | | | | | Bonferroni Adj. |
| nM | 0.00 | 0.01 | 0.037 | 0.11 | 0.33 | 1.00 | 98% |
| 0.07000 | 0 | 0 | −0.58667 | −1.27651 | −1.16302 | −2.52556 | SYNERGY 0 |
| 0.02300 | 0 | 0 | 0 | 0 | −0.03276 | −0.66752 | log volume 0 |
| 0.00780 | 0 | 0 | 0 | 0 | 0 | 0 | |
| 0.00260 | 0 | 0 | 0 | 0 | 0 | 0 | ANTAGONISM −6.25 |
| 0.00086 | 0 | 0 | 0 | 0 | 0 | 0 | log volume −1.42 |
| 0.00000 | 0 | 0 | 0 | 0 | 0 | 0 | |

TABLE 2B
Effect on HBV DNA in In Vitro Combination of Compound 1 and ETV

| [DRUG] | | | | | | | AVERAGE % INHIBITION |
|---|---|---|---|---|---|---|---|
| ETV | | | | | | | Horizontal axis: |
| nM | 0.00 | 0.01 | 0.037 | 0.11 | 0.33 | 1.00 | Compound 1 (μg/mL) |
| 0.07000 | 85.66 | 88.62 | 89.75 | 89.83 | 90.3 | 89.47 | |
| 0.02300 | 62.29 | 74.3 | 78.78 | 82.95 | 81.7 | 84.14 | |
| 0.00780 | 24.18 | 52.28 | 65.17 | 68.52 | 77.15 | 80.12 | |
| 0.00260 | 8.16 | 44.27 | 54.6 | 64.2 | 67.75 | 77.16 | |
| 0.00086 | −5.71 | 30.41 | 46.3 | 59.68 | 67.27 | 71.25 | |
| 0.00000 | 0 | 27.5 | 39.51 | 48.66 | 57.26 | 67.14 | |

| [DRUG] | | | | | | | STANDARD DEVIATION (%) |
|---|---|---|---|---|---|---|---|
| ETV | | | | | | | Horizontal axis: |
| nM | 0.00 | 0.01 | 0.037 | 0.11 | 0.33 | 1.00 | Compound 1 (μg/mL) |
| 0.07000 | 0.95 | 1.84 | 2.96 | 2.89 | 1.21 | 0.31 | |
| 0.02300 | 6.11 | 0.59 | 1.18 | 1.11 | 2.16 | 2.1 | |
| 0.00780 | 5.37 | 4.83 | 1.77 | 3.33 | 3.06 | 2.67 | |
| 0.00260 | 6.54 | 7.31 | 3.7 | 6.53 | 4.85 | 2.98 | |
| 0.00086 | 24.91 | 7.15 | 10.06 | 9.55 | 4.34 | 5.53 | |
| 0.00000 | 28.17 | 1.69 | 5.61 | 10.78 | 8.21 | 10 | |

| [DRUG] | | | | | | | ADDITIVE % INHIBITION |
|---|---|---|---|---|---|---|---|
| ETV | | | | | | | Horizontal axis: |
| nM | 0.00 | 0.01 | 0.037 | 0.11 | 0.33 | 1.00 | Compound 1 (μg/mL) |
| 0.07000 | 85.66 | 89.6 | 91.33 | 92.64 | 93.87 | 95.29 | |
| 0.02300 | 62.29 | 72.66 | 77.19 | 80.64 | 83.88 | 87.61 | |
| 0.00780 | 24.18 | 45.03 | 54.14 | 61.07 | 67.59 | 75.09 | |
| 0.00260 | 8.16 | 33.42 | 44.45 | 52.85 | 60.75 | 69.82 | |
| 0.00086 | −5.71 | 23.36 | 36.06 | 45.73 | 54.82 | 65.26 | |
| 0.00000 | 0 | 27.5 | 39.51 | 48.66 | 57.26 | 67.14 | |

| [DRUG] | | | | | | | SYNERGY PLOT (99.9%) |
|---|---|---|---|---|---|---|---|
| ETV | | | | | | | Bonferroni Adj. |
| nM | 0.00 | 0.01 | 0.037 | 0.11 | 0.33 | 1.00 | 98% |
| 0.07000 | 0 | 0 | 0 | 0 | 0 | −4.79979 | SYNERGY 5.20 |
| 0.02300 | 0 | 0 | 0 | 0 | 0 | 0 | log volume 1.18 |
| 0.00780 | 0 | 0 | 5.20493 | 0 | 0 | 0 | ANTAGONISM −4.80 |
| 0.00260 | 0 | 0 | 0 | 0 | 0 | 0 | log volume −1.09 |
| 0.00086 | 0 | 0 | 0 | 0 | 0 | 0 | |
| 0.00000 | 0 | 0 | 0 | 0 | 0 | 0 | |

TABLE 2C
Effect on HBsAg in In Vitro Combination of Compound 1 and ETV

| [DRUG] | | | | | | | AVERAGE % INHIBITION |
|---|---|---|---|---|---|---|---|
| ETV | | | | | | | Horizontal axis: |
| nM | 0.00 | 0.01 | 0.037 | 0.11 | 0.33 | 1.00 | Compound 1 (μg/mL) |
| 0.07000 | 2.91 | 44.9 | 63.07 | 74.99 | 82.83 | 88.95 | |
| 0.02300 | 0.19 | 53.85 | 64.57 | 78.49 | 85.39 | 90.48 | |
| 0.00780 | −0.05 | 51.68 | 67.27 | 79.11 | 86.5 | 90.56 | |
| 0.00260 | 4.92 | 53.34 | 65.48 | 79.04 | 85.99 | 90.68 | |
| 0.00086 | 3.04 | 51.33 | 64.11 | 77.86 | 85.67 | 90.69 | |
| 0.00000 | 0 | 40.16 | 57.6 | 72.92 | 81.93 | 88.47 | |

| [DRUG] | | | | | | | STANDARD DEVIATION (%) |
|---|---|---|---|---|---|---|---|
| ETV | | | | | | | Horizontal axis: |
| nM | 0.00 | 0.01 | 0.037 | 0.11 | 0.33 | 1.00 | Compound 1 (μg/mL) |
| 0.07000 | 2.51 | 3.94 | 2.78 | 2.64 | 0.39 | 0.63 | |
| 0.02300 | 2.52 | 0.89 | 3.42 | 1.71 | 1.1 | 0.97 | |
| 0.00780 | 5.11 | 2.45 | 1.58 | 0.53 | 0.6 | 0.12 | |
| 0.00260 | 2.62 | 0.59 | 1.11 | 0.59 | 1.18 | 0.53 | |
| 0.00086 | 5.77 | 1.73 | 2.71 | 0.91 | 0.92 | 0.66 | |
| 0.00000 | 7.17 | 9.23 | 2.17 | 2.93 | 0.8 | 0.35 | |

| [DRUG] | | | | | | | ADDITIVE % INHIBITION |
|---|---|---|---|---|---|---|---|
| ETV | | | | | | | Horizontal axis: |
| nM | 0.00 | 0.01 | 0.037 | 0.11 | 0.33 | 1.00 | Compound 1 (μg/mL) |
| 0.07000 | 2.91 | 41.9 | 58.83 | 73.71 | 82.46 | 88.81 | |
| 0.02300 | 0.19 | 40.27 | 57.68 | 72.97 | 81.96 | 88.49 | |
| 0.00780 | −0.05 | 40.13 | 57.58 | 72.91 | 81.92 | 88.46 | |
| 0.00260 | 4.92 | 43.1 | 59.69 | 74.25 | 82.82 | 89.04 | |
| 0.00086 | 3.04 | 41.98 | 58.89 | 73.74 | 82.48 | 88.82 | |
| 0.00000 | 0 | 40.16 | 57.6 | 72.92 | 81.93 | 88.47 | |

| [DRUG] | | | | | | | SYNERGY PLOT (99.9%) |
|---|---|---|---|---|---|---|---|
| ETV | | | | | | | Bonferroni Adj. |
| nM | 0.00 | 0.01 | 0.037 | 0.11 | 0.33 | 1.00 | 98% |
| 0.07000 | 0 | 0 | 0 | 0 | 0 | 0 | SYNERGY 45.62 |
| 0.02300 | 0 | 10.65101 | 0 | 0 | 0 | 0 | log volume 10.39 |
| 0.00780 | 0 | 3.48705 | 4.49022 | 4.45577 | 2.6054 | 1.70508 | ANTAGONISM 0 |
| 0.00260 | 0 | 8.29831 | 2.13699 | 2.84831 | 0 | 0 | log volume 0 |
| 0.00086 | 0 | 3.65657 | 0 | 1.12519 | 0.16228 | 0 | |
| 0.00000 | 0 | 0 | 0 | 0 | 0 | 0 | |

TABLE 2D
Effect on HBsAg in In Vitro Combination of Compound 1 and ETV

| [DRUG] | | | | | | | AVERAGE % INHIBITION |
|---|---|---|---|---|---|---|---|
| ETV | | | | | | | Horizontal axis: |
| nM | 0.00 | 0.01 | 0.037 | 0.11 | 0.33 | 1.00 | Compound 1 (μg/mL) |
| 0.07000 | −16.23 | 44.29 | 60.78 | 75.69 | 84.9 | 90.09 | |
| 0.02300 | −6.78 | 53.44 | 67.69 | 79.96 | 87.27 | 91.76 | |
| 0.00780 | −13.02 | 54.66 | 69.64 | 80.6 | 87.75 | 92.15 | |
| 0.00260 | −1.45 | 53.53 | 66.69 | 80.6 | 87.22 | 91.56 | |
| 0.00086 | −11.39 | 50.76 | 66.15 | 78.64 | 86.3 | 91.06 | |
| 0.00000 | 0 | 41.87 | 54.91 | 70.79 | 81.3 | 87.14 | |

| [DRUG] | | | | | | | STANDARD DEVIATION (%) |
|---|---|---|---|---|---|---|---|
| ETV | | | | | | | Horizontal axis: |
| nM | 0.00 | 0.01 | 0.037 | 0.11 | 0.33 | 1.00 | Compound 1 (μg/mL) |
| 0.07000 | 3.61 | 3.09 | 3.28 | 1.45 | 1.37 | 0.82 | |
| 0.02300 | 9.74 | 2.29 | 0.81 | 1.92 | 0.46 | 0.6 | |
| 0.00780 | 5.23 | 2.87 | 2.12 | 0.84 | 0.85 | 0.46 | |
| 0.00260 | 3.84 | 2.7 | 1.68 | 0.24 | 0.19 | 0.49 | |
| 0.00086 | 4.06 | 3.34 | 2.26 | 2.04 | 1.01 | 0.66 | |
| 0.00000 | 8.74 | 3.46 | 0.4 | 2.77 | 0.92 | 0.47 | |

| [DRUG] | | | | | | | ADDITIVE % INHIBITION |
|---|---|---|---|---|---|---|---|
| ETV | | | | | | | Horizontal axis: |
| nM | 0.00 | 0.01 | 0.037 | 0.11 | 0.33 | 1.00 | Compound 1 (μg/mL) |
| 0.07000 | −16.23 | 32.44 | 47.59 | 66.05 | 78.26 | 85.05 | |
| 0.02300 | −6.78 | 37.93 | 51.85 | 68.81 | 80.03 | 86.27 | |
| 0.00780 | −13.02 | 34.3 | 49.04 | 66.99 | 78.87 | 85.47 | |
| 0.00260 | −1.45 | 41.03 | 54.26 | 70.37 | 81.03 | 86.95 | |
| 0.00086 | −11.39 | 35.25 | 49.77 | 67.46 | 79.17 | 85.68 | |
| 0.00000 | 0 | 41.87 | 54.91 | 70.79 | 81.3 | 87.14 | |

| [DRUG] | | | | | | | SYNERGY PLOT (99.9%) |
|---|---|---|---|---|---|---|---|
| ETV | | | | | | | Bonferroni Adj. |
| nM | 0.00 | 0.01 | 0.037 | 0.11 | 0.33 | 1.00 | 98% |
| 0.07000 | 0 | 1.68081 | 2.39552 | 4.86805 | 2.13133 | 2.34138 | SYNERGY 148.73 |
| 0.02300 | 0 | 7.97361 | 13.17429 | 4.83128 | 5.72614 | 3.5154 | log volume 33.86 |
| 0.00780 | 0 | 10.91483 | 13.62308 | 10.84556 | 6.08265 | 5.16614 | ANTAGONISM 0 |
| 0.00260 | 0 | 3.6143 | 6.90112 | 9.44016 | 5.56471 | 2.99741 | log volume 0 |
| 0.00086 | 0 | 4.51806 | 8.94234 | 4.46636 | 3.80609 | 3.20794 | |
| 0.00000 | 0 | 0 | 0 | 0 | 0 | 0 | |

TABLE 2E
Summary of results of in vitro combination studies of Compound 1 and ETV in PHH cell culture system

| HBV | ETV | Cmpd 1 | | Synergy | | Antagonism | |
|---|---|---|---|---|---|---|---|
| Assay | EC₅₀ | EC₅₀ | Synergy | Log | Antagonism | Log | |
| Endpoint | (nM)# | (μg/mL)# | Volume* | Volume* | Volume* | Volume* | Conclusion |
| HBV DNA | 0.015 | 0.184 | 0, 5.2 | 0, 1.8 | −6.25, −4.80 | −1.42, −1.09 | Additive |
| HBsAg | >0.07 | 0.029 | 45.62, 148.73 | 10.39, 33.86 | 0, 0 | 0, 0 | Minor to Strong Synergy |

*at 99.9% confidence interval
#determined in an earlier separate experiment

### Example 17: In Vitro Combination of Compound 1 and TDF

### Study Goal:

To determine whether a two-drug combination of Compound 1 and tenofovir disoproxil fumarate (TDF) is additive, synergistic or antagonistic in vitro, using HBV-infected human primary hepatocytes in a cell culture model system.

### Results and Conclusion:

TDF (concentration range of 10 nM to 0.123 nM in a 3-fold dilution series and 5 point titration) was tested in combination with Compound 1 (concentration range of 1.0 ng/mL to 0.012 ng/mL in a 3-fold dilution series and 5 point titration), on three replicate plates in each of two separate experimental trials The average % inhibition in HBV DNA and HBsAg, and standard deviations of 3 replicates observed either with TDF or Compound 1 treatments alone or in combination are shown in Tables 3A, 3B, 3C, and 3D as indicated below. The EC₅₀ values of TDF and Compound 1 were determined in an earlier experiment and are shown in Table 3E.

When the observed values of a two-inhibitor combination were compared to what is expected from additive interaction for the above concentration range, the combination effects ranged from additive for HBV DNA inhibition, with no significant synergy or antagonism, to synergistic for HBsAg inhibition, as per MacSynergy II analysis, and using the interpretive criteria described by Prichard and Shipman (1992) (Table 3). No significant inhibition of cell viability was observed by microscopy or CellTiter-Glo assay.

TABLE 3A
Effect on HBV DNA in In Vitro Combination of Compound 1 and TDF

| [DRUG] | | | | | | | AVERAGE % INHIBITION |
|---|---|---|---|---|---|---|---|
| TDF | | | | | | | Horizontal axis: |
| nM | 0.00 | 0.01 | 0.037 | 0.11 | 0.33 | 1.00 | Compound 1 (μg/mL) |
| 10.0 | 92.45 | 92.18 | 93.08 | 93.36 | 93.28 | 92.41 | |
| 3.33 | 88.11 | 89.33 | 91.5 | 92.28 | 91.87 | 92 | |
| 1.11 | 73.01 | 79.7 | 84.56 | 87.17 | 89.24 | 89.83 | |
| 0.37 | 44.62 | 59.13 | 69.72 | 74.68 | 81.8 | 85.07 | |
| 0.12 | 36.1 | 47.29 | 61.09 | 68.19 | 76.24 | 79.13 | |
| 0.00 | 0 | 38.08 | 49.14 | 69.25 | 71.92 | 75.74 | |

| [DRUG] | | | | | | | STANDARD DEVIATION (%) |
|---|---|---|---|---|---|---|---|
| TDF | | | | | | | Horizontal axis: |
| nM | 0.00 | 0.01 | 0.037 | 0.11 | 0.33 | 1.00 | Compound 1 (μg/mL) |
| 10.0 | 1.82 | 0.79 | 0.62 | 0.28 | 0.43 | 0.44 | |
| 3.33 | 1.37 | 2.59 | 0.95 | 1.15 | 0.48 | 0.87 | |
| 1.11 | 2.74 | 2.48 | 2.97 | 1.21 | 2.42 | 2.34 | |
| 0.37 | 6.17 | 8.21 | 1.83 | 1.1 | 2.72 | 2.17 | |
| 0.12 | 5.09 | 9.11 | 2.74 | 8.01 | 3.06 | 2.49 | |
| 0.00 | 12.18 | 6.65 | 6.83 | 9.78 | 2.03 | 2.75 | |

| [DRUG] | | | | | | | ADDITIVE % INHIBITION |
|---|---|---|---|---|---|---|---|
| TDF | | | | | | | Horizontal axis: |
| nM | 0.00 | 0.01 | 0.037 | 0.11 | 0.33 | 1.00 | Compound 1 (μg/mL) |
| 10.0 | 92.45 | 95.33 | 96.16 | 97.68 | 97.88 | 98.17 | |
| 3.33 | 88.11 | 92.64 | 93.95 | 96.34 | 96.66 | 97.12 | |
| 1.11 | 73.01 | 83.29 | 86.27 | 91.7 | 92.42 | 93.45 | |
| 0.37 | 44.62 | 65.71 | 71.83 | 82.97 | 84.45 | 86.56 | |
| 0.12 | 36.1 | 60.43 | 67.5 | 80.35 | 82.06 | 84.5 | |
| 0.00 | 0 | 38.08 | 49.14 | 69.25 | 71.92 | 75.74 | |

| [DRUG] | | | | | | | SYNERGY PLOT (99.9%) |
|---|---|---|---|---|---|---|---|
| TDF | | | | | | | Bonferroni Adj. |
| nM | 0.00 | 0.01 | 0.037 | 0.11 | 0.33 | 1.00 | 98% |
| 10.0 | 0 | −0.55011 | −1.03958 | −3.39852 | −3.18487 | −4.31 | SYNERGY 0 |
| 3.33 | 0 | 0 | 0 | −0.27535 | −3.21032 | −2.25683 | log volume 0 |
| 1.11 | 0 | 0 | 0 | −0.54789 | 0 | 0 | |
| 0.37 | 0 | 0 | 0 | −4.6699 | 0 | 0 | ANTAGONISM −23.45 |
| 0.12 | 0 | 0 | 0 | 0 | 0 | 0 | log volume −5.34 |
| 0.00 | 0 | 0 | 0 | 0 | 0 | 0 | |

TABLE 3B
Effect on HBV DNA in In Vitro Combination of Compound 1 and TDF

| [DRUG] | | | | | | | AVERAGE % INHIBITION |
|---|---|---|---|---|---|---|---|
| TDF | | | | | | | Horizontal axis: |
| nM | 0.00 | 0.01 | 0.037 | 0.11 | 0.33 | 1.00 | Compound 1 (μg/mL) |
| 10.0 | 92.76 | 93.1 | 93.61 | 93.48 | 94.1 | 92.64 | |
| 3.33 | 88.46 | 91.77 | 93.27 | 93.04 | 93.31 | 92.62 | |
| 1.11 | 75.41 | 80.82 | 85.41 | 88.42 | 89.66 | 90.47 | |
| 0.37 | 44.28 | 67.4 | 72.43 | 78.24 | 84.78 | 86.31 | |
| 0.12 | 22.22 | 51.59 | 59.77 | 69.56 | 79.39 | 82.08 | |
| 0.00 | 0 | 36.55 | 50.18 | 57.43 | 72.57 | 76.4 | |

| [DRUG] | | | | | | | STANDARD DEVIATION (%) |
|---|---|---|---|---|---|---|---|
| TDF | | | | | | | Horizontal axis: |
| nM | 0.00 | 0.01 | 0.037 | 0.11 | 0.33 | 1.00 | Compound 1 (μg/mL) |
| 10.0 | 1.87 | 1.23 | 1.45 | 1.59 | 0.73 | 0.85 | |
| 3.33 | 1.21 | 1.41 | 0.72 | 1.33 | 1.53 | 0.74 | |
| 1.11 | 3.48 | 4.59 | 0.95 | 0.9 | 1.99 | 0.65 | |
| 0.37 | 2.67 | 4.11 | 2.6 | 0.76 | 0.32 | 0.15 | |
| 0.12 | 18.63 | 5.95 | 10.09 | 1.99 | 1.68 | 1.43 | |
| 0.00 | 22.08 | 6.33 | 9.48 | 5.68 | 2.08 | 4.14 | |

| [DRUG] | | | | | | | ADDITIVE % INHIBITION |
|---|---|---|---|---|---|---|---|
| TDF | | | | | | | Horizontal axis: |
| nM | 0.00 | 0.01 | 0.037 | 0.11 | 0.33 | 1.00 | Compound 1 (μg/mL) |
| 10.0 | 92.76 | 95.41 | 96.39 | 96.92 | 98.01 | 98.29 | |
| 3.33 | 88.46 | 92.68 | 94.25 | 95.09 | 96.83 | 97.28 | |
| 1.11 | 75.41 | 84.4 | 87.75 | 89.53 | 93.25 | 94.2 | |
| 0.37 | 44.28 | 64.65 | 72.24 | 76.28 | 84.72 | 86.85 | |
| 0.12 | 22.22 | 50.65 | 61.25 | 66.89 | 78.66 | 81.64 | |
| 0.00 | 0 | 36.55 | 50.18 | 57.43 | 72.57 | 76.4 | |

| [DRUG] | | | | | | | SYNERGY PLOT (99.9%) |
|---|---|---|---|---|---|---|---|
| TDF | | | | | | | Bonferroni Adj. |
| nM | 0.00 | 0.01 | 0.037 | 0.11 | 0.33 | 1.00 | 98% |
| 10.0 | 0 | 0 | 0 | 0 | −1.50757 | −2.85265 | SYNERGY 0 |
| 3.33 | 0 | 0 | 0 | 0 | 0 | −2.22466 | log volume 0 |
| 1.11 | 0 | 0 | 0 | 0 | 0 | −1.59085 | ANTAGONISM −8.22 |
| 0.37 | 0 | 0 | 0 | 0 | 0 | −0.04635 | log volume −1.87 |
| 0.12 | 0 | 0 | 0 | 0 | 0 | 0 | |
| 0.00 | 0 | 0 | 0 | 0 | 0 | 0 | |

TABLE 3C
Effect on HBsAg in In Vitro Combination of Compound 1 and TDF

| [DRUG] | | | | | | | AVERAGE % INHIBITION |
|---|---|---|---|---|---|---|---|
| TDF | | | | | | | Horizontal axis: |
| nM | 0.00 | 0.01 | 0.037 | 0.11 | 0.33 | 1.00 | Compound 1 (μg/mL) |
| 10.0 | 18.7 | 56.34 | 68.58 | 79.56 | 86.39 | 91.14 | |
| 3.33 | 9.7 | 52.65 | 66.31 | 79.91 | 85.85 | 90.92 | |
| 1.11 | 2.8 | 50.97 | 66.56 | 79.74 | 85.63 | 90.39 | |
| 0.37 | −1.3 | 47.03 | 66.16 | 78.79 | 85.74 | 90.24 | |
| 0.12 | −1.47 | 49.98 | 64.28 | 77.48 | 84.76 | 88.7 | |
| 0.00 | 0 | 41.17 | 56.07 | 71.38 | 79.87 | 86.97 | |

| [DRUG] | | | | | | | STANDARD DEVIATION (%) |
|---|---|---|---|---|---|---|---|
| TDF | | | | | | | Horizontal axis: |
| nM | 0.00 | 0.01 | 0.037 | 0.11 | 0.33 | 1.00 | Compound 1 (μg/mL) |
| 10.0 | 5.95 | 1.83 | 1.76 | 0.63 | 0.86 | 1.38 | |
| 3.33 | 9.12 | 0.38 | 1.12 | 1.45 | 1.12 | 1.06 | |
| 1.11 | 4.81 | 1.89 | 2.12 | 1.02 | 0.32 | 0.37 | |
| 0.37 | 13.93 | 9.12 | 1.84 | 1.4 | 0.93 | 0.28 | |
| 0.12 | 5.26 | 2.29 | 3.17 | 1.34 | 0.27 | 0.86 | |
| 0.00 | 6.4 | 2.75 | 1.66 | 0.73 | 0.84 | 1.04 | |

| [DRUG] | | | | | | | ADDITIVE % INHIBITION |
|---|---|---|---|---|---|---|---|
| TDF | | | | | | | Horizontal axis: |
| nM | 0.00 | 0.01 | 0.037 | 0.11 | 0.33 | 1.00 | Compound 1 (μg/mL) |
| 10.0 | 18.7 | 52.17 | 64.28 | 76.73 | 83.63 | 89.41 | |
| 3.33 | 9.7 | 46.88 | 60.33 | 74.16 | 81.82 | 88.23 | |
| 1.11 | 2.8 | 42.82 | 57.3 | 72.18 | 80.43 | 87.33 | |
| 0.37 | −1.3 | 40.41 | 55.5 | 71.01 | 79.61 | 86.8 | |
| 0.12 | −1.47 | 40.31 | 55.42 | 70.96 | 79.57 | 86.78 | |
| 0.00 | 0 | 41.17 | 56.07 | 71.38 | 79.87 | 86.97 | |

| [DRUG] | | | | | | | SYNERGY PLOT (99.9%) |
|---|---|---|---|---|---|---|---|
| TDF | | | | | | | Bonferroni Adj. |
| nM | 0.00 | 0.01 | 0.037 | 0.11 | 0.33 | 1.00 | 98% |
| 10.0 | 0 | 0 | 0 | 0.75667 | 0 | 0 | SYNERGY 45.21 |
| 3.33 | 0 | 4.51942 | 2.29408 | 0.97805 | 0.34408 | 0 | log volume 10.29 |
| 1.11 | 0 | 1.93001 | 2.28308 | 4.20318 | 4.14688 | 1.84233 | ANTAGONISM 0 |
| 0.37 | 0 | 0 | 4.60456 | 3.1726 | 3.06937 | 2.51852 | log volume 0 |
| 0.12 | 0 | 2.13361 | 0 | 2.11006 | 4.30143 | 0 | |
| 0.00 | 0 | 0 | 0 | 0 | 0 | 0 | |

TABLE 3D
Effect on HBsAg in In Vitro Combination of Compound 1 and TDF

| [DRUG] | | | | | | | AVERAGE % INHIBITION |
|---|---|---|---|---|---|---|---|
| TDF | | | | | | | Horizontal axis: |
| nM | 0.00 | 0.01 | 0.037 | 0.11 | 0.33 | 1.00 | Compound 1 (μg/mL) |
| 10.0 | 5.83 | 55.51 | 69.87 | 82.09 | 89.03 | 93.1 | |
| 3.33 | 4.83 | 56.16 | 72.21 | 82.01 | 89.1 | 93.38 | |
| 1.11 | −6.79 | 56.21 | 71.29 | 82.54 | 89.23 | 92.78 | |
| 0.37 | −9.3 | 54.7 | 70.6 | 82.39 | 88.17 | 92.53 | |
| 0.12 | −10.84 | 53.5 | 69.22 | 81.96 | 87.91 | 92.13 | |
| 0.00 | 0 | 43.06 | 60.86 | 75.5 | 83.9 | 89.48 | |

| [DRUG] | | | | | | | STANDARD DEVIATION (%) |
|---|---|---|---|---|---|---|---|
| TDF | | | | | | | Horizontal axis: |
| nM | 0.00 | 0.01 | 0.037 | 0.11 | 0.33 | 1.00 | Compound 1 (μg/mL) |
| 10.0 | 6.2 | 3.38 | 2.15 | 1.05 | 0.2 | 0.69 | |
| 3.33 | 2.51 | 3.53 | 1.14 | 0.82 | 1.11 | 0.57 | |
| 1.11 | 4.29 | 1.83 | 0.82 | 0.77 | 0.85 | 1.01 | |
| 0.37 | 7.88 | 1.22 | 3.16 | 1.1 | 0.49 | 0.55 | |
| 0.12 | 1.33 | 1.03 | 1.59 | 1.71 | 1.15 | 0.56 | |
| 0.00 | 4.21 | 3.91 | 2.62 | 2.66 | 0.76 | 0.64 | |

| [DRUG] | | | | | | | ADDITIVE % INHIBITION |
|---|---|---|---|---|---|---|---|
| TDF | | | | | | | Horizontal axis: |
| nM | 0.00 | 0.01 | 0.037 | 0.11 | 0.33 | 1.00 | Compound 1 (μg/mL) |
| 10.0 | 5.83 | 46.38 | 63.14 | 76.93 | 84.84 | 90.09 | |
| 3.33 | 4.83 | 45.81 | 62.75 | 76.68 | 84.68 | 89.99 | |
| 1.11 | −6.79 | 39.19 | 58.2 | 73.84 | 82.81 | 88.77 | |
| 0.37 | −9.3 | 37.76 | 57.22 | 73.22 | 82.4 | 88.5 | |
| 0.12 | −10.84 | 36.89 | 56.62 | 72.84 | 82.15 | 88.34 | |
| 0.00 | 0 | 43.06 | 60.86 | 75.5 | 83.9 | 89.48 | |

| [DRUG] | | | | | | | SYNERGY PLOT (99.9%) |
|---|---|---|---|---|---|---|---|
| TDF | | | | | | | Bonferroni Adj. |
| nM | 0.00 | 0.01 | 0.037 | 0.11 | 0.33 | 1.00 | 98% |
| 10.0 | 0 | 0 | 0 | 1.70445 | 3.5318 | 0.73921 | SYNERGY 104.29 |
| 3.33 | 0 | 0 | 5.70826 | 2.63138 | 0.76699 | 1.51413 | log volume 23.74 |
| 1.11 | 0 | 10.99747 | 10.39138 | 6.16593 | 3.62265 | 0.68609 | |
| 0.37 | 0 | 12.92498 | 2.98044 | 5.5499 | 4.15741 | 2.21995 | ANTAGONISM 0 |
| 0.12 | 0 | 13.22027 | 7.36731 | 3.49239 | 1.97535 | 1.94704 | log volume 0 |
| 0.00 | 0 | 0 | 0 | 0 | 0 | 0 | |

TABLE 3E
Summary of results of in vitro combination studies of Compound 1 and TDF in PHH cell culture system

| HBV | TDF | Cmpd 1 | | Synergy | | Antagonism | |
|---|---|---|---|---|---|---|---|
| Assay | EC₅₀ | EC₅₀ | Synergy | Log | Antagonism | Log | |
| Endpoint | (nM)# | (μg/mL)# | Volume* | Volume* | Volume* | Volume* | Conclusion |
| HBV DNA | 0.42 | 0.184 | 0, 0 | 0, 0 | −23.45, −8.22 | −5.34, −1.87 | Additive |
| HBsAg | >10 | 0.029 | 45.21, 104.29 | 10.29, 23.74 | 0, 0 | 0, 0 | Minor to Strong Synergy |

*at 99.9% confidence interval
#determined in an earlier separate experiment

### Example 18

A mouse model of hepatitis B virus (HBV) was used to assess the anti-HBV effects of a HBV-targeting GalNAc-siRNA (N-acetylgalactosamine-conjugated short interfering RNA) and a small molecule inhibitor of HBV encapsidation, in combination with each other and in combination with an approved nucleos(t)ide analog compound. The relative inhibitory activities of the three anti-HBV agents were evaluated and compared as stand-alone treatments, in all possible dual combinations, and as a triple combination.

The HBV GalNAc-siRNA has the following structure as follows. In certain embodiments, the siRNA of the siRNA conjugate is siRNA 1 below. In certain embodiments, the siRNA of the siRNA conjugate is siRNA 2 below. In the experiments described hereinbelow, the siRNA of the siRNA conjugate is siRNA 2 below. The compound of formula (1) is depicted below, wherein the siRNA of the siRNA conjugate is siRNA 2.

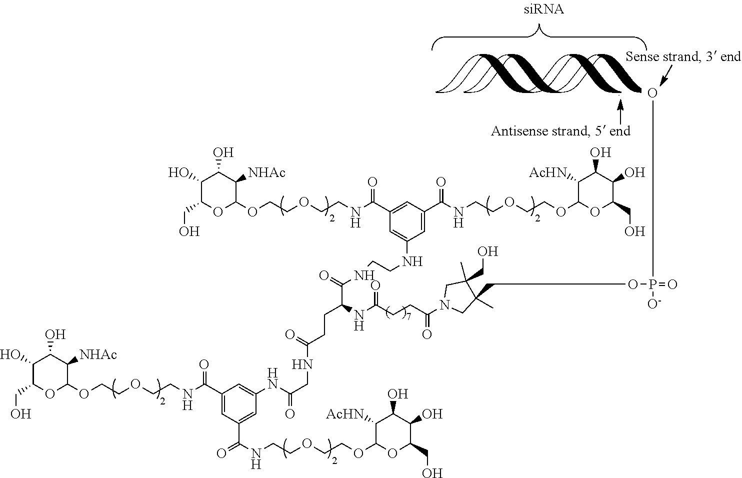

| Name | Sense Sequence (5′-3′) | Antisense Sequence (5′-3′) |
|---|---|---|
| siRNA 1 | usgscaCUUcgcuucaccu | asGsgugaagcgaagUgCacascsgU |
| siRNA 2 | gsusgcACUucgcuucaca | usGsugaagcgaaguGcAcacsgsgU |

2′-O-Methyl nucleotides = lower case
2′-Fluoro nucleotides = UPPER_CASE
Phosphorothioate linker = s
Unmodified = UPPER CASE

The inhibitor of HBV encapsidation has the following structure:

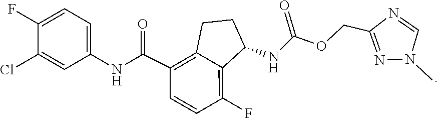 [SMILES: Cn1cnc(COC(=O)N[C@H]2CCc3c(C(=O)Nc4ccc(F)c(Cl)c4)ccc(F)c32)n1]

There are a number of nucleos(t)ide analogs approved for the treatment of chronic hepatitis B infection, and their mode of action is inhibition of HBV polymerase/reverse transcriptase. In this study, tenofovir disoproxil fumarate (TDF) was utilized as an example of this class of drug.

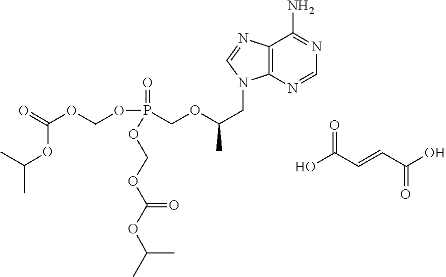 [SMILES: CC(C)OC(=O)OCOP(=O)(CO[C@H](C)Cn1cnc2c(N)ncnc21)OCOC(=O)OC(C)C ; O=C(O)/C=C/C(=O)O]

Prior to treatment start, 1×10¹¹ viral genomes of an adeno-associated virus (AAV) vector carrying a 1.3-fold overlength copy of an HBV genome (serotype Ayw, genotype D) was administered to C57BL/6 mice via intravenous injection. Introduction of this viral vector results in the expression of HBV DNA and HBV surface antigen (HBsAg) amongst other HBV products. Serum HBV DNA levels in mice was measured using a quantitative polymerase chain reaction (QPCR) assay, HBsAg in serum and liver of mice was measured using an enzyme-linked immunosorbent assay (ELISA), and anti-HBsAg antibodies were measured using ELISA. Animals were sorted (randomized) into groups based on a lack of detectable anti-HBsAg antibodies as well as serum HBV DNA and HBsAg levels such that a) all animals were confirmed to express both markers and b) mean serum HBV DNA and mean serum HBsAg values were similar between groups 4-7 days before starting treatments.

Animals were treated with HBV-targeting siRNA as follows: On each of Days 0 and 28, 3 mg/kg siRNA was administered subcutaneously for a total of two doses across the duration of the study. Animals were treated with vehicle-only control, HBV encapsidation inhibitor and/or TDF as follows: Starting on Day 0 and ending on Day 41, daily doses of 100 mg/kg encapsidation inhibitor, and/or 1 mg/kg TDF were administered orally for a total of 42 doses across the duration of the study.

Treatment effects on serum HBV DNA were determined by collecting a small amount of blood on Days 0 (pre-treatment) and 14, as well as from terminal blood collections at Day 42. Treatment effects on HBsAg in serum and liver were determined from terminal sample collections at Day 42.

Table 1 shows the group mean (n=6; ±standard error of the mean) serum HBV DNA concentration expressed as log₁₀ copies/microliter. Table 2 shows the group mean (n=6; ±standard error of the mean) serum HBsAg concentration expressed as log₁₀ IU/mL and liver HBsAg concentration expressed as log₁₀ IU/mg liver protein. Any individual animal samples measured to fall below assay lower limit of quantitation (LLOQ) were reported as the LLOQ value.

The data demonstrate that anti-HBV effects were greater when agents of different drug mechanisms of action (siRNA, encapsidation inhibitor, nucleos(t)ide analog) were administered concurrently. The combination of the three agents together resulted in greater HBV DNA inhibition (−2.23 log₁₀ decrease from Day 0 to Day 42) than any single treatment alone (maximum 0.73 log₁₀ decrease, for TDF) or any combination of two agents (maximum 1.92 log₁₀ decrease, for siRNA plus TDF). HBsAg inhibition occurred in all treatment regimens that included the HBV siRNA agent, and while combination with the other two agents did not appreciably change the anti-HBsAg effect in serum, the triple combination regimen did cause the largest decrease in liver HBsAg (−1.78 log₁₀ reduction versus Control Group 1, as opposed to −1.36 log₁₀ reduction for siRNA alone).

TABLE 1
Serum HBV DNA in a mouse model of HBV infection following every-
4-weeks subcutaneous administration of an HBV-targeting GalNAc-
siRNA, once-daily oral administration of an HBV encapsidation
inhibitor and/or once-daily oral administration of the nucleos(t)ide
analog TDF separately and in dual and triple combination.

| | Subcutaneously | Orally Dosed | Serum HBV DNA | | |
|---|---|---|---|---|---|
| | Dosed Agent 1 | Agent 2 and/or 3 | Day 0 | Day 14 | Day 42 |
| Group 1 | none | Vehicle Only | 4.40 ± 0.08 | 4.67 ± 0.06 | 5.02 ± 0.09 |
| Group 2 | none | TDF | 4.31 ± 0.10 | 3.11 ± 0.15 | 3.58 ± 0.14 |
| Group 3 | none | Encapsidation | 4.45 ± 0.13 | 3.89 ± 0.07 | 4.49 ± 0.07 |
| | | Inhibitor | | | |
| Group 4 | GalNAc- | none | 4.28 ± 0.18 | 2.94 ± 0.25 | 3.58 ± 0.20 |
| | siRNA | | | | |
| Group 5 | none | Encapsidation | 4.17 ± 0.16 | 2.49 ± 0.09 | 2.99 ± 0.08 |
| | | Inhibitor, and | | | |
| | | TDF | | | |
| Group 6 | GalNAc- | TDF | 4.28 ± 0.06 | 2.08 ± 0.00 | 2.36 ± 0.06 |
| | siRNA | | | | |
| Group 7 | GalNAc- | Encapsidation | 4.49 ± 0.09 | 3.26 ± 0.12 | 3.31 ± 0.21 |
| | siRNA | Inhibitor | | | |
| Group 8 | GalNAc- | Encapsidation | 4.50 ± 0.10 | 2.10 ± 0.02 | 2.27 ± 0.06 |
| | siRNA | Inhibitor, and | | | |
| | | TDF | | | |

TABLE 2
Serum and liver HBsAg in a mouse model of HBV infection
following every-4-weeks subcutaneous administration
of an HBV-targeting GalNAc-siRNA, once-daily oral administration
of an HBV encapsidation inhibitor and/or once-daily
oral administration of the nucleos(t)ide analog TDF
separately and in dual and triple combination.

| | | Orally Dosed | Study Day 42 | |
|---|---|---|---|---|
| | Subcutaneously | Agent 2 and/ | Serum | Liver |
| | Dosed Agent 1 | or 3 | HBsAg | HBsAg |
| Group 1 | none | Vehicle Only | 4.59 ± 0.04 | 1.76 ± 0.06 |
| Group 2 | none | TDF | 4.70 ± 0.04 | 1.77 ± 0.03 |
| Group 3 | none | Encapsidation | 4.88 ± 0.02 | 2.03 ± 0.03 |
| | | Inhibitor | | |
| Group 4 | GalNAc- | none | 2.76 ± 0.22 | 0.40 ± 0.13 |
| | siRNA | | | |
| Group 5 | none | Encapsidation | 4.80 ± 0.02 | 1.83 ± 0.07 |
| | | Inhibitor, | | |
| | | and TDF | | |
| Group 6 | GalNAc- | TDF | 3.13 ± 0.19 | 0.60 ± 0.06 |
| | siRNA | | | |
| Group 7 | GalNAc- | Encapsidation | 3.06 ± 0.36 | 0.97 ± 0.10 |
| | siRNA | Inhibitor | | |
| Group 8 | GalNAc- | Encapsidation | 3.25 ± 0.14 | −0.02 ± 0.03 |
| | siRNA | Inhibitor, | | |
| | | and TDF | | |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method of ameliorating at least one symptom of HBV infection in a human subject infected with HBV, the method comprising the steps of:
(a) administering to the human subject a GalNAc-siRNA conjugate, wherein the siRNA portion of the conjugate targets a portion of the HBV genome; and
(b) administering to the subject at least one anti-HBV agent selected from the group consisting of: an RNA destabilizer; a capsid inhibitor; a reverse transcriptase inhibitor; an immunostimulator; a cccDNA formation inhibitor; and an oligomeric nucleotide targeted to the Hepatitis B genome.

2. The method of claim 1, wherein the method comprises administering to the subject an RNA destabilizer.

3. The method of any one of claims 1-2, wherein the method comprises administering to the subject a capsid inhibitor.

4. The method of any one of claims 1-3, wherein the method comprises administering to the subject a reverse transcriptase inhibitor.

5. The method of any one of claims 1-4, wherein the method comprises administering to the subject an immunostimulator.

6. The method of any one of claims 1-5, wherein the method comprises administering to the subject a cccDNA formation inhibitor.

7. The method of any one of claims 1-6, wherein the method comprises administering to the subject an oligomeric nucleotide targeted to the Hepatitis B genome.

8. The method of any one of claims 1-7, wherein the GalNAc-siRNA conjugate is administered subcutaneously.

9. The method of any one of claims 1-8, wherein the anti-HBV agent of step (b) is administered orally.

10. The method of any one of claims 1-9, wherein the anti-HBV agent of step (b) is administered orally in pill form.

11. The method of any one of claims 1-10, wherein the reverse transcriptase inhibitor is a nucleoside analogue HBV reverse transcriptase inhibitor.

12. The method of any one of claims 1-11, wherein the GalNAc-siRNA conjugate is a compound of formula (V), or a salt thereof, as described in Examples 1-4.

13. The method of any one of claims 1-12, wherein the RNA destabilizer is a compound of formula (VI), or a salt thereof, as described in Examples 1-4.

14. The method of any one of claims 1-13, wherein the capsid inhibitor is a compound of formula (VII), or a salt thereof, as described in Examples 1-4.

15. The method of any one of claims 1-14, wherein the immunostimulator is a pegylated interferon (PEG-IFN).

16. The method of any one of claims 1-15, wherein the immunostimulator is pegylated interferon alpha 2a (PEG-IFNα2a).

17. The method of any one of claims 1-16, wherein the reverse transcriptase inhibitor is tenofovir alafenamide fumarate (TAF).

18. The method of any one of claims 1-16, wherein the reverse transcriptase inhibitor is tenofovir disoproxil fumarate (TDF).

19. The method of any one of claims 1-16, wherein the reverse transcriptase inhibitor is entecavir (ETV).

20. The method of any one of claims 1-16, comprising the administration of entecavir and tenofovir disoproxil fumarate

21. The method of any one of claims 1-20, wherein the GalNAc-siRNA conjugate is administered simultaneously with the anti-HBV agent of step (b).

22. The method of any one of claims 1-20, wherein the GalNAc-siRNA conjugate and the anti-HBV agent of step (b) are administered sequentially.

23. The method of any one of claims 1-20, wherein the GalNAc-siRNA conjugate is administered prior to the administration of the anti-HBV agent of step (b).

24. The method of any one of claims 1-20, wherein the GalNAc-siRNA conjugate is administered after the administration of the anti-HBV agent of step (b).

25. The method of any one of claims 1-24, further comprising administering at least one additional therapeutic agent to the subject.

26. A method of ameliorating at least one symptom of HDV infection in a human subject infected with HDV, the method comprising the steps of:
(a) administering to the human subject a GalNAc-siRNA conjugate, wherein the siRNA portion of the conjugate targets a portion of the HBV genome; and
(b) administering to the subject at least one anti-HBV agent selected from the group consisting of: an RNA destabilizer; a capsid inhibitor; a reverse transcriptase inhibitor; an immunostimulator; a cccDNA formation inhibitor; and an oligomeric nucleotide targeted to the Hepatitis B genome.

27. The use of a combination of a GalNAc-siRNA conjugate, wherein the siRNA portion of the conjugate targets a portion of the HBV genome, and at least one anti-HBV agent selected from the group consisting of: an RNA destabilizer; a capsid inhibitor; a reverse transcriptase inhibitor; an immunostimulator; a cccDNA formation inhibitor; and an oligomeric nucleotide targeted to the Hepatitis B genome, to ameliorate at least one symptom of HBV infection in a human subject.

28. The use of a combination of a GalNAc-siRNA conjugate, wherein the siRNA portion of the conjugate targets a portion of the HBV genome, and at least one anti-HBV agent selected from the group consisting of: an RNA destabilizer; a capsid inhibitor; a reverse transcriptase inhibitor; an immunostimulator; a cccDNA formation inhibitor; and an oligomeric nucleotide targeted to the Hepatitis B genome, to treat HBV infection in a human subject.

29. The use of a combination of a GalNAc-siRNA conjugate, wherein the siRNA portion of the conjugate targets a portion of the HBV genome, and at least one anti-HBV agent selected from the group consisting of: an RNA destabilizer; a capsid inhibitor; a reverse transcriptase inhibitor; an immunostimulator; a cccDNA formation inhibitor; and an oligomeric nucleotide targeted to the Hepatitis B genome, to treat HDV infection in a human subject.

30. A method for treating Hepatitis B in an animal comprising administering to the animal, at least two agents selected from the group consisting of:
a) a capsid inhibitor, wherein the capsid inhibitor is: 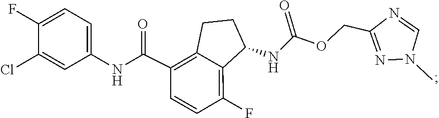 [SMILES: Cn1cnc(COC(=O)N[C@H]2CCc3c(C(=O)Nc4ccc(F)c(Cl)c4)ccc(F)c32)n1]
b) an RNA destabilizer, wherein the RNA destabilizer is: 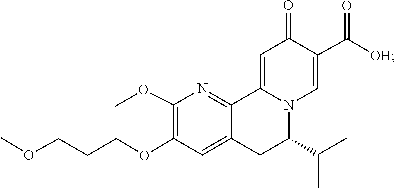 [SMILES: COCCCOc1cc2c(nc1OC)-c1cc(=O)c(C(=O)O)cn1[C@H](C(C)C)C2]
c) reverse transcriptase inhibitors selected from the group consisting of tenofovir disoproxil fumarate, tenofovir alafenamide and entecavir; and
d) oligomeric nucleotides targeted to the Hepatitis B genome.

31. The method of claim 30, wherein at least three oligomeric nucleotides targeted to the Hepatitis B genome are administered to the animal.

32. The method of claim 31, wherein oligomeric nucleotides 3m, 6m and 12m are administered to the animal.

33. The method of any one of claims 30-32, wherein at least one agent is administered orally.

34. The method of any one of claims 30-33, wherein at least one oligomeric nucleotide is administered intravenously.

35. The method of claim 30, wherein one of the following combinations of two agents is administered to the animal:
the RNA destabilizer and the capsid inhibitor;
at least one oligomeric nucleotide targeted to the Hepatitis B genome and the capsid inhibitor;
at least one oligomeric nucleotide targeted to the Hepatitis B genome and the RNA destabilizer;
at least one oligomeric nucleotide targeted to the Hepatitis B genome and a reverse transcriptase inhibitor;
the capsid inhibitor and a reverse transcriptase inhibitor; or
the RNA destabilizer and a reverse transcriptase inhibitor.

36. The method of claim 30, wherein one of the following combinations of two agents is administered to the animal:
the RNA destabilizer and the capsid inhibitor;
a combination comprising three oligomeric nucleotides targeted to the Hepatitis B genome, wherein the oligomeric nucleotides are 3m, 6m and 12m; and the capsid inhibitor;
the capsid inhibitor and tenofovir disoproxil fumarate;
the capsid inhibitor and tenofovir alafenamide;
the capsid inhibitor and entecavir;
the RNA destabilizer and tenofovir disoproxil fumarate;
the RNA destabilizer and tenofovir alafenamide; or
the RNA destabilizer and entecavir.

37. The method of claim 30, wherein one of the following combinations of three agents is administered to the animal:
the capsid inhibitor, the RNA destabilizer and a reverse transcriptase inhibitor;
the capsid inhibitor, at least one oligomeric nucleotide targeted to the Hepatitis B genome and a reverse transcriptase inhibitor;
the capsid inhibitor, the RNA destabilizer and at least one oligomeric nucleotide targeted to the Hepatitis B genome; or
the RNA destabilizer, at least one oligomeric nucleotide targeted to the Hepatitis B genome and a reverse transcriptase inhibitor.

38. The method of claim 30, wherein one of the following combinations of three agents is administered to the animal:
the capsid inhibitor, the RNA destabilizer and tenofovir disoproxil fumarate;
the capsid inhibitor, the RNA destabilizer and tenofovir alafenamide; or
the capsid inhibitor, the RNA destabilizer and entecavir.

39. A kit comprising at least two agents selected from the group consisting of:
a) a capsid inhibitor, wherein the capsid inhibitor is: 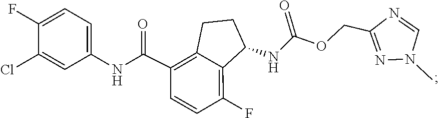 [SMILES: Cn1cnc(COC(=O)N[C@H]2CCc3c(C(=O)Nc4ccc(F)c(Cl)c4)ccc(F)c32)n1]
b) an RNA destabilizer, wherein the RNA destabilizer is: 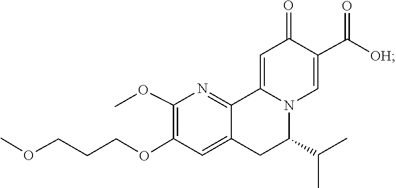 [SMILES: COCCCOc1cc2c(nc1OC)-c1cc(=O)c(C(=O)O)cn1[C@H](C(C)C)C2]
c) reverse transcriptase inhibitors selected from the group consisting of tenofovir disoproxil fumarate, tenofovir alafenamide and entecavir; and
d) oligomeric nucleotides targeted to the Hepatitis B genome;
for use in combination to treat or prevent Hepatitis B.

40. The kit of claim 39 that comprises at least three oligomeric nucleotides targeted to the Hepatitis B genome.

41. The kit of claim 40 that comprises oligomeric nucleotides 3m, 6m and 12m.

42. The kit of claim 39 that comprises one of the following combinations of two agents:
the RNA destabilizer and the capsid inhibitor;
at least one oligomeric nucleotide targeted to the Hepatitis B genome and the capsid inhibitor;
at least one oligomeric nucleotide targeted to the Hepatitis B genome and the RNA destabilizer;
at least one oligomeric nucleotide targeted to the Hepatitis B genome and a reverse transcriptase inhibitor;
the capsid inhibitor and a reverse transcriptase inhibitor; or
the RNA destabilizer and a reverse transcriptase inhibitor.

43. The kit of claim 39 that comprises one of the following combinations of two agents:
the RNA destabilizer and the capsid inhibitor;
a combination comprising three oligomeric nucleotides targeted to the Hepatitis B genome, wherein the oligomeric nucleotides are 3m, 6m and 12m; and the capsid inhibitor;
the capsid inhibitor and tenofovir disoproxil fumarate;
the capsid inhibitor and tenofovir alafenamide;
the capsid inhibitor and entecavir;
the RNA destabilizer and tenofovir disoproxil fumarate;
the RNA destabilizer and tenofovir alafenamide; or
the RNA destabilizer and entecavir.

44. The kit of claim 39 that comprises one of the following combinations of three agents:
the capsid inhibitor, the RNA destabilizer and a reverse transcriptase inhibitor;
the capsid inhibitor, at least one oligomeric nucleotide targeted to the Hepatitis B genome and a reverse transcriptase inhibitor;
the capsid inhibitor, the RNA destabilizer and at least one oligomeric nucleotide targeted to the Hepatitis B genome; or
the RNA destabilizer, at least one oligomeric nucleotide targeted to the Hepatitis B genome and a reverse transcriptase inhibitor.

45. The kit of claim 39 that comprises one of the following combinations of three agents:
the capsid inhibitor, the RNA destabilizer and tenofovir disoproxil fumarate;
the capsid inhibitor, the RNA destabilizer and tenofovir alafenamide; or
the capsid inhibitor, the RNA destabilizer and entecavir.

46. A pharmaceutical composition that comprises a pharmaceutically acceptable carrier and at least two agents selected from the group consisting of:
a) a capsid inhibitor, wherein the capsid inhibitor is: 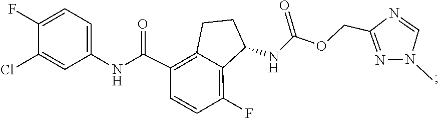 [SMILES: Cn1cnc(COC(=O)N[C@H]2CCc3c(C(=O)Nc4ccc(F)c(Cl)c4)ccc(F)c32)n1]
b) an RNA destabilizer, wherein the RNA destabilizer is: 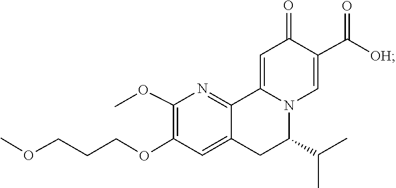 [SMILES: COCCCOc1cc2c(nc1OC)-c1cc(=O)c(C(=O)O)cn1[C@H](C(C)C)C2]
c) reverse transcriptase inhibitors selected from the group consisting of tenofovir disoproxil fumarate, tenofovir alafenamide and entecavir; and
d) oligomeric nucleotides targeted to the Hepatitis B genome.

47. The pharmaceutical composition of claim 46 that comprises at least three oligomeric nucleotides targeted to the Hepatitis B genome.

48. The pharmaceutical composition of claim 47 that comprises oligomeric nucleotides 3m, 6m and 12m.

49. The pharmaceutical composition of claim 46 that comprises one of the following combinations of two agents:
the RNA destabilizer and the capsid inhibitor;
at least one oligomeric nucleotide targeted to the Hepatitis B genome and the capsid inhibitor;
at least one oligomeric nucleotide targeted to the Hepatitis B genome and the RNA destabilizer;
at least one oligomeric nucleotide targeted to the Hepatitis B genome and a reverse transcriptase inhibitor;
the capsid inhibitor and a reverse transcriptase inhibitor; or
the RNA destabilizer and a reverse transcriptase inhibitor.

50. The pharmaceutical composition of claim 46 that comprises one of the following combinations of two agents:
the RNA destabilizer and the capsid inhibitor;
a combination comprising three oligomeric nucleotides targeted to the Hepatitis B genome, wherein the oligomeric nucleotides are 3m, 6m and 12m; and the capsid inhibitor;
the capsid inhibitor and tenofovir disoproxil fumarate;
the capsid inhibitor and tenofovir alafenamide;
the capsid inhibitor and entecavir;
the RNA destabilizer and tenofovir disoproxil fumarate;
the RNA destabilizer and tenofovir alafenamide; or
the RNA destabilizer and entecavir.

51. The pharmaceutical composition of claim 46 that comprises one of the following combinations of three agents:
the capsid inhibitor, the RNA destabilizer and a reverse transcriptase inhibitor;
the capsid inhibitor, at least one oligomeric nucleotide targeted to the Hepatitis B genome and a reverse transcriptase inhibitor;
the capsid inhibitor, the RNA destabilizer and at least one oligomeric nucleotide targeted to the Hepatitis B genome; or
the RNA destabilizer, at least one oligomeric nucleotide targeted to the Hepatitis B genome and a reverse transcriptase inhibitor.

52. The pharmaceutical composition of claim 46 that comprises one of the following combinations of three agents:
the capsid inhibitor, the RNA destabilizer and tenofovir disoproxil fumarate;
the capsid inhibitor, the RNA destabilizer and tenofovir alafenamide; or
the capsid inhibitor, the RNA destabilizer and entecavir.

53. A combination of at least two agents selected from the group consisting of:
a) a capsid inhibitor, wherein the capsid inhibitor is: 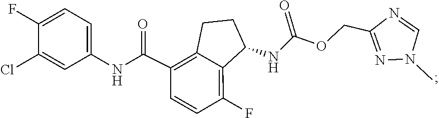 [SMILES: Cn1cnc(COC(=O)N[C@H]2CCc3c(C(=O)Nc4ccc(F)c(Cl)c4)ccc(F)c32)n1]
b) an RNA destabilizer, wherein the RNA destabilizer is: 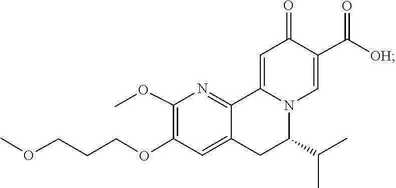 [SMILES: COCCCOc1cc2c(nc1OC)-c1cc(=O)c(C(=O)O)cn1[C@H](C(C)C)C2]
c) reverse transcriptase inhibitors selected from the group consisting of tenofovir disoproxil fumarate, tenofovir alafenamide and entecavir; and
d) oligomeric nucleotides targeted to the Hepatitis B genome,
for use in treating Hepatitis B in an animal.

54. The use of a combination of at least two agents selected from the group consisting of:
a) a capsid inhibitor, wherein the capsid inhibitor is: 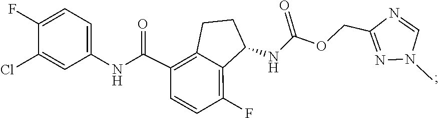 [SMILES: Cn1cnc(COC(=O)N[C@H]2CCc3c(C(=O)Nc4ccc(F)c(Cl)c4)ccc(F)c32)n1]
b) an RNA destabilizer, wherein the RNA destabilizer is: 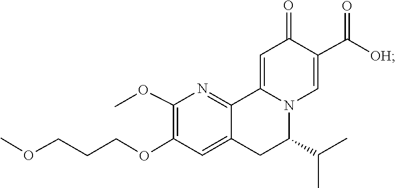 [SMILES: COCCCOc1cc2c(nc1OC)-c1cc(=O)c(C(=O)O)cn1[C@H](C(C)C)C2]
c) reverse transcriptase inhibitors selected from the group consisting of tenofovir disoproxil fumarate, tenofovir alafenamide and entecavir; and
d) oligomeric nucleotides targeted to the Hepatitis B genome,
in the manufacture of a medicament for the treatment of Hepatitis B in an animal.

55. A method for treating Hepatitis D in an animal comprising administering to the animal, at least two agents selected from the group consisting of:
a) a capsid inhibitor, wherein the capsid inhibitor is: 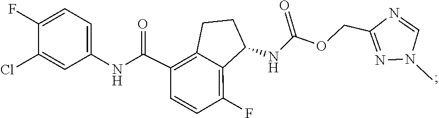 [SMILES: Cn1cnc(COC(=O)N[C@H]2CCc3c(C(=O)Nc4ccc(F)c(Cl)c4)ccc(F)c32)n1]
b) an RNA destabilizer, wherein the RNA destabilizer is: 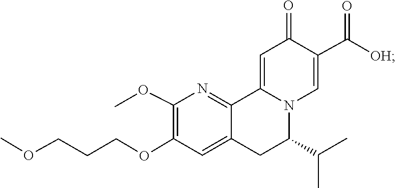 [SMILES: COCCCOc1cc2c(nc1OC)-c1cc(=O)c(C(=O)O)cn1[C@H](C(C)C)C2]
c) reverse transcriptase inhibitors selected from the group consisting of tenofovir disoproxil fumarate, tenofovir alafenamide and entecavir; and
d) oligomeric nucleotides targeted to the Hepatitis B genome.
